(19) United States
(12) Patent Application Publication
(10) Pub. No.: US 2026/0224714 A1
(43) Pub. Date: Aug. 6, 2026

(54) CYCLING LYSOSOMAL TARGETING ANTIBODY CONJUGATES

(71) Applicant: Lycia Therapeutics, Inc., South San Francisco, CA (US)

(72) Inventors: Nicholas Lind, Lafayette, CA (US); Tao Chen, Palo Alto, CA (US); Christos Kougentakis, San Francisco, CA (US); Jason G. Lewis, Castro Valley, CA (US); Matthew Shurtleff, Redwood City, CA (US); Steven Staben, Emerald Hills, CA (US); Eric D. Turtle, Belmont, CA (US); Richard Yau, San Francisco, CA (US); Shuai Zheng, Richmond, CA (US); Thomas-Toan Tran, San Jose, CA (US)

(21) Appl. No.: 19/148,839

(22) PCT Filed: Jan. 17, 2024
(86) PCT No.: PCT/US2024/011897

§ 371 Date: Jul. 17, 2025

Related U.S. Application Data
(60) Provisional application No. 63/480,510, filed on Jan. 18, 2023.

(51) Int. Cl. A61K 47/55; A61K 47/54; A61K 47/68

(52) U.S. Cl. CPC A61K 47/55; A61K 47/549; A61K 47/6873

(57) ABSTRACT

The present disclosure provides conjugates comprising a moiety that targets a lysosomal targeting molecule, a linker, and an antibody that specifically binds a cell surface target molecule or extracellular target molecule that is targeted for degradation.

### CROSS-REFERENCE TO RELATED APPLICATIONS

This application claims the benefit under 35 U.S.C. § 119(e) to U.S. Provisional Application No. 63/480,510, filed Jan. 18, 2023, which is hereby incorporated by reference in its entirety.

### BACKGROUND

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

### SUMMARY

The present disclosure provides a class of conjugate compounds that include a ligand moiety that binds to a lysosomal targeting molecule (e.g., a receptor) and an antibody or antibody fragment that binds to a cell surface, including transmembrane, target molecule or extracellular target molecule. The conjugates can facilitate degradation of a target and can repeatedly “cycle” into and out of a cell. Such cycling enables duration of activity on the order of hours to days, and the ability for a single conjugate to facilitate lysosomal degradation of multiple targets. In some embodiments, binding of the ligand moiety to the lysosomal targeting molecule can trigger internalization of the lysosomal targeting molecule and conjugate. In one embodiment, is provided a ligand moiety conjugated via a linker to a target-binding moiety wherein the ligand moiety binds a lysosomal targeting molecule extracellularly; the target-binding moiety binds a target molecule extracellularly; the target-binding moiety dissociates from the target molecular intraendosomally and the conjugate is externalized from a cell. In some embodiments, the conjugate comprises a target-binding moiety having a higher binding affinity for the target molecule at an extracellular pH than at an intraendosomal pH, while at the same time, having one or more of a) a target-binding moiety having a higher affinity for FcRn at an intraendosomal pH than at an extracellular pH; b) a ligand moiety, X, having an equal binding affinity for the lysosomal targeting molecule extracellularly and intraendosomally; c) a ligand moiety, X, having an equal binding affinity for the lysosomal targeting molecule at an extracellular pH and at an intraendosomal pH; and/or d) a ligand moiety, X, having an equal binding affinity for the lysosomal targeting molecule at an extracellular Ca²⁺ concentration and at an intraendosomal Ca²⁺ concentration. In some embodiments, the conjugate exhibits duration of activity on the order of hours to days.

In some embodiments, the conjugate is configured to exhibit extended activity and/or super-stoichiometric clearance of a target in a biological system. The conjugates described herein facilitate transport of a target molecule into a cell and may facilitate sequestration and/or degradation of a target molecule of interest in a cell's lysosome.

Also provided herein are compositions comprising such conjugates and methods of using the conjugates to target a polypeptide or molecule of interest for sequestration and/or lysosomal degradation, and methods of using the conjugates.

### BRIEF DESCRIPTION OF THE DRAWINGS

These and other features, aspects, and advantages of the present invention will become better understood with regard to the following description, and accompanying drawings:

FIG. 1A is a schematic diagram of mechanisms that are hypothesized to occur after the administration of conjugates described herein, wherein externalization of the conjugate from the cell is mediated by binding to FcRn.

FIG. 1B is a schematic diagram of mechanisms that are hypothesized to occur after the administration of conjugates described herein, wherein externalization of the conjugate from the cell is mediated by sustained binding to a lysosomal targeting molecule.

FIG. 2 shows a graph of human IgE (hIgE) concentration in serum versus time following administration of Oma, or the Oma conjugate Oma-1226 in mice treated 24 hours prior with hIgE.

FIG. 3 shows a graph of hIgE concentration in mouse serum versus time following administration of hIgE in mice treated 24 hours prior with Oma, or Oma-1226.

FIG. 4 shows a graph of hIgE concentration in mouse serum versus time following administration of hIgE in mice either untreated or treated 24 hours prior with Oma, or Oma-1228.

FIG. 5 shows a graph of hIgE concentration in mouse serum versus time following administration of a super-stoichiometric quantity of hIgE in mice that were either untreated or treated 24 hours prior with Oma, or Oma-L443C-1117.

FIG. 6 shows a graph of hIgE concentration in mouse serum versus time following administration of a super-stoichiometric quantity of hIgE in mice that were either untreated or treated 24 hours prior with Oma or Oma-1119 (DAR4 lysine conjugate).

FIG. 7 shows a graph of hIgE concentration in mouse serum versus time following administration of two doses administration of hIgE in mice that were either untreated or previously treated with Oma-L443C or Oma-L443C-1117.

FIG. 8A-8B show graphs of hIgE concentration in serum versus time following administration of IgE in FcRn wild-type mice (FIG. 8A) or FcRn knockout mice (FIG. 8B) either untreated or previously treated with Oma-L443C, or Oma-L443C-1120.

FIG. 9 demonstrates the cycling of various ASGPR-based conjugates.

FIG. 10 demonstrates the cycling of various M6PR-based conjugates.

FIG. 11 demonstrates the cycling mechanism via FcRn.

FIG. 12 shows a graph of percent ratio of hIgE concentration to starting hIgE concentration in myeloma-transplanted mouse serum versus time following administration of Oma S35H/Y57H-2328 and Oma S35H/Y57H-2327.

FIG. 13 shows a graph of hC5 concentration in mouse serum versus time following administration of hC5, and a subsequent administration of Ecu-2405, Ecu-1117, ALXN1210-2405, and ALXN1210-1117.

### DETAILED DESCRIPTION

### Target Binding Conjugates

This disclosure provides a class of conjugate compounds that include a ligand moiety, X, that binds to a lysosomal targeting molecule conjugated to target-binding moiety, Y, that binds and an antibody that specifically binds to a cell surface target molecule or extracellular target molecule. In some embodiments, binding of the ligand moiety to the lysosomal targeting molecule can trigger internalization of the lysosomal targeting molecule and conjugate. In some embodiments, the antibody conjugate is configured to exhibit extended activity and/or super-stoichiometric clearance of a target in a biological system. “Super-stoichiometric clearance” refers to conjugates facilitating a greater than stoichiometric ratio of target molecule removed from the extracellular environment to the amount of conjugate administered, i.e. where the conjugate facilitates degradation of a molar excess of target relative to the conjugate. The compound or conjugate is capable of binding a target molecule extracellularly, internalizing the target molecule, and releasing the target molecule such that the target molecule is destroyed in the lysosome, and the compound or conjugate is externalized and capable of repeating the process. As such, the ratio of compound or conjugate administered is less than the amount of target molecule cleared or removed from the extracellular environment.

In one embodiment, is provided a ligand moiety, X, conjugated via a linker, L, to a target-binding moiety, Y, wherein the ligand moiety, X, binds a lysosomal targeting molecule extracellularly; the target-binding moiety, Y, binds a target molecule extracellularly; the target-binding moiety, Y, dissociates from the target molecular intraendosomally and the conjugate is externalized from a cell. In some embodiments, Y is an antibody or an antibody fragment.

In some embodiments, the target binding conjugate is of formula (I′):

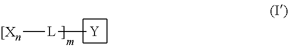

or a prodrug thereof, or a pharmaceutically acceptable salt thereof, wherein:
X is a ligand moiety that binds to a lysosomal targeting molecule;
n is 1 to 20;
L is a linker;
m is 1 to 10; and
Y is an antibody or antibody fragment that specifically binds a cell surface target molecule or extracellular target molecule;
wherein the conjugate is configured to increase duration of the conjugate in active form.

In some embodiments, the conjugate is configured to facilitate degradation of the target molecule for at least one day, or at least two days, or at least four days or at least seven days after administration of the conjugate.

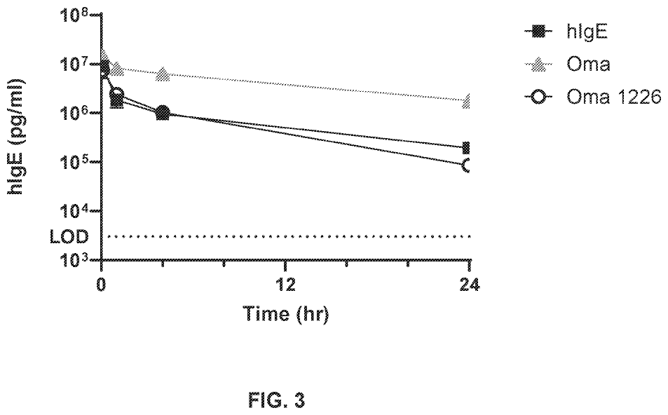

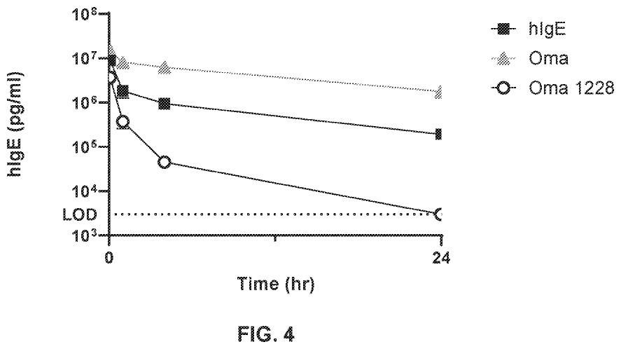

Previously reported lysosome targeting chimeras based on an O-linked GalNac structure (see, e.g. G. Ahn et al., Nat. Chem. Biol. 2021, 17(9) 937-46), while capable of facilitating lysosomal degradation of a target molecule, are not chemically or enzymatically stable in an endosomal environment and are rapidly degraded. In contrast, the conjugates described herein are stable in an endosomal environment as evidenced by, e.g., their ability to facilitate degradation of a target for over 24 hours. Compare the results shown in FIGS. 3 and 4, discussed below.

It is contemplated that conjugates described herein exhibit extended activity or super-stoichiometric clearance of a target in a biological system, or both. In some embodiments, the loading and/or stability of the antibody conjugates can be provided for via use of a conjugation chemistry to a specific site on the antibody, e.g., a cysteine-reactive chemoselective conjugation chemistry.

In some embodiments the ligand is M6PR or ASGPR.

In some embodiments of Formula (I′), n is 1 to 10, such as 1 to 6, 1 to 4, or 1 to 3. In some embodiments, X is a M6PR ligand moiety, and n is 1 to 6, such as n is about 4 (i.e., an average loading of about 4).

In some embodiments, X is a ASGPR ligand moiety, and n is 1 to 3, such as 1 to 2. In some embodiments, n is 1. In some embodiments, n is 2.

In some embodiments of Formula (I′), m is 1 to 6, or 1 to 4. It is understood that depending on the conjugation chemistry, m can refer to a discrete number, or m can refer to an average, e.g., loading of ligand-linker on the antibody (i.e. a DAR ratio).

In some embodiments, Y is selected from a human antibody, humanized antibody, or chimeric antibody. The Y antibody can have a Fc region or Fc domain that provides for binding to FcRn (neonatal fragment crystallizable receptor) in a target cell of interest to facilitate the internalizing and/or degradation activity of the conjugate in the cell. In some embodiments, the Fc region of the antibody in the conjugates of this disclosure includes one or more mutations that impart increased binding affinity for FcRn as compared to a reference Fc region lacking the one or more mutations.

In some embodiments, the antibody Y is selected to have a binding affinity for the target molecule that is calcium (Ca²⁺) dependent or pH dependent, e.g., to facilitate release of the conjugate from the molecule in the cell. In some embodiments, the antibody Y is selected to have a high binding affinity for the cell surface target molecule or extracellular target molecule at neutral pH than at low pH.

In some embodiments of formula (I), the conjugate is of formula (II):

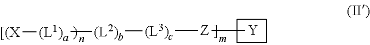

wherein
L¹ and L³ are independently a linker (e.g., as described herein), and L² is a branched linking moiety (e.g., as described herein), wherein L¹ to L³ together provide a linear or branched linker between X and Y;
a, b and c are independently 0 or 1; and
Z is a residual moiety resulting from the covalent linkage of a chemoselective ligation group to a compatible group of Y (e.g., as described herein), wherein:
when n is 1, a is 1, and b is 0;
when n is >1, a is 1, and b is 1.

In some embodiments target binding conjugate is of formula (IIa′):

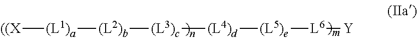

or a prodrug thereof, or a salt thereof,
wherein:
n is 1 to 3;
m is 1 to 3;
X and Y are each independently as defined herein;
each L¹ to L⁶ is independently a linking moiety which together provide a linear or branched linker between Z¹ and Y; and
   a, b, c, d, and e are each independently 1, 2, 3, 4, or 5.

### Lysosomal Targeting Molecules and Ligand Moieties

A lysosomal targeting molecule is a cell surface receptor that provides for internalization of the conjugate compounds of this disclosure.

The term “ligand moiety” refers to a portion of the conjugates described herein that bind to a lysosomal targeting molecule.

In some embodiments, the lysosomal targeting molecule is selected from asialoglycoprotein receptor (ASGPR), cation independent mannose-6-phosphate receptor (CI-M6PR also referred to herein as M6PR), folate receptor, CD63, sortilin, IFITM3, molecules in the endosome/lysosome pathway, LIMP-1, and LIMP-2. In some embodiments, the lysosomal targeting molecule is ASGPR. In some embodiments, the lysosomal targeting molecule is CI-M6PR. In some embodiments, the lysosomal targeting molecule is folate receptor.

A variety of ligand moieties (compounds or moieties that bind to a lysosomal targeting molecule) can be utilized in the conjugate compounds of this disclosure.

In some embodiments, the ligand moiety comprises a monosaccharide. In some embodiments, the ligand moiety comprises a galactose. In some embodiments, the ligand moiety comprises a mannose. In some embodiments, the ligand moiety comprises a pyranose. In some embodiments, the ligand moiety is not an antibody or antibody fragment.

Ligand moieties that bind to ASGPR are described in International Publication WO2023/288033, filed Jul. 14, 2022, the disclosure of which is herein incorporated by reference in its entirety.

Ligand moieties that bind to CI-M6PR are described in International Publication WO2023/288015, filed Jul. 14, 2022, the disclosure of which is herein incorporated by reference in its entirety.

Ligand moieties that bind to folate receptor are described in International Publication WO2022/150721, filed Jan. 10, 2022, the disclosure of which is herein incorporated by reference in its entirety.

In some embodiments of any of the conjugates disclosed herein, when the targeting moiety is bonded to the linker (L) at the C1 carbon atom of X (i.e., the anomeric carbon, such as via R¹ of the ASGPR targeting moieties), then the atom directly bonded to the C1 carbon atom of X is not —O—.

### ASGPR Ligand Moieties

This disclosure provides a class of compounds including a ligand moiety that specifically binds to ASGPR. The ASGPR ligand moieties of this disclosure can be linked to a variety of moieties of interest without impacting the specific binding to, and function of, the cell surface ASGPR. The inventors have demonstrated that compounds of this disclosure can utilize the functions of cell surface ASGPRs in a biological system, e.g., for internalization and sequestration of a compound to the lysosome of a cell, and in some cases subsequent lysosomal degradation. The compounds of this disclosure find use in a variety of applications.

The term “asialoglycoprotein receptor” (ASGPR), also known as the Ashwell Morell receptor, means the transmembrane glycoprotein receptor found primarily in hepatocytes which plays an important role in serum glycoprotein homeostasis by mediating the endocytosis and lysosomal degradation of glycoproteins with exposed terminal galactose or N-acetylgalactosamine (GalNAc) residues. ASGPR cycles between endosomes and the cell surface. In particular embodiments, the ASGPR is Homo sapiens asialoglycoprotein receptor 1 (ASGR1) (see, e.g., NCBI Reference Sequence: NM_001197216).

A compound or moiety or conjugate comprising such ASGPR binding moiety (X) (e.g., as described herein), may bind to other receptors, for example, may bind with lower affinity as determined by, e.g., immunoassays or other assays known in the art. In one embodiment, X, or a compound or moiety or conjugate as described herein comprising such X specifically binds to the cell surface ASGPR with an affinity that is at least 2 logs, 2.5 logs, 3 logs, 4 logs or greater than the affinity when X or the compound or the conjugate bind to another cell surface receptor. In one embodiment, X or a compound or moiety as described herein comprising X, specifically binds to ASGPR with an affinity (K_{d}) 20 mM or less. In some embodiments, such binding is with an affinity (K_{d}) is 10 mM or less, 1 mM or less, 100 uM or less, 10 uM or less, 1 uM or less, 100 nM or less, 10 nM or less, or 1 nM or less. The terms “binds,” “binds to,” “specifically binds” or “specifically binds to” in this context are used interchangeably.

The ASGPR binding compounds or ASGPR ligand moieties of this disclosure include a moiety (X) that specifically binds to the cell surface receptor ASGPR. The ASGPR binding compounds or ASGPR ligand moieties can be monovalent or multivalent (e.g., bivalent or trivalent or of higher valency), where a monovalent compound includes a single ASGPR ligand moiety, and a multivalent compound includes two or more such moieties.

In certain embodiments, the ASGPR binding moiety or ASGPR ligand moiety, X, is able to bind to a specific cell surface ASGPR, and direct (or target) the molecule to this receptor. In certain embodiments, the ASGPR binding moiety or ASGPR ligand moiety, X, is capable of binding to the ASGPR and directing (or targeting) a compound or conjugate described herein for internalization and sequestration to the lysosome, and/or subsequent lysosomal degradation.

In some embodiments, the ASGPR binding moiety or ASGPR ligand moiety, X, includes an amino sugar ring derivative of galactose (e.g., N-Acetylgalactosamine, and analogs thereof), that is linked via a linking moiety to the 1, 6 or 2-position of the sugar ring. The linking moiety can be of 1-10 atoms in length, such as 1-6, or 1-5, 1-4, or 1-3 atoms in length. In some embodiments, the amino sugar ring derivative of galactose is linked via a linking moiety to an oxygen, sulfur, nitrogen or carbon atom the 1-position of the ring. In some embodiments, the amino sugar ring derivative of galactose is linked via a linking moiety to an oxygen, sulfur, nitrogen or carbon atom the 6-position of the ring. In some embodiments, the amino sugar ring derivative of galactose is linked via a linking moiety to an oxygen, sulfur, nitrogen or carbon atom the 2-position of the ring. In certain embodiments, the amino sugar derivative of galactose is linked via a linking moiety to a heteroaryl group at the 1, 6 or 2 position of the ring. In certain embodiments, the amino sugar derivative of galactose is a bicyclic structure.

In some embodiments, the ASGPR binding compounds or ASGPR ligand moiety is monovalent (e.g., in Formula (I), n is 1), such that the ASGPR binding compound or ASGPR ligand moiety includes a single ASGPR ligand moiety (X) that is linked to a moiety of interest or target-binding moiety (Y) via a linking moiety, L, at the 1, 6, or 2-position of (X). In certain embodiments of formula (I), n is 1, and L comprises a linear linker having a backbone of 20 or more consecutive atoms covalently linking the ASGPR ligand (X) to Y via a linking moiety at any of the 1, 2 or 6-positions of X. In certain cases, n is 20 to 100 consecutive atoms, such as 25 to 80, 25 to 60, or 25 to 50. In certain embodiments of formula (I), n is 1, and L comprises a backbone of 25 or more consecutive atoms covalently linking the ASGPR ligand moiety (X) to Y.

In some embodiments, the ASGPR binding compounds are multivalent (e.g., in Formula (I), n is 2 or more, such that the ASGPR binding compound includes two or more ASGPR ligand binding moieties (X) that are each covalently linked to a moiety of interest (Y) via a branched linker (e.g., L is a branched linker). In some embodiments, the ASGPR binding compound is divalent (e.g., n is 2 in Formula 1). In some embodiments, the ASGPR binding compound is trivalent (e.g., n is 3 in Formula 1). In some embodiments, each branch of the branched linker comprises a liner linker of 14 or more consecutive atoms to covalently link a linking moiety of each X to a branching point in the linker. In some embodiments, each branch of the linker includes 14 to 50 consecutive atoms, such as 14 to 40, 14 to 30, or 14 to 20 atoms. In some embodiments, each branch of the linker includes a linear linker of 20 or more consecutive atoms. In some embodiments, the linker comprises a linear linker of 12 or more consecutive atoms to covalently link the branching point of L to a moiety of interest (Y), such as 15 or more, 20 or more, 30 or more, or even more consecutive atoms to covalently link the branching point of L to Y.

ASGPR ligand moieties which can be adapted for use in the conjugates of this disclosure are described in WO/2023288033, filed Jul. 14, 2022, the disclosure of which is herein incorporated by reference in its entirety. Exemplary ASGPR ligand moieties are described below.

In some embodiments, the ASGPR ligand moieties (e.g., Xₙ-L or (X-L)ₙ of formula (I), and other formulae described herein), of the bifunctional molecule specifically bind to ASGPR with an affinity (K_{d}) of 300 nM or less, such as 100 nM or less, 30 nM or less, 10 nM or less, 3 nM or less, or 1 nM or less. The terms “binds,” “binds to,” “specifically binds” or “specifically binds to” in this context are used interchangeably.

In some embodiments of the conjugates described herein, X is an asialoglycoprotein receptor (ASGPR) binding moiety of formula (II):

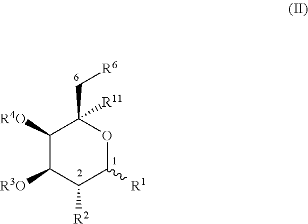

wherein:
R¹ is selected from —Z¹—*, —H, —OH, optionally substituted (C1-C₆)alkyl, —OCH₃, —OCH₂CH═CH, optionally substituted —S—(C₁-C₆)alkyl, optionally substituted aryl, optionally substituted heteroaryl, optionally substituted —S-aryl, and optionally substituted —S-heteroaryl;
R² is selected from —Z¹—*, —NHCOCH₃, —NHCOCF₃, —NHCOCH₂CF₃, —OH, —NHR, and optionally substituted triazole;
R⁶ is selected from —Z¹—*, —OH, —OR, optionally substituted (C₁-C₆)alkyl, —OC(O)R, —C(O)NHR, —NR^{xx}R^{yy}, optionally substituted aryl, optionally substituted heteroaryl, —NHCOR, and —NRCOR;
each R is independently optionally substituted (C₁-C₆)alkyl, optionally substituted aryl, or optionally substituted heteroaryl;
R^{xx} and R^{yy} are independently H, optionally substituted (C₁-C₆)alkyl, or R^{xx} and R^{yy} can cyclize to form an optionally substituted heterocyclyl;
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, —CONR²¹—, —NR²¹SO₂—, —SO₂NR²¹—, —NR²¹C(═O)NR²¹—, and —NR²¹C(═S)NR²¹—;
Z¹¹— is —O—, —S—, —N(R²¹)—, or —C(R²²)₂; provided that when R¹ is-Z¹—*, and Z¹ is —Z¹¹—, then —Z¹¹— is not —O—;
A¹- and -A²- are optionally substituted heterocyclylene, optionally substituted arylene, or optionally substituted heteroarylene;
each R²¹ is independently selected from H, optionally substituted (C₁-C₆)alkyl, —COR, and optionally substituted heteroaryl; and
each R²² is independently selected from H, halogen, and optionally substituted (C₁-C₆)alkyl.

In some embodiments, -L-Y comprises:

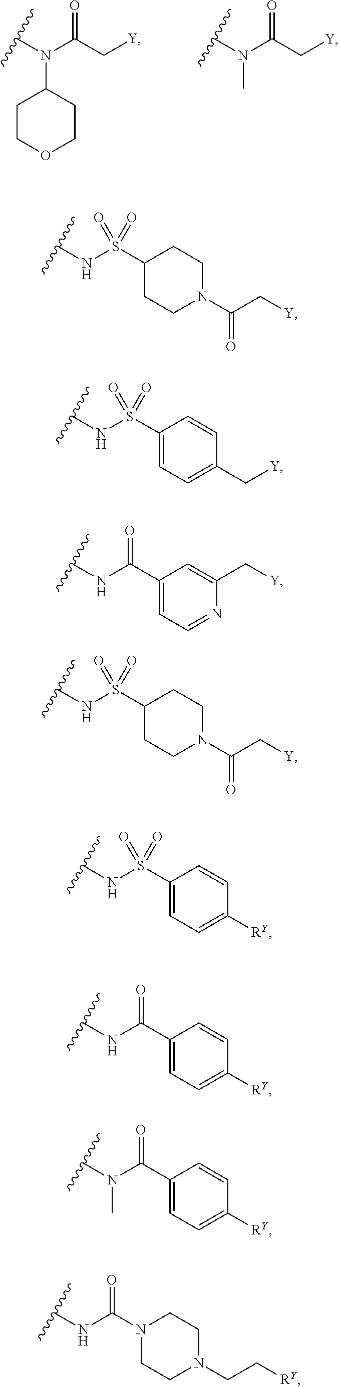

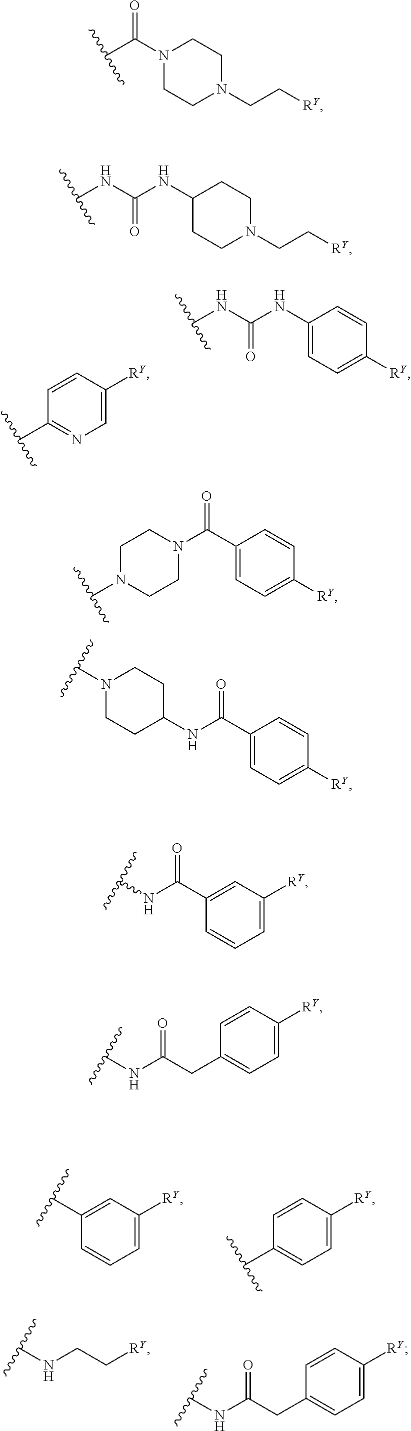

wherein R^{Y} is

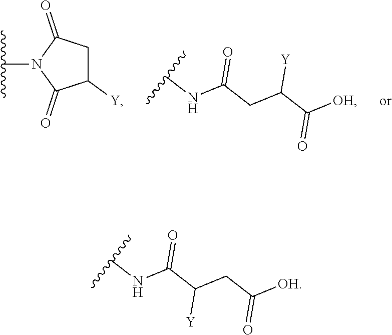

In some embodiments, X is represented by formula (a-II):

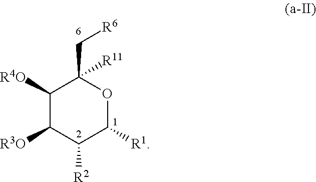

In some embodiments, R¹ is —Z¹—*, —H, or (C₁-C₆)alkyl. In some embodiments, R¹ is —Z¹—*, —H, or n-propyl.

In some embodiments, R² is —Z¹—* or —NHCOCH₃.

In some embodiments, R³ and R⁴ are each —H.

In some embodiments, L comprises of 10 to 60 consecutive branched or linear chain atoms.

In some embodiments, L is of formula (IIb′):

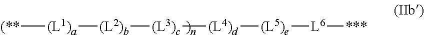

wherein:
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

In some embodiments, L is of formula (IIb′):

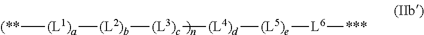

wherein:
n is 1, 2, or 3;
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

In some embodiments, each L¹ to L⁵ independently comprises one or more linking moieties independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50;
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹⁶—, —C₁₋₆-alkylene-NR¹⁶C(S)NR¹⁶—, —O(CH₂)ₚ—, —(OCH₂CH₂)ₚ—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹—; and
each R¹⁶ is independently —H, optionally substituted (C₁-C₆)alkyl, optionally substituted aryl, optionally substituted monocyclic heteroaryl or monocyclic heteroaryl.

In some embodiments, each L¹ to L⁵ is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C( )NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50; and

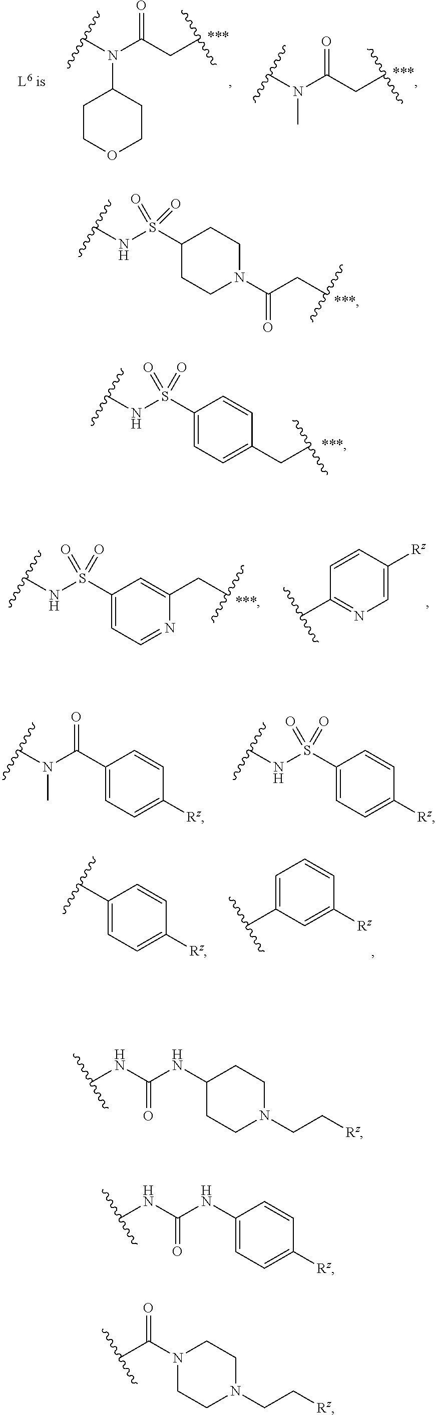

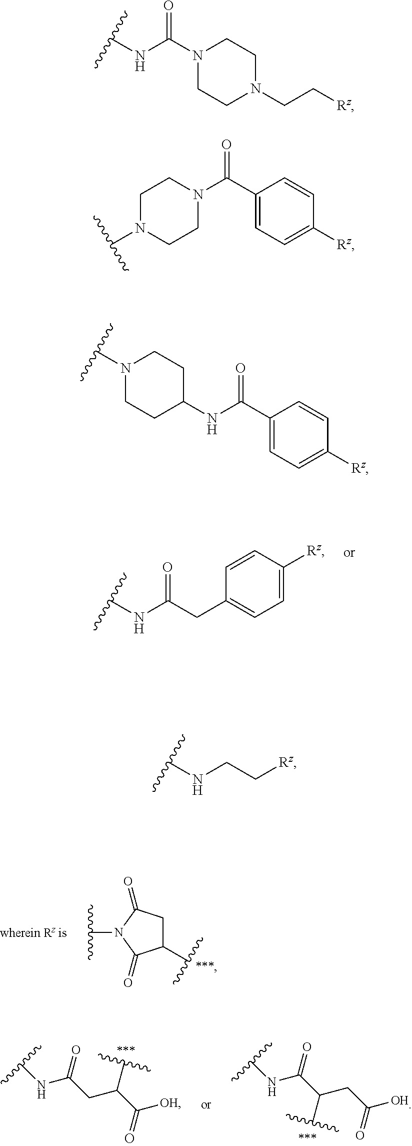

### 1-Linked ASGPR Ligand Moieties

In some embodiments, the ASGPR binding moiety (X) of the compounds of this disclosure can be described by formula (Iia):

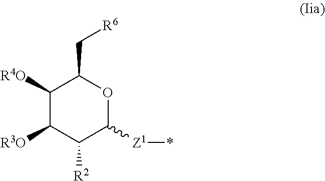

wherein R², R³, R⁴, R and Z¹ are as defined herein. In some embodiments of formula (Iia), R is selected from —OH, —OC(O)R, and —C(O)NHR; and R² is selected from —NHCOCH₃, —NHCOCF₃, and —NHCOCH₂CF₃.

In some embodiments of formula (II), Z¹ is in a beta configuration, and can be described by formula (Iia-1):

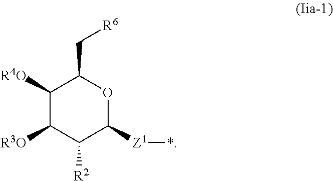

In some embodiments of formula (II), Z¹ is in an alpha configuration, and can be described by formula (Iia-2)

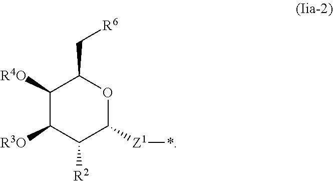

In certain embodiments of formula (Iia), (Iia-1) or (Iia-2), Z¹ is —Z¹¹-A¹-, wherein A¹- is optionally substituted arylene or optionally substituted heteroarylene. In certain embodiments, A¹ is an optionally substituted heteroarylene. In certain embodiments, the heteroarylene is a 5 or 6-membered heteroarylene. In certain embodiments, the heteroarylene is a 5-membered heteroarylene. In certain embodiments, the 5-membered heteroarylene is a triazole. In certain embodiments, the triazole is a 1,2,3-triazole moiety.

In some embodiments, the ASGPR binding moiety (X) of the compounds of this disclosure can be described by formula (IIIa) or (IIIb):

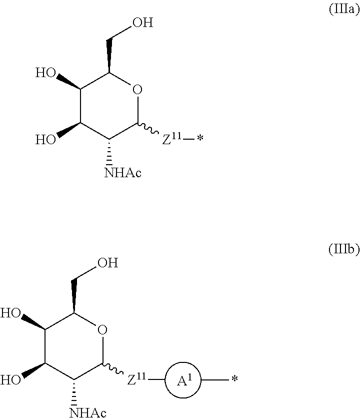

wherein:
Z¹¹— is —O—, —S—, —N(R²¹)—, or —C(R²²)₂—, where each R²² is independently selected from H, halogen (e.g., F) and optionally substituted (C₁-C₆)alkyl, and R²¹ is H or optionally substituted (C₁-C₆)alkyl; and
A¹- is arylene, substituted arylene, heteroarylene, or substituted heteroarylene.

In some embodiments of formula (IIIa) or (IIIb), Z¹¹ is —S—.

In some embodiments, Z¹¹ is —C(R²²)₂—. In some embodiments, Z¹¹ is —CH₂—.

In certain embodiments, Z¹¹ is —C(R²²)₂, where at least one R²² is H. In certain embodiments, both R²² are H. In certain embodiments Z¹¹ is —O—. In certain embodiments, Z¹¹ is —S—. In certain embodiments cases, Z¹¹ is —N(R²¹), where R²¹ is H or (C₁-C₃)alkyl.

In certain embodiments, -A¹- is triazole.

In certain embodiments, Z¹ is —C(R²²)₂-triazole-. In certain embodiments, Z¹ is:

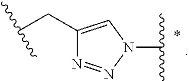 [SMILES: *Cc1cn(*)nn1]

In certain embodiments, Z¹ is:

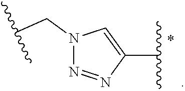 [SMILES: *Cn1cc(*)nn1]

In certain embodiments of formula (Iia), (Iia-1) or (Iia-2), Z¹ is Z¹¹. In certain embodiments, Z¹¹ is —C(R²²)₂. In certain embodiments, at least one R²² is H. In certain embodiments, both R²² are H, and Z¹ is —CH₂—. In certain cases Z¹ is —O—. In certain embodiments, Z¹¹ is —S—. In certain other cases, Z¹¹ is —N(R²¹), where R²¹ is H or (C₁-C₃)alkyl.

In certain embodiments of formula (Iia), (Iia-1) or (Iia-2), Z¹ is monocyclic 5 or 6-membered heteroaryl or aryl. In certain embodiments, Z¹ is

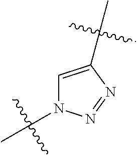 [SMILES: *c1cn(*)nn1 ; "."]

In certain embodiments, Z¹ is

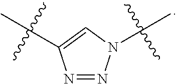 [SMILES: *c1cn(*)nn1]

In certain embodiments of formula (Iia), (Iia-1) or (Iia-2), Z¹ is selected from —O—, —S—, —C(R²²)₂—, —NR²¹—, —CONR²¹—, and

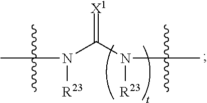

wherein:
X¹ is O or S;
t is 0 or 1;
R²¹ and each R²³ is independently selected from H, and optionally substituted (C₁-C₆)alkyl (e.g., C₍₁₋₃₎-alkyl, such as methyl); and
each R²² is independently selected from H, halogen (e.g., F) and optionally substituted (C₁-C₆)alkyl.

In certain embodiments of formula (Iia), (Iia-1), or (Iia-2), Z¹ is optionally substituted (C₁-C₆)alkyl. In certain embodiments, of Z¹ the alkyl is methyl. In certain embodiments, of Z¹, the alkyl is ethyl. In certain embodiments, of Z¹, the alkyl is propyl. In certain embodiments, of Z¹, the alkyl is butyl. In certain embodiments, of Z¹, the alkyl is pentyl. In certain embodiments, of Z¹, the alkyl is hexyl.

In certain embodiments, the ASGPR binding moiety (X) of formula (Iia-1) is selected from one of the following structures:

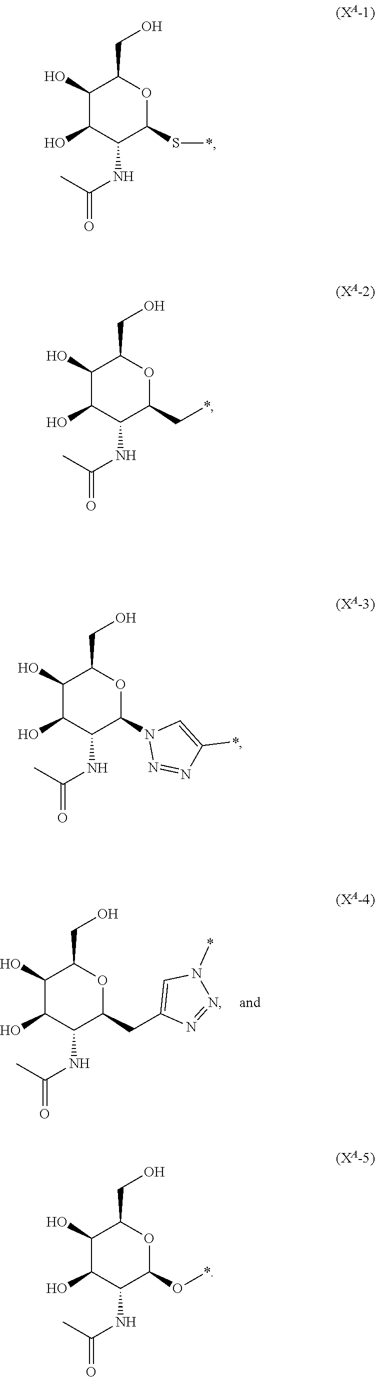

In some embodiments of formula (Iia-2), Z¹ is in a beta configuration and X is of formula (IIIb-2):

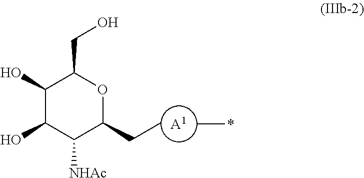

wherein: -A¹- is arylene, substituted arylene, heteroarylene, or substituted heteroarylene.

In some embodiments of formula (IIIb-2), A¹ is a triazole. In some embodiments of formula (IIIb-2), X is of formula (X^{A}-4).

In some embodiments of formula (Iia-1), Z¹ is in a alpha configuration at the 1-position carbon of the galactosamine ring. In some embodiments of formula (Iia-1), Z¹ is S, and each X is of formula (X^{A}-1). In some embodiments of formula (Iia-1), each X is of formula (X^{A}-2). In some embodiments of formula (Iia-1), each X is of formula (X^{A}-3). In some embodiments of formula (Iia-1), each X is of formula (X^{A}-4). In some embodiments of formula (Iia-1), each X is of formula (X^{A}-5).

In certain embodiments, the compound of formula (Iia-2) is selected from one of the following structures:

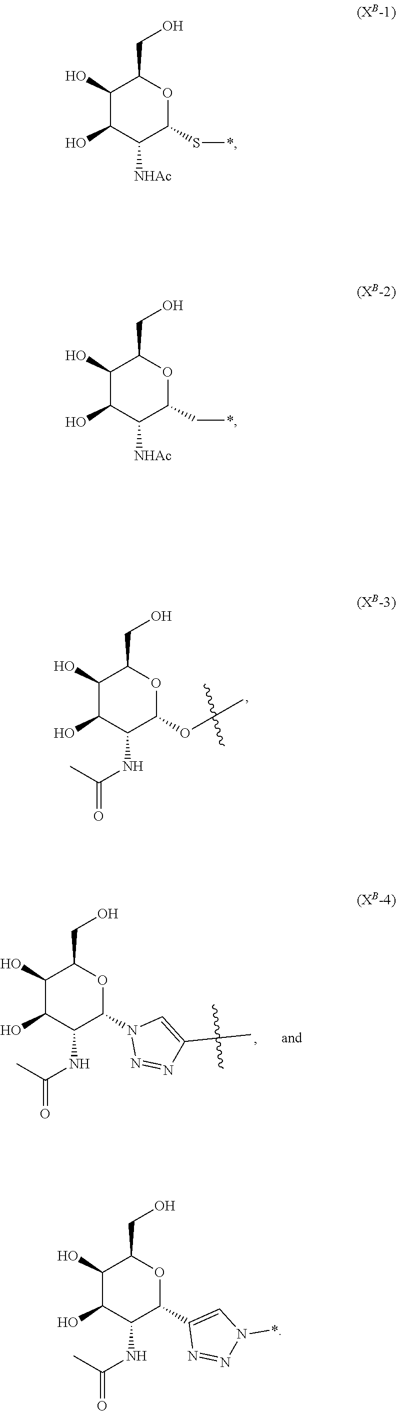

In some embodiments of formula (Iia-2), each X is of formula (X^{B}-1).

In some embodiments of formula (Iia-2), each X is of formula (X^{B}_2).

In some embodiments of formula (Iia-2), each X is of formula (X^{B}-3).

In some embodiments of formula (Iia-2), each X is of formula (X^{B}_4).

In some embodiments of formula (Iia-2), Z¹ is in an alpha configuration and X is of formula (IIIb-1):

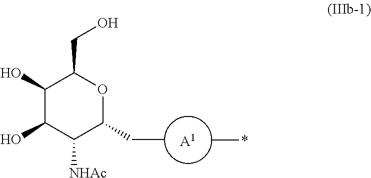

wherein -A¹- is arylene, substituted arylene, heteroarylene, or substituted heteroarylene.

In certain embodiments of formula (IIIb-1), A¹ is an optionally substituted heteroarylene. In certain embodiments, the heteroarylene is a 5 or 6-membered heteroarylene. In certain embodiments, the heteroarylene is a 5-membered heteroarylene. In certain embodiments, the 5-membered heteroarylene is a triazole. In certain embodiments, the triazole is a 1,2,3-triazole moiety.

In certain embodiments, the X of formula (IIIb-1) is selected from one of the following structures:

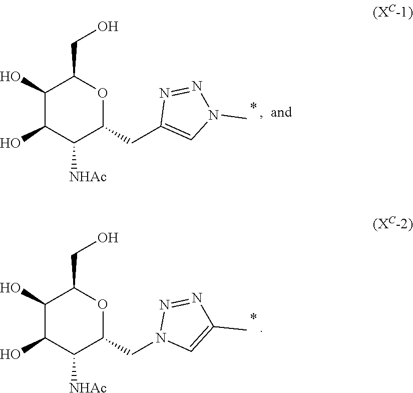

In some embodiments of formula (IIIb-1), each X is of formula (X^{C}-1).

In some embodiments of formula (IIIb-1), each X is of formula (X^{C}-2).

Exemplary ligand moieties that bind ASGPR, and synthons thereof, which can be utilized in the compounds of this disclosure are shown in Tables 1-5. In certain embodiments, the compound of formula (Iia) is a compound shown in Table 1:

TABLE 1
Exemplary ASGPR binding moieties (X) of formula (Iia)

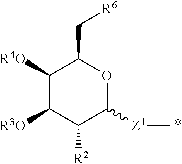

| # | R⁶ | R² | R⁴ | R³ | Z¹ |
|---|---|---|---|---|---|
| X1 | —OH | —NHC(O)CH₃ | H | H | —O— |
| X2 | —OH | —NHC(O)CH₃ | H | H | —S— |
| X3 | —OH | —NHC(O)CH₃ | H | H | —CH₂— |
| X4 | —OH | —NHC(O)CH₃ | H | H | 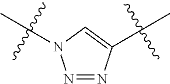 [SMILES: *c1cn(*)nn1] |
| X4.1 | —OH | —NHC(O)CH₃ | H | H | 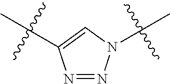 [SMILES: *c1cn(*)nn1] |
| X5 | —OH | —NHC(O)CH₃ | H | —C(O)CH(CH₃)₂ | —O— |
| X5.1 | —OH | —NHC(O)CH₃ | H | H | —NH— |

In some embodiments of any one of X1-A5.1, Z¹ is in the alpha configuration such that the ASGPR binding moiety X1-A5.1 is derived from formula (Iia-2):

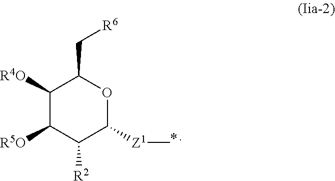

### 2-Linked ASGPR Ligand Moieties

In some embodiments, the ASGPR binding moiety (X) is linked via the 2-position of the sugar analog. In some embodiments, the ASGPR binding moiety (X) has a reduced ring carbon at the 1-position relative to a galactosamine derived sugar. In some embodiments, the ASGPR binding moiety (X) of the bifunctional molecules of this disclosure is described by formula (Iib):

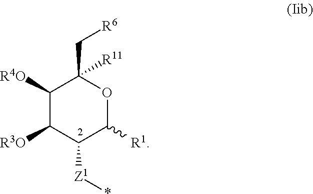

wherein R¹, R³, R⁴, R⁶, R¹¹, and Z¹ are as defined herein.

In some embodiments, the ASGPR binding moiety (X) of the compounds of this disclosure are described by formula (Iib′):

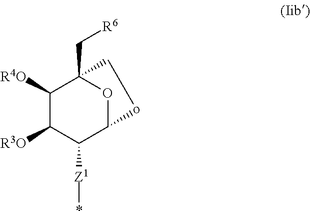

wherein R³-R⁴, R⁶, and Z¹ are as defined herein.

In some embodiments, the ASGPR binding moiety (X) of the compounds of this disclosure are described by formula (Iva):

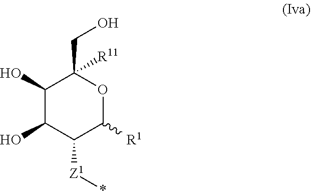

wherein R¹, R¹¹, and Z¹ are as defined herein.

In some embodiments of formulae (Iib), (Iib′) or (Iva), Z1 is selected from optionally substituted —(C(R²²)₂)_{q}-heteroarylene, and

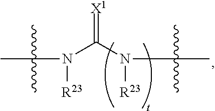

wherein q is 0 or 1.

In some embodiments of formulae (Iib), (Iib′) or (Iva), Z¹ is optionally substituted —(C(R²²)₂)_{q}-triazole wherein q is 0 or 1.

In some embodiments of formulae (Iib), (Iib′) or (Iva), Z¹ is

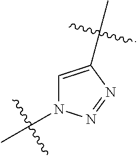 [SMILES: *c1cn(*)nn1 ; "."]

In some embodiments, Z¹ is

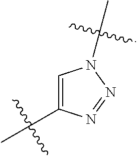 [SMILES: *c1cn(*)nn1 ; "."]

In some embodiments of formulae (Iib), (Iib′) or (Iva), Z¹ is

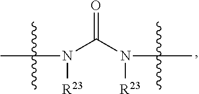

wherein R²³ is H, or C₍₁₋₃₎-alkyl.

In some embodiments of formulae (Iib), (Iib′) or (Iva), Z¹ is —NR²³CO—, wherein R²³ is H or C₍₁₋₃₎-alkyl.

In certain embodiments of formula of formulae (Iib), (Iib′) or (Iva), Z¹ is selected from optionally substituted —(C(R²²)₂)_{q}-heteroaryl, and

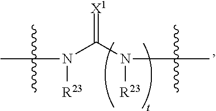

wherein q is 0 or 1.

In certain embodiments of formula of formulae (Iib), (Iib′) or (Iva), Z¹ is optionally substituted —(C(R²²)₂)_{q}-triazole wherein q is 0 or 1. In certain embodiments, Z¹ is

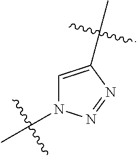 [SMILES: *c1cn(*)nn1 ; "."]

In certain cases of formulae (Iib), (Iib′) or (Iva), Z¹ is

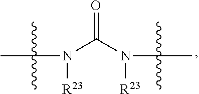

wherein R²³ is H, or C₍₁₋₃₎-alkyl.

In certain cases of formulae (Iib), (Iib′) or (Iva), Z¹ is —NR²³CO—, wherein R²³ is H or C₍₁₋₃₎-alkyl.

In certain embodiments of formula of formulae (Iib), (Iib′) or (Iva), Z¹ is monocyclic 5 or 6-membered heteroarylene or arylene. In certain embodiments, Z¹ is

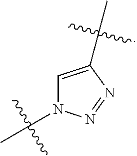 [SMILES: *c1cn(*)nn1 ; "."]

In certain embodiments of formula of formulae (Iib), (Iib′) or (Iva), Z¹ is selected from —O—, —S—, —C(R²²)₂—, —NR²¹—, —CONR²¹—, and

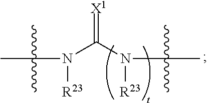

wherein:
X¹ is O or S;
t is 0 or 1;
R²¹ and each R²³ is independently selected from H, and optionally substituted (C₁-C₆)alkyl (e.g., C₍₁₋₃₎-alkyl, such as methyl); and
each R²² is independently selected from H, halogen (e.g., F) and optionally substituted (C₁-C₆)alkyl.

In certain embodiments, the compound of formula of formulae (Iib), (Iib′) or (Iva) is selected from one of the following structures:

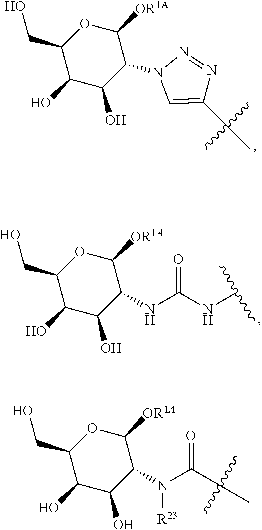

wherein R^{1A} is independently H or (C₁₋₃)alkyl.

In some embodiments, the ASGPR binding moiety (X) of the compounds of this disclosure can be described by formula (Ivb) or (Ivc):

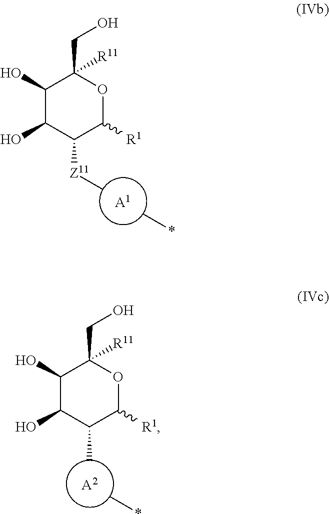

wherein:
Z¹¹— is —O—, —S—, —N(R²¹)—, or —C(R²²)₂;
A¹- and -A²- are optionally substituted arylene or optionally substituted heteroarylene;
each R²¹ is independently selected from H, and optionally substituted (C₁-C₆)alkyl; and
each R²² is independently selected from H, halogen (e.g., F) and optionally substituted (C₁-C₆)alkyl. In some embodiments of formula (Ivb) or (Ivc), R¹ is H.

In some embodiments, R² is —Z¹—*, and R¹¹ is a group of the formula —CH₂O— that forms a bridge (i.e., is cyclically linked) to the 1-position carbon atom on the sugar ring.

In some embodiments, —Z¹—* or —Z¹-L- comprises

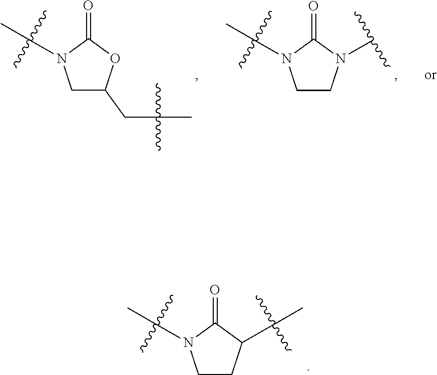 [SMILES: *CC1CN(*)C(=O)O1 ; *N1CCN(*)C1=O ; *C1CCN(*)C1=O]

In certain embodiments of formula (Iib), R¹¹ is H and the compound is of Table 2:

TABLE 2
Exemplary ASGPR binding moieties (X) of formula (Iib)

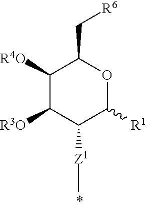

| # | R⁶ | R¹ | R⁴ | R³ | Z¹ |
|---|---|---|---|---|---|
| X9 | —OH | —OCH₃ | H | H | 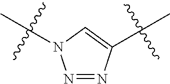 [SMILES: *c1cn(*)nn1] |
| X10 | —OH | —OCH₃ | H | H | —NH(CO)NH— |
| X11 | —OH | —OCH₃ | H | H | —NHC(O)— |

In certain embodiments, the compound of formula (Iib) is a compound shown in Table 3: In certain embodiments, the compound of formula (Jib), the configuration at C1 (i.e., R¹) is alpha. In certain embodiments, the compound of formula (Iib), the configuration at C1 (i.e., R¹) is beta.

TABLE 3
Other exemplary ASGPR binding moieties (X) of formula (Iib)

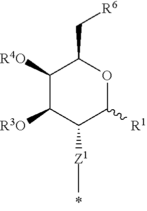

| # | R⁶ | R¹ | R⁴ | R³ | Z¹ |
|---|---|---|---|---|---|
| X12 | —OH | H | H | H | 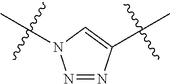 [SMILES: *c1cn(*)nn1] |
| X13 | —OH | H | H | H | 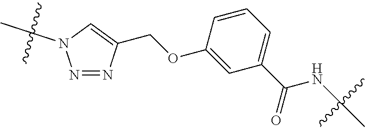 [SMILES: *NC(=O)c1cccc(OCc2cn(*)nn2)c1] |
| X14 | —OH | H | H | H | 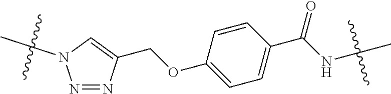 [SMILES: *NC(=O)c1ccc(OCc2cn(*)nn2)cc1] |
| X15 | —OH | H | H | H | —CH₂— |
| X16 | —OH | H | H | H | 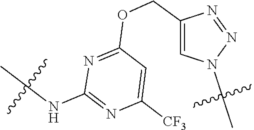 [SMILES: *Nc1nc(OCc2cn(*)nn2)cc(C(F)(F)F)n1] |
| X17 | —OH | H | H | H | 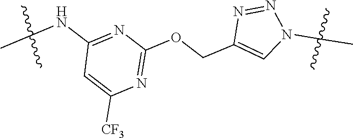 [SMILES: *Nc1cc(C(F)(F)F)nc(OCc2cn(*)nn2)n1] |
| X18 | —OH | H | H | H | 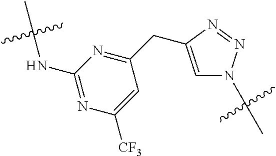 [SMILES: *Nc1nc(Cc2cn(*)nn2)cc(C(F)(F)F)n1] |
| X19 | —OH | H | H | H | 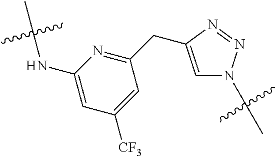 [SMILES: *Nc1cc(C(F)(F)F)cc(Cc2cn(*)nn2)n1] |
| X20 | —OH | H | H | H | 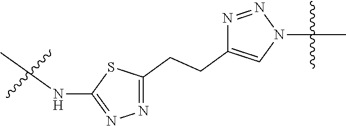 [SMILES: *Nc1nnc(CCc2cn(*)nn2)s1] |
| X21 | —OH | H | H | H | 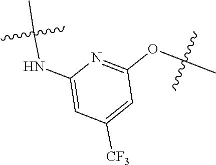 [SMILES: *Nc1cc(C(F)(F)F)cc(O*)n1] |
| X22 | —OH | H | H | H | 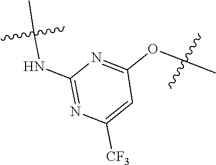 [SMILES: *Nc1nc(O*)cc(C(F)(F)F)n1] |
| X23 | —OH | H | H | H | 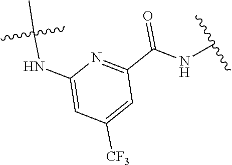 [SMILES: *NC(=O)c1cc(C(F)(F)F)cc(N*)n1] |
| X24 | —OH | H | H | H | —O— |

In certain embodiments, the compound of formula (Id′) is a compound shown in Table 4:

TABLE 4
Other exemplary ASGPR binding moieties (X) of formula (Iib′)

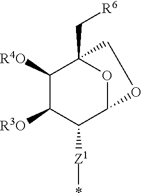

| # | R¹ | R⁴ | R⁵ | Z¹ |
|---|---|---|---|---|
| X25 | —OH | H | H | 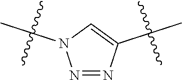 [SMILES: *c1cn(*)nn1] |

In some embodiments, the ASGPR binding moiety (X) of the compounds of this disclosure can be described by formula (Ivb-1) or (Ivc-1):

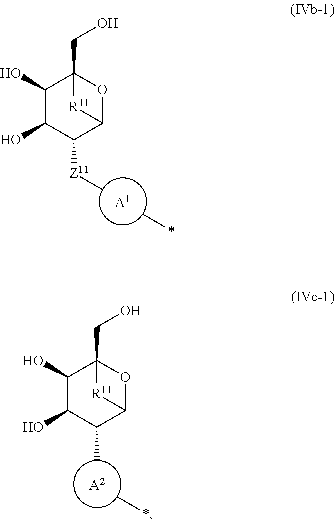

wherein R¹¹ is the bridging moiety that connects the 5-position carbon to the 1-position carbon.

In some embodiments of formulae (Ivb), or (Ivb-1), Z¹¹ is —C(R²²)₂. In certain embodiments, at least one R²² is H. In certain embodiments, both R²² are H. In certain embodiments, Z¹¹ is —O—. In certain embodiments, Z¹¹ is —S—. In certain embodiments, Z¹¹ is —N(R²¹), where R²¹ is H or (C₁-C₃)alkyl.

In certain embodiments of formulae (Ivb), (Ivc), (Ivb-1) or (Ivc-1), -A¹- and -A²- are each independently an optionally substituted heteroarylene. In certain embodiments, the heteroarylene is a 5 or 6-membered heteroarylene. In certain embodiments, the heteroarylene is a 5-membered heteroarylene. In certain embodiments, the heteroarylene is a 6-membered heteroarylene.

In some embodiments of formulae (Ivb), or (Ivb-1), the A¹ ring is a 5-membered heteroarylene selected from triazole, thiadiazole, thiophene, oxazole, isoxazole, isothiazole, thiazole, oxadiazole, and furan. In certain embodiments, the A¹ ring is a 6-membered heteroarylene selected from pyridine, pyrimidine, pyridazine, pyrazine, and triazine. In certain embodiments, the A¹ ring is triazole. In certain embodiments, the A¹ ring is pyridine. In certain embodiments, the A¹ ring is pyrimidine. In certain embodiments, the A¹ ring is thiadiazole. In certain embodiments, the A¹ ring is a 5 or 6-membered arylene or heteroarylene that is further substituted with one or more substituents. In certain embodiments, the A¹ ring is further substituted with one or more substituents selected from halogen, (C₁-C₆)alkyl and substituted (C₁-C₆)alkyl (e.g., CF₃).

In some embodiments of any one of formulae (Ivc), or (Ivc-1), the A² ring is a 5-membered heteroarylene selected from triazole, thiadiazole, thiophene, oxazole, isoxazole, isothiazole, thiazole, oxadiazole, and furan. In certain embodiments, the A² ring is a 6-membered heteroarylene selected from pyridine, pyrimidine, pyridazine, pyrazine, and triazine. In certain embodiments, the A² ring is triazole. In certain embodiments, the A² ring is pyridine. In certain embodiments, the A² ring is pyrimidine. In certain embodiments, the A² ring is thiadiazole. In certain embodiments, the A² ring is a 5 or 6-membered arylene or heteroarylene that is further substituted with one or more substituents. In certain embodiments, the A² ring is further substituted with one or more substituents selected from halogen, (C₁-C₆)alkyl and substituted (C₁-C₆)alkyl (e.g., CF₃).

In certain embodiments of formulae (Ivb) or (Ivb-1), —Z¹¹-A¹- is a monocyclic 5 or 6-membered heteroarylene of one of the following structures:

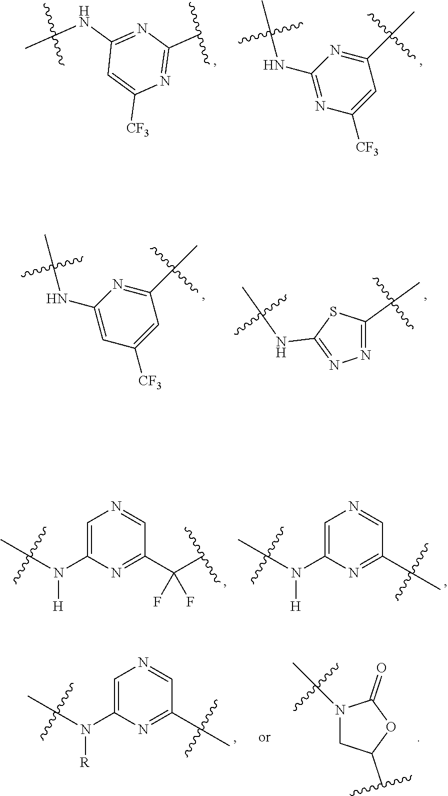

In certain embodiments of formulae (Ivc) or (Ivc-1), -A²- is a monocyclic 5 or 6-membered heteroarylene of the following structure:

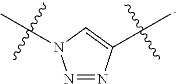 [SMILES: *c1cn(*)nn1]

It is understood that a variety of substituents can be utilized to connect a particular —Z¹¹-A¹- group to an adjacent linker. In certain embodiments of formulae (Ivb) or (Ivb-1), —Z¹¹-A¹- is a monocyclic 5 or 6-membered heteroarylene that is attached to a linking moiety as shown in one of the following structures:

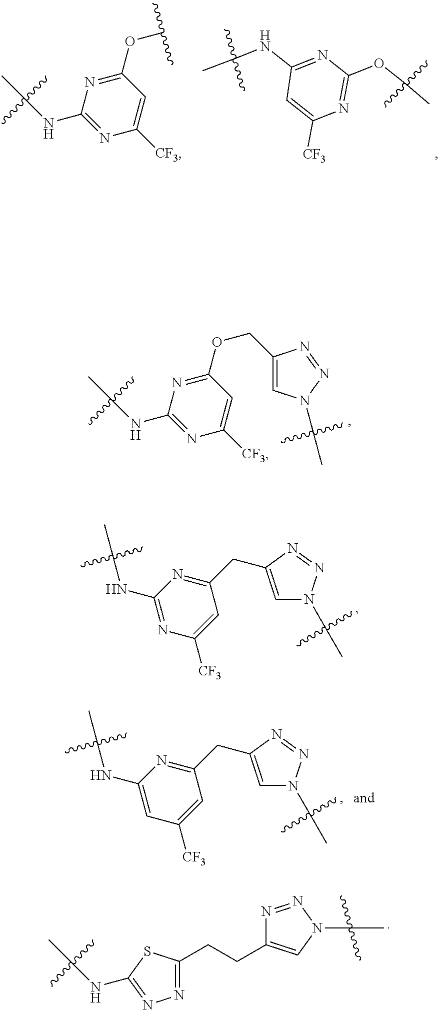

In certain embodiments of formulae (Ivc) or (Ivc-1), —Z¹¹-A¹- is a monocyclic 5 or 6-membered heteroarylene that is attached to a linking moiety as shown in one of the following structures:

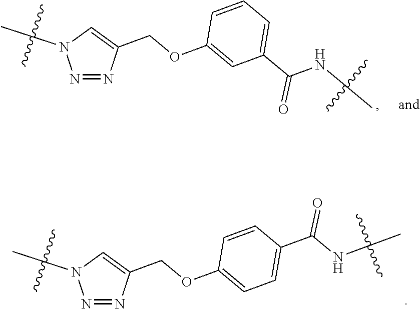 [SMILES: *NC(=O)c1cccc(OCc2cn(*)nn2)c1 ; *NC(=O)c1ccc(OCc2cn(*)nn2)cc1]

In some embodiments of the compound of formula of formulae (Iib), or (Iva)-(Ivc), R¹ is H, such that the compound of formula of formulae (Iib), or (Iva)-(Ivc) has no non-hydrogen substituents at the 1-position of the sugar ring.

In some embodiments, the compound of formula (Iib) is of any one of formulae (Ivd)-(Ivg):

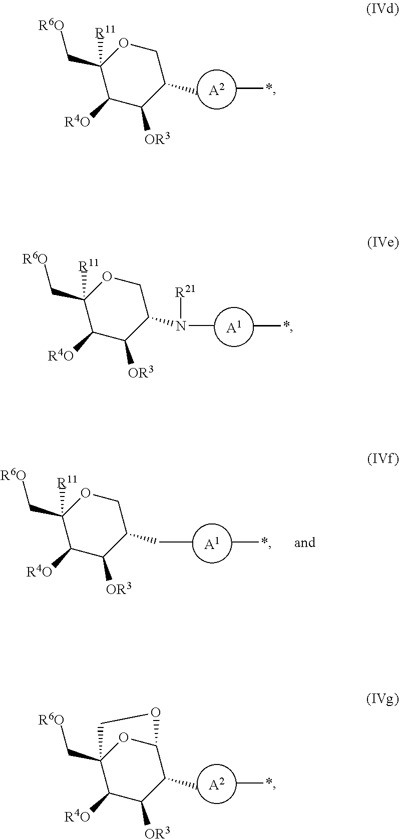

wherein the A¹ and A² rings, R⁶, R⁴, R³, R¹¹, and R²¹ are as defined herein.

In some embodiments of any one of formulae (Ivd)-(Ivg), the A¹ ring is a 5 or 6-membered arylene or heteroarylene. In certain embodiments, the A¹ ring is a 5-membered heteroarylene selected from triazole, thiadiazole, thiophene, oxazole, isoxazole, isothiazole, thiazole, oxadiazole, imidazole, and furan. In certain embodiments, the A¹ ring is a 6-membered heteroarylene selected from pyridine, pyrimidine, pyridazine, pyrazine, and triazine. In certain embodiments, the A¹ ring is triazole. In certain embodiments, the A¹ ring is pyridine. In certain embodiments, the A¹ ring is pyrimidine. In certain embodiments, the A¹ ring is thiadiazole. In certain embodiments, the A¹ ring is pyrazine. In certain embodiments, the A¹ ring is a 5 or 6-membered arylene or heteroarylene that is further substituted with one or more substituents. In certain embodiments, the A¹ ring is further substituted with one or more substituents selected from halogen, (C₁-C₆)alkyl and substituted (C₁-C₆)alkyl (e.g., CF₃).

In some embodiments of any one of formulae (Ivd)-(Ivg), the A² ring is a 5 or 6-membered arylene or heteroarylene. In certain embodiments, the A² ring is a 5-membered heteroarylene selected from triazole, thiadiazole, thiophene, oxazole, isoxazole, isothiazole, thiazole, oxadiazole, and furan. In certain embodiments, the A² ring is a 6-membered heteroarylene selected from pyridine, pyrimidine, pyridazine, pyrazine, and triazine. In certain embodiments, the A² ring is triazole. In certain embodiments, the A² ring is pyridine. In certain embodiments, the A² ring is pyrimidine. In certain embodiments, the A² ring is thiadiazole. In certain embodiments, the A² ring is a 5 or 6-membered arylene or heteroarylene that is further substituted with one or more substituents. In certain embodiments, the A² ring is further substituted with one or more substituents selected from halogen, (C₁-C₆)alkyl and substituted (C₁-C₆)alkyl (e.g., CF₃).

In some embodiments of any one of formulae (Ivd)-(Ivg), the A¹ or A² ring is absent.

In some embodiments of any one of formulae (Ivd)-(Ivg), the A¹ or A² ring is phenylene or substituted phenylene.

In some embodiments of formula (Ivd), the A² ring is a 5 or 6-membered heteroarylene. In certain cases of formula (Ivd), the A² ring is a 5-membered heteroarylene. In certain embodiments of formula (Ivd), the A² ring is triazole. In certain embodiments of (Ivd), the A² ring is absent.

In some embodiments of formula (Ive), the A¹ ring is a 5 or 6-membered heteroarylene and R²¹ is H. In certain embodiments of formula (Ive), the A ring is triazole. In certain cases of formula (Ive), the A¹ ring is pyridine. In certain cases of formula (Ive), the A¹ ring is pyrimidine. In certain cases of formula (Ive), the A¹ ring is thiadiazole. In some embodiments of formula (Ive), the A¹ ring is absent and R²¹ is H or optionally substituted acyl. In certain embodiments, R²¹ is —COCH₃. In certain embodiments, R²¹ is H.

In some embodiments of formula (Ivf), the A¹ ring is a 5 or 6-membered heteroarylene. In certain cases of formula (Ivf), the A¹ ring is a 5-membered heteroarylene. In certain embodiments of formula (Ivf), the A¹ ring is triazole. In certain embodiments of (Ivf), the A¹ ring is absent.

In some embodiments of formula (Ivg), the A² ring is a 5 or 6-membered heteroarylene. In certain cases of formula (Ivg), the A² ring is a 5-membered heteroarylene. In certain embodiments of formula (Ivg), the A² ring is triazole. In certain embodiments of (Ivg), the A² ring is absent.

In certain embodiments, the ASGPR binding moiety (X) of the compounds of this disclosure can be described by any one of formulae (Ivh)-(Ivk):

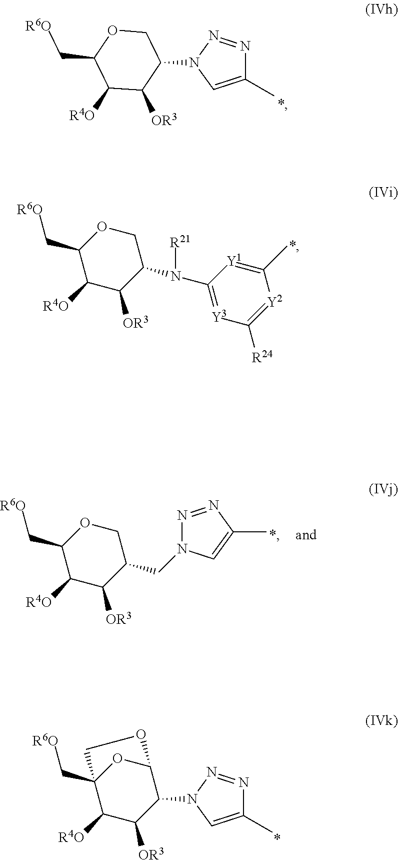

wherein:
R⁶, R⁴, R³, and R²¹ are as defined herein;
Y¹-Y³ are each independently N or CR²⁵; and
R²⁴ and R²⁵ are each independently selected from H, optionally substituted C₍₁₋₆₎-alkyl, optionally substituted fluoroalkyl, and halogen.

In certain embodiments, the ASGPR binding moiety (X) of the compounds of this disclosure can be described by any one of formulae (Ivl)-(Ivm):

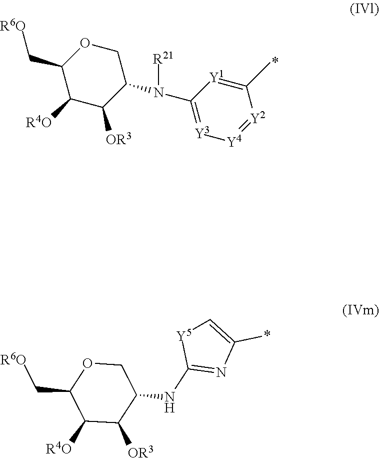

wherein:
R⁶, R⁴, R³, and R²¹ are as defined herein;
Y¹-Y³ are each independently N or CR²⁵;
Y⁴ is N or CR²⁴;
Y⁵ is S, O, or NH; and
R²⁴ and R²⁵ are each independently selected from H, optionally substituted C₍₁₋₆₎-alkyl, optionally substituted fluoroalkyl, and halogen.

In some embodiments of formula (Ivi) at least one of Y¹ to Y³ is N.

In certain embodiments, at least two of Y¹ to Y³ are N.

In certain embodiments, Y¹ and Y⁴ are N.

In certain embodiments, Y¹ and Y³ are N and Y² is CR²⁵.

In certain embodiments, Y¹ and Y² are N and Y³ is CR²⁵.

In certain embodiments, Y¹ and Y² are CR²⁵ and Y³ is N.

In certain embodiments of any one of formulae (Ivd)-(Ivk), or (Ivd)-(Ivm), R⁶ is H.

In some embodiments of any one of formulae (Ivd)-(Ivk), or (Ivd)-(Ivm), R⁴ and R³ are each H. In certain embodiments, at least one of R⁴-R³ is a promoiety. In certain embodiments, R⁴ and R³ are cyclically linked to form a promoiety (e.g., as described herein).

In some embodiments, the compound of formula (Ivi) is of formula (Ivi-1):

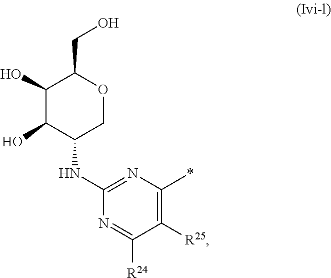

wherein R²⁴ and R²⁵ are independently selected from H, halogen, (C₁-C₆)alkyl and substituted (C₁-C₆)alkyl (e.g., CF₃). In some embodiments of formula (Ivi)-(Ivi-1), R²⁵ is H. In certain embodiments, R²⁵ is C₍₁₋₃₎-alkyl, or C₍₁₋₃₎-fluoroalkyl. In certain embodiments, the fluoroalkyl is CF₃. In some embodiments of formula (Ivi) or (Ivi-1), R²⁴ is H. In certain embodiments, R²⁴ is C₍₁₋₃₎-alkyl, or C₍₁₋₃₎-fluoroalkyl. In certain embodiments, the fluoroalkyl is CF₃.

In some embodiments, the compound of formula (Ivi-1) is of formula (X^{D}):

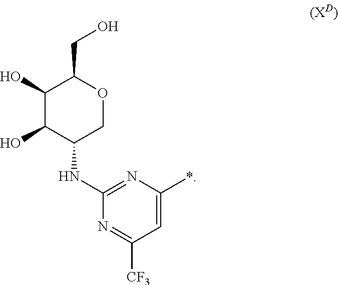

In some embodiments, the compound of formula (Ivk-1) is of formula (X^{E}):

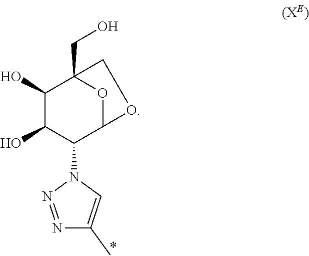 [SMILES: Cc1cn([C@H]2C3OC[C@](CO)(O3)[C@H](O)[C@@H]2O)nn1]

In certain embodiments, the compound of formula (Ivl) is of formula (Ivl-1):

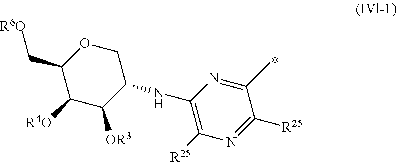

wherein:
R⁶, R⁴, R³, and R²¹ are as defined herein;
Y¹-Y⁴ are each independently N or CR²⁵;
Y⁵ is S, O, or NH; and
each R²⁵ is independently selected from H, optionally substituted C₍₁₋₆₎-alkyl, optionally substituted fluoroalkyl, and halogen.

In certain embodiments, each R²⁵ is H.

In certain embodiments, the ASGPR binding moiety (X) of the compounds of this disclosure can be described by one of the following structures:

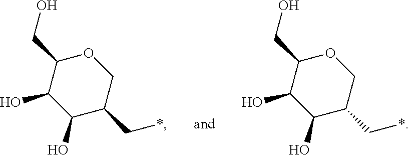

In certain embodiments, the ASGPR binding moiety (X) of the compounds of this disclosure can be described by one of the following structures:

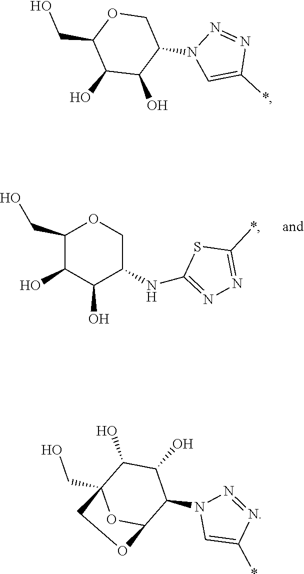

In certain embodiments, the ASGPR binding moiety (X) of the compounds of this disclosure can be described by one of the following structures:

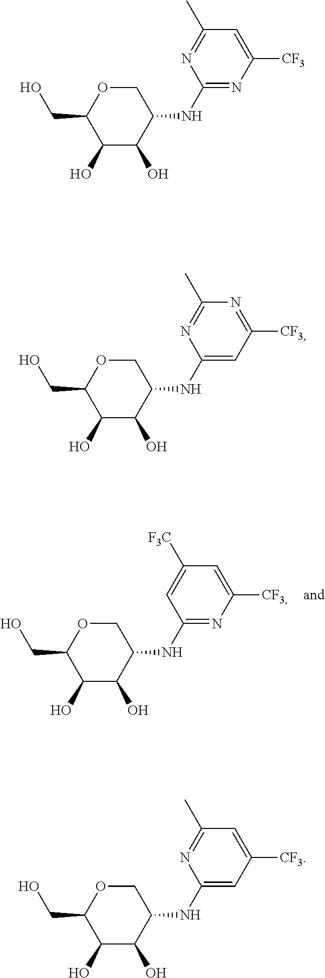 [SMILES: Cc1cc(C(F)(F)F)nc(N[C@H]2CO[C@H](CO)[C@H](O)[C@@H]2O)n1 ; Cc1nc(N[C@H]2CO[C@H](CO)[C@H](O)[C@@H]2O)cc(C(F)(F)F)n1 ; OC[C@H]1OC[C@H](Nc2cc(C(F)(F)F)cc(C(F)(F)F)n2)[C@@H](O)[C@H]1O ; Cc1cc(C(F)(F)F)cc(N[C@H]2CO[C@H](CO)[C@H](O)[C@@H]2O)n1]

In certain embodiments, the ASGPR binding moiety (X) of the compounds of this disclosure can be described by one of the following structures:

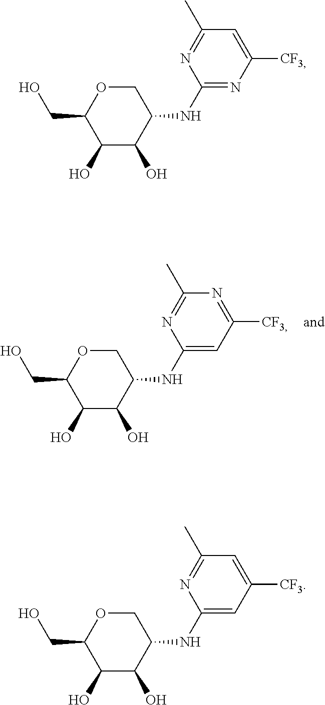 [SMILES: Cc1cc(C(F)(F)F)nc(N[C@H]2CO[C@H](CO)[C@H](O)[C@@H]2O)n1 ; Cc1nc(N[C@H]2CO[C@H](CO)[C@H](O)[C@@H]2O)cc(C(F)(F)F)n1 ; Cc1cc(C(F)(F)F)cc(N[C@H]2CO[C@H](CO)[C@H](O)[C@@H]2O)n1]

In certain embodiments of formula (Iib), R¹ R³, R⁴, and R¹¹ are H, and R⁶ is OH:

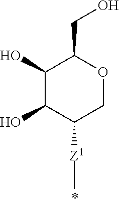

wherein Z¹ is —NH—, —CH₂—, —S— or —O—.

In certain embodiments of formula (Iib′), R³, R⁴ are H, and R⁶ is OH:

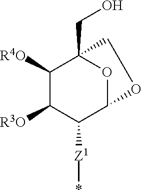

wherein Z¹ is —NH—, —CH₂—, —S—, —O—, triazole, e.g.,

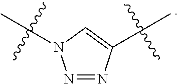 [SMILES: *c1cn(*)nn1]

### 6-Linked ASGPR Ligand Moieties

In some embodiments, the ASGPR binding moiety (X) is linked via the 6-position of the sugar analog. In some embodiments, the ASGPR binding moiety (X) has a reduced ring carbon at the 1-position relative to a galactosamine derived sugar.

In some embodiments, the ASGPR binding moiety (X) of the compounds of this disclosure can be described by formula Iic):

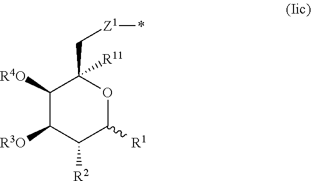

wherein R¹-R⁴ and Z¹ are as defined herein.

In certain embodiments of formula (Iic), Z1 is selected from —O—, —S—, —CONR²¹—, and optionally substituted —(C(R²²)₂)q- heteroarylene, wherein q is 0 or 1. In certain embodiments, Z1 is —O—. In certain other cases, Z1 is optionally substituted —(C(R²²)₂)q-triazole wherein q is 0 or 1. In certain embodiments, Z¹ is

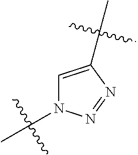 [SMILES: *c1cn(*)nn1 ; "."]

In certain embodiments of formula (Iic), Z¹ is —Z¹¹-A¹-, wherein -A¹- is or optionally substituted -A¹- or optionally substituted arylene. In certain embodiments, -A¹- is an optionally substituted heteroarylene. In certain embodiments, the heteroarylene is a 5 or 6-membered heteroarylene. In certain embodiments, the heteroarylene is a 5-membered heteroarylene. In certain embodiments, the 5-membered heteroarylene is a triazole. In certain embodiments, the triazole is a 1,2,3-triazole moiety. In certain embodiments, Z¹¹ is —C(R²²)₂. In certain embodiments, at least one R²² is H. In certain embodiments, both R²² are H. In certain cases Z¹¹ is —O—. In certain embodiments, Z¹¹ is —S—. In certain other cases, Z¹¹ is —N(R²¹), where R²¹ is H or (C₁-C₃)alkyl. In certain embodiments, Z¹ is —C(R²²)₂-triazole-. In certain embodiments, Z¹ is:

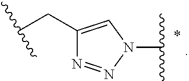 [SMILES: *Cc1cn(*)nn1]

In certain embodiments of formula (Iic), Z¹ is Z¹¹. In certain embodiments, Z¹¹ is —C(R²²)₂. In certain embodiments, at least one R²² is H. In certain embodiments, both R²² are H, and Z¹¹ is —CH₂—. In certain cases Z¹¹ is —O—. In certain embodiments, Z¹¹ is —S—. In certain other cases, Z¹¹ is —N(R²¹), where R²¹ is H or (C₁-C₃)alkyl.

In certain embodiments of formula (Iic), Z¹ is monocyclic 5 or 6-membered heteroarylene or arylene. In certain embodiments, Z¹ is

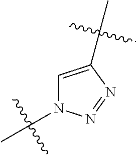 [SMILES: *c1cn(*)nn1 ; "."]

In certain embodiments of formula (Iic), Z1 is selected from —O—, —S—, —C(R²²)₂—, —N(R²¹)—CON(R²¹)—, and

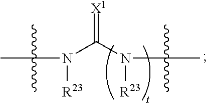

wherein:
X¹ is O or S;
t is 0 or 1;
R²¹ and each R²³ is independently selected from H, and optionally substituted (C₁-C₆)alkyl (e.g., C₍₁₋₃₎-alkyl, such as methyl); and
each R²² is independently selected from H, halogen (e.g., F) and optionally substituted (C₁-C₆)alkyl.

In certain embodiments, the compound of formula (Iic) is the following structure:

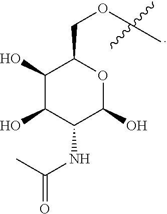 [SMILES: *OC[C@H]1O[C@@H](O)[C@H](NC(C)=O)[C@@H](O)[C@H]1O]

In certain embodiments, the compound of formula (Iic) is the following structure:

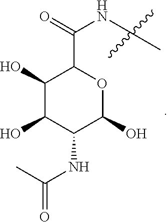 [SMILES: *NC(=O)C1O[C@@H](O)[C@H](NC(C)=O)[C@@H](O)[C@H]1O]

In certain embodiments of formula (Iic), R¹¹ is H and the compound is of Table 5:

TABLE 5
Exemplary ASGPR binding moieties (X) of formula (Iic)

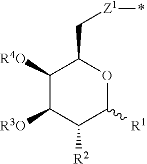

| # | R¹ | R² | R⁴ | R³ | Z¹ |
|---|---|---|---|---|---|
| X6 | —OH | —NHC(O)CH₃ | H | H | —O— |
| X7 | —OCH₃ | —NHC(O)CH₃ | H | H | —C(O)NH— |
| X8 | —OCH₃ | —NHC(O)CH₃ | H | H | —O— |
| | H | —NHC(O)CH₃ | H | H | —NH(O)C— |
| | nPr | —NHC(O)CH₃ | H | H | —NH(O)C— |

In some embodiments, the ASGPR binding moiety (X) of the compounds of this disclosure can be described by formula (Iid):

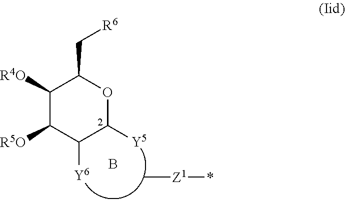

wherein:
R⁶, R⁴, R³ and Z¹ are as defined herein; Y⁶ and Y⁵ are each independently selected from —O—, —S—, NR²¹—, and —C(R²²)₂;
R²¹ is selected from H, optionally substituted (C₁-C₆)alkyl, and —C(O)R²²;
each R²² is independently selected from H, halogen and optionally substituted (C₁-C₆)alkyl; and
ring B is a 5 or 6-membered optionally substituted cyclic group. In some embodiments of formula (Iid), Y⁵ is connected to the sugar ring via an alpha configuration. In some embodiments of formula (Iid), Y⁵ is connected to the sugar ring via a beta configuration.

In some embodiments, the ASGPR binding moiety (X) of the compounds of this disclosure can be described by formula (Iid′):

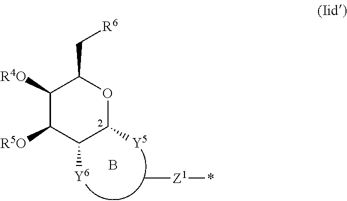

wherein:
R⁶, R⁴, R³ and Z¹ are as defined herein;
Y⁵ and Y⁶ are each independently selected from —O—, —S—, NR²¹—, and —C(R²²)₂;
R²¹ is selected from H, optionally substituted (C₁-C₆)alkyl, and —C(O)R²²;
each R²² is independently selected from H, halogen and optionally substituted (C₁-C₆)alkyl; and
ring B is a 5 or 6-membered optionally substituted cyclic group.

In some embodiments of formula (Iid)-(Iid′) Y⁵ is O. In certain embodiments, Y⁵ is S. In certain embodiments, Y⁵ is —NR²¹—. In certain embodiments, Y⁵ is —C(R²²)₂ and each R²² is H.

In some embodiments of formula (Iid)-(Iid′) Y⁶ is —NR²¹— where R²¹ is H. In certain embodiments, Y⁶ is —NR²¹— where R²¹ is —C(O)R²². In certain embodiments, R²² is methyl.

In some embodiments of formula (Iid)-(Iid′) the B ring is a 5 or 6-membered heterocycle. In certain embodiments, the B ring is a 5-membered heterocycle. In certain embodiments, the B ring is a 6-membered heterocycle.

In some embodiments of formula (Iid)-(Iid′) Z¹ is Z¹¹, where Z¹¹ is selected from —O—, —S—, NR²¹—, and —C(R²²)₂. In certain embodiments, Z¹ is —O—. In certain embodiments, Z¹ is —S—. In certain embodiments, Z¹ is NR²¹ where R²¹ is H. In certain embodiments, Z¹ is —C(R²²)₂ where each R²² is H.

In some embodiments of formula (Iid)-(Iid′) Z¹ is optionally substituted Z¹¹-heteroarylene or optionally substituted Z¹¹-arylene. In some embodiments, Z¹ is CH₂-heteroarylene or CH₂-arylene. In some embodiments of formula (Iid)-(Iid′) Z¹ is optionally substituted amide. In some embodiments of formula (Iid)-(Iid′) Z¹ is optionally substituted sulfonamide. In some embodiments of formula (Iid)-(Iid′) Z¹ is optionally substituted urea or optionally substituted thiourea.

In some embodiments, the compound of formula (Iid)-(Iid′) has one of the following structures:

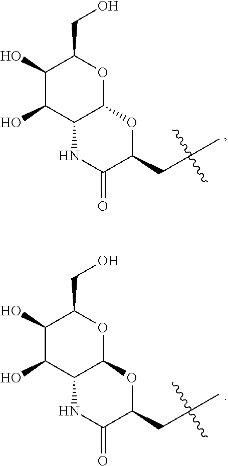 [SMILES: *C[C@@H]1O[C@H]2O[C@H](CO)[C@H](O)[C@H](O)[C@H]2NC1=O ; *C[C@@H]1O[C@@H]2O[C@H](CO)[C@H](O)[C@H](O)[C@H]2NC1=O]

In certain embodiments of any one of formulae (Iia), (Iib) or (Iid), R⁶ is OH. In certain other cases, R⁶ is —OC(O)R. In certain embodiments, R⁶ is —C(O)NHR, where R is an optionally substituted alkyl. In certain embodiments, R terminates in an alkenyl or an alkynyl group. In certain other cases R⁶ is optionally substituted triazole. In certain embodiments, the triazole is of the following structure:

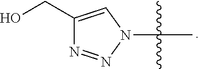 [SMILES: *n1cc(CO)nn1]

In certain embodiments of (Iia), and (Iic), R² is —NHCOCH₃. In certain other embodiments, R² is —NHCOCF₃. In certain other embodiments, R² is —NHCOCH₂CF₃. In certain embodiments, R² is —OH. In certain other cases, R² is an optionally substituted triazole. In certain embodiments, the triazole in of the following structure:

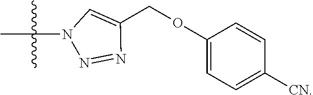 [SMILES: *n1cc(COc2ccc(C#N)cc2)nn1]

In certain embodiments when Rt or R² is a substituted triazole, the triazole is a 1,2,3-triazole, and the substituent is at the 4 or 5-position. In certain embodiments, the substituent on the triazole moiety includes but is not limited to, an optionally substituted (C₁₋₆)alkyl, optionally substituted (C₁₋₆)alkoxy, optionally substituted aryl, optionally substituted heteroaryl, optionally substituted alkaryl, and an optionally substituted alkylheteroaryl. It will be understood that any convenient substituent can be included in the triazole moiety, see, e.g., triazole moieties disclosed in Mamidayala et al, J. Am. Chem. Soc. 2012, 134, 1978-1981.

It is understood that the Z¹, Z¹¹, and Z¹¹—Ar linking moieties can be considered part of the X group of formula (I). In the ASGPR binding moieties (X) as described herein, —Z¹— can be linked to an -L¹- moiety (e.g., of the linker as described herein) via a variety of bonds and linking moieties, depending on the method of preparation. In some embodiments, the subject compounds comprise a —Z¹-L¹- moiety selected from:

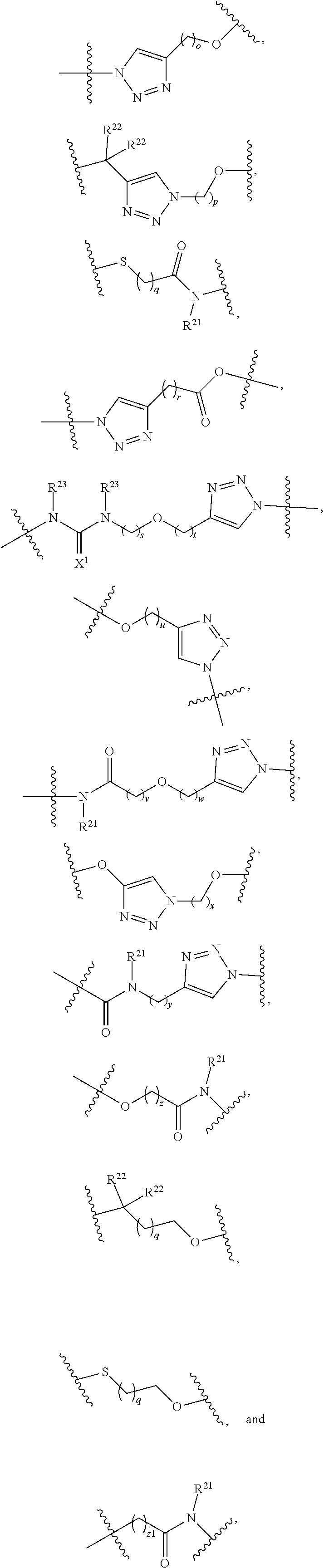

wherein each R²¹ is independently selected from H, and optionally substituted (C₁-C₆)alkyl; each R²² is independently selected from H, halogen (e.g., F) and optionally substituted (C₁-C₆)alkyl; and o, p, q, r, s, t, u, v, w, x, y, z and z1 are each independently 0 to 6.

In some embodiments, the subject compounds comprise a —Z¹-L¹- moiety selected from:

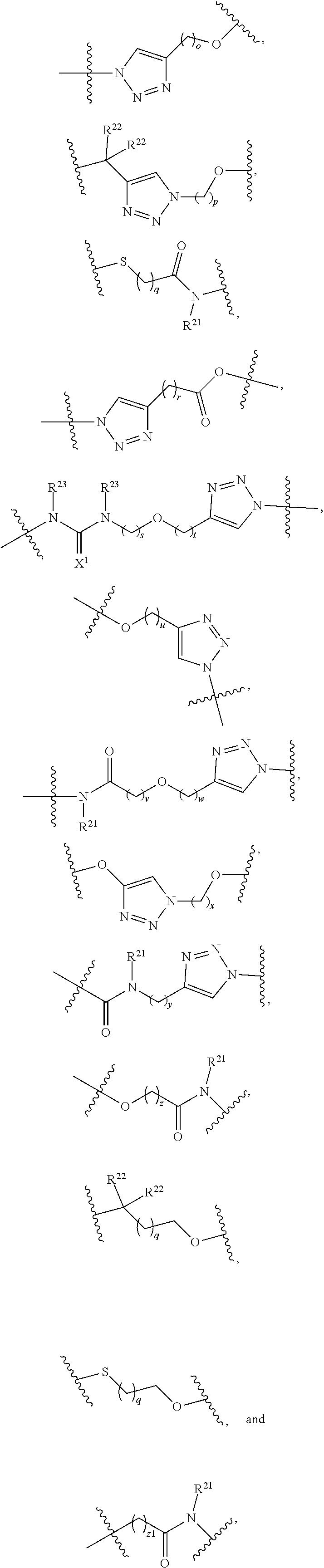

wherein each R²¹ is independently selected from H, and optionally substituted (C₁-C₆)alkyl; each R²² is independently selected from H, halogen (e.g., F) and optionally substituted (C₁-C₆)alkyl; and o, p, q, r, s, t, u, v, w, x, y, z and z1 are each independently 1 to 6.

In certain embodiments, the Z¹-L¹- group is

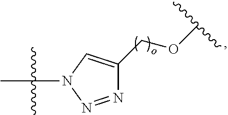

and o is 1 or 2.

In certain embodiments, the Z¹-L¹- group is

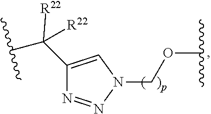

each R²² is H, and p is 1 or 2.

In certain embodiments, the Z¹-L¹- group is

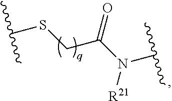

where q is 1-3.

In certain embodiments, the Z¹-L¹- group is

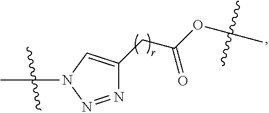

In certain embodiments, the Z¹-L¹- group is

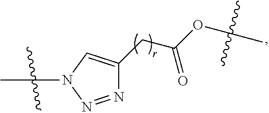

where r is 1-3.

In certain embodiments, the Z¹-L¹- group is

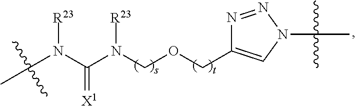

where s and t are each independently 1-3.

In certain embodiments, the Z¹-L¹- group is

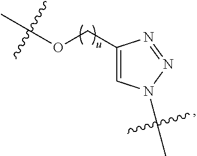

where u is 1-3.

In certain embodiments, the Z¹-L¹- group is

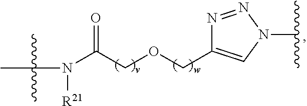

where v and w are each independently is 1-3.

In certain embodiments, the Z¹-L¹- group is

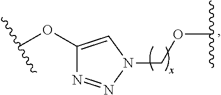

where x is 0-3.

In certain embodiments, the Z¹-L¹- group is

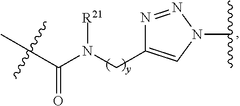

where y is 1-3.

In certain embodiments, the Z¹-L¹- group is

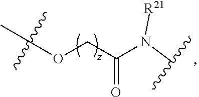

where R²¹ is H, and z is 1-4.

In certain embodiments, the Z¹-L¹- group is

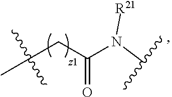

where R²¹ is H, and z1 is 1-4.

In certain embodiments, the Z¹-L¹- group is

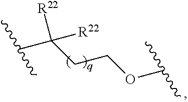

where each R²² is H, and q is 0-3. In certain embodiments, the Z¹-L¹-group is

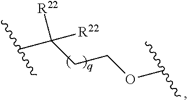

where each R²² is H, and q is 1-3.

In certain embodiments, the Z¹-L¹- group is

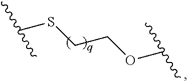

where q is 1-3.

In certain embodiments, the subject compounds comprise a —Z¹-L- group selected from:

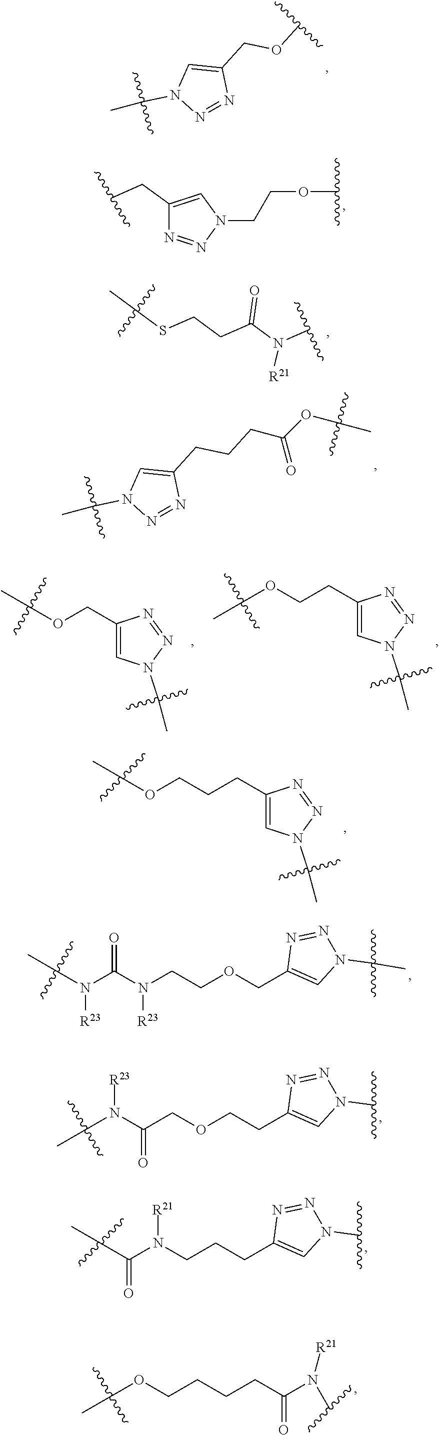

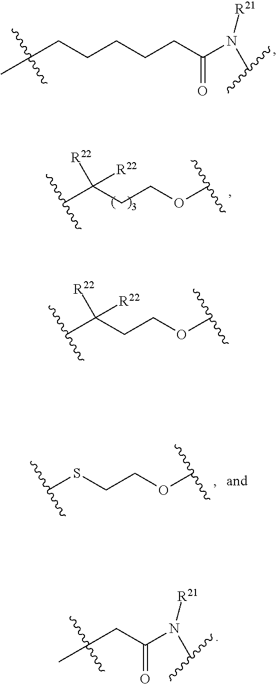

In certain embodiments, the Z¹-L¹- group is

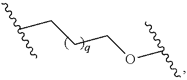

where q is 1-3. In certain embodiments, q is 1. In certain embodiments, q is 2. In certain embodiments, q is 3.

In certain embodiments, the Z¹-L¹- group is

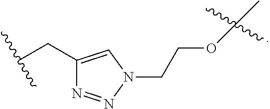 [SMILES: *Cc1cn(CCO*)nn1]

In certain embodiments, the Z¹-L¹- group is

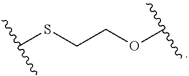 [SMILES: *OCCS*]

In certain embodiments, the Z¹-L¹- group is

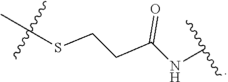 [SMILES: *NC(=O)CCS*]

In certain embodiments, —Z¹-L¹- comprises an optionally substituted —NH-heteroarylene-. In certain embodiments the heteroarylene is a triazole. In certain embodiments, the heteroarylene is pyridine. In certain embodiments, the heteroarylene is pyrimidine. In certain embodiments, the heteroarylene is thiadiazole.

In certain embodiments, the —Z¹-L¹- comprises a group selected from:

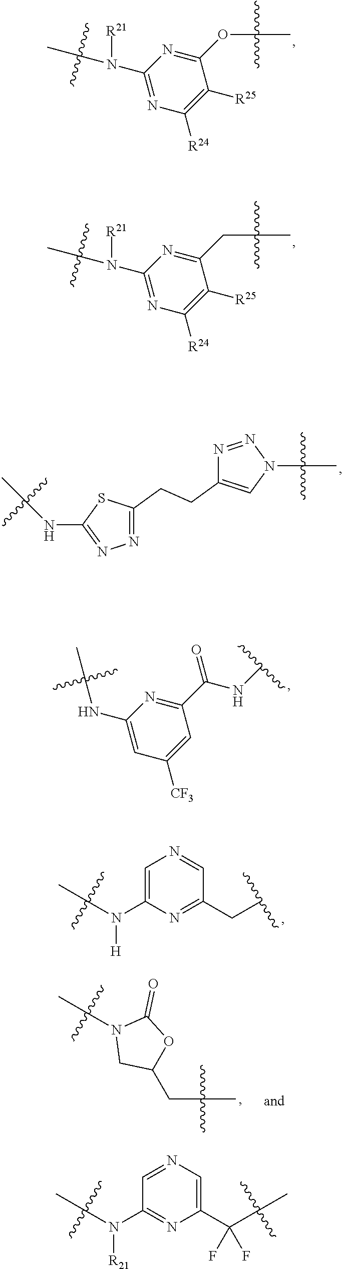

wherein each R²¹ is independently selected from H, optionally substituted (C₁-C₆)alkyl, and optionally substituted acyl; and R²⁴ and R²⁵ are each independently selected from H, optionally substituted C₍₁₋₆₎-alkyl, optionally substituted fluoroalkyl, and halogen.

In certain embodiments, the —Z¹-L¹- comprises a group selected from:

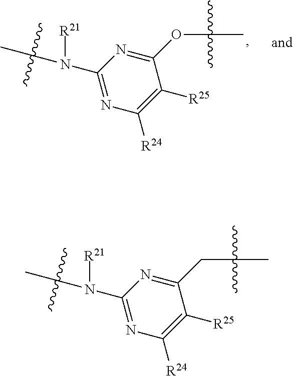

wherein R²⁴ and R²⁵ are each independently selected from H, optionally substituted C₍₁₋₆₎-alkyl, optionally substituted fluoroalkyl, and halogen; and each R²¹ is independently selected from H, optionally substituted (C₁-C₆)alkyl, and optionally substituted acyl.

In certain embodiments, R²¹ is H. In certain embodiments, R²⁴ is C₍₁₋₃₎-alkyl, or C₍₁₋₃₎-fluoroalkyl. In certain embodiments, the fluoroalkyl is CF₃. In certain embodiments, R²⁵ is C₍₁₋₃₎-alkyl, or C₍₁₋₃₎-fluoroalkyl. In certain embodiments, the fluoroalkyl is CF₃.

In certain embodiments, —Z¹-L¹- is

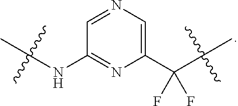 [SMILES: *Nc1cncc(C(*)(F)F)n1]

It is understood that a variety of substituents and chemistries can be utilized to connect a particular X ligand moiety (e.g., as described herein) to an adjacent linker. In some embodiments, a linking moiety of the linker comprises a triazole that derives from a Click chemistry conjugation. In certain embodiments, the ASGPR ligand moiety (X) is attached to a linking moiety as shown in one of the following structures:

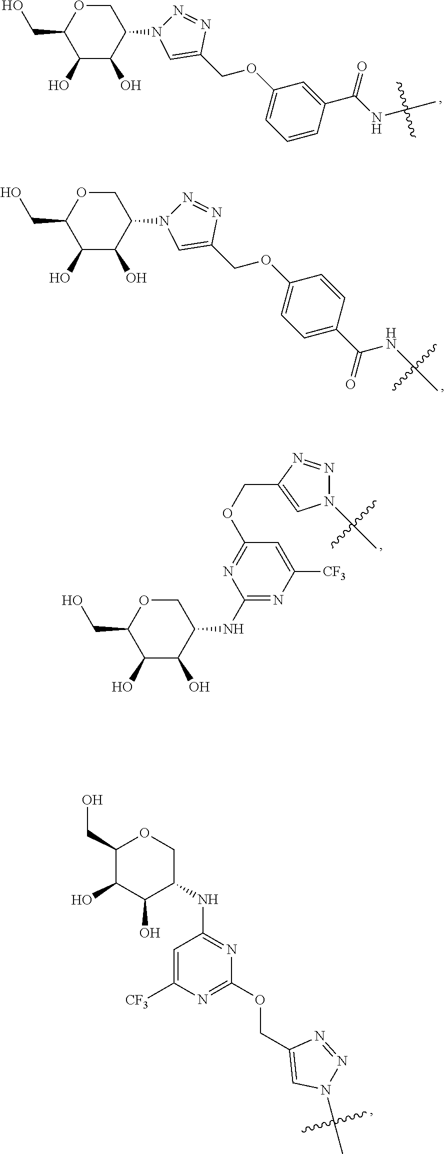 [SMILES: *NC(=O)c1cccc(OCc2cn([C@H]3CO[C@H](CO)[C@H](O)[C@@H]3O)nn2)c1 ; *NC(=O)c1ccc(OCc2cn([C@H]3CO[C@H](CO)[C@H](O)[C@@H]3O)nn2)cc1 ; *n1cc(COc2cc(C(F)(F)F)nc(N[C@H]3CO[C@H](CO)[C@H](O)[C@@H]3O)n2)nn1 ; *n1cc(COc2nc(N[C@H]3CO[C@H](CO)[C@H](O)[C@@H]3O)cc(C(F)(F)F)n2)nn1]

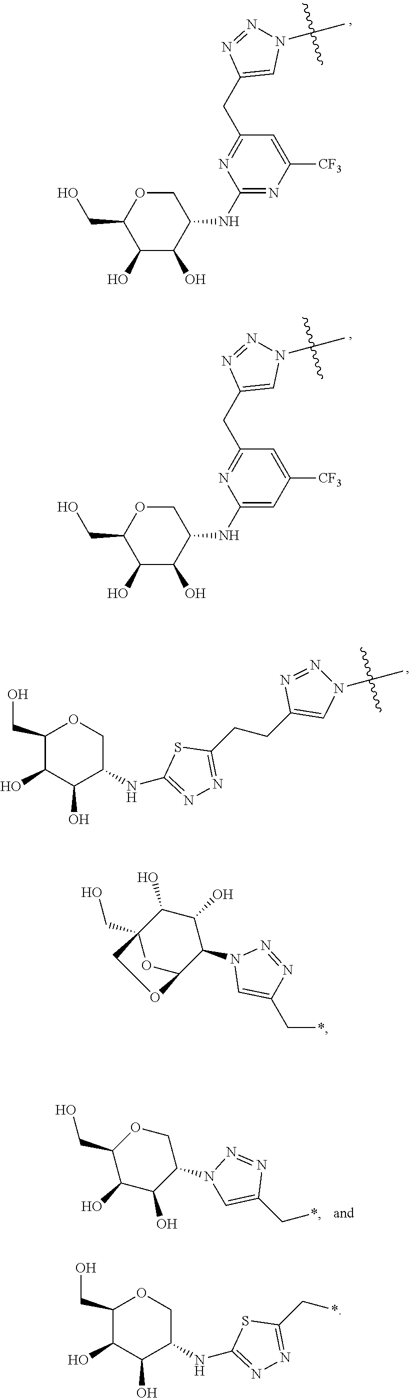

In certain embodiments of formula (Iib), R¹ R³, R⁴, and R¹¹ are H, and R⁶ is OH:

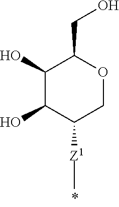

wherein Z¹ is triazole, —NH-heteroaryl (e.g., —NH— attached to pyridine, pyrazine, or pyrimidine), —NH—, —O—, or —CH₂—, and/or Z¹ is attached to a linking moiety as shown in one of the following structures:

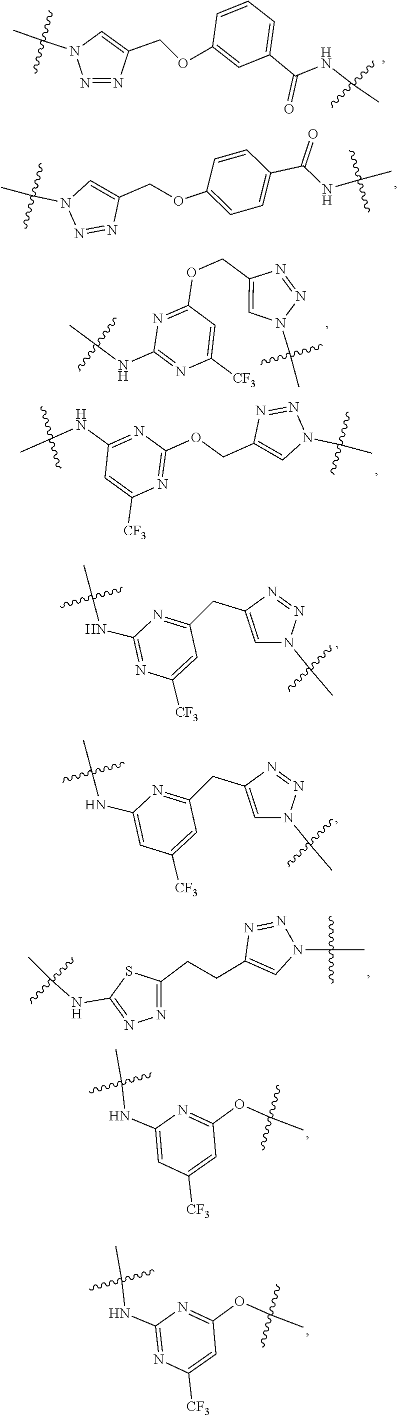

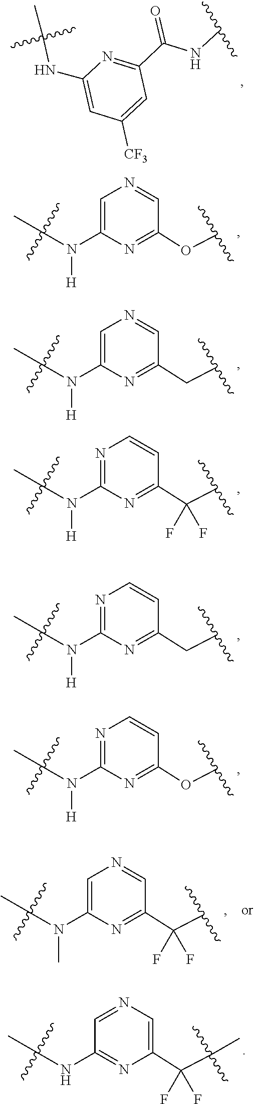

In certain embodiments, of formula (Iib), R¹ R³, R⁴, and R¹¹ are H, and R⁶ is OH:

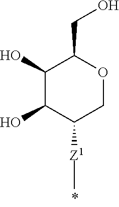

wherein Z¹ is attached to a linking moiety as shown in one of the following structures

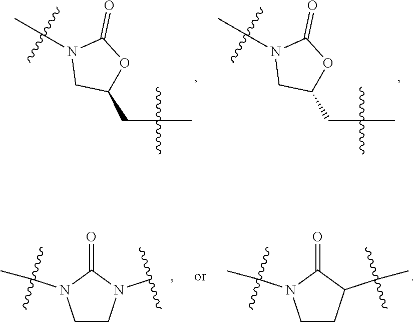 [SMILES: *C[C@H]1CN(*)C(=O)O1 ; *C[C@@H]1CN(*)C(=O)O1 ; *N1CCN(*)C1=O ; *C1CCN(*)C1=O]

### M6PR Ligand Moieties

As summarized above, the M6PR binding moieties (also referred to as M6PR ligand moieties) of this disclosure can be linked to a variety of moieties of interest without impacting the specific binding to, and function of, the cell surface M6PR. The inventors have demonstrated that M6PR binding moieties having particular structures described below provide for high affinity binding to cell surface M6PRs, and when configured via a linker according to the bifunctional compounds of this disclosure can utilize the functions of cell surface M6PRs in a biological system, e.g., for internalization, and/or degradation of a target molecule.

The terms “mannose-6-phosphate receptor” and “M6PR” refer to receptors of the family of mannose-6-phosphate receptors. M6PRs are transmembrane glycoprotein receptors that target enzymes to lysosomes in cells. MP6R endogenously transports proteins bearing N-glycans capped with mannose-6-phosphate (M6P) residues to lysosomes, and cycles between endosomes, the cell surface, and the Golgi complex. See, e.g., Ghosh et al., Nat. Rev. Mol. Cell Biol. 2003; 4: 202-213. The family of M6PRs includes the cation independent mannose-6-phosphate receptor (CI-M6PR). The CI-M6PR is also referred to as the insulin-like growth factor 2 receptor (IGF2R) and is encoded in humans by the IGF2R gene (see, e.g., NCBI Reference Sequence: NM_000876.3, and NCBI Gene ID: 3482). The CI-M6PR binds insulin-like growth factor 2 (IGF-2) and mannose-6-phosphate (M6P)-tagged proteins. The compounds of this disclosure can specifically bind to a cell surface M6PR, for example, an internalizing CI-M6PR cell surface receptor. In particular embodiments, the surface CI-M6PR is a human CI-M6PR. It is understood that the terms M6PR and CI-M6PR are used interchangeably when referring to the binding properties of the M6PR binding moieties and compounds of this disclosure.

A compound comprising such M6PR binding moiety (X) (e.g., as described herein), may bind to other receptors, for example, may bind with lower affinity as determined by, e.g., immunoassays or other assays known in the art. In a specific embodiment, X, or a compound as described herein including such X specifically binds to a cell surface CI-M6PR with an affinity that is at least 2 logs, 2.5 logs, 3 logs, 4 logs or greater than the affinity when X or the compound bind to another cell surface receptor. In a specific embodiment, X, or a compound as described herein comprising X, specifically binds to CI-M6PR with an affinity (K_{d}) 20 mM or less. In particular embodiments, such binding is with an affinity (K_{d}) is 10 mM or less, 1 mM or less, 100 uM or less, 10 uM or less, 1 uM or less, 100 nM or less, 10 nM or less, or 1 nM or less. The terms “binds,” “binds to,” “specifically binds” or “specifically binds to” in this context are used interchangeably.

The M6PR binding compounds of this disclosure include a moiety (X) (e.g., as described herein) which is a D-mannopyranose analog that specifically binds to the cell surface receptor M6PR. The M6PR binding compounds can be monovalent or multivalent (e.g., bivalent or trivalent or of higher valency), where a monovalent compound includes a single M6PR ligand moiety, and a monovalent compound includes two or more such moieties.

### Alpha-Linked Pyranose Ring

The M6PR binding moiety of the compounds of this disclosure can include a linked pyranose ring described by formula (II″):

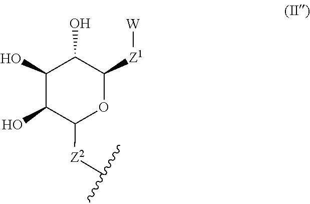

where:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

In some embodiments Z² is not O.

In some embodiments of formula (II″), Z² is a linking moiety connected to the pyranose sugar ring at the anomeric or 1-position with an alpha-configuration as shown in formula (IIa″) below:

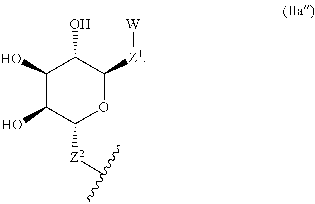

### Beta-Linked Pyranose Ring

The inventors have demonstrated that although M6PR binding compounds having a M6PR binding moiety with an anomeric alpha-configuration of formula (IIa″) can provide good binding and internalization activity at the receptor, in some cases it is possible to impart more potent binding and internalization activity at the M6PR by configuring the central pyranose sugar ring of the M6PR binding moiety with a beta-configuration at the anomeric position. In some embodiments, such M6PR binding moieties can provide for increased stability at the pyranose ring.

Accordingly, in some embodiments of formula (II″), Z² is a linking moiety connected to the sugar ring at the anomeric or 1-position with a beta-configuration as shown in formula (IIb″) below:

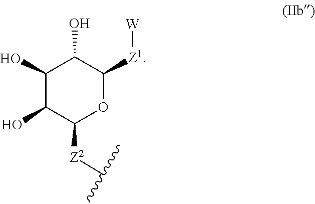

### Additional M6PR Ligand Moieties

Although moieties of formula (II″) can exhibit binding activity for the M6PR, the inventors have demonstrated that when particular types of cyclic groups are linked with a particular configuration adjacent to the pyranose ring of formula (II″) via the linking moiety Z², a M6PR binding moiety of desirable binding activity can be produced.

Accordingly, in some embodiments of formula (II″), the M6PR binding moiety (X) can be described by formula (III″):

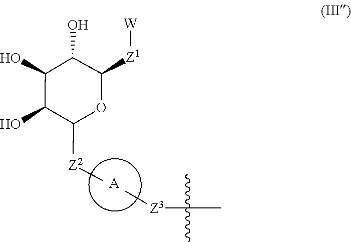

or a prodrug thereof, or a salt thereof, wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
A is independently an optionally substituted cyclic group; and
Z³ is independently a linking moiety.

In some embodiments of formula (II″)-(III″), W is a non-hydrolyzable hydrophilic head group.

In some embodiments of formula (Z² is optionally substituted ethylene. In some embodiments of formula Z² is optionally substituted ethenylene.

In some embodiments of formula (II″)-(III″), Z² is O. In some embodiments of formula (II″)-(III″), Z² is S. In some embodiments of formula (II″)-(III″), Z² is —NR²¹—. In some embodiments of formula (II″)-(III″), Z² is —C(R²²)₂—, wherein each R²² is independently selected from H, halogen (e.g., F) and optionally substituted (C₁-C₆)alkyl. In some embodiments of formula (II″)-(III″), Z² is —CH₂—.

In some embodiments of formula (II″)-(III″), A is optionally substituted aryl, optionally substituted heteroaryl, optionally substituted heterocycle, or optionally substituted cycloalkyl. In some embodiments of formula (II″)-(III″), A is independently an optionally substituted aryl or heteroaryl linking moiety (e.g., monocyclic or bicyclic aryl or heteroaryl, optionally substituted).

Exemplary Z³ linking moieties of formula (II″)-(III″) are described herein.

Such M6PR-binding moieties of formula (III″) can be attached to a moiety or molecule of interest to produce a bifunctional compound that undergoes effective M6PR-mediated cell internalization. The inventors have further demonstrated that when the moiety or molecule of interest is a target protein-binding moiety, the M6PR binding compound also provides for M6PR mediated internalization and/or degradation of bound target protein.

Accordingly, the M6PR binding compound is of formula (XII):

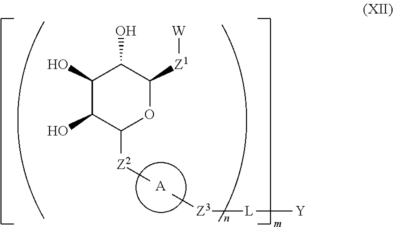

or a prodrug thereof, or a salt thereof,
wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
A is independently an optionally substituted cyclic group;
Z³ is independently a linking moiety;
n is 1 to 500;
L is a linker;
Y is a moiety of interest; and
m is 1 to 100.

In some embodiments the cell surface M6PR binding compound is of formula (XIII):

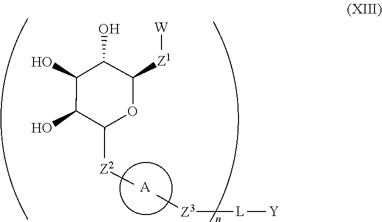

or a prodrug thereof, or a salt thereof,
wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
A is independently an optionally substituted cyclic group;
Z³ is independently a linking moiety;
n is 1 to 500;
L is a linker; and
Y is a moiety of interest (e.g., as described herein).

In some embodiments of formula (XIII), Y is a chemoselective ligation group. In some embodiments of formula (XIII), n is 1. In some embodiments of formula (XIII), Y is a chemoselective ligation group connected to “n” M6PR binding moieties (Xn-) via a single linker -L-. In some embodiments of formula (XIII), n is 2, 3, 4, or 5. In some embodiments of formula (XIII), n is 5-10. In some embodiments of formula (XIII), n is 10-100, such as 20-80, or 20-50. In some embodiments of formula (XIII), when n is 5 or more, then L is a polypeptide containing linker (e.g., as described herein).

In some embodiments of formula (XII)-(XIII), when n is 1 and A is phenyl, then: i) L comprises a backbone of at least 16 consecutive atoms (e.g., at least 18 consecutive atoms, or at least 20 consecutive atoms, in some cases up to about 200 consecutive atoms); ii) Y is a biomolecule; and/or ii) Z³ is amide, sulfonamide, urea or thiourea linking moiety to linker L.

In some embodiments of formula (XII), Z² is a linking moiety connected to the sugar ring at the anomeric or 1-position with an alpha-configuration as shown in formula (IIa) such that the compound is of formula (XIIa):

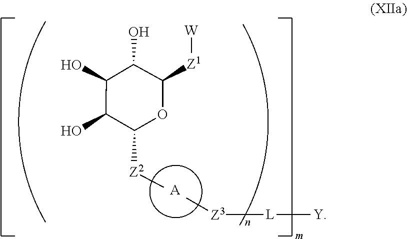

In some embodiments of formula (XII), Z² is a linking moiety connected to the sugar ring at the anomeric or 1-position with a beta-configuration as shown in formula (IIb), such that the compound is of formula (XIIb):

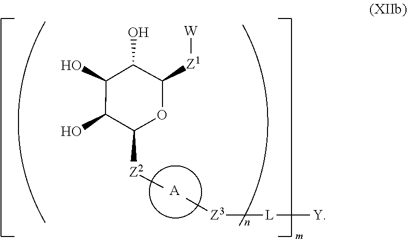

In some embodiments of formula (XI)-(XIIb), multiple M6PR binding moieties, e.g., of formula (III), are linked via multiple linkers L to different ligation sites on a moiety of interest Y. In some embodiments, when Y is a biomolecule, the compound of formula (XI)-(XIIb) can be referred to as a conjugate.

### Hydrophilic Head Groups and Linking Moieties

In some embodiments of formula (II)-(XIII), the M6PR binding moiety (X) includes an analog of a D-mannopyranose ring, with a hydrophilic head group, or a precursor or prodrug thereof, that is connected via a linking moiety (Z¹) to the 5-position of the sugar ring. The linking moiety can be of 1-6 atoms in length, such as 1-5, 1-4 or 1-3 atoms in length, e.g., 1 or 2 atoms in length. It is understood that the length of the linking moiety can be selected in conjunction with the hydrophilic head group.

The hydrophilic head group (W) can be any suitable negatively charged group, or salt thereof. In some embodiments, the hydrophilic head group is a neutral, polar, hydrophilic group. In general, the hydrophilic head group is capable of hydrogen bonding or electrostatic interactions with the M6PR, under aqueous or physiological conditions, similar to those of the phosphate group of M6P. The hydrophilic head group can be a bioisostere (e.g., a structural or functional mimic) of the 6-phosphate group of the naturally occurring mannose-6-phosphate ligand. In some embodiments, the hydrophilic head group is non-hydrolyzable, i.e., a functional group that is stable against its cleavage (e.g., chemically or enzymatically) under physiological conditions, from the Z¹ linking moiety and/or pyranose ring of X to which the hydrophilic head group is attached.

The hydrophilic head group is generally a small group, such as a heteroatom containing functional group, or single heterocyclic ring, and in some cases has a MW of less than 200, such as less than 150, or less than 100.

In some embodiments, the hydrophilic head group is a phosphonate, or a bioisostere thereof, such as a carboxylate or malonate. In some embodiments, the hydrophilic head group is a thiophosphonate.

In some embodiments of formula (II)-(XIII), the hydrophilic head group is not a phosphate, thiophosphate or dithiophosphate, as such groups would have phosphate ester linkages to the compound which can be unstable and susceptible to cleavage under physiological conditions (e.g., by phosphatases in a biological system or chemically). For example, the 6-phosphate ester group of M6P exhibits undesirable stability as compared to a phosphonate analog, or other more stable head group. This disclosure provides alternative non-hydrolyzable head groups in addition to phosphonate which retain binding and internalization activity of the resulting M6PR binding compound.

In any one of the embodiments of formula (II)-(XIII), the hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

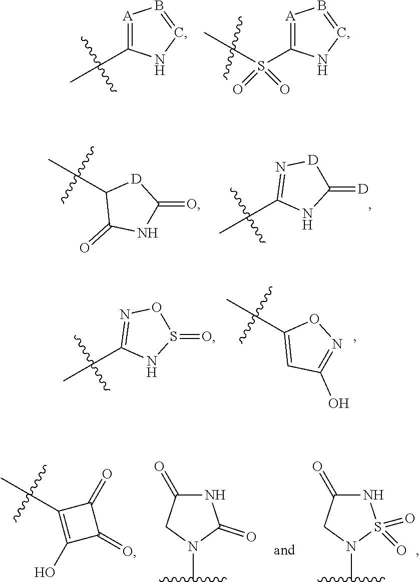

or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

In some embodiments of formula (II)-(XIII), the hydrophilic head group W is phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof. In some embodiments of formula (II)-(XIII), the hydrophilic head group W is non-hydrolyzable, and accordingly, is not selected from phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof.

In some embodiments of formula (II)-(XIII), the hydrophilic head group W is charged, e.g., capable of forming a salt under aqueous or physiological conditions. In some embodiments of formula (II)-(XIII), the hydrophilic head group W is selected from —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —COOH, —CH(COOH)₂, —CR¹R²COOH, and —NHC(O)CO₂H.

In some embodiments of formula (II)-(XIII), the hydrophilic head group W is phosphonate or thiophosphonate (e.g., —P═O(OH)₂, —P═S(OH)₂, —P═O(SH)(OH), or —P═S(SH)(OH), or a salt thereof). In some embodiments of formula (II)-(XIII), the hydrophilic head group W is phosphonate or a salt thereof. In some embodiments of formula (II)-(XIII), the hydrophilic head group W is —CO₂H or a salt thereof. In some embodiments of formula (II)-(XIII), the hydrophilic head group W is malonate (e.g., —CH(COOH)₂ or a salt thereof).

In some embodiments of formula (II)-(XIII), the hydrophilic head group W is selected from —SO₂OH (i.e., —SO₃H), —S(O)OH, —OSO₂OH, and —NHSO₃H. In some embodiments of formula (II)-(XIII), the hydrophilic head group W is sulfonate (e.g., —SO₃H or a salt thereof).

In some embodiments, the hydrophilic head group W is neutral hydrophilic. In some embodiments of formula (II)-(XIII), the hydrophilic head group W is selected from —OH, —CR²R²OH, —CN, —CONH₂, —CONHR³, —CONR³R⁴, —CONH(OH), —CONH(OR³), —CONHSO₂R³, —SO₂R³, —SOR³R⁴, —SO₂NH₂, —SO₂NHR³, —SO₂NR³R⁴, —SO₂NHCOR³, —NHCOR³, —NHSO₂NHR³, —NHC(O)NHS(O)₂R³, and —NHSO₂R³.

In some embodiments of formula (II)-(XIII), the hydrophilic head group W comprises a heterocycle, such as

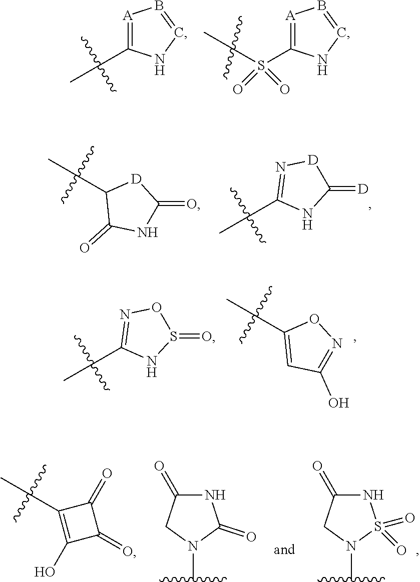

and or a salt thereof,
wherein A, B, and C are each independently CH or N; and D is each independently O or S.

In some embodiments of formula (II)-(XIII), the hydrophilic head group W comprises a 5-membered heterocycle, such as

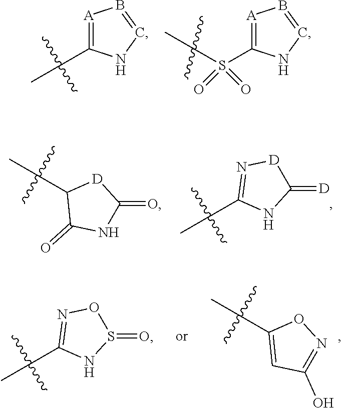

or H or a salt thereof.

In some embodiments of formula (II)-(XIII), the hydrophilic head group W is linked to the pyranose ring via a Z¹ that is selected from optionally substituted (C₁-C₂)alkylene and optionally substituted ethenylene. The Z¹ can be selected in conjunction with W so as to provide a desired spacing between the 5-position of the ring and the charged or polar center of W. For example, when W is a malonate having a CH atom linking the two carboxylic acid groups, Z¹ can be methylene, which together provide a desirable two carbon spacer between the ring and the COOH groups.

In some embodiments of formula (II)-(XIII), Z¹ is methylene or substituted methylene. In some embodiments of formula (II)-(XIII), Z¹ is ethyl or substituted ethyl. In some embodiments of formula (II)-(XIII), Z¹ is ethenylene or substituted ethenylene. In some embodiments of formula (II)-(XIII), Z¹ is substituted with one or more halogen, e.g., fluoro.

In some embodiments of formula (III), the M6PR binding moiety (X) is described by one of formula (IV-1) to (IV-3):

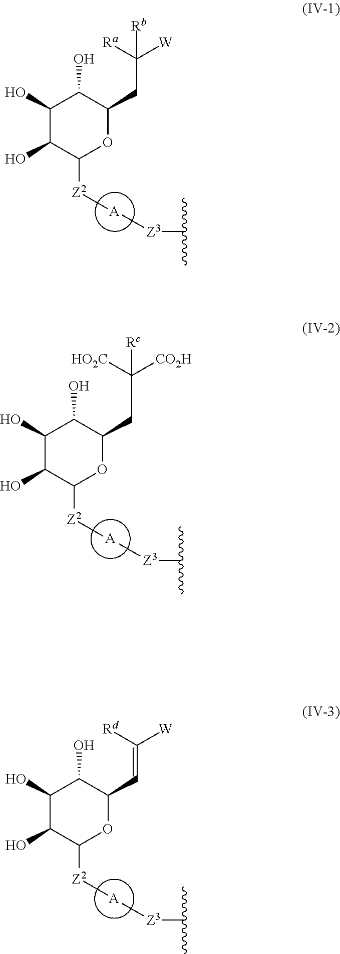

wherein R^{a}, Rb, R^{c} and R^{d} are independently H or F.

In some embodiments of formula (IV-1) to (IV-3), Z² is O.

In some embodiments of formula (IV-1) to (IV-3), Z² is S.

In some embodiments of formula (IV)-1 to (IV-3), Z² is —NR²¹—.

In some embodiments of formula (IV-1) to (IV-3), Z² is —C(R²²)₂—, wherein each R²² is independently selected from H, halogen (e.g., F) and optionally substituted (C₁-C₆)alkyl. In some embodiments of formula (IV-1) to (IV-3), Z² is —CH₂—.

In some embodiments of formula (IV-1) to (IV-3), R^{a}, R^{b}, R^{c} and R^{d} are each H.

In some embodiments of formula (IV-1) R^{a} is H and R^{b} is F. In some embodiments of formula (IV-1) R^{a} and R^{b} are each F.

In some embodiments of formula (IV-2) R^{c} is H. In some embodiments of formula (IV-2) R^{c} is F.

In some embodiments of formula (IV-3) R^{d} is H. In some embodiments of formula (IV-3) R^{d} is F.

In some embodiments of formula (IV-1) and (IV-3), W is selected from —P═O(OH)₂, —P═S(OH)₂, —P═O(SH)(OH), —P═S(SH)(OH), and —COOH, or a salt thereof. In some embodiments of formula (IV-1) and (IV-3), W is —P═O(OH)₂, or a salt thereof. In some embodiments of formula (IV-1) and (IV-3), W is COOH, or a salt thereof.

In some embodiments of formula (IV-1) R^{a} and R^{b} are each F, and W is —P═O(OH)₂, or a salt thereof. In some embodiments of formula (IV-1) R^{a} and R^{b} are each H, and W is —P═O(OH)₂, or a salt thereof. In some embodiments of formula (IV-1) R^{a} is F, R^{b} is H, and W is —P═O(OH)₂, or a salt thereof.

In some embodiments of formula (IV-1) to (IV-3), Z² is linked to the anomeric position of the pyranose ring with an alpha-configuration. In such cases, the M6PR binding moiety (X) of (IV-1) to (IV-3) can be referred to as formula (IV-A1) to (IV-A3), respectively.

In some embodiments of formula (IV-A1) to (IV-A3), Z² is S. In some embodiments of formula (IV-A1) to (IV-A3), Z² is O. In some embodiments of formula (IV-A1) to (IV-A3), Z² is —CH₂—. In some embodiments of formula (IV-A1) to (IV-A3), Z² is —CF₂—.

In some embodiments of formula (IV-A1) and (IV-A3), W is selected from —P═O(OH)₂, —P═S(OH)₂, —P═O(SH)(OH), —P═S(SH)(OH), and —COOH, or a salt thereof. In some embodiments of formula (IV-A1) and (IV-A3), W is —P═O(OH)₂, or a salt thereof. In some embodiments of formula (IV-A1) and (IV-A3), W is COOH, or a salt thereof.

In some embodiments of formula (IV-A1) R^{a} and R^{b} are each F, and W is —P═O(OH)₂, or a salt thereof. In some embodiments of formula (IV-A1) R^{a} and R^{b} are each H, and W is —P═O(OH)₂, or a salt thereof. In some embodiments of formula (IV-A1) R^{a} is F, R^{b} is H, and W is —P═O(OH)₂, or a salt thereof.

In some embodiments of formula (IV-1) to (IV-3), Z² is linked to the anomeric position of the pyranose ring with a beta-configuration. The inventors demonstrated that a compound including a M6PR binding moiety having a β-glycoside configuration can have at least equivalent binding and/or cellular uptake activity as compared to a conjugate having the corresponding α-glycoside configuration. In some embodiments, such M6PR binding moieties having a β-glycoside configuration can provide increased stability as compared to a reference compound having a β-glycoside configuration. Accordingly, in some embodiments of formula (IV), the M6PR binding moiety (X) is described by one of formula (IV-B1) to (IV-B3):

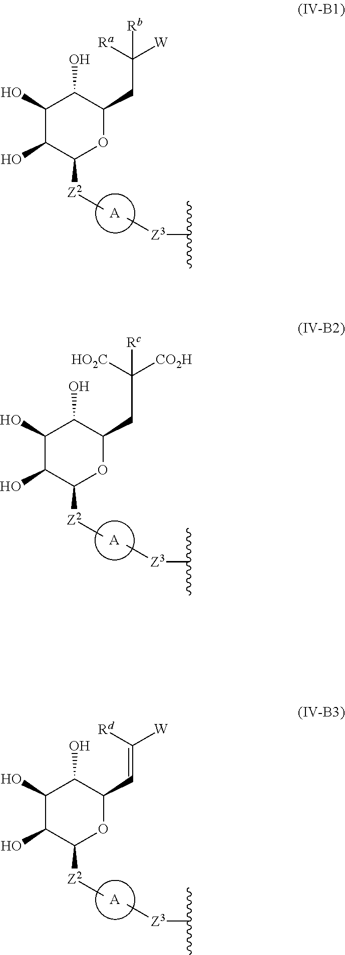

wherein R^{a}, R^{b}, R^{c} and R^{d} are independently H or F.

In some embodiments of formula (IV-B1) to (IV-B3), Z² is S. In some embodiments of formula (IV-B1) to (IV-B3), Z² is 0. In some embodiments of formula (IV-B1) to (IV-B3), Z² is —CH₂—. In some embodiments of formula (IV-B1) to (IV-B3), Z² is —CF₂—.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof. In some embodiments of formula (IV-B1) and (IV-B3), W is —P═O(OH)₂, or a salt thereof. In some embodiments of formula (IV-B1) and (IV-B3), W is COOH, or a salt thereof.

In some embodiments of formula (IV-B1) R^{a} and R^{b} are each F, and W is —P═O(OH)₂, or a salt thereof. In some embodiments of formula (IV-B1) R^{a} and R^{b} are each H, and W is —P═O(OH)₂, or a salt thereof. In some embodiments of formula (IV-B1) R^{a} is F, R^{b} is H, and W is —P═O(OH)₂, or a salt thereof.

The inventors demonstrated that a conjugate including M6PR binding moiety having a β-S-glycoside configuration can have at least equivalent or superior binding and/or cellular uptake activity as compared to a conjugate having the corresponding α-S-glycoside configuration, or to a conjugate having an α-O-glycoside configuration.

Accordingly, in some embodiments of formula (IV-B1) to (IV-B3), the M6PR binding moiety (X) is described by one of formula (IV-BS1) to (IV-BS3):

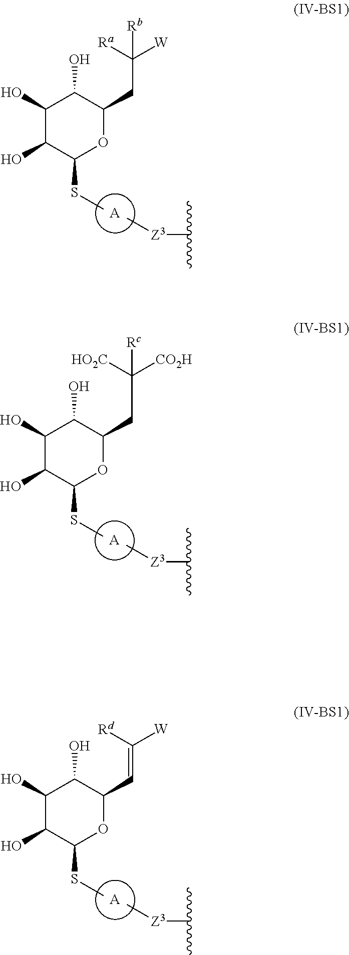

wherein R^{a}, R^{b}, R^{c} and R^{d} are independently H or F.

In some embodiments of formula (IV-BS1) to (IV-BS3), R^{a}, R^{b}, R^{c} and R^{d} are each H.

In some embodiments of formula (IV-BS1) R^{a} is H and R^{b} is F. In some embodiments of formula (IV-BS1) R^{a} and Rb are each F.

In some embodiments of formula (IV-BS2) R^{c} is H. In some embodiments of formula (IV-B2) R^{c} is F.

In some embodiments of formula (IV-BS3) R^{d} is H. In some embodiments of formula (IV-BS3) R^{d} is F.

In some embodiments of formula (IV-BS1) to (IV-BS3), Z² is S. In some embodiments of formula (IV-BS1) to (IV-BS3), Z² is 0. In some embodiments of formula (IV-BS1) to (IV-BS3), Z² is —CH₂—. In some embodiments of formula (IV-BS1) to (IV-BS3), Z² is —CF₂—.

In some embodiments of formula (IV-BS1) and (IV-BS3), W is selected from —P═O(OH)₂, —P═S(OH)₂, —P═O(SH)(OH), —P═S(SH)(OH), and —COOH, or a salt thereof. In some embodiments of formula (IV-BS1) and (IV-BS3), W is —P═O(OH)₂, or a salt thereof. In some embodiments of formula (IV-BS1) and (IV-BS3), W is COOH, or a salt thereof.

In some embodiments of formula (IV-BS1) R^{a} and R^{b} are each F, and W is —P═O(OH)₂, or a salt thereof. In some embodiments of formula (IV-BS1) R^{a} and R^{b} are each H, and W is —P═O(OH)₂, or a salt thereof. In some embodiments of formula (IV-BS1) R^{a} is F, R^{b} is H, and W is —P═O(OH)₂, or a salt thereof.

In some embodiments, the mannose ring or analog thereof of the M6PR binding moiety can be incorporated into the compounds of this disclosure by attachment of a linking moiety to the Z² group attached at the anomeric or 1-position of the sugar ring.

In some embodiments, the M6PR binding moiety is incorporated into the compounds of this disclosure by attachment of a linker to the Z³ group attached to the cyclic group A. It is understood that in the compounds of formula (III), the cyclic group attached to Z² can be considered part of the M6PR binding moiety (X) and provide for a desirable binding property to the M6PR.

### Cyclic Group A

The A cyclic group of formula (III)-(XIII) can be a monocyclic or bicyclic group. A bicyclic group of interest can be a fused bicyclic group or a bicyclic group containing two monocyclic linked via a covalent bond. The A cyclic group of formula (III)-(XIII) can be optionally substituted aryl, optionally substituted heteroaryl, optionally substituted heterocycle (e.g., saturated heterocycle), or optionally substituted cycloalkyl.

The A cyclic group of formula (III)-(XIII) can be a monocyclic aryl or monocyclic heteroaryl group. In some embodiments of formula (III)-(XIII), A is a 5-membered monocyclic heteroaryl group. In some embodiments of formula (III)-(XIII), A is a 6-membered monocyclic aryl or heteroaryl group. In some embodiments of formula (III)-(XIII), A can be a multicyclic aryl or multicyclic heteroaryl group, such as a bicyclic aryl or bicyclic heteroaryl group. In some embodiments of formula (III)-(XIII), A is a fused bicyclic group. In some embodiments of formula (III)-(XIII), A is a bicyclic group comprising two aryl and/or heteroaryl monocyclic rings connected via a covalent bond. In some embodiments of formula (III)-(XIII), A is a bicyclic aryl or bicyclic heteroaryl group having two 6-membered rings. In some embodiments of formula (III)-(XIII), A is a bicyclic aryl or bicyclic heteroaryl group having one 6-membered ring that is connected via a covalent bond or fused to a 5-membered ring.

In some embodiments of formula (III)-(XIII), A is selected from optionally substituted phenyl, optionally substituted pyridyl, optionally substituted biphenyl, optionally substituted naphthalene, optionally substituted quinoline, optionally substituted triazole and optionally substituted phenylene-triazole.

In some embodiments of formula (III)-(XIII), A is not phenyl (also referred to as phenylene in the context of formula (III), e.g., 1,4-phenylene).

In some embodiments of formula (III)-(XIII), A is substituted with at least one OH substituent. In some embodiments of formula (III)-(XIII), A is substituted with 1, 2, or more OH groups. In some embodiments of formula (III)-(XIII), A is substituted with at least one optionally substituted (C₁-C₆)alkyl.

In some embodiments of formula (III)-(XIII), A is optionally substituted 1,4-phenylene, optionally substituted 1,3-phenylene, or optionally substituted 2,5-pyridylene.

In some embodiments of formula (III)-(XIII), A is selected from:

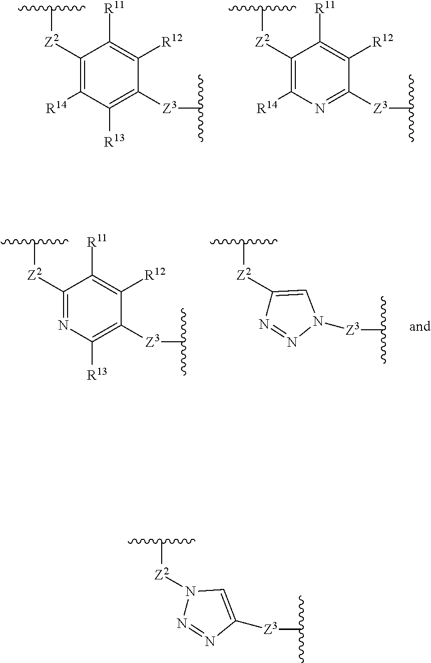

wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
R²⁵ is independently selected from H, and optionally substituted (C₁-C₆)alkyl.

In some embodiments of formula (III)-(XIII), A is optionally substituted fused bicyclic aryl or optionally substituted fused bicyclic heteroaryl.

In some embodiments of formula (III)-(XIII), A is optionally substituted naphthalene or optionally substituted quinoline.

In some embodiments of formula (III)-(XIII), A is selected from:

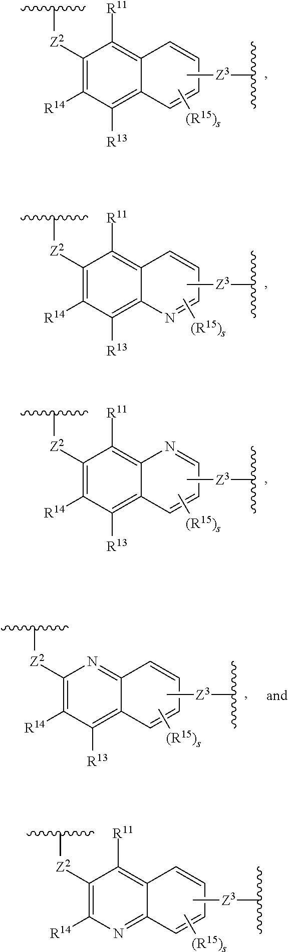

wherein:
R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

In some embodiments of formula (III)-(XIII), A is selected from:

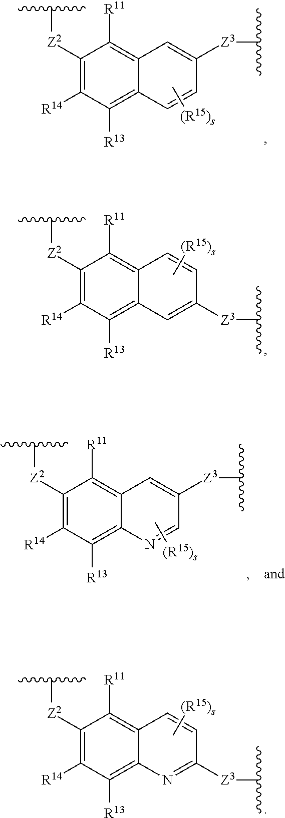

In some embodiments of formula (III)-(XIII), A is optionally substituted bicyclic aryl or optionally substituted bicylic heteroaryl of following formula:

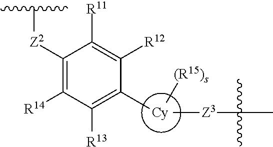

or a salt thereof, wherein:
Cy is independently monocyclic aryl or monocyclic heteroaryl; R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²⁵ is independently selected from H, and optionally substituted (C₁-C₆)alkyl.

In some embodiments, when Cy is optionally substituted phenyl, then A is optionally substituted biphenyl of the formula:

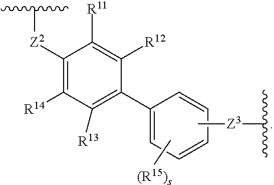

In some embodiments of formula (III)-(XIII), A is selected from:

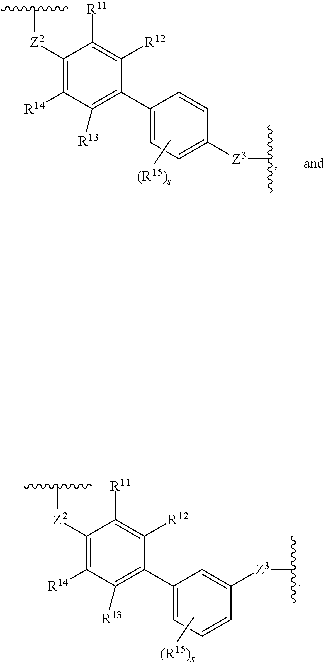

In some embodiments, when Cy is triazole, then A is selected from:

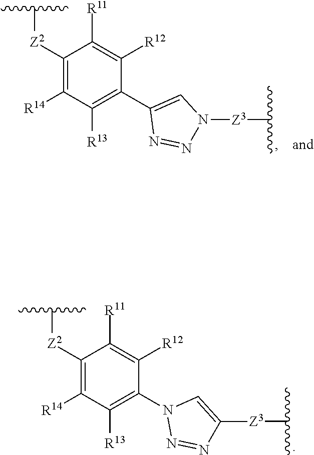

In some embodiments, at least one of R¹¹ to R¹⁵ is OH (e.g., at least two are OH).

In some embodiments, R¹¹ to R¹⁵ are each H.

### Linking Moiety Z³

The linking moiety Z³ can be any convenient linking moiety that connects the linker L to the cyclic ring A. In some embodiments of formula (III)-(XIII), Z³ is has a backbone of 3 atoms or less.

In some embodiments of formula (III)-(XIII), Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (III)-(XIII), Z³ is a covalent bond connecting A to L.

In some embodiments of formula (III)-(XIII), Z³ is optionally substituted amido, urea or thiourea.

In some embodiments of formula (III)-(XIII), Z³ is

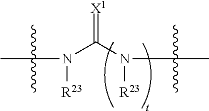

wherein:
X¹ is O or S;
t is 0 or 1; and
each R²³ is independently selected from H, C₍₁₋₃₎-alkyl (e.g., methyl or ethyl) and substituted C₍₁₋₃₎-alkyl. In some embodiments of Z³, X is O. In some embodiments of Z³, X¹ is S. In some embodiments of Z³, t is 0 and X is O, such that Z³ is amido. In some embodiments of Z³, t is 1 such that Z³ is urea or thiourea.

In some embodiments of formula (III)-(XIII), Z³ is —N(R²³)SO₂— or —SO₂N(R²³)—. In some embodiments of formula (III)-(XIII), Z³ is —NHSO₂— or —SO₂NH—.

In some embodiments of formula (III)-(XIII), Z³ is —N(R²³)CO— or —CON(R²³)—. In some embodiments of formula (III)-(XIII), Z³ is —NHCO— or —CONH—.

In some embodiments of formula (III)-(XIII), Z³ is —NHC(═X¹)NH—, wherein X¹ is O or S. In some embodiments, X¹ is O (i.e., Z³ is —NHC(═O)NH—). In some embodiments, X¹ is S.

In some embodiments of formula (III)-(XIII), Z³ is optionally substituted triazole. When Z³ is optionally substituted triazole, it can be synthetically derived from click chemistry conjugation of an azido containing precursor and an alkyne containing precursor of the compound.

In some embodiments, Z³ is selected in combination with cyclic group A and/or linking moiety Z¹ to provide desirable M6PR binding and internalization properties for X.

In some embodiments of formula (III)-(XIII), -A-Z³— is selected from:

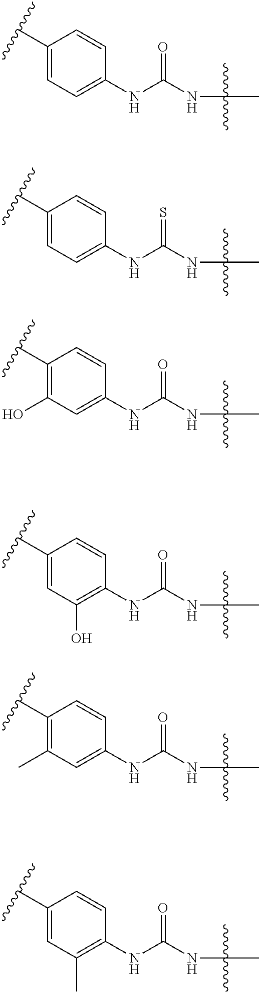 [SMILES: *NC(=O)Nc1ccc(*)cc1 ; *NC(=S)Nc1ccc(*)cc1 ; *NC(=O)Nc1ccc(*)c(O)c1 ; *NC(=O)Nc1ccc(*)cc1O ; *NC(=O)Nc1ccc(*)c(C)c1 ; *NC(=O)Nc1ccc(*)cc1C]

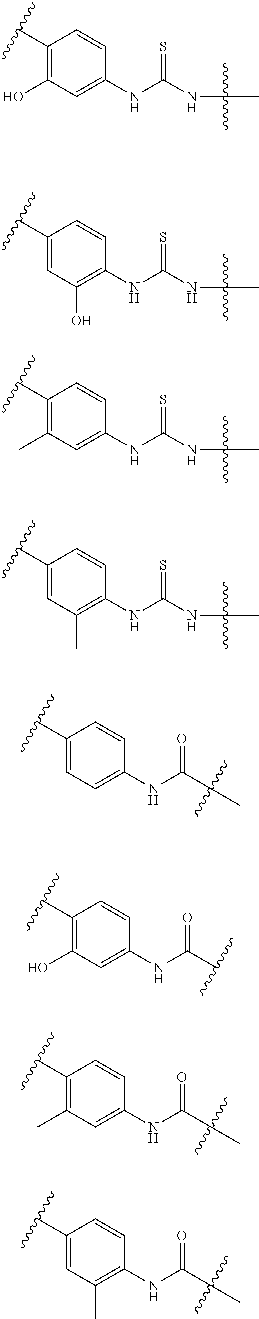

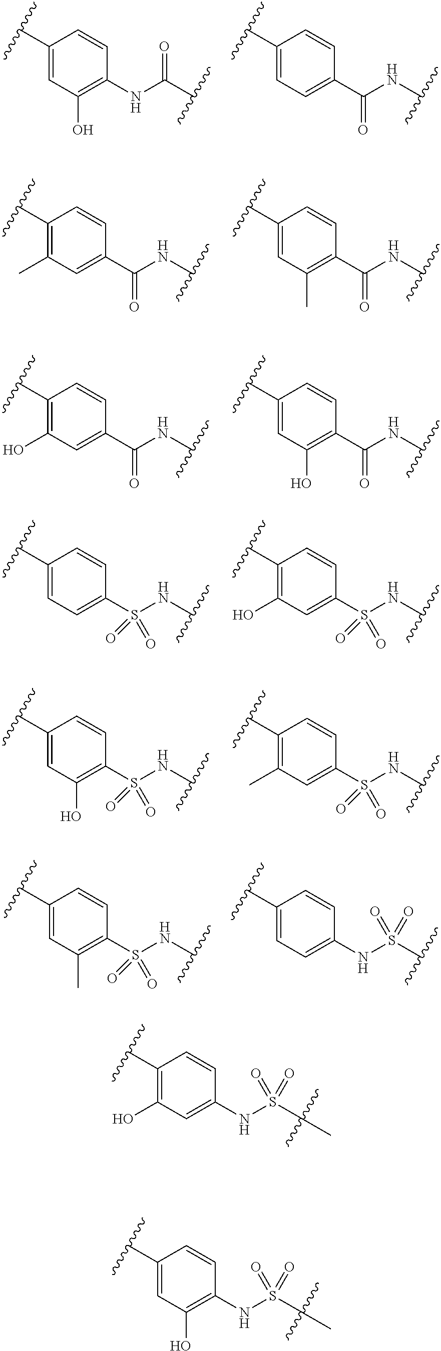

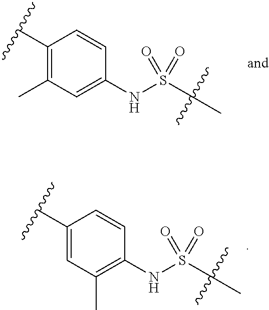 [SMILES: *c1ccc(NS(*)(=O)=O)cc1C ; *c1ccc(NS(*)(=O)=O)c(C)c1]

In some embodiments of formula (III)-(XIII), -A-Z³— is selected from:

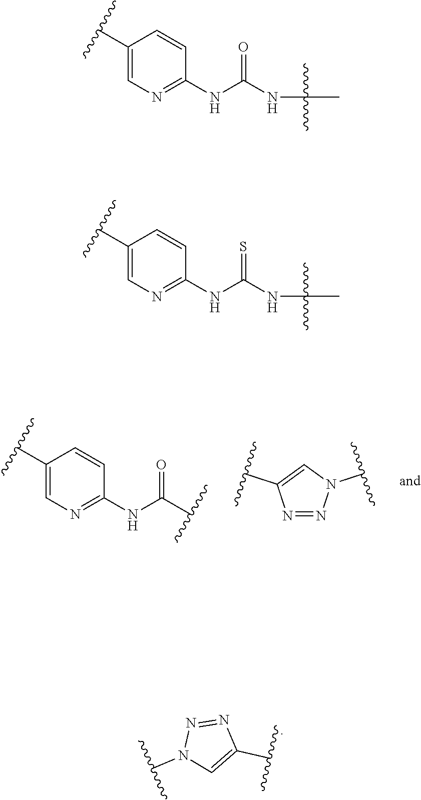 [SMILES: *NC(=O)Nc1ccc(*)cn1 ; *NC(=S)Nc1ccc(*)cn1 ; *C(=O)Nc1ccc(*)cn1 ; *c1cn(*)nn1 ; *c1cn(*)nn1]

In some embodiments of formula (III)-(XIII), -A-Z³— is selected from:

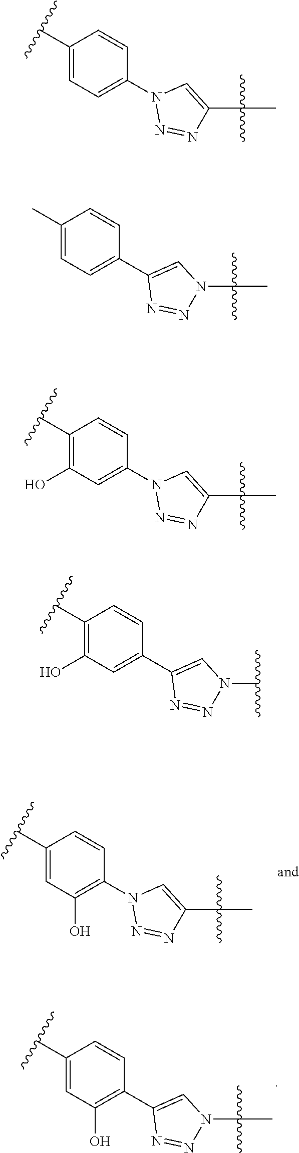 [SMILES: *c1ccc(-n2cc(*)nn2)cc1 ; *n1cc(-c2ccc(C)cc2)nn1 ; *c1cn(-c2ccc(*)c(O)c2)nn1 ; *c1ccc(-c2cn(*)nn2)cc1O ; *c1ccc(-n2cc(*)nn2)c(O)c1 ; *c1ccc(-c2cn(*)nn2)c(O)c1]

In some embodiments of formula (II)-(XIb), -A-Z³— is selected from:

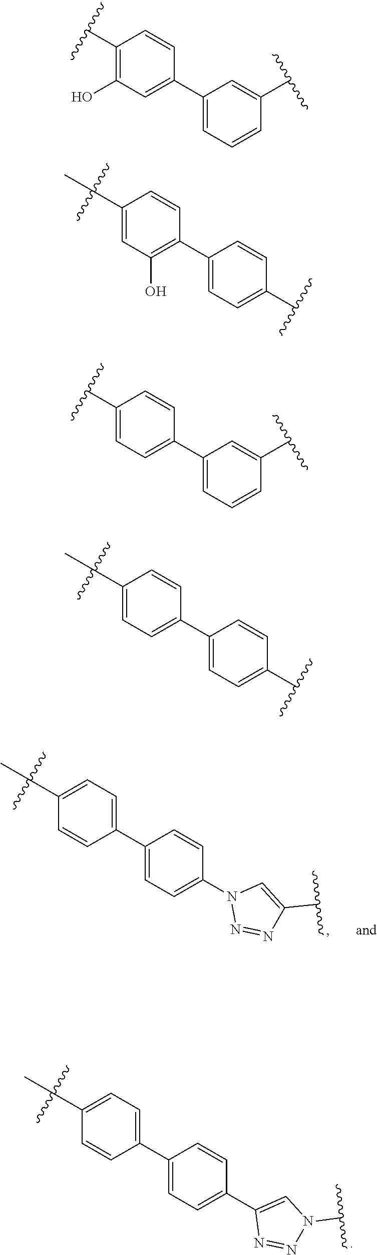 [SMILES: *c1cccc(-c2ccc(*)c(O)c2)c1 ; *c1ccc(-c2ccc(*)cc2O)cc1 ; *c1ccc(-c2cccc(*)c2)cc1 ; *c1ccc(-c2ccc(*)cc2)cc1 ; *c1ccc(-c2ccc(-n3cc(*)nn3)cc2)cc1 ; *c1ccc(-c2ccc(-c3cn(*)nn3)cc2)cc1]

In some embodiments of formula (III)-(XIII), -A-Z³— is selected from:

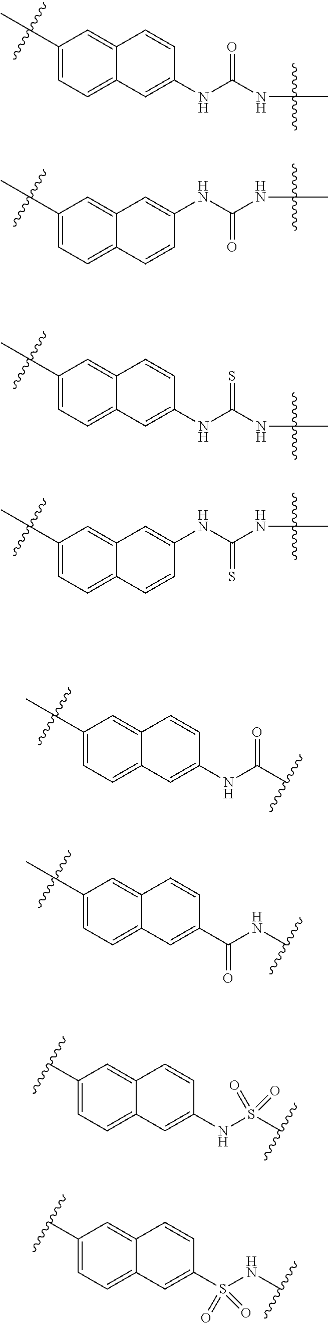

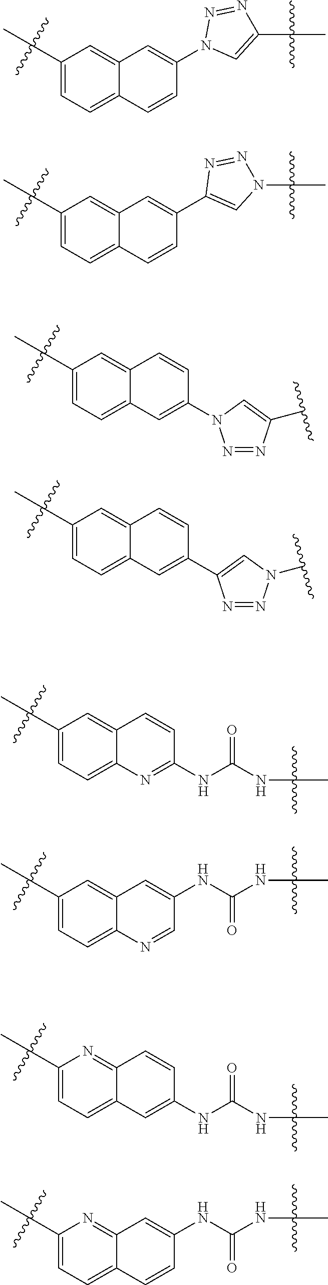

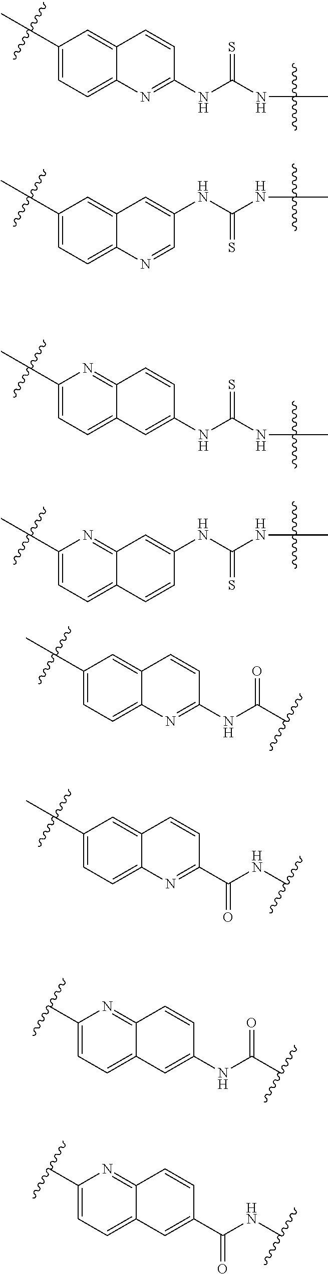

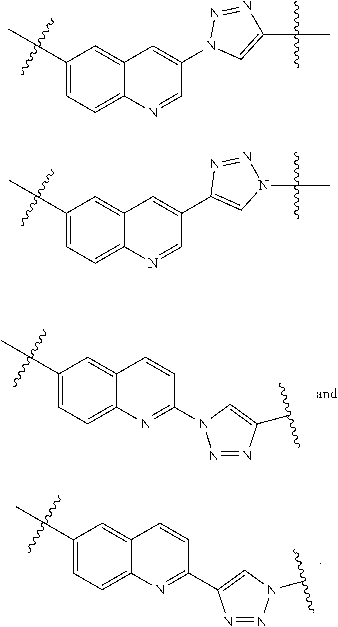 [SMILES: *c1ccc2ncc(-n3cc(*)nn3)cc2c1 ; *c1ccc2ncc(-c3cn(*)nn3)cc2c1 ; *c1ccc2nc(-n3cc(*)nn3)ccc2c1 ; *c1ccc2nc(-c3cn(*)nn3)ccc2c1]

In some embodiments of formula (III)-(XIII), Z² is O.

In some embodiments of formula (III)-(XIII), Z² is S.

In some embodiments of formula (III)-(XIII) Z² is —NR²¹—.

In some embodiments of formula (III)-(XIII), Z² is —C(R²²)₂—, wherein each R²² is independently selected from H, halogen (e.g., F) and optionally substituted (C₁-C₆)alkyl. In some embodiments, Z² is —CH₂—. In some embodiments, Z² is —CHF—. In some embodiments, Z² is —CF₂—.

In some embodiments of formula (III)-(XIII), Z²-A-Z³— is

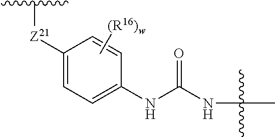

wherein:
Z²¹ is O, S, or —C(R²²)₂—;
R¹⁶ is OH or CH₃; and
w is 0 to 4 (e.g., w is 0, 1, or 2).

In some embodiments, Z²¹ is S or O. In some embodiments, Z²¹ is —CH₂—. In some embodiments, Z²¹ is —CHF—. In some embodiments, Z²¹ is —CF₂—. In some embodiments, R¹⁶ is OH and w is 1. In some embodiments, R¹⁶ is CH₃ and w is 1. In some embodiments, w is 0.

In some embodiments of formula (III)-(XIII), —Z²-A-Z³— is:

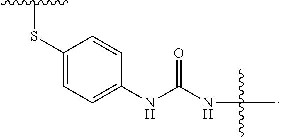 [SMILES: *NC(=O)Nc1ccc(S*)cc1]

In some embodiments of formula (III)-(XII), —Z²-A-Z³— is

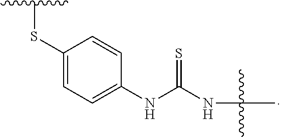 [SMILES: *NC(=S)Nc1ccc(S*)cc1]

In some embodiments of formula (III)-(XIII), —Z²-A-Z³— is

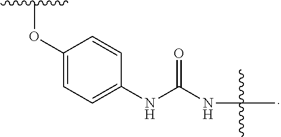 [SMILES: *NC(=O)Nc1ccc(O*)cc1]

In some embodiments of formula (III)-(XIII), —Z²-A-Z³— is

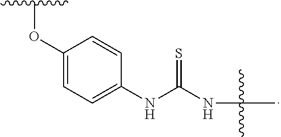 [SMILES: *NC(=S)Nc1ccc(O*)cc1]

In some embodiments of formula (III)-(XIII), —Z²-A-Z³— is

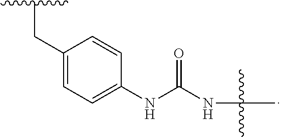 [SMILES: *Cc1ccc(NC(=O)N*)cc1]

In some embodiments of formula (III)-(XIII), —Z²-A-Z³— is

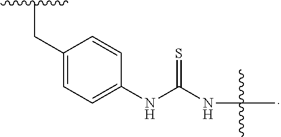 [SMILES: *Cc1ccc(NC(=S)N*)cc1]

### Prodrugs

Aspects of this disclosure include prodrugs of any of the ASGPR and M6PR binding moieties described herein that are incorporated into the compounds and conjugates of this disclosure.

The term “prodrug” refers to an agent which is converted into the drug in vivo by some physiological or chemical process (e.g., a prodrug on being brought to the physiological pH is converted to the desired drug form).

Prodrugs forms of any of the ASGPR or M6PR binding moieties described herein can be useful because, for example, can lead to particular therapeutic benefits as a consequence of an extension of the half-life of the resulting compound or conjugate in the body or a reduction in the active dose required.

Pro-drugs can also be useful in some situations, as they may be easier to administer than the parent drug. They may, for instance, be bioavailable by oral administration whereas the parent drug is not. The pro-drug may also have improved solubility in pharmacological compositions over the parent drug.

Prodrug derivative of a ASGPR or M6PR binding moiety generally includes a promoiety substituent at a suitable labile site of the compound. The promoiety refers to the group that is removed by enzymatic or chemical reactions, when a prodrug is converted to the drug in vivo.

In some embodiments, the promoiety is a group attached via an ester linkage to a hydroxyl group of the compound or drug.

In some embodiments, a prodrug derivative of one or more of the hydroxyl groups of the sugar ring may be incorporated into the compounds. For example, an ester promoiety can be incorporated at one or more of the hydroxyl groups at the 3 and/or 4 positions of the sugar (e.g., as described herein). In some embodiments, the hydroxyl groups at the 3 and 4 positions of the sugar are cyclically linked to form a promoiety (e.g., as described herein).

The terms “linker”, “linking moiety” and “linking group” are used interchangeably and refer to a linking moiety that covalently connects two or more moieties, compounds or other biomolecules, such as ligands and proteins of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes an ethylene glycol, or longer polyethylene glycol (PEG) linking group, e.g., where every third atom of that segment of the linker backbone is substituted with an oxygen. The bonds between backbone atoms of a linker may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol) (also referred to as PEG), ether, thioether, disulfide, amide, carbonate, carbamate, urea, sulfonamide, thiourea, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with a reactive terminus, e.g., suitable for conjugation to a protein of interest. In some instances, the reactive terminus of the linker precursor includes a chemoselective ligation group capable of conjugating to amino acid residue(s) of a polypeptide. In certain instances, the chemoselective ligation group conjugates to a cysteine thiol group, or a lysine sidechain amine group of the polypeptide that is accessible. A variety of conjugation chemistries can be utilized in the conjugates of this disclosure (e.g., as described herein). In some embodiments, the chemoselective ligation group is a thiol-reactive group such as maleimide or dibromomaleimide. In some embodiments, the chemoselective ligation group is an amine-reactive group such as an active ester, e.g., perfluorophenyl ester or tetrafluorophenyl ester, or N-hydroxysuccinimidyl ester (NHS) or sulfo-NHS, or as defined herein.

In certain embodiments of the formula described herein, the linker L includes one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, heteroaryl linkages, or other hetero functional linkages. In certain embodiments, the linker backbone includes one or more of carbon atoms, nitrogen atoms, sulfur atoms, oxygen atoms, and combinations thereof. In certain embodiments, the linker includes one or more of an ether bond, thioether bond, amine bond, amide bond, carbon-carbon bond, carbon-nitrogen bond, carbon-oxygen bond, carbon-sulfur bond, and combinations thereof. In certain embodiments, the linker includes a linear structure. In certain embodiments, the linker includes a branched structure. In certain embodiments, the linker includes a cyclic structure. In certain cases, the linker includes one or more heteroaryl cyclic structures, e.g., a triazole, such as a 1,2,3-triazole.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å.

In certain embodiments, linker L separates X (or Z¹) and Y by a chain of 10 to 100 consecutive atoms. In certain embodiments, linker L separates X (or Z¹) and Y by a chain of 10 to 60 consecutive atoms, by a chain of 12 to 60 consecutive atoms, by a chain of 16 to 50 consecutive atoms, by a chain of 20 to 50 consecutive atoms, by a chain of 30 to 50 consecutive atoms, by a chain of 40 to 50 consecutive atoms.

It is understood that the linker may be considered as connecting directly to a Z¹ group of a ASGPR ligand moiety (X) (e.g., as described herein). In some embodiments of formula II (or any formulae described herein for the ASGPR ligand moiety (X)), the linker may be considered as connecting directly to the Z¹ group. Alternatively, the —Z¹-L¹- group (e.g., as described herein) can be considered part of a linking moiety that connects L to Y. The disclosure is meant to include all such configurations of ASGPR ligand moiety (X) and linker (L).

In some embodiments of formula (I), L is a linker of formula (LXI):

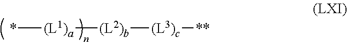

wherein
each L¹ and L³ are independently a linear linking moiety, and L² is a branched linking moiety, wherein L¹ to L³ together provide a linear or branched linker between X and Y;
a, b and c are independently 0 or 1;
* represents the point of attachment of L¹ to X via Z¹; and
** represents the point of conjugation of the linker L to Y;
   wherein:
when n is 1, b is 0 and at least one of a and c is 1; and
when n is 2 or 3, a, b and c are each 1.

In some embodiments of the linker of formula (LXI), n is 1, a is 1, b is 0, and c is 1, such that the linker L is of formula (LXia):

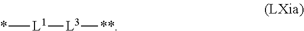

In some embodiments of the linker of formula (LXI), n is 1, a is 1, b is 0, and c is 0, such that the linker L is of formula:

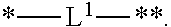

In certain embodiments, the linear linker of formula (LXia) has a backbone of 10 or more consecutive atoms covalently linking X to Y via Z¹, such as a backbone of 12 or more consecutive atoms, 14 or more consecutive atoms, or 16 or more consecutive atoms, and in some cases, up to 100 consecutive atoms. In certain embodiments of formula (LXia), the linear linker separates X (or Z¹) and Y by a chain of 20 to 50 consecutive atoms. In certain embodiments of formula (LXa), the linear linker separates X (or Z¹) and Y by a chain of 30 to 60 consecutive atoms.

In some embodiments of the linker of formula (LXI), n is 2, a is 1, b is 1, and c is 1, such that the linker L is of formula (LXib):

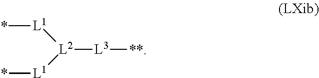

In some embodiments of the linker of formula (LXI), n is 3, a is 1, b is 1, and c is 1, such that the linker L is of formula (LXic):

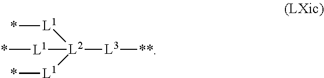

In some embodiments of the linker of any one of formulae (LXI) or (LXia)-(LXic), each L¹ is of formula (LXII):

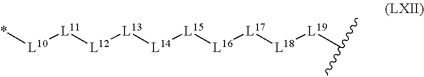

wherein:
L¹⁰ is a linking moiety, and * represents the point of attachment of L¹ to X via Z¹; and
L¹¹ to L¹⁹ are independently absent or a linking moiety, wherein L¹⁰ to L¹⁹ of each L¹ is independently selected from —C₁₋₆-alkylene-, —CF₂—, —C₁₋₁₂-alkylene-, —C₁₋₂₀-alkylene-, —NHCO—C₁₋₆-alkylene-, —CONH—C₁₋₆-alkylene-, —NH—C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —NHCONH—, —NHCSNH—, —CO—, —SO₂—, —O—, —S—, pyrrolidine-2,5-dione, 1,2,3-triazole, —NH—, —N(C₁₋₆-alkyl)-, and —N(CH₃)—, wherein each p is independently 1 to 50, such as 1 to 20, 1 to 12, 1 to 10, 1 to 8, or 1 to 6, e.g., 1, 2, 3, 4, 5 or 6.

In certain embodiments of formula (LXII), the linking moiety L¹ includes a linear backbone of 6 to 40 consecutive atoms, such as 10 to 40, 10 to 30, 16 to 30, or 20 to 30 consecutive atoms. In certain embodiments of formula (LXII), the linking moiety L¹ includes a linear backbone of each L¹ comprises a linear backbone of 6 to 20 consecutive atoms, such as 6 to 16 consecutive atoms, such as 8, 9, 10, 11, 12, 13, 14, 15 or 16 consecutive atoms.

In certain embodiments, the linking moiety of formula (LXII) includes one or repeating ethylene glycol moieties (e.g., —CH₂CH₂O— or —OCH₂CH₂—). In certain cases, the linking moiety of formula (XII) includes 1 to 10 ethylene glycol moieties, such as 1, 2, 3, 4, 5 or 6 ethylene glycol moieties.

In certain embodiments, the linking moiety of formula (LXII) includes one or more triazole (e.g., 1,2,3-triazole) containing linking moieties. It is understood that the triazole may be derived from an azido-alkyne click chemistry and thus have two possible orientations depending on the method of synthesis:

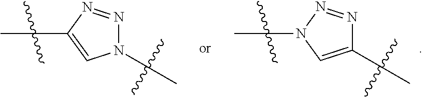 [SMILES: *c1cn(*)nn1 ; *c1cn(*)nn1]

In certain embodiments, the triazole containing linking moiety is:

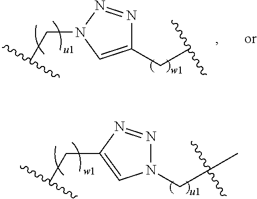

wherein w1 and u1 are independently 0 to 12, such as 0, 1, 2, 3, 4, 5 or 6.

In some embodiments of the linker of formula (LXI), b is 1 and L² is of the formula (LXIIIa) or (LXIIIb):

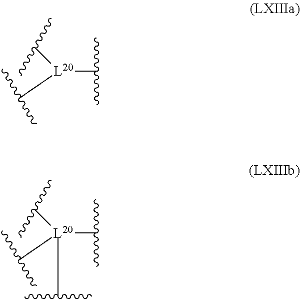

wherein:
L²⁰ is a branched linking moiety including one or more linking moieties independently selected from amino acid residue (e.g., a residue such as Gly, Ala, beta-Ala, Lys, Orn, Asp, Glu, Ser, Cys, or a derivative thereof), —NH—CH[(CH₂)_{q}]₂O— or —NH—C[(CH₂)_{q}]₃O—,

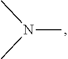

—C₁₋₆-alkylene-, —NHCO—, —CONH—, —NHSO₂—, —SO₂NH—, —CO—, —SO₂—, —O—, —S—, pyrrolidine-2,5-dione, 1,2,3-triazole, —NH—, and -Nine-, —NHC(O)NH—, - NHC(S)NH—, —O(CH₂)ₚ—, and —(OCH₂CH₂)ₚ—;
wherein each p is independently 1 to 50, and q is 1-6.

In some embodiments of the linker of formula (LXI), b is 1 and the linking moiety L² is selected from one of (L2A)-(L2D):

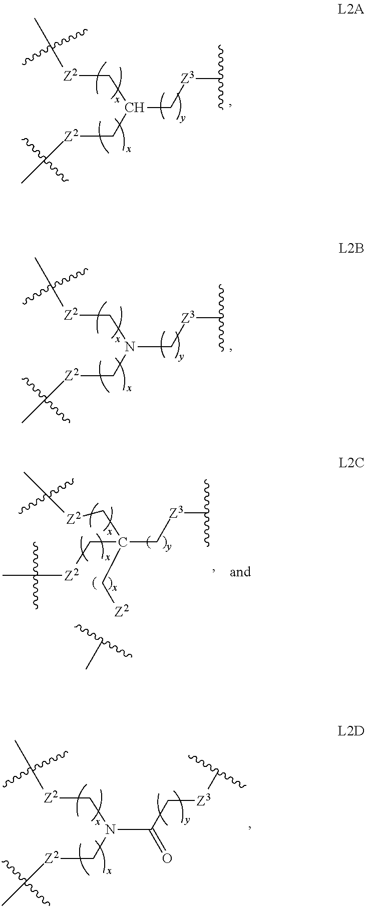

wherein:
each Z² and Z³ is independently absent or selected from —NHCO—, —CONH—, —CO—, —O—, —NH—, and -Nine-;
x is 1 to 12 (e.g., 1 to 6, or 1 to 3); and
y is 0 to 12 (e.g., 1 to 6, or 1 to 3).

In some embodiments of any one of L2A-L2D, Z² is —NHCO—. In some embodiments of any one of L2A-L2D, Z² is —CONH—. In some embodiments of any one of L2A-L2D, Z² is —CO—. In some embodiments of any one of L2A-L2D, Z² is —O—. In some embodiments of any one of L2A-L2D, Z² is —NH—. In some embodiments of any one of L2A-L2D, Z² is -Nine-. In some embodiments of any one of L2A-L2D, Z² is absent.

In some embodiments of any one of L2A-L2D, Z³ is —NHCO—. In some embodiments of any one of L2A-L2D, Z³ is —CONH—. In some embodiments of any one of L2A-L2D, Z³ is —CO—. In some embodiments of any one of L2A-L2D, Z³ is —O—. In some embodiments of any one of L2A-L2D, Z³ is —NH—. In some embodiments of any one of L2A-L2D, Z³ is -Nine-. In some embodiments of any one of L2A-L2D, Z³ is absent.

In some embodiments of L2A, Z² is —O—, y is 0 and the linking moiety is of the structure L2Ai:

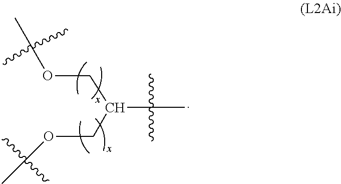

In some embodiments of L2B, Z² is —O— or —CO—, and the linking moiety is of the structure L2Bi or L2Bii:

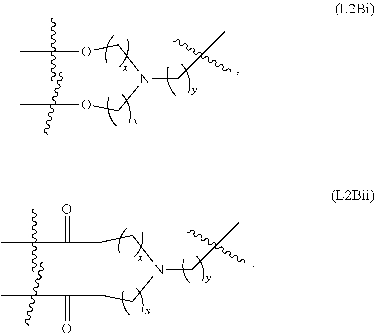

In some embodiments of L2C, Z² is —O—, —CO—, —NHCO—, or —NH—, and the linking moiety is of the structure L2Ci, L2Cii, L2Ciii, or L2Civ:

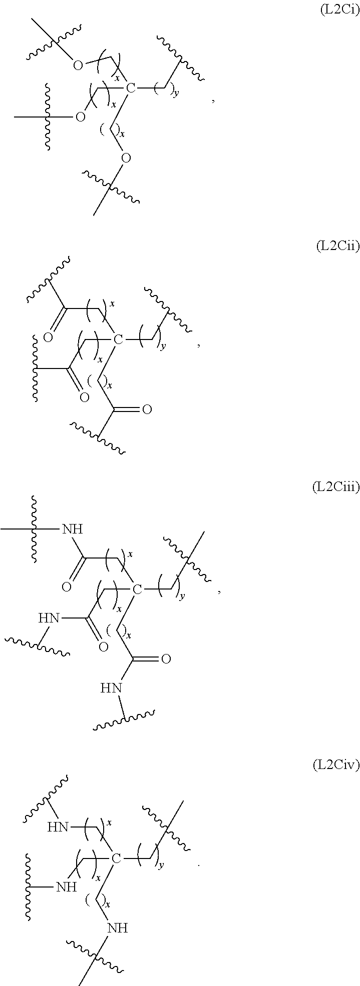

In some embodiments of L2D, Z² is absent and the linking moiety is of the structure L2Di:

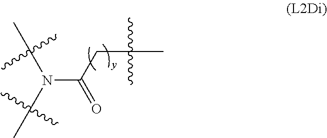

In some embodiments, of any one of formulae L2A-L2Di, x is 1 to 6. In some cases, x is 1 to 3. In some cases, x is 1. In some cases, x is 2. In some cases, x is 3.

In some embodiments of any one of formulae L2A-L2Di, y is 0 to 6. In some cases, y is 0 to 3. In some cases, y is 0. In some cases, y is 1. In some cases, y is 2. In some cases, y is 3.

In certain embodiments of formula (LXI), b is 1 and the linking moiety L² is selected from:

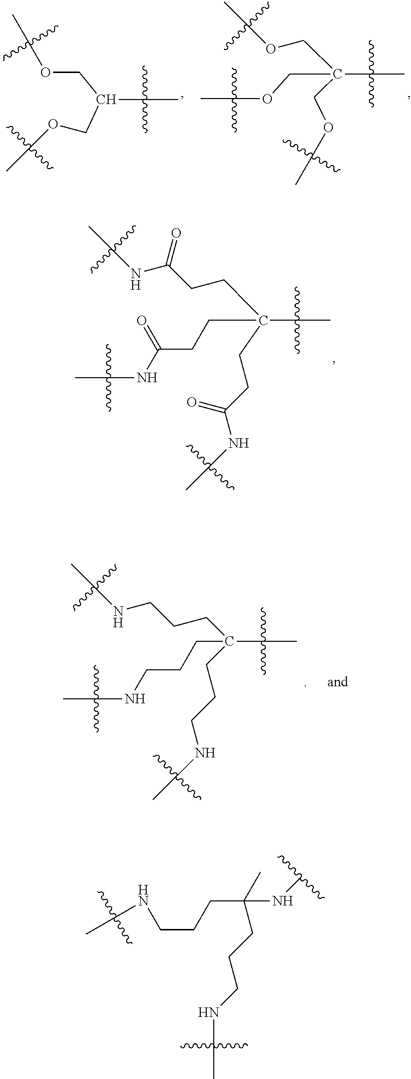

In some embodiments of the linker of formula (LXI), b is 1 and the linking moiety L² is of the formula (LXIV):

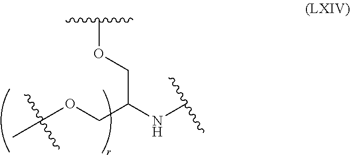

wherein:
r is 1 or 2; and
when n is 2, r is 1,
when n is 3, r is 2.

In some embodiments of the linker of formula (LXI), b is 1 and the linking moiety L² is of the formula (LXva) or (LXVb):

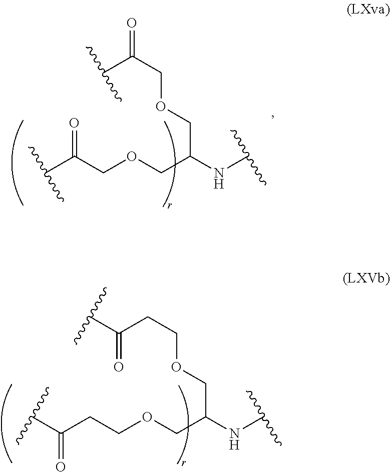

wherein:
r is 1 or 2; and
when n is 2, r is 1,
when n is 3, r is 2.

In some embodiments L² is of formula (LXIIIa) or (LXIIIb) and L² includes two 2 or more amino acid residues (e.g., 3 or more, or 4 or more amino acid residues, linear or dendrimer). In some embodiments, L² includes 4 or more amino acid residues that are branched linking moieties selected from Lys, Orn, Asp, Glu, Ser, and Cys (e.g., where the sidechain, amino and carboxylic acid are each linked to an adjacent moiety).

In some embodiments of the linker of any one of formulae (LXI) or (LXa)-(LXc), each L³ is of the formulae (LXVI):

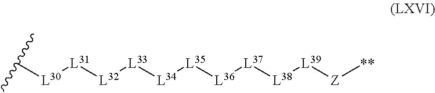

wherein:
L³⁰ to L³⁹ are independently absent or a linking moiety; and
Z is a residual moiety resulting from the covalent linkage of a chemoselective ligation group of the linker to a compatible group of Y;
wherein L³⁰ to L³⁹ are each independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, - NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —NHCONH—, —NHCSNH—, —CO—, —SO₂—, —O—, —S—, pyrrolidine-2,5-dione, 1,2,3-triazole, —NH—, and —NMe-, wherein each p is independently 1 to 50.

In certain embodiments, the linking moiety of formula (LXVI) includes a linear backbone of 6 to 40 consecutive atoms, such as 10 to 40, 10 to 30, or 20 to 30 consecutive atoms.

In certain embodiments, the linking moiety of formula (LXVI) includes repeating ethylene glycol moieties (e.g., —CH₂CH₂O— or —OCH₂CH₂—). In certain cases, the linking moiety of formula (XVI) includes 2 to 20 ethylene glycol moieties, such as 2 to 15, 2 to 10, 3 to 20, 3 to 15, 3 to 10, 4 to 15, 5 to 15 or 5 to 10 ethylene glycol moieties. In some instances, the linking moiety of formula (XVI) includes 2 or more ethylene glycol moieties, such as 3 or more, 4 or more, 5 or more, 6 or more, 7 or more, or even more ethylene glycol moieties.

In certain embodiments, the linking moiety of formula (LXVI) includes one or more triazole linking moieties. In some instances, the linker includes one or more 1,2,3-triazole linking moieties. In certain cases, the one or more 1,2,3-triazole moieties is selected from one of the following structures:

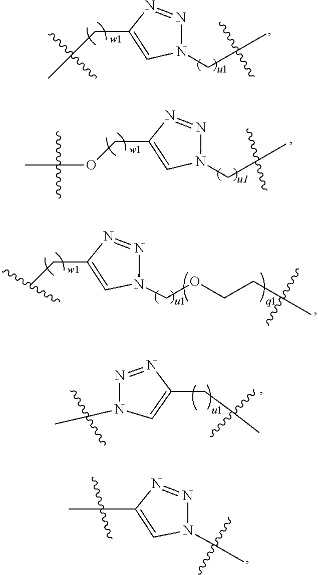

wherein w1, u1 and q1 are independently 1 to 25 (e.g., 1 to 12, such as 1 to 6).

In certain embodiments, the linking moiety L³ includes (C₁₀-C₂₀-alkylene (e.g., C₁₂-alkylene), or —(OCH₂CH₂)ₚ—, where p is 1 to 25, such as 3 to 25, 5 to 24, 7 to 25, 10 to 25, 15 to 25 or 20 to 24.

In some embodiments, the linker L is of formula LXVII:

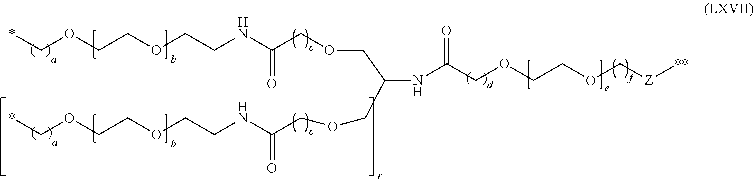

wherein:
a is 0 to 12 (e.g., 2 to 6, or 2, or 3);
b is 1 to 6 (e.g., 1, 2, or 3);
c is 1 to 6 (e.g., 1, 2, or 3);
r is 1 or 2;
d is 1 to 6 (e.g., 1, 2, or 3);
e is b is 1 to 6 (e.g., 1, 2, or 3);
f is 1 to 6 (e.g., 1, 2, or 3);
Z is a residual moiety resulting from the covalent linkage of a chemoselective ligation group (e.g., as described herein) of a linker precursor to a compatible group of Y.

In some embodiments of the formula LXVII, Z is a residual moiety resulting from the covalent linkage (e.g., via a thioether bond) of a thiol-reactive chemoselective ligation group to one or more cysteine residue(s) of Y. In some embodiments, the thiol-reactive chemoselective ligation group includes maleimide, bromomaleimide, haloacetamide, vinyl sulfone, or thiolactone. In some embodiments, the thiol-reactive group is selected from one of the following structures:

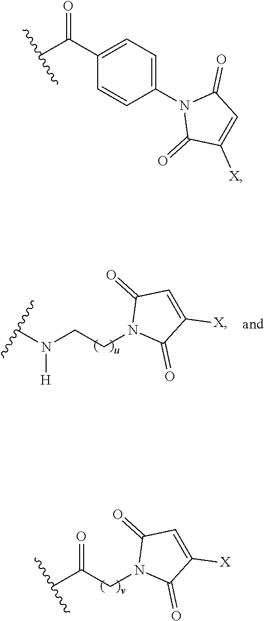

wherein:
u is 1 to 11 (e.g., 1 to 5);
v is 1 to 11 (e.g., 1 to 5); and
X is H or Br.

In some embodiments of formula LXVII, Z is a residual moiety resulting from the covalent linkage (e.g., via an amide bond) of an amine-reactive chemoselective ligation group to one or more lysine residue(s) of Y. In some embodiments, the amine-reactive chemoselective ligation group includes an active ester (e.g., N-hydroxysuccinimidyl (NHS) ester, sulfo-NHS ester, pentafluorophenyl (PFP) ester, tetrafluorophenyl (TFP) ester, or the like).

In some embodiments, the linker L includes one of (LXVIIIa)-(LXVIIIc):

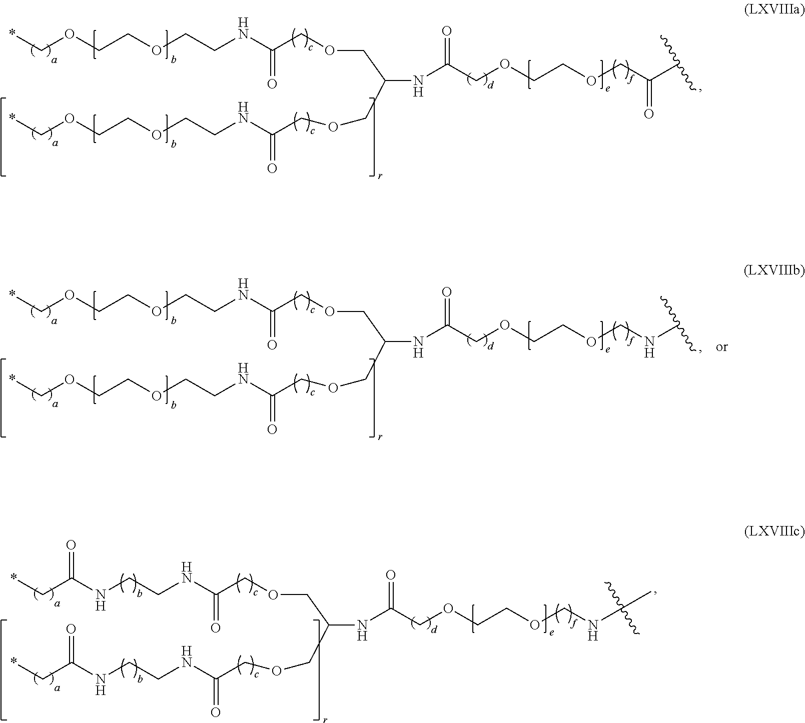

wherein:
a is 0 to 12 (e.g., 2 to 6, or 2, or 3);
b is 1 to 6 (e.g., 1, 2, or 3);
c is 1 to 6 (e.g., 1, 2, or 3);
r is 1 or 2;
d is 1 to 6 (e.g., 1, 2, or 3);
   e is b is 1 to 6 (e.g., 1, 2, or 3); and
f is 1 to 6 (e.g., 1, 2, or 3).

In some embodiments of any one of formulae (LXVII) or (LXVIIIa)-(LXVIIIc), a is 2 to 6, such as 2 to 3. In some embodiments, a is 2. In some embodiments, a is 3. In some embodiments, a is 4. In some embodiments, a is 5. In some embodiments a is 6.

In some embodiments of any one of formulae (LXVII) or (LXVIIIa)-(LXVIIIc), b is 1 to 4, such as 1 to 3. In some embodiments, b is 1. In some embodiments, b is 2. In some embodiments, b is 3.

In some embodiments of any one of formulae (LXVII) or (LXVIIIa)-(LXVIIIc), c is 1 to 4, such as 1 to 3. In some embodiments, c is 1. In some embodiments, c is 2. In some embodiments, c is 3.

In some embodiments of any one of formulae (LXVII) or (LXVIIIa)-(LXVIIIc), r is 1. In some embodiments, r is 2.

In some embodiments of any one of formulae (LXVII) or (LXVIIIa)-(LXVIIIc), d is 1 to 4, such as 1 to 3. In some embodiments, d is 1. In some embodiments, d is 2. In some embodiments, d is 3.

In some embodiments of any one of formulae (LXVII) or (LXVIIIa)-(LXVIIIc), e is 1 to 5, such as 1 to 3. In some embodiments, e is 1. In some embodiments, e is 2. In some embodiments, e is 3. In some embodiments, e is 4. In some embodiments, e is 5.

In some embodiments of any one of formulae (LXVII) or (LXVIIIa)-(LXVIIIc), f is 1 to 4, such as 1 to 3. In some embodiments, f is 1. In some embodiments, f is 2. In some embodiments, f is 3.

In some embodiments of any one of formulae (LXVII) or (LXVIIIa)-(LXVIIIc), a is 1-4; b is 1-4; c is 1-3; r is 1; d is 1-3; e is 1-6; and f is 1-3.

In some embodiments of any one of formulae (LXVII) or (LXVIIIa)-(LXVIIIc), a is 1-4; b is 1-4; c is 1-3; r is 2; d is 1-3; e is 1-6; and f is 1-3.

In some embodiments of any one of formulae (LXVII) or (LXVIIIa)-(LXVIIIc), a is 2; b is 1; c is 2; r is 1; d is 2; e is 3; and f is 2.

In some embodiments of any one of formulae (LXVII) or (LXVIIIa)-(LXVIIIc), a is 2; b is 1; c is 2; r is 2; d is 2; e is 3; and f is 2.

In some embodiments of any one of formulae (LXVII) or (LXVIIIa)-(LXVIIIc), a is 4; b is 1; c is 2; r is 1; d is 2; e is 3; and f is 2.

In some embodiments of any one of formulae (LXVII) or (LXVIIIa)-(LXVIIIc), a is 4; b is 1; c is 2; r is 2; d is 2; e is 3; and f is 2.

In some embodiments of any one of formulae (LXVII) or (LXVIIIa)-(LXVIIIc), a is 2; b is 2; c is 2; r is 1; d is 2; e is 3; and f is 2.

In some embodiments of any one of formulae (LXVII) or (LXVIIIa)-(LXVIIIc), a is 2; b is 2; c is 2; r is 2; d is 2; e is 3; and f is 2.

In some embodiments of any one of formulae (LXVII) or (LXVIIIa)-(LXVIIIc), a is 0; b is 3; c is 2; r is 2; d is 2; e is 3; and f is 2.

In some embodiments of any one of formulae (LXVII) or (LXVIIIa)-(LXVIIIc), a is 2; b is 4; c is 2; r is 2; d is 2; e is 3; and f is 2.

In some embodiments of any one of formulae (LXVII) or (LXVIIIa)-(LXVIIIc), a is 2; b is 4; c is 2; r is 1; d is 2; e is 3; and f is 2.

In some embodiments, the linker L includes L^{A}:

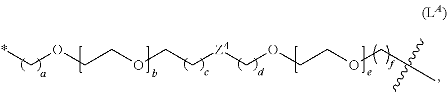

wherein:
   Z⁴ is selected from —NHC(O)NH—, —NHC(O)—, —C(O)NH—, —O—, —NH—;
a is 0 to 12 (e.g., 2 to 6, or 2, or 3);
   b is 1 to 6 (e.g., 1, 2, or 3);
   c is 1 to 6 (e.g., 1, 2, or 3);
   d is 1 to 6 (e.g., 1, 2, or 3);
e is b is 1 to 6 (e.g., 1, 2, or 3); and
   f is 1 to 6 (e.g., 1, 2, or 3).

In some embodiments of L^{A}, Z⁴ is —NHC(O)NH—. In some cases, Z⁴ is —NHC(O)—. In some cases, Z⁴ is —C(O)NH—. In some cases, Z⁴ is —O—. In some cases, Z⁴ is —NH—.

In some embodiments of L^{A}, a is 1-4; b is 1-4; c is 1-3; d is 1-3; e is 1-6; and f is 1-3. In some embodiments, a is 4; b is 1; c is 2; d is 2; e is 5; and f is 2.

In some embodiments, Z⁴ is —NHC(O)NH— and a is 1-4; b is 1-4; c is 1-3; r is 1; d is 1-3; e is 1-6; and f is 1-3. In some embodiments, Z⁴ is —NHC(O)— and a is 1-4; b is 1-4; c is 1-3; r is 1; d is 1-3; e is 1-6; and f is 1-3.

In some embodiments, the linker L includes L^{B}:

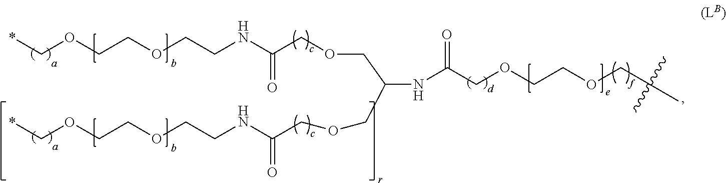

wherein:
a is 0 to 12 (e.g., 2 to 6, or 2, or 3);
   b is 1 to 6 (e.g., 1, 2, or 3);
   c is 1 to 6 (e.g., 1, 2, or 3);
   r is 1 or 2;
   d is 1 to 6 (e.g., 1, 2, or 3);
e is b is 1 to 6 (e.g., 1, 2, or 3); and
   f is 1 to 6 (e.g., 1, 2, or 3).

In some embodiments of L^{B}, a is 1-4; b is 1-4; c is 1-3; r is 1; d is 1-3; e is 1-6; and f is 1-3. In some embodiments, a is 4; b is 1; c is 2; r is 1; d is 2; e is 5; and f is 2. In some embodiments, a is 2; b is 1; c is 2; r is 1; d is 2; e is 3; and f is 2. In some embodiments, a is 4; b is 1; c is 2; r is 1; d is 2; e is 3; and f is 2. In some embodiments, a is 1; b is 2; c is 2; r is 1; d is 2; e is 3; and f is 2. In some embodiments, a is 0; b is 3; c is 2; r is 1; d is 2; e is 3; and f is 2.

In some embodiments of L^{B}, a is 1-4; b is 1-4; c is 1-3; r is 2; d is 1-3; e is 1-6; and f is 1-3. In some embodiments, a is 2; b is 1; c is 2; r is 2; d is 2; e is 3; and f is 2. In some embodiments, a is 4; b is 1; c is 2; r is 2; d is 2; e is 3; and f is 2. In some embodiments, a is 1; b is 2; c is 2; r is 2; d is 2; e is 3; and f is 2. In some embodiments, a is 0; b is 3; c is 2; r is 2; d is 2; e is 3; and f is 2.

In some embodiments, the linker L includes L^{C}:

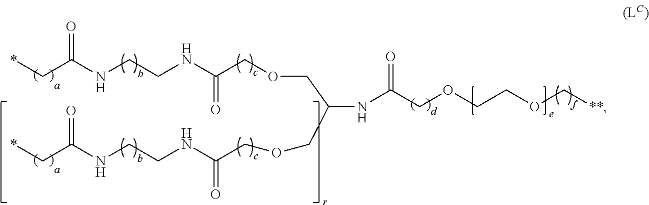

wherein:
a is 0 to 12 (e.g., 1 to 6, 2 to 6, or 2 or 3);
   b is 1 to 6 (e.g., 1 to 4, such as 1, 2, or 3);
   c is 1 to 6 (e.g., 1 to 3, such as 1, 2, or 3);
   r is 1 or 2;
   d is 1 to 6 (e.g., 1 to 3, such as 1, 2 or 3);
e is b is 1 to 6 (e.g., 1, 2, or 3);
   f is 1 to 6 (e.g., 1 to 3, such as 1, 2, or 3).

In some embodiments of L^{c}, a is 1-4; b is 1-4; c is 1-3; r is 1; d is 1-3; e is 1-6; and f is 1-3. In some embodiments, a is 2; b is 4; c is 2; r is 1; d is 2; e is 5; and f is 2.

In some embodiments of L^{c}, a is 1-4; b is 1-4; c is 1-3; r is 2; d is 1-3; e is 1-6; and f is 1-3. In some embodiments, a is 2; b is 4; c is 2; r is 2; d is 2; e is 5; and f is 2.

In certain embodiments of the ASGPR binding moiety (X) as described herein, —Z¹— is linked to an -L¹- moiety (e.g., of the linker as described herein). In some embodiments, the subject compounds comprise a —Z¹-L¹- moiety comprising a linking moiety selected from:

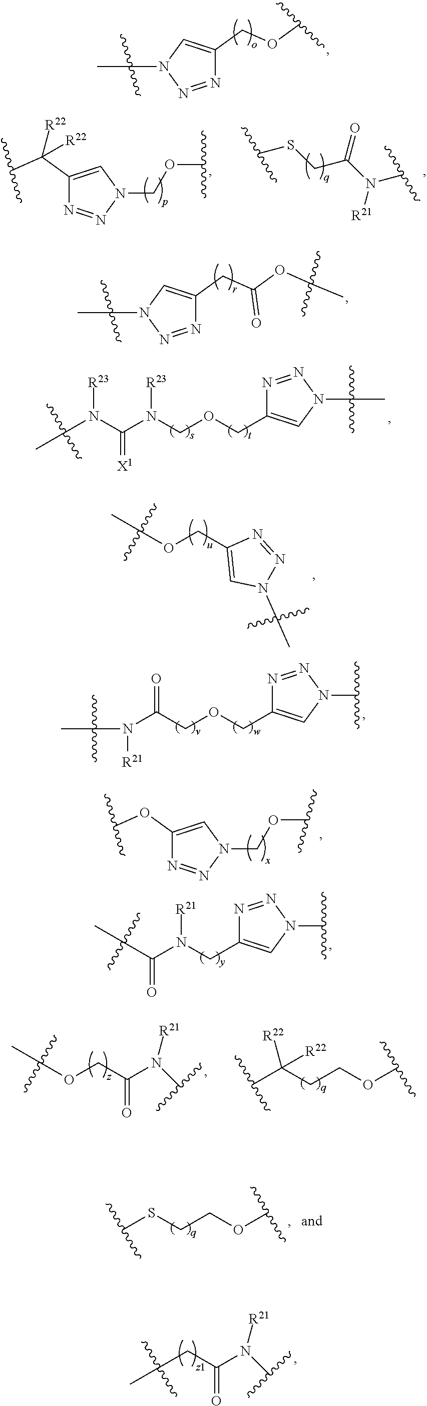

wherein each R²¹ is independently selected from H, and optionally substituted (C₁-C₆)alkyl; each R²² is independently selected from H, halogen (e.g., F) and optionally substituted (C₁-C₆)alkyl; and o, p, q, r, s, t, u, v, w, x, y, z and z1 are each independently 1 to 6.

In certain embodiments, the Z¹-L¹- group is

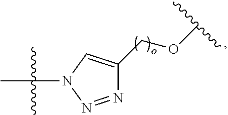

and o is 1 or 2.

In certain embodiments, the Z¹-L¹- group is

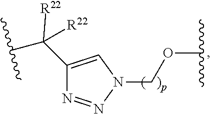

each R²² is H, and p is 1 or 2.

In certain embodiments, the Z¹-L¹- group is

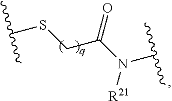

where q is 1-3.

In certain embodiments, the Z¹-L¹- group is

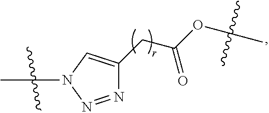

where r is 1-3.

In certain embodiments, the Z¹-L¹- group is

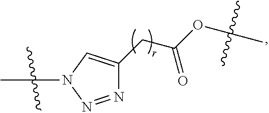

N where r is 1-3.

In certain embodiments, the Z¹-L¹- group is

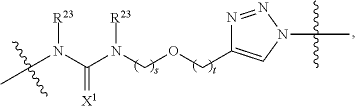

where s and t are each independently 1-3.

In certain embodiments, the Z¹-L¹- group is

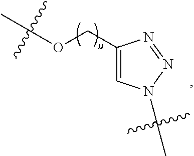

where u is 1-3.

In certain embodiments, the Z¹-L¹- group is

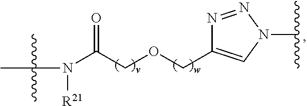

where v and w are each independently is 1-3.

In certain embodiments, the Z¹-L¹- group is

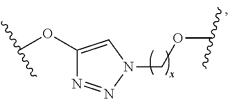

where x is 0-3.

In certain embodiments, the Z¹-L¹- group is

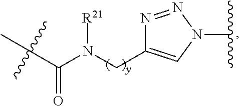

where y is 1-3.

In certain embodiments, the Z¹-L¹- group is

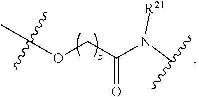

where R²¹ is H, and z is 1-4.

In certain embodiments, the Z¹-L¹- group is

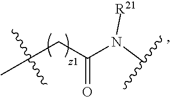

where R²¹ is H, and z1 is 1-4.

In certain embodiments, the Z¹-L¹- group is

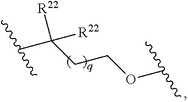

where each R²² is H, and q is 1-3.

In certain embodiments, the Z¹-L¹- group is

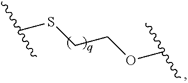

where q is 1-3.

In certain embodiments, the subject compounds comprise a —Z¹-L- group comprising a linking moiety selected from:

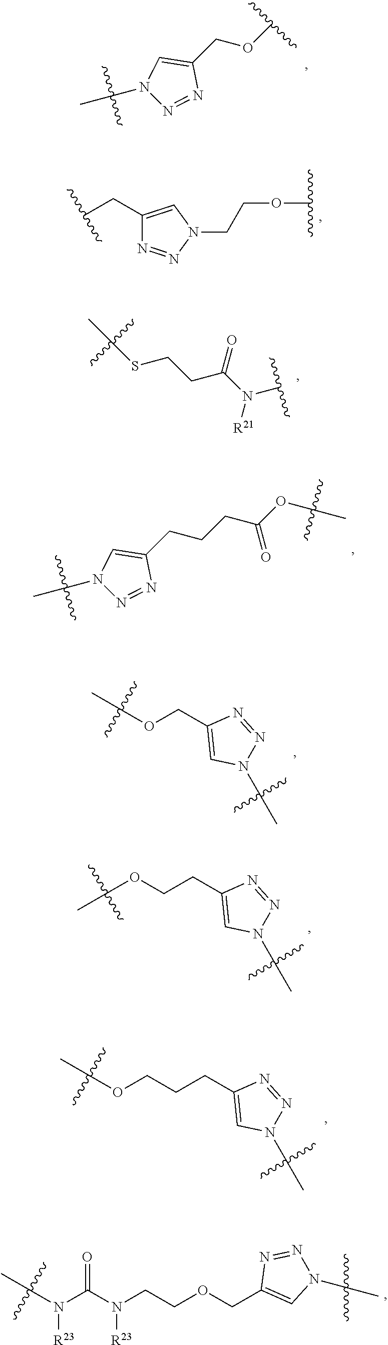

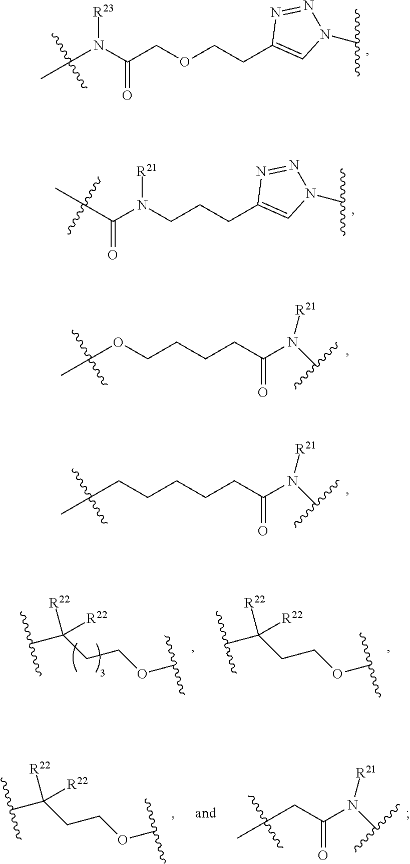

where R²¹ is independently selected from H, and optionally substituted (C₁-C₆)alkyl (e.g., methyl); and each R²² is independently selected from H, halogen (e.g., F) and optionally substituted (C₁-C₆)alkyl (e.g., methyl). In certain embodiments, R²¹ is H. In certain embodiments, each R²² is H.

In certain embodiments, the —Z¹-L¹- group is

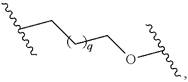

where q is 1-3. In certain cases, q is 1. In certain cases, q is 2. In certain cases, q is 3.

In certain embodiments, the —Z¹-L¹- group is

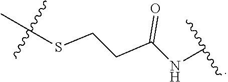 [SMILES: *NC(=O)CCS*]

In certain embodiments, —Z¹-L¹- includes an optionally substituted —NH-heteroarylene-. In certain embodiments, the heteroarylene is a triazole. In certain cases, the heteroarylene is pyridine. In certain cases, the heteroarylene is pyrimidine. In certain cases, the heteroarylene is thiadiazole.

In certain embodiments, the —Z¹-L¹- includes a group selected from:

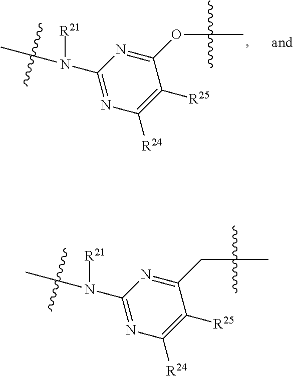

wherein R²⁴ and R²⁵ are each independently selected from H, optionally substituted C₍₁₋₆₎-alkyl, optionally substituted fluoroalkyl, and halogen; and each R²¹ is independently selected from H, optionally substituted (C₁-C₆)alkyl, and optionally substituted alkanoyl. In certain cases, R²¹ is H. In certain cases, R²⁴ is C₍₁₋₃₎-alkyl, or C₍₁₋₃₎-fluoroalkyl. In some cases, the fluoroalkyl is CF₃. In certain cases, R²⁵ is C₍₁₋₃₎-alkyl, or C₍₁₋₃₎-fluoroalkyl. In some cases, the fluoroalkyl is CF₃.

In some embodiments, the linker includes a polypeptide scaffold where some or all of the sidechain groups of the amino acid residues of such a polypeptide scaffold have been modified to attach a X binding moiety (e.g., as described herein). It is understood that X binding moieties (e.g., as described herein) can be conjugated to amino acid residues, such as Asp, Lys, Om, Glu, and Ser, of a polypeptide containing linker via a convenient conjugation chemistry. In some embodiments, the linker contains a polylysine polypeptide. In some embodiments, the linker contains a polyornithine polypeptide. In some embodiments, the linker contains a polyserine polypeptide. In some embodiments, the linker contains a polyaspartate polypeptide. The polypeptide backbone of such a linker can be a randomly polymerized polymer having an average length, or a polymer of defined length prepared e.g., in a controlled stepwise fashion. In some cases, the polypeptide linker has a length of 10-100 amino acid residues, such as 20-90, or 20-50 amino acid residues. In some embodiments, the N-terminal or C-terminal of the polypeptide linker is modified to include a linking moiety to an additional X binding moiety (e.g., as described herein). In some embodiments, the N-terminal or C-terminal of the polypeptide linker segment is modified with one or more linking moieties (e.g., as described herein) suitable for attachment to a protein construct (Y) including a polypeptide that specifically binds an autoantibody.

In some embodiments, the terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n¬butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a cell surface receptor (e.g., ASGPR). When Y is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, heteroaryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure. In certain cases, the linker comprises one or more heteroaryl cyclic structures, e.g., a triazole, such as a 1,2,3-triazole.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to X, an optionally substituted heteroarylene linked to X, an alkylene group linked to X, or a heteroatom linked to X. In certain embodiments, L is a linker between about 10 Å and about 500 Å, which comprises an optionally substituted arylene linked to X, or optionally substituted heteroarylene linked to X, an alkylene group linked to X, or a heteroatom linked to X. In certain embodiments, L is a linker between about 10 Å and about 400 Å, which comprises an optionally substituted arylene linked to X, or optionally substituted heteroarylene linked to X, an alkylene group linked to X, or a heteroatom linked to X. In certain embodiments, L is a linker between about 10 Å and about 200 Å, which comprises an optionally substituted arylene linked to X, or optionally substituted heteroarylene linked to X, an alkylene group linked to X, or a heteroatom linked to X.

In certain embodiments, linker L separates X and Y (or Z¹) by a chain of 4 to 500 consecutive atoms. In certain embodiments, linker L separates X and Y (or Z¹) by a chain of 4 to 50 consecutive atoms. In certain embodiments, linker L separates X and Y (or Z¹) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z¹) by a chain of 6 to 50 consecutive atoms. In certain embodiments, linker L separates X and Y (or Z¹) by a chain of 11 to 50 consecutive atoms. In certain embodiments, linker L separates X and Y (or Z¹) by a chain of 16 to 50 consecutive atoms. In certain embodiments, linker L separates X and Y (or Z¹) by a chain of 21 to 50 consecutive atoms. In certain embodiments, linker L separates X and Y (or Z¹) by a chain of 26 to 50 consecutive atoms. In certain embodiments, linker L separates X and Y (or Z¹) by a chain of 31 to 50 consecutive atoms. In certain embodiments, linker L separates X and Y (or Z¹) by a chain of 36 to 50 consecutive atoms. In certain embodiments, linker L separates X and Y (or Z¹) by a chain of 41 to 50 consecutive atoms. In certain embodiments, linker L separates X and Y (or Z¹) by a chain of 46 to 50 consecutive atoms.

In certain embodiments, linker L separates X and Y (or Z¹) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L is a chain of 5 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 7 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 10 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 15 to 400 consecutive atoms separating X and Y (or Z¹) and which comprises an optionally substituted arylene linked to X, optionally substituted heteroarylene linked to X, optionally substituted alkylene linked to X, or heteroatom linked to X.

In certain embodiments, linker L is a chain of 5 to 500 consecutive atoms separating X and Y (or Z¹) and which comprises an alkylene, a heteroatom, or optionally substituted heteroarylene linked to X. In certain embodiments, linker L is a chain of 7 to 500 consecutive atoms separating X and Y (or Z¹) and which comprises an alkylene, a heteroatom, or optionally substituted heteroarylene linked to X. In certain embodiments, linker L is a chain of 10 to 500 consecutive atoms separating X and Y (or Z¹) and which comprises an comprises an alkylene, a heteroatom, or optionally substituted heteroarylene linked to X. In certain embodiments, linker L is a chain of 15 to 400 consecutive atoms separating X and Y (or Z¹) and which comprises an alkylene, a heteroatom, or optionally substituted heteroarylene linked to X.

In certain embodiments, linker L is a chain of 5 to 500 consecutive atoms separating X and Y (or Z¹) and which comprises an optionally substituted triazole linked to X. In certain embodiments, linker L is a chain of 7 to 500 consecutive atoms separating X and Y (or Z¹) and which comprises an optionally substituted triazole linked to X. In certain embodiments, linker L is a chain of 10 to 500 consecutive atoms separating X and Y (or Z¹) and which comprises an optionally substituted triazole linked to X. In certain embodiments, linker L is a chain of 15 to 400 consecutive atoms separating X and Y (or Z¹) and which comprises an optionally substituted triazole linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted alkylene linked to X, or a heteroatom linked to X.

It is understood that the linker may be considered as connecting directly to a Z¹ group of X (e.g., as described herein). In some embodiments of any of formulae (Ia)-(Ip), the linker may be considered as connecting directly to the Z¹ group. Alternatively, the —Z¹-L¹- group (e.g., as described herein) can be considered part of a linking moiety that connects L to Y. The disclosure is meant to include all such configurations of X and linker (L).

In some embodiments of formula (I), L is a linker of formula (II′″).

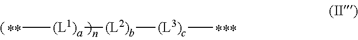

wherein
L¹ and L³ are independently a linker, and L² is a branched linking moiety, wherein L¹ to L³ together provide a linear or branched linker between X and Y;
a, b and c are independently 0 or 1;
** represents the point of attachment to L¹ of X via Z¹; and
*** represents the point of attachment to Y;
   wherein:
when n is 1, a is 1, and b is 0;
when n is >1, a is 1, and b is 1.

In certain embodiments of the linker of formula (II), L¹ to L³ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, - NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non- naturally occurring amino acid residue), —NH—, and —NMe-, wherein each p is independently 1 to 50.

In certain embodiments of the linker of formula (II), any of L¹-L³ comprises repeating ethylene glycol moieties (e.g., —CH₂CH₂O— or —OCH₂CH₂—). In certain cases, the linker of formula (II) comprises 1 to 25 ethylene glycol moieties, such as 3 to 25, 5 to 25, 7 to 25, 10 to 25, 15 to 25, 17 to 25, 20 to 25 or 22 to 25 ethylene glycol moieties. In some instances, the linker of formula (II) comprises 3 or more ethylene glycol moieties, such as 5 or more, 7 or more, 10 or more, 15 or more, 20 or more, or even more ethylene glycol moieties.

In certain embodiments of the linker of formula (II), any of L¹-L³ comprises one or more triazole linking moieties. In some instances, the linker comprises one or more 1,2,3-triazole linking moieties. In certain cases, the one or more 1,2,3-triazole moieties is selected from one of the following structures:

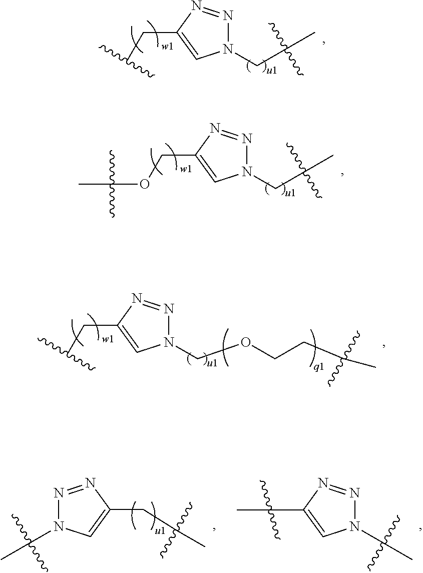

wherein w1, u1 and q1 are independently 1 to 25 (e.g., 1 to 12, such as 1 to 6).

In certain embodiments of the linker of formula (II), n is 1, such that b is 0, and the linker is of the formula (IIa′″):

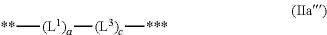

wherein
L¹ and L³ are independently a linker (e.g., as described herein), wherein L¹ to L³ together provide a linear linker between X and Y;
a is 1;
c is 0 or 1;
** represents the point of attachment to L¹ of X via Z¹; and
*** represents the point of attachment to Y.

In certain embodiments of the linker of formula (IIa), the linear linker has a backbone of 20 or more consecutive atoms covalently linking X to Y via Z¹, such as a backbone of 25 or more consecutive atoms, or 30 or more consecutive atoms, and in some cases, up to 100 consecutive atoms. In certain embodiments of formula (IIa), the linear linker separates X and Y (or Z¹) by a chain of 20 to 50 consecutive atoms. In certain embodiments, the linear linker separates X and Y (or Z¹) by a chain of 21 to 50 consecutive atoms, by a chain of 22 to 50 consecutive atoms, by a chain of 23 to 50 consecutive atoms, by a chain of 24 to 50 consecutive atoms, by a chain of 25 to 50 consecutive atoms, by a chain of 26 to 50 consecutive atoms, by a chain of 27 to 50 consecutive atoms, by a chain of 28 to 50 consecutive atoms, or by a chain of 29 to 50 consecutive atoms. In certain embodiments of formula (IIa), the linear linker separates X and Y (or Z¹) by a chain of 30 to 60 consecutive atoms. In certain embodiments, the linear linker separates X and Y (or Z¹) by a chain of 31 to 60 consecutive atoms. In certain embodiments, the linear linker separates X and Y (or Z¹) by a chain of 32 to 60 consecutive atoms. In certain embodiments, the linear linker separates X and Y (or Z¹) by a chain of 33 to 60 consecutive atoms. In certain embodiments, the linear linker separates X and Y (or Z¹) by a chain of 34 to 60 consecutive atoms. In certain embodiments, the linear linker L separates X and Y (or Z¹) by a chain of 35 to 50 consecutive atoms. In certain embodiments, the linear linker L separates X and Y (or Z¹) by a chain of 36 to 50 consecutive atoms. In certain embodiments, the linear linker L separates X and Y (or Z¹) by a chain of 41 to 50 consecutive atoms. In certain embodiments, the linear linker L separates X and Y (or Z¹) by a chain of 46 to 50 consecutive atoms.

In certain other embodiments of formula (II′″), n is 2 or more, such that L¹ to L³ together provide a branched linker between X and Y.

In some embodiments of the linker of formula (XI), n is 1, a is 1, b is 0, and c is 0, such that the linker L is of formula:

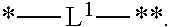

In certain embodiments, the linear linker of formula (Xia) has a backbone of 10 or more consecutive atoms covalently linking X to Y via Z¹, such as a backbone of 12 or more consecutive atoms, 14 or more consecutive atoms, or 16 or more consecutive atoms, and in certain embodiments, up to 100 consecutive atoms. In certain embodiments of formula (Xia), the linear linker separates X (or Z¹) and Y by a chain of 20 to 50 consecutive atoms. In certain embodiments of formula (Xa), the linear linker separates X (or Z¹) and Y by a chain of 30 to 60 consecutive atoms.

In some embodiments of the linker of formula (XI), n is 2, a is 1, b is 1, and c is 1, such that the linker L is of formula (Xib):

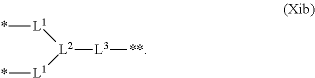

In some embodiments of the linker of formula (XI), n is 3, a is 1, b is 1, and c is 1, such that the linker L is of formula (Xic):

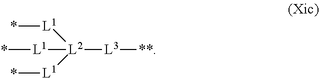

In some embodiments of the linker of any one of formulae (XI) or (Xia)-(Xic), each L¹ is of formula (XII):

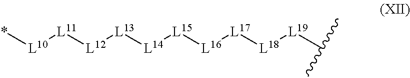

wherein:
L¹⁰ is a linking moiety, and * represents the point of attachment of L¹ to X via Z¹; and
L¹¹ to L¹⁹ are independently absent or a linking moiety,
wherein L¹⁰ to L¹¹ of each L¹ is independently selected from —C₁₋₆-alkylene-, —C₁₋₁₂-alkylene-, —C₁₋₂₀-alkylene-, —NHCO—C₁₋₆-alkylene-, —CONH—C₁₋₆-alkylene-, —NH—C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, — (OCH₂CH₂)ₚ—, —NHCO—, —CONH—, —NHSO₂—, —SO₂NH—, —NHCONH—, —NHCSNH—, —CO—, —SO₂—, —O—, —S—, arylene, heteroarylene, heteroalkylene, cycloalkylene, —NH—, —N(C₁₋₆-alkyl)-, and —N(CH₃)—, wherein each L¹⁰ to L¹⁹ of each L¹ is independently optionally substituted with one or more halo (e.g., 1 to 3, or 1 to 5); and p is independently 1 to 50, such as 1 to 20, 1 to 12, 1 to 10, 1 to 8, or 1 to 6, e.g., 1, 2, 3, 4, 5 or 6.

In some embodiments of the linker of any one of formulae (XI) or (Xia)-(Xic), each L¹ is of formula (XII):

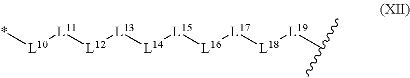

wherein:
L¹⁰ is a linking moiety, and * represents the point of attachment of L to X via Z¹; and
L¹¹ to L¹⁹ are independently absent or a linking moiety,
wherein L¹⁰ to L¹⁹ of each L¹ is independently selected from —C₁₋₆-alkylene-, —CF₂—, —C₁₋₁₂-alkylene-, —C₁₋₂₀-alkylene-, —NHCO—C₁₋₆-alkylene-, —CONH—C₁₋₆-alkylene-, —NH—C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —NHCONH—, —NHCSNH—, —CO—, —SO₂—, —O—, —S—, pyrrolidine-2,5-dione, 1,2,3-triazole, —NH—, —N(C₁₋₆-alkyl)-, and —N(CH₃)—, wherein each p is independently 1 to 50, such as 1 to 20, 1 to 12, 1 to 10, 1 to 8, or 1 to 6, e.g., 1, 2, 3, 4, 5 or 6.

In certain embodiments of formula (XII), the linking moiety L¹ includes a linear backbone of 6 to 40 consecutive atoms, such as 10 to 40, 10 to 30, 16 to 30, or 20 to 30 consecutive atoms. In certain embodiments of formula (XII), the linking moiety L¹ includes a linear backbone of each L¹ comprises a linear backbone of 6 to 20 consecutive atoms, such as 6 to 16 consecutive atoms, such as 8, 9, 10, 11, 12, 13, 14, 15 or 16 consecutive atoms.

In certain embodiments, the linking moiety of formula (XII) includes one or repeating ethylene glycol moieties (e.g., —CH₂CH₂O— or —OCH₂CH₂—). In certain embodiments, the linking moiety of formula (XII) includes 1 to 10 ethylene glycol moieties, such as 1, 2, 3, 4, 5 or 6 ethylene glycol moieties.

In certain embodiments, the linking moiety of formula (XII) includes one or more triazole (e.g., 1,2,3-triazole) containing linking moieties. It is understood that the triazole may be derived from an azido-alkyne click chemistry and thus have two possible orientations depending on the method of synthesis:

In certain embodiments of formula (II′″) n is 2 or more, and L² is selected from:

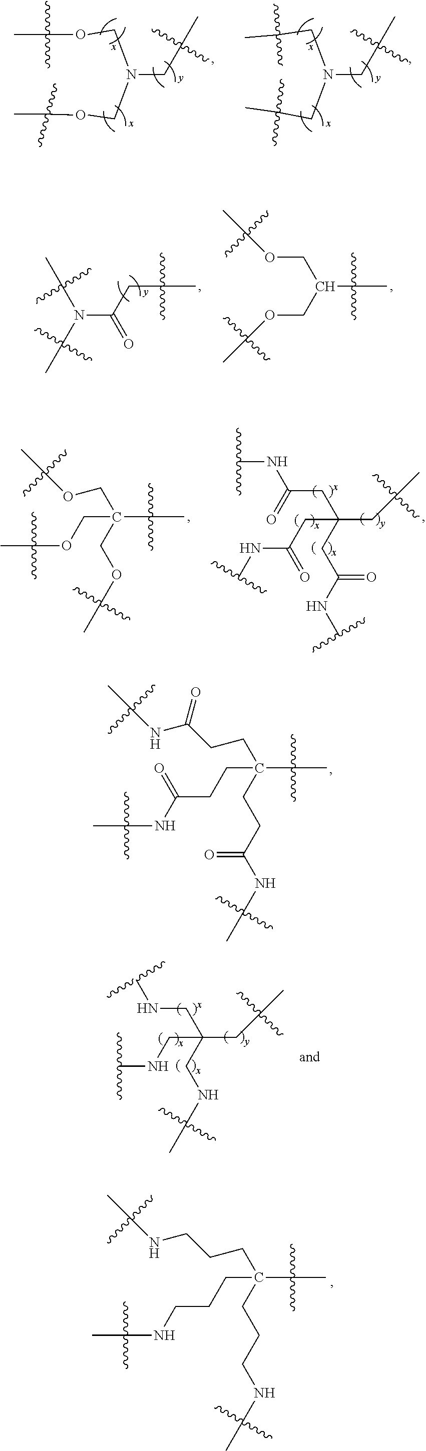

wherein each x and y are independently 1 to 10.

In certain embodiments of formula (II′″) L¹-L² comprises a backbone of 14 or more consecutive atoms between X and the branching atom, such as 14 to 50, 14 to 40, 14 to 35 or 14 to 30 consecutive atoms between X and the branching atom.

In certain embodiments of formula (II′″) or (IIa), L³ comprises a backbone of 10 to 80 consecutive atoms, such as 12 to 70, 12 to 60, or 12 to 50 consecutive atoms. In some embodiments, L comprises of 12 to 70, 12 to 60, 12 to 50, or 10 to 60 consecutive linear or branched chain atoms.

In certain embodiments of formula (II′″) or (IIa), wherein L³ comprises a linking moiety selected from (C₁₀-C₂₀-alkylene (e.g., C₁₂-alkylene), or —(OCH₂CH₂)ₚ—, where p is 1 to 25, such as 3 to 25, 5 to 24, 7 to 25, 10 to 25, 15 to 25 or 20 to 24.

In certain embodiments, L is of formula (IIb):

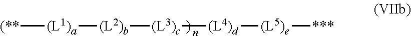

wherein
each L¹ to L⁵ is independently a linking moiety which together provide a linear or branched linker between Z¹ and Y;
a, b, c, d, and e are each independently 0, 1, or 2;
** represents the point of attachment to L¹ of X via Z¹; and
*** represents the point of attachment to Y;
   wherein:
when n is 1, a is 1, and c is 0; and
when n is >1, a is 1, and c is 1.

In some embodiments, L is of formula (IIb′):

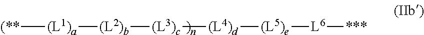

wherein:
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

In certain embodiments of the linker of formula (IIb), L¹ to L⁵ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, - NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non- naturally occurring amino acid residue), —NH—, and —NMe-, wherein each p is independently 1 to 50.

In certain embodiments, L is of formula (IIb′):

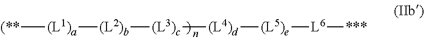

wherein:
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

In certain embodiments, each L¹ to L⁵ independently comprises one or more linking moieties independently selected from —C₁₋₂₀-alkylene-, —NHC(O)—C₁₋₆-alkylene-, —C(O)NH—C₁₋₆-alkylene-, —NH-C ₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50;
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹⁶—, —C₁₋₆-alkylene-NR¹⁶C(S)NR¹⁶—, —O(CH₂)ₚ—, —(OCH₂CH₂)ₚ—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹—; and
each R¹⁶ is independently —H, (C₁-C₆)alkyl, or monocyclic heteroaryl.

In certain embodiments, each L¹ to L⁵ is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C1._-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50; and

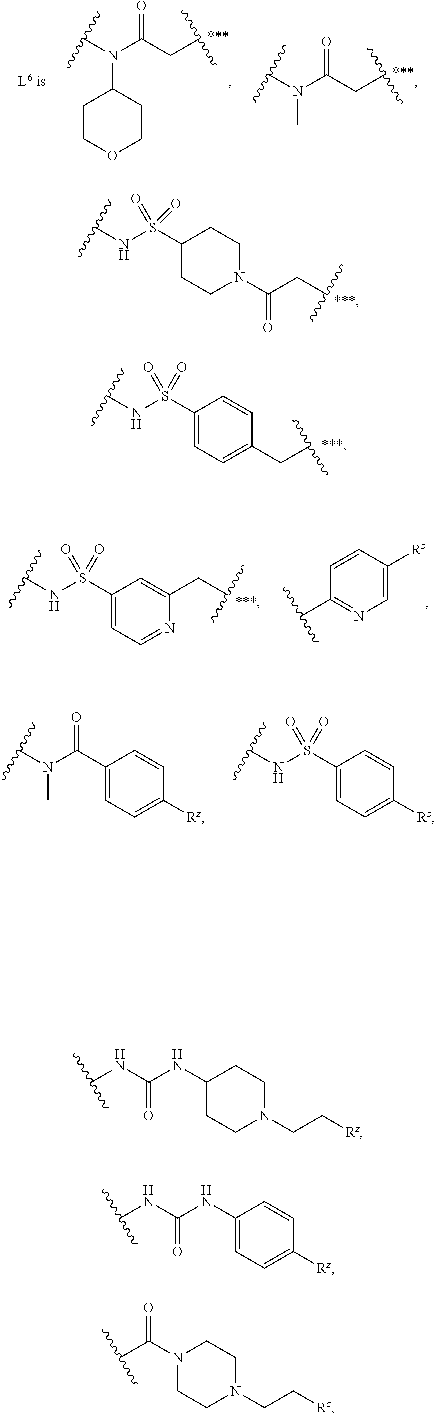

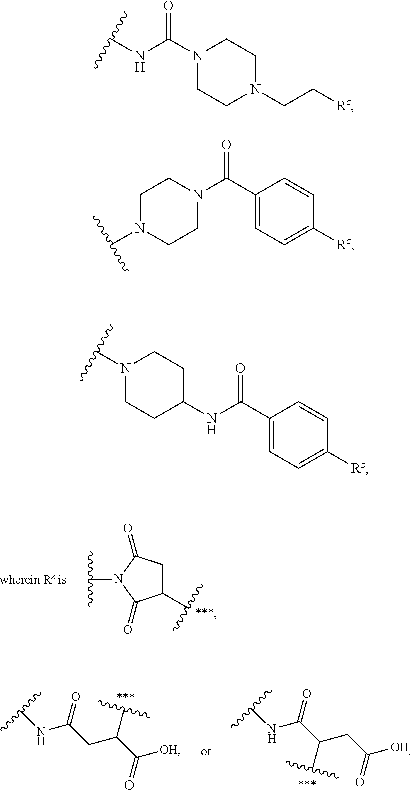

In certain embodiments of formula (IIb), -(L¹)ₐ- comprises an optionally substituted alkyl or ethylene glycol linking moiety. In certain cases, L¹ comprises an optionally substituted —C₁₋₆-alkylene-. In certain cases, L¹ comprises an ethylene glycol linking moiety.

In certain embodiments of formula (IIb), L¹ is independently selected from: —C₁₋₆-alkylene-, —(CH₂CH₂O)₁—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl. In certain cases, L¹ is —C₁₋₆-alkylene-, such as —C₁₋₃-alkylene-. In certain cases, L¹ is —(CH₂CH₂O)ₜ—, where t is 1 to 20, such as 1 to 15, 1 to 10, 1 to 8, 1 to 6, or 1 to 4. In certain cases, L¹ is —C₁₋₆-alkylene-NR⁴CO—. In certain cases, L¹ is —C₁₋₆-alkyleneCONH-. In certain cases, L¹ is or OCH₂.

In some embodiments of formula (IIb), one or more L¹ is independently —CH₂O—; —(CH₂CH₂O)ₜ—, —NR⁴CO—, —C₁₋₆-alkylene-,

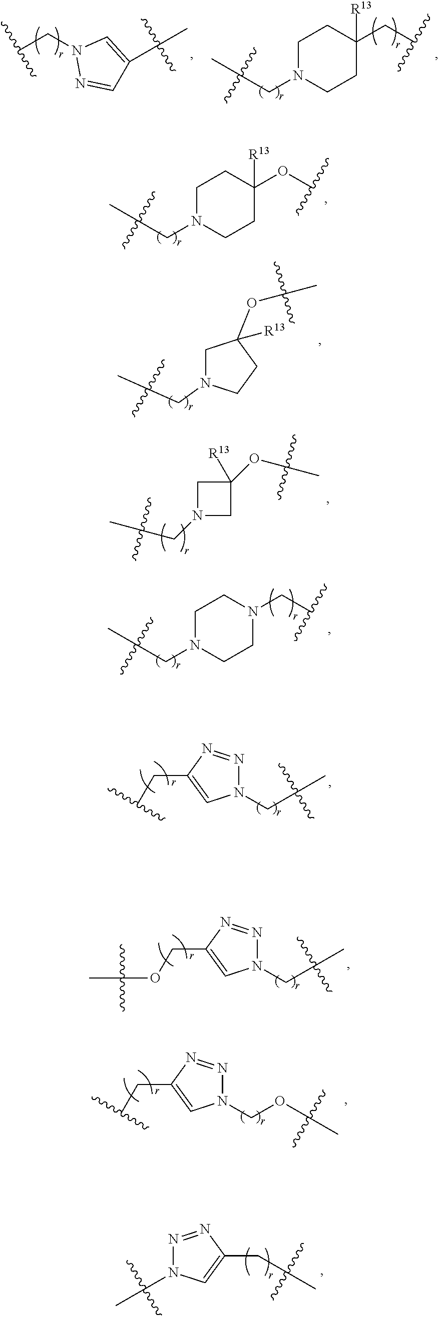

wherein: R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
each r independently 0 to 20, and any of the L¹ moieties are optionally further substituted.

In certain embodiments of formula (IIb), L² is independently selected from: —NR^{4′}CO—C₁₋₆-alkylene-, —CONR^{4′}—C₁₋₆-alkylene,

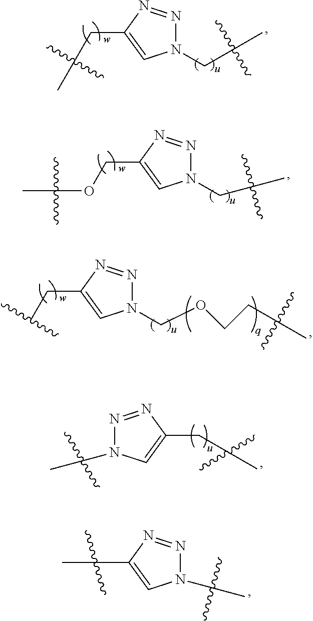

—OCH₂—, and —(OCH₂CH₂)_{q}—, wherein q is 1 to 10, u is 0 to 10, w is 1 to 10, and R^{4′} is independently selected from H, and optionally substituted (C₁-C₆)alkyl. In certain cases, L² is —NR^{4′}CO—C₁₋₆-alkylene-. In certain cases, L² is —CONR^{4′}—C₁₋₆-alkylene.

In certain cases, L² is

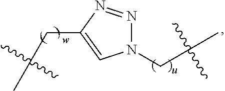

where w is 1 and u is 0 or 1.

In certain cases, L² is

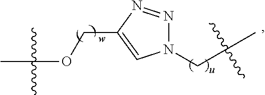

where w is 1 and u is 0 or 1.

In certain cases, L² is

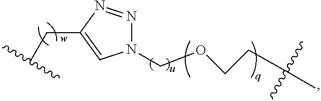

where w is 1, u is 0 or 1, and q is 1.

In certain cases, L² is

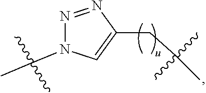

where u is 0 or 1.

In certain cases, L² is

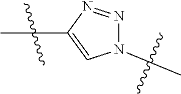 [SMILES: *c1cn(*)nn1]

In certain embodiments, L² is —OCH₂—. In certain other embodiments, L² is (OCH₂CH₂)_{q}—, and q is 1 to 10, 1 to 9, 1 to 8, 1 to 7, 1 to 6, 1 to 5, 1 to 4, 1 to 3 or 1 to 2. In certain cases, q is 2 to 8, such as 2 to 6, 4 to 6, or 2 to 4.

In certain embodiments of formula (IIb), L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂, wherein t is 1 to 20. In certain cases, L⁴ is absent. In certain cases, L⁴ is —C₁₋₆-alkylene-. In certain cases, L⁴ is —(CH₂CH₂O)ₜ—, where t is 1 to 20, such as 1 to 15, 1 to 12, 1 to 10, 1 to 8, 1 to 6, 1 to 4 or 1 to 3. In certain cases, L⁴ is —C₁₋₆-alkylene-NHCO—. In certain cases, L⁴ is —C₁₋₆-alkyleneCONH-. In certain cases, L⁴ is OCH₂.

In some embodiments of the subject compounds, n is 1 and L³ in formula (IIb) is absent.

In certain embodiments of the subject compounds, n is 2 or more, and L³ of formula (IIb) is a branched linking moiety.

Accordingly, in some embodiments of formula (IIb), L³ is a branched linking moiety, e.g., a divalent, or a trivalent linking moiety. For example, an L³ linking moiety can be of the one of the following general formula:

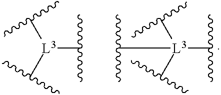

In some embodiments of formula (IIb), the branched linking moiety can be of higher valency and be described by one of the one of the following general formula:

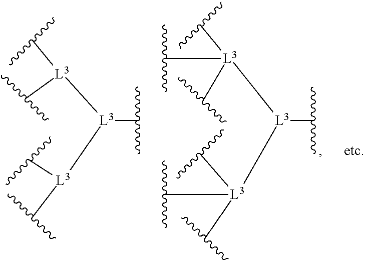

where any two L³ groups can be directed linked or connected via optional linear linking moieties (e.g., as described herein).

In some embodiments of formula (IIb), the branched linking moiety can include one, two or more L³ linking moieties, each being trivalent moieties, which when linked together can provide for multiple branching points for covalent attachment of the ligands and be described by the following general formula:

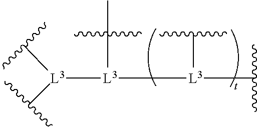

where t is 0 to 500, such as 0 to 100, 0 to 20, or 0 to 10.

In some embodiments, the branched linking moiety (e.g., L³) comprises one or more of an amino acid residue (e.g., Asp, Lys, Orn, Glu), N-substituted amido (—N(−)C(═O)—), tertiary amino, polyol (e.g., O-substituted glycerol), and the like.

In some embodiments of formula (IIb), one or more L³ is a branching moiety selected from

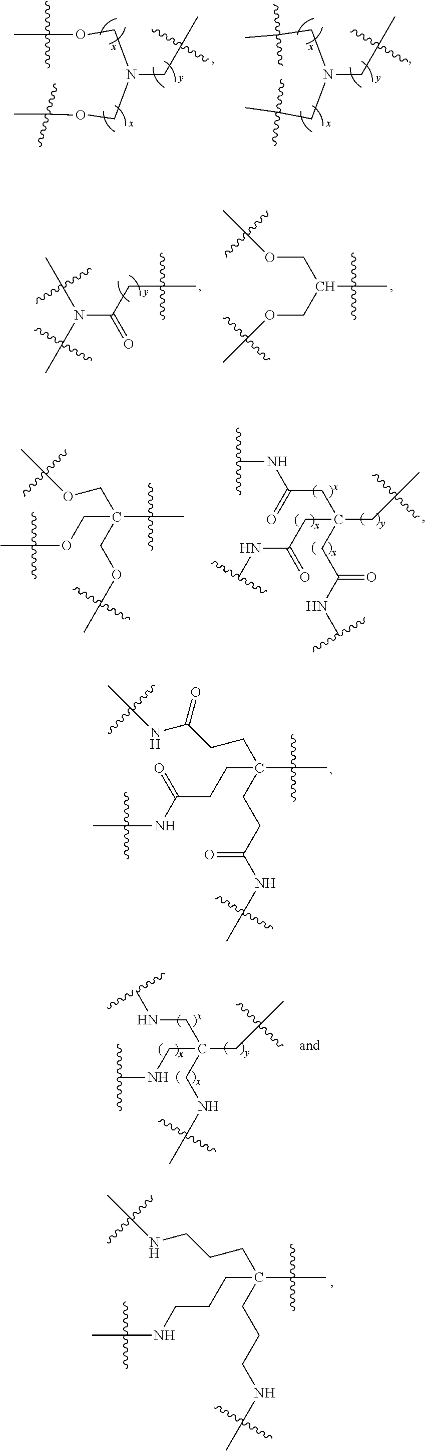

wherein each x and y are each independently 1 to 10, such as 1-6, 1-3, e.g., 1 or 2. In some cases, each x is 1, 2 or 3, e.g., 2.

In some embodiments of formula (IIb), L⁵ is selected from —CH₂O—; —(CH₂CH₂O)ₜ—, —NR⁴CO—, —C₁₋₆-alkylene-,

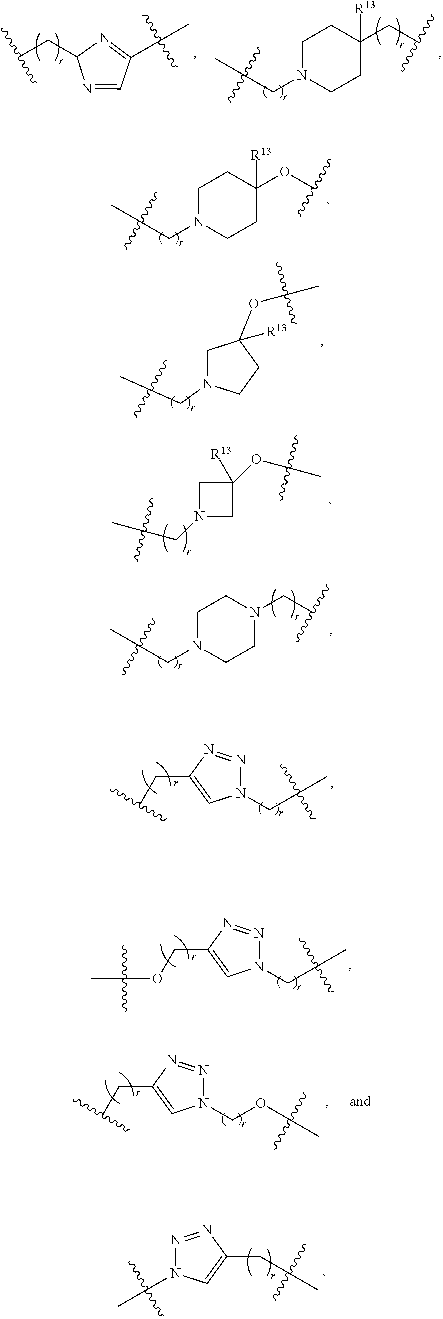

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹; each r independently 0 to 20, and any of the L⁵ moieties are optionally further substituted.

In certain cases, L⁵ is —CH₂O—. In certain cases, L⁵ is —(CH₂CH₂O)ₜ—, where t is 1 to 20, such as 1-15, 1-12, 1-10, 1-8, 1-6, or 1 to 4. In certain cases, L⁵ is —NR⁴CO—, where R⁴ is H, or optionally substituted (C₁-C₆)alkyl. In certain cases, L⁵ is —C₁₋₆-alkylene-.

In certain cases, L⁵ is, where r is

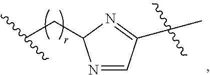

where r is 0 to 20, such as 0 to 15, 0 to 10, 0 to 8, or 0 to 5.

In certain cases, L⁵ is

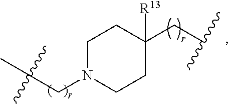

where each r is independently 0 to 20, such as 0 to 15, 0 to 10, 0 to 8, or 0 to 5 and R¹³ is H, or optionally substituted (C₁-C₆)alkyl.

In certain cases, L⁵ is

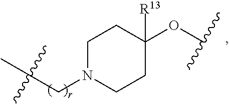

where r is 0 to 20, such as 0 to 15, 0 to 10, 0 to 8, or 0 to 5 and R¹³ is H, or optionally substituted (C₁-C₆)alkyl.

In certain cases, L⁵ is

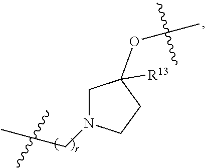

where r is 0 to 20, such as 0 to 15, 0 to 10, 0 to 8, or 0 to 5, and R¹³ is H, or optionally substituted (C₁-C₆)alkyl.

In certain cases, L is

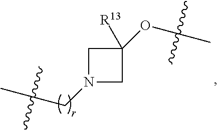

where r is 0 to 20, such as 0 to 15, 0 to 10, 0 to 8, or 0 to 5, and R¹³ is H, or optionally substituted (C₁-C₆)alkyl.

In certain cases, L⁵ is

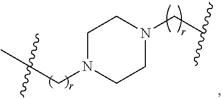

where each r is independently 0 to 20, such as 0 to 15, 0 to 10, 0 to 8, or 0 to 5.

In certain cases, L⁵ is

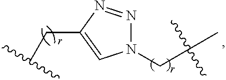

where each r is independently 0 to 20, such as 0 to 15, 0 to 10, 0 to 8, or 0 to 5.

In certain cases, L⁵ is

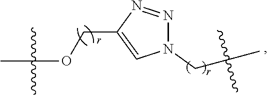

where each r is independently 0 to 20, such as 0 to 15, 0 to 10, 0 to 8, or 0 to 5.

In certain cases, L⁵ is

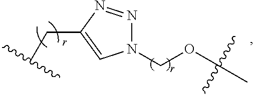

where each r is independently 0 to 20, such as 0 to 15, 0 to 10, 0 to 8, or 0 to 5.

In certain cases, L⁵ is

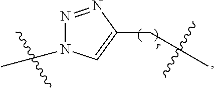

where r is 0 to 20, such as 0 to 15, 0 to 10, 0 to 8, or 0 to 5.

In some embodiments of formula (IIb), L⁵ comprises one or more of: an amino acid residue (e.g., Asp, Lys, Orn, Glu), an amino acid analogue, N-substituted amido (—N(—)C(═O)—), tertiary amino, polyol (e.g., 0-substituted glycerol), and the like. Analogs of an amino acid, include but not limited to, unnatural amino acids, as well as other modifications known in the art. The amino acid includes L-amino acids, D-amino acids, or both, and may contain any of a variety of amino acid modifications or analogs known in the art.

In some embodiments of formula (IIb), L¹-L⁵ comprises one or more of the following units:

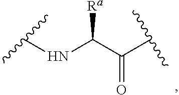

where R^{a} is (C₁-C₆)alkyl or substituted (C₁-C₆)alkyl, e.g., a (C₁-C₆)alkyl optionally substituted with amine, a tertiary amine, optionally substituted alkoxy, optionally substituted carboxyl, optionally substituted aryl, or optionally substituted heteroaryl. It is understood that R^{a} can be linked to a M6PR binding moiety.

In some embodiments, the linker includes a polypeptide scaffold where some or all of the sidechain groups of the amino acid residues have been modified to attach a X binding moiety (e.g., as described herein). It is understood that X binding moieties (e.g., as described herein) can be conjugated to amino acid residues, such as Asp, Lys, Om, Glu, and Ser, of a polypeptide containing linker via a convenient conjugation chemistry. In some embodiments, the linker contains a polylysine polypeptide. In some embodiments, the linker contains a polyornithine polypeptide. In some embodiments, the linker contains a polyserine polypeptide. In some embodiments, the linker contains a polyaspartate polypeptide. The polypeptide can be a randomly polymerized polymer having an average length, or a polymer of defined length prepared e.g., in a controlled stepwise fashion. In some cases, the polypeptide linker segment has a length of 10-100 amino acid residues, such as 20-90, or 20-50 amino acid residues. In some embodiments, the N-terminal or C-terminal of the polypeptide linker segment is modified to include a linking unit to an additional M6PR binding moiety (e.g., as described herein). In some embodiments, the N-terminal or C-terminal of the polypeptide linker segment is modified with one or more linking units (e.g., as described herein) suitable for attachment to a Y moiety of interest.

In certain embodiments of formula (IIb), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In certain embodiments of formula (II), (IIa) or (IIb), the linker comprises 20 to 100 consecutive atoms, such as 20 to 90, 20 to 80, 20 to 70, 20 to 60, 20 to 50, 20 to 40 or 20 to 30 consecutive atoms. In certain cases, the linker comprises 25 to 100 consecutive atoms, such as 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive atoms.

In certain embodiments of formula (II), (IIa) or (IIb), the linker comprises 25 or more consecutive atoms, such as 26 or more, 27 or more, 28 or more, 29 or more or 30 or more consecutive atoms. In certain embodiments of formula (II), (IIa) or (IIb), the linker comprises 30 or more consecutive atoms, such as 31 or more, 32 or more, 33 or more, 34 or more, 35 or more, 36 or more, 37, or more, 38 or more, 39 or more, 40 or even more consecutive atoms.

It is contemplated that compounds of this disclosure having a particular configuration with a linker of desired valency and length can specifically bind with high affinity to both the receptor and a target simultaneously, and exhibit high uptake activity of a target. The conjugates of this disclosure can thus provide for sequestering of a target protein in the cell's lysosome and degrading of the target protein.

### Exemplary Linkers and Linking Moieties

Exemplary linkers and linking moieties that can be utilized in the preparation of compounds of this disclosure are shown in Tables 6-8.

In certain embodiments, the linker is a linear linker or linking moiety as shown in Table 6.

TABLE 6
Exemplary linear linkers and linking moieties

| Linker | |
|---|---|
| No. | Linker structure |
| L1 | 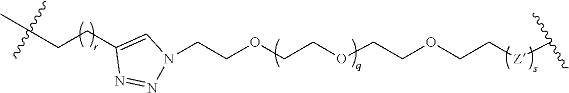 |
| | r is 0 to 10, q is 0 to 20, s is 0 or 1, Z′ is CO, NHCO, CONH or NH |
| L1.1 | 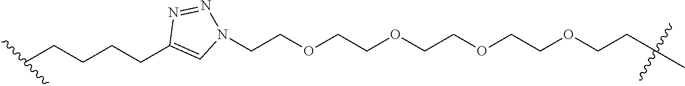 [SMILES: *CCCCc1cn(CCOCCOCCOCCOCC*)nn1] |
| L1.2 | 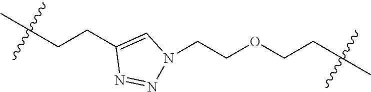 [SMILES: *CCOCCn1cc(CC*)nn1] |
| L1.3 | 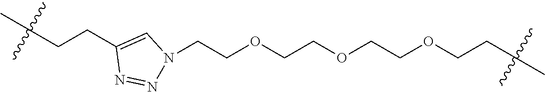 [SMILES: *CCOCCOCCOCCn1cc(CC*)nn1] |
| L1.4 | 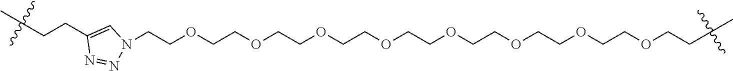 [SMILES: *CCOCCOCCOCCOCCOCCOCCOCCOCCn1cc(CC*)nn1] |
| L1.5 | 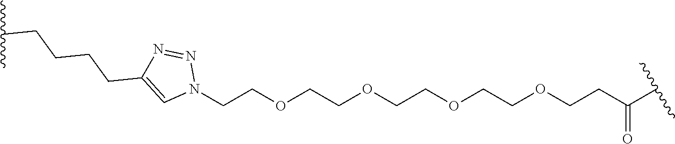 [SMILES: *CCCCc1cn(CCOCCOCCOCCOCCC(*)=O)nn1] |
| L1.6 | 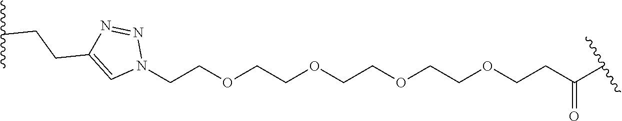 [SMILES: *CCc1cn(CCOCCOCCOCCOCCC(*)=O)nn1] |
| L1.7 | 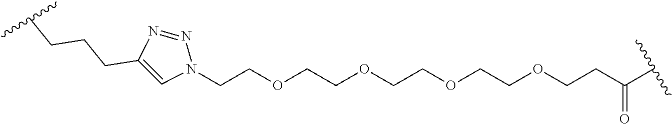 [SMILES: *CCCc1cn(CCOCCOCCOCCOCCC(*)=O)nn1] |
| L1.8 | 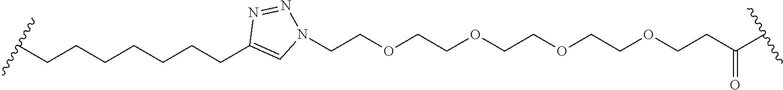 [SMILES: *CCCCCCCc1cn(CCOCCOCCOCCOCCC(*)=O)nn1] |
| L1.9 | 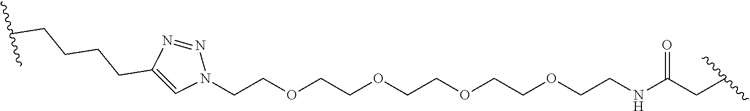 [SMILES: *CCCCc1cn(CCOCCOCCOCCOCCNC(=O)C*)nn1] |
| L1.10 | 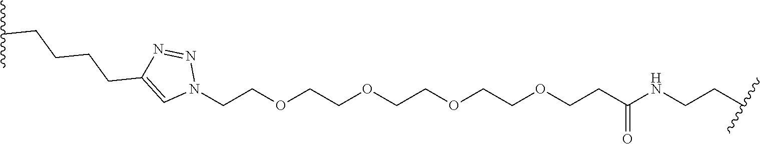 [SMILES: *CCCCc1cn(CCOCCOCCOCCOCCC(=O)NCC*)nn1] |
| L1.11 | 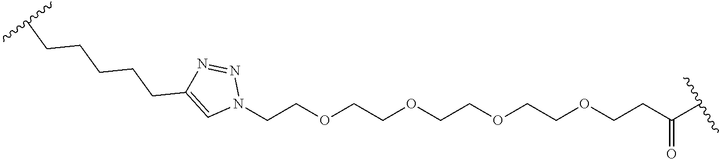 [SMILES: *CCCCCc1cn(CCOCCOCCOCCOCCC(*)=O)nn1] |
| L2 | 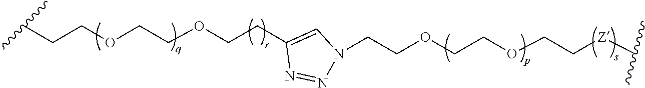 |
| | r is 0 to 10, p and q are 0 to 20, s is 0 or 1, Z′ is CO, NHCO, CONH or NH |
| L2.1 | 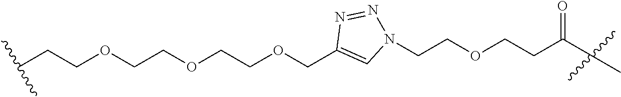 [SMILES: *CCOCCOCCOCc1cn(CCOCCC(*)=O)nn1] |
| L3 | 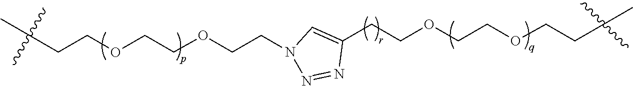 |
| | r is 0 to 10, p and q are independently 0 to 20 |
| L4 | 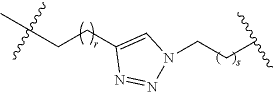 |
| | r is 0 to 10, s is 1 to 10 |
| L5 | 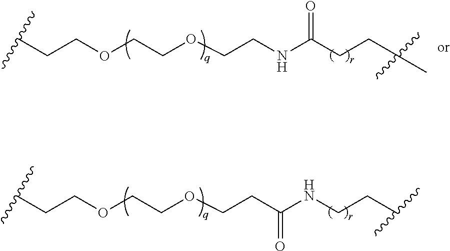 |
| | where r is 0 to 10, q is 0 to 20 |
| L5.1 | 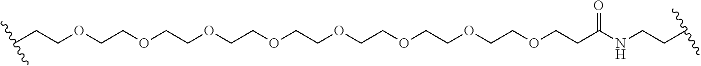 [SMILES: *CCNC(=O)CCOCCOCCOCCOCCOCCOCCOCCOCC*] |
| L6 | 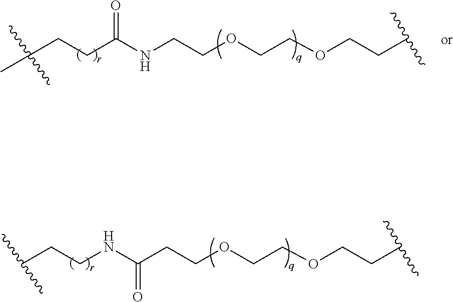 |
| | r is 0 to 10, q is 0 to 20 |
| L7 | 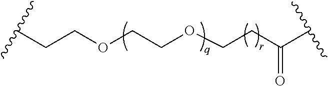 |
| | r is 0 to 10, q is 0 to 20 |
| L7.1 | 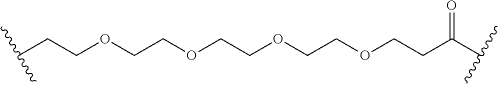 [SMILES: *CCOCCOCCOCCOCCC(*)=O] |
| L7.2 | 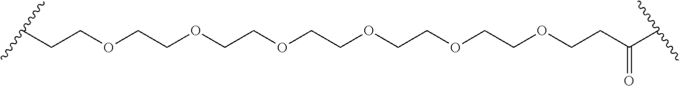 [SMILES: *CCOCCOCCOCCOCCOCCOCCC(*)=O] |
| L7.3 | 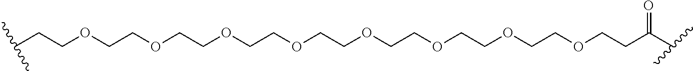 [SMILES: *CCOCCOCCOCCOCCOCCOCCOCCOCCC(*)=O] |
| L8 | 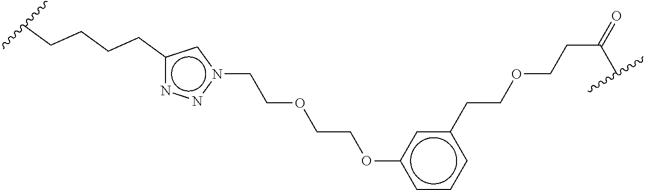 |
| L9 | 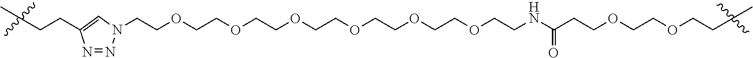 [SMILES: *CCOCCOCCC(=O)NCCOCCOCCOCCOCCOCCOCCn1cc(CC*)nn1] |
| L10 | 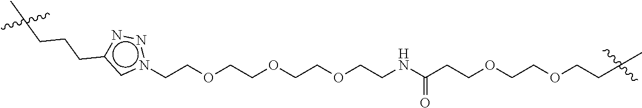 |
| L11 | 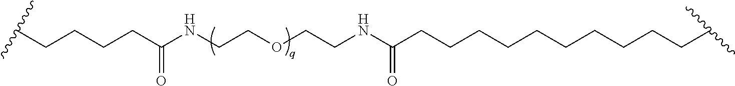 |
| | q is 0 to 10 |
| L12 | 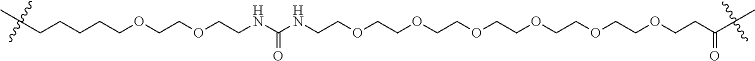 [SMILES: *CCCCCOCCOCCNC(=O)NCCOCCOCCOCCOCCOCCOCCC(*)=O] |
| L13 | 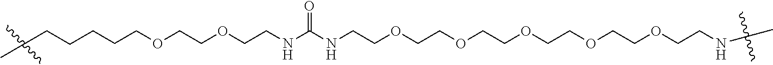 [SMILES: *CCCCCOCCOCCNC(=O)NCCOCCOCCOCCOCCOCCN*] |

Table 7 includes various linker component synthetic precursors (e.g., linear and branched linker precursors) that can be utilized in the preparation of the subject compounds.

TABLE 7
Linker component synthetic precursors

| Re- | |
|---|---|
| agent | |
| # | Structure |
| LC1 | 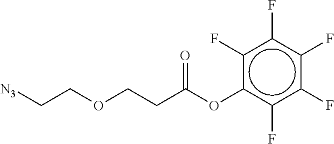 |
| LC1.1 | 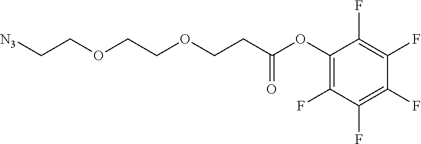 [SMILES: [N-]=[N+]=NCCOCCOCCC(=O)Oc1c(F)c(F)c(F)c(F)c1F] |
| LC2 | 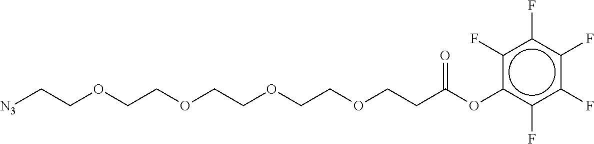 |
| LC3 | 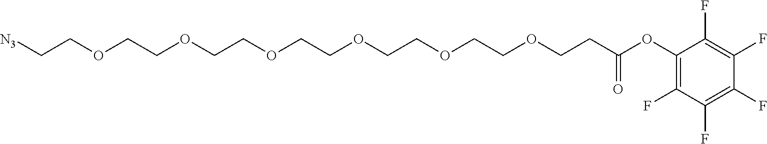 [SMILES: [N-]=[N+]=NCCOCCOCCOCCOCCOCCOCCC(=O)Oc1c(F)c(F)c(F)c(F)c1F] |
| LC3.1 | 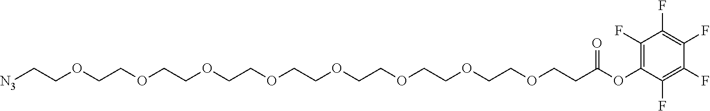 [SMILES: [N-]=[N+]=NCCOCCOCCOCCOCCOCCOCCOCCOCCC(=O)Oc1c(F)c(F)c(F)c(F)c1F] |
| LC3.2 | 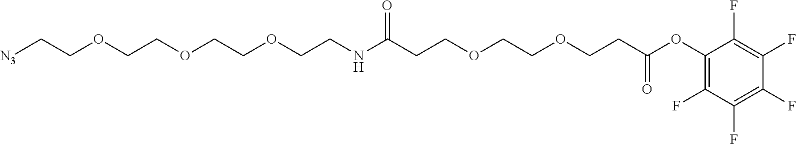 [SMILES: [N-]=[N+]=NCCOCCOCCOCCNC(=O)CCOCCOCCC(=O)Oc1c(F)c(F)c(F)c(F)c1F] |
| LC4 | 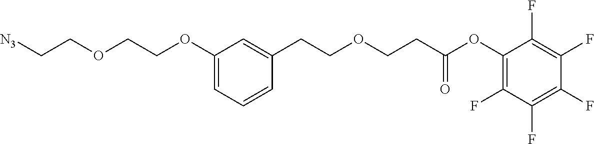 [SMILES: [N-]=[N+]=NCCOCCOc1cccc(CCOCCC(=O)Oc2c(F)c(F)c(F)c(F)c2F)c1] |
| LC5 | 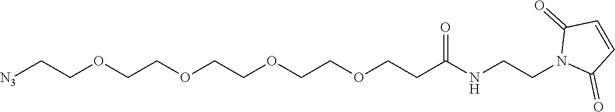 [SMILES: [N-]=[N+]=NCCOCCOCCOCCOCCC(=O)NCCN1C(=O)C=CC1=O] |
| LC6 | 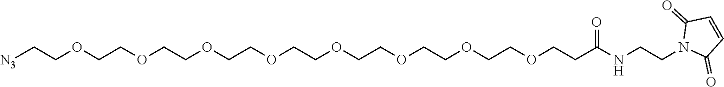 [SMILES: [N-]=[N+]=NCCOCCOCCOCCOCCOCCOCCOCCOCCC(=O)NCCN1C(=O)C=CC1=O] |
| LC7 | 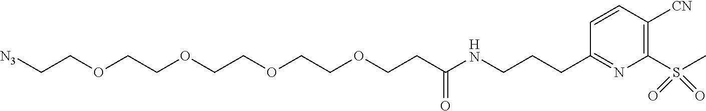 [SMILES: CS(=O)(=O)c1nc(CCCNC(=O)CCOCCOCCOCCOCCN=[N+]=[N-])ccc1C#N] |
| LC8 | 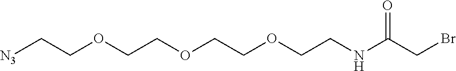 [SMILES: [N-]=[N+]=NCCOCCOCCOCCNC(=O)CBr] |
| LC9 | 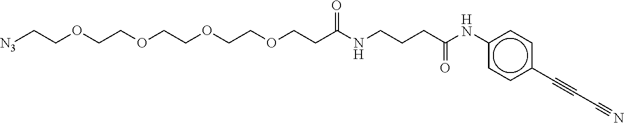 |
| LC9.1 | 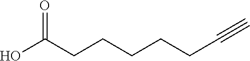 |
| LC10 | 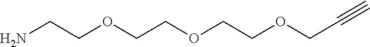 |
| LC10.1 | 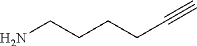 |
| LC10.2 | 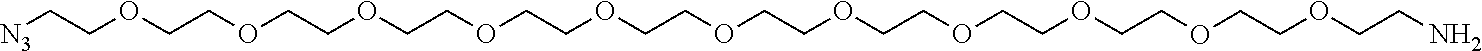 [SMILES: [N-]=[N+]=NCCOCCOCCOCCOCCOCCOCCOCCOCCOCCOCCOCCN] |
| LC10.3 | 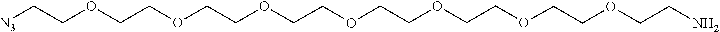 [SMILES: [N-]=[N+]=NCCOCCOCCOCCOCCOCCOCCOCCN] |
| LC10.4 | 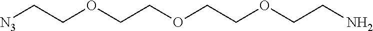 [SMILES: [N-]=[N+]=NCCOCCOCCOCCN] |
| LC10.5 | 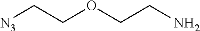 |
| LC10.6 | 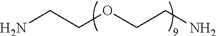 |
| LC10.7 | 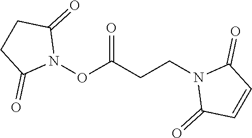 [SMILES: O=C(CCN1C(=O)C=CC1=O)ON1C(=O)CCC1=O] |
| LC10.8 | 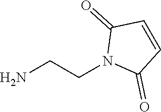 |
| LC11 | 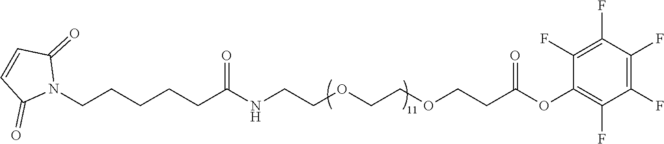 |
| LC12 | 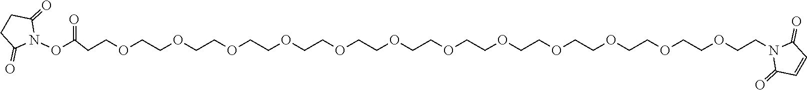 [SMILES: O=C(CCOCCOCCOCCOCCOCCOCCOCCOCCOCCOCCOCCOCCN1C(=O)C=CC1=O)ON1C(=O)CCC1=O] |
| LC13 | 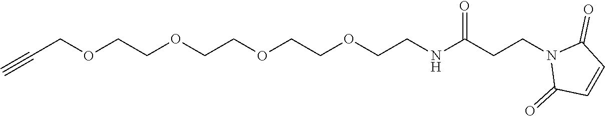 [SMILES: C#CCOCCOCCOCCOCCNC(=O)CCN1C(=O)C=CC1=O] |
| LC14 | 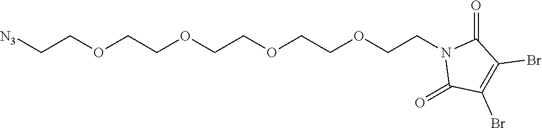 [SMILES: [N-]=[N+]=NCCOCCOCCOCCOCCN1C(=O)C(Br)=C(Br)C1=O] |
| LC15 | 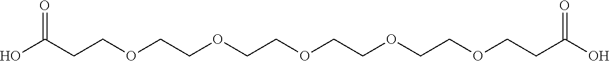 [SMILES: O=C(O)CCOCCOCCOCCOCCOCCC(=O)O] |
| LC16 | 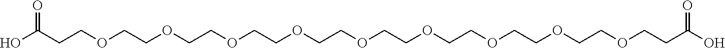 [SMILES: O=C(O)CCOCCOCCOCCOCCOCCOCCOCCOCCOCCC(=O)O] |
| LC17 (1-3) | 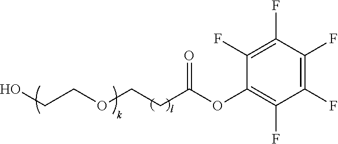 |
| | k = 4, l = 0 |
| | k = 0, l = 12 |
| | k = 2, l = 6 |
| LC18 | 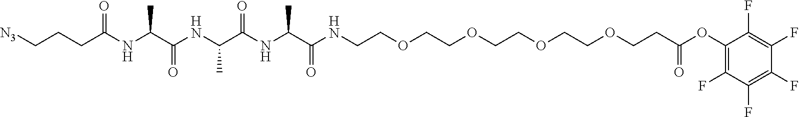 [SMILES: C[C@H](NC(=O)CCCN=[N+]=[N-])C(=O)N[C@@H](C)C(=O)N[C@@H](C)C(=O)NCCOCCOCCOCCOCCC(=O)Oc1c(F)c(F)c(F)c(F)c1F] |
| LC19 | 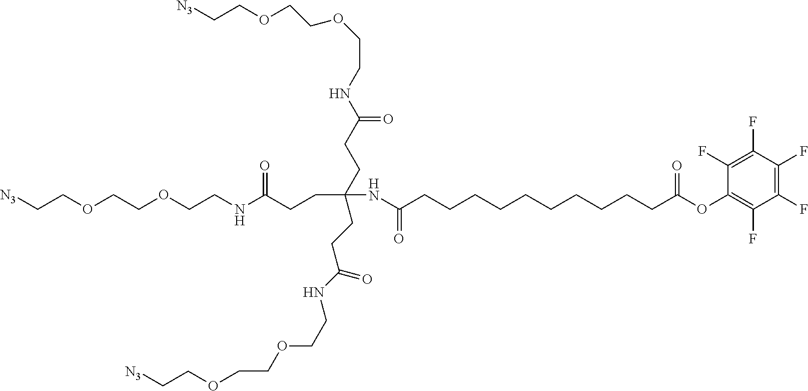 [SMILES: [N-]=[N+]=NCCOCCOCCNC(=O)CCC(CCC(=O)NCCOCCOCCN=[N+]=[N-])(CCC(=O)NCCOCCOCCN=[N+]=[N-])NC(=O)CCCCCCCCCCC(=O)Oc1c(F)c(F)c(F)c(F)c1F] |
| LC19.1 | 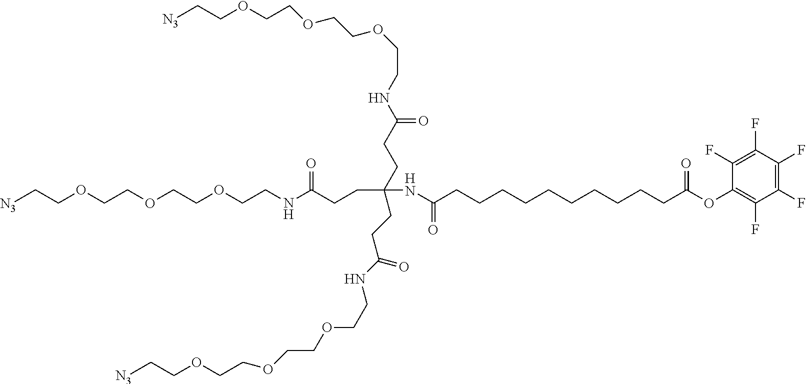 [SMILES: [N-]=[N+]=NCCOCCOCCOCCNC(=O)CCC(CCC(=O)NCCOCCOCCOCCN=[N+]=[N-])(CCC(=O)NCCOCCOCCOCCN=[N+]=[N-])NC(=O)CCCCCCCCCCC(=O)Oc1c(F)c(F)c(F)c(F)c1F] |
| LC20 | 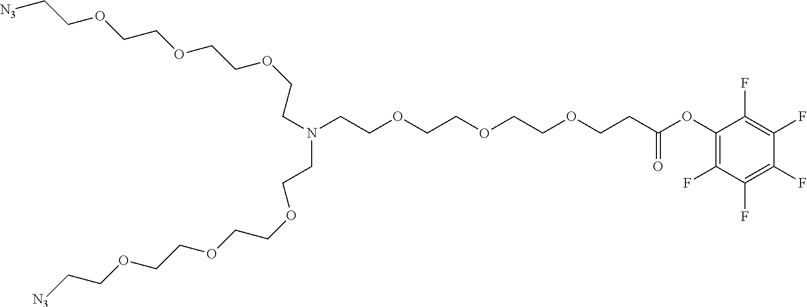 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F] |
| LC21 | 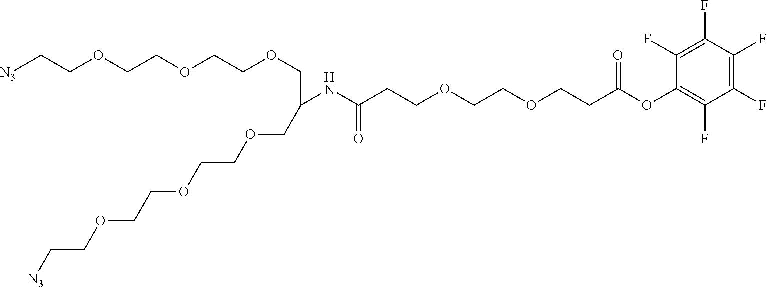 [SMILES: [N-]=[N+]=NCCOCCOCCOCC(COCCOCCOCCN=[N+]=[N-])NC(=O)CCOCCOCCC(=O)Oc1c(F)c(F)c(F)c(F)c1F] |
| LC22 | 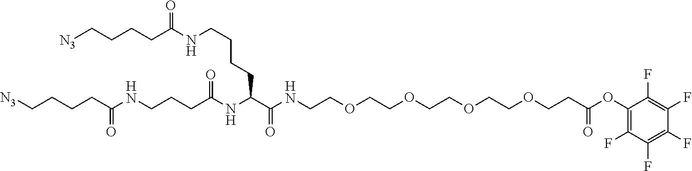 [SMILES: [N-]=[N+]=NCCCCC(=O)NCCCC[C@H](NC(=O)CCCNC(=O)CCCCN=[N+]=[N-])C(=O)NCCOCCOCCOCCOCCC(=O)Oc1c(F)c(F)c(F)c(F)c1F] |
| LC23 | 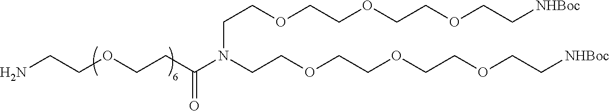 |
| LC24 | 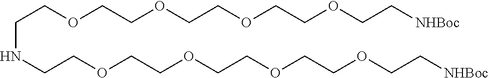 [SMILES: CC(C)(C)OC(=O)NCCOCCOCCOCCOCCNCCOCCOCCOCCOCCNC(=O)OC(C)(C)C] |
| LC25 | 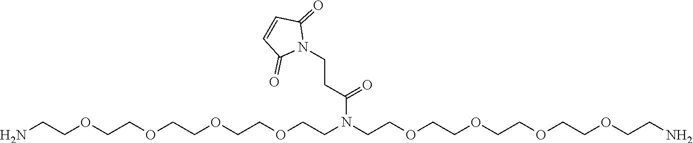 [SMILES: NCCOCCOCCOCCOCCN(CCOCCOCCOCCOCCN)C(=O)CCN1C(=O)C=CC1=O] |
| LC26 | 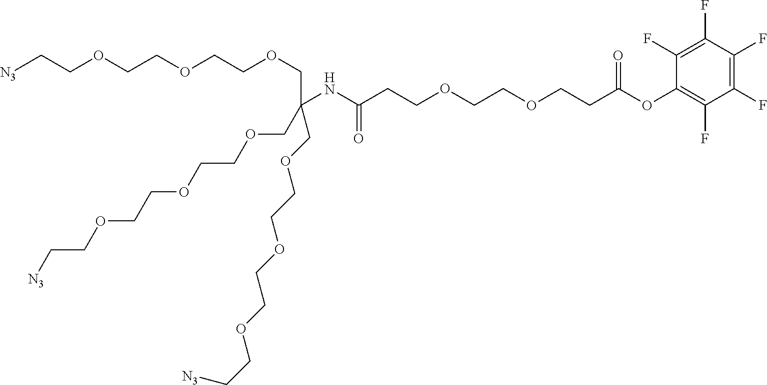 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F] |
| LC27 | 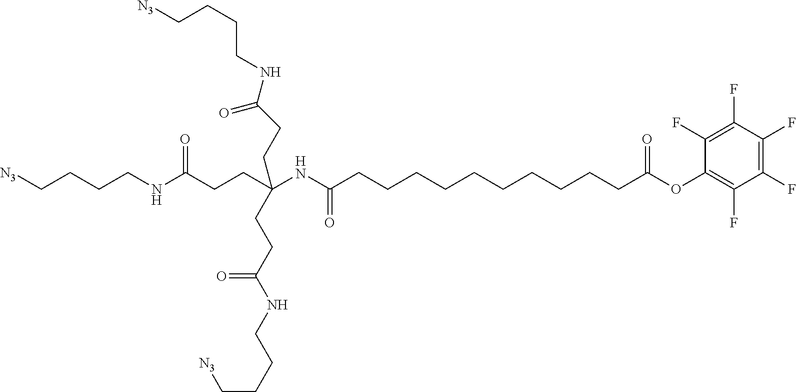 [SMILES: [N-]=[N+]=NCCCCNC(=O)CCC(CCC(=O)NCCCCN=[N+]=[N-])(CCC(=O)NCCCCN=[N+]=[N-])NC(=O)CCCCCCCCCCC(=O)Oc1c(F)c(F)c(F)c(F)c1F] |
| LC28 | 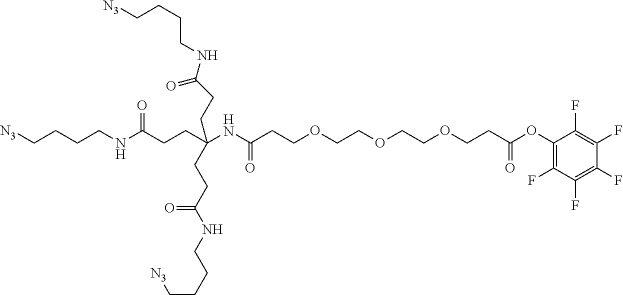 [SMILES: [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)Oc1c(F)c(F)c(F)c(F)c1F] |
| LC29 | 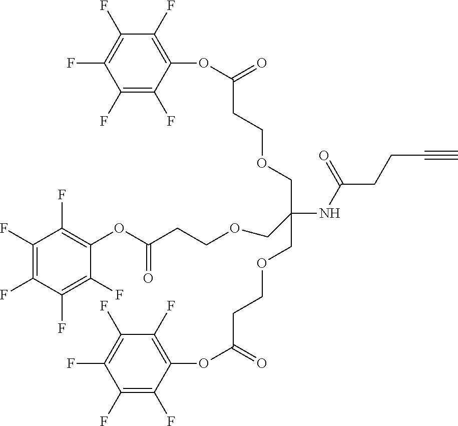 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F] |
| LC30 | 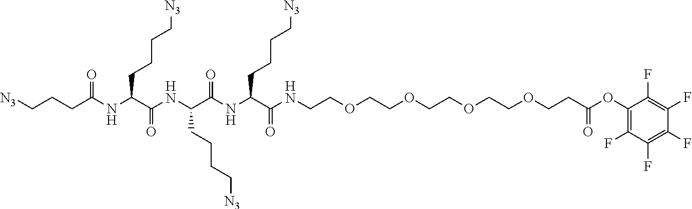 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC31 | 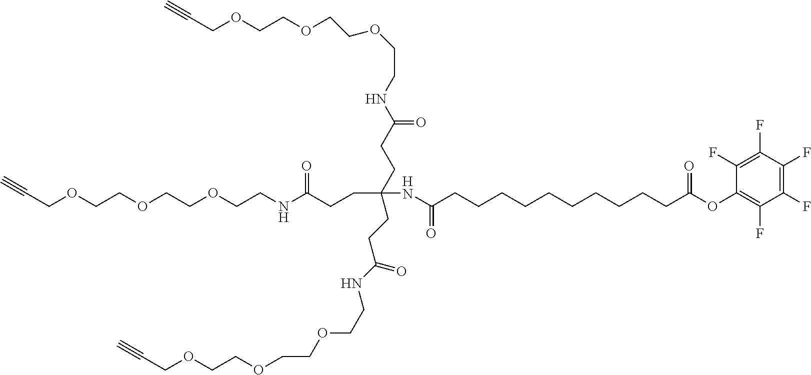 [SMILES: C#CCOCCOCCOCCNC(=O)CCC(CCC(=O)NCCOCCOCCOCC#C)(CCC(=O)NCCOCCOCCOCC#C)NC(=O)CCCCCCCCCCC(=O)Oc1c(F)c(F)c(F)c(F)c1F] |
| LC32 | 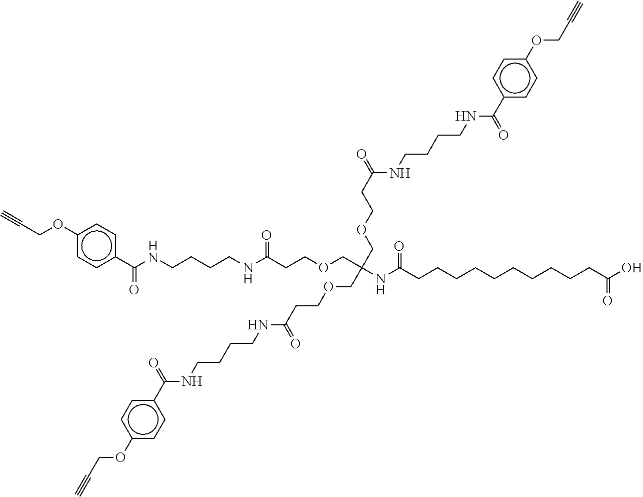 |
| LC33 | 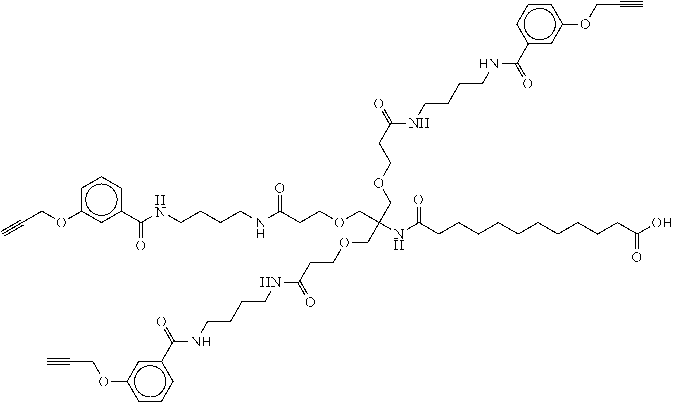 |
| LC34 | 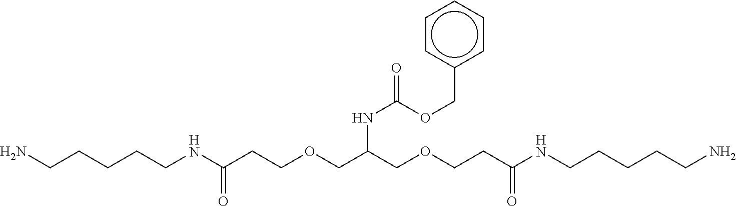 |
| LC35 | 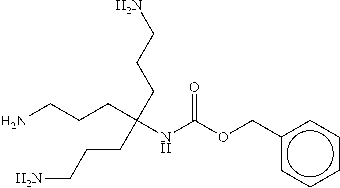 |
| LC36 | 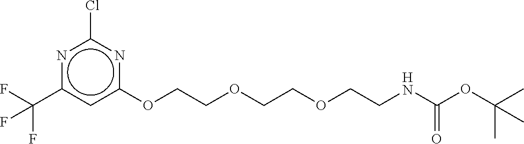 |
| LC37 | 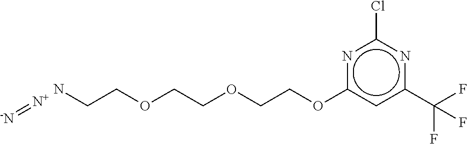 |
| LC38 | 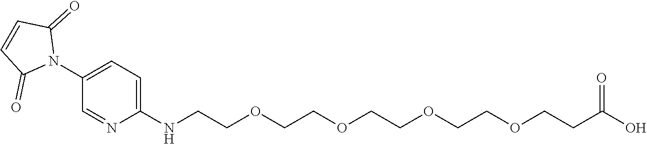 [SMILES: O=C(O)CCOCCOCCOCCOCCNc1ccc(N2C(=O)C=CC2=O)cn1] |
| LC39 | 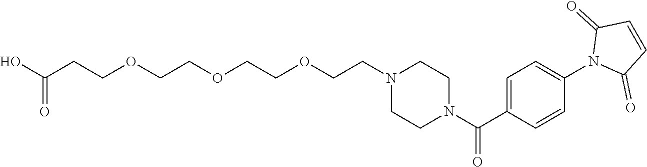 [SMILES: O=C(O)CCOCCOCCOCCN1CCN(C(=O)c2ccc(N3C(=O)C=CC3=O)cc2)CC1] |
| LC40 | 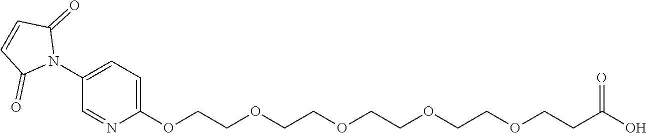 [SMILES: O=C(O)CCOCCOCCOCCOCCOc1ccc(N2C(=O)C=CC2=O)cn1] |
| LC41 | 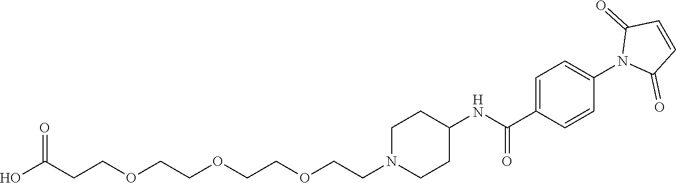 [SMILES: O=C(O)CCOCCOCCOCCN1CCC(NC(=O)c2ccc(N3C(=O)C=CC3=O)cc2)CC1] |
| LC42 | 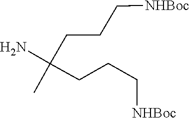 [SMILES: CC(N)(CCCNC(=O)OC(C)(C)C)CCCNC(=O)OC(C)(C)C] |
| LC43 | 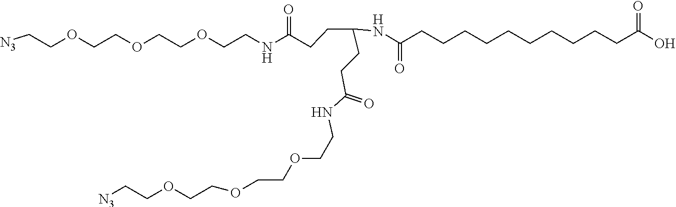 [SMILES: [N-]=[N+]=NCCOCCOCCOCCNC(=O)CCC(CCC(=O)NCCOCCOCCOCCN=[N+]=[N-])NC(=O)CCCCCCCCCCC(=O)O] |
| LC44 | 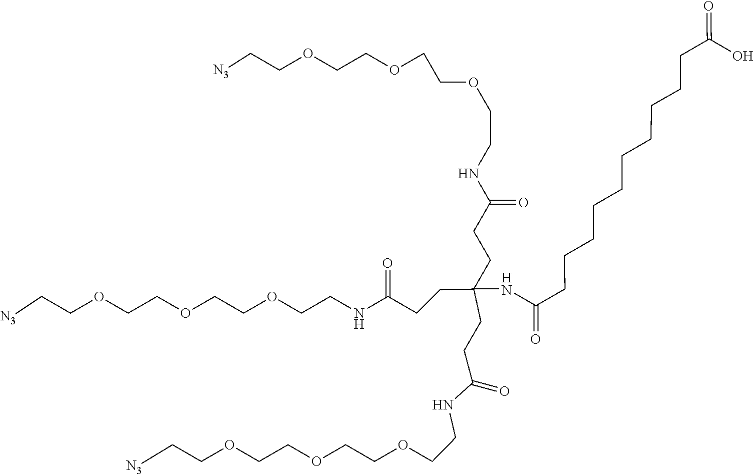 [SMILES: [N-]=[N+]=NCCOCCOCCOCCNC(=O)CCC(CCC(=O)NCCOCCOCCOCCN=[N+]=[N-])(CCC(=O)NCCOCCOCCOCCN=[N+]=[N-])NC(=O)CCCCCCCCCCC(=O)O] |
| LC45 | 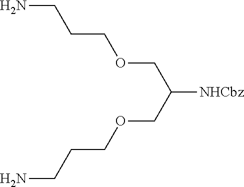 [SMILES: NCCCOCC(COCCCN)NC(=O)OCc1ccccc1] |

In certain embodiments, the linker is a branched linker or linking moiety as shown in Table 8.

TABLE 8
Exemplary branched linkers and branched linking moieties

| Linker | |
|---|---|
| No. | Linker structure |
| L21 | 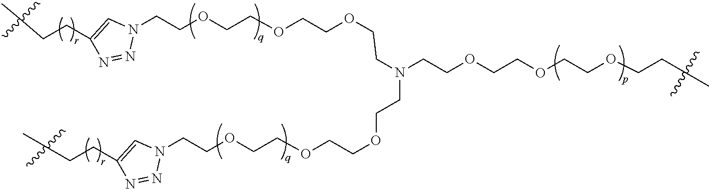 |
| | r is 0 to 10, q and p are independently 0 to 20 |
| L22 | 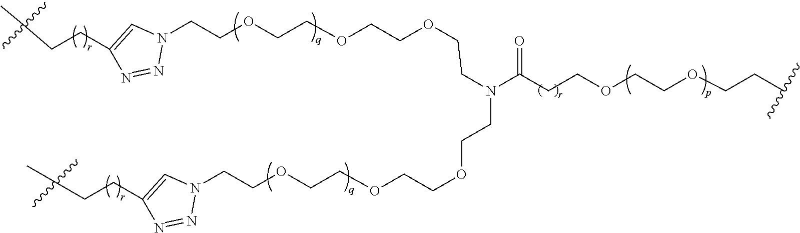 |
| | each r is independently 0 to 10, q and p are independently 0 to 20 |
| L23 | 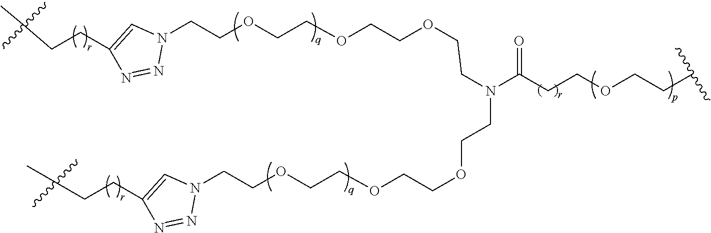 |
| | each r is independently 0 to 10, q and p are independently 0 to 20 |
| L24 | 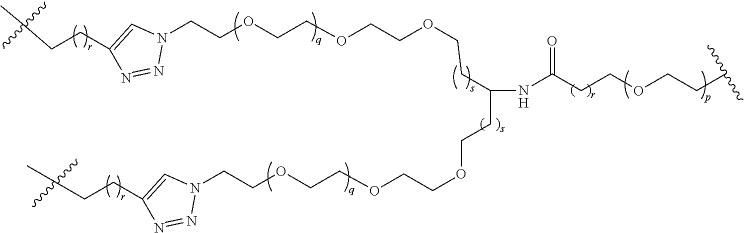 |
| | each r is independently 0 to 10, s is 0 or 1, q and p are independently 0 to 20 |
| L25 | 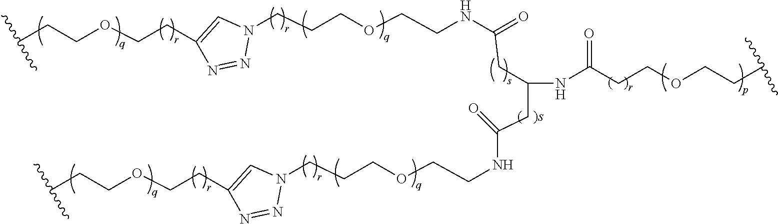 |
| | each r is independently 0 to 10, s is 0 or 1, each q and p is independently 0 to 20 |
| L26 | 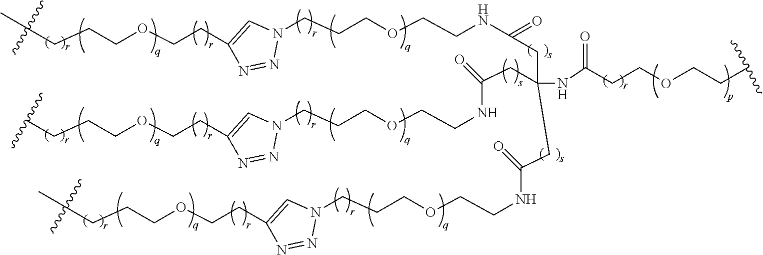 |
| | each r is independently 0 to 10, s is 0 or 1, each q and p is independently 0 to 20 |
| L26.1 | 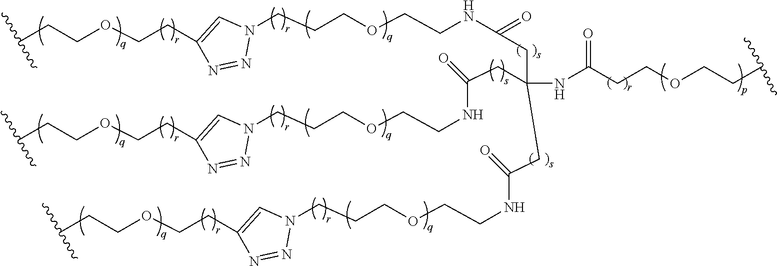 |
| | each r is independently 0 to 10, s is 0 or 1, each q and p is independently 0 to 20 |
| L27 | 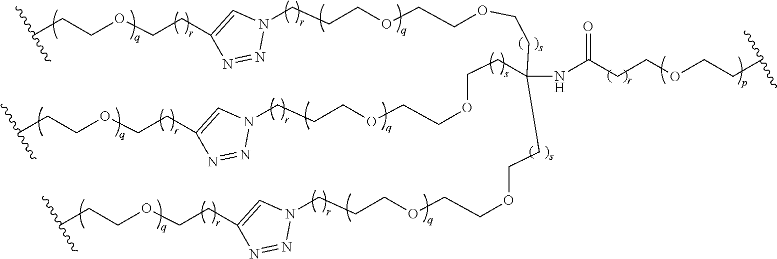 |
| | each r is independently 0 to 10, s is 0 or 1, each q and p is independently 0 to 20 |
| L28 | 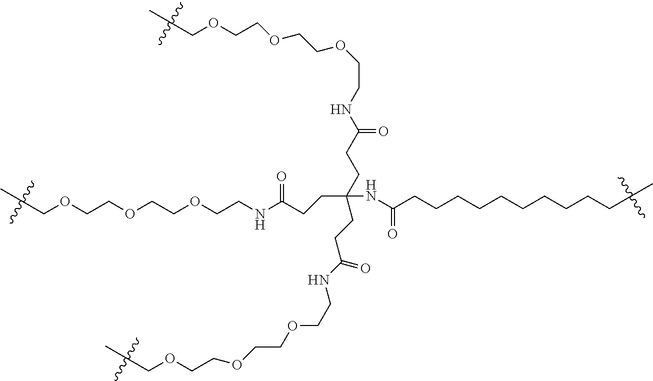 [SMILES: *CCCCCCCCCCC(=O)NC(CCC(=O)NCCOCCOCCOC*)(CCC(=O)NCCOCCOCCOC*)CCC(=O)NCCOCCOCCOC*] |
| L29 | 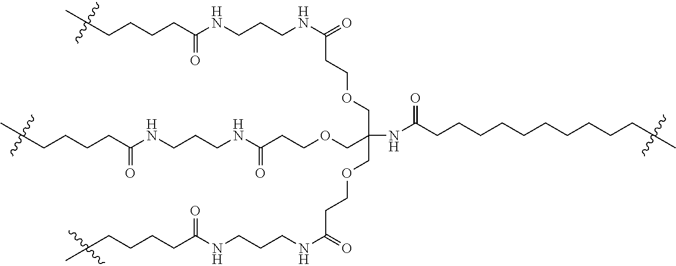 [SMILES: *CCCCCCCCCCC(=O)NC(COCCC(=O)NCCCNC(=O)CCCC*)(COCCC(=O)NCCCNC(=O)CCCC*)COCCC(=O)NCCCNC(=O)CCCC*] |
| L30 | 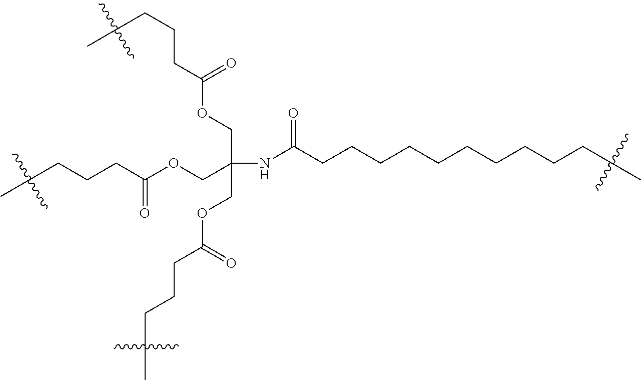 [SMILES: *CCCCCCCCCCC(=O)NC(COC(=O)CCC*)(COC(=O)CCC*)COC(=O)CCC*] |
| L31 | 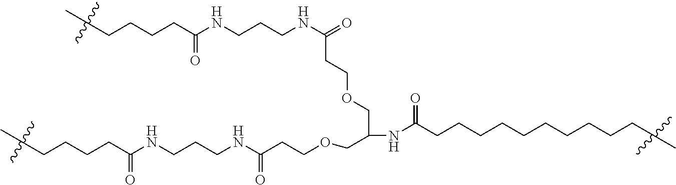 [SMILES: *CCCCCCCCCCC(=O)NC(COCCC(=O)NCCCNC(=O)CCCC*)COCCC(=O)NCCCNC(=O)CCCC*] |
| L32 | 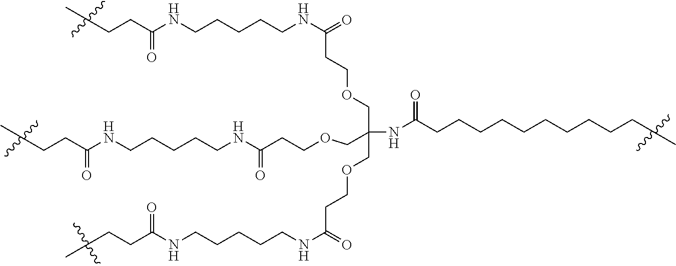 [SMILES: *CCCCCCCCCCC(=O)NC(COCCC(=O)NCCCCCNC(=O)CC*)(COCCC(=O)NCCCCCNC(=O)CC*)COCCC(=O)NCCCCCNC(=O)CC*] |
| L33 | 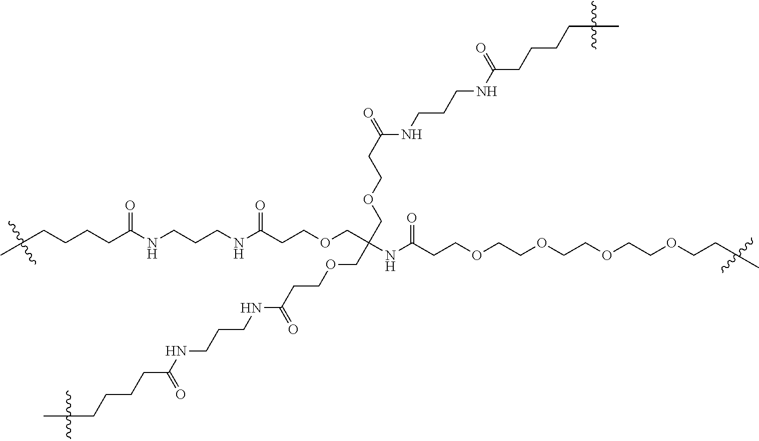 [SMILES: *CCCCC(=O)NCCCNC(=O)CCOCC(COCCC(=O)NCCCNC(=O)CCCC*)(COCCC(=O)NCCCNC(=O)CCCC*)NC(=O)CCOCCOCCOCCOCC*] |
| L34 | 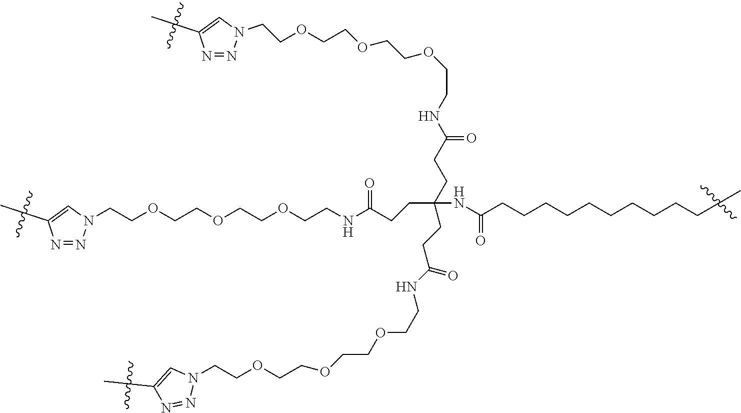 [SMILES: *CCCCCCCCCCC(=O)NC(CCC(=O)NCCOCCOCCOCCn1cc(*)nn1)(CCC(=O)NCCOCCOCCOCCn1cc(*)nn1)CCC(=O)NCCOCCOCCOCCn1cc(*)nn1] |
| L35a L36a | 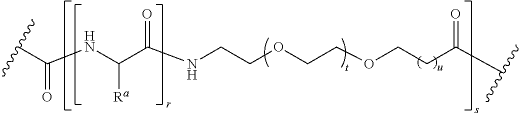 |
| | 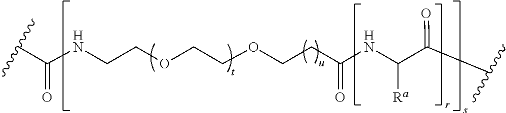 |
| | where r is 1-3, t is 3-5, u is 0 or 1, and s is 2-5 |
| L36 | 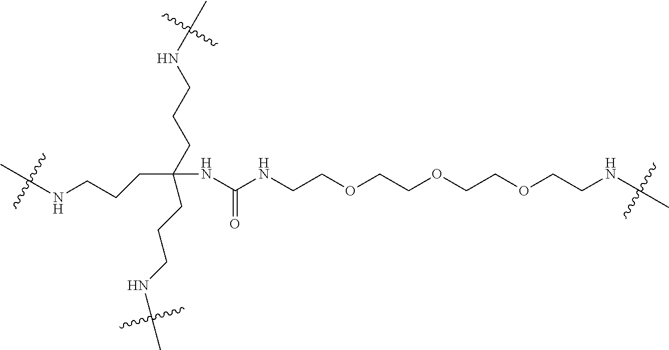 [SMILES: *NCCCC(CCCN*)(CCCN*)NC(=O)NCCOCCOCCOCCN*] |
| L37 | 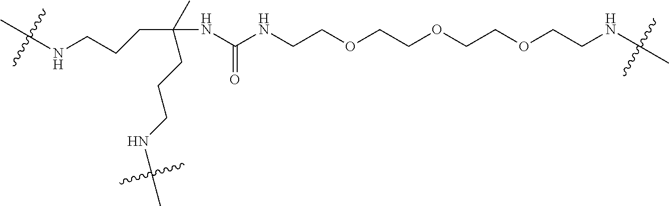 [SMILES: *NCCCC(C)(CCCN*)NC(=O)NCCOCCOCCOCCN*] |
| L38 | 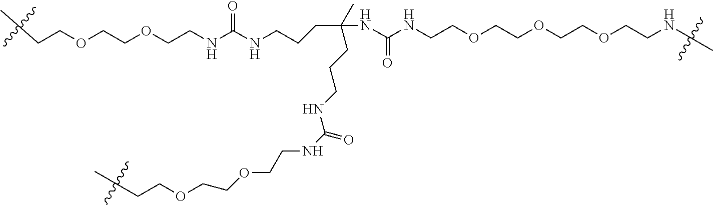 [SMILES: *CCOCCOCCNC(=O)NCCCC(C)(CCCNC(=O)NCCOCCOCC*)NC(=O)NCCOCCOCCOCCN*] |
| L39 | 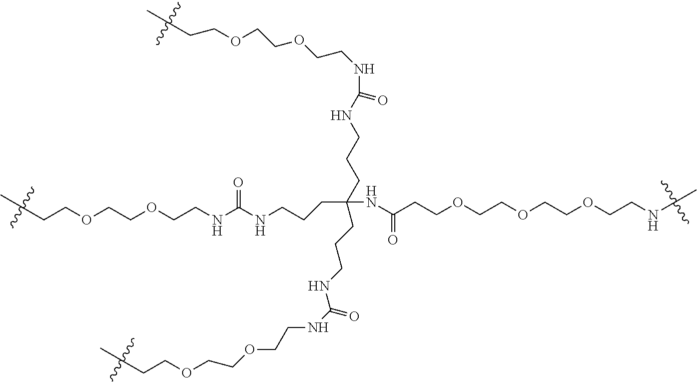 [SMILES: *CCOCCOCCNC(=O)NCCCC(CCCNC(=O)NCCOCCOCC*)(CCCNC(=O)NCCOCCOCC*)NC(=O)CCOCCOCCOCCN*] |
| L40 | 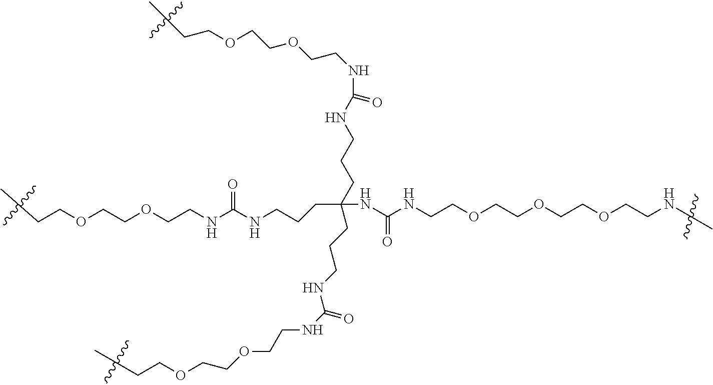 [SMILES: *CCOCCOCCNC(=O)NCCCC(CCCNC(=O)NCCOCCOCC*)(CCCNC(=O)NCCOCCOCC*)NC(=O)NCCOCCOCCOCCN*] |

Table 9 illustrates exemplary synthetic precursors of linker components that are used to prepare compounds of this disclosure, e.g., via a conjugation chemistry. It is understood that a variety of homologs of the structures shown in Table 9 are also encompassed by this disclosure that provide for linkers of a variety of lengths. It is understood that alternative chemoselective ligation groups and other chemical functional groups can also be incorporated as needed to prepare a desired linker.

TABLE 9
Linker component synthetic precursors

| Re- | |
|---|---|
| agent | |
| # | Structure |
| LC1 | 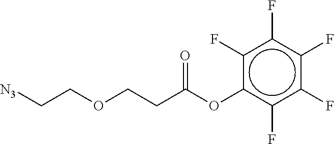 |
| LC2 | 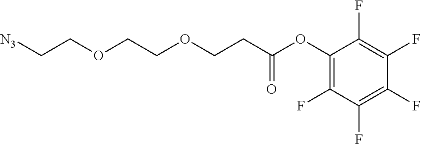 [SMILES: [N-]=[N+]=NCCOCCOCCC(=O)Oc1c(F)c(F)c(F)c(F)c1F] |
| LC3 | 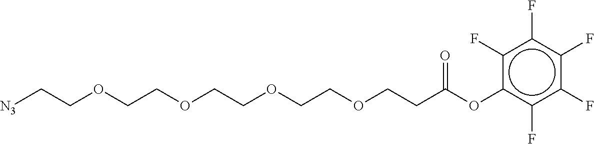 |
| LC4 | 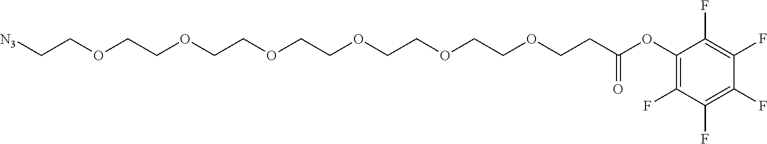 [SMILES: [N-]=[N+]=NCCOCCOCCOCCOCCOCCOCCC(=O)Oc1c(F)c(F)c(F)c(F)c1F] |
| LC5 | 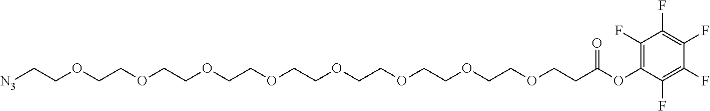 [SMILES: [N-]=[N+]=NCCOCCOCCOCCOCCOCCOCCOCCOCCC(=O)Oc1c(F)c(F)c(F)c(F)c1F] |
| LC6 | 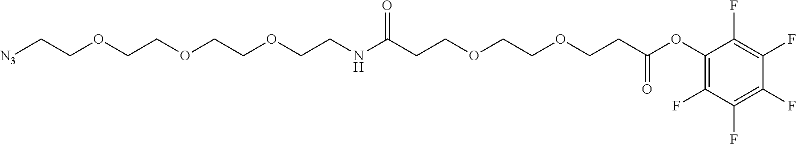 [SMILES: [N-]=[N+]=NCCOCCOCCOCCNC(=O)CCOCCOCCC(=O)Oc1c(F)c(F)c(F)c(F)c1F] |
| LC7 | 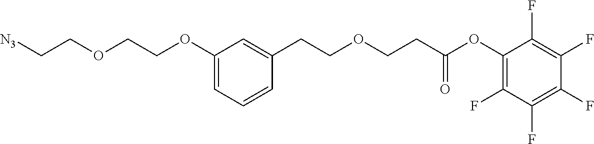 [SMILES: [N-]=[N+]=NCCOCCOc1cccc(CCOCCC(=O)Oc2c(F)c(F)c(F)c(F)c2F)c1] |
| LC8 | 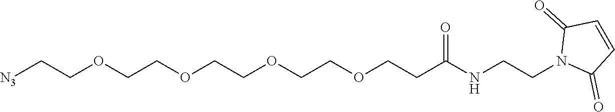 [SMILES: [N-]=[N+]=NCCOCCOCCOCCOCCC(=O)NCCN1C(=O)C=CC1=O] |
| LC9 | 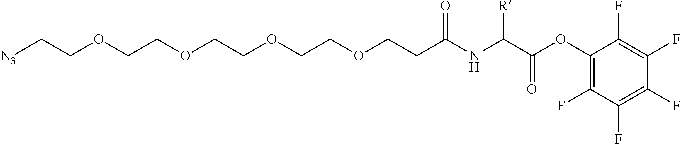 |
| LC10 | 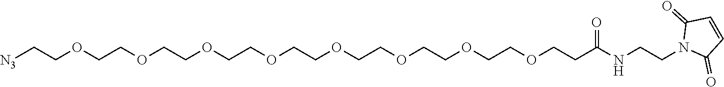 [SMILES: [N-]=[N+]=NCCOCCOCCOCCOCCOCCOCCOCCOCCC(=O)NCCN1C(=O)C=CC1=O] |
| LC11 | 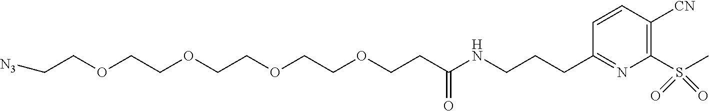 [SMILES: CS(=O)(=O)c1nc(CCCNC(=O)CCOCCOCCOCCOCCN=[N+]=[N-])ccc1C#N] |
| LC12 | 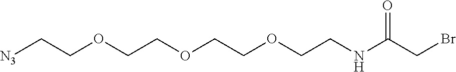 [SMILES: [N-]=[N+]=NCCOCCOCCOCCNC(=O)CBr] |
| LC13 | 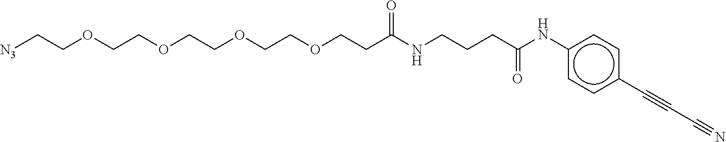 |
| LC14 | 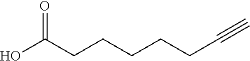 |
| LC15 | 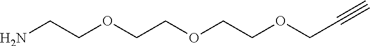 |
| LC16 | 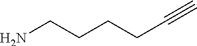 |
| LC17 | 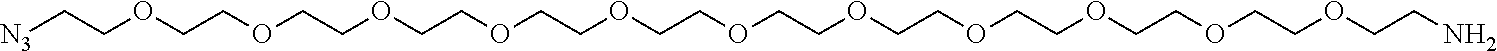 [SMILES: [N-]=[N+]=NCCOCCOCCOCCOCCOCCOCCOCCOCCOCCOCCOCCN] |
| LC18 | 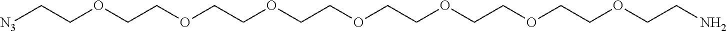 [SMILES: [N-]=[N+]=NCCOCCOCCOCCOCCOCCOCCOCCN] |
| LC19 | 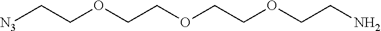 [SMILES: [N-]=[N+]=NCCOCCOCCOCCN] |
| LC20 | 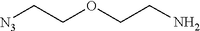 |
| LC21 | 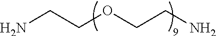 |
| LC22 | 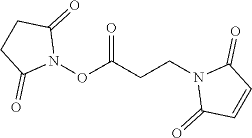 [SMILES: O=C(CCN1C(=O)C=CC1=O)ON1C(=O)CCC1=O] |
| LC23 | 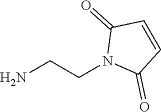 |
| LC24 | 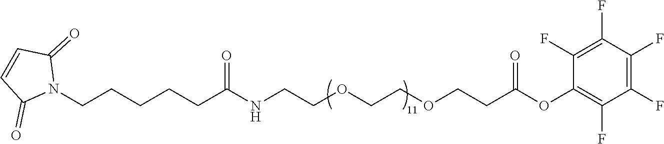 |
| LC25 | 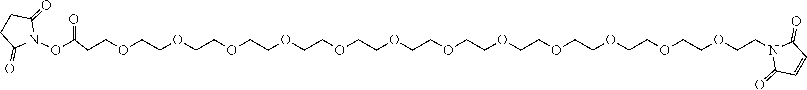 [SMILES: O=C(CCOCCOCCOCCOCCOCCOCCOCCOCCOCCOCCOCCOCCN1C(=O)C=CC1=O)ON1C(=O)CCC1=O] |
| LC26 | 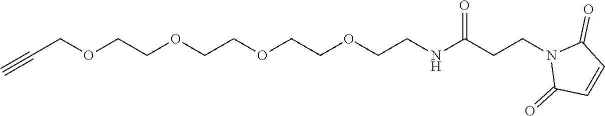 [SMILES: C#CCOCCOCCOCCOCCNC(=O)CCN1C(=O)C=CC1=O] |
| LC27 | 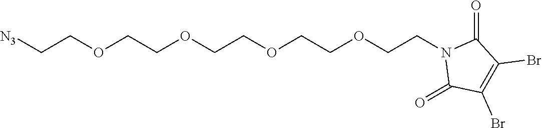 [SMILES: [N-]=[N+]=NCCOCCOCCOCCOCCN1C(=O)C(Br)=C(Br)C1=O] |
| LC28 | 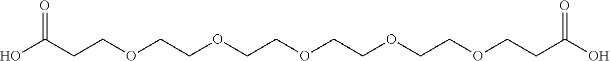 [SMILES: O=C(O)CCOCCOCCOCCOCCOCCC(=O)O] |
| LC29 | 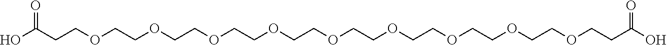 [SMILES: O=C(O)CCOCCOCCOCCOCCOCCOCCOCCOCCOCCC(=O)O] |
| LC30 | 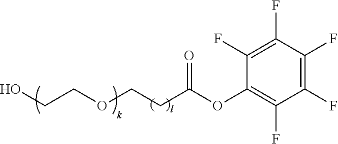 |
| | k = 4, l = 0 |
| | k = 0, l = 12 |
| | k = 2, l = 6 |
| LC31 | 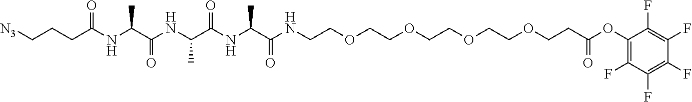 [SMILES: C[C@H](NC(=O)CCCN=[N+]=[N-])C(=O)N[C@@H](C)C(=O)N[C@@H](C)C(=O)NCCOCCOCCOCCOCCC(=O)Oc1c(F)c(F)c(F)c(F)c1F] |
| LC32 | 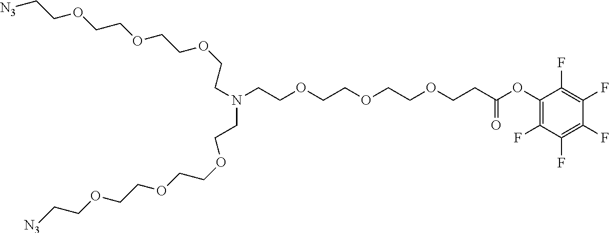 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F] |
| LC33 | 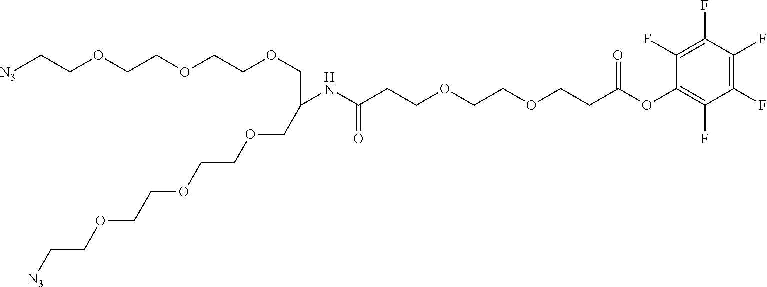 [SMILES: [N-]=[N+]=NCCOCCOCCOCC(COCCOCCOCCN=[N+]=[N-])NC(=O)CCOCCOCCC(=O)Oc1c(F)c(F)c(F)c(F)c1F] |
| LC34 | 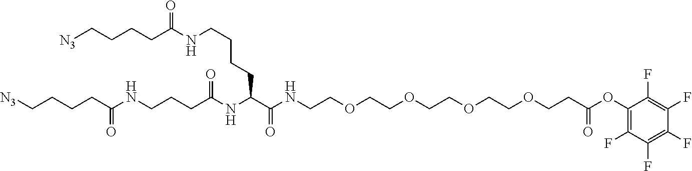 [SMILES: [N-]=[N+]=NCCCCC(=O)NCCCC[C@H](NC(=O)CCCNC(=O)CCCCN=[N+]=[N-])C(=O)NCCOCCOCCOCCOCCC(=O)Oc1c(F)c(F)c(F)c(F)c1F] |
| LC35 | 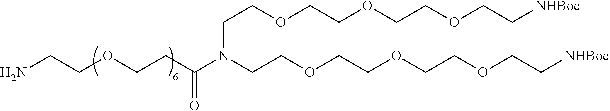 |
| LC36 | 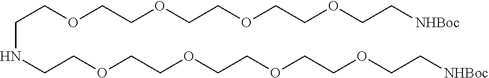 [SMILES: CC(C)(C)OC(=O)NCCOCCOCCOCCOCCNCCOCCOCCOCCOCCNC(=O)OC(C)(C)C] |
| LC37 | 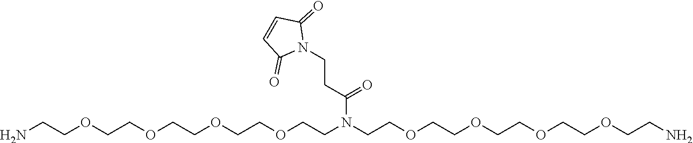 [SMILES: NCCOCCOCCOCCOCCN(CCOCCOCCOCCOCCN)C(=O)CCN1C(=O)C=CC1=O] |
| LC38 | 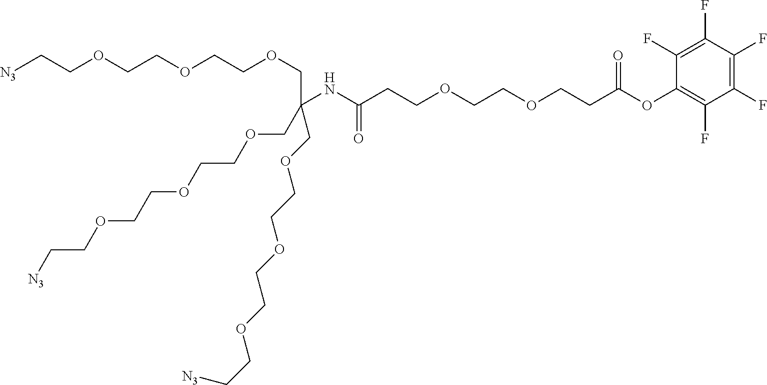 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F] |
| LC39 | 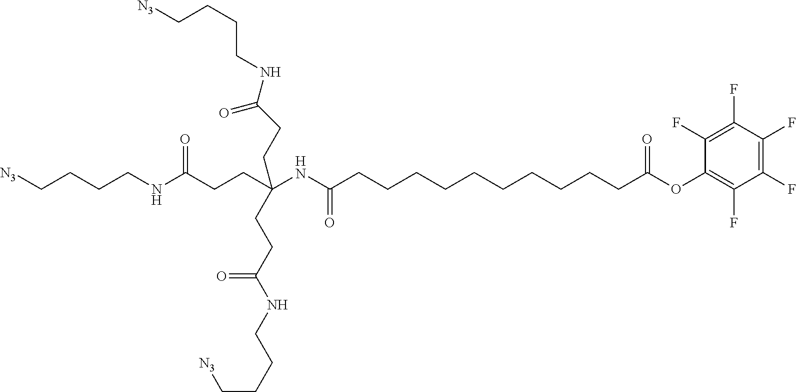 [SMILES: [N-]=[N+]=NCCCCNC(=O)CCC(CCC(=O)NCCCCN=[N+]=[N-])(CCC(=O)NCCCCN=[N+]=[N-])NC(=O)CCCCCCCCCCC(=O)Oc1c(F)c(F)c(F)c(F)c1F] |
| LC40 | 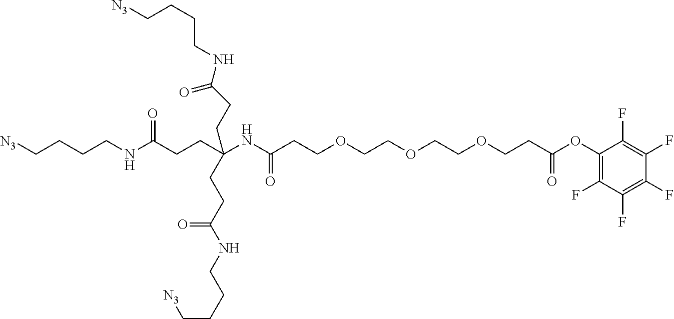 [SMILES: [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)Oc1c(F)c(F)c(F)c(F)c1F] |
| LC41 | 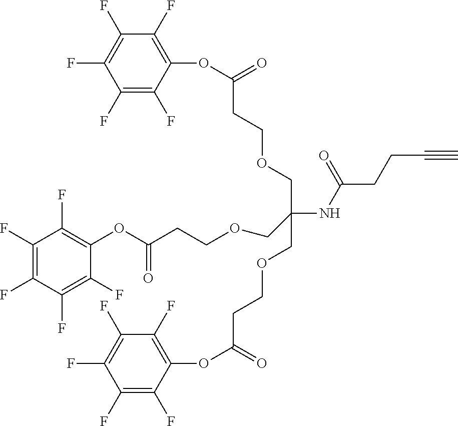 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F] |
| LC42 | 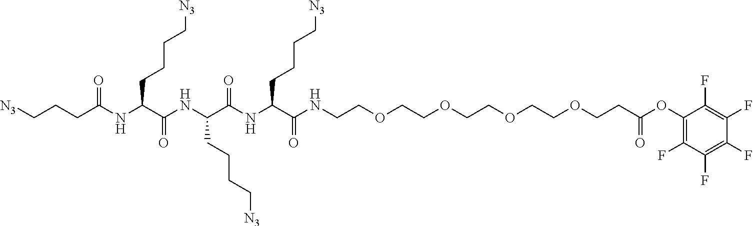 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC43 | 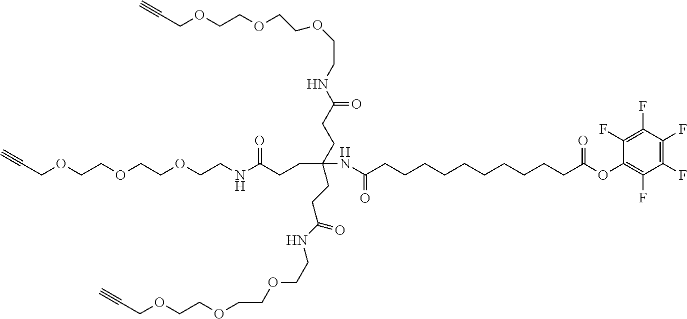 [SMILES: C#CCOCCOCCOCCNC(=O)CCC(CCC(=O)NCCOCCOCCOCC#C)(CCC(=O)NCCOCCOCCOCC#C)NC(=O)CCCCCCCCCCC(=O)Oc1c(F)c(F)c(F)c(F)c1F] |
| LC44 | 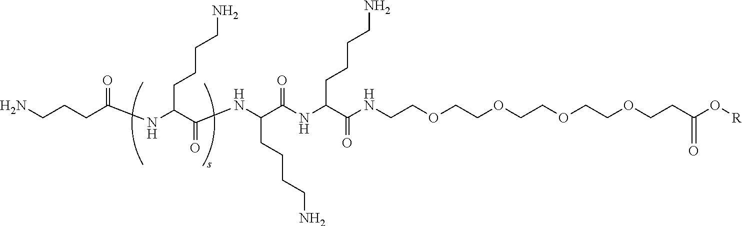 |
| | where R is H, or protecting group, and s is 1, 2, 3, 5-10, or 10-100, or 20-50. |
| LC45 | 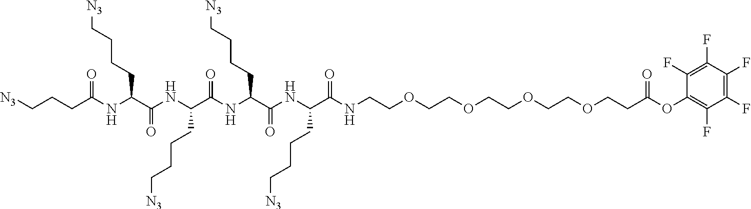 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC46 | 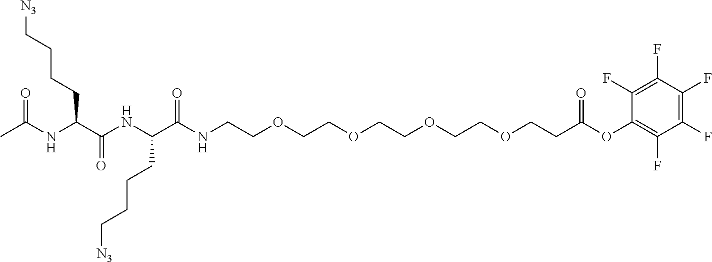 [SMILES: CC(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC47 | 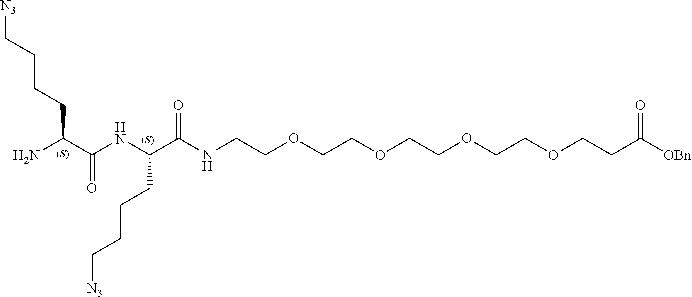 |
| LC48 | 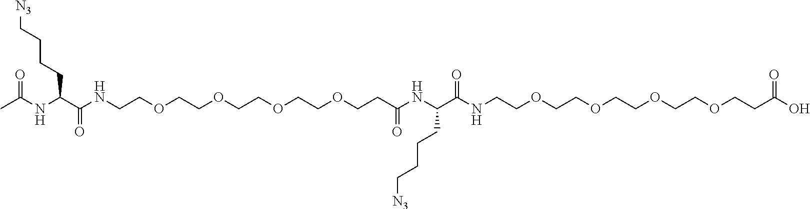 [SMILES: CC(=O)N[C@@H](CCCCN=[N+]=[N-])C(=O)NCCOCCOCCOCCOCCC(=O)N[C@@H](CCCCN=[N+]=[N-])C(=O)NCCOCCOCCOCCOCCC(=O)O] |
| LC49 LC50 LC51 LC52 LC53 | 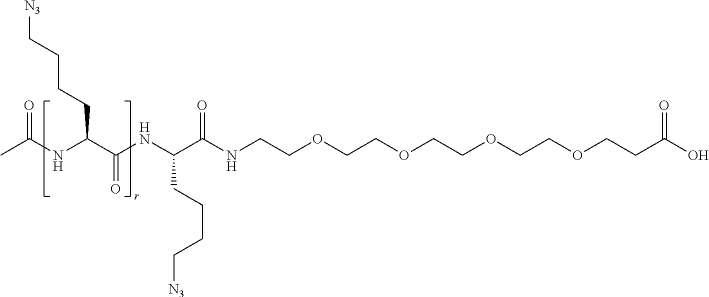 |
| | LC49 where r is 0 |
| | LC50 where r is 1 |
| | LC51 where r is 2 |
| | LC52 where r is 3 |
| | LC53 where r is 4 |
| LC54 LC55 LC56 LC57 LC58 LC59 LC60 | 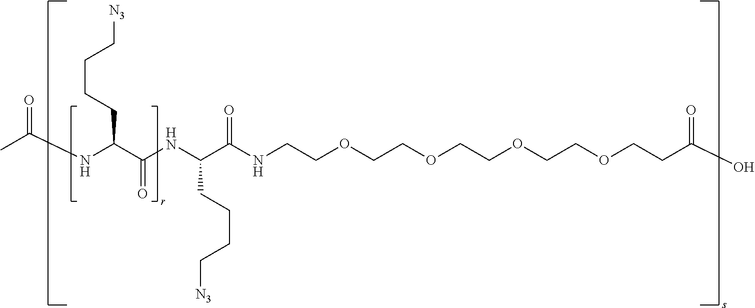 |
| | LC54 where r is 0, s is 2 |
| | LC55 where r is 1, s is 2 |
| | LC56 where r is 2, s is 2 |
| | LC57 where r is 0, s is 3 |
| | LC58 where r is 1, s is 3 |
| | LC59 where r is 2, s is 3 |
| | LC60 where r is 0-4, s is 4-20 |
| L61 L62 | 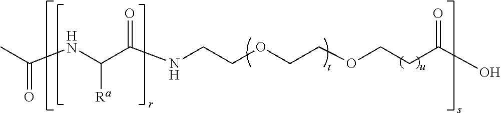 |
| | 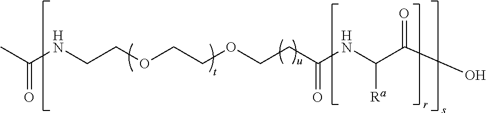 |
| | where r is 1-3, t is 3-5, u is 0 or 1, and s is 2-5 |

### Chemoselective Ligation Group

A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, tyrosine specific conjugation chemistry (e.g., e-Y-CLICK), methionine specific conjugation chemistry (e.g., oxaziridine-based or ReACT chemistry), reductive amination, dialkyl squarate chemistry, etc.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments of formula (I), Y is a reactive moiety capable forming a covalent bond to a polypeptide (e.g., with an amino acid sidechain of a polypeptide having a compatible reactive group). The reactive moiety can be referred to as a chemoselective ligation group.

In certain embodiments of formula (I), Y is a thio-reactive chemoselective ligation group (e.g., as described in Table 10). In some cases, Y can produce a residual moiety Z resulting from the covalent linkage of a thiol-reactive chemoselective ligation group to one or more cysteine residue(s) of a protein, e.g., Ab.

In certain embodiments of formula (I), Y is a Cys-reactive chemoselective ligation group (e.g., a maleimide derivative as described in table 10). In some cases, the Cys-reactive chemoselective ligation group includes a maleimide group. In some embodiments, the chemoselective ligation group includes a maleimide group of Table 22, e.g., mal-1 to mal-7.

In certain embodiments of formula (I), Y is an amino-reactive chemoselective ligation group (e.g., as described in Table 10). In some cases, Y can produce a residual moiety Z resulting from the covalent linkage of an amine-reactive chemoselective ligation group to one or more lysine residue(s) a protein, e.g., Ab.

In certain embodiments of formula (I), Y is a Lys-reactive chemoselective ligation group (e.g., an active ester as described in Table 10). In some embodiments the Lys-reactive chemoselective ligation group is a PFP ester.

Exemplary chemoselective ligation groups, and synthetic precursors thereof, which may be adapted for use in the compounds of this disclosure are shown in Table 10.

TABLE 10
Exemplary chemoselective ligation groups and precursors

| Groups | Exemplary structures |
|---|---|
| carboxylic acid or active ester | 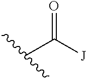 |
| | where J is selected from —OH, —Cl, —Br, —I, —F, —OH, —O—N-succinimide, |
| | —O-(4-nitrophenyl), —O-pentafluorophenyl, —O-tetrafluorophenyl, and |
| | —O—C(O)-OR^{J′}, and R^{J′} is -C₁-C₈ alkyl or -aryl, |
| | 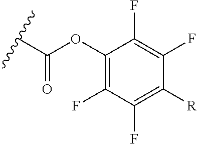 |
| | R is H or F, |
| | 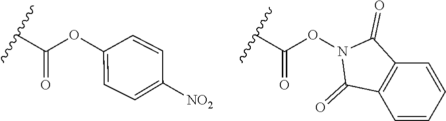 [SMILES: *C(=O)Oc1ccc([N+](=O)[O-])cc1 ; *C(=O)ON1C(=O)c2ccccc2C1=O] |
| | 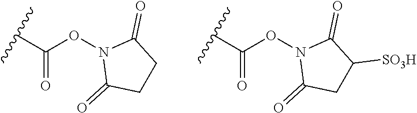 [SMILES: *C(=O)ON1C(=O)CCC1=O ; *C(=O)ON1C(=O)CC(S(=O)(=O)O)C1=O] |
| | 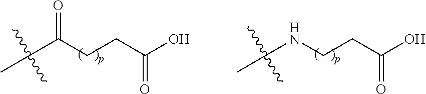 |
| | where p is 0 to 6 |
| maleimide | 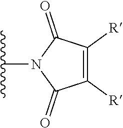 |
| | where each R′ is independently hydrogen or halogen (e.g., bromo) |
| | 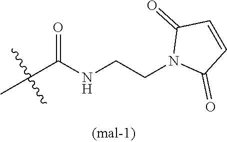 [SMILES: *C(=O)NCCN1C(=O)C=CC1=O] |
| | 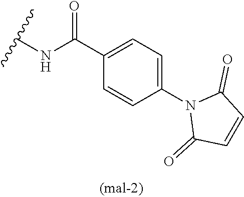 [SMILES: *NC(=O)c1ccc(N2C(=O)C=CC2=O)cc1] |
| | 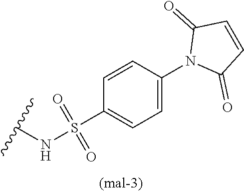 [SMILES: *NS(=O)(=O)c1ccc(N2C(=O)C=CC2=O)cc1] |
| | 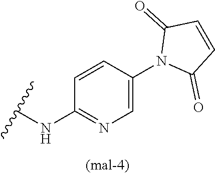 [SMILES: *Nc1ccc(N2C(=O)C=CC2=O)cn1] |
| | 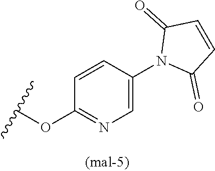 [SMILES: *Oc1ccc(N2C(=O)C=CC2=O)cn1] |
| | 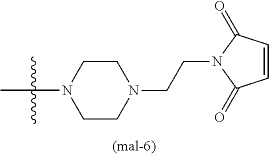 [SMILES: *N1CCN(CCN2C(=O)C=CC2=O)CC1] |
| | 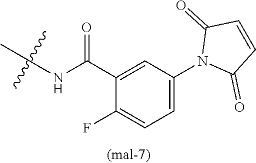 [SMILES: *NC(=O)c1cc(N2C(=O)C=CC2=O)ccc1F] |
| | 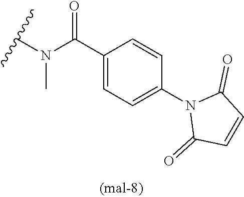 [SMILES: *N(C)C(=O)c1ccc(N2C(=O)C=CC2=O)cc1] |
| | 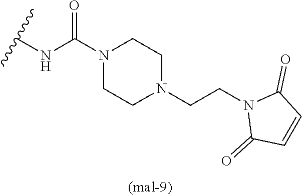 [SMILES: *NC(=O)N1CCN(CCN2C(=O)C=CC2=O)CC1] |
| | 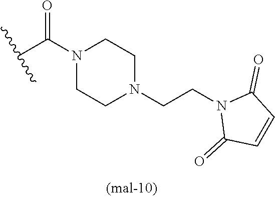 [SMILES: *C(=O)N1CCN(CCN2C(=O)C=CC2=O)CC1] |
| | 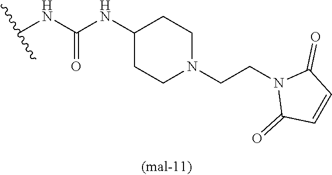 [SMILES: *NC(=O)NC1CCN(CCN2C(=O)C=CC2=O)CC1] |
| | 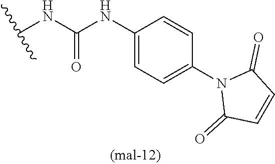 [SMILES: *NC(=O)Nc1ccc(N2C(=O)C=CC2=O)cc1] |
| | 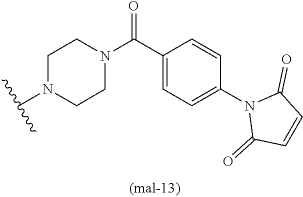 [SMILES: *N1CCN(C(=O)c2ccc(N3C(=O)C=CC3=O)cc2)CC1] |
| | 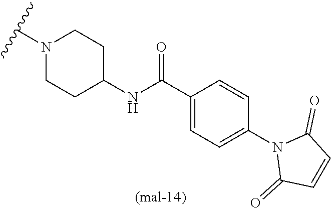 [SMILES: *N1CCC(NC(=O)c2ccc(N3C(=O)C=CC3=O)cc2)CC1] |
| isocyanate or | —NCS |
| isothiocyanate | —NCO |
| | 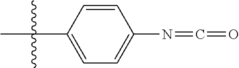 [SMILES: *c1ccc(N=C=O)cc1] |
| | 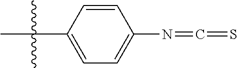 [SMILES: *c1ccc(N=C=S)cc1] |
| alkyl halide alkyl tosylate | 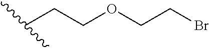 [SMILES: *CCOCCBr] |
| | 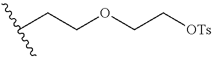 [SMILES: *CCOCCOS(=O)(=O)c1ccc(C)cc1] |
| aldehyde | 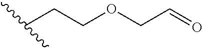 [SMILES: *CCOCC=O] |
| haloacetamide or alpha-leaving group acetamide | 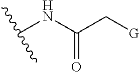 |
| | where G is selected from —Cl, —Br, —I, —O-mesyl, and —O-tosyl |
| | 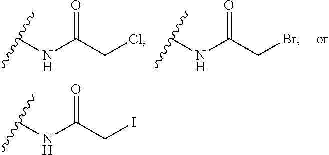 |
| haloacetamide or alpha-leaving group acetamide | 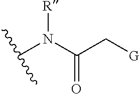 |
| | where G is selected from —Cl, —Br, —I, —O-mesyl, and —O-tosyl; R″ is |
| | (C₁-C₆)alkyl, heterocyclyl (e.g., 4-tetrahydro2H-pyran), or aryl; e.g.: |
| | 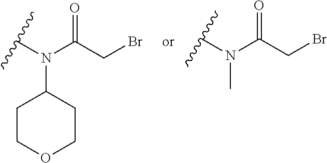 |
| 2-sulfonylpyridine | 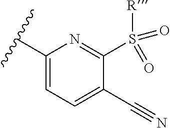 |
| | where R″′ is alkyl |
| diazirine | 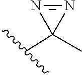 [SMILES: *C1(C)N=N1] |
| sulfonyl halide or vinyl sulfone | 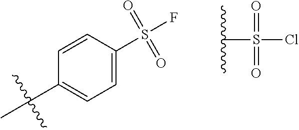 [SMILES: *c1ccc(S(=O)(=O)F)cc1 ; *S(=O)(=O)Cl] |
| | 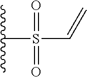 [SMILES: *S(=O)(=O)C=C] |
| hydrazide hydrazino hydroxylamino | 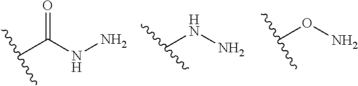 |
| pyridyl disulfide | 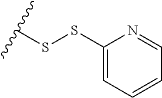 [SMILES: *SSc1ccccn1] |
| (HIPS) hydrazinyl- indolyl group, or (aza-HIPS) hydrazinyl- pyrrolo-pyridinyl group | 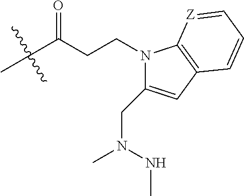 |
| | where Z is CH or N |
| alkyne or cyclooctyne | 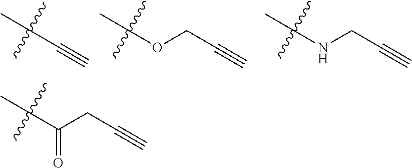 |
| | 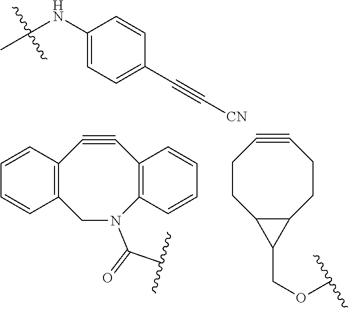 |
| azide | 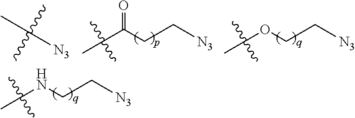 |
| | where p is 0 to 6 and where q is 1 to 6 |
| amine | 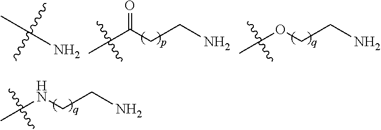 |
| | where p is 0 to 6 and where q is 1 to 6 |

In Table 10, the [symbol] can represent a point of attachment of Y to a linking moiety or a linked X moiety.

Table 11 shows exemplary residual moieties, wherein the “***” indicates the point of attachment of Y.

TABLE 11
Exemplary residual moieties from chemoselective ligation groups and precursors

| Groups | Exemplary residual moieties |
|---|---|
| carboxylic acid or active ester | 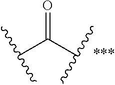 [SMILES: *C(*)=O] |
| maleimide | 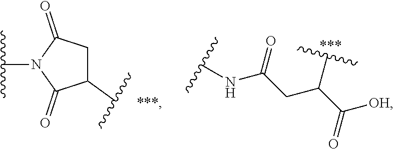 [SMILES: *C1CC(=O)N(*)C1=O ; *NC(=O)CC(*)C(=O)O] |
| | and/or |
| | 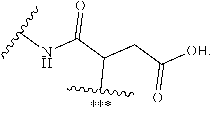 [SMILES: *NC(=O)C(*)CC(=O)O] |

TABLE 11
Exemplary residual moieties from
chemoselective ligation groups and precursors

| | |
|---|---|
| isocyanate or | Urea or thiourea |
| isothiocyanate | |
| alkyl halide | Direct bond |
| alkyl tosylate | |
| aldehyde | imine |
| haloacetamide or | Direct bond |
| alpha-leaving group | |
| acetamide | |

Exemplary ASGPR-Binding Compounds with Chemoselective Ligation Group for Preparing Conjugates

This disclosure includes compounds of formula (I) which compounds can be prepared from a precursor ligand-linker compound including:
(1) one or more particular ASGPR ligand (X) (e.g., as described herein, such as ligands X1-X20 of Tables 1-5) or a particular ASGPR ligand (X) (e.g., as described herein),
(2) a linker including one or more linking moieties (e.g., as described herein, such as any one or more of the linking moieties of Tables 6 to 8); and
(3) a chemoselective ligation group (Y) e.g., as described herein, such as any one of the groups of Table 10).

Table 12 illustrates various monovalent ASGPR ligand-linker compounds for use in preparing conjugates of the disclosure.

TABLE 12
Exemplary ASGPR ligand-linker compounds for use in conjugates
Xₙ—L—Y

| | | | | effective length | Chemoselective |
|---|---|---|---|---|---|
| Cmpd # | X | L | n | L to Y conjugate | ligation |
| 1101 (1-117) | X1 | | 1 | 10 | PFP ester |
| 1102 (1-115) | X1 | | 1 | 19 | PFP ester |
| 1103 | X1 | | 1 | 25 | PFP ester |
| 1104 (1-133) | X1 | | 1 | 29 | maleimide |
| 1105 | X1 | | 1 | 28 | PFP ester |
| 1106 (1-112) | X1 | | 1 | 26 | PFP ester |
| 1107 (1-146) | X1 | | 1 | 35 | PFP ester |
| 1108 (I-118) | X6 | | 1 | 10 | PFP ester |
| 1109 (1-116) | X6 | | 1 | 19 | PFP ester |
| 1110 (1-113) | X6 | | 1 | 26 | NHS ester |
| 1111 (1-147) | X5 | L10 | 1 | 27 | PFP ester |
| 1112 (I-148) | X5 | L1 | 1 | 26 | PFP ester |
| 1113 (1-149) | X1 | L11 | 1 | 23 | PFP ester |
| 1114 (1-150) | X1 | L11 | 1 | 26 | PFP ester |
| 1115 (1-151) | X1 | L11 | 1 | 29 | PFP ester |

PFP is pentafluorophenyl
TFP is tetrafluorophenyl
NHS is N-hydroxysuccinimde ester

Tables 13 illustrates various multivalent ASGPR ligand-linker compounds for use in conjugates of the disclosure.

TABLE 13
Exemplary Multimeric ASGPR Ligand-linker compounds
Xₙ—L—Y

| Cmpd # | X | n | X¹ to branch length | branch to Y length | Y |
|---|---|---|---|---|---|
| 1218 (1-143) | X4 | 3 | 14 to C | 12 to C═O | PFP ester |
| 1303 (I-136) | X8 | 3 | 16 to C | 12 to C═O | PFP ester |
| 1219 (I-157) | X4 | 3 | 6 to N | 12 to C═0 | PFP ester |
| 1213 (1-137) | X1 | 3 | 15 to C | 81 to C═O | PFP ester |
| 1211 (I-129) | X1 | 3 | 15 to C | 33 to C═O | PFP ester |
| 1203 (1-144) | X1 | 2 | 15 to CH | 12 to C═O | PFP ester |
| 1215 (1-141) | X2 | 3 | 15 to C | 12 to C═O | PFP ester |
| 1208 (I-145) | X1 | 3 | 15 to C | 16 to C═O | PFP ester |
| 1216 (I-140) | X3 | 3 | 18 to C | 12 to C═O | PFP ester |
| 1401 (I-153) | X10 | 3 | 14 to C | 12 to C═O | PFP ester |
| 1204 (I-111) | X1 | 2 | 16 to N | 11 to C═O | PFP ester |
| 1402 (1-154) | X11 | 3 | 19 to C | 12 to C═O | PFP ester |
| 1403 (I-155) | X12 | 3 | 19 to C | 12 to C═O | PFP ester |
| 1246 | X2 | 3 | 17 to C | 12 to phenyl group | mal-2 |
| | (α) | | | | |
| 1248 | X3 | 3 | 15 to C | 17 to piperazine | mal-6 |
| | (α) | | | | |
| 1250 | X3 | 3 | 17 to C | 19 to C═O | mal-1 |
| | (α) | | | | |
| 1225A | X2 | 3 | 16 to C | 12 to C═O | mal-1 |
| | (α) | | | | |
| 1251 | X3 | 3 | 17 to C | 12 to C═O | mal-1 |
| | (α) | | | | |
| 1252 | X1 | 3 | 18 to C | 18 to phenyl | mal-2 |
| 1249 | X3 | 3 | 17 to C | 17 to piperazine | mal-6 |
| | (α) | | | | |
| 1258 | X3 | 3 | 21 to C | 18 to phenyl | mal-2 |
| | (α) | | | | |
| 1921 | X22 | 3 | 18 to C | 18 to phenyl | mal-2 |
| 1253 | X2 | 3 | 15 to C | 18 to phenyl | mal-2 |
| | (α) | | | | |
| 1255 | X3 | 3 | 16 to C | 18 to phenyl | mal-2 |
| | (β) | | | | |
| 1257 | X2 | 3 | 17 to C | 19 to phenyl | mal-7 |
| | (α) | | | | |
| 1247 | X3 | 3 | 18 to C | 18 to phenyl | mal-2 |
| | (α) | | | | |
| 1254 | X2 | 3 | 17 to C | 18 to phenyl | mal-2 |
| | (α) | | | | |
| 1259 | X3 | 3 | 16 to C | 18 to phenyl | mal-2 |
| | (α) | | | | |
| 1915A | X25 | 3 | 19 to C | 18 to phenyl | mal-2 |

Tables 14-17 illustrate several exemplary ASGPR binding compounds of this disclosure that include a chemoselective ligation group, or a precursor thereof. It is understood that this disclosure includes Y (e.g., as described herein) conjugates of each of the exemplary compounds of Tables 14-17. For example, conjugates where the chemoselective ligation group has been conjugated to a different Y, such as an antibody or antibody fragment for a target protein.

The chemoselective ligation group of such compounds can be utilized to connect to another Y moiety of interest (e.g., as described below). It is understood that any of these compounds can also be prepared de novo to include an alternative Y moiety of interest (e.g., as described below) rather than the chemoselective ligation group. In some embodiments, such compounds are referred to as a conjugate, e.g., a biomolecule conjugate that specifically binds a target protein.

TABLE 14
Example ASGPR binding compounds having chemoselective ligation group

| # | Structure |
|---|---|
| 1101 (I- 117) | 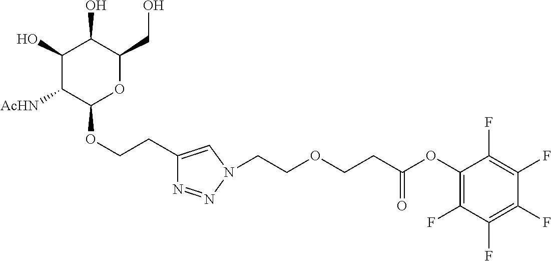 [SMILES: CC(=O)N[C@H]1[C@H](OCCc2cn(CCOCCC(=O)Oc3c(F)c(F)c(F)c(F)c3F)nn2)O[C@H](CO)[C@H](O)[C@@H]1O] |
| 1102 (I- 115) | 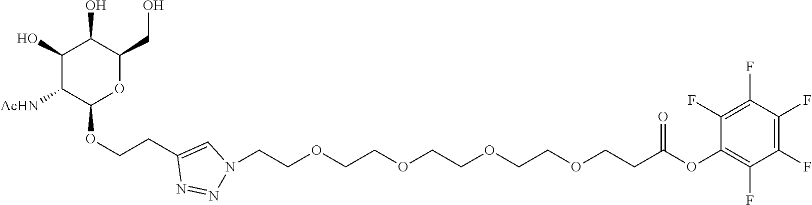 [SMILES: CC(=O)N[C@H]1[C@H](OCCc2cn(CCOCCOCCOCCOCCC(=O)Oc3c(F)c(F)c(F)c(F)c3F)nn2)O[C@H](CO)[C@H](O)[C@@H]1O] |
| 1103 | 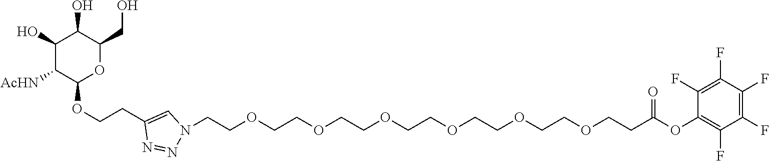 [SMILES: CC(=O)N[C@H]1[C@H](OCCc2cn(CCOCCOCCOCCOCCOCCOCCC(=O)Oc3c(F)c(F)c(F)c(F)c3F)nn2)O[C@H](CO)[C@H](O)[C@@H]1O] |
| 1104 (I- 133) | 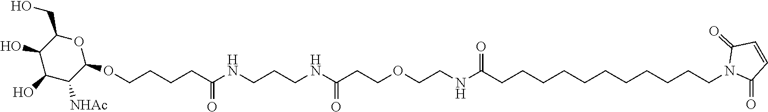 [SMILES: CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O] |
| 1105 | 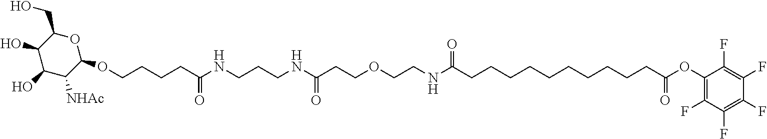 [SMILES: CC(=O)N[C@H]1[C@H](OCCCCC(=O)NCCCNC(=O)CCOCCNC(=O)CCCCCCCCCCC(=O)Oc2c(F)c(F)c(F)c(F)c2F)O[C@H](CO)[C@H](O)[C@@H]1O] |
| 1106 (I- 112) | 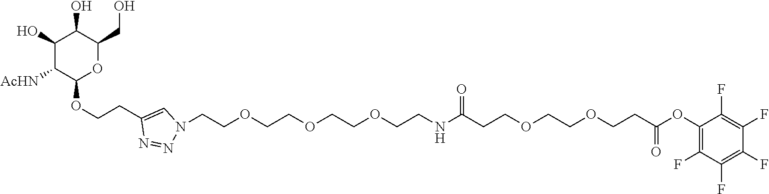 [SMILES: CC(=O)N[C@H]1[C@H](OCCc2cn(CCOCCOCCOCCNC(=O)CCOCCOCCC(=O)Oc3c(F)c(F)c(F)c(F)c3F)nn2)O[C@H](CO)[C@H](O)[C@@H]1O] |
| 1107 (I- 146) | 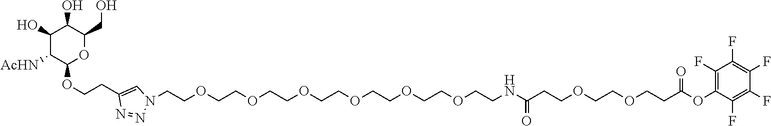 [SMILES: CC(=O)N[C@H]1[C@H](OCCc2cn(CCOCCOCCOCCOCCOCCOCCNC(=O)CCOCCOCCC(=O)Oc3c(F)c(F)c(F)c(F)c3F)nn2)O[C@H](CO)[C@H](O)[C@@H]1O] |
| 1108 (I- 118) | 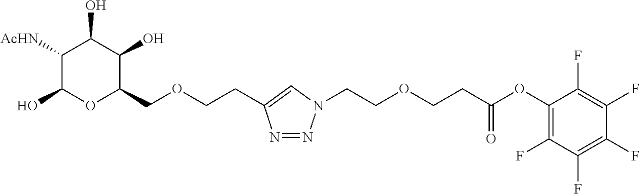 [SMILES: CC(=O)N[C@@H]1[C@@H](O)[C@@H](O)[C@@H](COCCc2cn(CCOCCC(=O)Oc3c(F)c(F)c(F)c(F)c3F)nn2)O[C@H]1O] |
| 1109 (I- 116) | 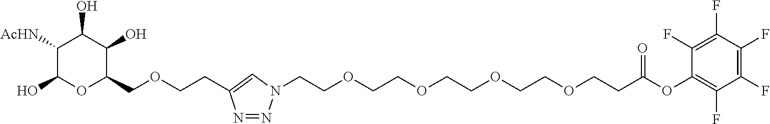 [SMILES: CC(=O)N[C@@H]1[C@@H](O)[C@@H](O)[C@@H](COCCc2cn(CCOCCOCCOCCOCCC(=O)Oc3c(F)c(F)c(F)c(F)c3F)nn2)O[C@H]1O] |
| 1110 (I- 113) | 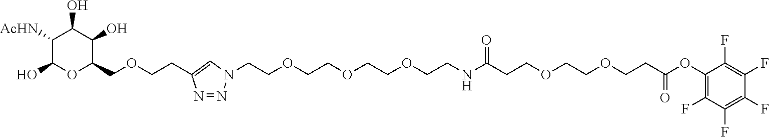 [SMILES: CC(=O)N[C@@H]1[C@@H](O)[C@@H](O)[C@@H](COCCc2cn(CCOCCOCCOCCNC(=O)CCOCCOCCC(=O)Oc3c(F)c(F)c(F)c(F)c3F)nn2)O[C@H]1O] |
| 1111 (I- 147) | 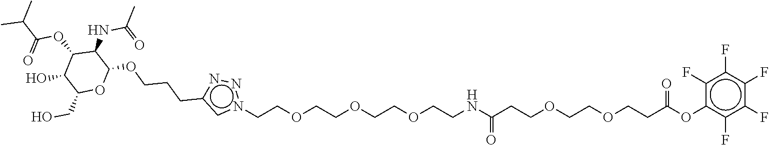 |
| 1112 (I- 148) | 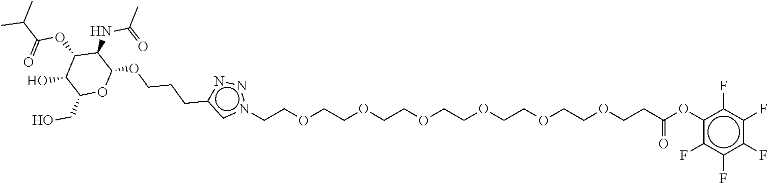 |
| 1113 (I- 149) | 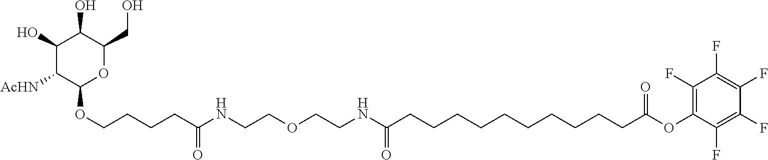 [SMILES: CC(=O)N[C@H]1[C@H](OCCCCC(=O)NCCOCCNC(=O)CCCCCCCCCCC(=O)Oc2c(F)c(F)c(F)c(F)c2F)O[C@H](CO)[C@H](O)[C@@H]1O] |
| 1114 (I- 150) | 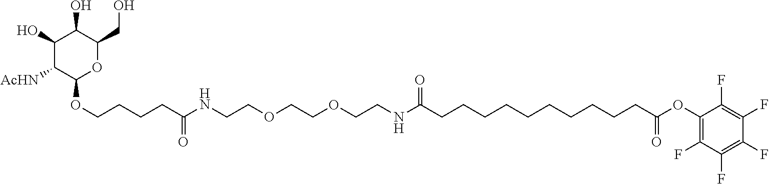 [SMILES: CC(=O)N[C@H]1[C@H](OCCCCC(=O)NCCOCCOCCNC(=O)CCCCCCCCCCC(=O)Oc2c(F)c(F)c(F)c(F)c2F)O[C@H](CO)[C@H](O)[C@@H]1O] |
| 1115 (I- 151) | 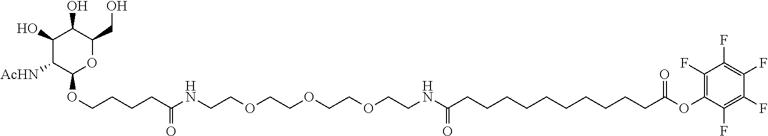 [SMILES: CC(=O)N[C@H]1[C@H](OCCCCC(=O)NCCOCCOCCOCCNC(=O)CCCCCCCCCCC(=O)Oc2c(F)c(F)c(F)c(F)c2F)O[C@H](CO)[C@H](O)[C@@H]1O] |
| 1116 (I- 164) | 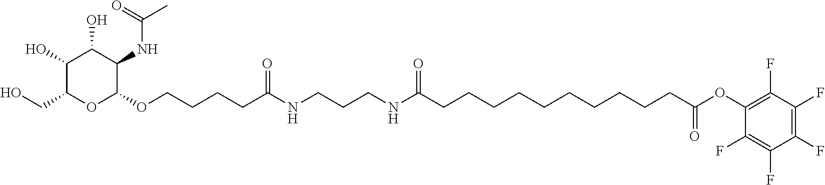 [SMILES: CC(=O)N[C@H]1[C@H](OCCCCC(=O)NCCCNC(=O)CCCCCCCCCCC(=O)Oc2c(F)c(F)c(F)c(F)c2F)O[C@H](CO)[C@H](O)[C@@H]1O] |
| 1117 (I- 168) | 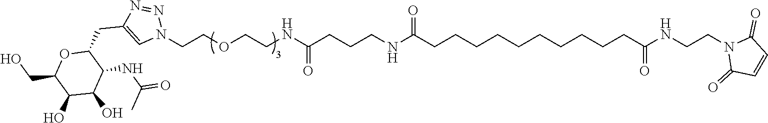 |
| 1118 (I- 169) | 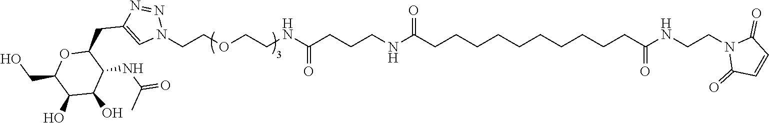 |
| 1119 | 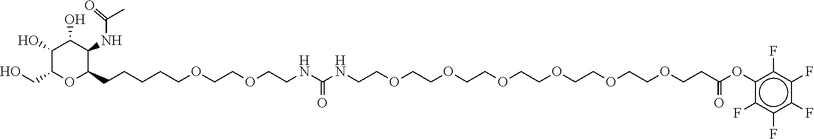 |
| 1120 | 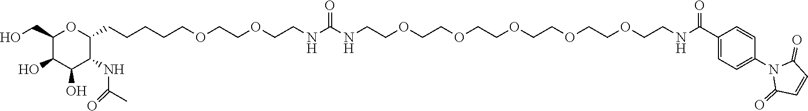 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)c1ccc(N2C(=O)C=CC2=O)cc1] |
| 1121 | 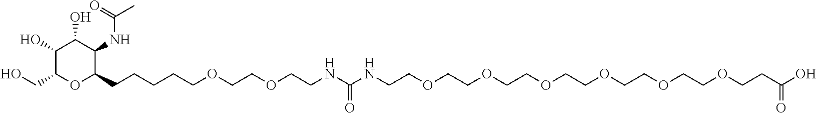 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)O] |
| 1122 | 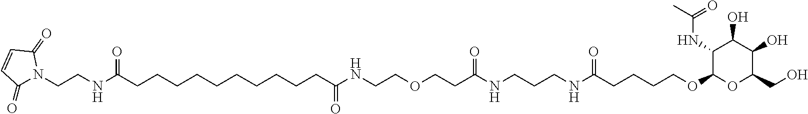 [SMILES: CC(=O)N[C@H]1[C@H](OCCCCC(=O)NCCCNC(=O)CCOCCNC(=O)CCCCCCCCCCC(=O)NCCN2C(=O)C=CC2=O)O[C@H](CO)[C@H](O)[C@@H]1O] |
| 1123 | 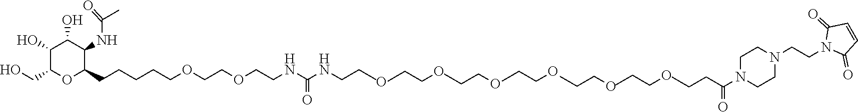 |
| 1124 | 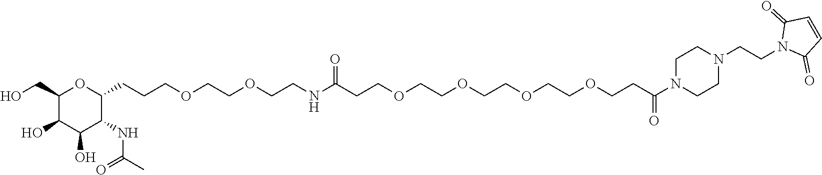 [SMILES: CC(=O)N[C@@H]1[C@@H](O)[C@@H](O)[C@@H](CO)O[C@@H]1CCCOCCOCCNC(=O)CCOCCOCCOCCOCCC(=O)N1CCN(CCN2C(=O)C=CC2=O)CC1] |

TABLE 15
Multivalent ASGPR binding compounds having chemoselective ligation group and X
group of formula (Ib)

| # | Structure |
|---|---|
| 1201 | 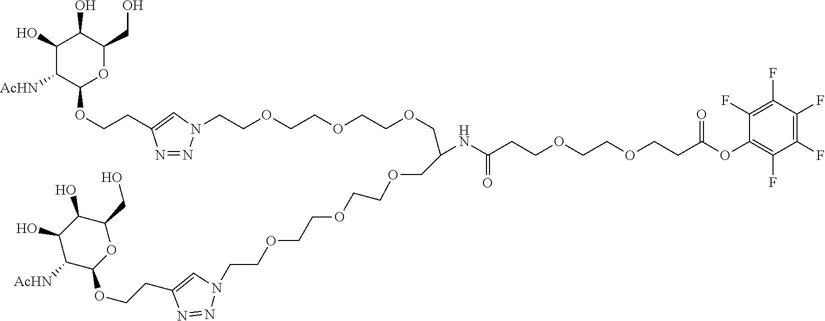 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 1202 (I-131) | 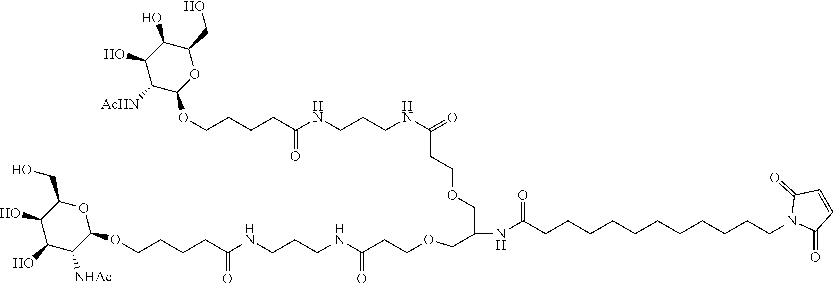 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 1203 (I-144) | 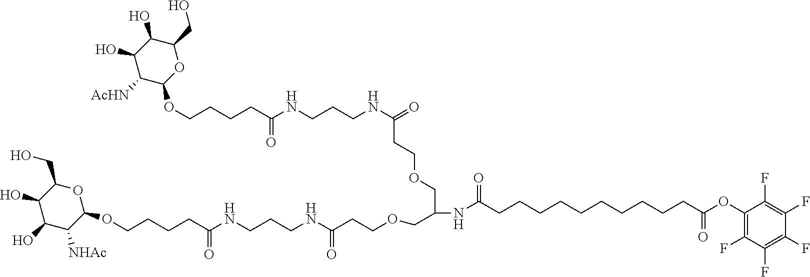 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 1204 (I-111) | 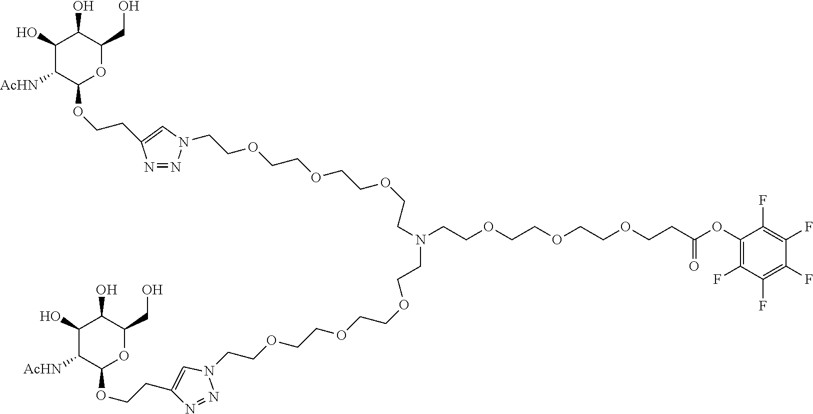 [SMILES: CC(=O)N[C@H]1[C@H](OCCc2cn(CCOCCOCCOCCN(CCOCCOCCOCCC(=O)Oc3c(F)c(F)c(F)c(F)c3F)CCOCCOCCOCCn3cc(CCO[C@@H]4O[C@H](CO)[C@H](O)[C@H](O)[C@H]4NC(C)=O)nn3)nn2)O[C@H](CO)[C@H](O)[C@@H]1O] |
| 1217 | 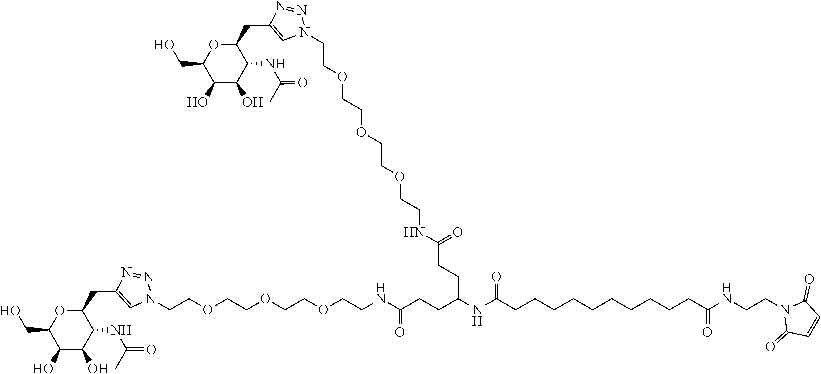 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1217 A | 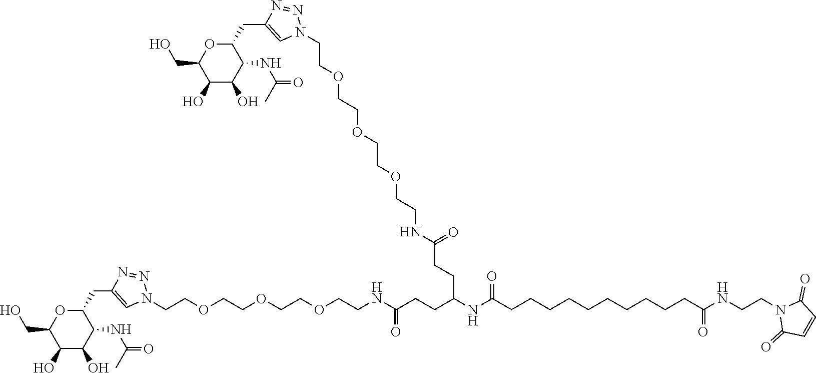 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1235 | 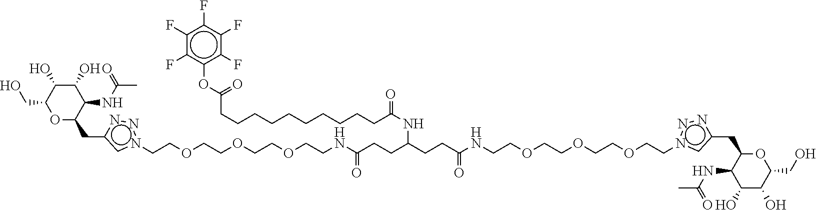 |
| 1236 | 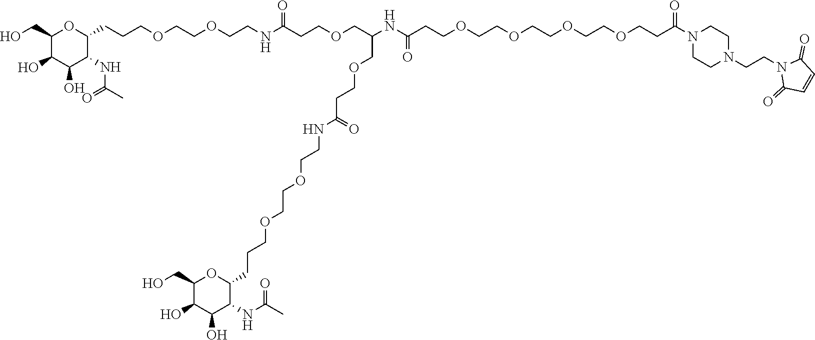 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1237 | 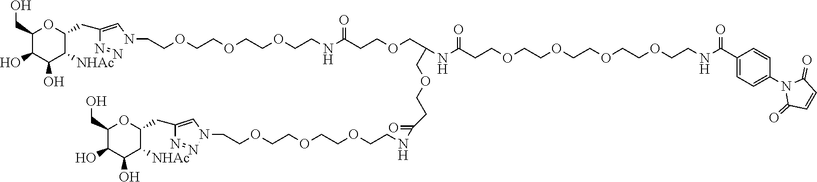 [SMILES: CC(=O)N[C@@H]1[C@@H](O)[C@@H](O)[C@@H](CO)O[C@@H]1Cc1cn(CCOCCOCCOCCNC(=O)CCOCC(COCCC(=O)NCCOCCOCCOCCn2cc(C[C@H]3O[C@H](CO)[C@H](O)[C@H](O)[C@H]3NC(C)=O)nn2)NC(=O)CCOCCOCCOCCOCCNC(=O)c2ccc(N3C(=O)C=CC3=O)cc2)nn1] |
| 1238 | 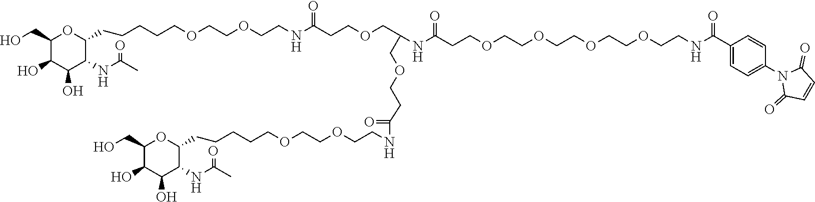 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1239 | 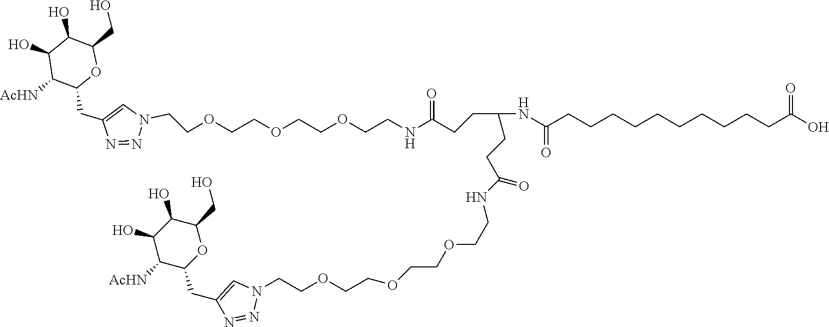 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)O)nn1] |
| 1240 | 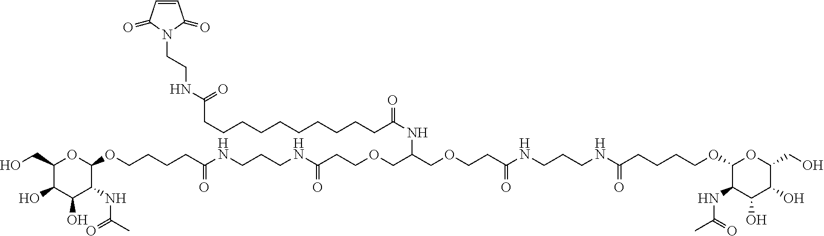 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| 1241 | 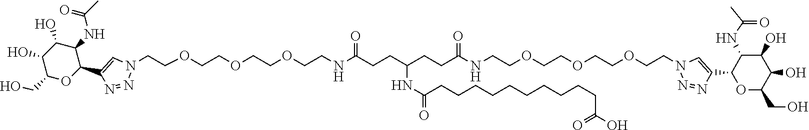 [SMILES: CC(=O)N[C@@H]1[C@@H](O)[C@@H](O)[C@@H](CO)O[C@@H]1c1cn(CCOCCOCCOCCNC(=O)CCC(CCC(=O)NCCOCCOCCOCCn2cc([C@H]3O[C@H](CO)[C@H](O)[C@H](O)[C@H]3NC(C)=O)nn2)NC(=O)CCCCCCCCCCC(=O)O)nn1] |
| 1242 | 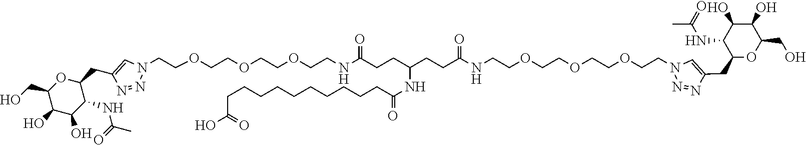 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)O)nn1] |
| 1205 (I-127) | 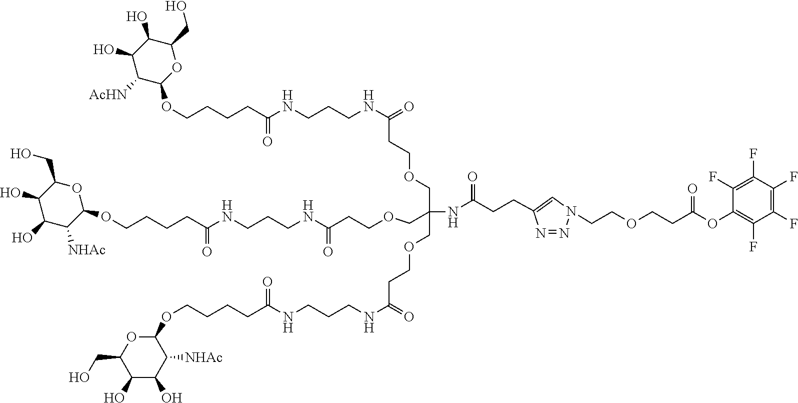 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCC(=O)Oc3c(F)c(F)c(F)c(F)c3F)nn2)O[C@H](CO)[C@H](O)[C@@H]1O] |
| 1206 | 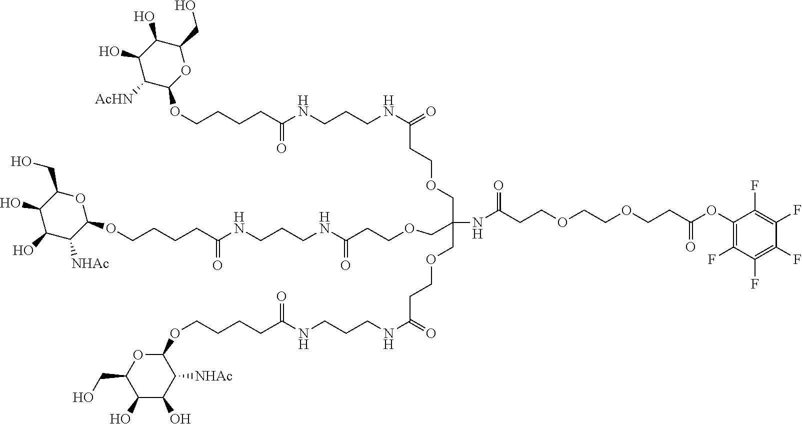 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCC(=O)Oc2c(F)c(F)c(F)c(F)c2F)O[C@H](CO)[C@H](O)[C@@H]1O] |
| 1207 (I-107) | 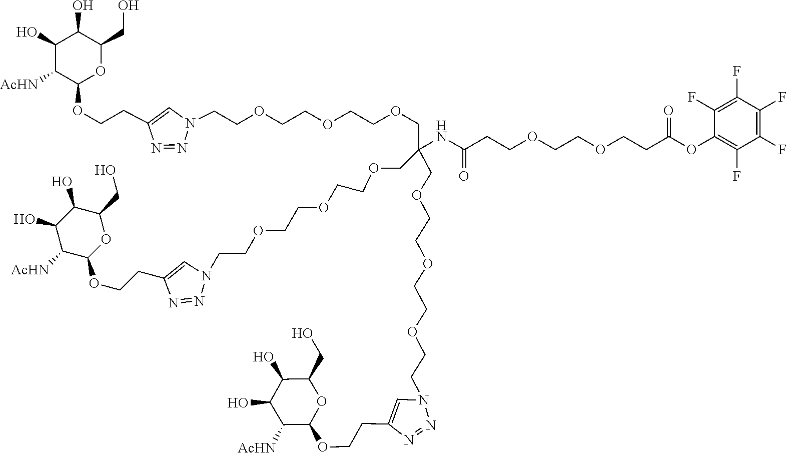 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 1208 (I-145) | 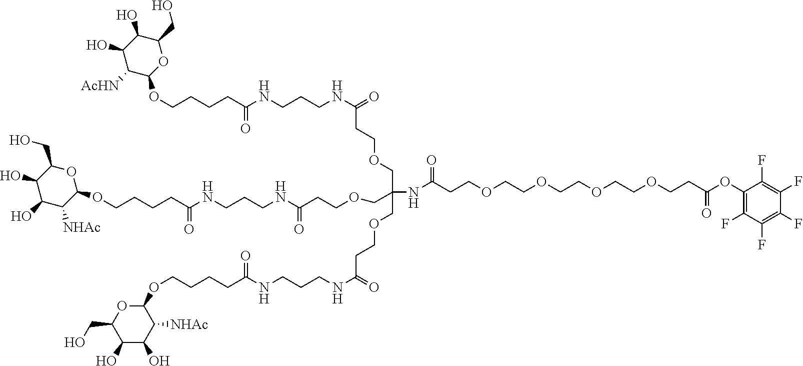 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCC(=O)Oc2c(F)c(F)c(F)c(F)c2F)O[C@H](CO)[C@H](O)[C@@H]1O] |
| 1209 (I-124) | 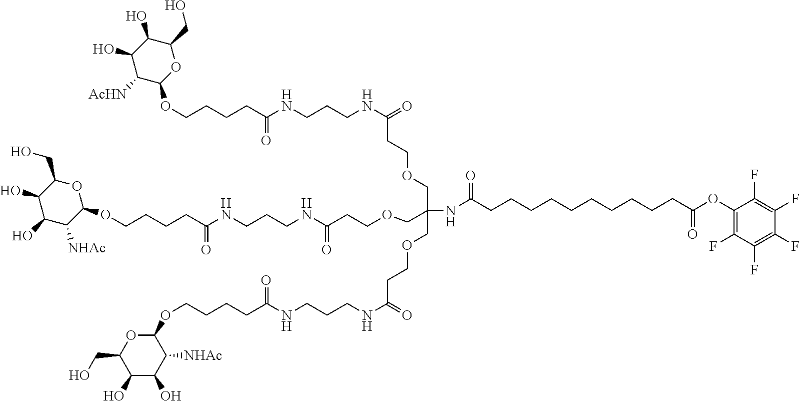 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 1210 (I-123) | 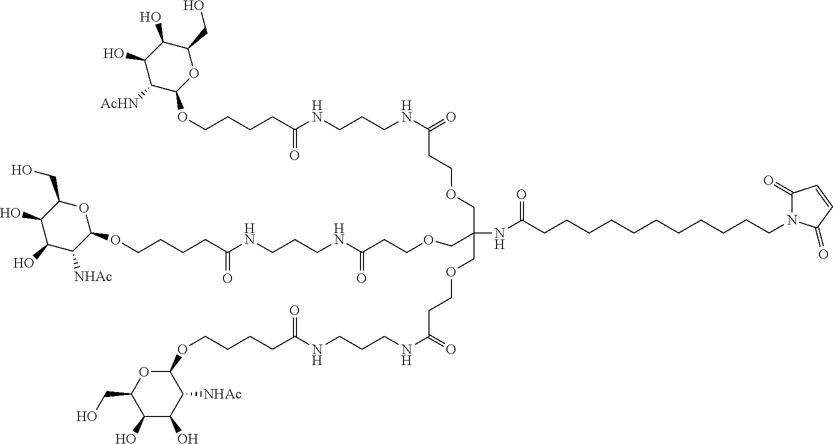 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 1210 A | 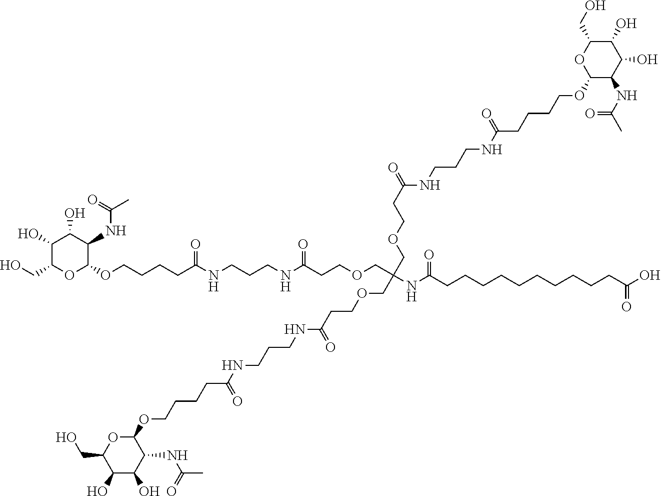 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)O)O[C@H](CO)[C@H](O)[C@@H]1O] |
| 1211 (I-129) | 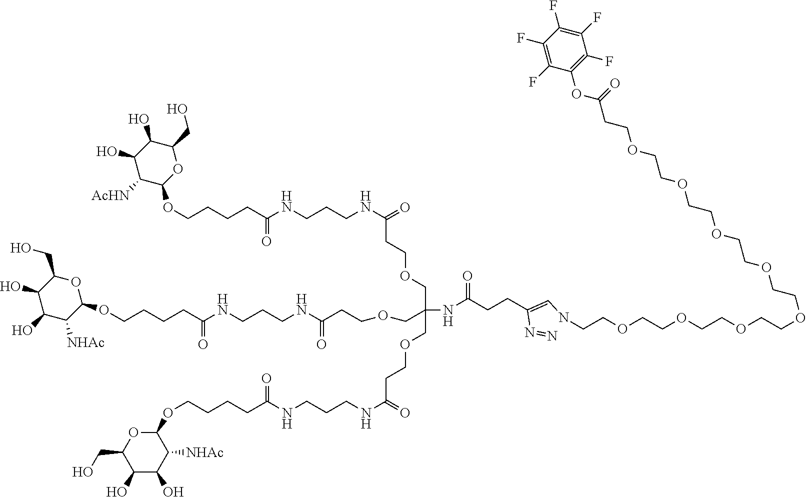 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CC/C(=C/NCCOCCOCCOCCOCCOCCOCCOCCOCCC(=O)Oc2c(F)c(F)c(F)c(F)c2F)N=N)O[C@H](CO)[C@H](O)[C@@H]1O] |
| 1212 I-125 | 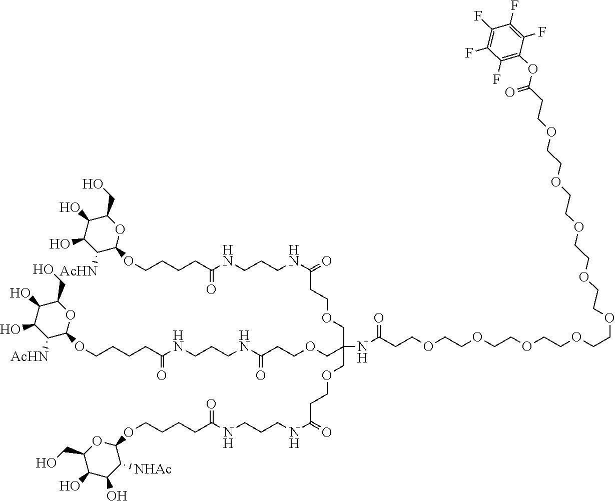 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCOCCOCCOCCOCCOCCC(=O)Oc2c(F)c(F)c(F)c(F)c2F)O[C@H](CO)[C@H](O)[C@@H]1O] |
| 1213 (I-137) | 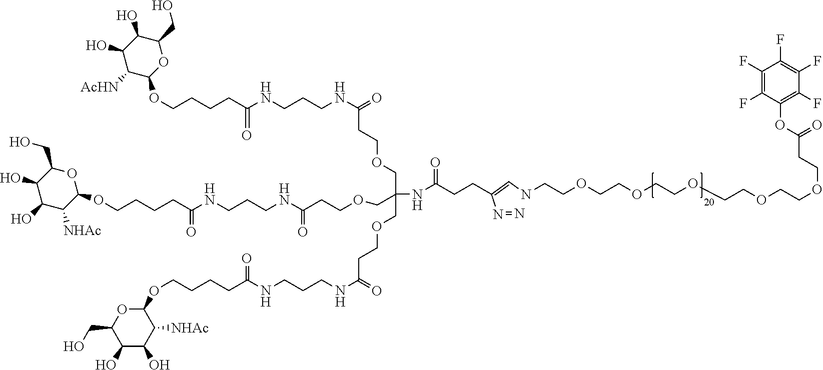 |
| 1214 (I-135) | 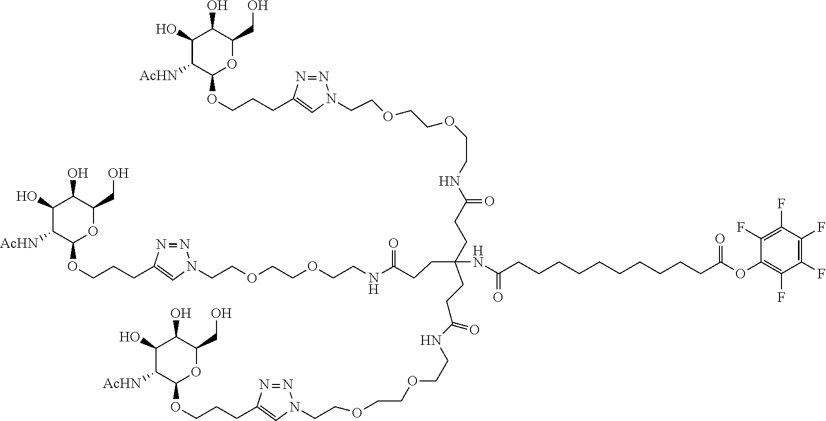 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](O)[C@H]4NC(C)=O)nn3)(CCC(=O)NCCOCCOCCn3cc(CCCO[C@@H]4O[C@H](CO)[C@H](O)[C@H](O)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1O] |
| 1215 (I-141) | 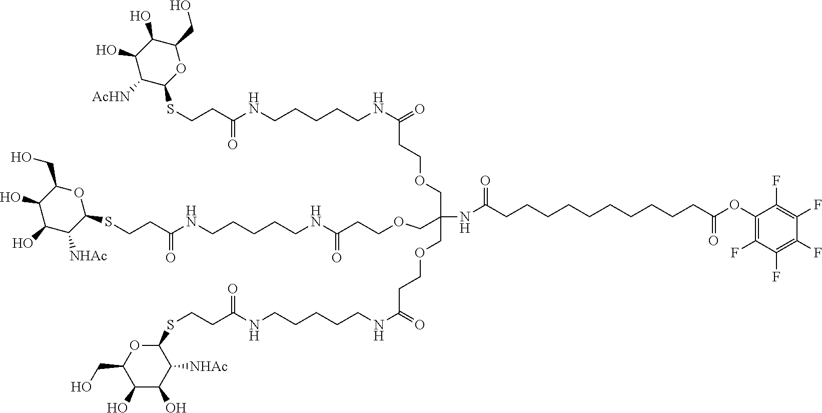 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1216 (I-140) | 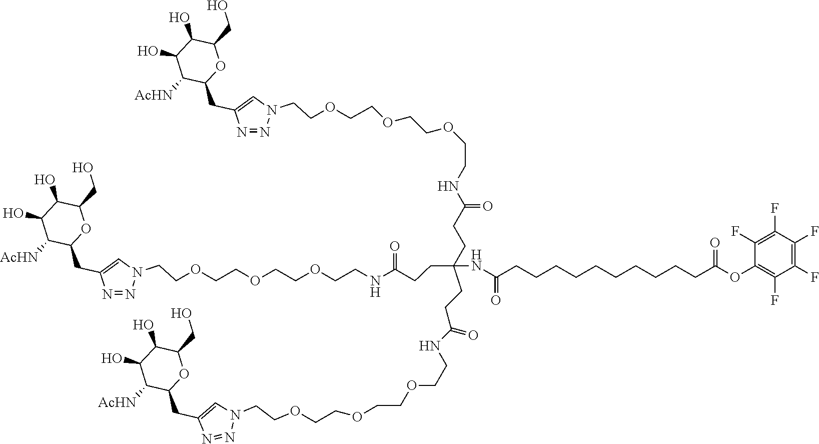 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1216 A | 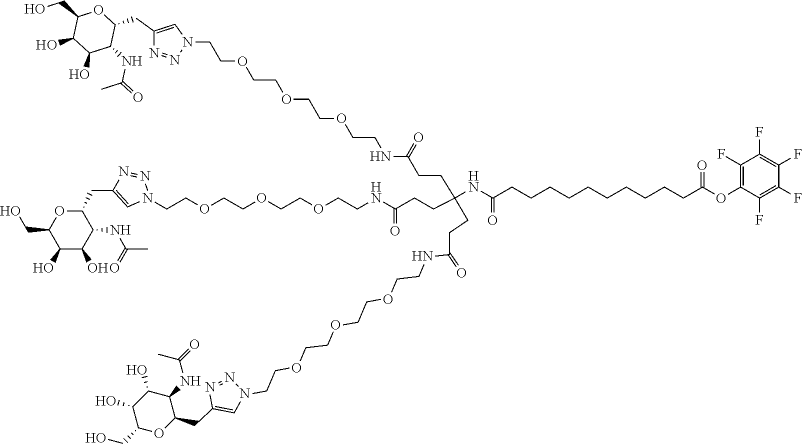 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1218 (I-143) | 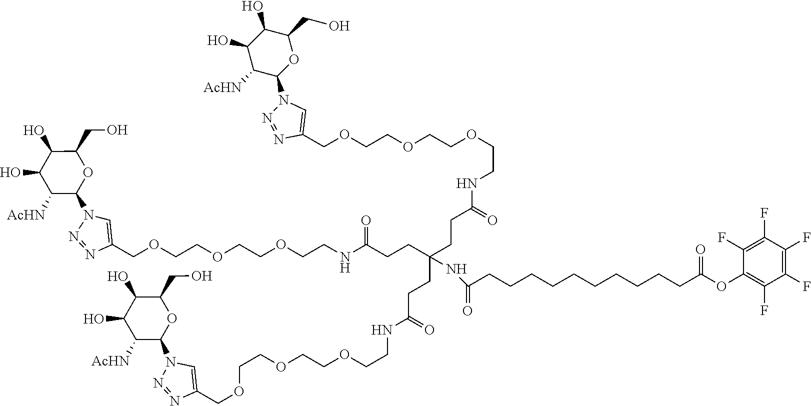 [SMILES: CC(=O)N[C@@H]1[C@@H](O)[C@@H](O)[C@@H](CO)O[C@H]1n1cc(COCCOCCOCCNC(=O)CCC(CCC(=O)NCCOCCOCCOCc2cn([C@@H]3O[C@H](CO)[C@H](O)[C@H](O)[C@H]3NC(C)=O)nn2)(CCC(=O)NCCOCCOCCOCc2cn([C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1219 (I-157) | 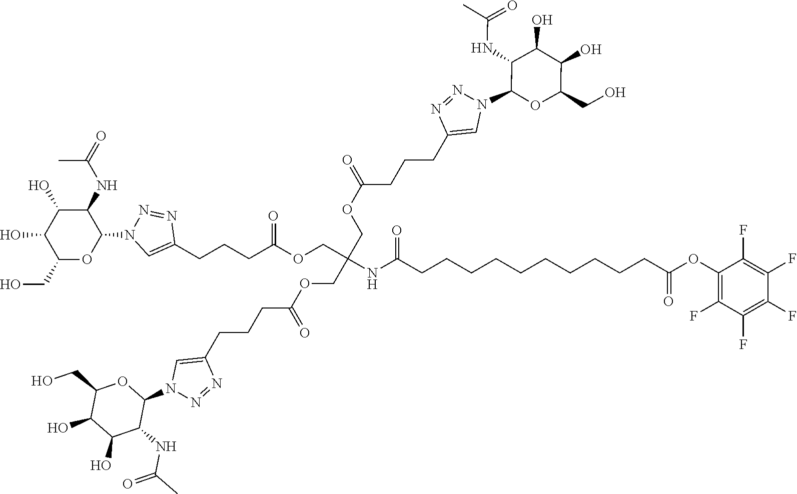 [SMILES: CC(=O)N[C@@H]1[C@@H](O)[C@@H](O)[C@@H](CO)O[C@H]1n1cc(CCCC(=O)OCC(COC(=O)CCCc2cn([C@@H]3O[C@H](CO)[C@H](O)[C@H](O)[C@H]3NC(C)=O)nn2)(COC(=O)CCCc2cn([C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1220 (I-158) | 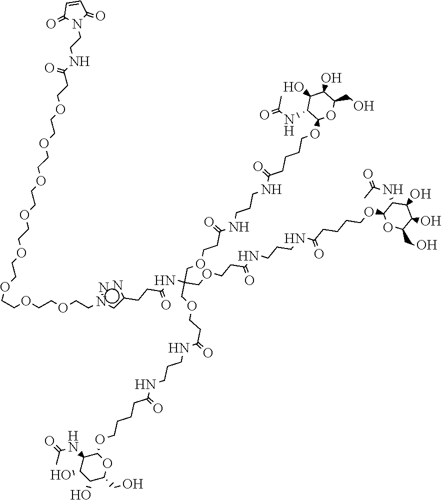 |
| 1221 (I-138) | 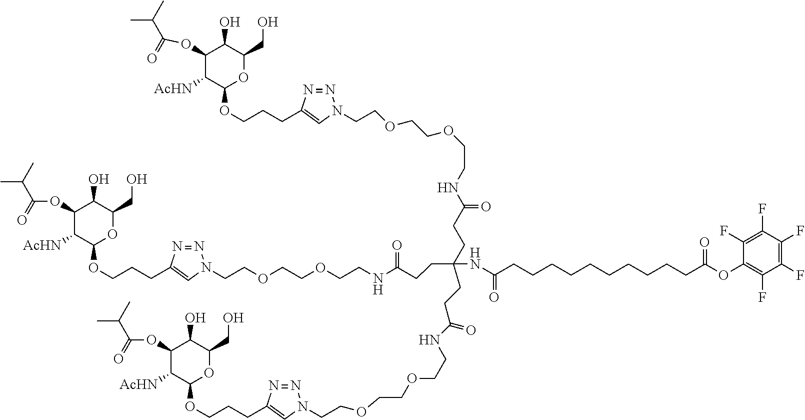 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](OC(=O)C(C)C)[C@H]4NC(C)=O)nn3)(CCC(=O)NCCOCCOCCn3cc(CCCO[C@@H]4O[C@H](CO)[C@H](O)[C@H](OC(=O)C(C)C)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1OC(=O)C(C)C] |
| 1222 (I-159) | 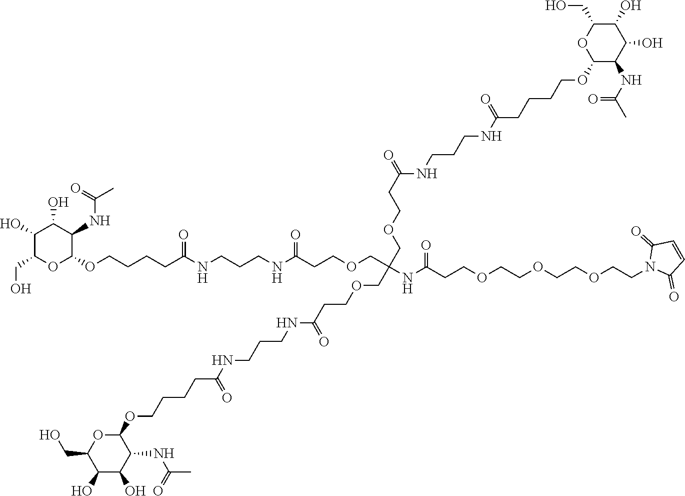 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCN2C(=O)C=CC2=O)O[C@H](CO)[C@H](O)[C@@H]1O] |
| 1223 (I-160) | 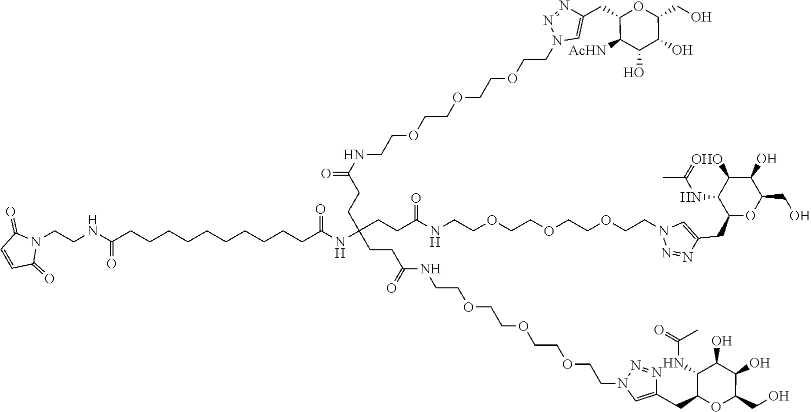 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1224 (I-161) | 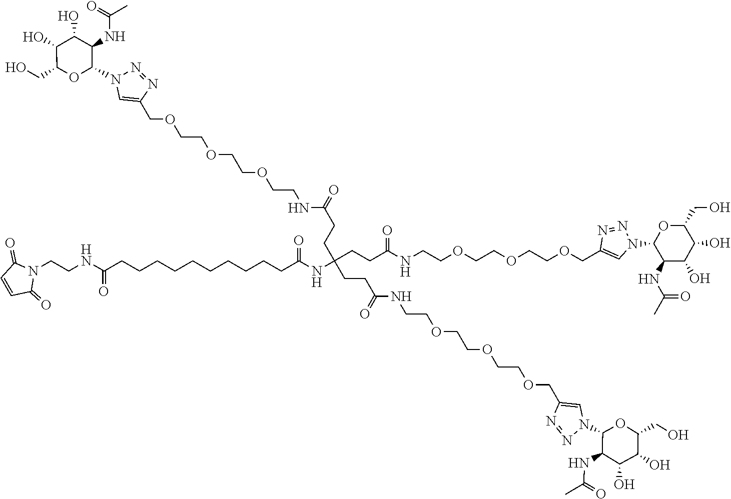 [SMILES: CC(=O)N[C@@H]1[C@@H](O)[C@@H](O)[C@@H](CO)O[C@H]1n1cc(COCCOCCOCCNC(=O)CCC(CCC(=O)NCCOCCOCCOCc2cn([C@@H]3O[C@H](CO)[C@H](O)[C@H](O)[C@H]3NC(C)=O)nn2)(CCC(=O)NCCOCCOCCOCc2cn([C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1225 (I-162) | 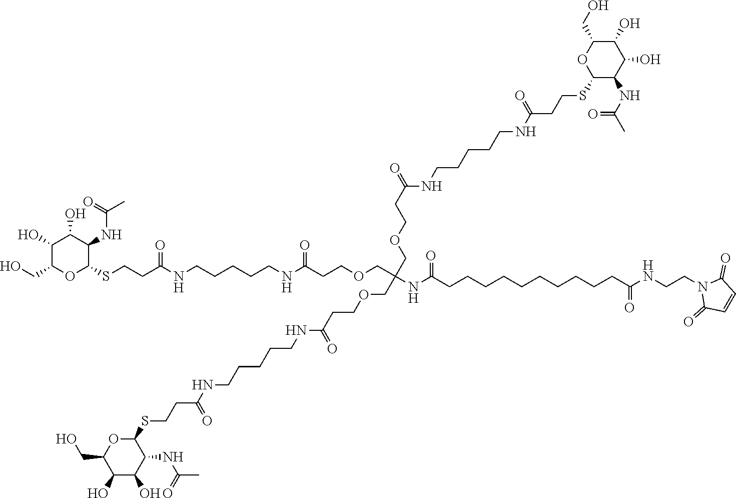 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)NCCN1C(=O)C=CC1=O] |
| 1225 A | 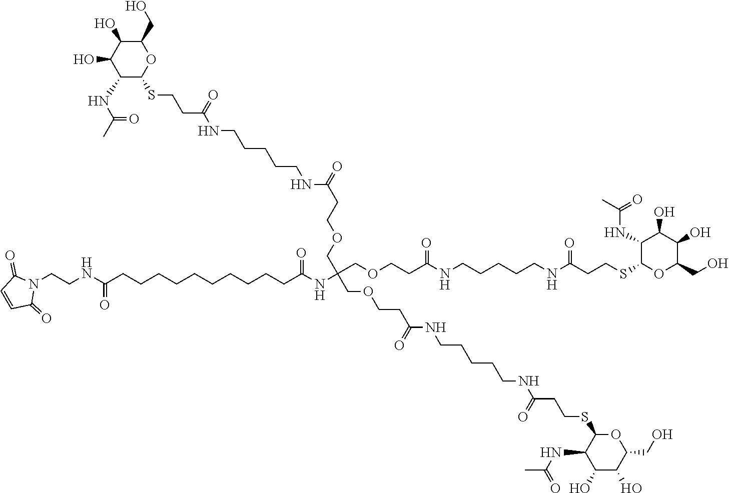 [SMILES: CC(=O)N[C@@H]1[C@@H](O)[C@@H](O)[C@@H](CO)O[C@@H]1SCCC(=O)NCCCCCNC(=O)CCOCC(COCCC(=O)NCCCCCNC(=O)CCS[C@H]1O[C@H](CO)[C@H](O)[C@H](O)[C@H]1NC(C)=O)(COCCC(=O)NCCCCCNC(=O)CCS[C@H]1O[C@H](CO)[C@H](O)[C@H](O)[C@H]1NC(C)=O)NC(=O)CCCCCCCCCCC(=O)NCCN1C(=O)C=CC1=O] |
| 1226 (I-163) | 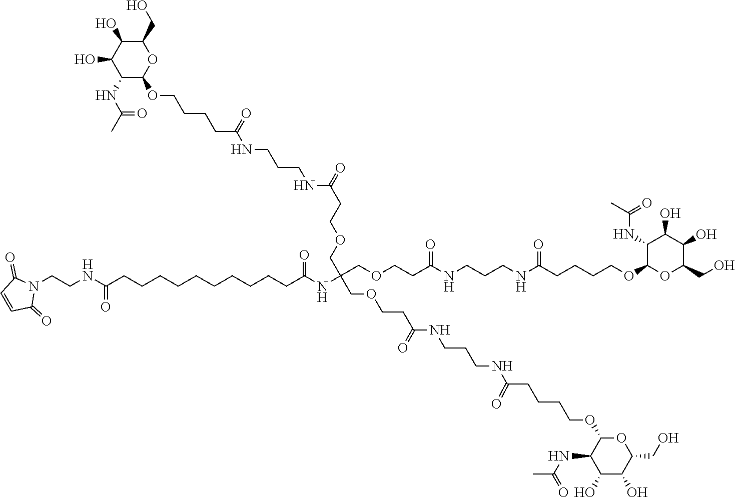 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| 1227 (I-170) | 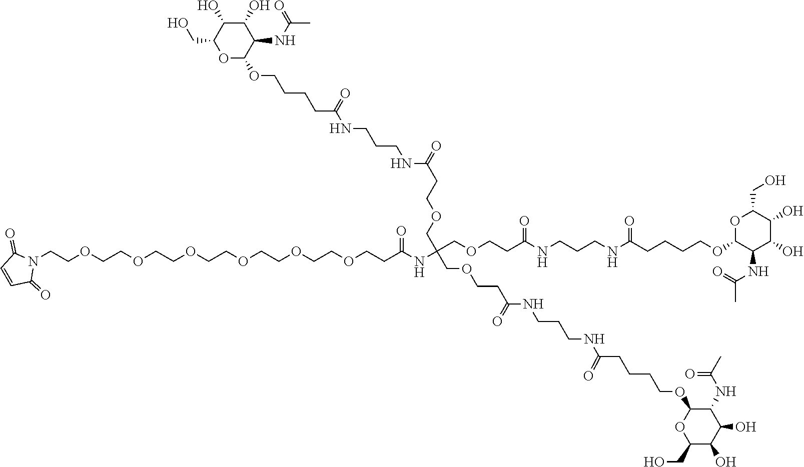 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCOCCOCCN2C(=O)C=CC2=O)O[C@H](CO)[C@H](O)[C@@H]1O] |
| 1228 | 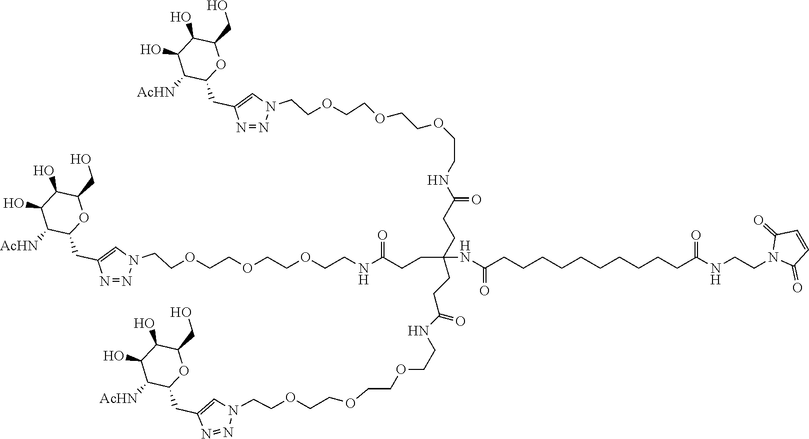 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1228 A | 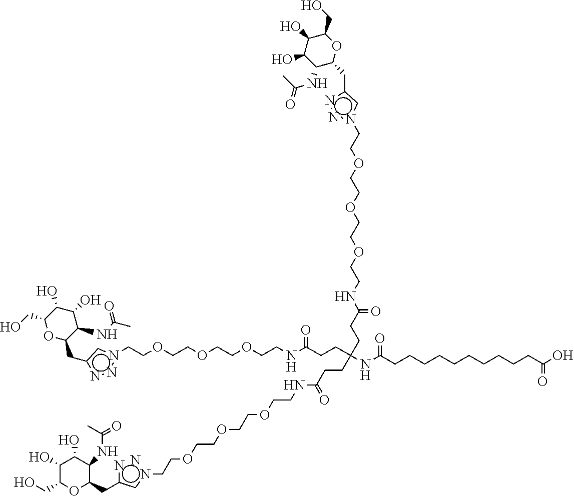 |
| 1228 B | 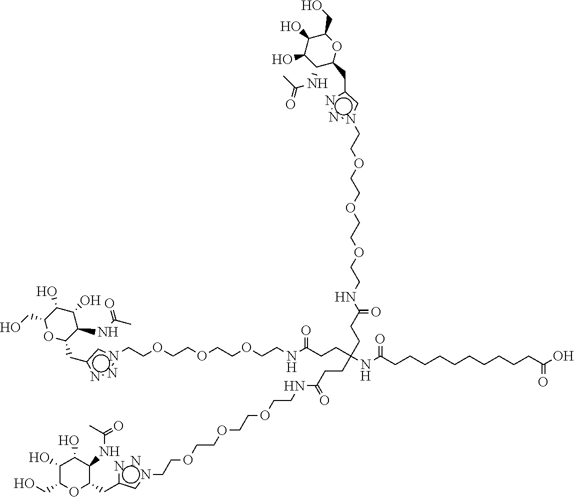 |
| 1229 | 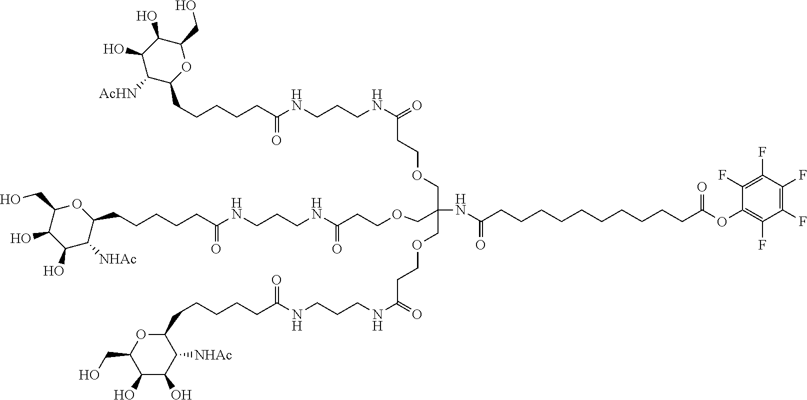 [SMILES: CC(=O)N[C@@H]1[C@@H](O)[C@@H](O)[C@@H](CO)O[C@H]1CCCCCC(=O)NCCCNC(=O)CCOCC(COCCC(=O)NCCCNC(=O)CCCCC[C@@H]1O[C@H](CO)[C@H](O)[C@H](O)[C@H]1NC(C)=O)(COCCC(=O)NCCCNC(=O)CCCCC[C@@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1230 | 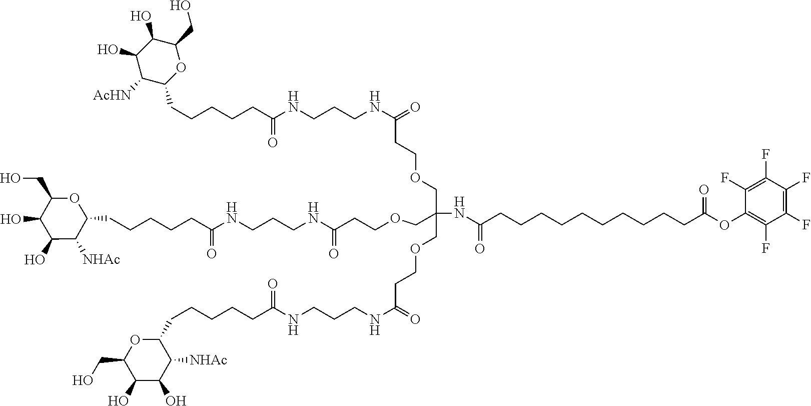 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1231 | 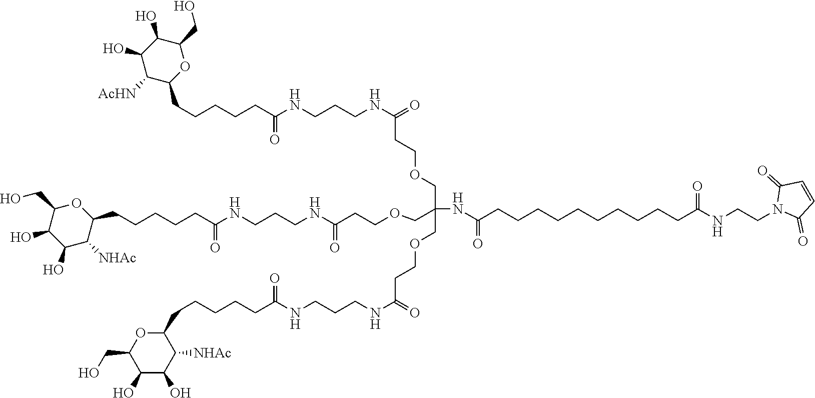 [SMILES: CC(=O)N[C@@H]1[C@@H](O)[C@@H](O)[C@@H](CO)O[C@H]1CCCCCC(=O)NCCCNC(=O)CCOCC(COCCC(=O)NCCCNC(=O)CCCCC[C@@H]1O[C@H](CO)[C@H](O)[C@H](O)[C@H]1NC(C)=O)(COCCC(=O)NCCCNC(=O)CCCCC[C@@H]1O[C@H](CO)[C@H](O)[C@H](O)[C@H]1NC(C)=O)NC(=O)CCCCCCCCCCC(=O)NCCN1C(=O)C=CC1=O] |
| 1232 | 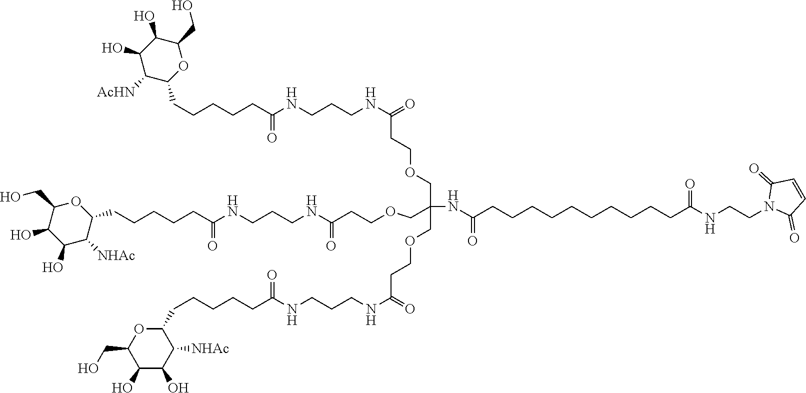 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)NCCN1C(=O)C=CC1=O] |
| 1232 A | 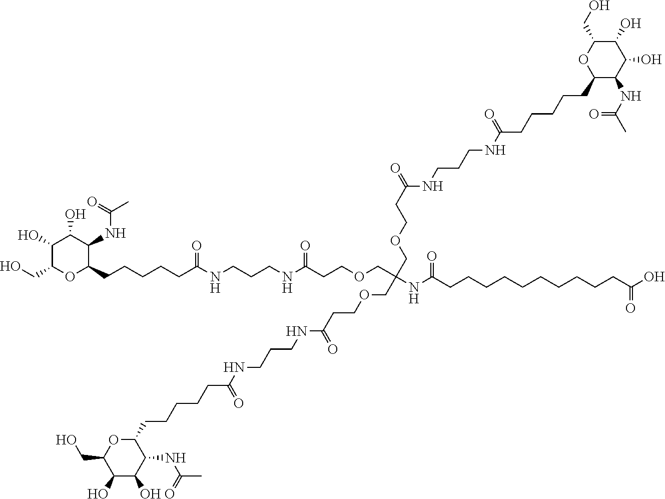 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)O] |
| 1233 | 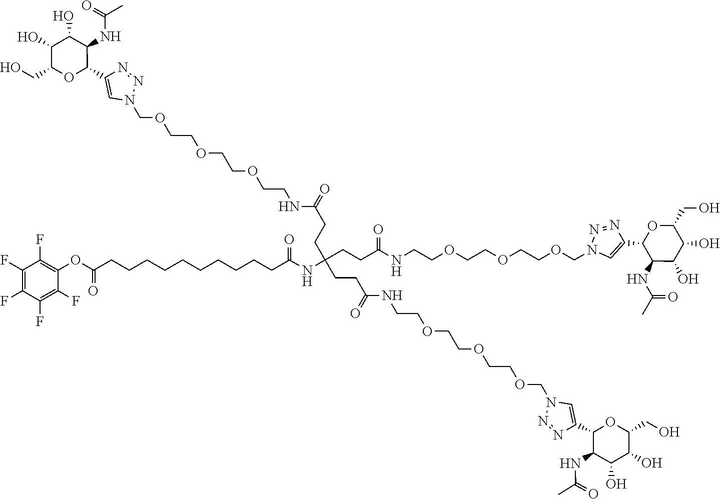 [SMILES: CC(=O)N[C@@H]1[C@@H](O)[C@@H](O)[C@@H](CO)O[C@H]1c1cn(COCCOCCOCCNC(=O)CCC(CCC(=O)NCCOCCOCCOCn2cc([C@@H]3O[C@H](CO)[C@H](O)[C@H](O)[C@H]3NC(C)=O)nn2)(CCC(=O)NCCOCCOCCOCn2cc([C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1233 B | 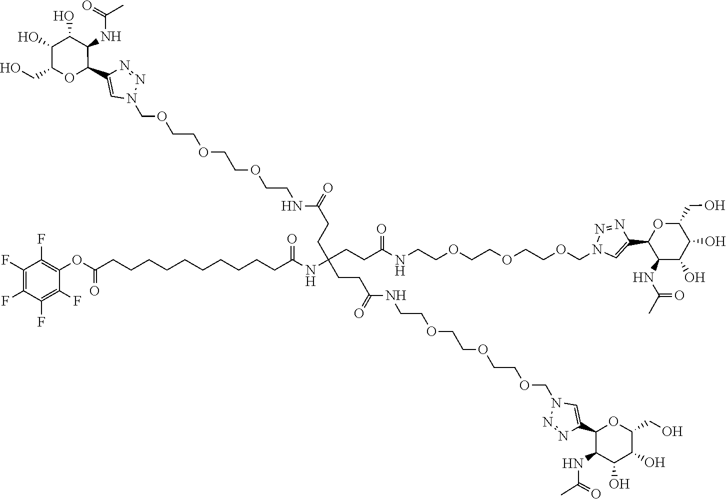 [SMILES: CC(=O)N[C@@H]1[C@@H](O)[C@@H](O)[C@@H](CO)O[C@@H]1c1cn(COCCOCCOCCNC(=O)CCC(CCC(=O)NCCOCCOCCOCn2cc([C@H]3O[C@H](CO)[C@H](O)[C@H](O)[C@H]3NC(C)=O)nn2)(CCC(=O)NCCOCCOCCOCn2cc([C@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1234 | 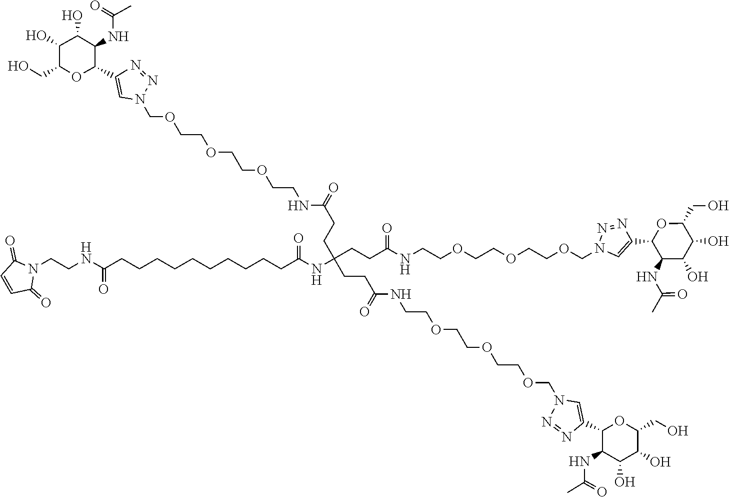 [SMILES: CC(=O)N[C@@H]1[C@@H](O)[C@@H](O)[C@@H](CO)O[C@H]1c1cn(COCCOCCOCCNC(=O)CCC(CCC(=O)NCCOCCOCCOCn2cc([C@@H]3O[C@H](CO)[C@H](O)[C@H](O)[C@H]3NC(C)=O)nn2)(CCC(=O)NCCOCCOCCOCn2cc([C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1234 B | 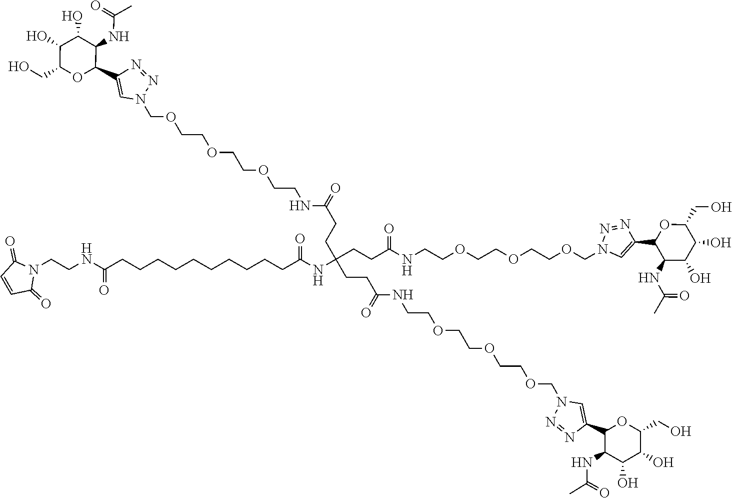 |
| 1243 | 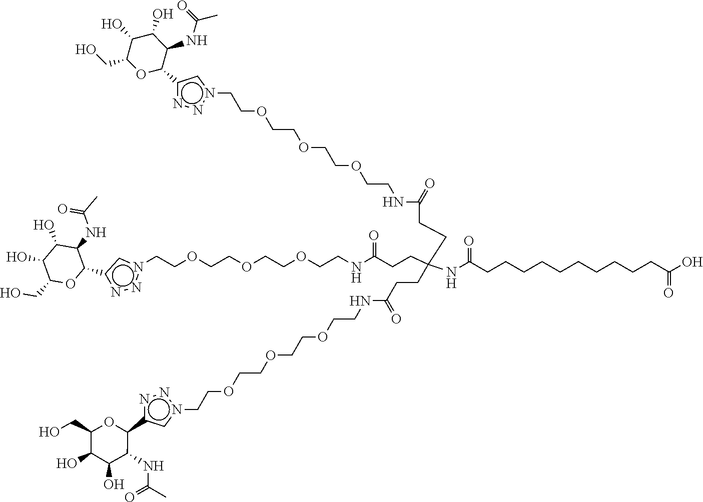 |
| 1244 | 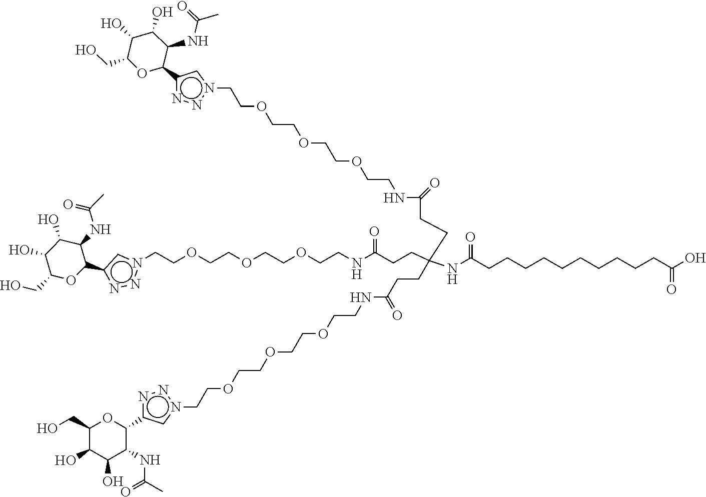 |
| 1245 25410 | 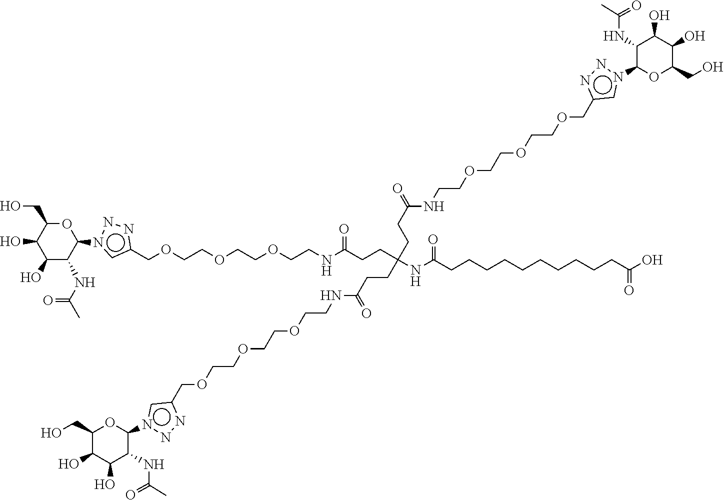 |
| 1246 | 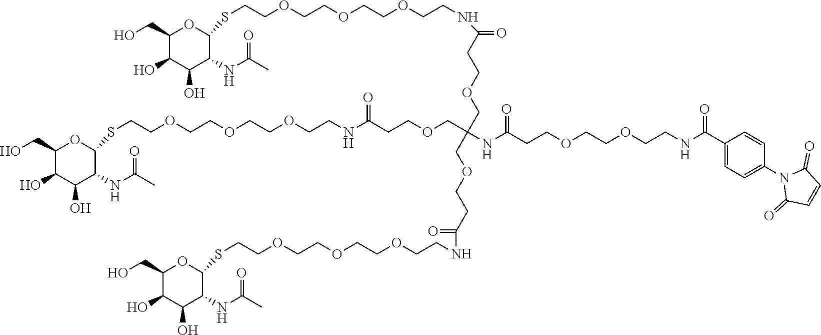 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCNC(=O)c1ccc(N2C(=O)C=CC2=O)cc1] |
| 1247 | 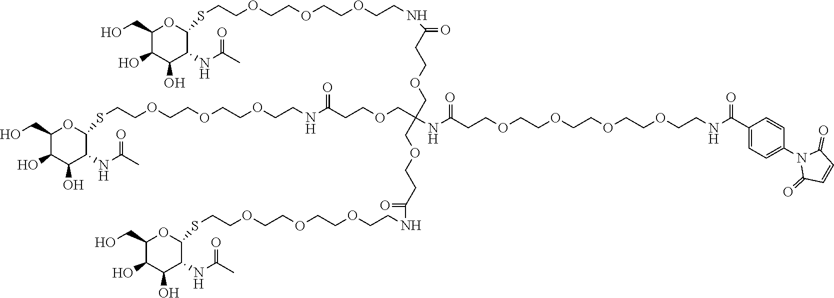 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1248 | 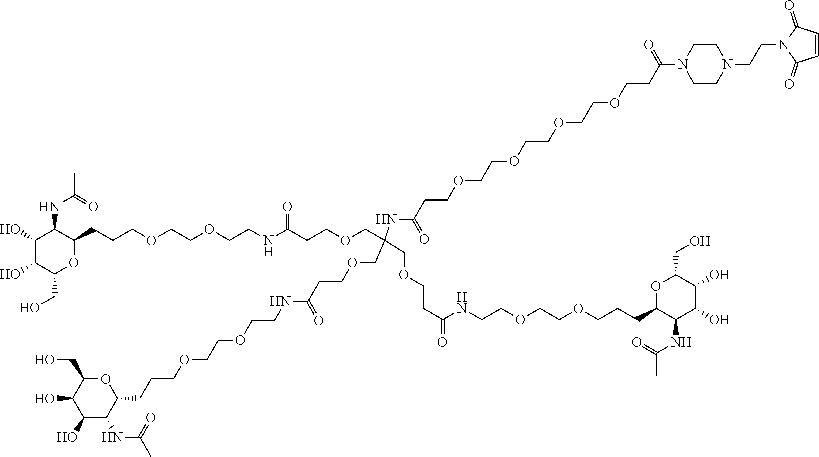 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1249 | 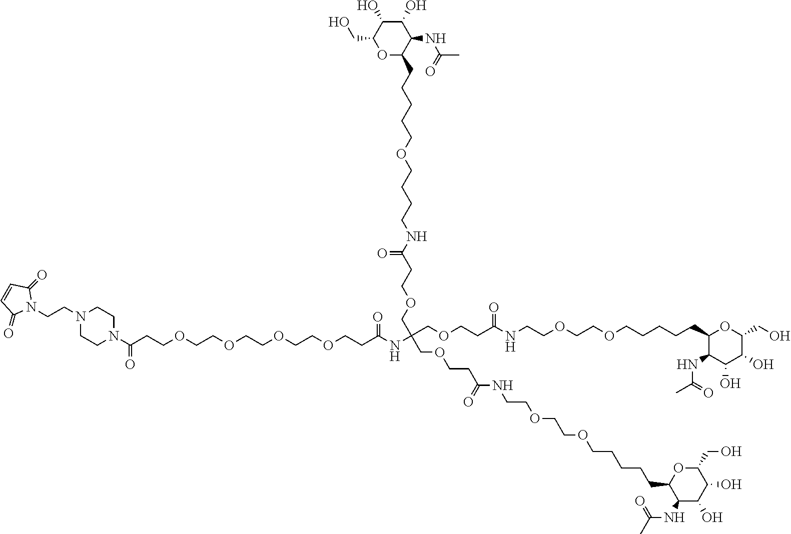 [SMILES: CC(=O)N[C@@H]1[C@@H](O)[C@@H](O)[C@@H](CO)O[C@@H]1CCCCCOCCCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1250 | 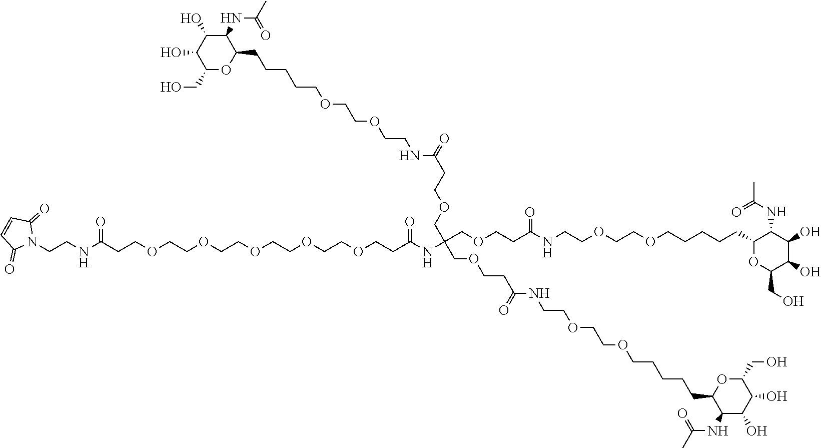 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCOCCC(=O)NCCN1C(=O)C=CC1=O] |
| 1251 | 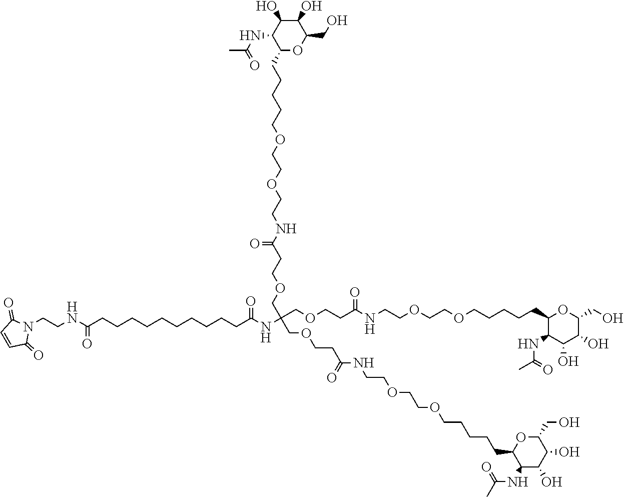 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCCCCCCCCCC(=O)NCCN1C(=O)C=CC1=O] |
| 1252 | 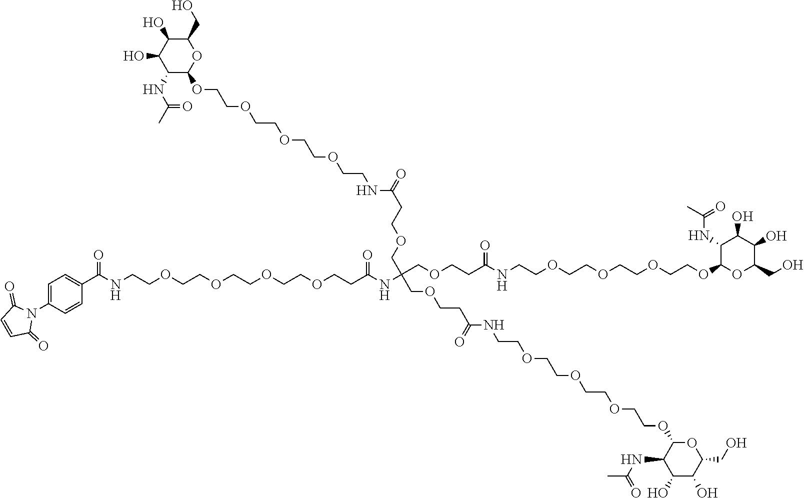 [SMILES: CC(=O)N[C@H]1[C@H](OCCOCCOCCOCCNC(=O)CCOCC(COCCC(=O)NCCOCCOCCOCCO[C@@H]2O[C@H](CO)[C@H](O)[C@H](O)[C@H]2NC(C)=O)(COCCC(=O)NCCOCCOCCOCCO[C@@H]2O[C@H](CO)[C@H](O)[C@H](O)[C@H]2NC(C)=O)NC(=O)CCOCCOCCOCCOCCNC(=O)c2ccc(N3C(=O)C=CC3=O)cc2)O[C@H](CO)[C@H](O)[C@@H]1O] |
| 1253 | 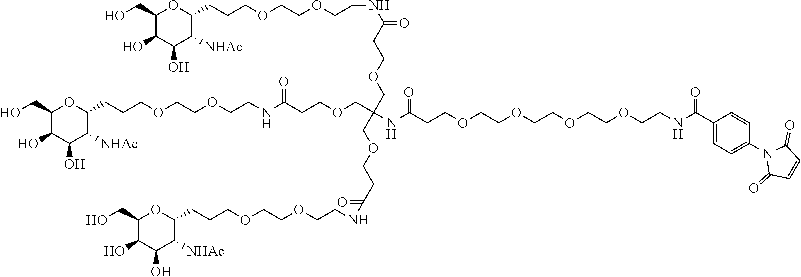 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1254 | 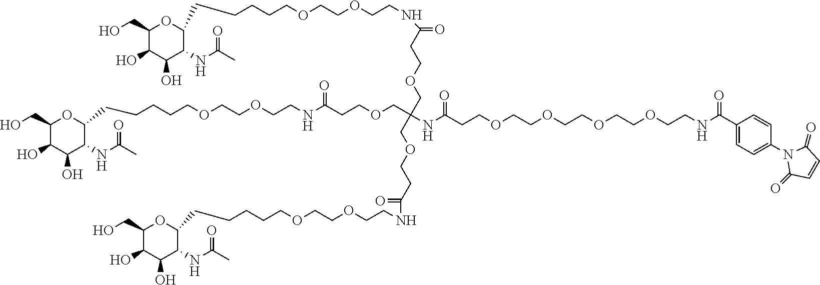 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1255 | 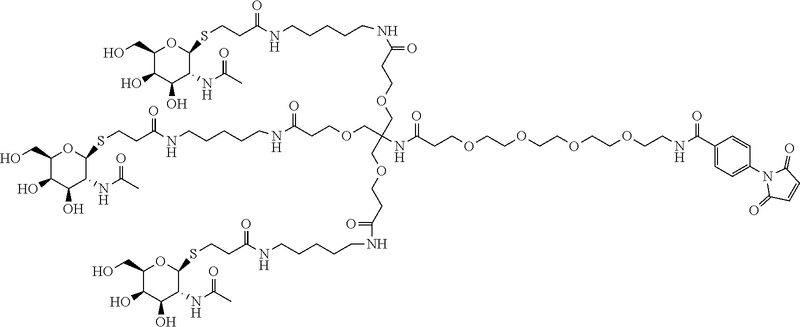 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1256 | 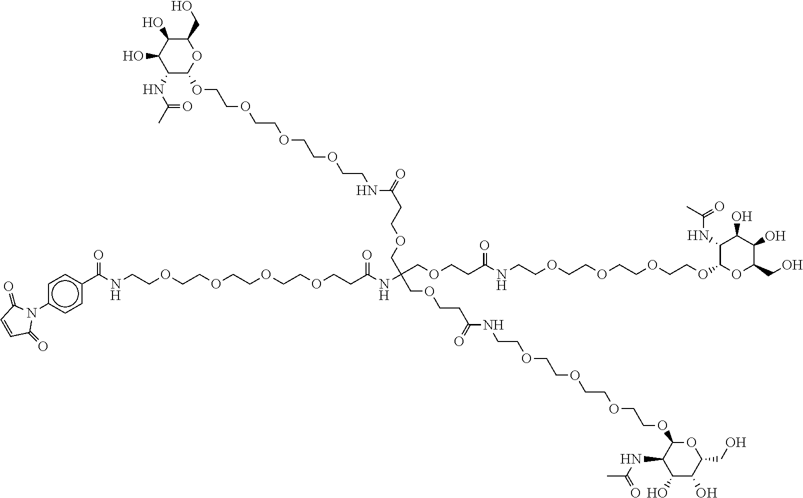 |
| 1257 | 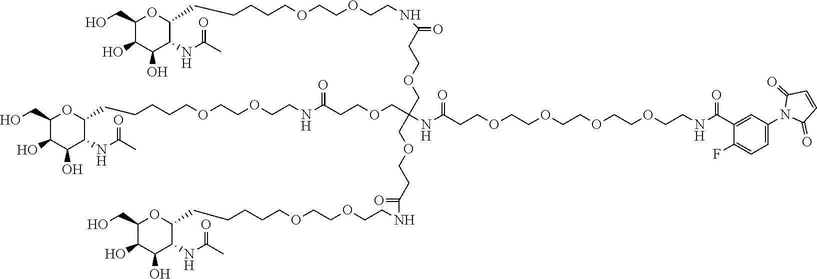 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1cc(N2C(=O)C=CC2=O)ccc1F] |
| 1258 | 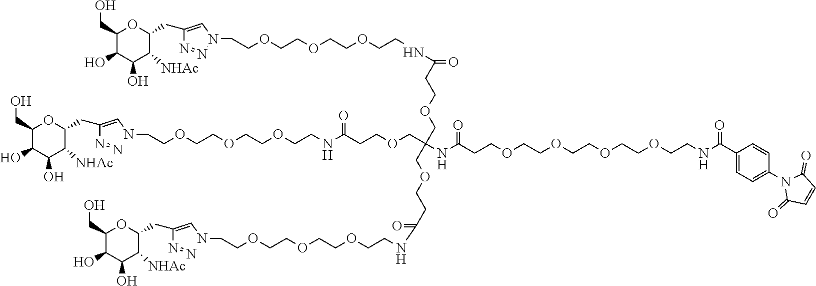 [SMILES: CC(=O)N[C@@H]1[C@@H](O)[C@@H](O)[C@@H](CO)O[C@@H]1Cc1cn(CCOCCOCCOCCNC(=O)CCOCC(COCCC(=O)NCCOCCOCCOCCn2cc(C[C@H]3O[C@H](CO)[C@H](O)[C@H](O)[C@H]3NC(C)=O)nn2)(COCCC(=O)NCCOCCOCCOCCn2cc(C[C@H]3O[C@H](CO)[C@H](O)[C@H](O)[C@H]3NC(C)=O)nn2)NC(=O)CCOCCOCCOCCOCCNC(=O)c2ccc(N3C(=O)C=CC3=O)cc2)nn1] |
| 1259 | 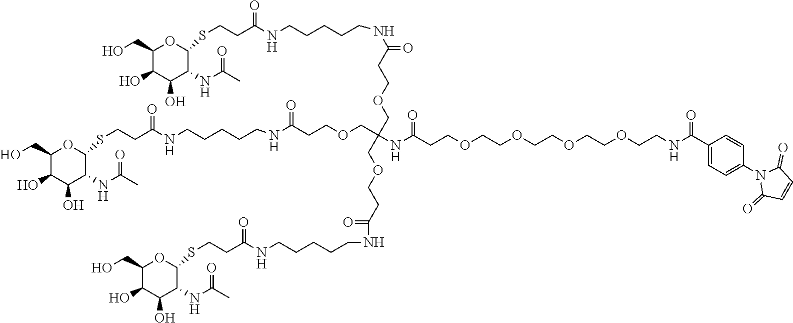 [SMILES: CC(=O)N[C@@H]1[C@@H](O)[C@@H](O)[C@@H](CO)O[C@@H]1SCCC(=O)NCCCCCNC(=O)CCOCC(COCCC(=O)NCCCCCNC(=O)CCS[C@H]1O[C@H](CO)[C@H](O)[C@H](O)[C@H]1NC(C)=O)(COCCC(=O)NCCCCCNC(=O)CCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1260 (32-11) | 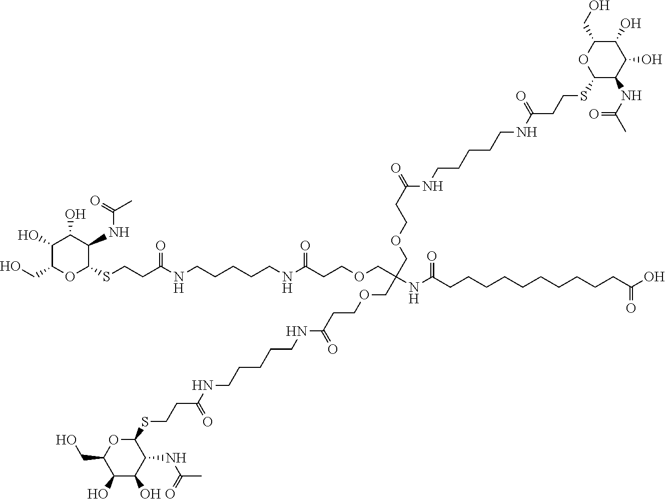 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)O] |

TABLE 16
Multivalent ASGPR binding compounds having chemoselective ligation group and X
group of formula (Ic)

| # | Structure |
|---|---|
| 1301 (I-110) | 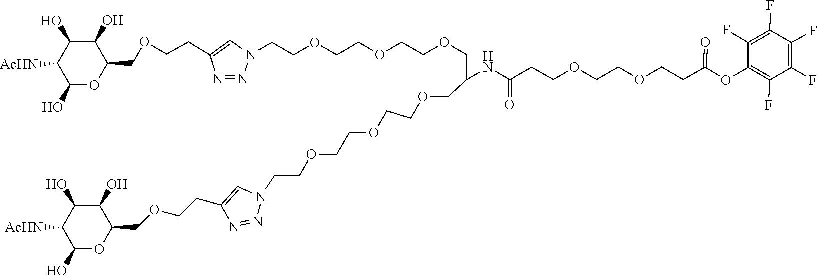 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |

Trimeric ligands

| | |
|---|---|
| 1302 (I- 108) | 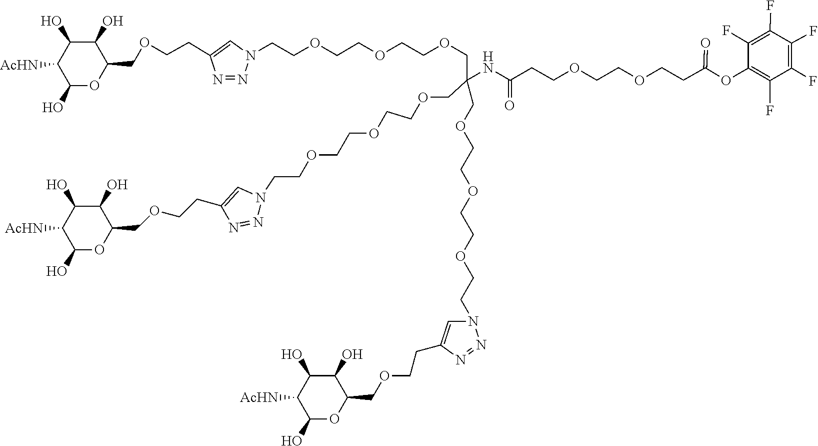 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 1303 (I- 136) | 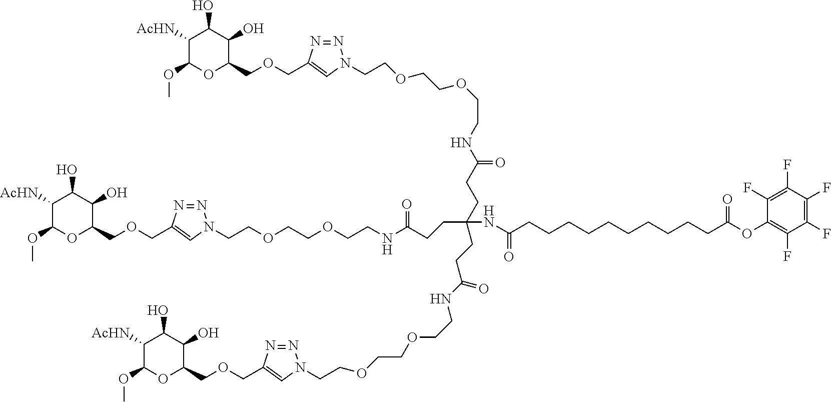 [SMILES: CO[C@@H]1O[C@H](COCc2cn(CCOCCOCCNC(=O)CCC(CCC(=O)NCCOCCOCCn3cc(COC[C@H]4O[C@@H](OC)[C@H](NC(C)=O)[C@@H](O)[C@H]4O)nn3)(CCC(=O)NCCOCCOCCn3cc(COC[C@H]4O[C@@H](OC)[C@H](NC(C)=O)[C@@H](O)[C@H]4O)nn3)NC(=O)CCCCCCCCCCC(=O)Oc3c(F)c(F)c(F)c(F)c3F)nn2)[C@H](O)[C@H](O)[C@H]1NC(C)=O] |
| 1304 (I- 152) | 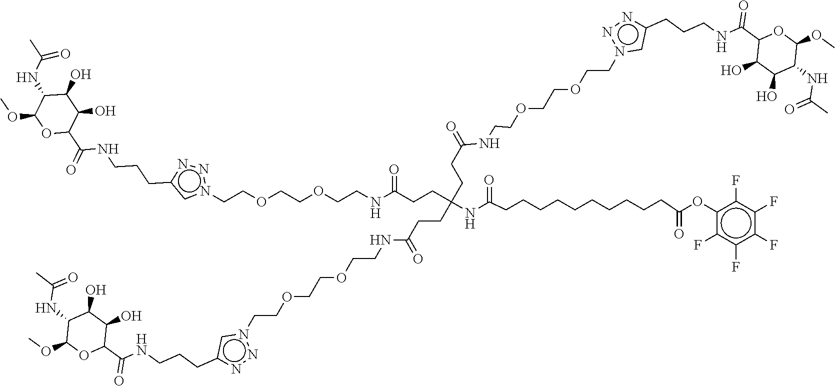 |

TABLE 17
Multivalent ASGPR binding compounds having chemoselective ligation group and X
group of formula (Id)

| # | Structure |
|---|---|
| 1401 (I- 153) | 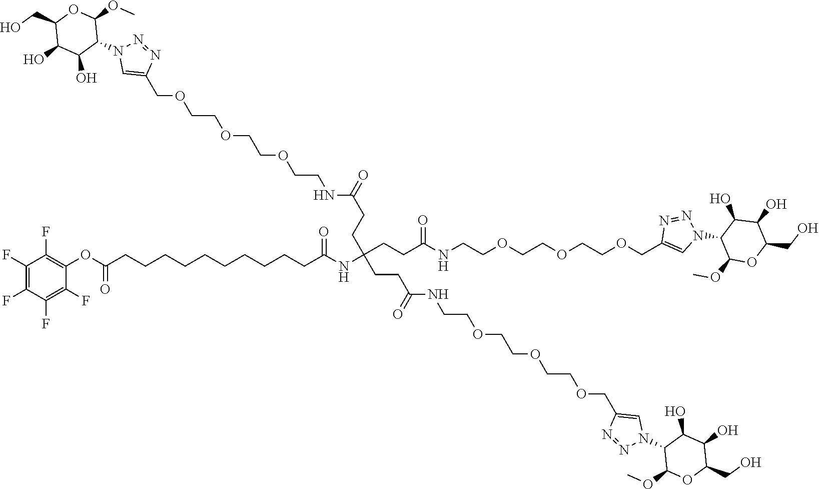 [SMILES: CO[C@@H]1O[C@H](CO)[C@H](O)[C@H](O)[C@H]1n1cc(COCCOCCOCCNC(=O)CCC(CCC(=O)NCCOCCOCCOCc2cn([C@H]3[C@H](OC)O[C@H](CO)[C@H](O)[C@@H]3O)nn2)(CCC(=O)NCCOCCOCCOCc2cn([C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |
| 1402 (I- 154) | 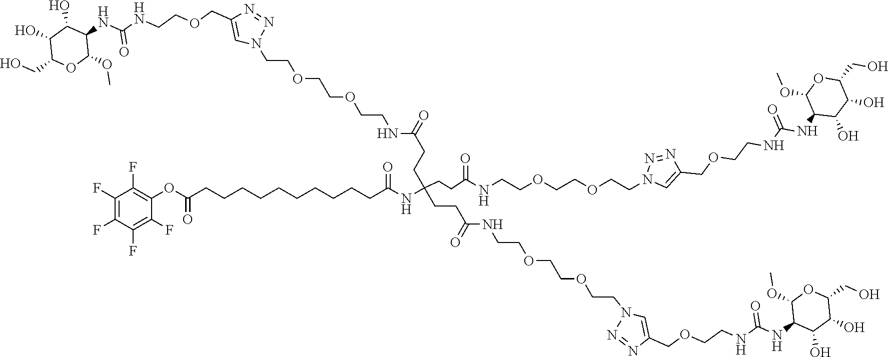 [SMILES: CO[C@@H]1O[C@H](CO)[C@H](O)[C@H](O)[C@H]1NC(=O)NCCOCc1cn(CCOCCOCCNC(=O)CCC(CCC(=O)NCCOCCOCCn2cc(COCCNC(=O)N[C@H]3[C@H](OC)O[C@H](CO)[C@H](O)[C@@H]3O)nn2)(CCC(=O)NCCOCCOCCn2cc(COCCNC(=O)N[C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |
| 1403 (I- 155) | 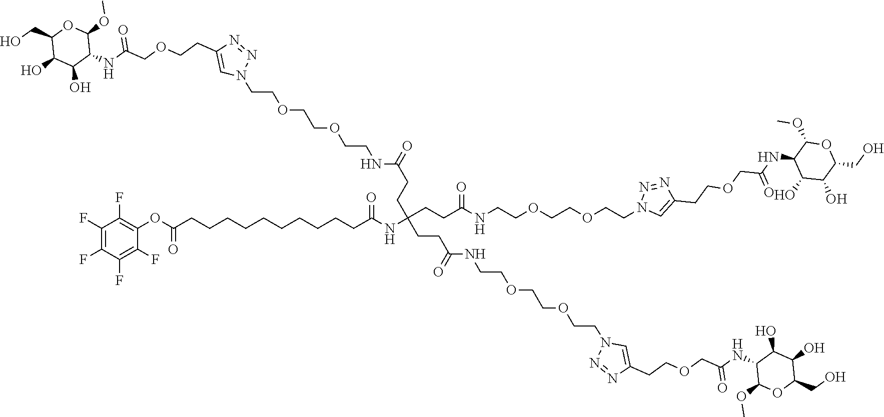 [SMILES: CO[C@@H]1O[C@H](CO)[C@H](O)[C@H](O)[C@H]1NC(=O)COCCc1cn(CCOCCOCCNC(=O)CCC(CCC(=O)NCCOCCOCCn2cc(CCOCC(=O)N[C@H]3[C@H](OC)O[C@H](CO)[C@H](O)[C@@H]3O)nn2)(CCC(=O)NCCOCCOCCn2cc(CCOCC(=O)N[C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |

The present disclosure is meant to encompass stereoisomers of any one of the compounds described herein. In some instance, the compound includes an enantiomer of the D- N-acetylgalactosamine (GalNAc), or an analog or derivative of GalNAc.

### Other Exemplary Compounds

Table 18 illustrates exemplary ASGPR binding compounds of this disclosure that include a binding moiety, or a precursor thereof.

TABLE 18
Multivalent ASGPR binding compounds having protein targeting group

| # | Structure |
|---|---|
| 1404 (I- 156) | 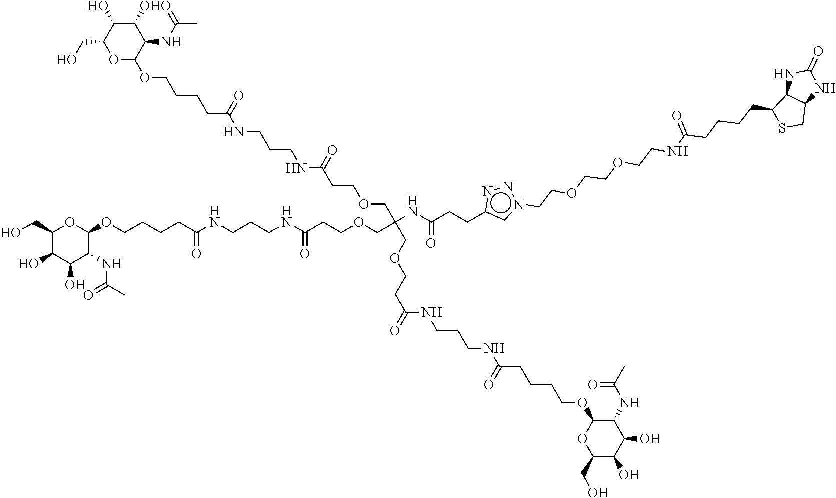 |
| 1405 (I- 139) | 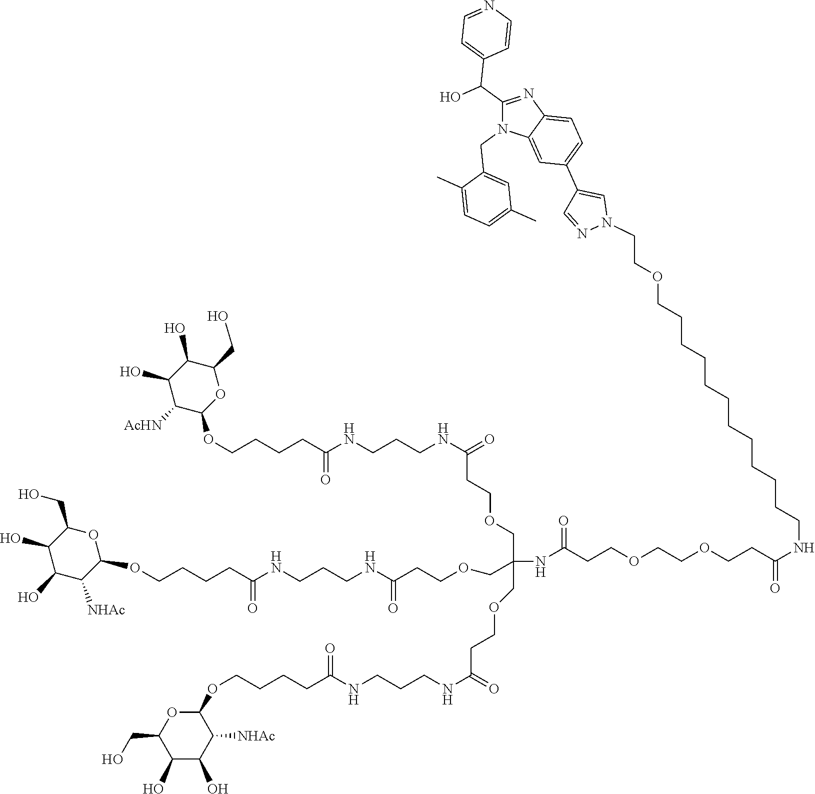 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCC(=O)NCCCCCCCCCCCCOCCn2cc(-c3ccc4nc(C(O)c5ccncc5)n(Cc5cc(C)ccc5C)c4c3)cn2)O[C@H](CO)[C@H](O)[C@@H]1O] |
| 1406 (I- 142) | 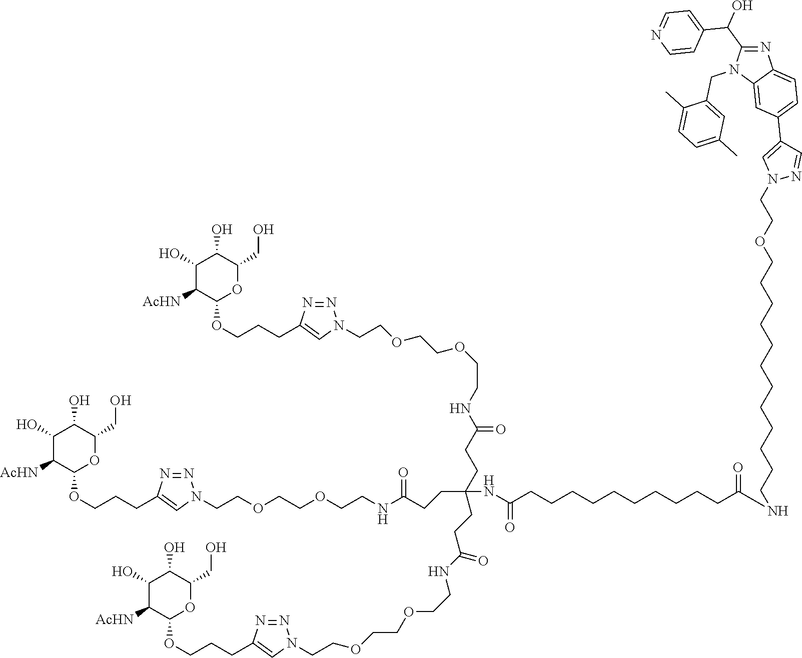 [SMILES: CC(=O)N[C@@H]1[C@@H](OCCCc2cn(CCOCCOCCNC(=O)CCC(CCC(=O)NCCOCCOCCn3cc(CCCO[C@H]4O[C@@H](CO)[C@@H](O)[C@@H](O)[C@@H]4NC(C)=O)nn3)(CCC(=O)NCCOCCOCCn3cc(CCCO[C@H]4O[C@@H](CO)[C@@H](O)[C@@H](O)[C@@H]4NC(C)=O)nn3)NC(=O)CCCCCCCCCCC(=O)NCCCCCCCCCCCCOCCn3cc(-c4ccc5nc(C(O)c6ccncc6)n(Cc6cc(C)ccc6C)c5c4)cn3)nn2)O[C@@H](CO)[C@@H](O)[C@H]1O] |

Table 19 illustrates exemplary trivalent ASGPR binding intermediate compounds of this disclosure including X groups of formula (Ie).

TABLE 19
Multivalent ASGPR binding compounds including X groups of formula (Ie)

| # | Structure |
|---|---|
| 1901 (I- 171) | 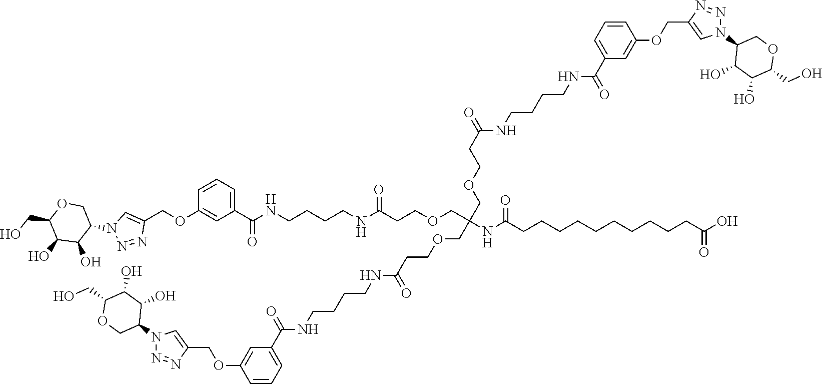 [SMILES: O=C(O)CCCCCCCCCCC(=O)NC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)COCCC(=O)NCCCCNC(=O)c1cccc(OCc2cn([C@H]3CO[C@H](CO)[C@H](O)[C@@H]3O)nn2)c1] |
| 1902 (I- 172) | 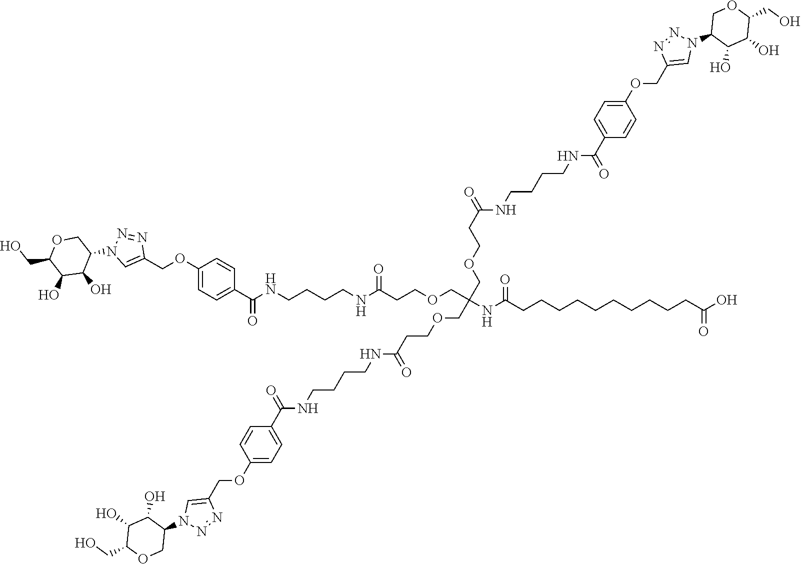 [SMILES: O=C(O)CCCCCCCCCCC(=O)NC(COCCC(=O)NCCCCNC(=O)c1ccc(OCc2cn([C@H]3CO[C@H](CO)[C@H](O)[C@@H]3O)nn2)cc1)(COCCC(=O)NCCCCNC(=O)c1ccc(OCc2cn([C@H]3CO[C@H](CO)[C@H](O)[C@@H]3O)nn2)cc1)COCCC(=O)NCCCCNC(=O)c1ccc(OCc2cn([C@H]3CO[C@H](CO)[C@H](O)[C@@H]3O)nn2)cc1] |
| 1904 | 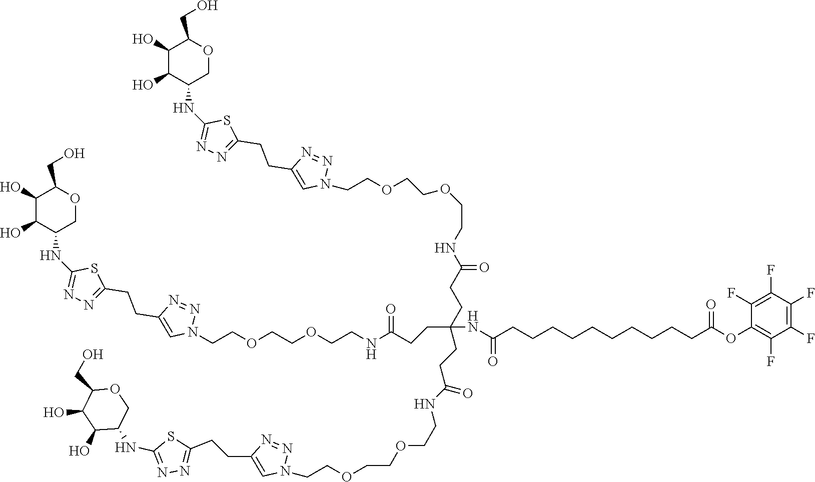 [SMILES: O=C(CCC(CCC(=O)NCCOCCOCCn1cc(CCc2nnc(N[C@H]3CO[C@H](CO)[C@H](O)[C@@H]3O)s2)nn1)(CCC(=O)NCCOCCOCCn1cc(CCc2nnc(N[C@H]3CO[C@H](CO)[C@H](O)[C@@H]3O)s2)nn1)NC(=O)CCCCCCCCCCC(=O)Oc1c(F)c(F)c(F)c(F)c1F)NCCOCCOCCn1cc(CCc2nnc(N[C@H]3CO[C@H](CO)[C@H](O)[C@@H]3O)s2)nn1] |
| 1905 | 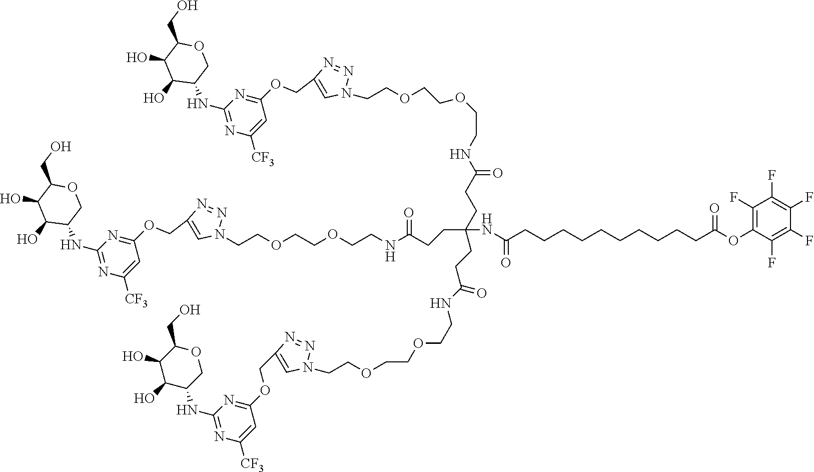 [SMILES: O=C(CCC(CCC(=O)NCCOCCOCCn1cc(COc2cc(C(F)(F)F)nc(N[C@H]3CO[C@H](CO)[C@H](O)[C@@H]3O)n2)nn1)(CCC(=O)NCCOCCOCCn1cc(COc2cc(C(F)(F)F)nc(N[C@H]3CO[C@H](CO)[C@H](O)[C@@H]3O)n2)nn1)NC(=O)CCCCCCCCCCC(=O)Oc1c(F)c(F)c(F)c(F)c1F)NCCOCCOCCn1cc(COc2cc(C(F)(F)F)nc(N[C@H]3CO[C@H](CO)[C@H](O)[C@@H]3O)n2)nn1] |
| 1905 A | 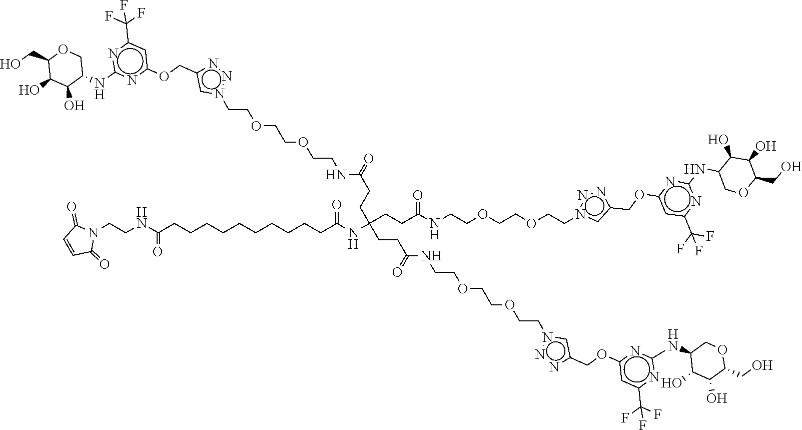 |
| 1906 | 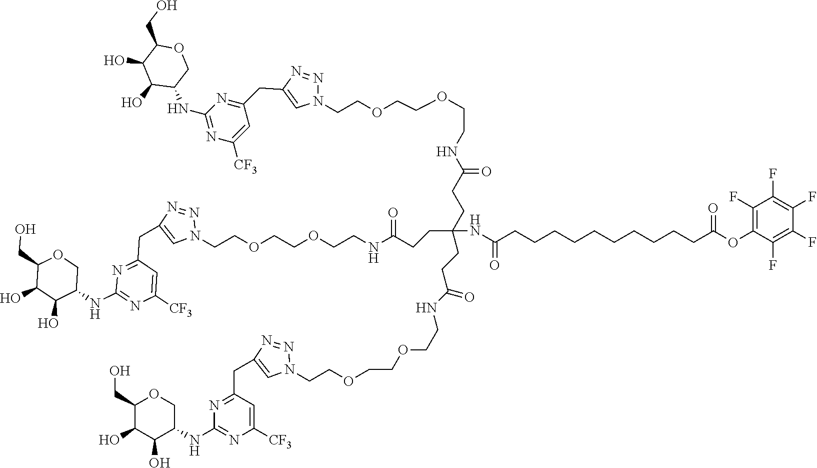 [SMILES: O=C(CCC(CCC(=O)NCCOCCOCCn1cc(Cc2cc(C(F)(F)F)nc(N[C@H]3CO[C@H](CO)[C@H](O)[C@@H]3O)n2)nn1)(CCC(=O)NCCOCCOCCn1cc(Cc2cc(C(F)(F)F)nc(N[C@H]3CO[C@H](CO)[C@H](O)[C@@H]3O)n2)nn1)NC(=O)CCCCCCCCCCC(=O)Oc1c(F)c(F)c(F)c(F)c1F)NCCOCCOCCn1cc(Cc2cc(C(F)(F)F)nc(N[C@H]3CO[C@H](CO)[C@H](O)[C@@H]3O)n2)nn1] |
| 1907 | 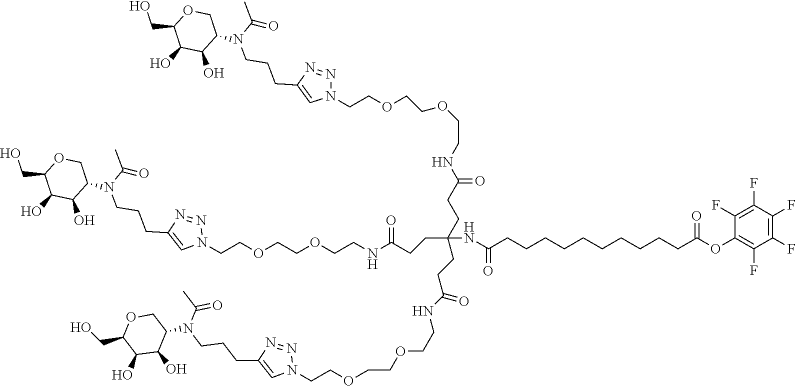 [SMILES: CC(=O)N(CCCc1cn(CCOCCOCCNC(=O)CCC(CCC(=O)NCCOCCOCCn2cc(CCCN(C(C)=O)[C@H]3CO[C@H](CO)[C@H](O)[C@@H]3O)nn2)(CCC(=O)NCCOCCOCCn2cc(CCCN(C(C)=O)[C@H]3CO[C@H](CO)[C@H](O)[C@@H]3O)nn2)NC(=O)CCCCCCCCCCC(=O)Oc2c(F)c(F)c(F)c(F)c2F)nn1)[C@H]1CO[C@H](CO)[C@H](O)[C@@H]1O] |
| 1908 | 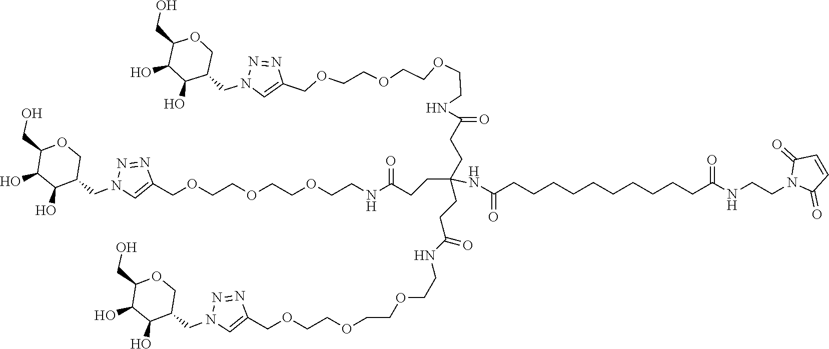 [SMILES: O=C(CCCCCCCCCCC(=O)NC(CCC(=O)NCCOCCOCCOCc1cn(C[C@H]2CO[C@H](CO)[C@H](O)[C@@H]2O)nn1)(CCC(=O)NCCOCCOCCOCc1cn(C[C@H]2CO[C@H](CO)[C@H](O)[C@@H]2O)nn1)CCC(=O)NCCOCCOCCOCc1cn(C[C@H]2CO[C@H](CO)[C@H](O)[C@@H]2O)nn1)NCCN1C(=O)C=CC1=O] |
| 1909 | 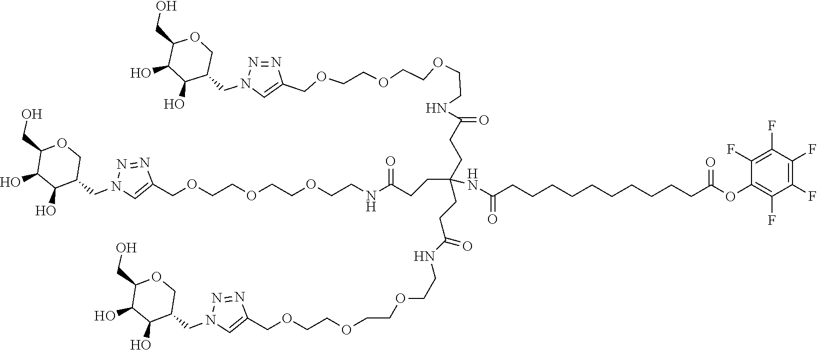 [SMILES: O=C(CCC(CCC(=O)NCCOCCOCCOCc1cn(C[C@H]2CO[C@H](CO)[C@H](O)[C@@H]2O)nn1)(CCC(=O)NCCOCCOCCOCc1cn(C[C@H]2CO[C@H](CO)[C@H](O)[C@@H]2O)nn1)NC(=O)CCCCCCCCCCC(=O)Oc1c(F)c(F)c(F)c(F)c1F)NCCOCCOCCOCc1cn(C[C@H]2CO[C@H](CO)[C@H](O)[C@@H]2O)nn1] |
| 1910 | 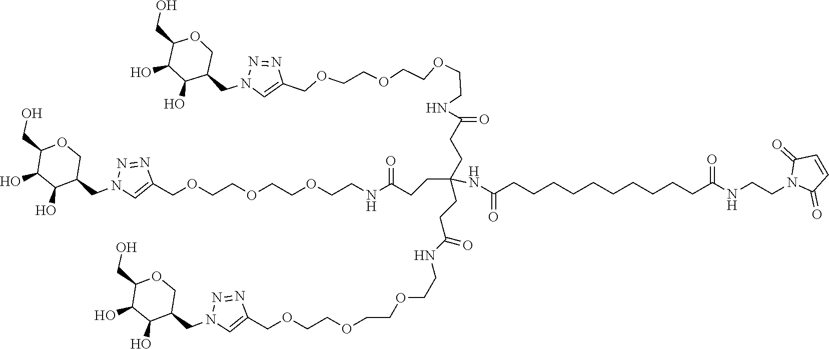 [SMILES: O=C(CCCCCCCCCCC(=O)NC(CCC(=O)NCCOCCOCCOCc1cn(C[C@@H]2CO[C@H](CO)[C@H](O)[C@@H]2O)nn1)(CCC(=O)NCCOCCOCCOCc1cn(C[C@@H]2CO[C@H](CO)[C@H](O)[C@@H]2O)nn1)CCC(=O)NCCOCCOCCOCc1cn(C[C@@H]2CO[C@H](CO)[C@H](O)[C@@H]2O)nn1)NCCN1C(=O)C=CC1=O] |
| 1911 | 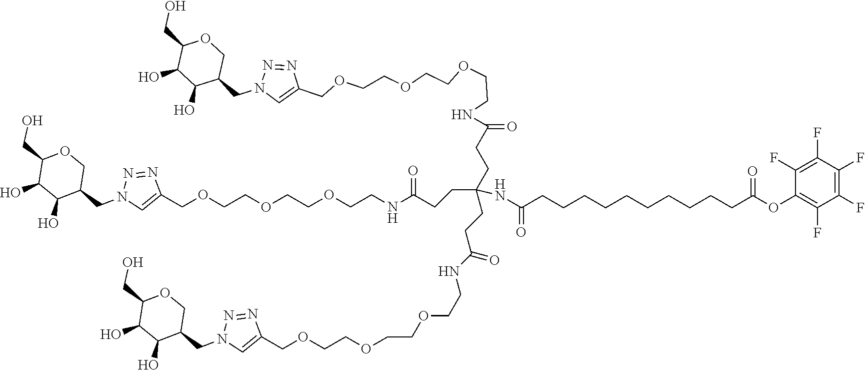 [SMILES: O=C(CCC(CCC(=O)NCCOCCOCCOCc1cn(C[C@@H]2CO[C@H](CO)[C@H](O)[C@@H]2O)nn1)(CCC(=O)NCCOCCOCCOCc1cn(C[C@@H]2CO[C@H](CO)[C@H](O)[C@@H]2O)nn1)NC(=O)CCCCCCCCCCC(=O)Oc1c(F)c(F)c(F)c(F)c1F)NCCOCCOCCOCc1cn(C[C@@H]2CO[C@H](CO)[C@H](O)[C@@H]2O)nn1] |
| 1912 | 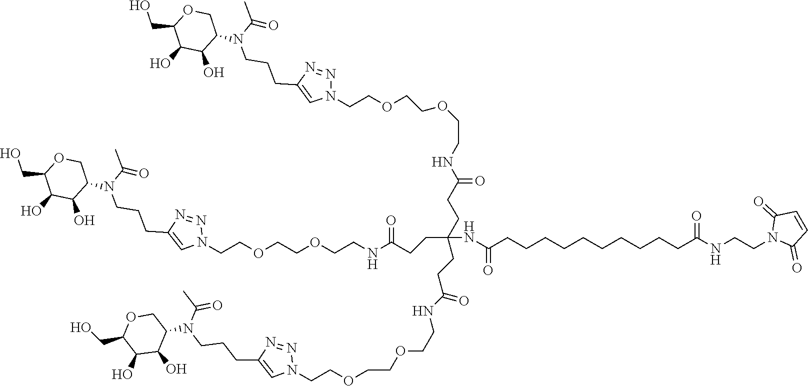 [SMILES: CC(=O)N(CCCc1cn(CCOCCOCCNC(=O)CCC(CCC(=O)NCCOCCOCCn2cc(CCCN(C(C)=O)[C@H]3CO[C@H](CO)[C@H](O)[C@@H]3O)nn2)(CCC(=O)NCCOCCOCCn2cc(CCCN(C(C)=O)[C@H]3CO[C@H](CO)[C@H](O)[C@@H]3O)nn2)NC(=O)CCCCCCCCCCC(=O)NCCN2C(=O)C=CC2=O)nn1)[C@H]1CO[C@H](CO)[C@H](O)[C@@H]1O] |
| 1913 | 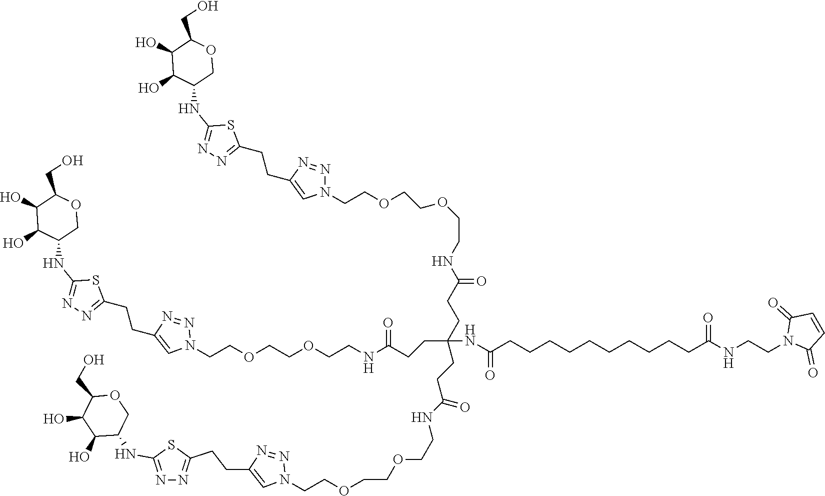 [SMILES: O=C(CCCCCCCCCCC(=O)NC(CCC(=O)NCCOCCOCCn1cc(CCc2nnc(N[C@H]3CO[C@H](CO)[C@H](O)[C@@H]3O)s2)nn1)(CCC(=O)NCCOCCOCCn1cc(CCc2nnc(N[C@H]3CO[C@H](CO)[C@H](O)[C@@H]3O)s2)nn1)CCC(=O)NCCOCCOCCn1cc(CCc2nnc(N[C@H]3CO[C@H](CO)[C@H](O)[C@@H]3O)s2)nn1)NCCN1C(=O)C=CC1=O] |
| 1914 | 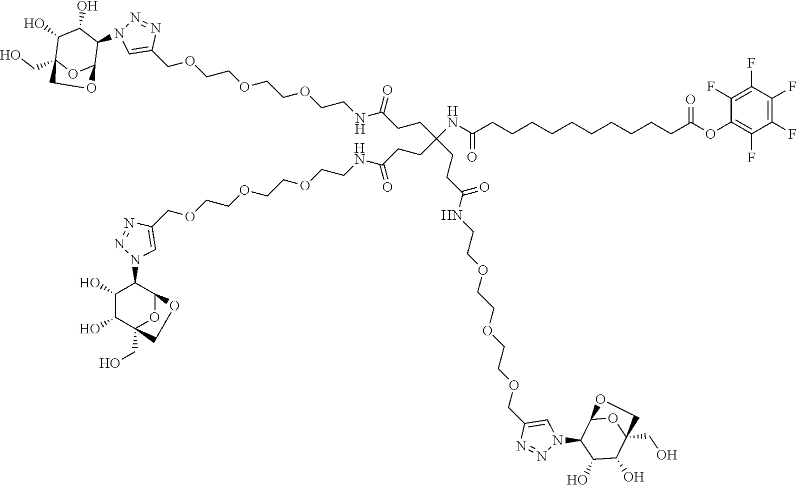 [SMILES: O=C(CCC(CCC(=O)NCCOCCOCCOCc1cn([C@H]2[C@H]3OC[C@](CO)(O3)[C@H](O)[C@@H]2O)nn1)(CCC(=O)NCCOCCOCCOCc1cn([C@H]2[C@H]3OC[C@](CO)(O3)[C@H](O)[C@@H]2O)nn1)NC(=O)CCCCCCCCCCC(=O)Oc1c(F)c(F)c(F)c(F)c1F)NCCOCCOCCOCc1cn([C@H]2[C@H]3OC[C@](CO)(O3)[C@H](O)[C@@H]2O)nn1] |
| 1915 | 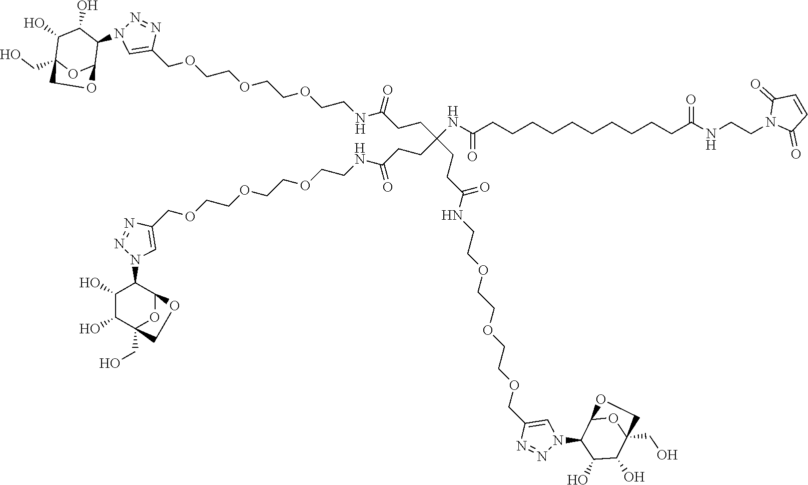 [SMILES: O=C(CCCCCCCCCCC(=O)NC(CCC(=O)NCCOCCOCCOCc1cn([C@H]2[C@H]3OC[C@](CO)(O3)[C@H](O)[C@@H]2O)nn1)(CCC(=O)NCCOCCOCCOCc1cn([C@H]2[C@H]3OC[C@](CO)(O3)[C@H](O)[C@@H]2O)nn1)CCC(=O)NCCOCCOCCOCc1cn([C@H]2[C@H]3OC[C@](CO)(O3)[C@H](O)[C@@H]2O)nn1)NCCN1C(=O)C=CC1=O] |
| 1915 A | 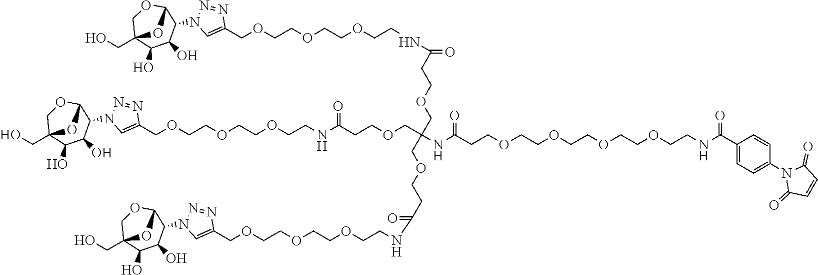 [SMILES: O=C(CCOCC(COCCC(=O)NCCOCCOCCOCc1cn([C@H]2[C@H]3OC[C@](CO)(O3)[C@H](O)[C@@H]2O)nn1)(COCCC(=O)NCCOCCOCCOCc1cn([C@H]2[C@H]3OC[C@](CO)(O3)[C@H](O)[C@@H]2O)nn1)NC(=O)CCOCCOCCOCCOCCNC(=O)c1ccc(N2C(=O)C=CC2=O)cc1)NCCOCCOCCOCc1cn([C@H]2[C@H]3OC[C@](CO)(O3)[C@H](O)[C@@H]2O)nn1] |
| 1916 | 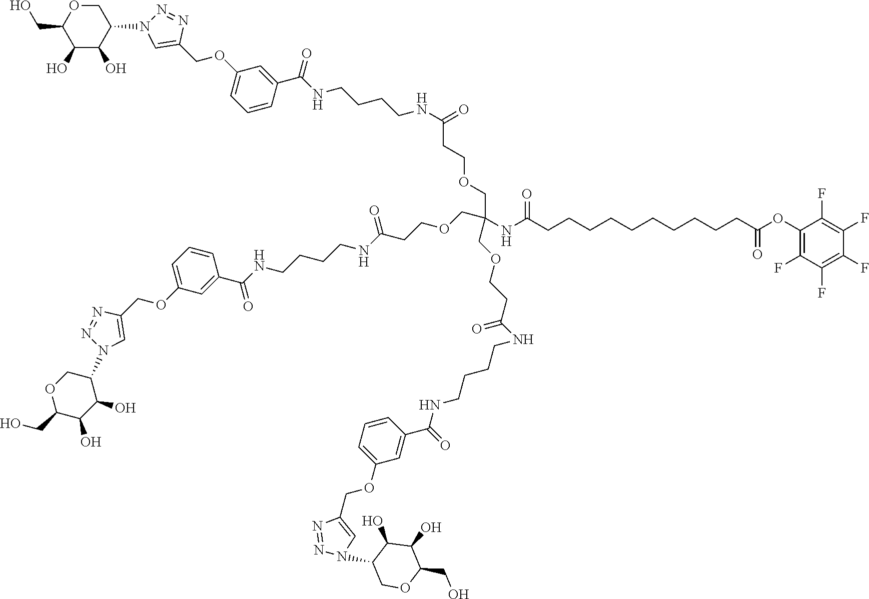 [SMILES: O=C(CCOCC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)NC(=O)CCCCCCCCCCC(=O)Oc1c(F)c(F)c(F)c(F)c1F)NCCCCNC(=O)c1cccc(OCc2cn([C@H]3CO[C@H](CO)[C@H](O)[C@@H]3O)nn2)c1] |
| 1917 | 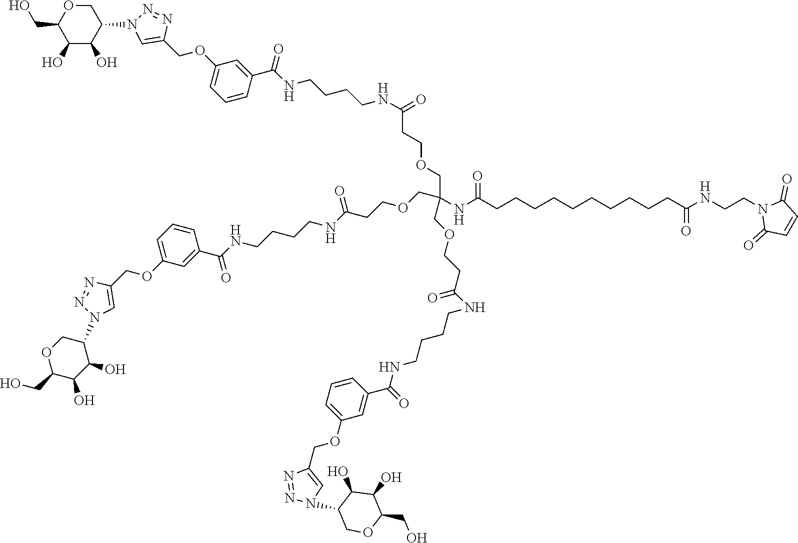 [SMILES: O=C(CCCCCCCCCCC(=O)NC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)COCCC(=O)NCCCCNC(=O)c1cccc(OCc2cn([C@H]3CO[C@H](CO)[C@H](O)[C@@H]3O)nn2)c1)NCCN1C(=O)C=CC1=O] |
| 1918 (I- 165) | 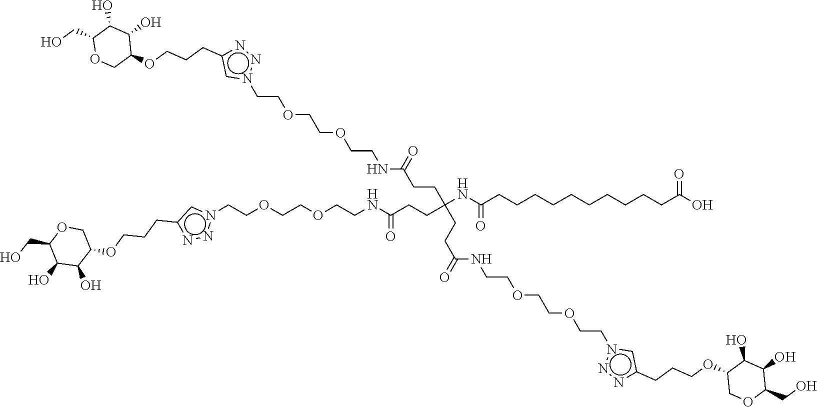 |
| 1919 (I- 166) | 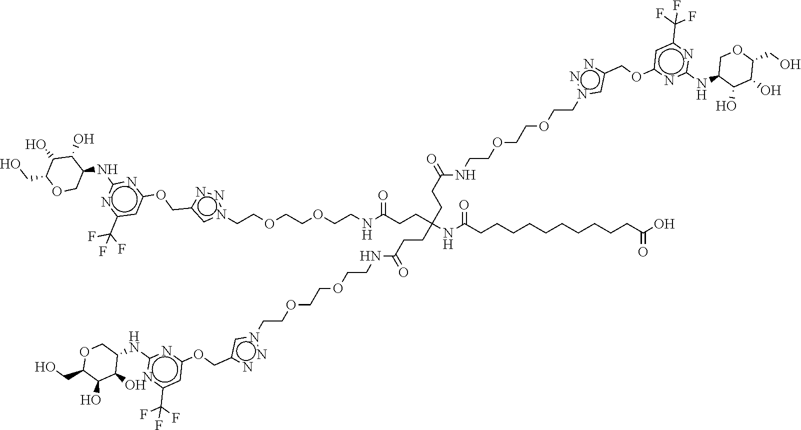 |
| 1920 (I- 167) | 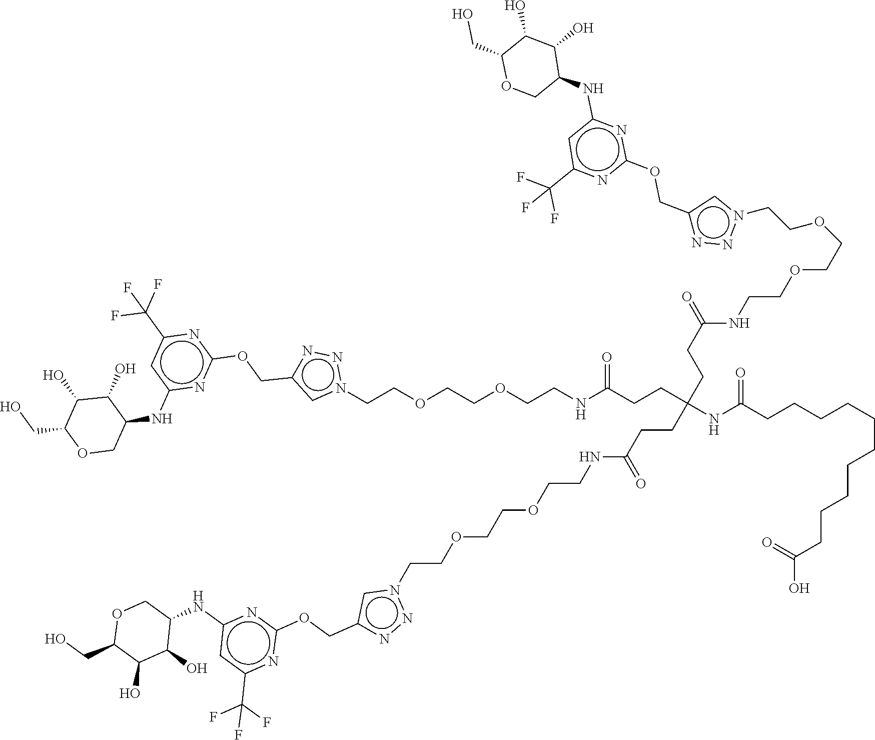 |
| 1921 | 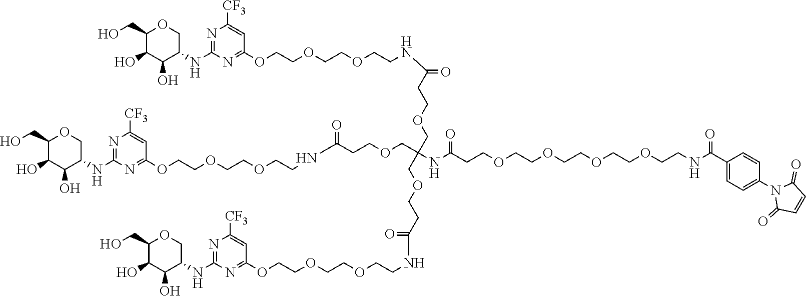 [SMILES: O=C(CCOCC(COCCC(=O)NCCOCCOCCOc1cc(C(F)(F)F)nc(N[C@H]2CO[C@H](CO)[C@H](O)[C@@H]2O)n1)(COCCC(=O)NCCOCCOCCOc1cc(C(F)(F)F)nc(N[C@H]2CO[C@H](CO)[C@H](O)[C@@H]2O)n1)NC(=O)CCOCCOCCOCCOCCNC(=O)c1ccc(N2C(=O)C=CC2=O)cc1)NCCOCCOCCOc1cc(C(F)(F)F)nc(N[C@H]2CO[C@H](CO)[C@H](O)[C@@H]2O)n1] |
| 1922 | 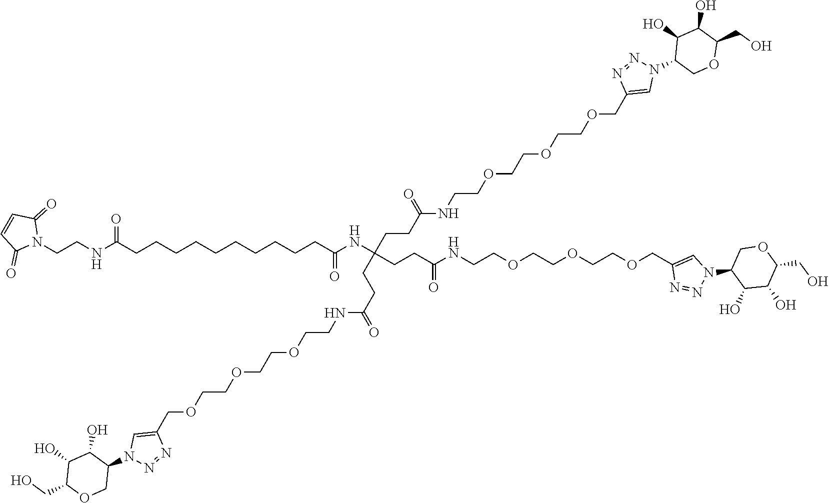 [SMILES: O=C(CCCCCCCCCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1)NCCN1C(=O)C=CC1=O] |
| 1923 | 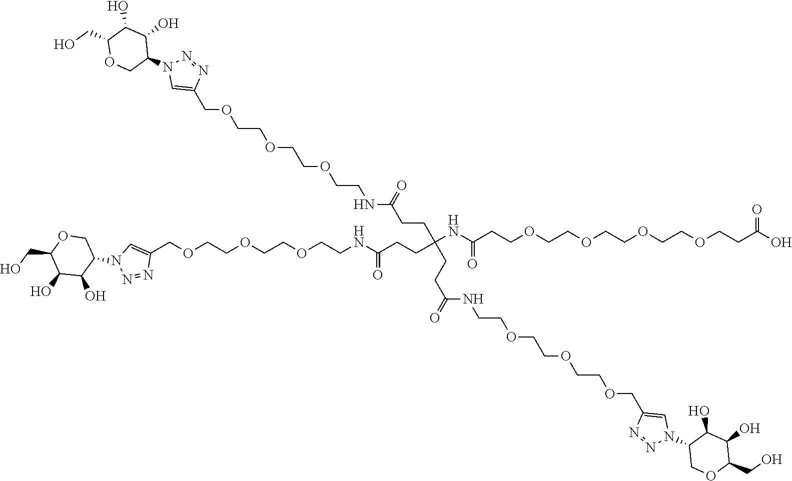 [SMILES: O=C(O)CCOCCOCCOCCOCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1] |
| 1924 | 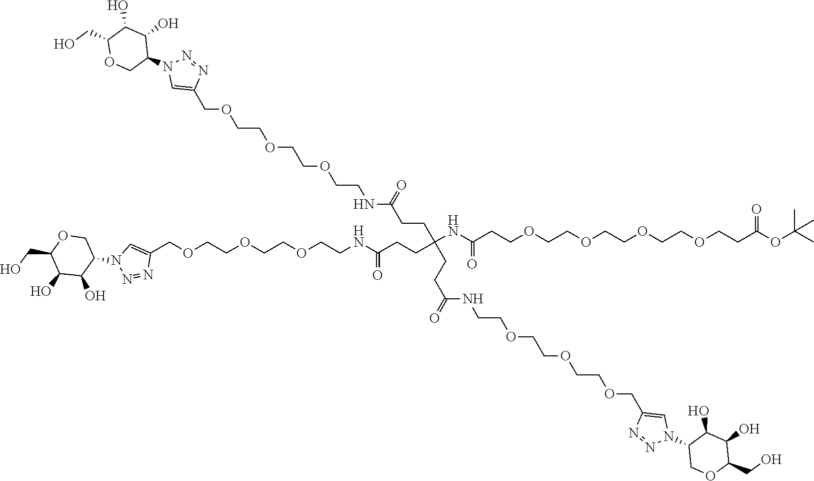 [SMILES: CC(C)(C)OC(=O)CCOCCOCCOCCOCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1] |
| 1925 | 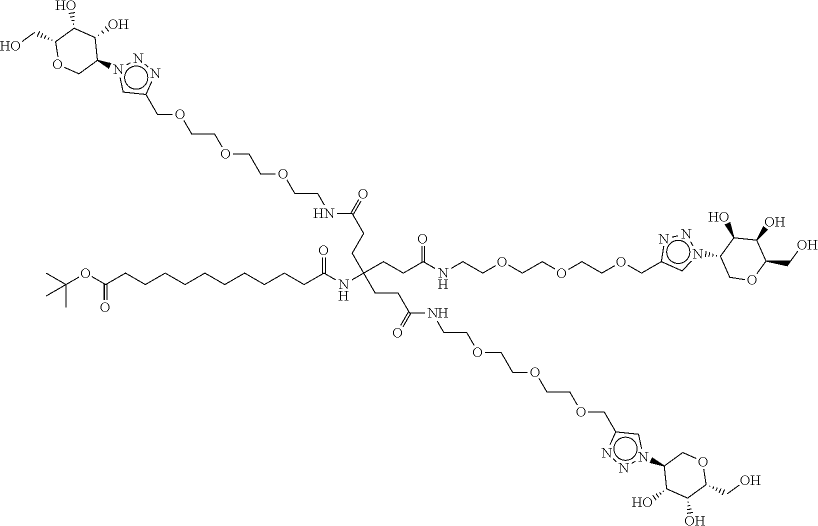 |
| 1926 | 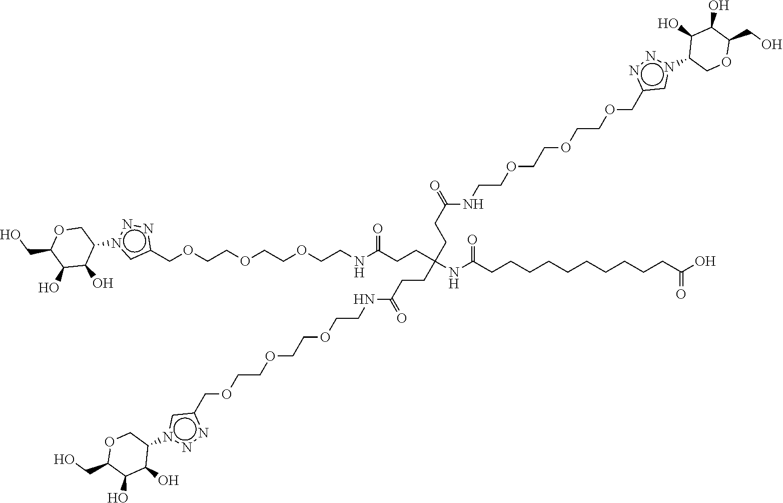 |

Table 20 illustrates exemplary monovalent ASGPR binding intermediate compounds of this disclosure that include a promoiety and X groups that are of formula (Ib).

TABLE 20
Multivalent ASGPR binding intermediate compounds of formula (Ib)

| # | Structure |
|---|---|
| 2001 (I- 173) | 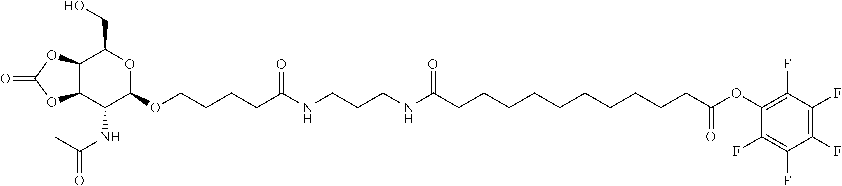 [SMILES: CC(=O)N[C@H]1[C@H](OCCCCC(=O)NCCCNC(=O)CCCCCCCCCCC(=O)Oc2c(F)c(F)c(F)c(F)c2F)O[C@H](CO)[C@@H]2OC(=O)O[C@H]12] |

Table 21 illustrates exemplary ASGPR binding intermediate compounds of this disclosure that include X groups that are of formula (In).

TABLE 21
ASGPR binding intermediate compounds including X groups of formula (In)

| # | Structure |
|---|---|
| 2101 | 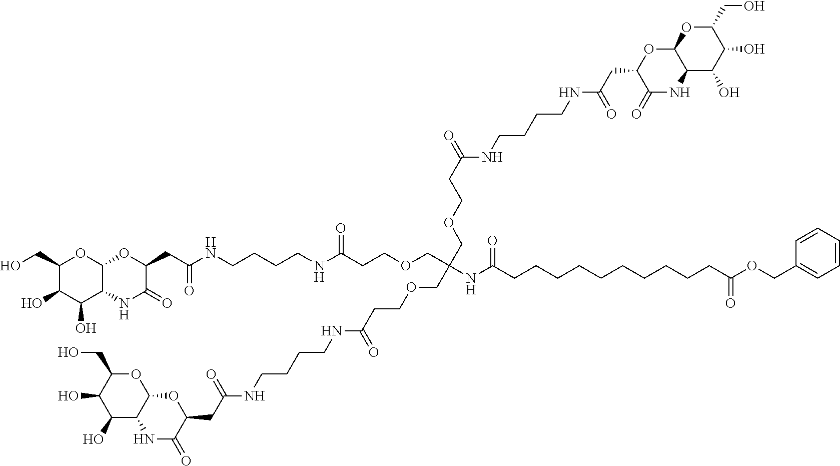 [SMILES: O=C(CCOCC(COCCC(=O)NCCCCNC(=O)C[C@@H]1O[C@H]2O[C@H](CO)[C@H](O)[C@H](O)[C@H]2NC1=O)(COCCC(=O)NCCCCNC(=O)C[C@@H]1O[C@H]2O[C@H](CO)[C@H](O)[C@H](O)[C@H]2NC1=O)NC(=O)CCCCCCCCCCC(=O)OCc1ccccc1)NCCCCNC(=O)C[C@@H]1O[C@H]2O[C@H](CO)[C@H](O)[C@H](O)[C@H]2NC1=O] |

Table 22 illustrates exemplary ASGPR binding intermediate compounds.

TABLE 22
ASGPR binding intermediate compounds

| # | Structure |
|---|---|
| 2301 | 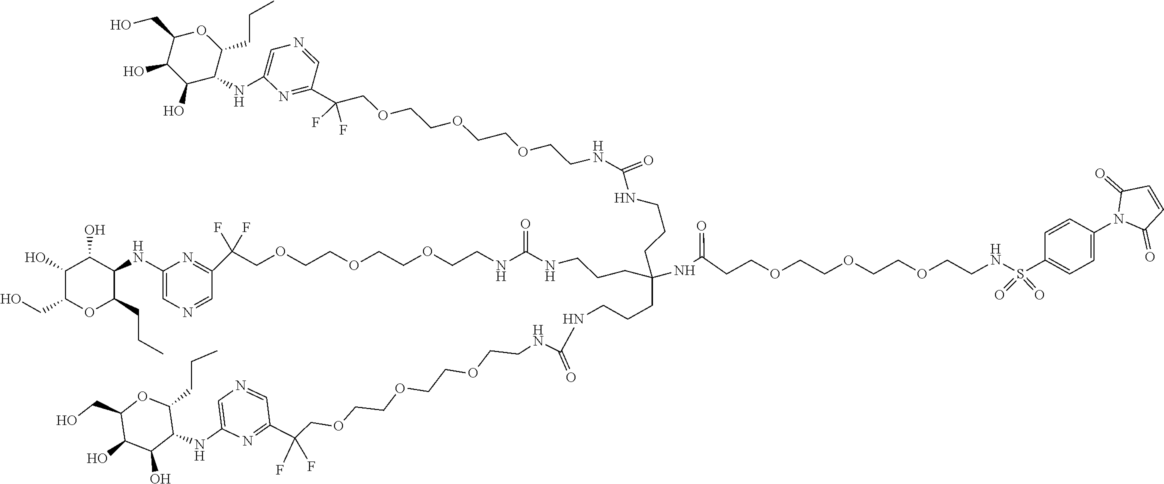 [SMILES: CCC[C@H]1O[C@H](CO)[C@H](O)[C@H](O)[C@H]1Nc1cncc(C(F)(F)COCCOCCOCCNC(=O)NCCCC(CCCNC(=O)NCCOCCOCCOCC(F)(F)c2cncc(N[C@@H]3[C@@H](O)[C@@H](O)[C@@H](CO)O[C@@H]3CCC)n2)(CCCNC(=O)NCCOCCOCCOCC(F)(F)c2cncc(N[C@@H]3[C@@H](O)[C@@H](O)[C@@H](CO)O[C@@H]3CCC)n2)NC(=O)CCOCCOCCOCCNS(=O)(=O)c2ccc(N3C(=O)C=CC3=O)cc2)n1] |
| 2302 | 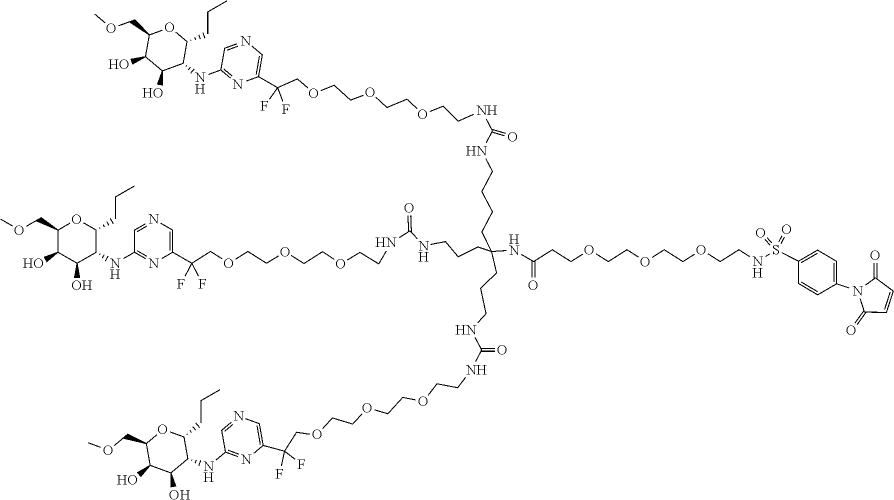 [SMILES: CCC[C@H]1O[C@H](COC)[C@H](O)[C@H](O)[C@H]1Nc1cncc(C(F)(F)COCCOCCOCCNC(=O)NCCCCC(CCCNC(=O)NCCOCCOCCOCC(F)(F)c2cncc(N[C@@H]3[C@@H](O)[C@@H](O)[C@@H](COC)O[C@@H]3CCC)n2)(CCCNC(=O)NCCOCCOCCOCC(F)(F)c2cncc(N[C@@H]3[C@@H](O)[C@@H](O)[C@@H](COC)O[C@@H]3CCC)n2)NC(=O)CCOCCOCCOCCNS(=O)(=O)c2ccc(N3C(=O)C=CC3=O)cc2)n1] |
| 2303 | 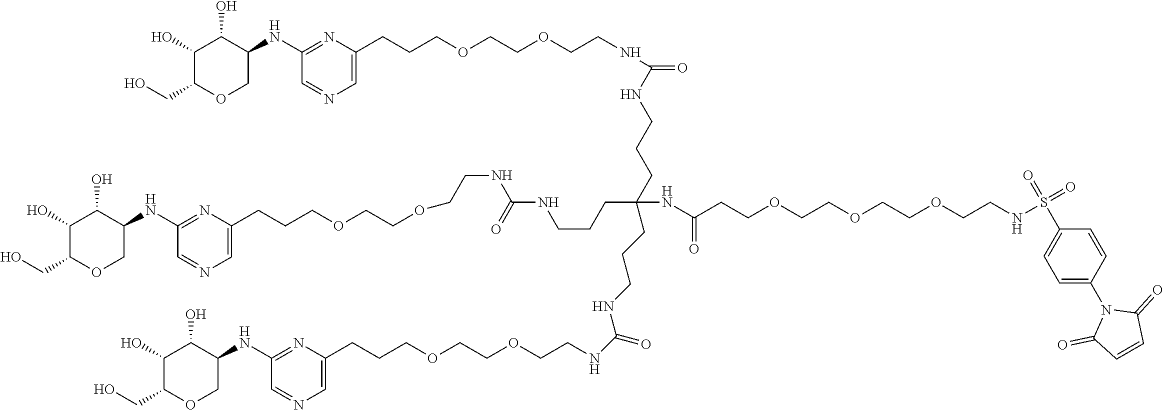 [SMILES: O=C(CCOCCOCCOCCNS(=O)(=O)c1ccc(N2C(=O)C=CC2=O)cc1)NC(CCCNC(=O)NCCOCCOCCCc1cncc(N[C@H]2CO[C@H](CO)[C@H](O)[C@@H]2O)n1)(CCCNC(=O)NCCOCCOCCCc1cncc(N[C@H]2CO[C@H](CO)[C@H](O)[C@@H]2O)n1)CCCNC(=O)NCCOCCOCCCc1cncc(N[C@H]2CO[C@H](CO)[C@H](O)[C@@H]2O)n1] |
| 2304 | 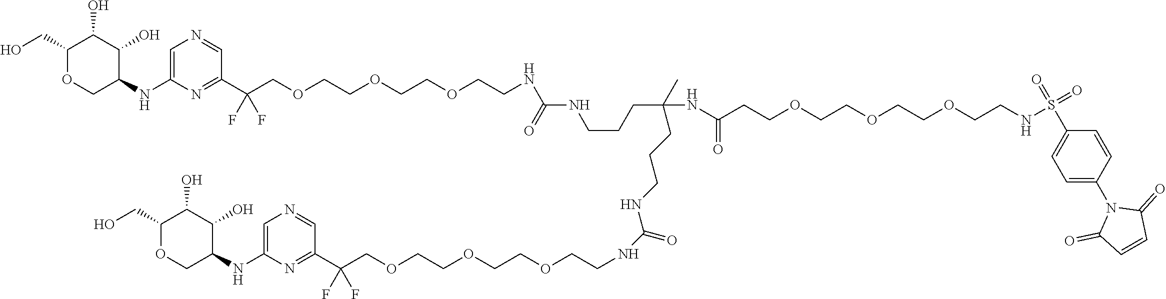 [SMILES: CC(CCCNC(=O)NCCOCCOCCOCC(F)(F)c1cncc(N[C@H]2CO[C@H](CO)[C@H](O)[C@@H]2O)n1)(CCCNC(=O)NCCOCCOCCOCC(F)(F)c1cncc(N[C@H]2CO[C@H](CO)[C@H](O)[C@@H]2O)n1)NC(=O)CCOCCOCCOCCNS(=O)(=O)c1ccc(N2C(=O)C=CC2=O)cc1] |
| 2305 | 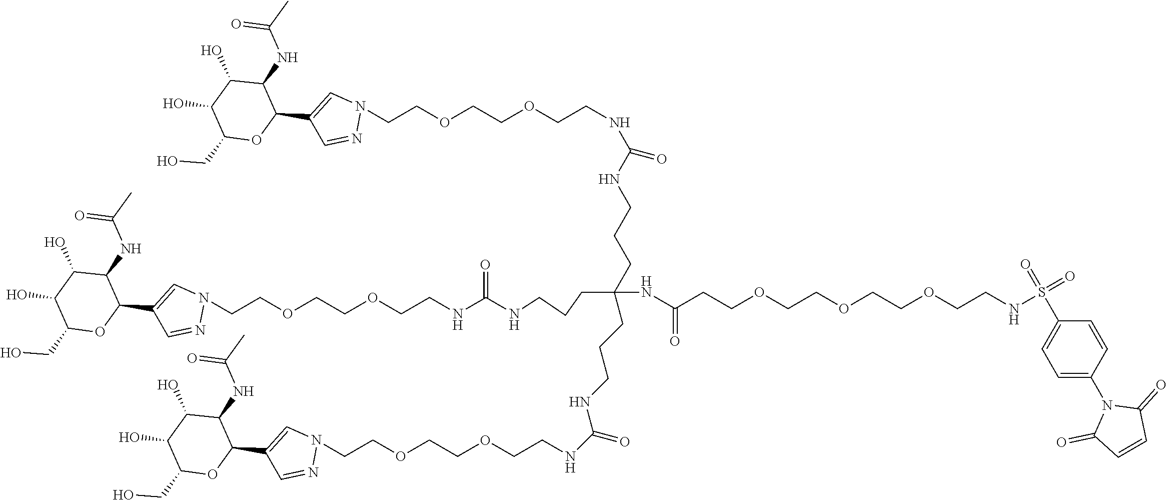 [SMILES: CC(=O)N[C@@H]1[C@@H](O)[C@@H](O)[C@@H](CO)O[C@@H]1c1cnn(CCOCCOCCNC(=O)NCCCC(CCCNC(=O)NCCOCCOCCn2cc([C@H]3O[C@H](CO)[C@H](O)[C@H](O)[C@H]3NC(C)=O)cn2)(CCCNC(=O)NCCOCCOCCn2cc([C@H]3O[C@H](CO)[C@H](O)[C@H](O)[C@H]3NC(C)=O)cn2)NC(=O)CCOCCOCCOCCNS(=O)(=O)c2ccc(N3C(=O)C=CC3=O)cc2)c1] |
| 2306 | 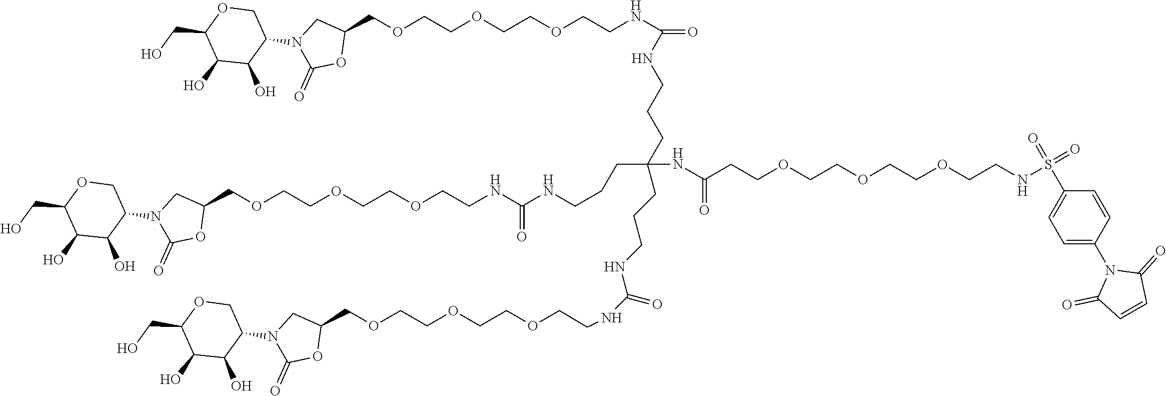 [SMILES: O=C(CCOCCOCCOCCNS(=O)(=O)c1ccc(N2C(=O)C=CC2=O)cc1)NC(CCCNC(=O)NCCOCCOCCOC[C@@H]1CN([C@H]2CO[C@H](CO)[C@H](O)[C@@H]2O)C(=O)O1)(CCCNC(=O)NCCOCCOCCOC[C@@H]1CN([C@H]2CO[C@H](CO)[C@H](O)[C@@H]2O)C(=O)O1)CCCNC(=O)NCCOCCOCCOC[C@@H]1CN([C@H]2CO[C@H](CO)[C@H](O)[C@@H]2O)C(=O)O1] |
| 2307 | 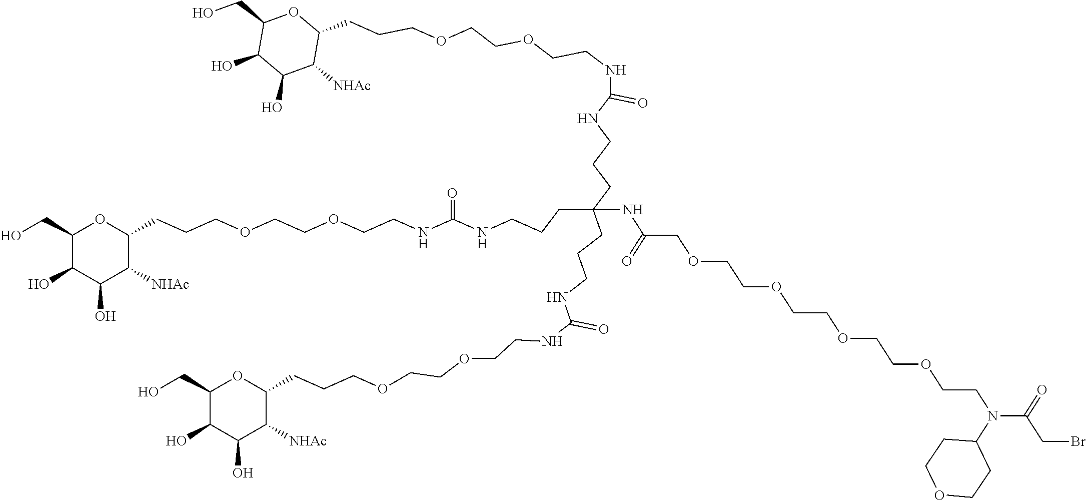 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1] |
| 2308 | 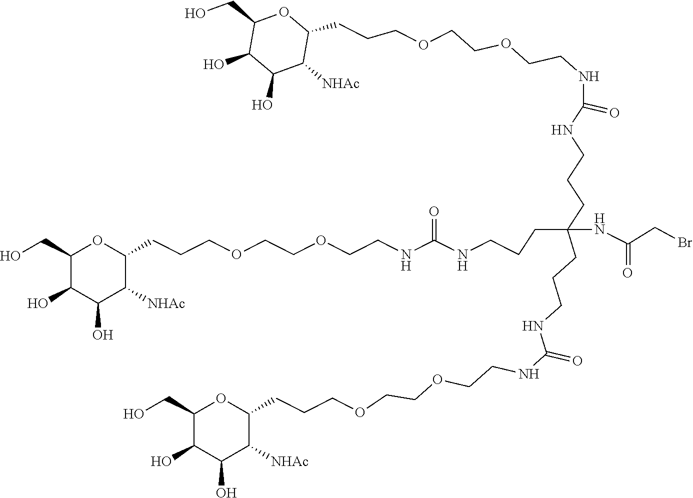 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)CBr] |
| 2309 | 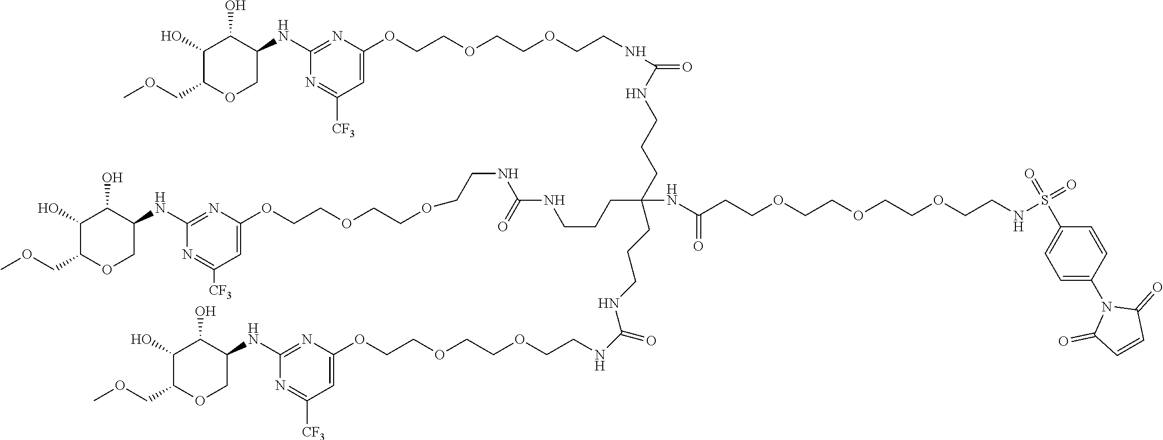 [SMILES: COC[C@H]1OC[C@H](Nc2nc(OCCOCCOCCNC(=O)NCCCC(CCCNC(=O)NCCOCCOCCOc3cc(C(F)(F)F)nc(N[C@H]4CO[C@H](COC)[C@H](O)[C@@H]4O)n3)(CCCNC(=O)NCCOCCOCCOc3cc(C(F)(F)F)nc(N[C@H]4CO[C@H](COC)[C@H](O)[C@@H]4O)n3)NC(=O)CCOCCOCCOCCNS(=O)(=O)c3ccc(N4C(=O)C=CC4=O)cc3)cc(C(F)(F)F)n2)[C@@H](O)[C@H]1O] |
| 2310 | 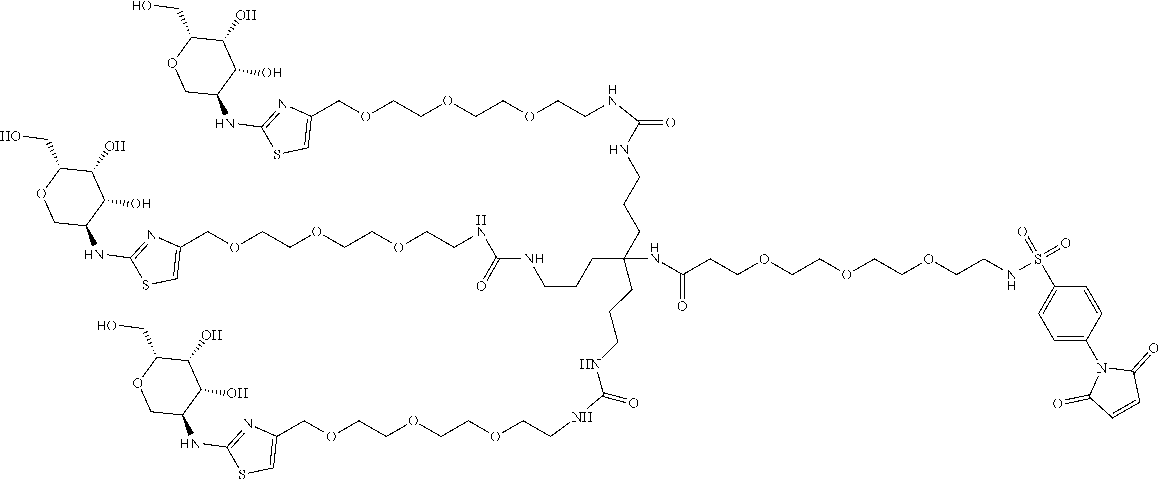 [SMILES: O=C(CCOCCOCCOCCNS(=O)(=O)c1ccc(N2C(=O)C=CC2=O)cc1)NC(CCCNC(=O)NCCOCCOCCOCc1csc(N[C@H]2CO[C@H](CO)[C@H](O)[C@@H]2O)n1)(CCCNC(=O)NCCOCCOCCOCc1csc(N[C@H]2CO[C@H](CO)[C@H](O)[C@@H]2O)n1)CCCNC(=O)NCCOCCOCCOCc1csc(N[C@H]2CO[C@H](CO)[C@H](O)[C@@H]2O)n1] |
| 2311 | 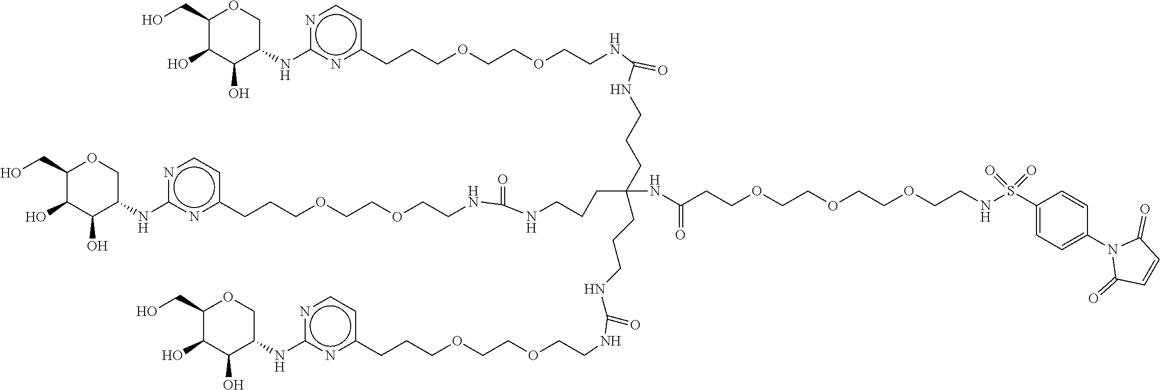 |
| 2312 | 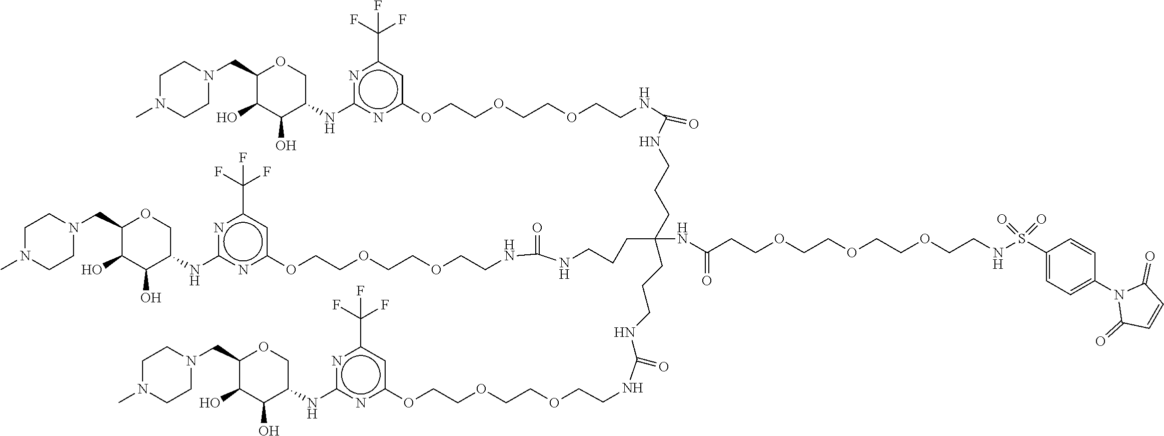 |
| 2313 | 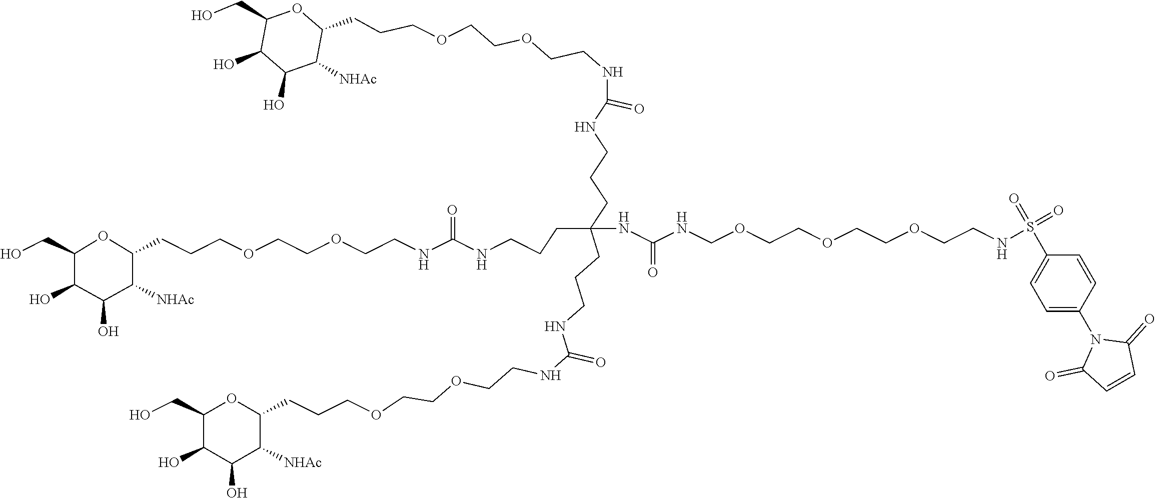 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCOCCOCCOCCNS(=O)(=O)c1ccc(N2C(=O)C=CC2=O)cc1] |
| 2314 | 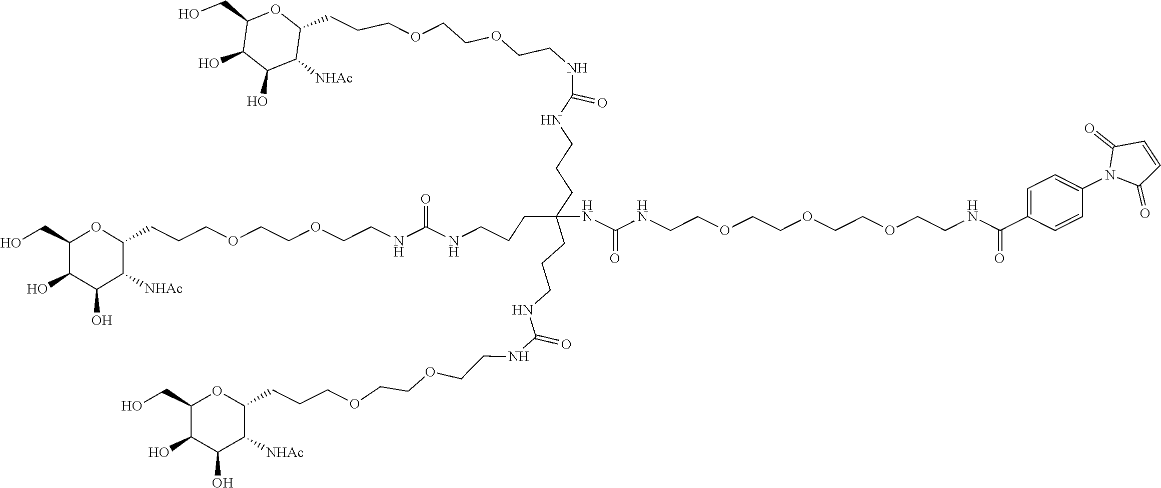 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2315 | 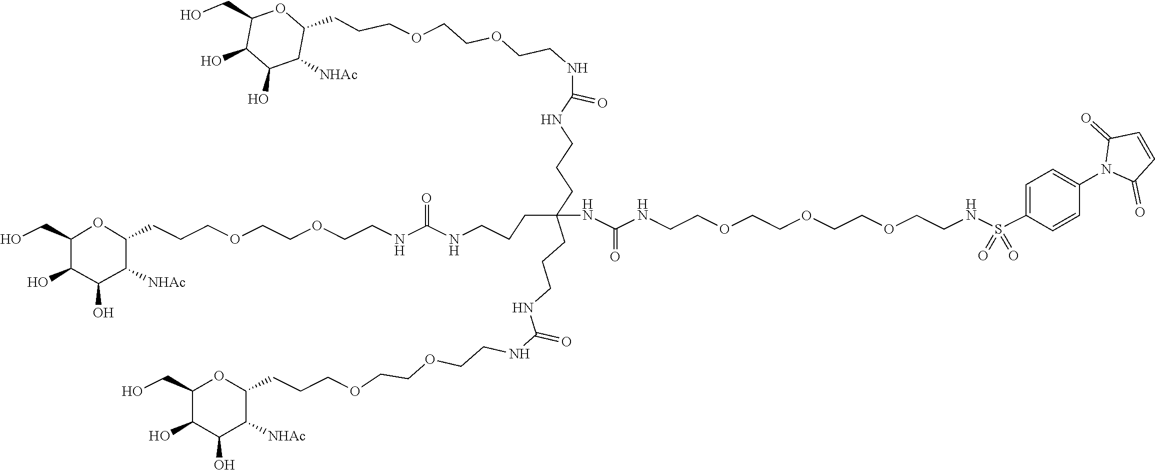 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNS(=O)(=O)c1ccc(N2C(=O)C=CC2=O)cc1] |
| 2316 | 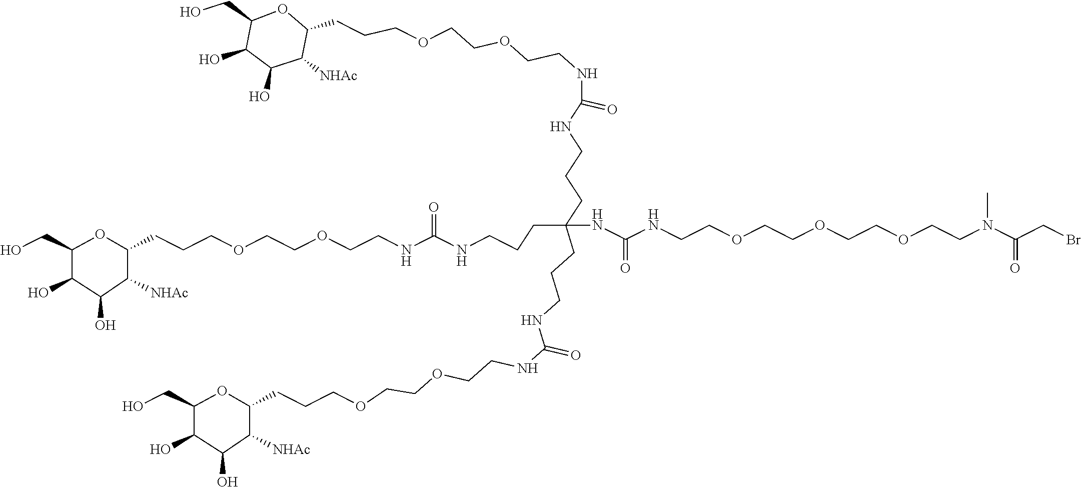 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCN(C)C(=O)CBr] |
| 2317 | 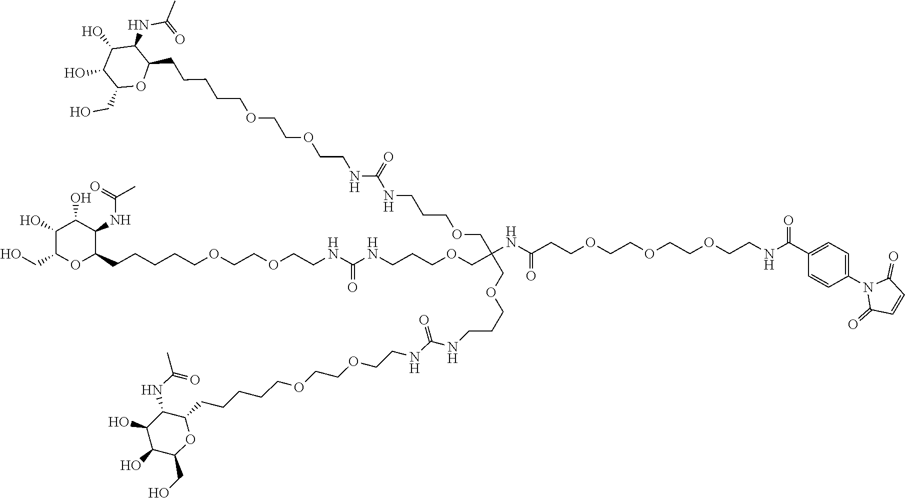 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@@H]1O[C@@H](CO)[C@@H](O)[C@@H](O)[C@@H]1NC(C)=O)(COCCCNC(=O)NCCOCCOCCCCC[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2318 | 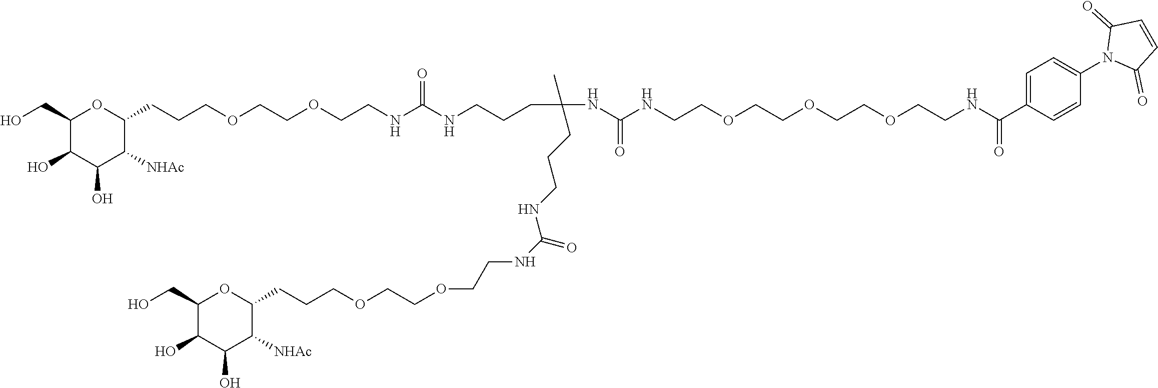 [SMILES: CC(=O)N[C@@H]1[C@@H](O)[C@@H](O)[C@@H](CO)O[C@@H]1CCCOCCOCCNC(=O)NCCCC(C)(CCCNC(=O)NCCOCCOCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2319-C | 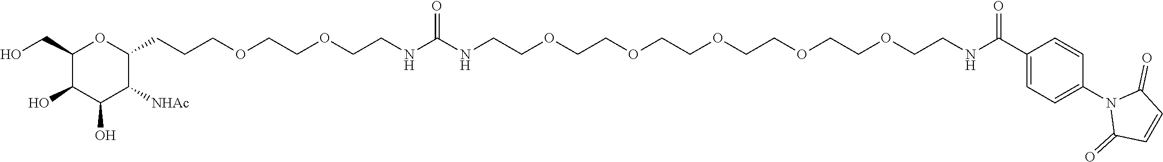 [SMILES: CC(=O)N[C@@H]1[C@@H](O)[C@@H](O)[C@@H](CO)O[C@@H]1CCCOCCOCCNC(=O)NCCOCCOCCOCCOCCOCCNC(=O)c1ccc(N2C(=O)C=CC2=O)cc1] |
| 2320 | 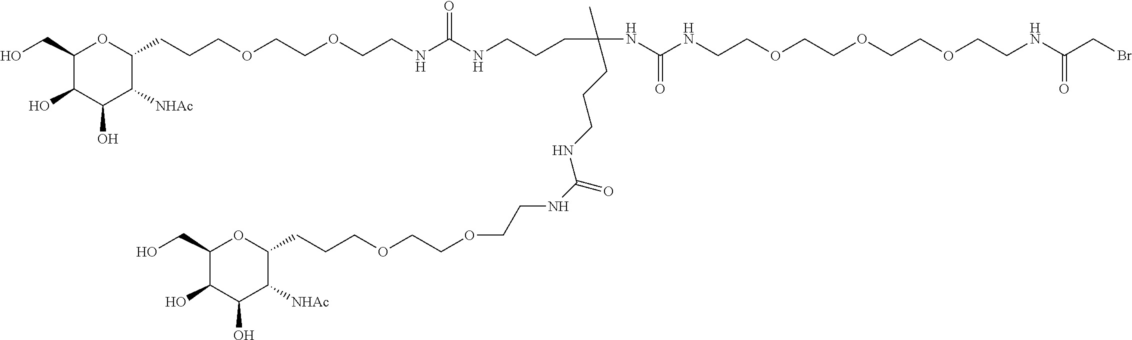 [SMILES: CC(=O)N[C@@H]1[C@@H](O)[C@@H](O)[C@@H](CO)O[C@@H]1CCCOCCOCCNC(=O)NCCCC(C)(CCCNC(=O)NCCOCCOCCC[C@H]1O[C@H](CO)[C@H](O)[C@H](O)[C@H]1NC(C)=O)NC(=O)NCCOCCOCCOCCNC(=O)CBr] |
| 2321 | 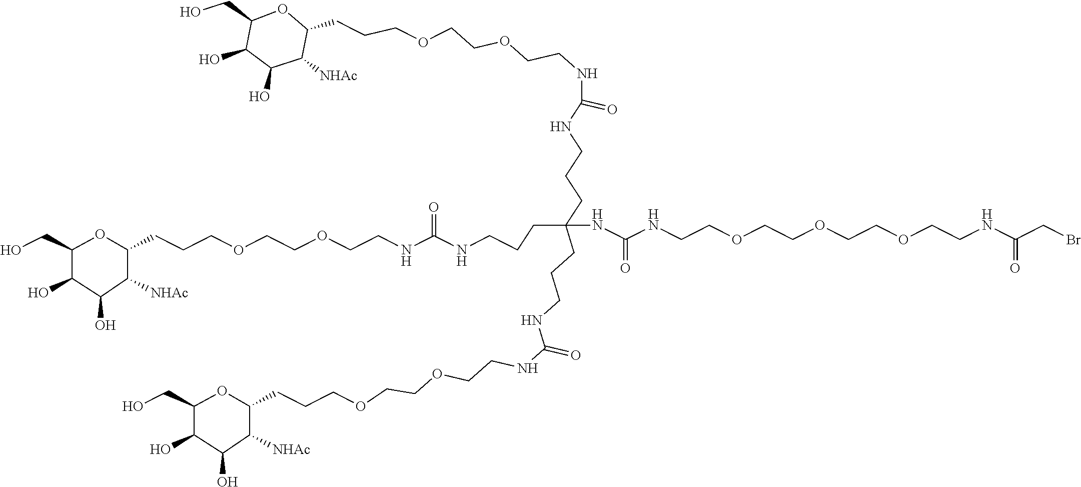 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNC(=O)CBr] |
| 2322 | 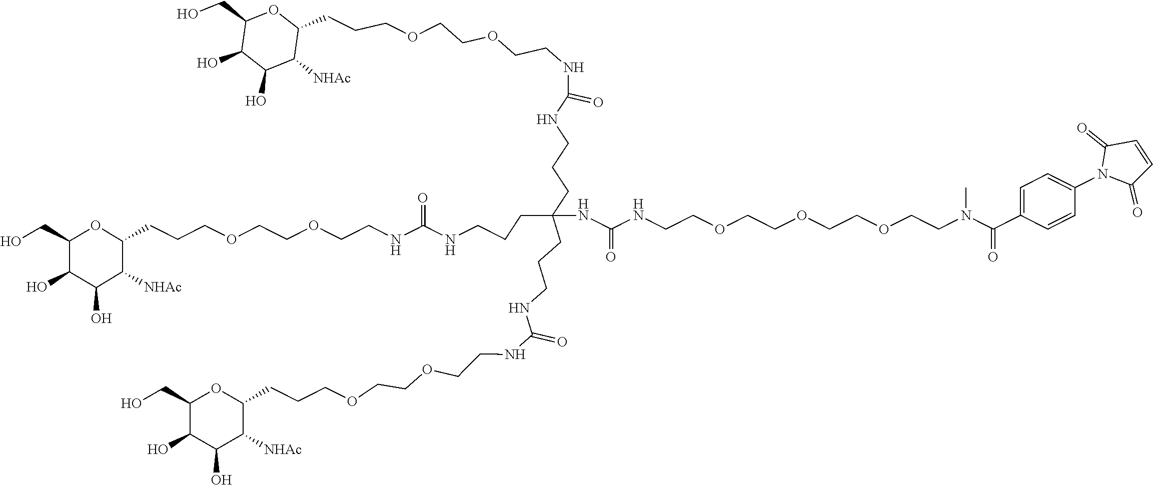 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCN(C)C(=O)c1ccc(N2C(=O)C=CC2=O)cc1] |
| 2323 | 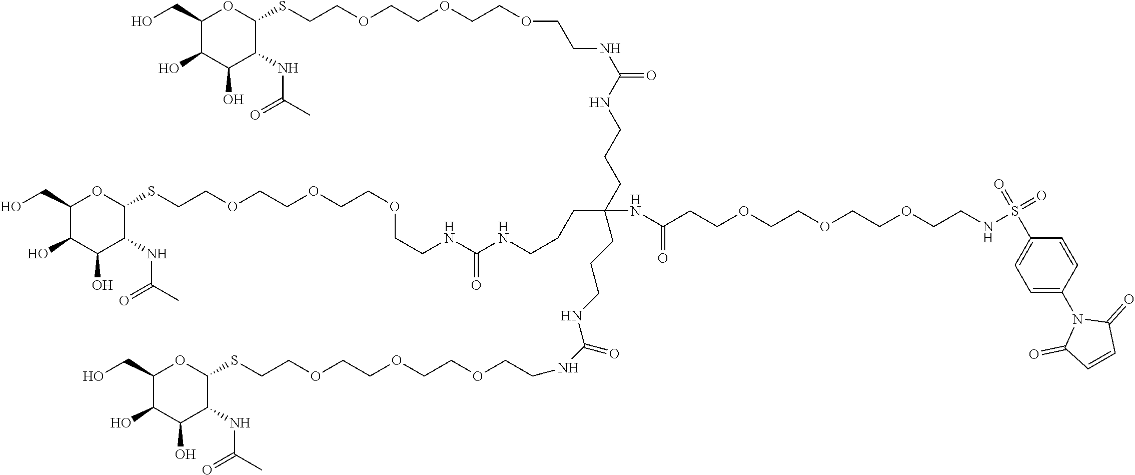 [SMILES: CC(=O)N[C@@H]1[C@@H](O)[C@@H](O)[C@@H](CO)O[C@@H]1SCCOCCOCCOCCNC(=O)NCCCC(CCCNC(=O)NCCOCCOCCOCCS[C@H]1O[C@H](CO)[C@H](O)[C@H](O)[C@H]1NC(C)=O)(CCCNC(=O)NCCOCCOCCOCCS[C@H]1O[C@H](CO)[C@H](O)[C@H](O)[C@H]1NC(C)=O)NC(=O)CCOCCOCCOCCNS(=O)(=O)c1ccc(N2C(=O)C=CC2=O)cc1] |
| 2324 | 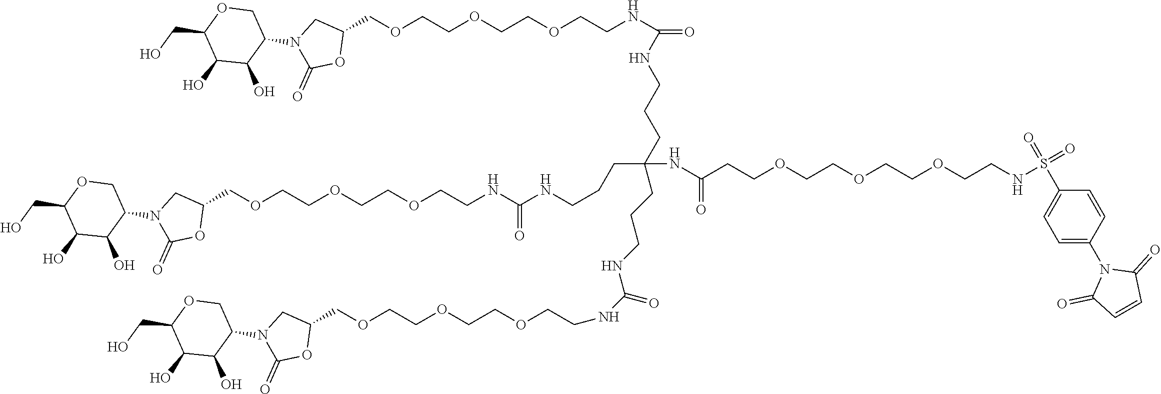 [SMILES: O=C(CCOCCOCCOCCNS(=O)(=O)c1ccc(N2C(=O)C=CC2=O)cc1)NC(CCCNC(=O)NCCOCCOCCOC[C@H]1CN([C@H]2CO[C@H](CO)[C@H](O)[C@@H]2O)C(=O)O1)(CCCNC(=O)NCCOCCOCCOC[C@H]1CN([C@H]2CO[C@H](CO)[C@H](O)[C@@H]2O)C(=O)O1)CCCNC(=O)NCCOCCOCCOC[C@H]1CN([C@H]2CO[C@H](CO)[C@H](O)[C@@H]2O)C(=O)O1] |
| 2325 | 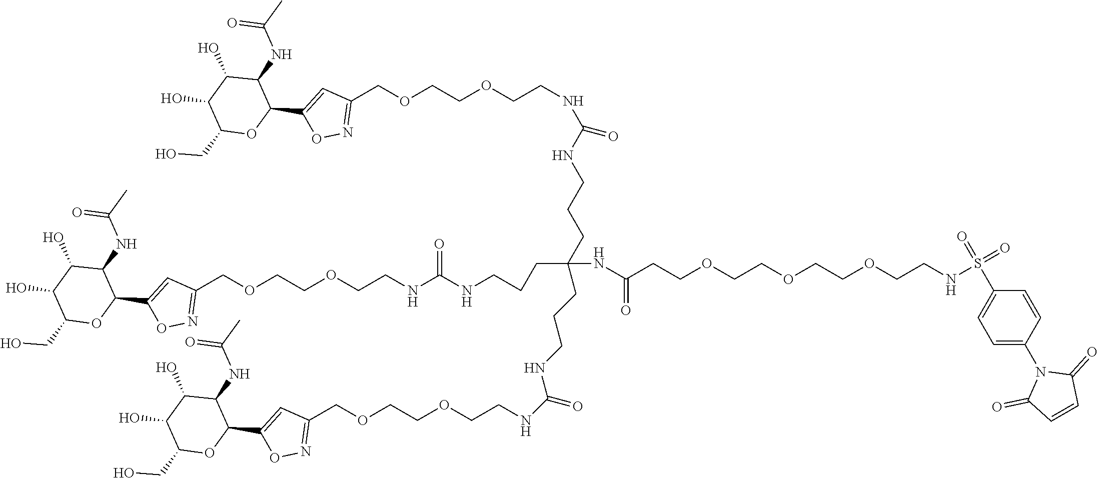 [SMILES: CC(=O)N[C@@H]1[C@@H](O)[C@@H](O)[C@@H](CO)O[C@@H]1c1cc(COCCOCCNC(=O)NCCCC(CCCNC(=O)NCCOCCOCc2cc([C@H]3O[C@H](CO)[C@H](O)[C@H](O)[C@H]3NC(C)=O)on2)(CCCNC(=O)NCCOCCOCc2cc([C@H]3O[C@H](CO)[C@H](O)[C@H](O)[C@H]3NC(C)=O)on2)NC(=O)CCOCCOCCOCCNS(=O)(=O)c2ccc(N3C(=O)C=CC3=O)cc2)no1] |
| 2326 | 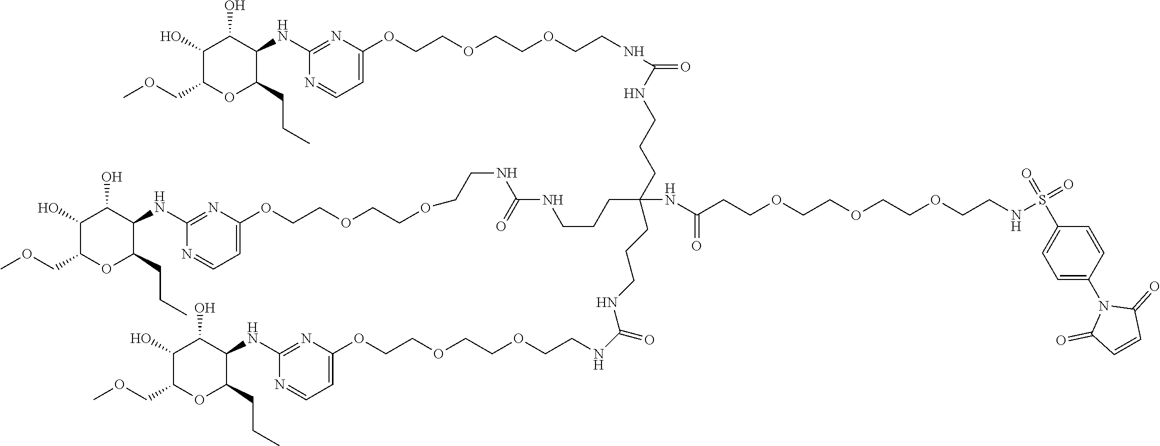 [SMILES: CCC[C@H]1O[C@H](COC)[C@H](O)[C@H](O)[C@H]1Nc1nccc(OCCOCCOCCNC(=O)NCCCC(CCCNC(=O)NCCOCCOCCOc2ccnc(N[C@@H]3[C@@H](O)[C@@H](O)[C@@H](COC)O[C@@H]3CCC)n2)(CCCNC(=O)NCCOCCOCCOc2ccnc(N[C@@H]3[C@@H](O)[C@@H](O)[C@@H](COC)O[C@@H]3CCC)n2)NC(=O)CCOCCOCCOCCNS(=O)(=O)c2ccc(N3C(=O)C=CC3=O)cc2)n1] |
| 2327 | 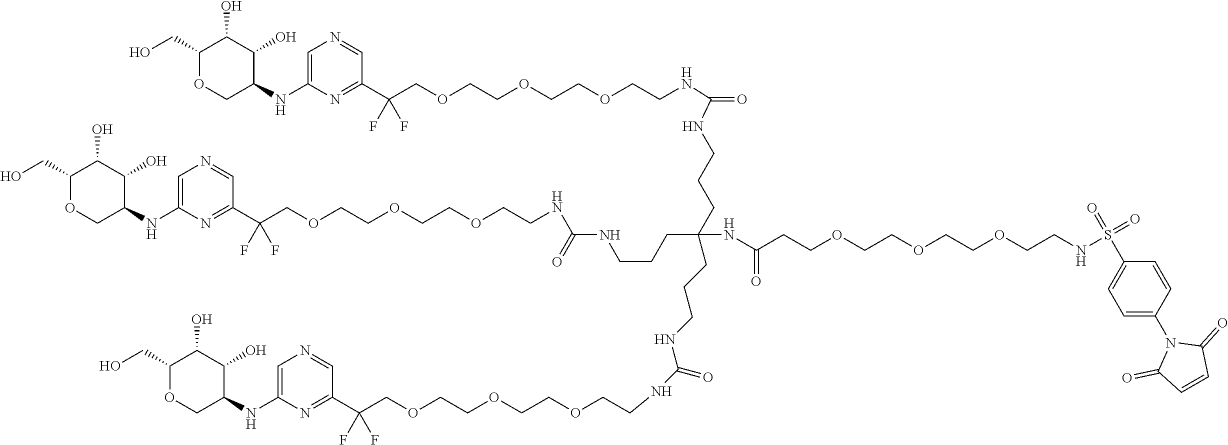 [SMILES: O=C(CCOCCOCCOCCNS(=O)(=O)c1ccc(N2C(=O)C=CC2=O)cc1)NC(CCCNC(=O)NCCOCCOCCOCC(F)(F)c1cncc(N[C@H]2CO[C@H](CO)[C@H](O)[C@@H]2O)n1)(CCCNC(=O)NCCOCCOCCOCC(F)(F)c1cncc(N[C@H]2CO[C@H](CO)[C@H](O)[C@@H]2O)n1)CCCNC(=O)NCCOCCOCCOCC(F)(F)c1cncc(N[C@H]2CO[C@H](CO)[C@H](O)[C@@H]2O)n1] |
| 2328 | 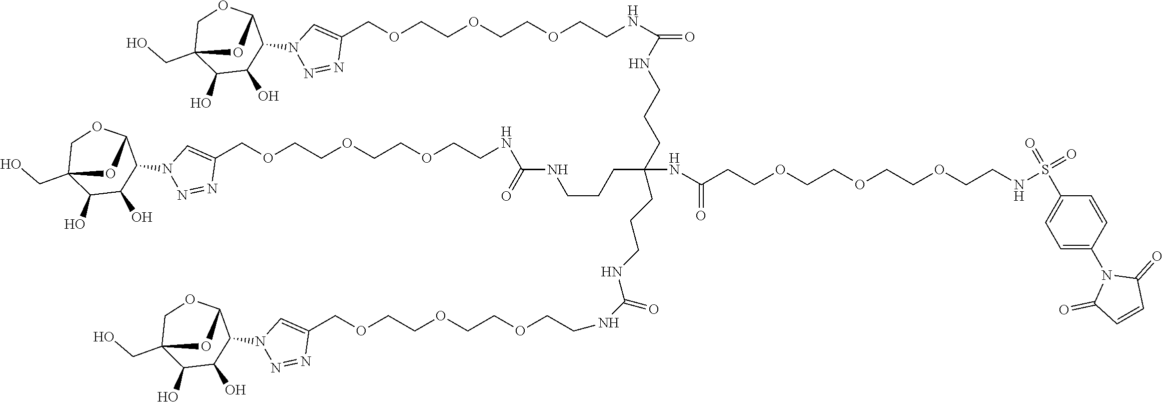 [SMILES: O=C(CCOCCOCCOCCNS(=O)(=O)c1ccc(N2C(=O)C=CC2=O)cc1)NC(CCCNC(=O)NCCOCCOCCOCc1cn([C@H]2[C@H]3OC[C@](CO)(O3)[C@H](O)[C@@H]2O)nn1)(CCCNC(=O)NCCOCCOCCOCc1cn([C@H]2[C@H]3OC[C@](CO)(O3)[C@H](O)[C@@H]2O)nn1)CCCNC(=O)NCCOCCOCCOCc1cn([C@H]2[C@H]3OC[C@](CO)(O3)[C@H](O)[C@@H]2O)nn1] |
| 2329 | 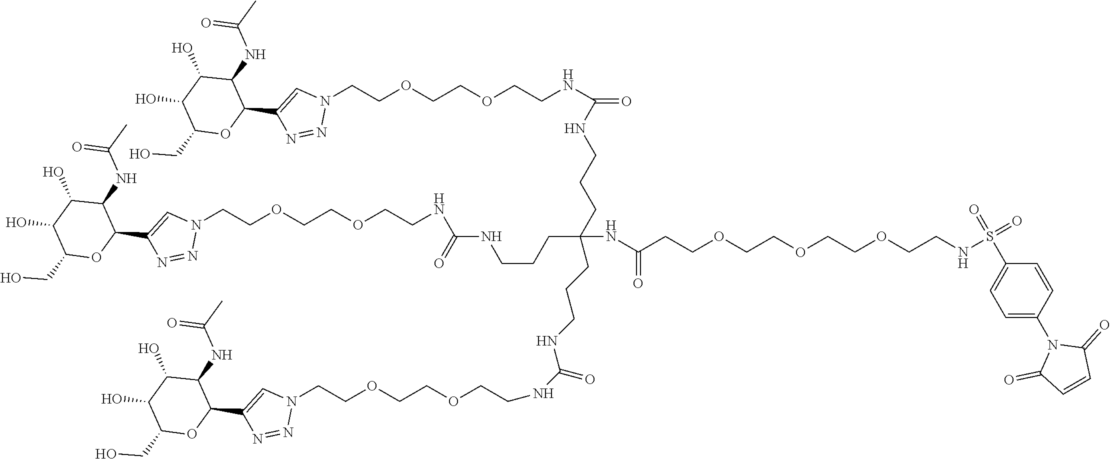 [SMILES: CC(=O)N[C@@H]1[C@@H](O)[C@@H](O)[C@@H](CO)O[C@@H]1c1cn(CCOCCOCCNC(=O)NCCCC(CCCNC(=O)NCCOCCOCCn2cc([C@H]3O[C@H](CO)[C@H](O)[C@H](O)[C@H]3NC(C)=O)nn2)(CCCNC(=O)NCCOCCOCCn2cc([C@H]3O[C@H](CO)[C@H](O)[C@H](O)[C@H]3NC(C)=O)nn2)NC(=O)CCOCCOCCOCCNS(=O)(=O)c2ccc(N3C(=O)C=CC3=O)cc2)nn1] |
| 2330 | 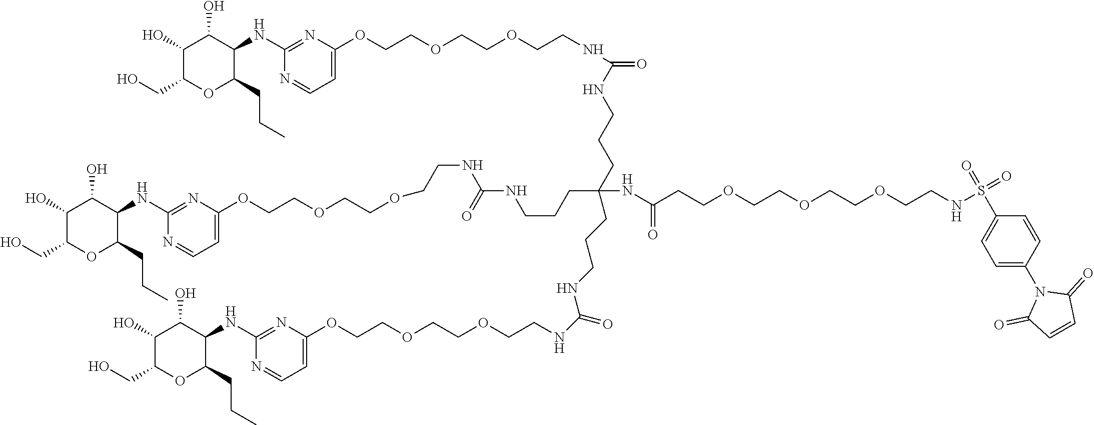 [SMILES: CCC[C@H]1O[C@H](CO)[C@H](O)[C@H](O)[C@H]1Nc1nccc(OCCOCCOCCNC(=O)NCCCC(CCCNC(=O)NCCOCCOCCOc2ccnc(N[C@@H]3[C@@H](O)[C@@H](O)[C@@H](CO)O[C@@H]3CCC)n2)(CCCNC(=O)NCCOCCOCCOc2ccnc(N[C@@H]3[C@@H](O)[C@@H](O)[C@@H](CO)O[C@@H]3CCC)n2)NC(=O)CCOCCOCCOCCNS(=O)(=O)c2ccc(N3C(=O)C=CC3=O)cc2)n1] |
| 2331 | 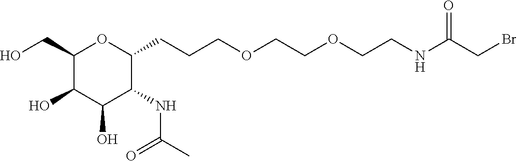 [SMILES: CC(=O)N[C@@H]1[C@@H](O)[C@@H](O)[C@@H](CO)O[C@@H]1CCCOCCOCCNC(=O)CBr] |
| 2332 | 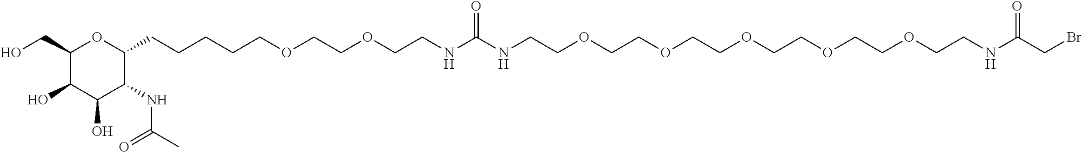 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)CBr] |
| 2333 | 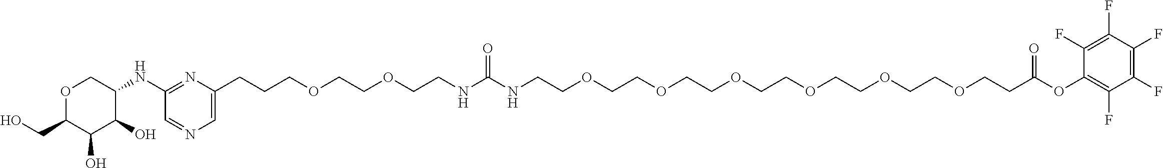 [SMILES: O=C(NCCOCCOCCCc1cncc(N[C@H]2CO[C@H](CO)[C@H](O)[C@@H]2O)n1)NCCOCCOCCOCCOCCOCCOCCC(=O)Oc1c(F)c(F)c(F)c(F)c1F] |
| 2334 | 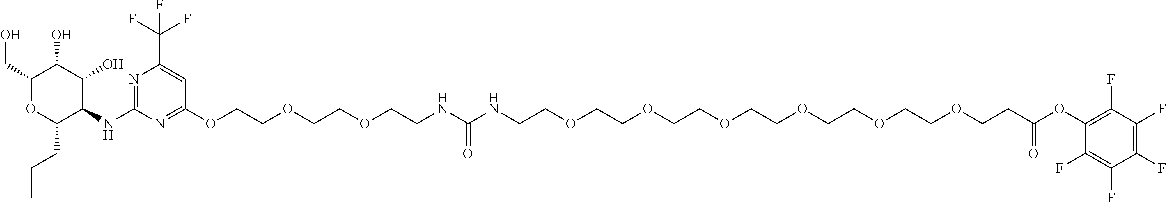 [SMILES: CCC[C@@H]1O[C@H](CO)[C@H](O)[C@H](O)[C@H]1Nc1nc(OCCOCCOCCNC(=O)NCCOCCOCCOCCOCCOCCOCCC(=O)Oc2c(F)c(F)c(F)c(F)c2F)cc(C(F)(F)F)n1] |
| 2335 | 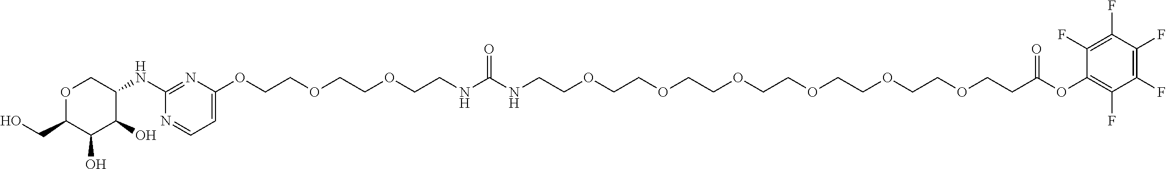 [SMILES: O=C(NCCOCCOCCOCCOCCOCCOCCC(=O)Oc1c(F)c(F)c(F)c(F)c1F)NCCOCCOCCOc1ccnc(N[C@H]2CO[C@H](CO)[C@H](O)[C@@H]2O)n1] |
| 2336 | 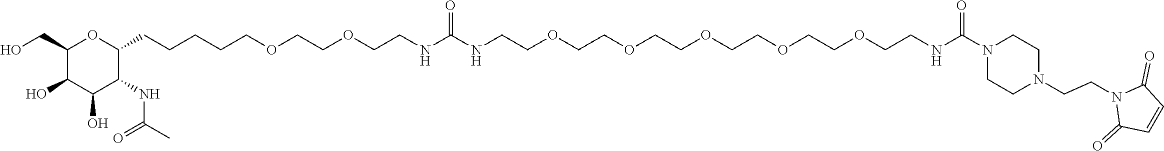 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)N1CCN(CCN2C(=O)C=CC2=O)CC1] |
| 2337 | 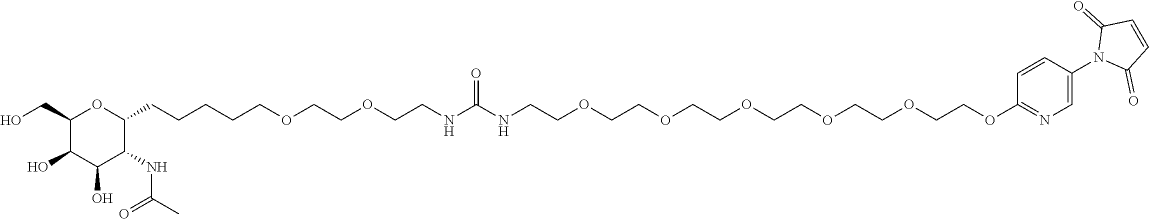 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOc1ccc(N2C(=O)C=CC2=O)cn1] |
| 2338 | 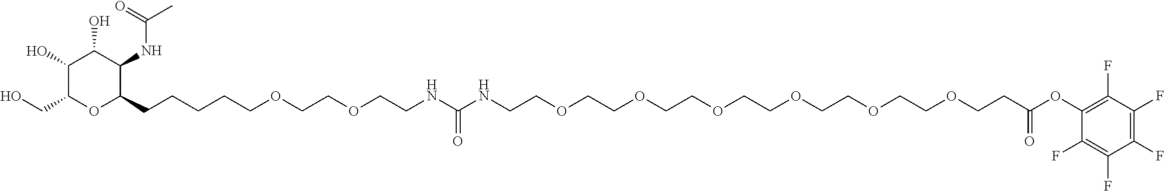 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)Oc1c(F)c(F)c(F)c(F)c1F] |
| 2339 | 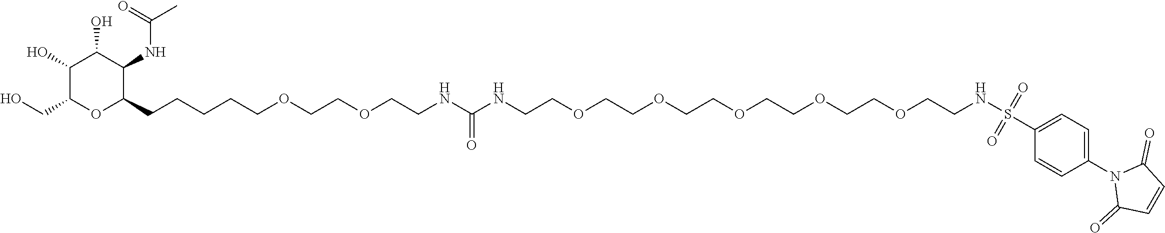 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNS(=O)(=O)c1ccc(N2C(=O)C=CC2=O)cc1] |
| 2340 | 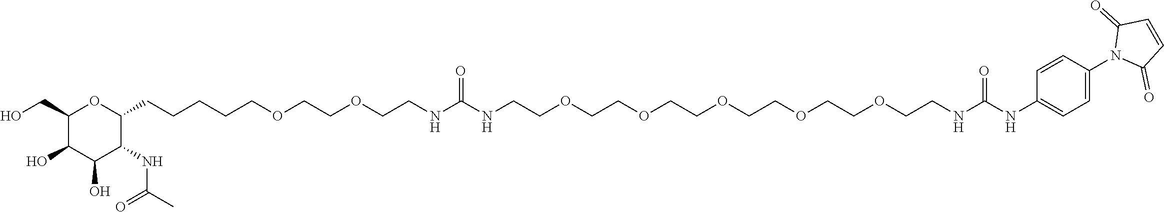 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)Nc1ccc(N2C(=O)C=CC2=O)cc1] |
| 2341 | 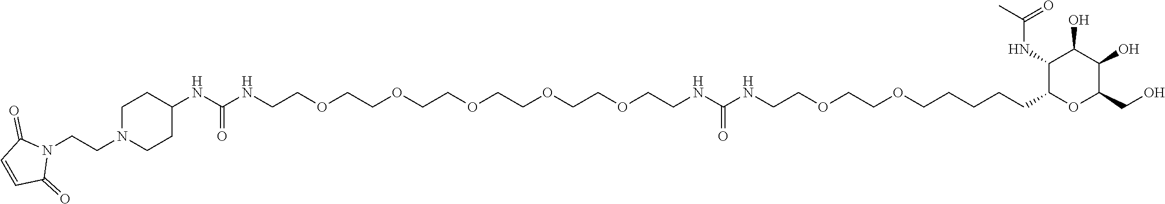 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)NC1CCN(CCN2C(=O)C=CC2=O)CC1] |
| 2342 | 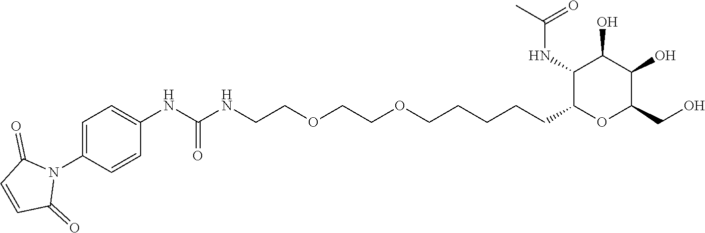 [SMILES: CC(=O)N[C@@H]1[C@@H](O)[C@@H](O)[C@@H](CO)O[C@@H]1CCCCCOCCOCCNC(=O)Nc1ccc(N2C(=O)C=CC2=O)cc1] |
| 2343 | 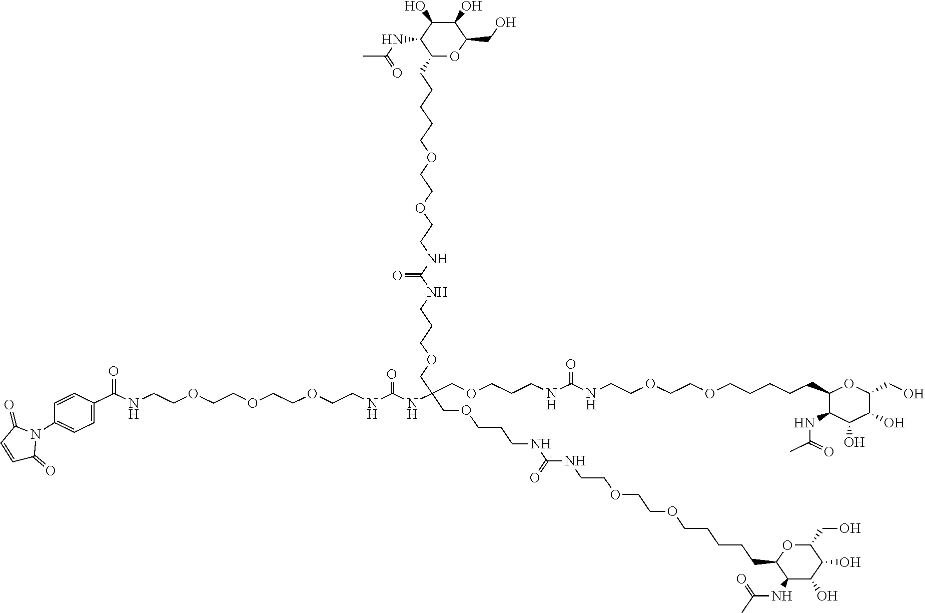 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)(COCCCNC(=O)NCCOCCOCCCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2344 | 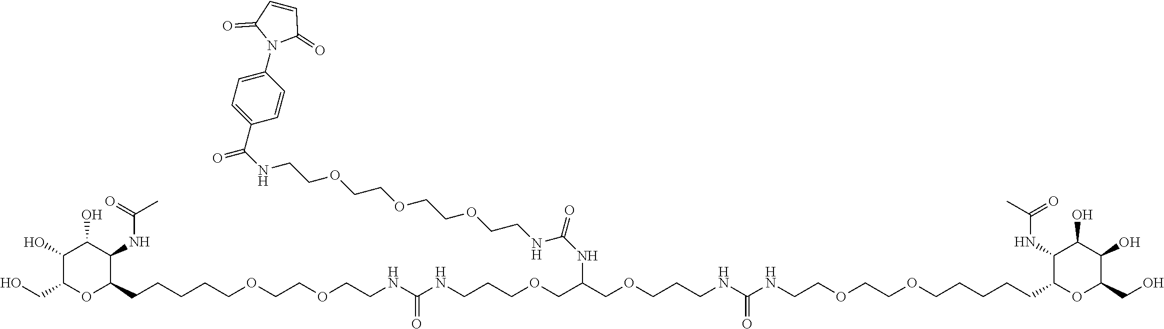 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2345 | 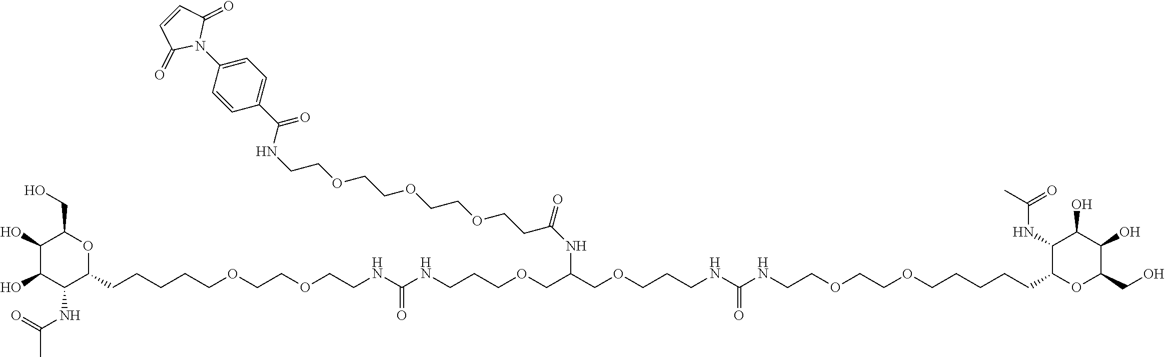 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2346 | 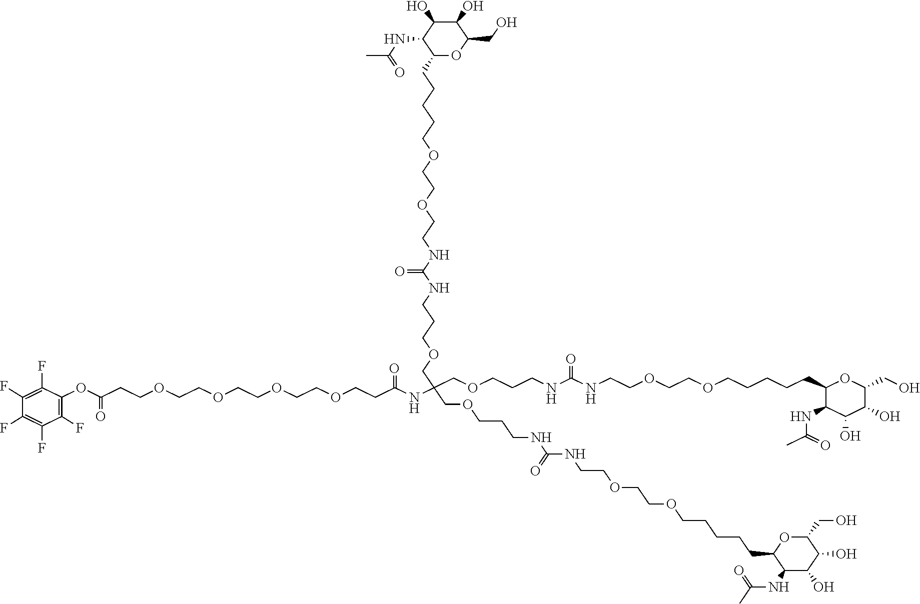 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)(COCCCNC(=O)NCCOCCOCCCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2347 | 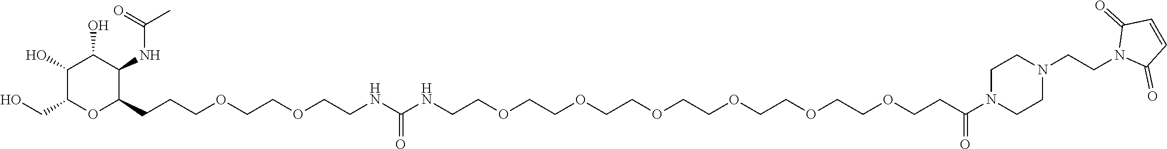 [SMILES: CC(=O)N[C@@H]1[C@@H](O)[C@@H](O)[C@@H](CO)O[C@@H]1CCCOCCOCCNC(=O)NCCOCCOCCOCCOCCOCCOCCC(=O)N1CCN(CCN2C(=O)C=CC2=O)CC1] |
| 2348 | 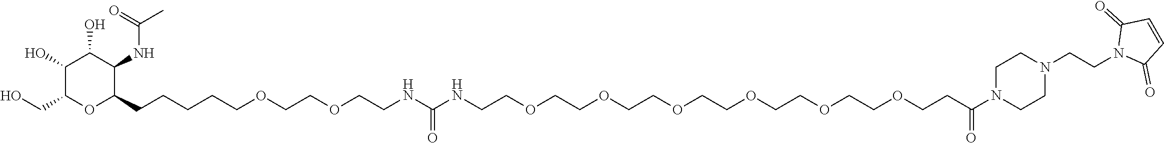 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)N1CCN(CCN2C(=O)C=CC2=O)CC1] |
| 2349 | 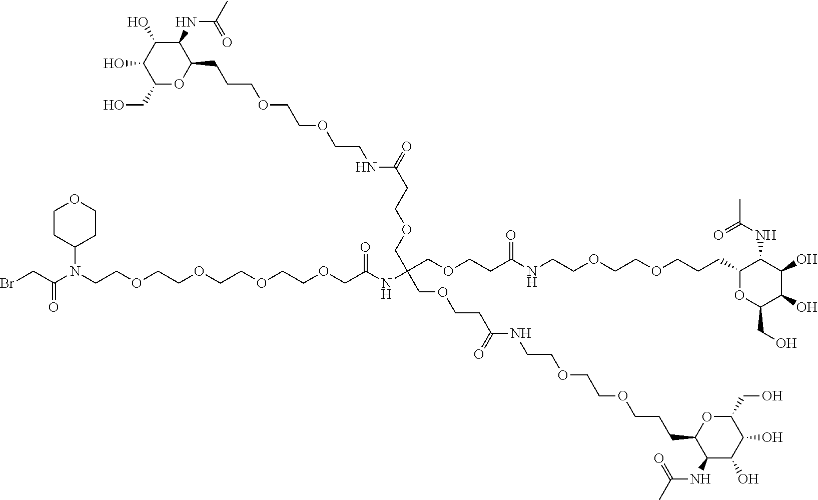 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1] |
| 2350 | 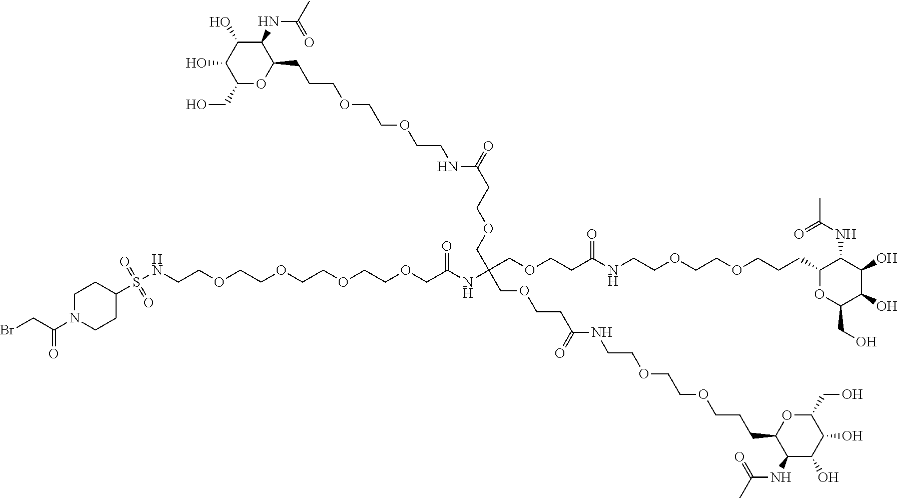 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)COCCOCCOCCOCCNS(=O)(=O)C1CCN(C(=O)CBr)CC1] |
| 2351 | 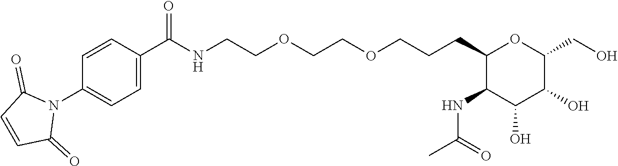 [SMILES: CC(=O)N[C@@H]1[C@@H](O)[C@@H](O)[C@@H](CO)O[C@@H]1CCCOCCOCCNC(=O)c1ccc(N2C(=O)C=CC2=O)cc1] |
| 2352 | 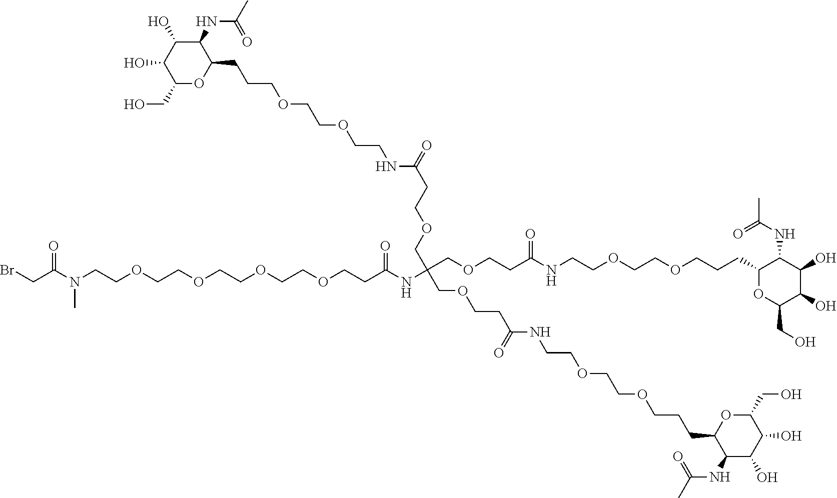 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCN(C)C(=O)CBr] |
| 2353 | 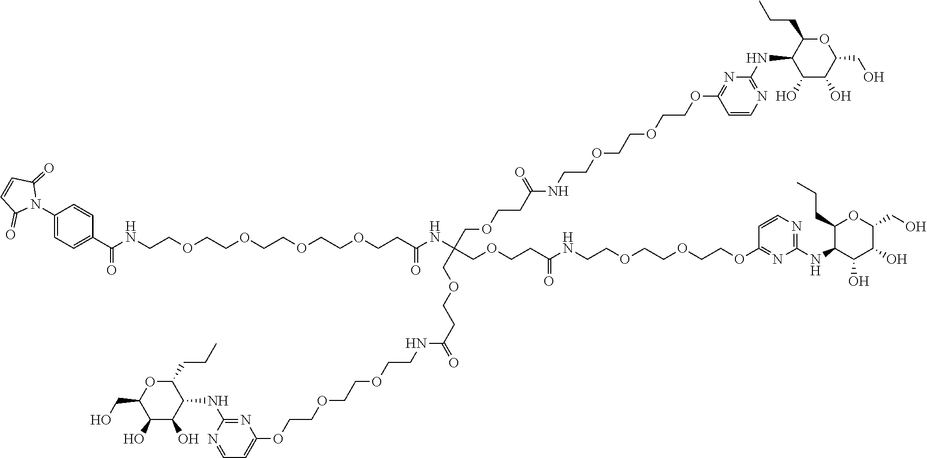 [SMILES: CCC[C@H]1O[C@H](CO)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](CO)O[C@@H]3CCC)n2)(COCCC(=O)NCCOCCOCCOc2ccnc(N[C@@H]3[C@@H](O)[C@@H](O)[C@@H](CO)O[C@@H]3CCC)n2)NC(=O)CCOCCOCCOCCOCCNC(=O)c2ccc(N3C(=O)C=CC3=O)cc2)n1] |
| 2354 | 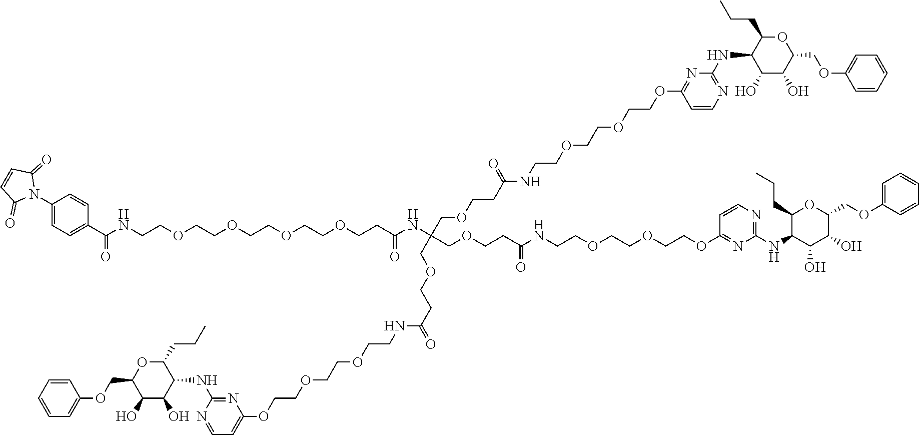 [SMILES: CCC[C@H]1O[C@H](COc2ccccc2)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](COc4ccccc4)O[C@@H]3CCC)n2)(COCCC(=O)NCCOCCOCCOc2ccnc(N[C@@H]3[C@@H](O)[C@@H](O)[C@@H](COc4ccccc4)O[C@@H]3CCC)n2)NC(=O)CCOCCOCCOCCOCCNC(=O)c2ccc(N3C(=O)C=CC3=O)cc2)n1] |
| 2355 | 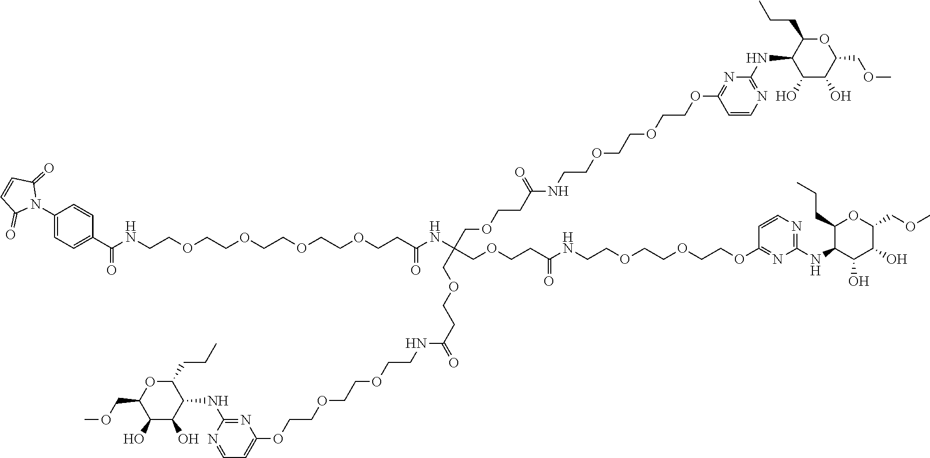 [SMILES: CCC[C@H]1O[C@H](COC)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](COC)O[C@@H]3CCC)n2)(COCCC(=O)NCCOCCOCCOc2ccnc(N[C@@H]3[C@@H](O)[C@@H](O)[C@@H](COC)O[C@@H]3CCC)n2)NC(=O)CCOCCOCCOCCOCCNC(=O)c2ccc(N3C(=O)C=CC3=O)cc2)n1] |
| 2356 | 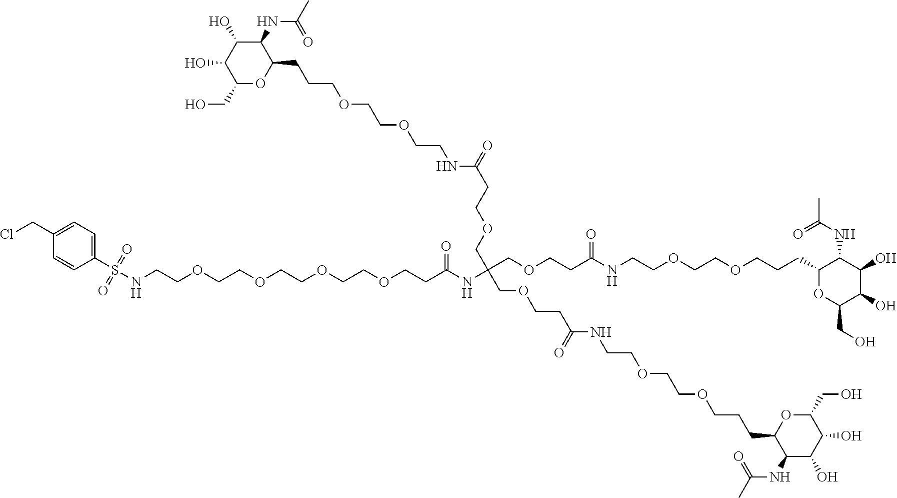 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNS(=O)(=O)c1ccc(CCl)cc1] |
| 2357 | 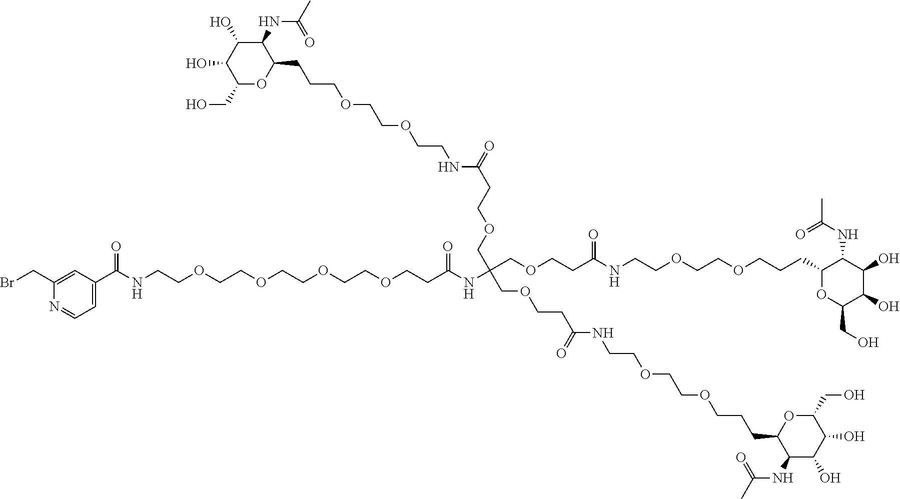 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1ccnc(CBr)c1] |
| 2358 | 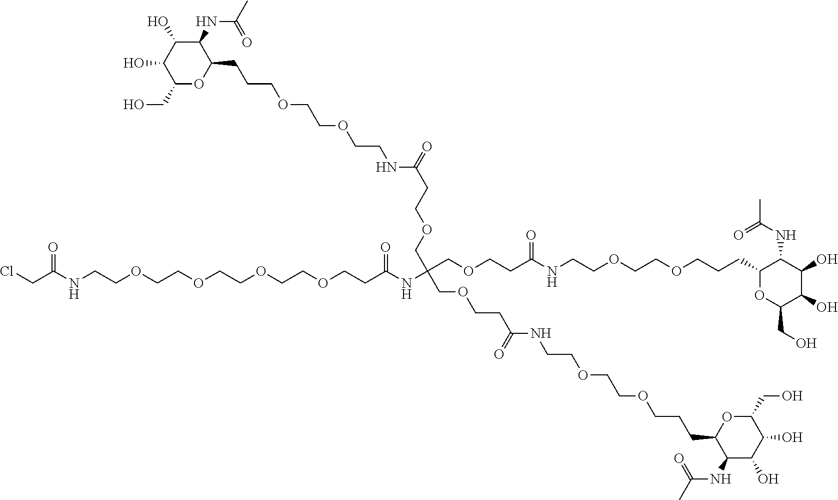 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CCl] |
| 2359 | 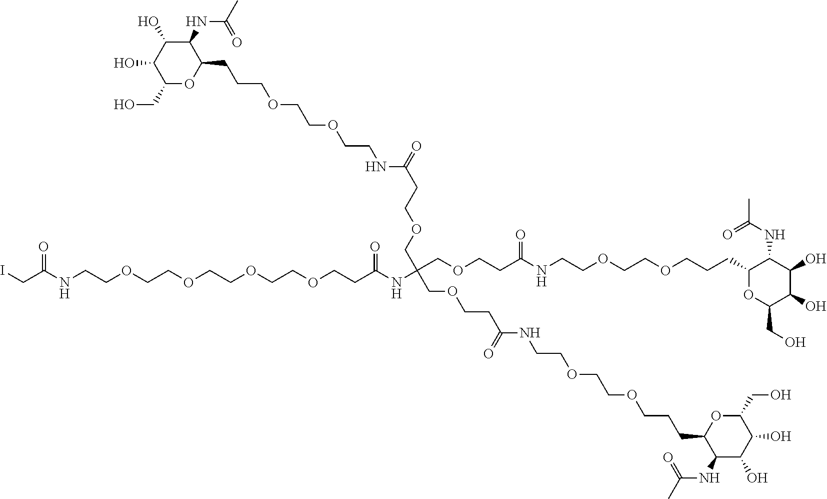 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CI] |
| 2360 | 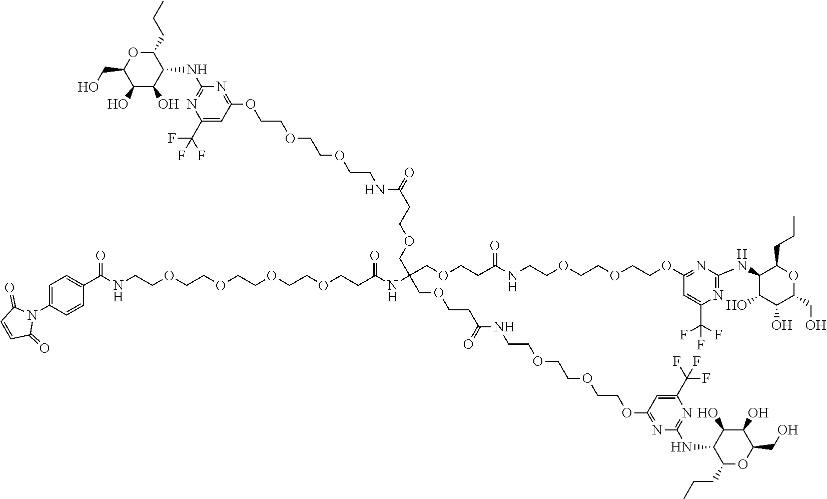 [SMILES: CCC[C@H]1O[C@H](CO)[C@H](O)[C@H](O)[C@H]1Nc1nc(OCCOCCOCCNC(=O)CCOCC(COCCC(=O)NCCOCCOCCOc2cc(C(F)(F)F)nc(N[C@@H]3[C@@H](O)[C@@H](O)[C@@H](CO)O[C@@H]3CCC)n2)(COCCC(=O)NCCOCCOCCOc2cc(C(F)(F)F)nc(N[C@@H]3[C@@H](O)[C@@H](O)[C@@H](CO)O[C@@H]3CCC)n2)NC(=O)CCOCCOCCOCCOCCNC(=O)c2ccc(N3C(=O)C=CC3=O)cc2)cc(C(F)(F)F)n1] |
| 2361 | 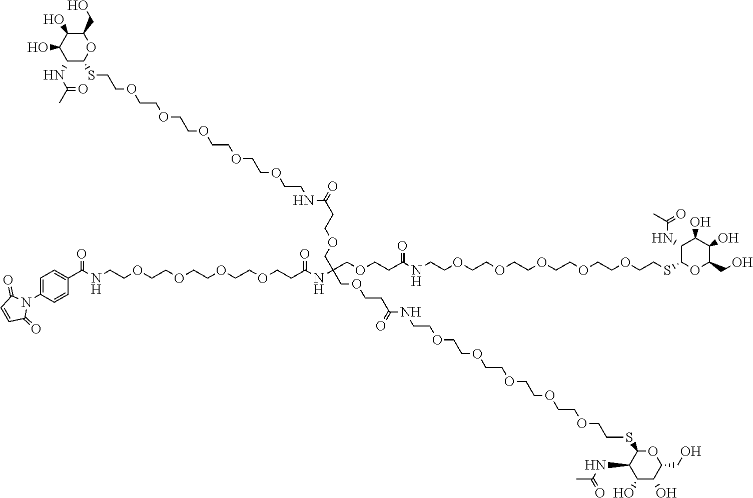 [SMILES: CC(=O)N[C@@H]1[C@@H](O)[C@@H](O)[C@@H](CO)O[C@@H]1SCCOCCOCCOCCOCCOCCNC(=O)CCOCC(COCCC(=O)NCCOCCOCCOCCOCCOCCS[C@H]1O[C@H](CO)[C@H](O)[C@H](O)[C@H]1NC(C)=O)(COCCC(=O)NCCOCCOCCOCCOCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2362 | 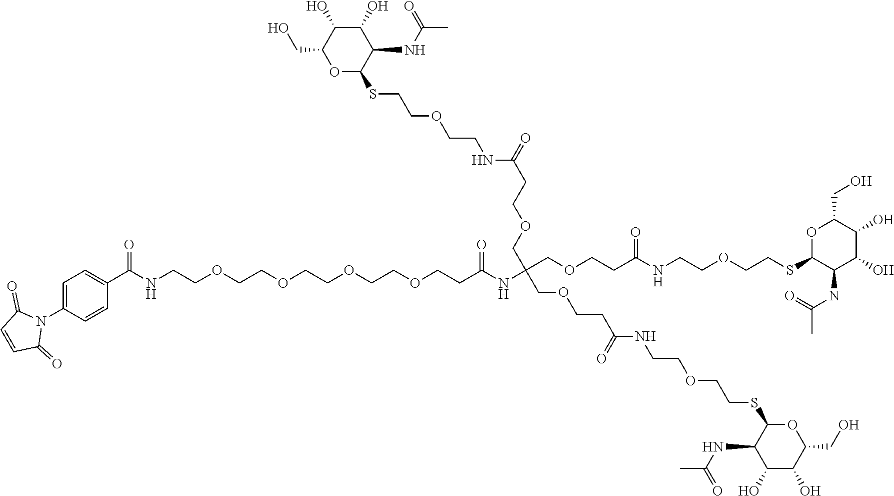 [SMILES: CC(=O)N[C@@H]1[C@@H](O)[C@@H](O)[C@@H](CO)O[C@@H]1SCCOCCNC(=O)CCOCC(COCCC(=O)NCCOCCS[C@H]1O[C@H](CO)[C@H](O)[C@H](O)[C@H]1NC(C)=O)(COCCC(=O)NCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2363 | 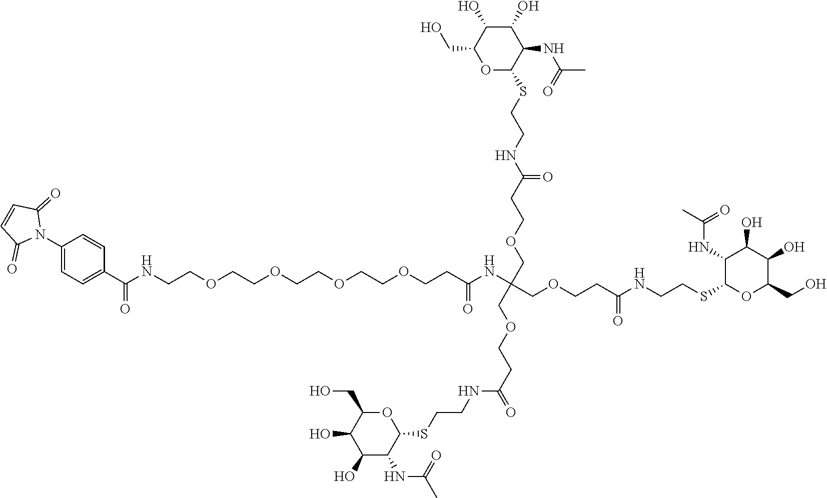 [SMILES: CC(=O)N[C@@H]1[C@@H](O)[C@@H](O)[C@@H](CO)O[C@@H]1SCCNC(=O)CCOCC(COCCC(=O)NCCS[C@H]1O[C@H](CO)[C@H](O)[C@H](O)[C@H]1NC(C)=O)(COCCC(=O)NCCS[C@@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2364 | 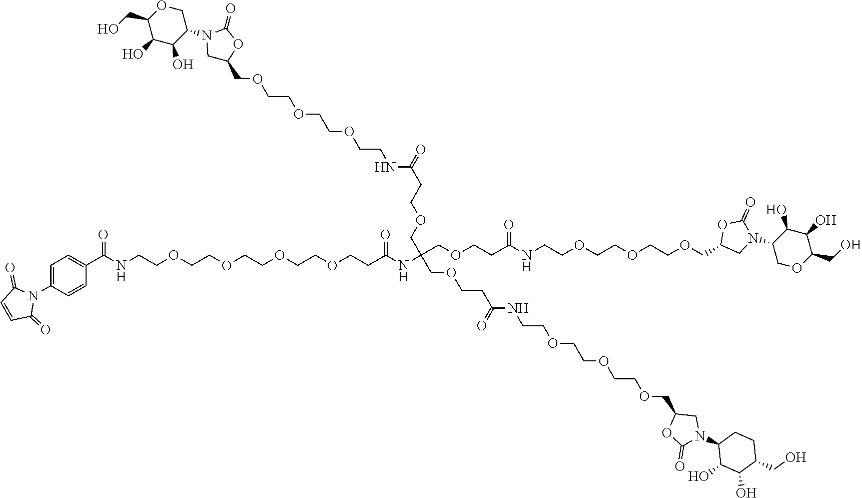 [SMILES: O=C(CCOCC(COCCC(=O)NCCOCCOCCOC[C@H]1CN([C@H]2CO[C@H](CO)[C@H](O)[C@@H]2O)C(=O)O1)(COCCC(=O)NCCOCCOCCOC[C@H]1CN([C@H]2CC[C@H](CO)[C@H](O)[C@@H]2O)C(=O)O1)NC(=O)CCOCCOCCOCCOCCNC(=O)c1ccc(N2C(=O)C=CC2=O)cc1)NCCOCCOCCOC[C@H]1CN([C@H]2CO[C@H](CO)[C@H](O)[C@@H]2O)C(=O)O1] |
| 2365 | 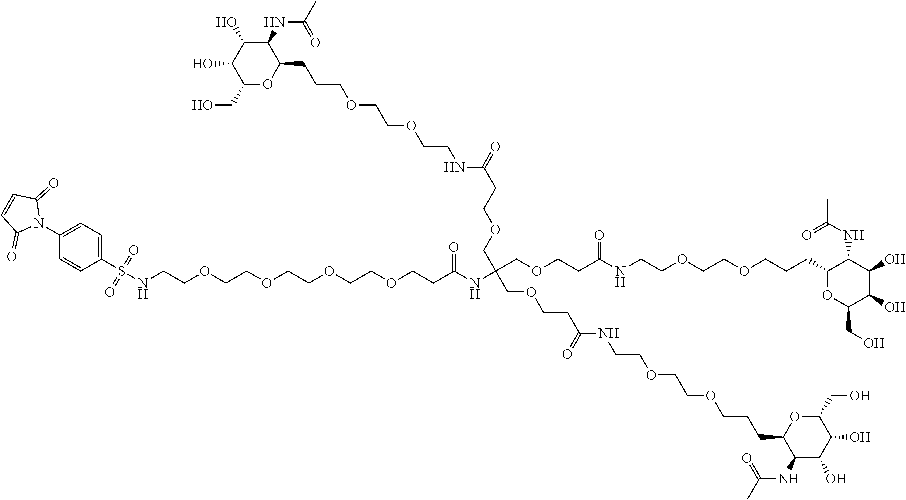 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNS(=O)(=O)c1ccc(N2C(=O)C=CC2=O)cc1] |
| 2366 | 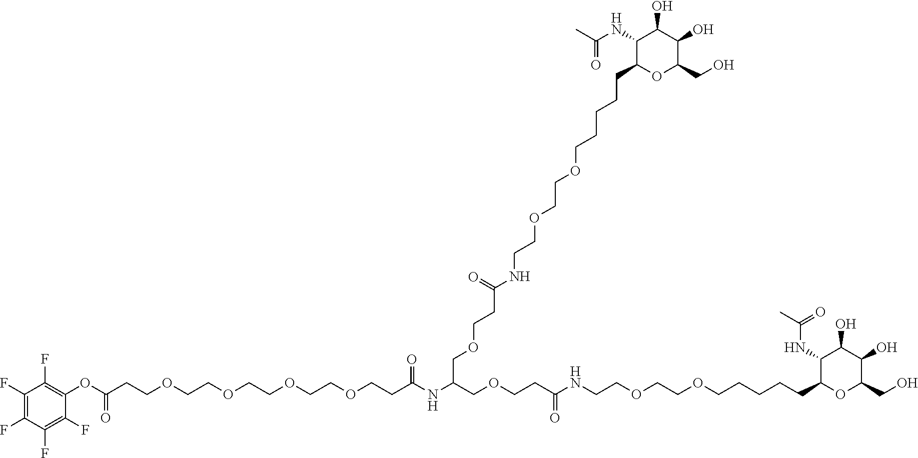 [SMILES: CC(=O)N[C@@H]1[C@@H](O)[C@@H](O)[C@@H](CO)O[C@H]1CCCCCOCCOCCNC(=O)CCOCC(COCCC(=O)NCCOCCOCCCCC[C@@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2367 | 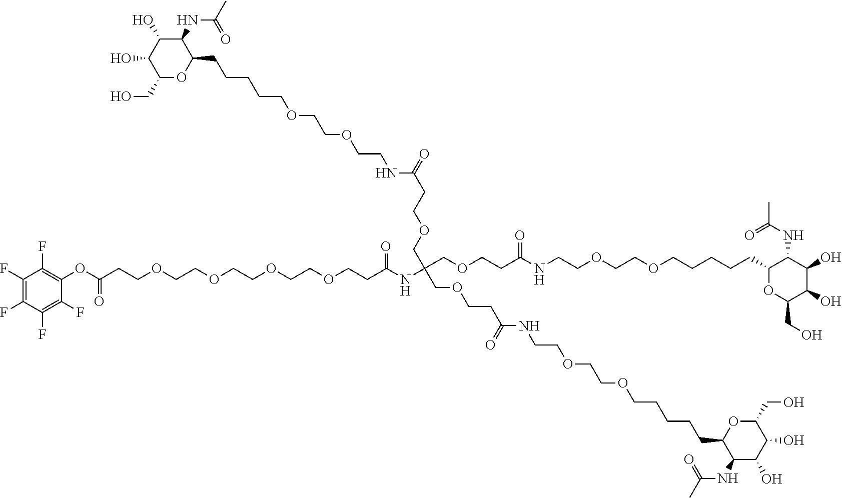 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2368 | 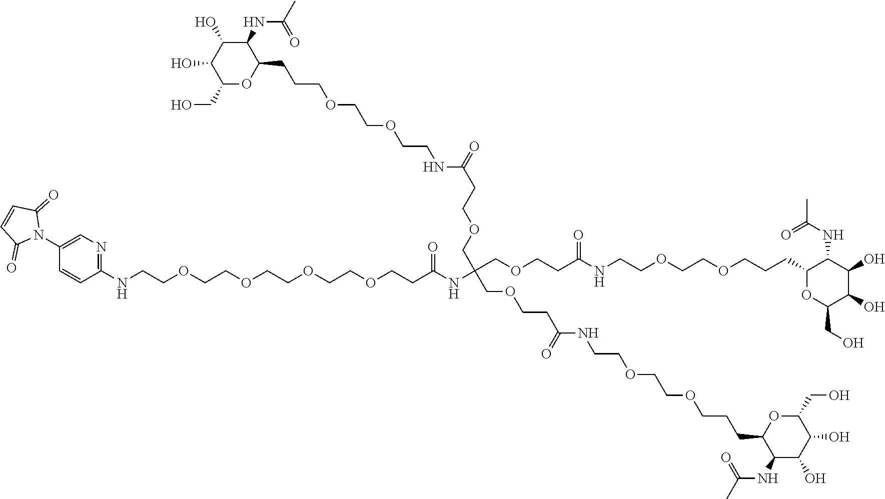 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNc1ccc(N2C(=O)C=CC2=O)cn1] |
| 2369 | 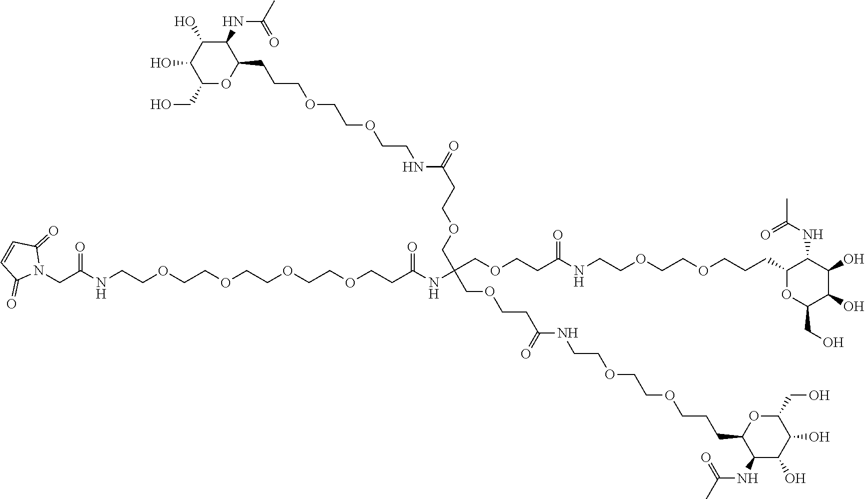 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CN1C(=O)C=CC1=O] |
| 2370 | 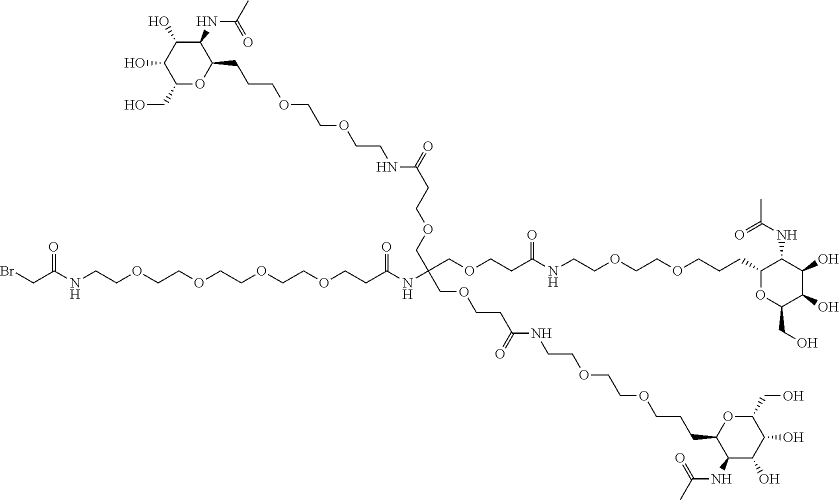 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CBr] |
| 2371 | 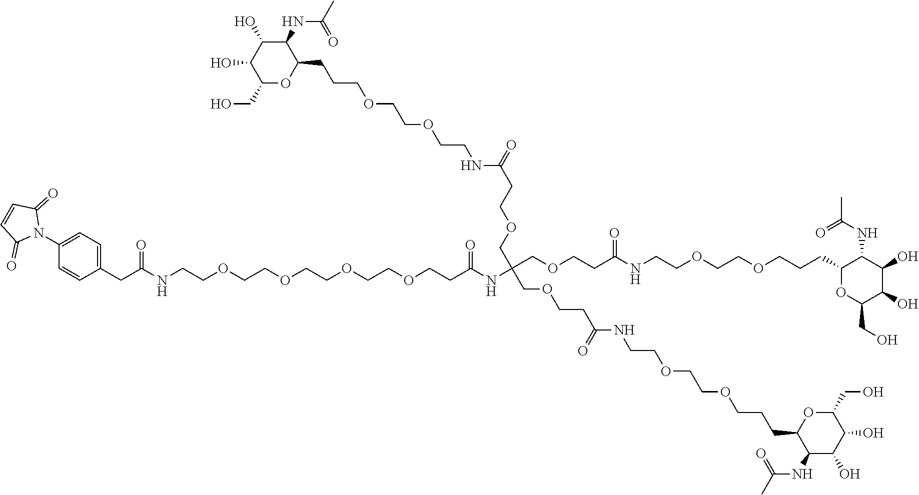 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)Cc1ccc(N2C(=O)C=CC2=O)cc1] |
| 2372 | 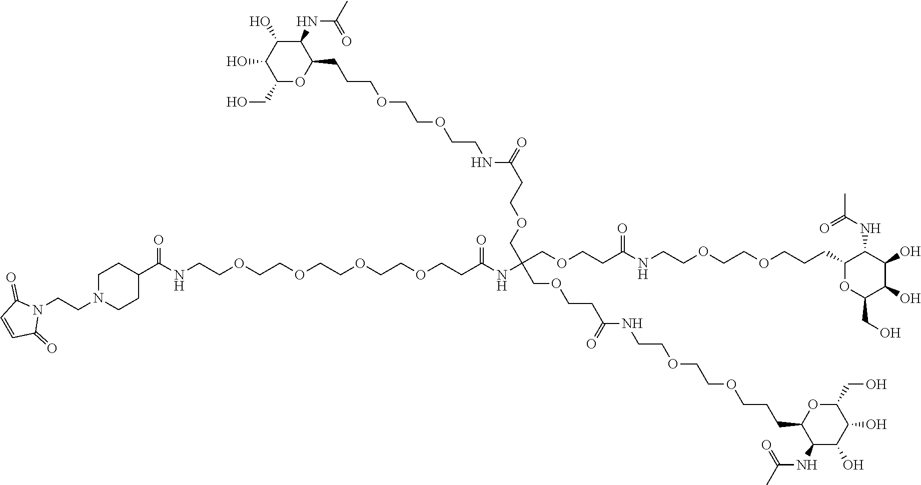 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)C1CCN(CCN2C(=O)C=CC2=O)CC1] |
| 2373 | 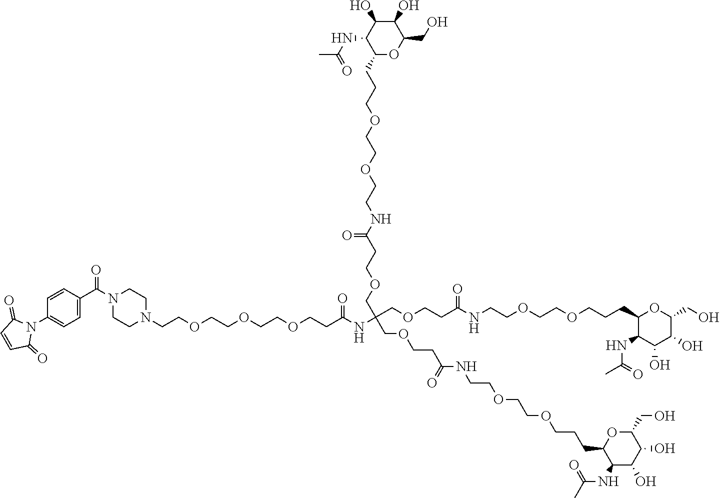 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCN1CCN(C(=O)c2ccc(N3C(=O)C=CC3=O)cc2)CC1] |
| 2374 | 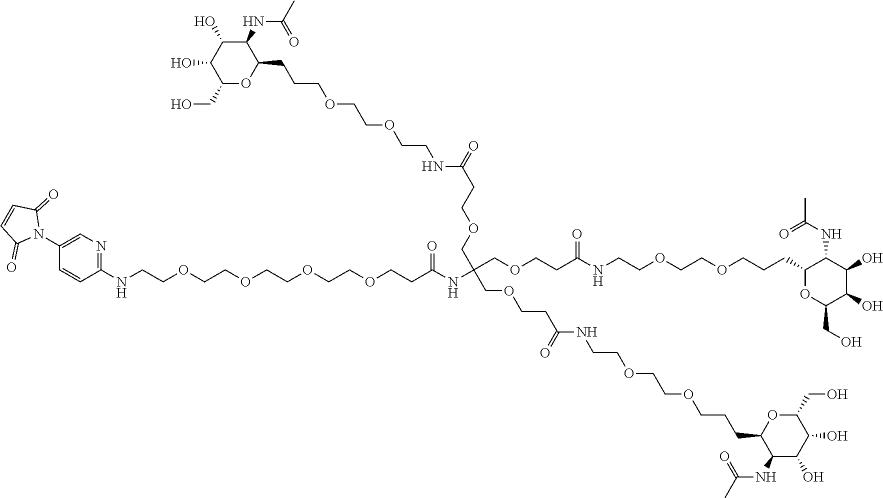 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNc1ccc(N2C(=O)C=CC2=O)cn1] |
| 2375 | 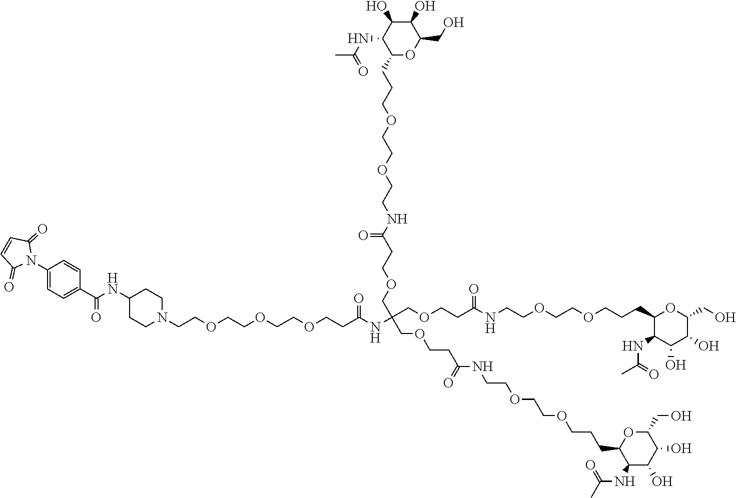 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCN1CCC(NC(=O)c2ccc(N3C(=O)C=CC3=O)cc2)CC1] |
| 2376 | 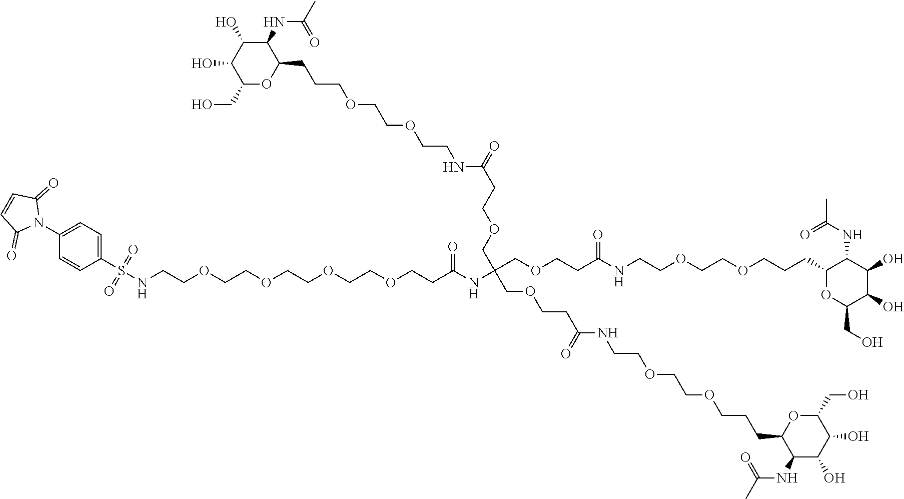 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNS(=O)(=O)c1ccc(N2C(=O)C=CC2=O)cc1] |
| 2377 | 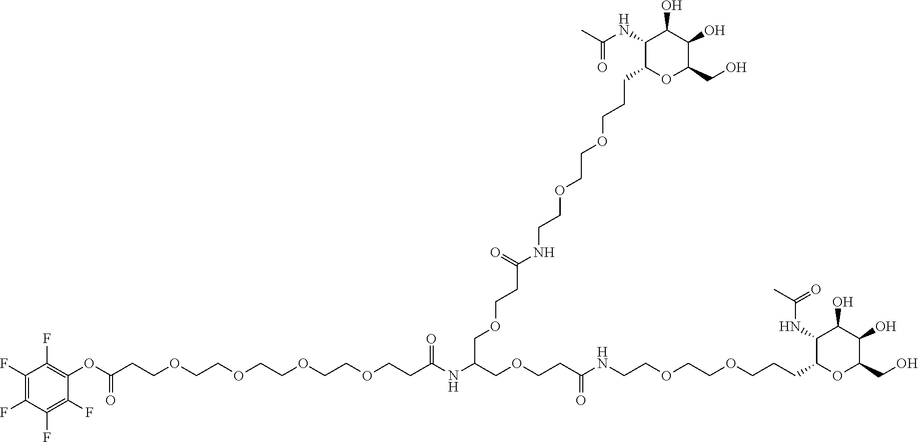 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2378 | 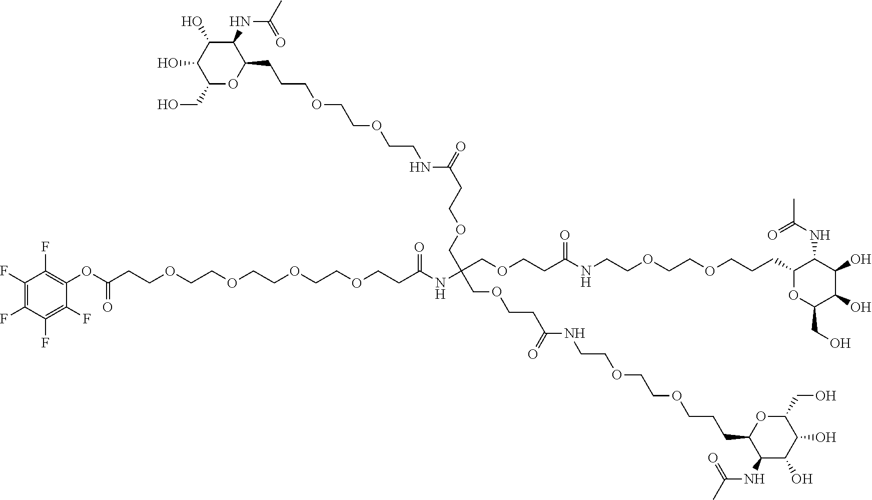 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2379 | 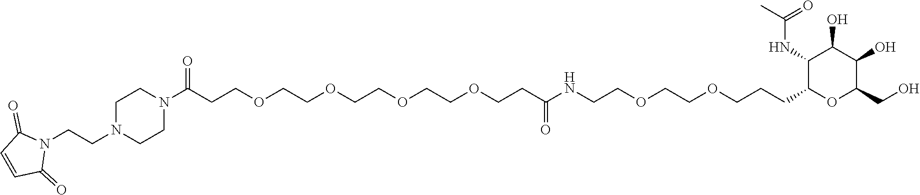 [SMILES: CC(=O)N[C@@H]1[C@@H](O)[C@@H](O)[C@@H](CO)O[C@@H]1CCCOCCOCCNC(=O)CCOCCOCCOCCOCCC(=O)N1CCN(CCN2C(=O)C=CC2=O)CC1] |
| 2380 | 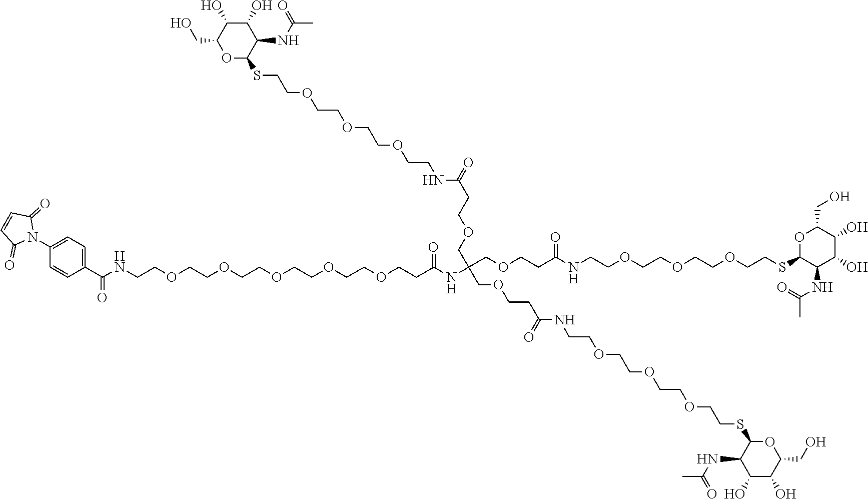 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCOCCOCCNC(=O)c1ccc(N2C(=O)C=CC2=O)cc1] |
| 2381 | 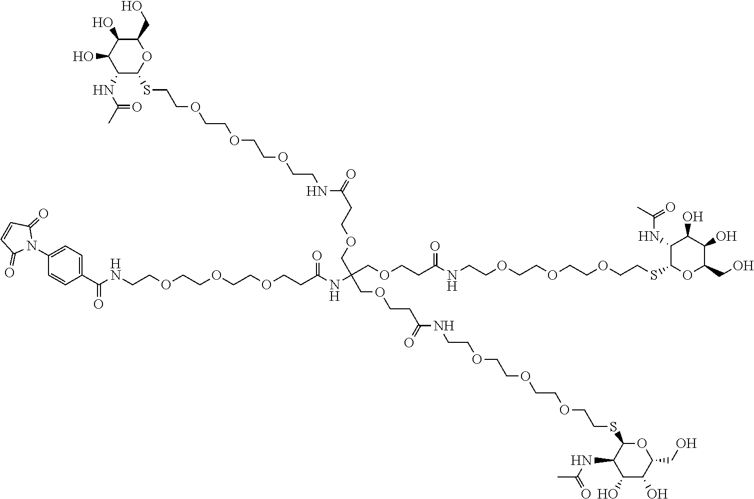 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2382 | 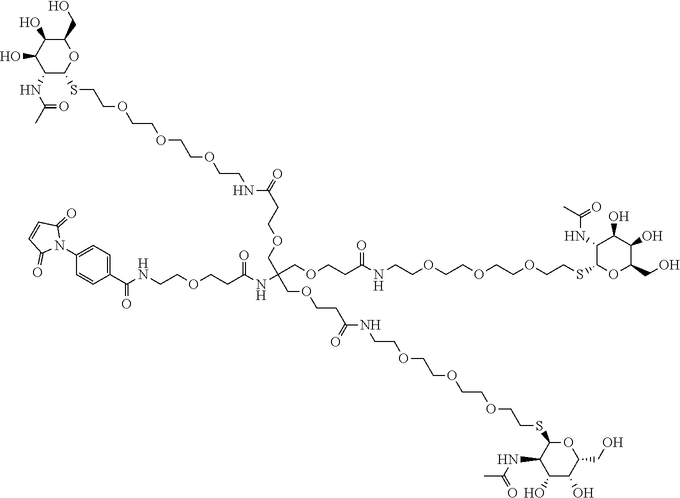 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCNC(=O)c1ccc(N2C(=O)C=CC2=O)cc1] |
| 2383 | 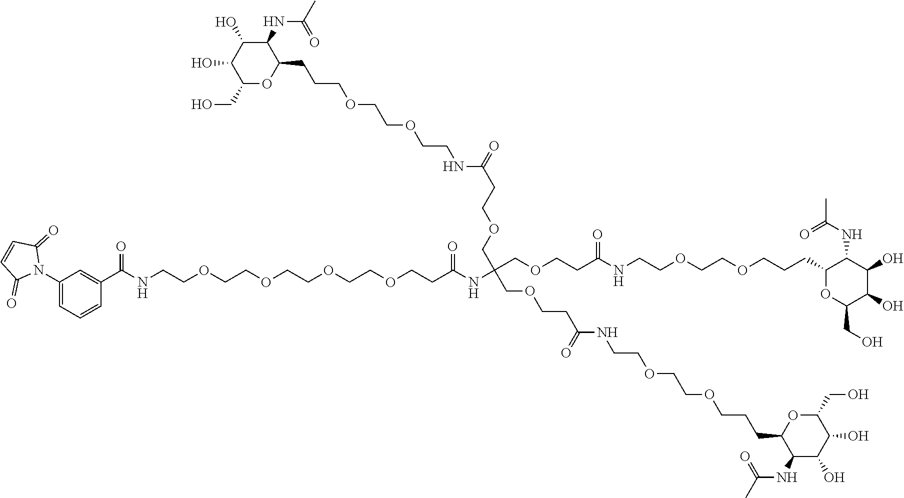 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1cccc(N2C(=O)C=CC2=O)c1] |
| 2384 | 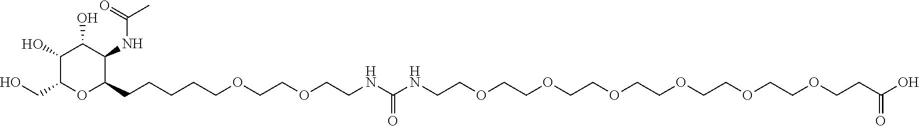 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)O] |
| 2385 | 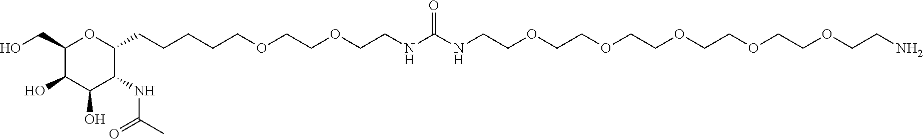 [SMILES: CC(=O)N[C@@H]1[C@@H](O)[C@@H](O)[C@@H](CO)O[C@@H]1CCCCCOCCOCCNC(=O)NCCOCCOCCOCCOCCOCCN] |
| 2386 | 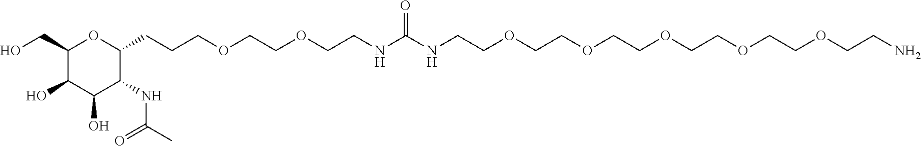 [SMILES: CC(=O)N[C@@H]1[C@@H](O)[C@@H](O)[C@@H](CO)O[C@@H]1CCCOCCOCCNC(=O)NCCOCCOCCOCCOCCOCCN] |
| 2401-C | 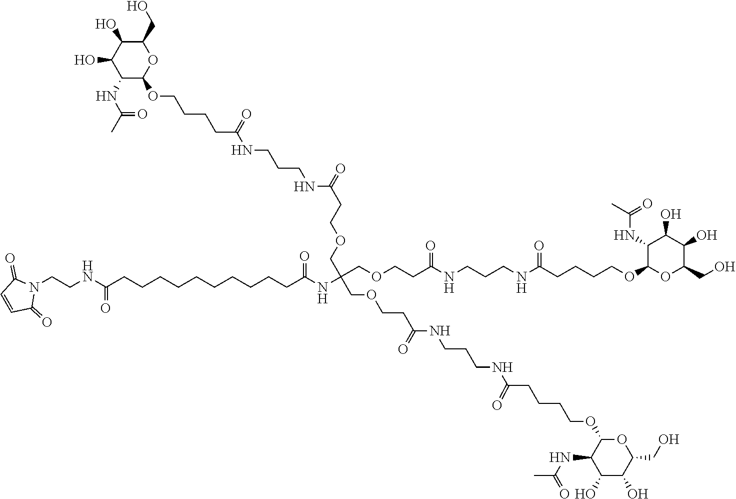 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| 2402-C | 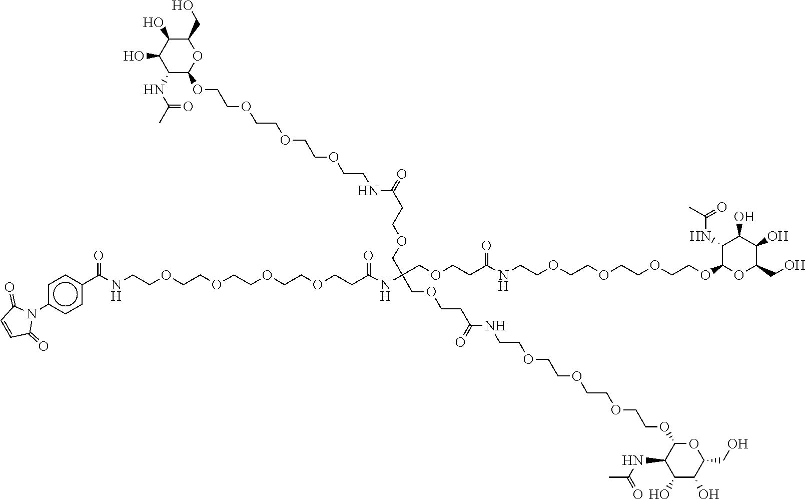 |
| 2403 | 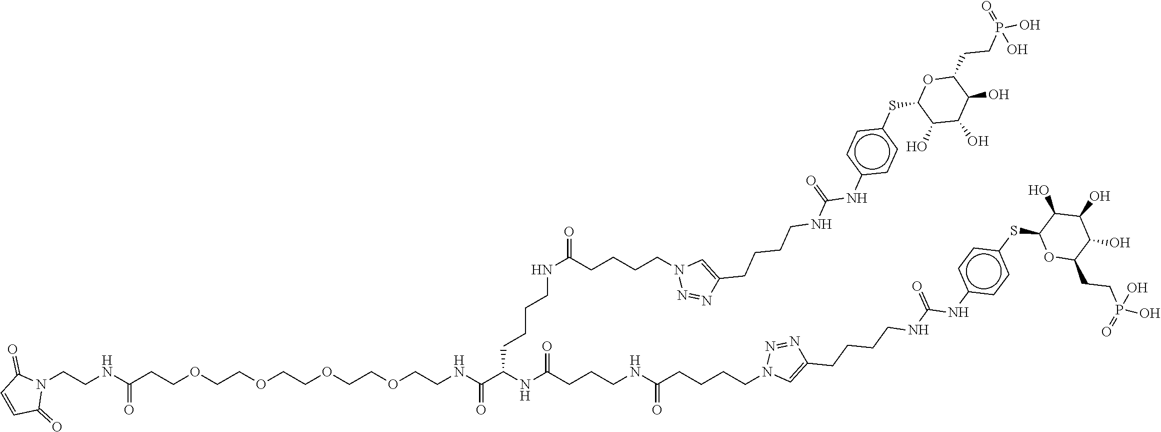 |

The present disclosure is meant to encompass stereoisomers of any one of the compounds described herein. In some instance, the compound includes an enantiomer of the D-N-acetylgalactosamine (GalNAc), or an analog or derivative of GalNAc.

Exemplary CI-M6PR-Binding Compounds with Chemoselective Ligation Group for Preparing Conjugates

Exemplary M6PR binding moieties, X, of formula (I)-(XIII) which can be utilized in the preparation of compounds and conjugates of this disclosure are shown in Table 23.

TABLE 23
Exemplary M6PR binding moieties, X

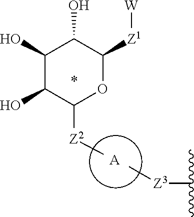

| # | W | Z¹ | Z² | * | A | Z³ |
|---|---|---|---|---|---|---|
| X1 | —P(═O)(OH)₂ | —CH₂CH₂— | —O— | alpha | 1,4-phenylene | —NHCO— |
| X2 | —P(═O)(OH)₂ | —CH₂CH₂— | —O— | alpha | 1,4-phenylene | —NHC(═S)NH— |
| X3 | —P(═O)(OH)₂ | —CH₂CH₂— | —O— | alpha | 1,4-phenylene | —NHC(═O)NH— |
| X4 | —P(═O)(OH)₂ | —CH₂CH₂— | —O— | alpha | 1,4-phenylene | —CH₂— |
| X5 | —P(═O)(OH)₂ | —CH₂CH₂— | —O— | alpha | 1,4-phenylene | —OCH₂— |
| X6 | —P(═O)(OH)₂ | —CH₂CH₂— | —O— | alpha | 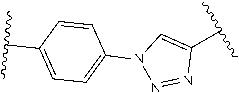 [SMILES: *c1ccc(-n2cc(*)nn2)cc1] | — |
| X7 | —P(═O)(OH)₂ | —CH₂CH₂— | —O— | alpha | 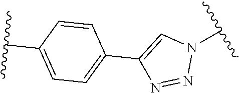 [SMILES: *c1ccc(-c2cn(*)nn2)cc1] | — |
| X8 | —P(═O)(OH)₂ | —CH₂CH₂— | —O— | alpha | 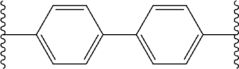 [SMILES: *c1ccc(-c2ccc(*)cc2)cc1] | — |
| X9 | —P(═O)(OH)₂ | —CH₂CH₂— | —O— | alpha | 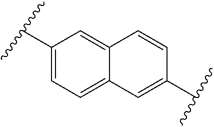 [SMILES: *c1ccc2cc(*)ccc2c1] | —NHCO— |
| X10 | —P(═O)(OH)₂ | —CH₂CH₂— | —O— | alpha | 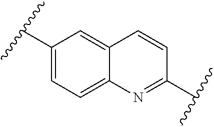 [SMILES: *c1ccc2nc(*)ccc2c1] | —NHCO— |
| X11 | —P(═O)(OH)₂ | —CH₂CH₂— | —S— | alpha | 1,4-phenylene | —NHC(═O)NH— |
| X12 | —P(═O)(OH)₂ | —CH₂CH₂— | —O— | alpha | 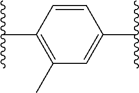 [SMILES: *c1ccc(*)c(C)c1] | —NHC(═O)NH— |
| X13 | —P(═O)(OH)₂ | —CH₂CH₂— | —O— | alpha | 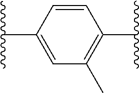 [SMILES: *c1ccc(*)c(C)c1] | —NHC(═O)NH— |
| X14 | —P(═O)(OH)₂ | —CH₂CH₂— | —O— | alpha | 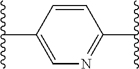 [SMILES: *c1ccc(*)nc1] | —NHC(═O)NH— |
| X15 | —P(═O)(OH)₂ | —CH₂CH₂— | —O— | alpha | 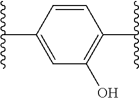 [SMILES: *c1ccc(*)c(O)c1] | —NHC(═O)NH— |
| X16 | —P(═O)(OH)₂ | —CH₂CH₂— | —O— | alpha | 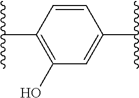 [SMILES: *c1ccc(*)c(O)c1] | —NHC(═O)NH— |
| X17 | —P(═O)(OH)₂ | —CH₂CF₂— | —O— | alpha | 1,4-phenylene | —NHCO— |
| X18 | —P(═O)(OH)₂ | —CH₂CF₂— | —O— | alpha | 1,4-phenylene | —NHC(═S)NH— |
| X19 | —COOH | —CH₂CH₂— | —O— | alpha | 1,4-phenylene | —NHC(═S)NH— |
| X20 | —COOH | —CH═CH— | —O— | alpha | 1,4-phenylene | —NHC(═O)NH— |
| X21 | —CH(COOH)₂ | —CH₂— | —O— | alpha | 1,4-phenylene | —NHCO— |
| X22 | —CH(COOH)₂ | —CH₂— | —O— | alpha | 1,4-phenylene | —NHC(═S)NH— |
| X23 | —CH(COOH)₂ | —CH₂— | —O— | alpha | 1,4-phenylene | —NHC(═O)NH— |
| X24 | —SO₃H | —CH₂CH₂— | —O— | alpha | 1,4-phenylene | —NHCO— |
| X25 | —SO₃H | —CH₂CH₂— | —O— | alpha | 1,4-phenylene | —NHC(═S)NH— |
| X26 | —P(═O)(OH)₂ | —CH₂CH₂— | —CH₂— | alpha | 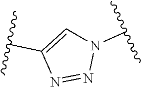 [SMILES: *c1cn(*)nn1] | — |
| X27 | —P(═O)(OH)₂ | —CH₂CH₂— | —CH₂— | alpha | 1,4-phenylene | —NHC(═O)NH— |
| X28 | —NHC(═O)CO₂H | —CH₂— | —O— | alpha | 1,4-phenylene | —NHC(═O)NH— |
| X29 | 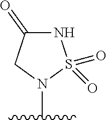 [SMILES: *N1CC(=O)NS1(=O)=O] | —CH₂— | —O— | alpha | 1,4-phenylene | —NHC(═O)NH— |
| X30 | —NHSO₂Me | —CH₂— | —O— | alpha | 1,4-phenylene | —NHC(═O)NH— |
| X31 | —NHSO₂NH₂ | —CH₂— | —O— | alpha | 1,4-phenylene | —NHC(═O)NH— |
| X32 | —NHC(═O)NHSO₂Me | —CH₂— | —O— | alpha | 1,4-phenylene | —NHC(═O)NH— |
| X33 | —NHSO₃H | —CH₂— | —O— | alpha | 1,4-phenylene | —NHC(═O)NH— |
| X34 | —P(═O)(OH)₂ | —CH₂CH₂— | —O— | alpha | 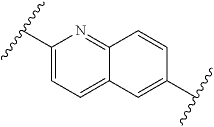 [SMILES: *c1ccc2nc(*)ccc2c1] | —NHC(═O)NH— |
| X35 | —P(═O)(OH)₂ | —CH₂CH₂— | —O— | alpha | 1,4-phenylene | —CONH— |
| X36 | —P(═O)(OH)₂ | —CH₂CH₂— | —O— | alpha | 1,4-phenylene | —NHSO₂— |
| X37 | —P(═O)(OH)₂ | —CH₂CH₂— | —O— | alpha | 1,4-phenylene | —SO₂NH— |
| X38 | —P(═O)(OH)₂ | —CH₂— | —O— | alpha | 1,4-phenylene | —NHC(═O)NH— |
| X39 | —P(═O)(OH)₂ | —CH₂CH₂— | —CF₂— | alpha | 1,4-phenylene | —NHC(═O)NH— |
| X40 | —P(═O)(OH)₂ | —CH₂CH₂— | —CF₂— | alpha | 1,4-phenylene | —NHC(═S)NH— |
| X41 | —P(═O)(OH)₂ | —CH₂CH₂— | —CH₂— | alpha | 1,4-phenylene | —NHC(═S)NH— |
| X42 | —P(═O)(OH)₂ | —CH₂CH₂— | —S— | alpha | 1,4-phenylene | —NHC(═S)NH— |
| X43 | —P(═O)(OH)₂ | —CH₂CH₂— | —S— | alpha | 1,4-phenylene | —NHCO— |
| X44 | —P(═O)(OH)₂ | —CH₂CH₂— | —S— | alpha | 1,4-phenylene | —CONH— |
| X45 | —P(═O)(OH)₂ | —CH₂CH₂— | —S— | alpha | 1,4-phenylene | —NHSO₂— |
| X46 | —P(═O)(OH)₂ | —CH₂CH₂— | —S— | alpha | 1,4-phenylene | —SO₂NH— |
| X47 | —P(═O)(OH)₂ | —CH₂CH₂— | —S— | alpha | 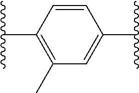 [SMILES: *c1ccc(*)c(C)c1] | —NHC(═O)NH— |
| X48 | —P(═O)(OH)₂ | —CH₂CH₂— | —S— | alpha | 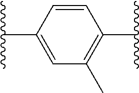 [SMILES: *c1ccc(*)c(C)c1] | —NHC(═O)NH— |
| X49 | —P(═O)(OH)₂ | —CH₂CH₂— | —S— | alpha | 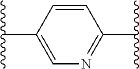 [SMILES: *c1ccc(*)nc1] | —NHC(═O)NH— |
| X50 | —P(═O)(OH)₂ | —CH₂CH₂— | —S— | alpha | 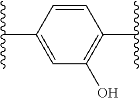 [SMILES: *c1ccc(*)c(O)c1] | —NHC(═O)NH— |
| X51 | —P(═O)(OH)₂ | —CH₂CH₂— | —S— | alpha | 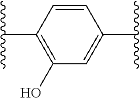 [SMILES: *c1ccc(*)c(O)c1] | —NHC(═O)NH— |

Beta configuration moieties

| | | | | | | |
|---|---|---|---|---|---|---|
| X1* | —P(═O)(OH)₂ | —CH₂CH₂— | —O— | beta | 1,4-phenylene | —NHCO— |
| X2* | —P(═O)(OH)₂ | —CH₂CH₂— | —O— | beta | 1,4-phenylene | —NHC(═S)NH— |
| X3* | —P(═O)(OH)₂ | —CH₂CH₂— | —O— | beta | 1,4-phenylene | —NHC(═O)NH— |
| X4* | —P(═O)(OH)₂ | —CH₂CH₂— | —O— | beta | 1,4-phenylene | —CH₂— |
| X5* | —P(═O)(OH)₂ | —CH₂CH₂— | —O— | beta | 1,4-phenylene | —OCH₂— |
| X6* | —P(═O)(OH)₂ | —CH₂CH₂— | —O— | beta | 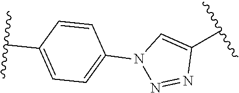 [SMILES: *c1ccc(-n2cc(*)nn2)cc1] | — |
| X7* | —P(═O)(OH)₂ | —CH₂CH₂— | —O— | beta | 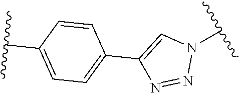 [SMILES: *c1ccc(-c2cn(*)nn2)cc1] | — |
| X8* | —P(═O)(OH)₂ | —CH₂CH₂— | —O— | beta | 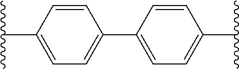 [SMILES: *c1ccc(-c2ccc(*)cc2)cc1] | — |
| X9* | —P(═O)(OH)₂ | —CH₂CH₂— | —O— | beta | 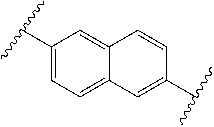 [SMILES: *c1ccc2cc(*)ccc2c1] | —NHCO— |
| X10* | —P(═O)(OH)₂ | —CH₂CH₂— | —O— | beta | 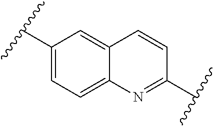 [SMILES: *c1ccc2nc(*)ccc2c1] | —NHCO— |
| X11* | —P(═O)(OH)₂ | —CH₂CH₂— | —O— | beta | 1,4-phenylene | —NHC(═O)NH— |
| X12* | —P(═O)(OH)₂ | —CH₂CH₂— | —O— | beta | 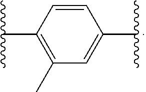 [SMILES: *c1ccc(*)c(C)c1] | —NHC(═O)NH— |
| X13* | —P(═O)(OH)₂ | —CH₂CH₂— | —O— | beta | 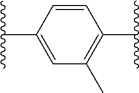 [SMILES: *c1ccc(*)c(C)c1] | —NHC(═O)NH— |
| X14* | —P(═O)(OH)₂ | —CH₂CH₂— | —O— | beta | 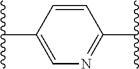 [SMILES: *c1ccc(*)nc1] | —NHC(═O)NH— |
| X15* | —P(═O)(OH)₂ | —CH₂CH₂— | —O— | beta | 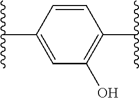 [SMILES: *c1ccc(*)c(O)c1] | —NHC(═O)NH— |
| X16* | —P(═O)(OH)₂ | —CH₂CH₂— | —O— | beta | 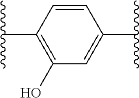 [SMILES: *c1ccc(*)c(O)c1] | —NHC(═O)NH— |
| X17* | —P(═O)(OH)₂ | —CH₂CF₂— | —O— | beta | 1,4-phenylene | —NHCO— |
| X18* | —P(═O)(OH)₂ | —CH₂CF₂— | —O— | beta | 1,4-phenylene | —NHC(═S)NH— |
| X19* | —COOH | —CH₂CH₂— | —O— | beta | 1,4-phenylene | —NHC(═S)NH— |
| X20* | —COOH | —CH═CH— | —O— | beta | 1,4-phenylene | —NHC(═O)NH— |
| X21* | —CH(COOH)₂ | —CH₂— | —O— | beta | 1,4-phenylene | —NHCO— |
| X22* | —CH(COOH)₂ | —CH₂— | —O— | beta | 1,4-phenylene | —NHC(═S)NH— |
| X23* | —CH(COOH)₂ | —CH₂— | —O— | beta | 1,4-phenylene | —NHC(═O)NH— |
| X24* | —SO₃H | —CH₂CH₂— | —O— | beta | 1,4-phenylene | —NHCO— |
| X25* | —SO₃H | —CH₂CH₂— | —O— | beta | 1,4-phenylene | —NHC(═S)NH— |
| X26* | —P(═O)(OH)₂ | —CH₂CH₂— | —CH₂— | beta | 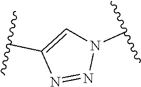 [SMILES: *c1cn(*)nn1] | — |
| X27* | —P(═O)(OH)₂ | —CH₂CH₂— | —CH₂— | beta | 1,4-phenylene | —NHC(═O)NH— |
| X28* | —NHC(═O)CO₂H | —CH₂— | —O— | beta | 1,4-phenylene | —NHC(═O)NH— |
| X29* | 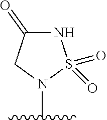 [SMILES: *N1CC(=O)NS1(=O)=O] | —CH₂— | —O— | beta | 1,4-phenylene | —NHC(═O)NH— |
| X30* | —NHSO₂Me | —CH₂— | —O— | beta | 1,4-phenylene | —NHC(═O)NH— |
| X31* | —NHSO₂NH₂ | —CH₂— | —O— | beta | 1,4-phenylene | —NHC(═O)NH— |
| X32* | —NHC(═O)NHSO₂Me | —CH₂— | —O— | beta | 1,4-phenylene | —NHC(═O)NH— |
| X33* | —NHSO₃H | —CH₂— | —O— | beta | 1,4-phenylene | —NHC(═O)NH— |
| X34* | —P(═O)(OH)₂ | —CH₂CH₂— | —O— | beta | 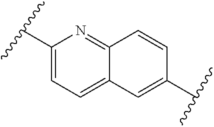 [SMILES: *c1ccc2nc(*)ccc2c1] | —NHC(═O)NH— |
| X35* | —P(═O)(OH)₂ | —CH₂CH₂— | —O— | beta | 1,4-phenylene | —CONH— |
| X36* | —P(═O)(OH)₂ | —CH₂CH₂— | —O— | beta | 1,4-phenylene | —NHSO₂— |
| X37* | —P(═O)(OH)₂ | —CH₂CH₂— | —O— | beta | 1,4-phenylene | —SO₂NH— |
| X38* | —NHSO₂CF₃ | —CH₂— | —O— | beta | 1,4-phenylene | —NHC(═O)NH— |
| X39* | —P(═O)(OH)₂ | —CH₂CH₂— | —CF₂— | beta | 1,4-phenylene | —NHC(═O)NH— |
| X40* | —P(═O)(OH)₂ | —CH₂CH₂— | —CF₂— | beta | 1,4-phenylene | —NHC(═S)NH— |
| X41* | —P(═O)(OH)₂ | —CH₂CH₂— | —CH₂— | beta | 1,4-phenylene | —NHC(═S)NH— |
| X42* | —P(═O)(OH)₂ | —CH₂CH₂— | —S— | beta | 1,4-phenylene | —NHC(═S)NH— |
| X43* | —P(═O)(OH)₂ | —CH₂CH₂— | —S— | beta | 1,4-phenylene | —NHCO— |
| X44* | —P(═O)(OH)₂ | —CH₂CH₂— | —S— | beta | 1,4-phenylene | —CONH— |
| X45* | —P(═O)(OH)₂ | —CH₂CH₂— | —S— | beta | 1,4-phenylene | —NHSO₂— |
| X46* | —P(═O)(OH)₂ | —CH₂CH₂— | —S— | beta | 1,4-phenylene | —SO₂NH— |
| X47* | —P(═O)(OH)₂ | —CH₂CH₂— | —S— | beta | 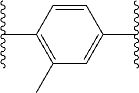 [SMILES: *c1ccc(*)c(C)c1] | —NHC(═O)NH— |
| X48* | —P(═O)(OH)₂ | —CH₂CH₂— | —S— | beta | 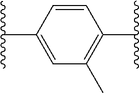 [SMILES: *c1ccc(*)c(C)c1] | —NHC(═O)NH— |
| X49* | —P(═O)(OH)₂ | —CH₂CH₂— | —S— | beta | 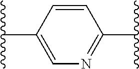 [SMILES: *c1ccc(*)nc1] | —NHC(═O)NH— |
| X50* | —P(═O)(OH)₂ | —CH₂CH₂— | —S— | beta | 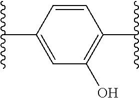 [SMILES: *c1ccc(*)c(O)c1] | —NHC(═O)NH— |
| X51* | —P(═O)(OH)₂ | —CH₂CH₂— | —S— | beta | 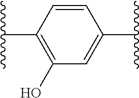 [SMILES: *c1ccc(*)c(O)c1] | —NHC(═O)NH— |

alpha refers to the following configuration: 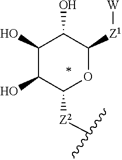
*beta refers to the following configuration:

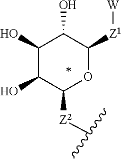

Exemplary synthons or synthetic precursors which can be utilized in the preparation of compounds of this disclosure to incorporate a desired M6PR binding moiety of interest are shown in Table 24. It is understood that alternative synthons, including homologs and analogs of the ones shown in Table 24 are possible depending on the M6PR binding moiety and linker that is selected. It is understood that the synthons of Table 24 can include structural precursors of linking moiety Z³, and a structural element that becomes part of the linker (L) in the compounds and conjugates of this disclosure. It is understood that based on the exemplary synthetic precursors of Table 24, synthons corresponding to any of the M6PR binding moieties of Table 23 can be utilized to prepare compounds of this disclosure.

TABLE 24
Exemplary Synthetic precursors for M6PR binding moieties

| Exemplary M6PR binding moiety (X) | | Exemplary Synthetic precursor(s) |
|---|---|---|
| # | Structure | Structure |
| X1 | 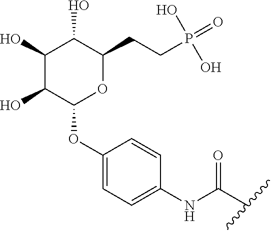 [SMILES: *C(=O)Nc1ccc(O[C@H]2O[C@H](CCP(=O)(O)O)[C@@H](O)[C@H](O)[C@@H]2O)cc1] | 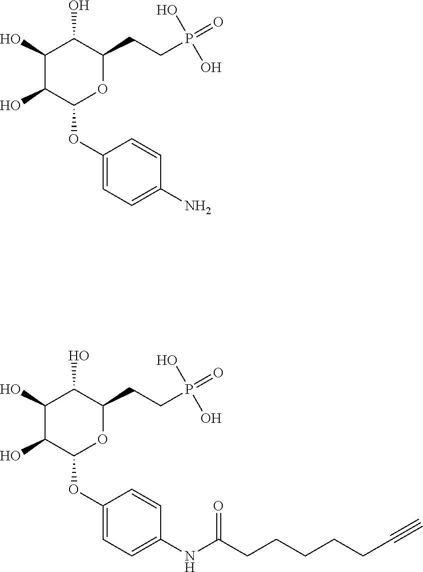 [SMILES: Nc1ccc(O[C@H]2O[C@H](CCP(=O)(O)O)[C@@H](O)[C@H](O)[C@@H]2O)cc1 ; C#CCCCCCC(=O)Nc1ccc(O[C@H]2O[C@H](CCP(=O)(O)O)[C@@H](O)[C@H](O)[C@@H]2O)cc1] |
| X2 | 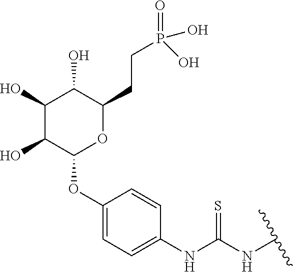 [SMILES: *NC(=S)Nc1ccc(O[C@H]2O[C@H](CCP(=O)(O)O)[C@@H](O)[C@H](O)[C@@H]2O)cc1] | 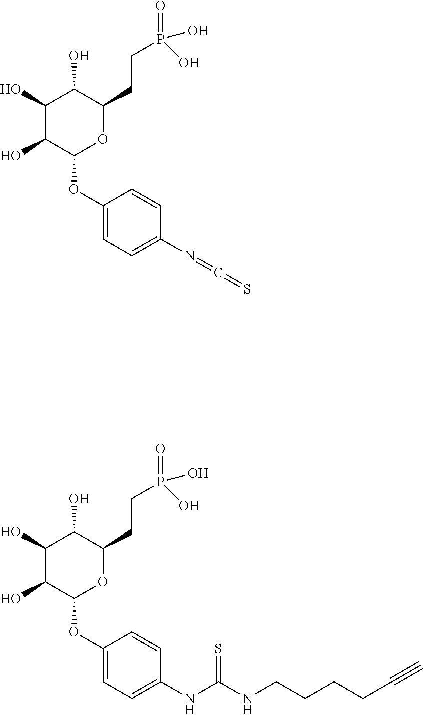 [SMILES: O=P(O)(O)CC[C@H]1O[C@H](Oc2ccc(N=C=S)cc2)[C@@H](O)[C@@H](O)[C@@H]1O ; C#CCCCCNC(=S)Nc1ccc(O[C@H]2O[C@H](CCP(=O)(O)O)[C@@H](O)[C@H](O)[C@@H]2O)cc1] |
| X3 | 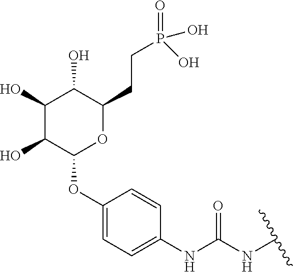 [SMILES: *NC(=O)Nc1ccc(O[C@H]2O[C@H](CCP(=O)(O)O)[C@@H](O)[C@H](O)[C@@H]2O)cc1] | 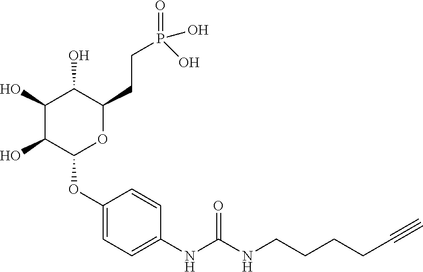 [SMILES: C#CCCCCNC(=O)Nc1ccc(O[C@H]2O[C@H](CCP(=O)(O)O)[C@@H](O)[C@H](O)[C@@H]2O)cc1] |
| X4 | 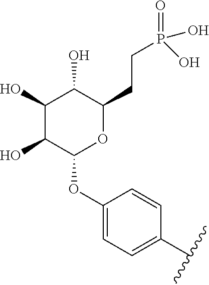 [SMILES: *c1ccc(O[C@H]2O[C@H](CCP(=O)(O)O)[C@@H](O)[C@H](O)[C@@H]2O)cc1] | 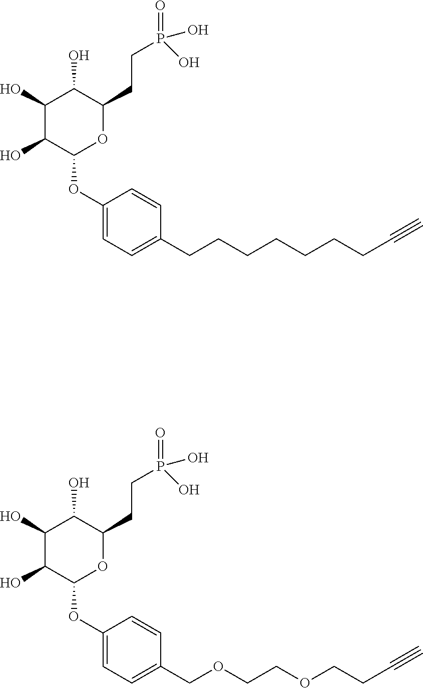 [SMILES: C#CCCCCCCCc1ccc(O[C@H]2O[C@H](CCP(=O)(O)O)[C@@H](O)[C@H](O)[C@@H]2O)cc1 ; C#CCCOCCOCc1ccc(O[C@H]2O[C@H](CCP(=O)(O)O)[C@@H](O)[C@H](O)[C@@H]2O)cc1] |
| X5 | 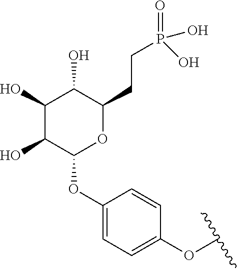 [SMILES: *Oc1ccc(O[C@H]2O[C@H](CCP(=O)(O)O)[C@@H](O)[C@H](O)[C@@H]2O)cc1] | 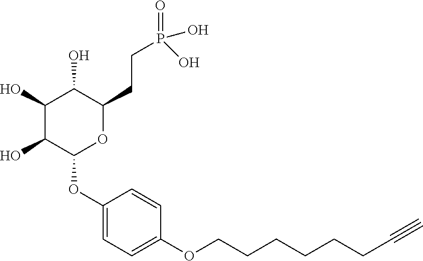 [SMILES: C#CCCCCCCOc1ccc(O[C@H]2O[C@H](CCP(=O)(O)O)[C@@H](O)[C@H](O)[C@@H]2O)cc1] |
| X6 | 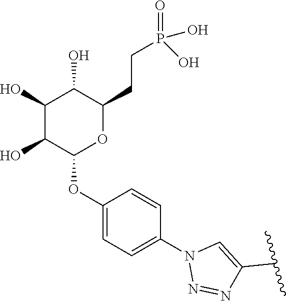 [SMILES: *c1cn(-c2ccc(O[C@H]3O[C@H](CCP(=O)(O)O)[C@@H](O)[C@H](O)[C@@H]3O)cc2)nn1] | 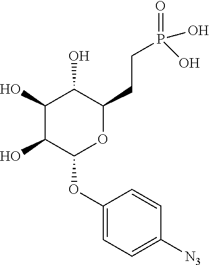 [SMILES: [N-]=[N+]=Nc1ccc(O[C@H]2O[C@H](CCP(=O)(O)O)[C@@H](O)[C@H](O)[C@@H]2O)cc1] |
| X7 | 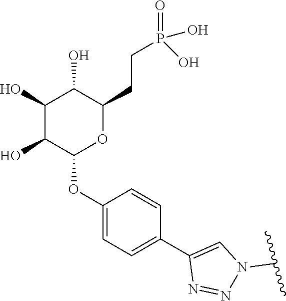 [SMILES: *n1cc(-c2ccc(O[C@H]3O[C@H](CCP(=O)(O)O)[C@@H](O)[C@H](O)[C@@H]3O)cc2)nn1] | 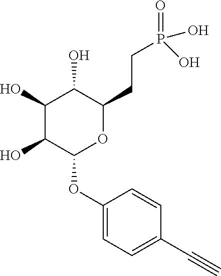 [SMILES: C#Cc1ccc(O[C@H]2O[C@H](CCP(=O)(O)O)[C@@H](O)[C@H](O)[C@@H]2O)cc1] |
| X8 | 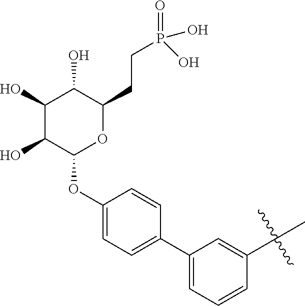 [SMILES: *c1cccc(-c2ccc(O[C@H]3O[C@H](CCP(=O)(O)O)[C@@H](O)[C@H](O)[C@@H]3O)cc2)c1] | 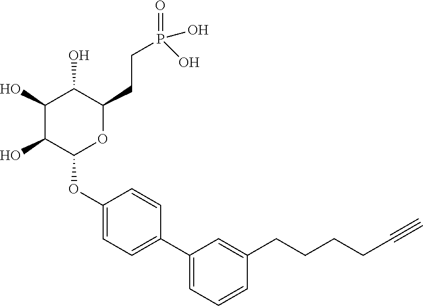 [SMILES: C#CCCCCc1cccc(-c2ccc(O[C@H]3O[C@H](CCP(=O)(O)O)[C@@H](O)[C@H](O)[C@@H]3O)cc2)c1] |
| X9 | 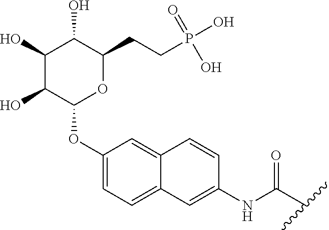 [SMILES: *C(=O)Nc1ccc2cc(O[C@H]3O[C@H](CCP(=O)(O)O)[C@@H](O)[C@H](O)[C@@H]3O)ccc2c1] | 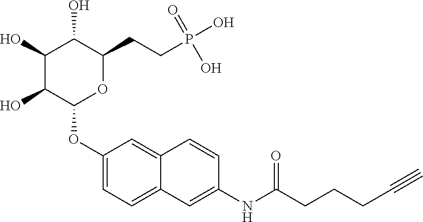 [SMILES: C#CCCCC(=O)Nc1ccc2cc(O[C@H]3O[C@H](CCP(=O)(O)O)[C@@H](O)[C@H](O)[C@@H]3O)ccc2c1] |
| X10 | 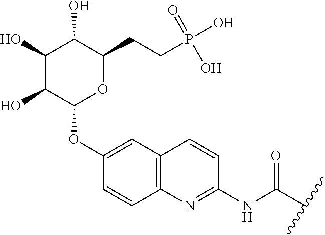 [SMILES: *C(=O)Nc1ccc2cc(O[C@H]3O[C@H](CCP(=O)(O)O)[C@@H](O)[C@H](O)[C@@H]3O)ccc2n1] | 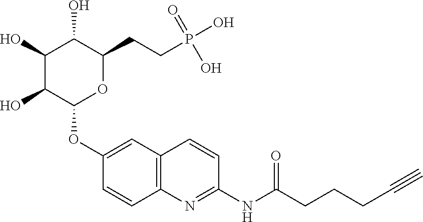 [SMILES: C#CCCCC(=O)Nc1ccc2cc(O[C@H]3O[C@H](CCP(=O)(O)O)[C@@H](O)[C@H](O)[C@@H]3O)ccc2n1] |
| X11 | 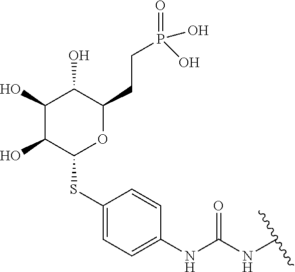 [SMILES: *NC(=O)Nc1ccc(S[C@H]2O[C@H](CCP(=O)(O)O)[C@@H](O)[C@H](O)[C@@H]2O)cc1] | 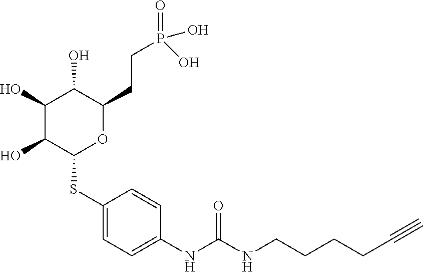 [SMILES: C#CCCCCNC(=O)Nc1ccc(S[C@H]2O[C@H](CCP(=O)(O)O)[C@@H](O)[C@H](O)[C@@H]2O)cc1] |
| X11* | 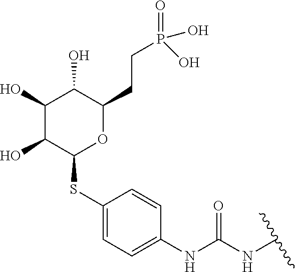 [SMILES: *NC(=O)Nc1ccc(S[C@@H]2O[C@H](CCP(=O)(O)O)[C@@H](O)[C@H](O)[C@@H]2O)cc1] | 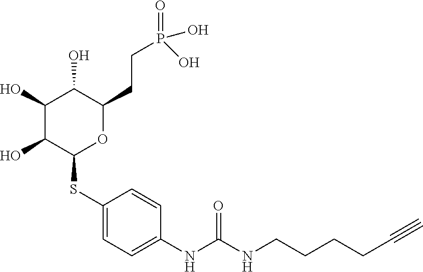 [SMILES: C#CCCCCNC(=O)Nc1ccc(S[C@@H]2O[C@H](CCP(=O)(O)O)[C@@H](O)[C@H](O)[C@@H]2O)cc1] |
| X12 | 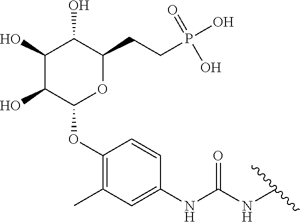 [SMILES: *NC(=O)Nc1ccc(O[C@H]2O[C@H](CCP(=O)(O)O)[C@@H](O)[C@H](O)[C@@H]2O)c(C)c1] | 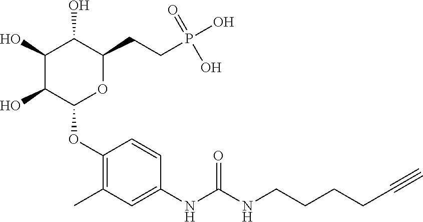 [SMILES: C#CCCCCNC(=O)Nc1ccc(O[C@H]2O[C@H](CCP(=O)(O)O)[C@@H](O)[C@H](O)[C@@H]2O)c(C)c1] |
| X13 | 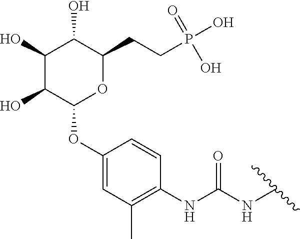 [SMILES: *NC(=O)Nc1ccc(O[C@H]2O[C@H](CCP(=O)(O)O)[C@@H](O)[C@H](O)[C@@H]2O)cc1C] | 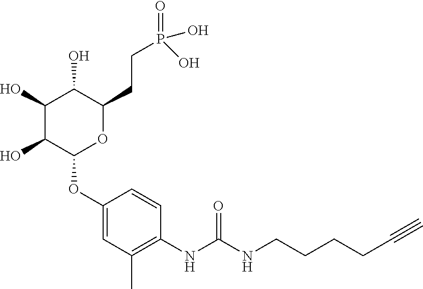 [SMILES: C#CCCCCNC(=O)Nc1ccc(O[C@H]2O[C@H](CCP(=O)(O)O)[C@@H](O)[C@H](O)[C@@H]2O)cc1C] |
| X14 | 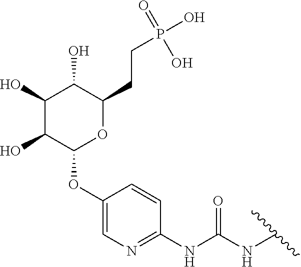 [SMILES: *NC(=O)Nc1ccc(O[C@H]2O[C@H](CCP(=O)(O)O)[C@@H](O)[C@H](O)[C@@H]2O)cn1] | 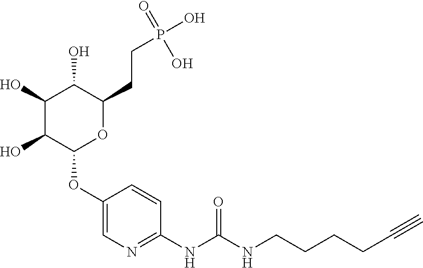 [SMILES: C#CCCCCNC(=O)Nc1ccc(O[C@H]2O[C@H](CCP(=O)(O)O)[C@@H](O)[C@H](O)[C@@H]2O)cn1] |
| X15 | 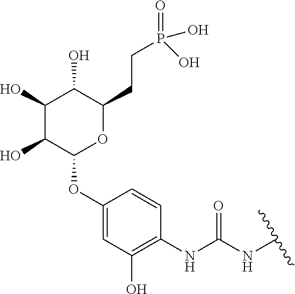 [SMILES: *NC(=O)Nc1ccc(O[C@H]2O[C@H](CCP(=O)(O)O)[C@@H](O)[C@H](O)[C@@H]2O)cc1O] | 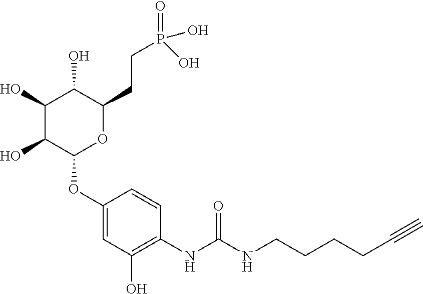 [SMILES: C#CCCCCNC(=O)Nc1ccc(O[C@H]2O[C@H](CCP(=O)(O)O)[C@@H](O)[C@H](O)[C@@H]2O)cc1O] |
| X16 | 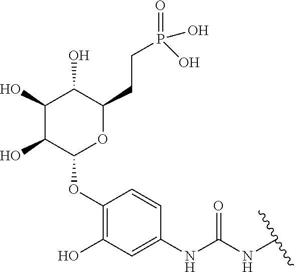 [SMILES: *NC(=O)Nc1ccc(O[C@H]2O[C@H](CCP(=O)(O)O)[C@@H](O)[C@H](O)[C@@H]2O)c(O)c1] | 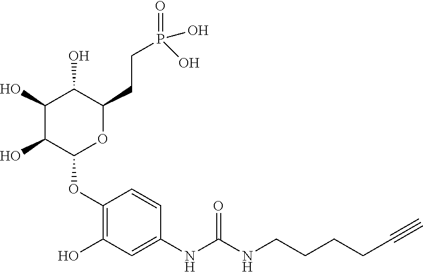 [SMILES: C#CCCCCNC(=O)Nc1ccc(O[C@H]2O[C@H](CCP(=O)(O)O)[C@@H](O)[C@H](O)[C@@H]2O)c(O)c1] |
| X17 | 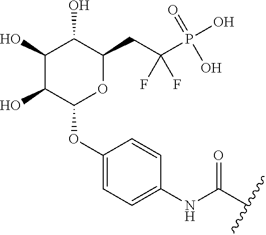 [SMILES: *C(=O)Nc1ccc(O[C@H]2O[C@H](CC(F)(F)P(=O)(O)O)[C@@H](O)[C@H](O)[C@@H]2O)cc1] | 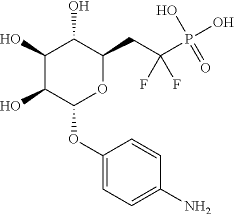 [SMILES: Nc1ccc(O[C@H]2O[C@H](CC(F)(F)P(=O)(O)O)[C@@H](O)[C@H](O)[C@@H]2O)cc1] |
| X18 | 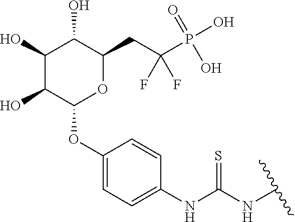 [SMILES: *NC(=S)Nc1ccc(O[C@H]2O[C@H](CC(F)(F)P(=O)(O)O)[C@@H](O)[C@H](O)[C@@H]2O)cc1] | 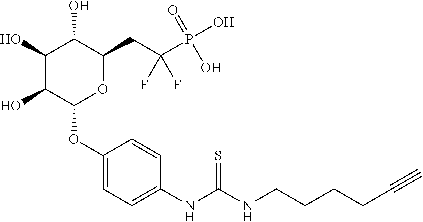 [SMILES: C#CCCCCNC(=S)Nc1ccc(O[C@H]2O[C@H](CC(F)(F)P(=O)(O)O)[C@@H](O)[C@H](O)[C@@H]2O)cc1] |
| X19 | 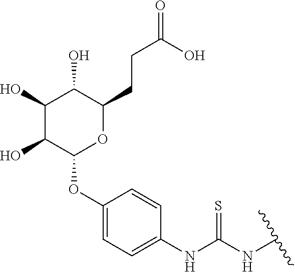 [SMILES: *NC(=S)Nc1ccc(O[C@H]2O[C@H](CCC(=O)O)[C@@H](O)[C@H](O)[C@@H]2O)cc1] | 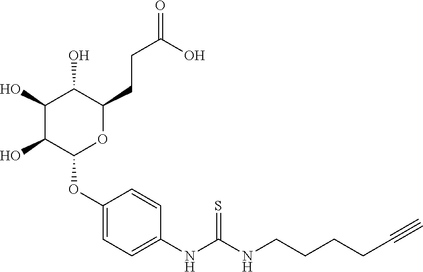 [SMILES: C#CCCCCNC(=S)Nc1ccc(O[C@H]2O[C@H](CCC(=O)O)[C@@H](O)[C@H](O)[C@@H]2O)cc1] |
| X20 | 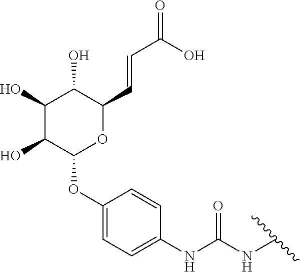 [SMILES: *NC(=O)Nc1ccc(O[C@H]2O[C@H](/C=C/C(=O)O)[C@@H](O)[C@H](O)[C@@H]2O)cc1] | 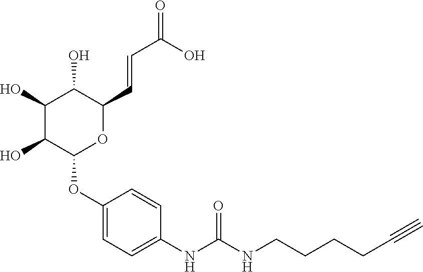 [SMILES: C#CCCCCNC(=O)Nc1ccc(O[C@H]2O[C@H](/C=C/C(=O)O)[C@@H](O)[C@H](O)[C@@H]2O)cc1] |
| X21 | 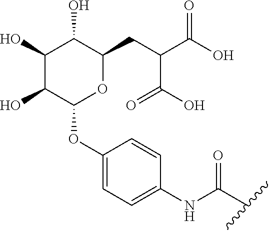 [SMILES: *C(=O)Nc1ccc(O[C@H]2O[C@H](CC(C(=O)O)C(=O)O)[C@@H](O)[C@H](O)[C@@H]2O)cc1] | 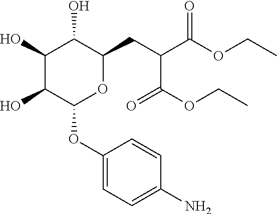 [SMILES: CCOC(=O)C(C[C@H]1O[C@H](Oc2ccc(N)cc2)[C@@H](O)[C@@H](O)[C@@H]1O)C(=O)OCC] |
| X22 | 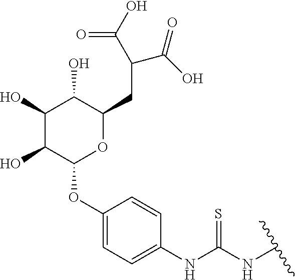 [SMILES: *NC(=S)Nc1ccc(O[C@H]2O[C@H](CC(C(=O)O)C(=O)O)[C@@H](O)[C@H](O)[C@@H]2O)cc1] | 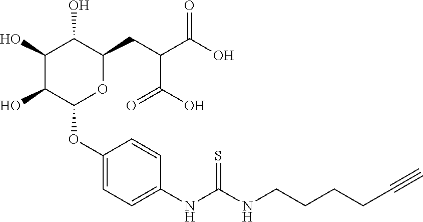 [SMILES: C#CCCCCNC(=S)Nc1ccc(O[C@H]2O[C@H](CC(C(=O)O)C(=O)O)[C@@H](O)[C@H](O)[C@@H]2O)cc1] |
| X23 | 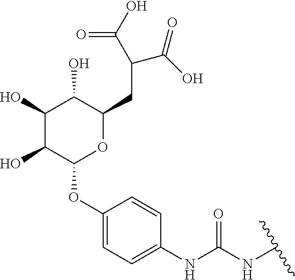 [SMILES: *NC(=O)Nc1ccc(O[C@H]2O[C@H](CC(C(=O)O)C(=O)O)[C@@H](O)[C@H](O)[C@@H]2O)cc1] | 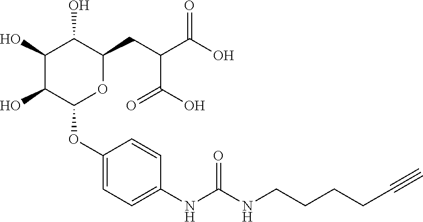 [SMILES: C#CCCCCNC(=O)Nc1ccc(O[C@H]2O[C@H](CC(C(=O)O)C(=O)O)[C@@H](O)[C@H](O)[C@@H]2O)cc1] |
| X24 | 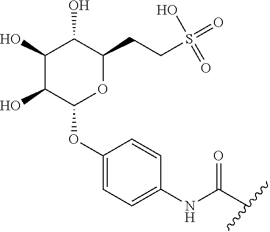 [SMILES: *C(=O)Nc1ccc(O[C@H]2O[C@H](CCS(=O)(=O)O)[C@@H](O)[C@H](O)[C@@H]2O)cc1] | 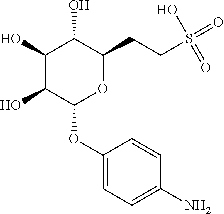 [SMILES: Nc1ccc(O[C@H]2O[C@H](CCS(=O)(=O)O)[C@@H](O)[C@H](O)[C@@H]2O)cc1] |
| X25 | 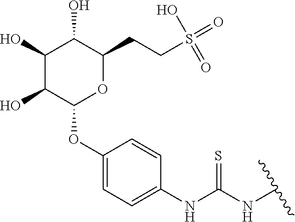 [SMILES: *NC(=S)Nc1ccc(O[C@H]2O[C@H](CCS(=O)(=O)O)[C@@H](O)[C@H](O)[C@@H]2O)cc1] | 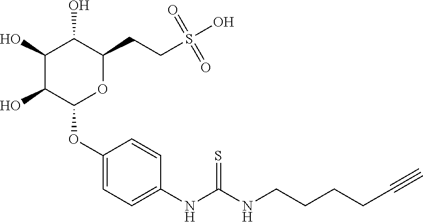 [SMILES: C#CCCCCNC(=S)Nc1ccc(O[C@H]2O[C@H](CCS(=O)(=O)O)[C@@H](O)[C@H](O)[C@@H]2O)cc1] |
| X26 | 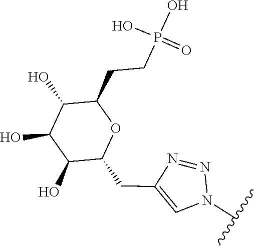 [SMILES: *n1cc(C[C@H]2O[C@H](CCP(=O)(O)O)[C@@H](O)[C@H](O)[C@@H]2O)nn1] | 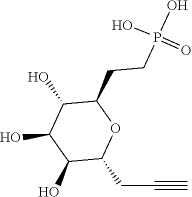 [SMILES: C#CC[C@H]1O[C@H](CCP(=O)(O)O)[C@@H](O)[C@H](O)[C@@H]1O] |
| X27 | 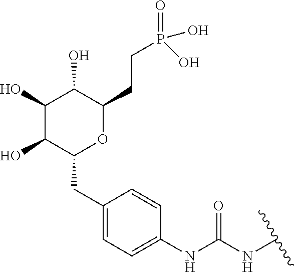 [SMILES: *NC(=O)Nc1ccc(C[C@H]2O[C@H](CCP(=O)(O)O)[C@@H](O)[C@H](O)[C@@H]2O)cc1] | 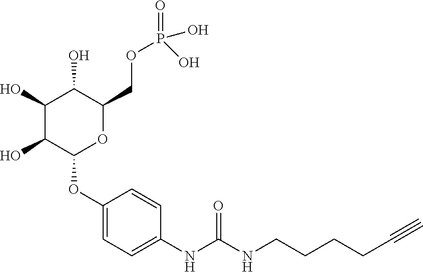 [SMILES: C#CCCCCNC(=O)Nc1ccc(O[C@H]2O[C@H](COP(=O)(O)O)[C@@H](O)[C@H](O)[C@@H]2O)cc1] |
| X28 | 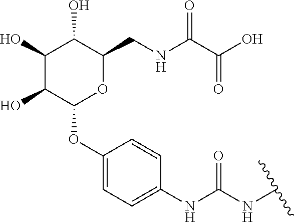 [SMILES: *NC(=O)Nc1ccc(O[C@H]2O[C@H](CNC(=O)C(=O)O)[C@@H](O)[C@H](O)[C@@H]2O)cc1] | 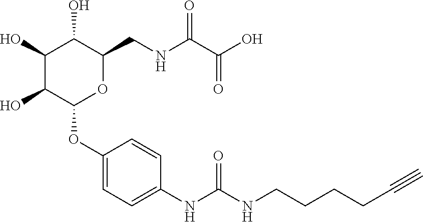 [SMILES: C#CCCCCNC(=O)Nc1ccc(O[C@H]2O[C@H](CNC(=O)C(=O)O)[C@@H](O)[C@H](O)[C@@H]2O)cc1] |
| X29 | 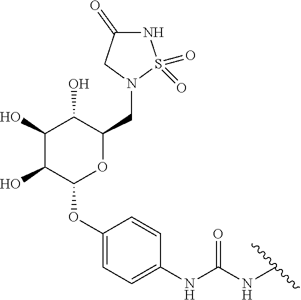 [SMILES: *NC(=O)Nc1ccc(O[C@H]2O[C@H](CN3CC(=O)NS3(=O)=O)[C@@H](O)[C@H](O)[C@@H]2O)cc1] | 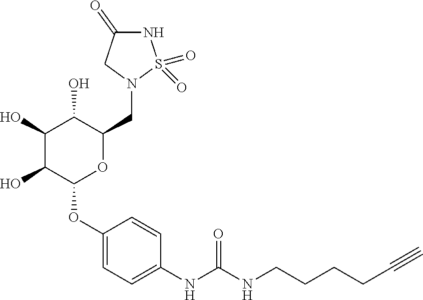 [SMILES: C#CCCCCNC(=O)Nc1ccc(O[C@H]2O[C@H](CN3CC(=O)NS3(=O)=O)[C@@H](O)[C@H](O)[C@@H]2O)cc1] |
| X30 | 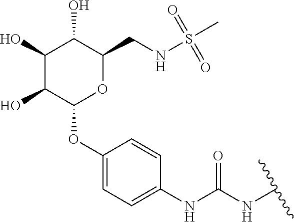 [SMILES: *NC(=O)Nc1ccc(O[C@H]2O[C@H](CNS(C)(=O)=O)[C@@H](O)[C@H](O)[C@@H]2O)cc1] | 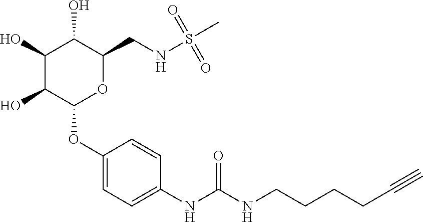 [SMILES: C#CCCCCNC(=O)Nc1ccc(O[C@H]2O[C@H](CNS(C)(=O)=O)[C@@H](O)[C@H](O)[C@@H]2O)cc1] |
| X31 | 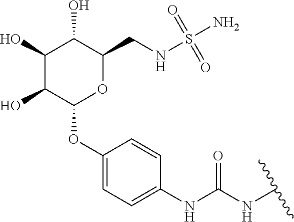 [SMILES: *NC(=O)Nc1ccc(O[C@H]2O[C@H](CNS(N)(=O)=O)[C@@H](O)[C@H](O)[C@@H]2O)cc1] | 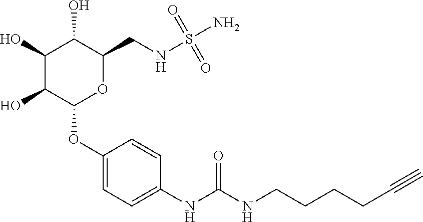 [SMILES: C#CCCCCNC(=O)Nc1ccc(O[C@H]2O[C@H](CNS(N)(=O)=O)[C@@H](O)[C@H](O)[C@@H]2O)cc1] |
| X32 | 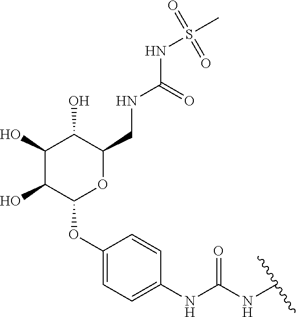 [SMILES: *NC(=O)Nc1ccc(O[C@H]2O[C@H](CNC(=O)NS(C)(=O)=O)[C@@H](O)[C@H](O)[C@@H]2O)cc1] | 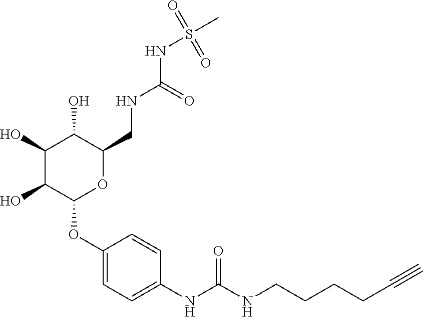 [SMILES: C#CCCCCNC(=O)Nc1ccc(O[C@H]2O[C@H](CNC(=O)NS(C)(=O)=O)[C@@H](O)[C@H](O)[C@@H]2O)cc1] |
| X33 | 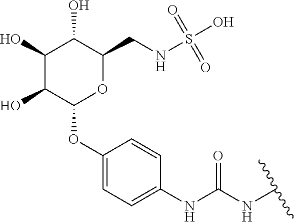 [SMILES: *NC(=O)Nc1ccc(O[C@H]2O[C@H](CNS(=O)(=O)O)[C@@H](O)[C@H](O)[C@@H]2O)cc1] | 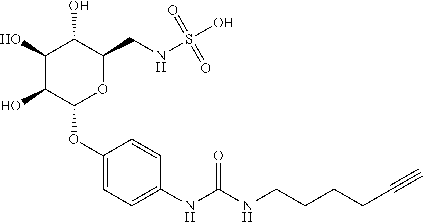 [SMILES: C#CCCCCNC(=O)Nc1ccc(O[C@H]2O[C@H](CNS(=O)(=O)O)[C@@H](O)[C@H](O)[C@@H]2O)cc1] |
| X34 | 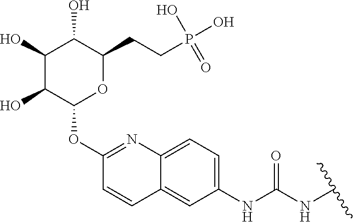 [SMILES: *NC(=O)Nc1ccc2nc(O[C@H]3O[C@H](CCP(=O)(O)O)[C@@H](O)[C@H](O)[C@@H]3O)ccc2c1] | 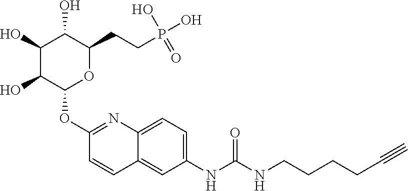 [SMILES: C#CCCCCNC(=O)Nc1ccc2nc(O[C@H]3O[C@H](CCP(=O)(O)O)[C@@H](O)[C@H](O)[C@@H]3O)ccc2c1] |
| X35 | 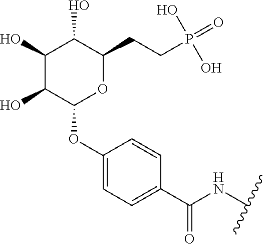 [SMILES: *NC(=O)c1ccc(O[C@H]2O[C@H](CCP(=O)(O)O)[C@@H](O)[C@H](O)[C@@H]2O)cc1] | 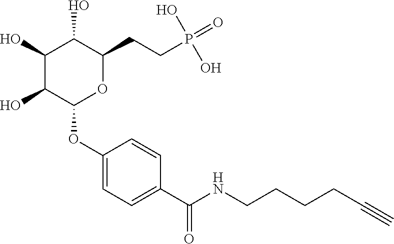 [SMILES: C#CCCCCNC(=O)c1ccc(O[C@H]2O[C@H](CCP(=O)(O)O)[C@@H](O)[C@H](O)[C@@H]2O)cc1] |
| X36 | 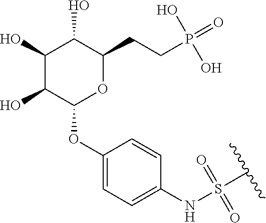 [SMILES: *S(=O)(=O)Nc1ccc(O[C@H]2O[C@H](CCP(=O)(O)O)[C@@H](O)[C@H](O)[C@@H]2O)cc1] | 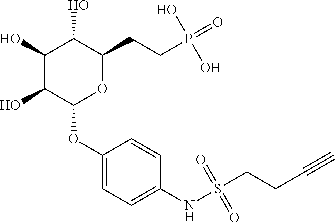 [SMILES: C#CCCS(=O)(=O)Nc1ccc(O[C@H]2O[C@H](CCP(=O)(O)O)[C@@H](O)[C@H](O)[C@@H]2O)cc1] |
| X37 | 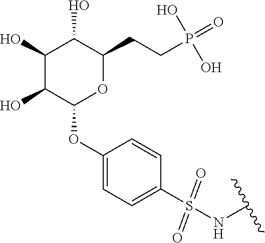 [SMILES: *NS(=O)(=O)c1ccc(O[C@H]2O[C@H](CCP(=O)(O)O)[C@@H](O)[C@H](O)[C@@H]2O)cc1] | 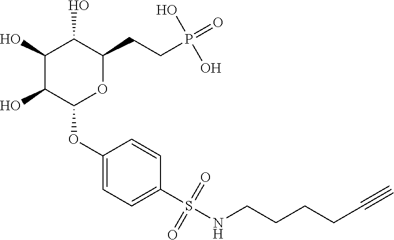 [SMILES: C#CCCCCNS(=O)(=O)c1ccc(O[C@H]2O[C@H](CCP(=O)(O)O)[C@@H](O)[C@H](O)[C@@H]2O)cc1] |
| X38 | 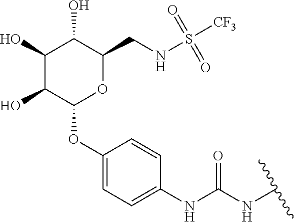 [SMILES: *NC(=O)Nc1ccc(O[C@H]2O[C@H](CNS(=O)(=O)C(F)(F)F)[C@@H](O)[C@H](O)[C@@H]2O)cc1] | 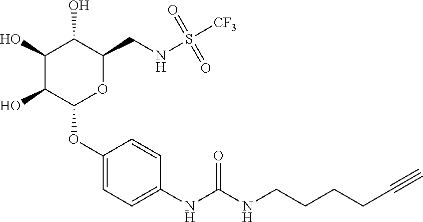 [SMILES: C#CCCCCNC(=O)Nc1ccc(O[C@H]2O[C@H](CNS(=O)(=O)C(F)(F)F)[C@@H](O)[C@H](O)[C@@H]2O)cc1] |
| X27* | 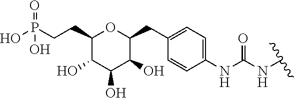 [SMILES: *NC(=O)Nc1ccc(C[C@@H]2O[C@H](CCP(=O)(O)O)[C@@H](O)[C@H](O)[C@@H]2O)cc1] | 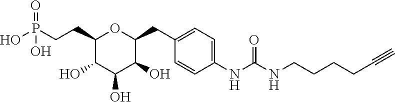 [SMILES: C#CCCCCNC(=O)Nc1ccc(C[C@@H]2O[C@H](CCP(=O)(O)O)[C@@H](O)[C@H](O)[C@@H]2O)cc1] |
| X39* | 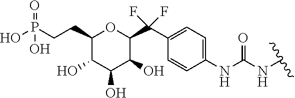 [SMILES: *NC(=O)Nc1ccc(C(F)(F)[C@@H]2O[C@H](CCP(=O)(O)O)[C@@H](O)[C@H](O)[C@@H]2O)cc1] | 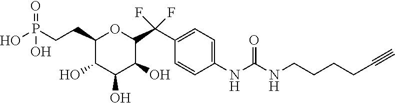 |
| X39 | 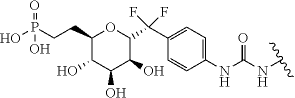 | 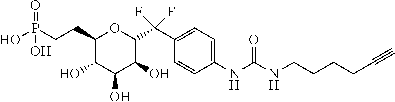 |
| X2* | 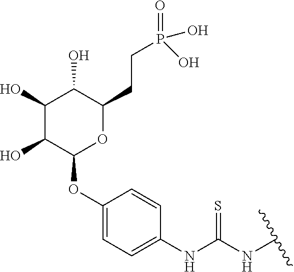 [SMILES: *NC(=S)Nc1ccc(O[C@@H]2O[C@H](CCP(=O)(O)O)[C@@H](O)[C@H](O)[C@@H]2O)cc1] | 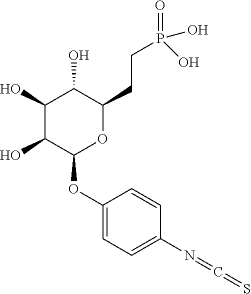 [SMILES: O=P(O)(O)CC[C@H]1O[C@@H](Oc2ccc(N=C=S)cc2)[C@@H](O)[C@@H](O)[C@@H]1O] |
| X3* | 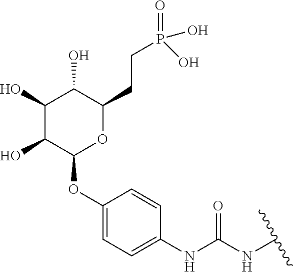 [SMILES: *NC(=O)Nc1ccc(O[C@@H]2O[C@H](CCP(=O)(O)O)[C@@H](O)[C@H](O)[C@@H]2O)cc1] | 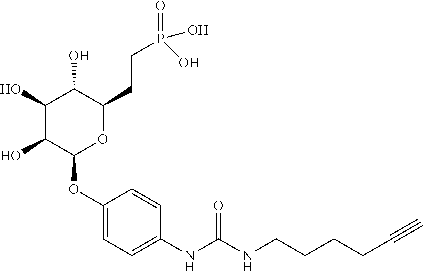 [SMILES: C#CCCCCNC(=O)Nc1ccc(O[C@@H]2O[C@H](CCP(=O)(O)O)[C@@H](O)[C@H](O)[C@@H]2O)cc1] |

Other M6PR binding moieties of interest and synthons or synthetic precursors thereof, are shown in Table 25. X101-X103 show compounds having a phosphate ester or thiophosphate ester head group. X109-X110 show exemplary compounds of formula (V). In some embodiments, such M6PR binding moieties are used in reference compounds for the assessment of compounds of formula (XII).

TABLE 25
Other Exemplary M6PR binding moieties and synthetic precursors

| Exemplary X for M6PR binding compounds | | Exemplary Synthetic precursors |
|---|---|---|
| # | Structure | Structure |
| X101 | 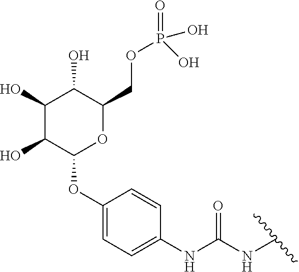 [SMILES: *NC(=O)Nc1ccc(O[C@H]2O[C@H](COP(=O)(O)O)[C@@H](O)[C@H](O)[C@@H]2O)cc1] | 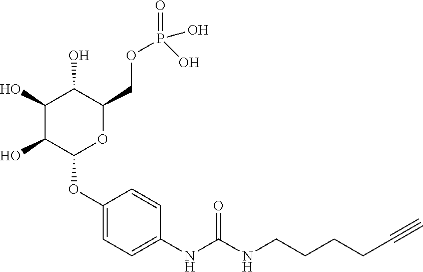 [SMILES: C#CCCCCNC(=O)Nc1ccc(O[C@H]2O[C@H](COP(=O)(O)O)[C@@H](O)[C@H](O)[C@@H]2O)cc1] |
| X102 | 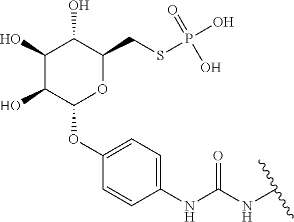 [SMILES: *NC(=O)Nc1ccc(O[C@H]2O[C@H](CSP(=O)(O)O)[C@@H](O)[C@H](O)[C@@H]2O)cc1] | 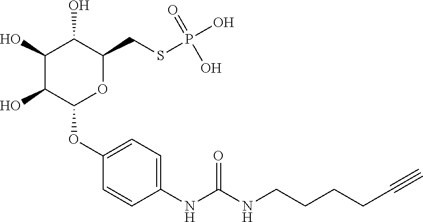 [SMILES: C#CCCCCNC(=O)Nc1ccc(O[C@H]2O[C@H](CSP(=O)(O)O)[C@@H](O)[C@H](O)[C@@H]2O)cc1] |
| X103 | 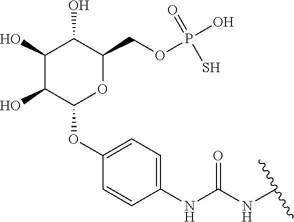 [SMILES: *NC(=O)Nc1ccc(O[C@H]2O[C@H](COP(=O)(O)S)[C@@H](O)[C@H](O)[C@@H]2O)cc1] | 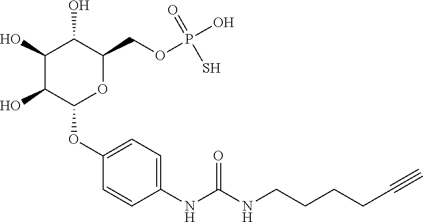 [SMILES: C#CCCCCNC(=O)Nc1ccc(O[C@H]2O[C@H](COP(=O)(O)S)[C@@H](O)[C@H](O)[C@@H]2O)cc1] |
| X104 | 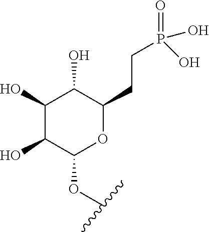 [SMILES: *O[C@H]1O[C@H](CCP(=O)(O)O)[C@@H](O)[C@H](O)[C@@H]1O] | 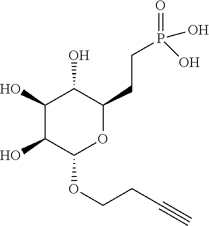 [SMILES: C#CCCO[C@H]1O[C@H](CCP(=O)(O)O)[C@@H](O)[C@H](O)[C@@H]1O] |
| X105 | 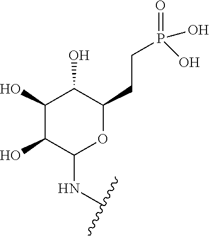 [SMILES: *NC1O[C@H](CCP(=O)(O)O)[C@@H](O)[C@H](O)[C@@H]1O] | 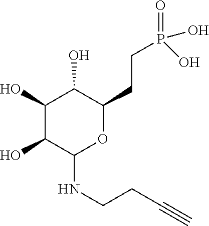 [SMILES: C#CCCNC1O[C@H](CCP(=O)(O)O)[C@@H](O)[C@H](O)[C@@H]1O] |
| X106 | 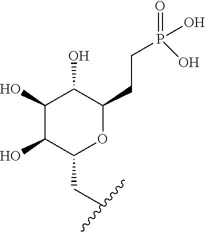 [SMILES: *C[C@H]1O[C@H](CCP(=O)(O)O)[C@@H](O)[C@H](O)[C@@H]1O] | 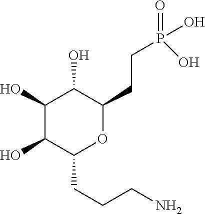 [SMILES: NCCC[C@H]1O[C@H](CCP(=O)(O)O)[C@@H](O)[C@H](O)[C@@H]1O] |
| X107 | 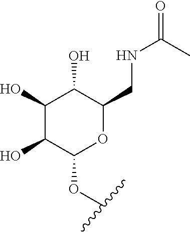 [SMILES: *O[C@H]1O[C@H](CNC(C)=O)[C@@H](O)[C@H](O)[C@@H]1O] | 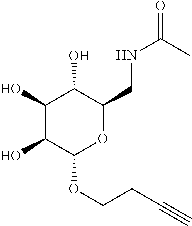 [SMILES: C#CCCO[C@H]1O[C@H](CNC(C)=O)[C@@H](O)[C@H](O)[C@@H]1O] |
| X108 | 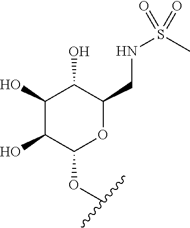 [SMILES: *O[C@H]1O[C@H](CNS(C)(=O)=O)[C@@H](O)[C@H](O)[C@@H]1O] | 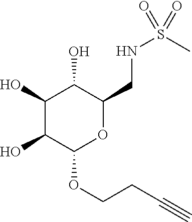 [SMILES: C#CCCO[C@H]1O[C@H](CNS(C)(=O)=O)[C@@H](O)[C@H](O)[C@@H]1O] |
| X109 | 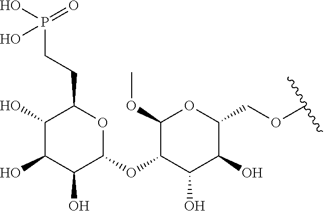 [SMILES: *OC[C@H]1O[C@H](OC)[C@@H](O[C@H]2O[C@H](CCP(=O)(O)O)[C@@H](O)[C@H](O)[C@@H]2O)[C@@H](O)[C@@H]1O] | 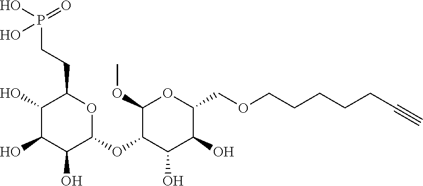 [SMILES: C#CCCCCCOC[C@H]1O[C@H](OC)[C@@H](O[C@H]2O[C@H](CCP(=O)(O)O)[C@@H](O)[C@H](O)[C@@H]2O)[C@@H](O)[C@@H]1O] |
| X110 | 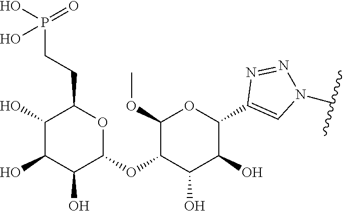 [SMILES: *n1cc([C@H]2O[C@H](OC)[C@@H](O[C@H]3O[C@H](CCP(=O)(O)O)[C@@H](O)[C@H](O)[C@@H]3O)[C@@H](O)[C@@H]2O)nn1] | 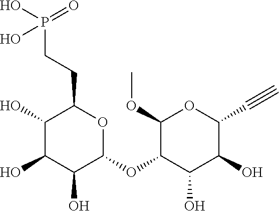 [SMILES: C#C[C@H]1O[C@H](OC)[C@@H](O[C@H]2O[C@H](CCP(=O)(O)O)[C@@H](O)[C@H](O)[C@@H]2O)[C@@H](O)[C@@H]1O] |

### Conjugates

The compounds of this disclosure can be referred to as a conjugate, e.g., when the moiety of interest (Y) is an antibody or antibody fragment (e.g., as described herein). In one embodiment, the conjugate comprises a ligand moiety conjugated via a linker to a target-binding moiety wherein the ligand moiety binds a lysosomal targeting molecule extracellularly; the target-binding moiety binds a target molecule extracellularly; the target-binding moiety dissociates from the target molecular intraendosomally and the conjugate is externalized from a cell. Such conjugates can be prepared by conjugation of a chemoselective ligation group of any one of the compounds described herein with a compatible reactive group of a molecule Y. The compatible group of the molecule Y can be introduced by modification prior to conjugation, or can be a group present in the molecule. Alternatively, such conjugates can be prepared de novo, e.g., via modification of a Y molecule of interest starting material to introduce a linker, e.g., to which a ligand or lysosomal targeting molecule binding moiety (X) can be attached.

In some embodiments, the moiety of interest to which the lysosomal targeting molecule binding moiety is linked is a biomolecule. In some embodiments, the moiety of interest is a biomolecule. In some embodiments, Y is a biomolecule that specifically binds to a target molecule, such as a target protein. In some embodiments, the biomolecule is an antibody, or antibody fragment.

In some embodiments, the moiety of interest is a molecule that specifically binds to a target of interest, i.e., a target-binding moiety. In such cases, the conjugates of this disclosure can provide for cellular uptake of the target after it non-covalently binds to the conjugate, and/or degradation. The inventors have demonstrated that conjugates of this disclosure having a particular configuration of lysosomal targeting molecule binding moiety of a desired affinity, with a linker of desired valency and length that can specifically bind with high affinity to both the lysosomal targeting molecule and the target simultaneously. The conjugates of this disclosure can thus provide for sequestering of a target protein in the cell's lysosome and degrading of the target protein.

The compounds of this disclosure can, in some cases, be referred to as a conjugate, e.g., when the moiety of interest (Y) is a molecule such as a biomolecule, where the conjugate can be derived from a conjugation or coupling reaction between a chemoselective ligation group and a compatible group on the biomolecule. In some embodiments, the biomolecule is conjugated via a naturally occurring group of the biomolecule. In some embodiments, the biomolecule is conjugated via a compatible functional group that is introduced into the biomolecule prior to chemoselective conjugation. In such cases, the linking moiety between X and Y incorporates the residual group (e.g., Z) that is the product of the chemoselective ligation chemistry.

Aspects of this disclosure include compounds of formula (I) where the moiety of interest Y is a moiety that specifically binds to a target molecule, such as a target protein. The target protein can be the target protein is a membrane bound protein or an extracellular protein. In some embodiments of the compounds of this disclosure, Y is a biomolecule that specifically binds to a target protein. In some embodiments, the conjugate includes a moiety of interest Y that specifically binds a target protein, and can find use in methods of cell uptake or internalization of the target protein via binding to the cell surface receptor, and eventual degradation of the target protein.

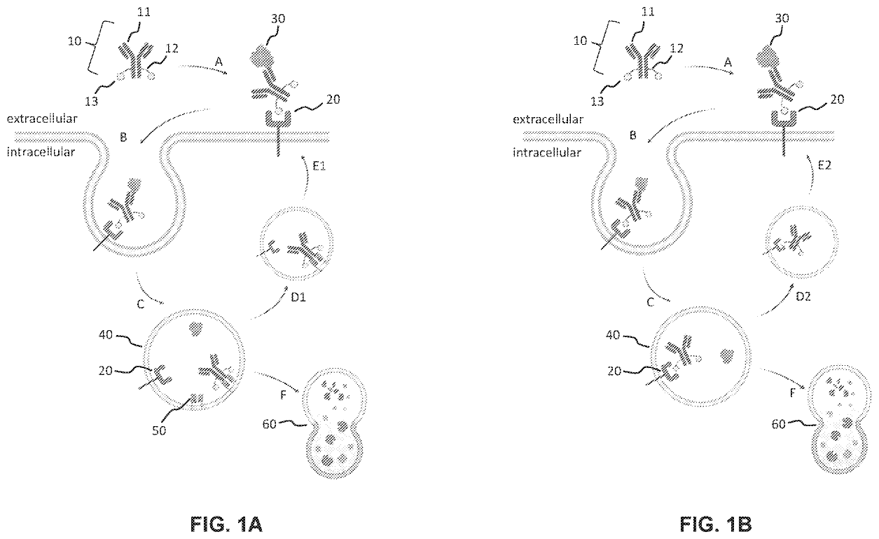

In one embodiment, the conjugate can facilitate degradation of a target and can repeatedly “cycle” into and out of a cell. Either of two cycling mechanisms are hypothesized to occur, and in some cases both mechanisms may occur with respect to a given conjugate. Referring to FIG. 1A, conjugate 10 includes target-binding moiety 11, linker 12, and ligand moiety 13. In step A, conjugate 10 binds a lysosomal targeting molecule 20 extracellularly and also binds a target 30 extracellularly. Such binding may occur in any temporal order. Target 30 is depicted as a soluble target but, as described herein may be a transmembrane target or other target. The resulting ternary complex is internalized into an intracellular vesicle, step B, forming an endosome 40 in step C. Within endosome 40, a first cycling mechanism occurs: (i) the conjugate dissociates from the lysosomal targeting molecule; (ii) the conjugate dissociates from the target; and (iii) the conjugate binds to FcRn 50, with events (i), (ii) and (iii) occurring in any temporal order. As shown in steps D1, E1, and F, the conjugate and the target have separate fates: in step D1, the conjugate remains in the endosome and in step E1 is externalized from the cell, whereas in step F, the target is degraded in lysosome 60.

FIG. 1B shows some of the same components, features and steps as shown in FIG. 1A. However, in this second cycling mechanism, within endosome 40, the conjugate remains bound to the lysosomal targeting molecule 20 and is thereby externalized from the cell in steps D2 and E2. The target, as with the previous mechanism, is degraded in lysosome 60 in step F.

Utilizing these mechanisms, conjugates of the present disclosure are “cycled” in and out of a cell (internalized and externalized) and facilitate lysosomal degradation of a target. Such cycling enables duration of activity on the order of hours to days, and the ability for a single conjugate to facilitate lysosomal degradation of multiple targets. In other words, the conjugates described herein may be used to degrade super-stoichiometric ratios of target by repeatedly binding a target, internalizing with the target to facilitating its lysosomal degradation, and cycling back to the cell membrane to bind an additional target.

In some embodiments, the conjugate is internalized into a cell via a lysosomal targeting molecule, for example after the conjugate binds to the lysosomal trafficking receptor. Receptor-mediated internalization is described, for example, in G. Ahn et al., Nat. Chem. Biol. 2021, 17(9) 937-46 and references cited therein. As described elsewhere herein, in various embodiments, the lysosomal targeting molecule is ASGPR. In other embodiments, the lysosomal targeting molecule is M6PR. In other embodiments, the lysosomal targeting molecule is LDLR or CD63.

In some embodiments, ligand moiety, X, remains bound to the lysosomal targeting molecule intraendosomally. In some embodiments, the conjugate is externalized from the cell via the lysosomal targeting molecule. In some embodiments, the ligand moiety has an equal binding affinity for the lysosomal targeting molecule extracellularly and intraendosomally. In some embodiments, the ligand moiety, X, has an equal binding affinity for the lysosomal targeting molecule at an extracellular pH and at an intraendosomal pH. In some embodiments, the ligand moiety, X, has an equal binding affinity for the lysosomal targeting molecule at an extracellular Ca²⁺ concentration and at an intraendosomal Ca²⁺ concentration.

Externalization of the conjugate may also be mediated by FcRn. In some embodiments, the conjugate is externalized from the cell via FcRn, for example after target-binding moiety, Y, binds FcRn. In some embodiments, the ligand moiety, X, binds FcRn intraendosomally. In some embodiments, the conjugate dissociates from the lysosomal targeting molecule intraendosomally. In some embodiments, the ligand moiety, X, has a higher binding affinity for the lysosomal targeting molecule extracellularly than intraendosomally. In some embodiments, the ligand moiety, X, has a higher binding affinity for the lysosomal targeting molecule at an extracellular pH than at an intraendosomal pH. In some embodiments, the ligand moiety, X, has a higher binding affinity for the lysosomal targeting molecule at an extracellular Ca²⁺ concentration than at an intraendosomal Ca²⁺ concentration. In some embodiments, the ratio of binding affinity of ligand moiety, X, to the lysosomal targeting molecule intraendosomally:extracellularly is between 1:2 and 2:1. In some embodiments, the ratio of binding affinity of ligand moiety, X, to the lysosomal targeting molecule intraendosomally:extracellularly is between 1:5 and 5:1. In some embodiments, the ratio of binding affinity of ligand moiety, X, to the lysosomal targeting molecule intraendosomally:extracellularly is between 1:10 and 10:1.

In some embodiments, the target-binding moiety, Y, has a higher binding affinity for FcRn intraendosomally than extracellularly. In some embodiments, the ratio of binding affinity of target-binding moiety, Y, to FcRn intraendosomally:extracellularly is between 1:100 and 1:10. In some embodiments, the target-binding moiety, Y, has a higher binding affinity for FcRn at an intraendosomal pH than at an extracellular pH. In some embodiments, Y, has enhanced binding to FcRn relative to wild-type at an endosomal pH. In some embodiments, the target-binding moiety, Y, has approximately equal binding affinity to FcRn extracellularly as wild-type IgG does extracellularly. In some embodiments, the target-binding moiety, Y, has, at pH 7.4, approximately equal binding affinity to FcRn as wild-type IgG.

In some embodiments, Y is a mutant form of an antibody, Y has the YTE mutation.

In one method is provided a method of degrading a target molecule in a subject in need thereof, comprising administering an effective amount of a conjugate that comprises:
a means for binding a lysosomal targeting molecule extracellularly;
a means for binding a target molecule extracellularly;
a means for dissociating from the target molecule intraendosomally; and
wherein the conjugate is externalized from a cell.

In one embodiment, the means for binding a lysosomal targeting molecule, remains bound to the lysosomal targeting molecule intraendosomally. In one embodiment, the means for binding a target molecule also binds FcRn intraendosomally. In one embodiment, the conjugate dissociates from the lysosomal targeting molecule intraendosomally.

In some embodiments, one Y biomolecule is conjugated to a single moiety (X) that specifically binds to the cell surface receptor (e.g., ASGPR, M6PR, FR) via a linker L. In some embodiments, one Y biomolecule is conjugated to one (Xn-L)- group, wherein when n=1 the (Xn-L)- group is referred to as monovalent, and when n>1 the (Xn-L)- group is referred to as multivalent (e.g., bivalent, trivalent, tetravalent, etc.). It is understood that in some embodiments of formula (I), where Y is a biomolecule, Y can be conjugated to two or more (Xn-L)- groups, wherein each (Xn-L)- group may itself be monovalent or multivalent (e.g., bivalent, trivalent, tetravalent, etc.). In such cases, the ratio of linked (Xn-L)- groups to biomolecule can be referred to as 2 or more.

In some embodiments, the conjugation of the one or more (Xn-L)- groups (e.g., m is 1, 2, or 3) to a Y results in the generation of a residual moiety resulting from the covalent linkage of a chemoselective ligation group to a compatible group of Y. For example, conjugates of this disclosure can be prepared using the building blocks described herein as exemplified in Scheme 1. In Scheme 1, conjugates of formula (I′):

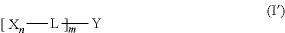

is represented by formula (II′):

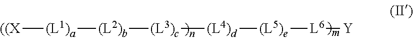

wherein:
n is 1 to 3;
m is 1 to 20;
a, b, c, d, and e are each independently 1, 2, 3, 4, or 5;
each L¹ to L⁶ is independently a linking moiety which together provide a linear or branched linker between Z¹ and Y;
X and Y are as defined herein.

Scheme I is intended to be exemplary and in no way is intended to limit the scope of the disclosure. However, as can be appreciated by one of skill in the art, the compounds of this disclosure have various L moieties which may be constructed by coupling X to one or more first portions of the linker L (e.g., an -L¹- moiety) via Z¹ to provide exemplary target binding moiety, or X, building blocks. In Scheme 1, R^{M1} and R^{M2} are each independently reactive functional groups for coupling reactions (e.g., alkyne, —N₃, —C(O)OH, —NH₂, etc.); and Y¹ is a chemoselective ligation group capable of conjugating to an amino acid residue(s) of Y.

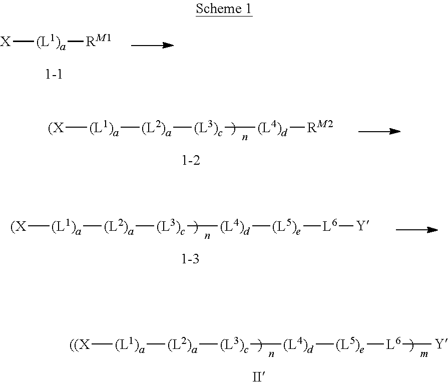

Methods for the steps and exemplary reagents and starting materials (i.e., compounds of Formula 1-1, 1-2, 1-3) are described throughout or can be derived from the art.

In some embodiments, Y is an antibody or antibody fragment that specifically binds the target protein and the compound is a conjugate of formula (III):

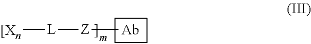

wherein:
n is 1 to 20;
m is an average loading of 1 to 80;
each X is a ligand moiety that binds to a lysosomal targeting molecule; each L is a linker;
each Z is a residual moiety resulting from the covalent linkage of a chemoselective ligation group to a compatible group of Ab; and
Ab is the antibody or antibody fragment that specifically binds the target protein.

In certain embodiments of the conjugate of formula (III), L is a linker of formula (II) (e.g., as described herein).

In certain embodiments of the conjugate of formula (III), n is 1 to 6. In certain cases, n is 1, such that the antibody is conjugated to a monovalent ligand and the linker is of the formula (IIa) (e.g., as described herein). In certain cases, n is at least 2, such that the antibody is conjugated to a multivalent ligand. In certain cases, n is 2. In certain cases n is 3.

In certain embodiments of the conjugate of formula (III), Z is a residual moiety resulting from the covalent linkage of a chemoselective ligation moiety (e.g., Table 10 and Table 11).

In certain embodiments of the conjugate of formula (III), Z is a residual moiety resulting from the covalent linkage of a thiol reactive chemoselective ligation group to one or more cysteine residue(s) of Ab.

In certain other embodiments of the conjugate of formula (III), Z is a residual moiety resulting from the covalent linkage of an amine-reactive chemoselective ligation group to one or more lysine residue(s) of Ab.

In certain embodiments, the conjugates with their linker structures described herein have weaker binding affinity to cell surface receptors. Without being bound to any particular mechanism or theory, such weaker binding affinity may be corrected to longer half-life of the conjugates, and may be useful for tuning (e.g., modifying) the pharmacokinetic properties of the conjugates described herein. In certain embodiments, such weaker binding conjugates still have sufficiently robust uptake.

Conjugates of a polypeptide, e.g., an antibody (Ab) and compound (Xn-L-Y) may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In certain embodiments of the conjugates described herein, L is bonded through an amide bond to a lysine residue of the polypeptide. In certain embodiments of the conjugates described herein, L is bonded through a thioether bond to a cysteine residue of the polypeptide. In certain embodiments of the conjugates described herein, L is bonded through an amide bond to a lysine residue of Ab. In certain embodiments of the conjugates described herein, L is bonded through a thioether bond to a cysteine residue of Ab. In certain embodiments of the conjugates described herein, L is bonded through two thioether bonds to two cysteine residues of Ab, wherein the two cysteine residues are from an opened cysteine-cysteine disulfide bond in Ab. In certain embodiments, the opened cysteine-cysteine disulfide bond is an interchain disulfide bond.

In certain embodiments of the conjugates described herein, when L is bonded through an amide bond to a lysine residue of a polypeptide (e.g., an antibody), m is an integer from 1 to 80. In certain embodiments of the conjugates described herein, when L is bonded through a thioether bond to a cysteine residue of P, m is an integer from 1 to 8.

In certain embodiments, conjugation to the polypeptide, or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety. All such methodologies are contemplated for use in connection with making the conjugates described herein.

Loading of the compounds of formula (I) to the polypeptides (e.g., antibodies) described herein is represented by “m” in formula (III), and is the average number of units of “Xn-L-” or “Xn-” per conjugate molecule. As used herein, the term “DAR” refers to the average value of “m” or the loading of the conjugate. The number of “X” moieties (e.g., folate moieties) per each unit of “Xn-L-” or “Xn-” is represented by “n” in formula (III). As used herein, the term “valency” or “valencies” refers to the number of “X” moieties per unit (“n”). It will be understood that loading, or DAR, is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for the conjugates described herein, the total number of “X” moieties per conjugate molecule will be n x m. As used herein, the term “total valency” or “total valencies” refers to the total number of “X” moieties per conjugate molecule (n x m; total valency).

DAR (loading) may range from 1 to 80 units per conjugate. The conjugates provided herein may include collections of polypeptides, antibodies or antigen binding fragments conjugated with a range of units, e.g., from 1 to 80. The average number of units per polypeptide or antibody in preparations of the conjugate from conjugation reactions may be characterized by conventional means such as mass spectroscopy. The quantitative distribution of DAR (loading) in terms of m may also be determined. In some instances, separation, purification, and characterization of homogeneous conjugate where m is a certain value may be achieved by means such as electrophoresis.

In certain embodiments, the DAR for a conjugate provided herein ranges from 1 to 80. In certain embodiments, the DAR for a conjugate provided herein ranges from 1 to 70. In certain embodiments, the DAR for a conjugate provided herein ranges from 1 to 60. In certain embodiments, the DAR for a conjugate provided herein ranges from 1 to 50. In certain embodiments, the DAR for a conjugate provided herein ranges from 1 to 40. In certain embodiments, the DAR for a conjugate provided herein ranges from 1 to 35. In certain embodiments, the DAR for a conjugate provided herein ranges from 1 to 30. In certain embodiments, the DAR for a conjugate provided herein ranges from 1 to 25. In certain embodiments, the DAR for a conjugate provided herein ranges from 1 to 20. In certain embodiments, the DAR for a conjugate provided herein ranges from 1 to 18. In certain embodiments, the DAR for a conjugate provided herein ranges from 1 to 15. In certain embodiments, the DAR for a conjugate provided herein ranges from 1 to 12. In certain embodiments, the DAR for a conjugate provided herein ranges from 1 to 10. In certain embodiments, the DAR for a conjugate provided herein ranges from 1 to 9. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4. In certain embodiments, the DAR for a conjugate provided herein ranges from 1 to 3. In certain embodiments, the DAR for a conjugate provided herein ranges from 2 to 12. In certain embodiments, the DAR for a conjugate provided herein ranges from 2 to 10. In certain embodiments, the DAR for a conjugate provided herein ranges from 2 to 9. In certain embodiments, the DAR for a conjugate provided herein ranges from 2 to 8. In certain embodiments, the DAR for a conjugate provided herein ranges from 2 to 7. In certain embodiments, the DAR for a conjugate provided herein ranges from 2 to 6. In certain embodiments, the DAR for a conjugate provided herein ranges from 2 to 5. In certain embodiments, the DAR for a conjugate provided herein ranges from 2 to 4. In certain embodiments, the DAR for a conjugate provided herein ranges from 3 to 12. In certain embodiments, the DAR for a conjugate provided herein ranges from 3 to 10. In certain embodiments, the DAR for a conjugate provided herein ranges from 3 to 9. In certain embodiments, the DAR for a conjugate provided herein ranges from 3 to 8. In certain embodiments, the DAR for a conjugate provided herein ranges from 3 to 7. In certain embodiments, the DAR for a conjugate provided herein ranges from 3 to 6. In certain embodiments, the DAR for a conjugate provided herein ranges from 3 to 5. In certain embodiments, the DAR for a conjugate provided herein ranges from 3 to 4.

In certain embodiments, the DAR for a conjugate provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9. In some embodiments, the DAR for a conjugate provided herein is about 2.2.

In some embodiments, the DAR for a conjugate provided herein ranges from 2 to 20, 2 to 19, 2 to 18, 2 to 17, 2 to 16, 2 to 15, 2 to 14, or 2 to 13. In some embodiments, the DAR for a conjugate provided herein ranges from 3 to 20, 3 to 19, 3 to 18, 3 to 17, 3 to 16, 3 to 15, 3 to 14, or 3 to 13. In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8. In some embodiments, the DAR for a conjugate provided herein is about 9. In some embodiments, the DAR for a conjugate provided herein is about 10. In some embodiments, the DAR for a conjugate provided herein is about 11. In some embodiments, the DAR for a conjugate provided herein is about 12. In some embodiments, the DAR for a conjugate provided herein is about 13. In some embodiments, the DAR for a conjugate provided herein is about 14. In some embodiments, the DAR for a conjugate provided herein is about 15. In some embodiments, the DAR for a conjugate provided herein is about 16. In some embodiments, the DAR for a conjugate provided herein is about 17. In some embodiments, the DAR for a conjugate provided herein is about 18. In some embodiments, the DAR for a conjugate provided herein is about 19. In some embodiments, the DAR for a conjugate provided herein is about 20.

In some embodiments, the DAR for a conjugate provided herein is about 25. In some embodiments, the DAR for a conjugate provided herein is about 30. In some embodiments, the DAR for a conjugate provided herein is about 35. In some embodiments, the DAR for a conjugate provided herein is about 40. In some embodiments, the DAR for a conjugate provided herein is about 50. In some embodiments, the DAR for a conjugate provided herein is about 60. In some embodiments, the DAR for a conjugate provided herein is about 70. In some embodiments, the DAR for a conjugate provided herein is about 80.

In certain embodiments, fewer than the theoretical maximum of units are conjugated to the polypeptide, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

In certain embodiments of the conjugate of formula (III) m is 1 to 20, such as 2 to 10, 2 to 8, or 2 to 6. In certain cases, m is 10 or less. In certain cases, m is 2 to 8. In certain cases, m is 2 to 6. In certain cases, m is an average loading of about 4.

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### Antibodies

In some embodiments, the target-binding moiety, Y, is an antibody or antibody fragment that specifically binds to a target molecule, such as a target protein.

The ligand moiety can be site-specifically covalently linked to the antibody or antibody fragment, via an optional linker. The ligand moiety can be covalently linked to the antibody or antibody fragment via a site-specific cysteine modification on the antibody or antibody fragment (e.g., L443C) and a thiol-reactive chemoselective ligation group. Lysosomal molecule binding moiety can be covalently linked to the antibody or antibody fragment via one or more lysine residues of the antibody or antibody fragment and an amine-reactive chemoselective ligation group.

In some embodiments wherein the target-binding moiety is an antibody, the ligand moiety is conjugated via a linker to the antibody using thiol-reactive conjugation at L443C. In some embodiments, the target-binding moiety is a mutant form of omalizumab and the linker is conjugated at L443C. In some embodiments, the target-binding moiety is a mutant form of ligelizumab and the linker is conjugated at L443C. Other sites that support thiol-reactive conjugation include Heavy Chain A118C, A140C, K392C, K290C, S293C, and Light Chain K183C, V205C, and K149C. Other sites suitable for conjugation may be used, as reported in, e.g., R. Ohri et al., Bioconjugate Chem. 2018, 19, 473-85.

In some embodiments, the conjugate of this disclosure includes an antibody (Ab), that is, the target-binding moiety, Y, is an antibody (Ab). In some embodiments, Ab is a monoclonal antibody. In some embodiments, Ab is a human antibody. In some embodiments, Ab is a humanized antibody. In some embodiments, Ab is a chimeric antibody. In some embodiments, Ab is a full-length antibody that includes two heavy chains and two light chains. In some embodiments, Ab is an IgG antibody, e.g., is an IgG1, IgG2, IgG3 or IgG4 antibody. In some embodiments, Ab is a single chain antibody. In some embodiments, the target-binding moiety is an antigen-binding fragment of an antibody, e.g., a Fab fragment.

In some embodiments, the antibody or antibody fragment comprises an Fc region. In some embodiments, the antibody or antibody fragment comprises an Fc region with one or more mutations that impart increased binding affinity of the antibody or antibody fragment for FcRn.

In some embodiments, the antibody or antibody fragment comprises an Fc region with one or more mutations selected from amino acid substitutions methionine (Met) to tyrosine (Tyr), serine (Ser) to threonine (Thr), and threonine (Thr) to glutamic acid (Glu). For example, the antibody or antibody fragment can include one or more of M252Y, S254T, and T256E (commonly referred to as YTE mutation).

In some embodiments, the antibody or antibody fragment has a binding affinity for the target molecule that is pH dependent. In some embodiments, the antibody or antibody fragment has higher binding affinity for the target molecule at neutral pH compared to low pH. In some embodiments, the antibody or antibody fragment has a binding affinity for the target molecule that is calcium dependent. In some embodiments, the antibody or antibody fragment has one or more mutations that impart pH-dependent binding affinity for the target molecule. In some embodiments, the target-binding moiety, Y, is an antibody or antibody fragment that has been mutated from the wild-type with one or more histidine substitutions. In some embodiments, the one or more histidine substitutions are located in the CDR region of the antibody or antibody fragment. For example, certain antibodies or fragment thereof can include one or more mutations from the following table:

TABLE 26

| | |
|---|---|
| Omalizumab | S35H-LC, Y57H-LC |
| Ligelizumab | W33HHC/Y50H-N100bH- |
| | HC/W94H-LC |

In examples described herein, numbering for omalizumab followed a simple sequence number, that is, for Oma S35H-LC, Y57H - LC, residue 35 corresponds to Kabat number 31 and residue 57 corresponds to Kabat number 53. Numbering for ligelizumab (e.g. Lige W33H-HC/Y50H-LC/N100bH-HC/W94H-LC) followed the Kabat numbering scheme.

In some embodiments, the target-binding moiety, Y, has a higher binding affinity for the target molecule extracellularly than intraendosomally. In some embodiments, the target-binding moiety, Y, has a higher binding affinity for the target molecule at an extracellular pH than at an intraendosomal pH. In some embodiments, the target-binding moiety, Y, has a higher binding affinity for the target molecule at a pH between about 6.5 and about 7.5 than at a pH between about 6.5 and 4.5. In some embodiments, the target-binding moiety, Y, has a higher binding affinity for the target molecule at a pH between about 7.0 and about 7.5 than at a pH between about 6.5 and 4.5. In some embodiments, the target-binding moiety, Y, has a higher binding affinity for the target molecule at a pH of about 7.4 than at a pH of about 6.0. In some embodiments, the target-binding moiety, Y, has a higher binding affinity for the target molecule at an extracellular Ca²⁺ concentration than at an intraendosomal Ca²⁺ concentration. In some embodiments, the target-binding moiety, Y, has a higher binding affinity for the target molecule at an Ca²⁺ concentration between about 1-2 mM than at an Ca²⁺ concentration between about 0-1 M.

In some embodiments, the target-binding moiety, Y, has a higher K_{D} for the target molecule intraendosomally than extracellularly. In some embodiments, the target-binding moiety, Y, has a intraendosomal:extracellular K_{D} ratio for the target molecule of between 2:1 and 10,000:1. In some embodiments, the target-binding moiety, Y, has a intraendosomal:extracellular K_{D} ratio for the target molecule of between 2:1 and 1,000:1. In some embodiments, the target-binding moiety, Y, has a intraendosomal:extracellular K_{D} ratio for the target molecule of between 2:1 and 500:1. In some embodiments, the target-binding moiety, Y, has a intraendosomal:extracellular K_{D} ratio for the target molecule of between 2:1 and 100:1. In some embodiments, the target-binding moiety, Y, has a intraendosomal:extracellular K_{D} ratio for the target molecule of between 2:1 and 50:1. In some embodiments, the target-binding moiety, Y, has a intraendosomal:extracellular K_{D} ratio for the target molecule of between 2:1 and 40:1. In some embodiments, the target-binding moiety, Y, has a intraendosomal:extracellular K_{D} ratio for the target molecule of between 2:1 and 30:1. In some embodiments, the target-binding moiety, Y, has a intraendosomal:extracellular K_{D} ratio for the target molecule of between 2:1 and 20:1. In some embodiments, the target-binding moiety, Y, has a intraendosomal:extracellular K_{D} ratio for the target molecule of between 10:1 and 1,000:1. In some embodiments, the target-binding moiety, Y, has a intraendosomal:extracellular K_{D} ratio for the target molecule of between 10:1 and 100:1. In some embodiments, the target-binding moiety, Y, has a pH 6.0:7.4 K_{D} ratio for the target molecule of between 2:1 and 10,000:1. In some embodiments, the target-binding moiety, Y, has a pH 6.0:7.4 K_{D} ratio for the target molecule of between 2:1 and 1,000:1. In some embodiments, the target-binding moiety, Y, has a pH 6.0:7.4 K_{D} ratio for the target molecule of between 2:1 and 500:1. In some embodiments, the target-binding moiety, Y, has a pH 6.0:7.4 K_{D} ratio for the target molecule of between 2:1 and 100:1. In some embodiments, the target-binding moiety, Y, has a pH 6.0:7.4 K_{D} ratio for the target molecule of between 2:1 and 50:1. In some embodiments, the target-binding moiety, Y, has a pH 6.0:7.4 K_{D} ratio for the target molecule of between 2:1 and 40:1. In some embodiments, the target-binding moiety, Y, has a pH 6.0:7.4 K_{D} ratio for the target molecule of between 2:1 and 30:1. In some embodiments, the target-binding moiety, Y, has a pH 6.0:7.4 K_{D} ratio for the target molecule of between 2:1 and 20:1. In some embodiments, the target-binding moiety, Y, has a pH 6.0:7.4 K_{D} ratio for the target molecule of between 10:1 and 1,000:1. In some embodiments, the target-binding moiety, Y, has a pH 6.0:7.4 K_{D} ratio for the target molecule of between 10:1 and 100:1.

In some embodiments, the target-binding moiety, Y, has a higher k_{off} rate for the target molecule intraendosomally than extracellularly. In some embodiments, the target-binding moiety, Y, has a higher k_{off} rate for the target molecule at an intraendosomal pH than at an extracellular pH. In some embodiments, the target-binding moiety, Y, is mutated so as to have a higher k_{off} rate for the target molecule at pH 6.0 than the corresponding wild type. In some embodiments, the target-binding moiety, Y, has a k_{off} rate for the target molecule intraendosomally of between 10⁻⁴ and 10⁻¹ (l/s). In some embodiments, the target-binding moiety, Y, has a k_{off} rate for the target molecule intraendosomally of between 10⁻³ and 10⁻¹ (l/s). In some embodiments, the target-binding moiety, Y, has a k_{off} rate for the target molecule intraendosomally of between 10⁻² and 10⁻¹ (l/s).

In some embodiments, the antibody or antibody fragment specifically binds to a cancer antigen.

In some embodiments, the antibody or antibody fragment specifically binds to a hepatocyte antigen.

In some embodiments, the antibody or antibody fragment specifically binds to an antigen presented on a macrophage.

In some embodiments, the antibody or antibody fragment specifically binds to an intact complement or a fragment thereof. In some embodiments, the antibody or antibody fragment specifically binds to one or more immunodominant epitope(s) within intact complement or a fragment thereof.

In some embodiments, the antibody or antibody fragment specifically binds to a cell surface receptor. In some embodiments, the antibody or antibody fragment specifically binds to a cell surface receptor ligand.

In some embodiments, the antibody or antibody fragment specifically binds to an epidermal growth factor (EGF) protein, e.g., a human EGF. In some embodiments, the antibody or antibody fragment specifically binds to one or more immunodominant epitope(s) within an EGF protein.

In some embodiments, the antibody or antibody fragment specifically binds to an epidermal growth factor receptor (EGFR) protein, e.g., a human EGFR. In some embodiments, the antibody or antibody fragment specifically binds to one or more immunodominant epitope(s) within an EGFR protein. In some embodiments, the antibody or antibody fragment comprises the CDRs present in cetuximab. In some embodiments, the antibody or antibody fragment includes the variable light chain and variable heavy chain present in cetuximab. In some embodiments, the antibody is cetuximab. In some embodiments, the antibody or antibody fragment includes the CDRs present in matuzumab. In some embodiments, the antibody or antibody fragment includes the variable light chain and variable heavy chain present in matuzumab. In some embodiments, the antibody is matuzumab.

In some embodiments, the antibody or antibody fragment specifically binds to vascular endothelial growth factor (VEGF) protein, e.g., human VEGF protein. In some embodiments, the antibody or antibody fragment specifically binds to one or more immunodominant epitope(s) within a VEGF protein.

In some embodiments, the antibody or antibody fragment specifically binds to a vascular endothelial growth factor receptor (VEGFR) protein, e.g., human VEGFR protein. In some embodiments, the antibody or antibody fragment specifically binds vascular endothelial growth factor receptor 2 (VEGFR2) protein, e.g., a human VEGFR2 protein. In some embodiments, the antibody or antibody fragment specifically binds a vascular endothelial growth factor receptor 3 (VEGFR3) protein, e.g., a human VEGFR3 protein. In some embodiments, the antibody or antibody fragment specifically binds to one or more immunodominant epitope(s) within a VEGFR protein, a VEGFR2 protein or a VEGFR3 protein.

In some embodiments, the antibody or antibody fragment specifically binds to a fibroblast growth factor (FGF), e.g., a human FGF. In some embodiments, the antibody or antibody fragment specifically binds to one or more immunodominant epitope(s) within a FGF protein.

In some embodiments, the antibody or antibody fragment specifically binds to a fibroblast growth factor receptor (FGFR), e.g., a human FGFR. In some embodiments, the antibody or antibody fragment specifically binds fibroblast growth factor receptor 2 (FGFR2) protein, e.g., a human FGFR2 protein, for example, a FGFR2b protein. In some embodiments, the antibody or antibody fragment specifically binds a fibroblast growth factor receptor 3 (FGFR3) protein, e.g., a human FGFR3 protein. In some embodiments, the antibody or antibody fragment specifically binds to one or more immunodominant epitope(s) within a FGFR protein, a FGFR2 protein or a FGFR3 protein.

In some embodiments, the antibody specifically binds to a receptor tyrosine kinase cMET protein. In some embodiments, the antibody specifically binds to one or more immunodominant epitope(s) within a receptor tyrosine kinase cMET protein.

In some embodiments, the antibody specifically binds to a CD47 protein, e.g., a human CD47 protein. In some embodiments, the antibody specifically binds to one or more immunodominant epitope(s) within a CD47 protein.

In some embodiments, the antibody specifically binds to an immune checkpoint inhibitor. In some embodiments, the antibody binds to one or more immunodominant epitope(s) within an immune checkpoint inhibitor. In some embodiments, the antibody specifically binds to a programmed death protein, e.g., a human PD-1. In some embodiments, the antibody specifically binds to one or more immunodominant epitope(s) within PD-1 protein.

In some embodiments, the antibody specifically binds to a programmed death ligand-1 (PD-L1) protein, e.g., a human PD-L1. In some embodiments, the antibody specifically binds to one or more immunodominant epitope(s) within PD-L1 protein.

In some embodiments, the antibody binds to TIM3. In some embodiments, the antibody binds to one or more immunodominant epitope(s) within TIM3.

In some embodiments, the antibody specifically binds to a lectin. In some embodiments, the antibody specifically binds to one or more immunodominant epitope(s) within a lectin. In some embodiments, the antibody binds to SIGLEC. In some embodiments, the antibody binds to one or more immunodominant epitope(s) within SIGLEC. In some embodiments, the antibody binds to a cytokine receptor. In some embodiments, the antibody binds to a one or more immunodominant epitope(s) within cytokine receptor. In some embodiments, the antibody binds to sIL6R. In some embodiments, the antibody binds to one or more immunodominant epitope(s) within sIL6R. In some embodiments, the antibody binds to a cytokine. In some embodiments, the antibody binds to one or more immunodominant epitope(s) within a cytokine. In some embodiments, the antibody binds to MCP-1, TNF (e.g., a TNF-alpha), IL1a, IL1b, IL4, IL5, IL6, IL12/IL23, IL13, IL17 or p40. In some embodiments, the antibody binds to one or more immunodominant epitope(s) within MCP-1, TNF (e.g., a TNF-alpha), IL1a, IL1b, IL4, IL5, IL6, IL12/IL23, IL13, IL17 or p40.

In some embodiments, the antibody binds to a major histocompatibility protein (e.g., a MHC class I or class II molecule). In some embodiments, the antibody binds to one or more immunodominant epitope(s) within a major histocompatibility protein (e.g., a MHC class I or class II molecule). In some embodiments, the antibody binds to beta 2 microglobulin. In some embodiments, the antibody binds to one or more immunodominant epitope(s) within beta 2 microglobulin. In some embodiments, the antibody binds IgE. In some embodiments, the antibody is omalizumab. In some embodiments, the antibody is a mutant form of omalizumab. In some embodiments, the antibody is ligelizumab. In some embodiments, the antibody is a mutant form of ligelizumab. In some embodiments, the antibody binds hC5. In some embodiments, the antibody is eculizumab or a mutant thereof. In some embodiments, the antibody is ALXN1210 or a mutant thereof.

In some embodiments, the antibody is omalizumab, ligelizumab, eculizumab, ALXN1210, or a mutant thereof. What is meant by mutant is that the antibody retains at least about 90% or 95% or 97% of the functionality or binding affinity of its intended target compared to the wild-type antibody but comprises one or more mutations as described herein.

In some embodiments, the antibody is omalizumab or a mutant thereof. The sequences of wild-type omalizumab is publicly known. For instance, the DrugBank Accession No. for omalizumab is DB00043 (go.drugbank.com/drugs/DB00043). In some embodiments, omalizumab includes one or more of the following mutations: S35H (LC), Y57H (LC), L443C (HC), M252Y (HC), S254T (HC), and T256E (HC). In some embodiments, omalizumab includes the following mutations: S35H (LC), Y57H (LC), L443C (HC), M252Y (HC), S254T (HC), and T256E (HC).

In some embodiments, the antibody is ligelizumab or a mutant thereof. The sequences of wild-type ligelizumab are publicly known. For instance, the KEGG Entry ID for ligelizumab is D11761 (www.kegg.jp/entry/D11761). In some embodiments, ligelizumab includes one or more of the following mutations: W33H (HC), Y50H (LC), N100bH (HC), W94H (LC), L443C (HC), M252Y (HC), S254T (HC), and T256E (HC). In some embodiments, ligelizumab includes the following mutations: W33H (HC), Y50H (LC), N100bH (HC), W94H (LC), L443C (HC), M252Y (HC), S254T (HC), and T256E (HC).

In some embodiments, the antibody is eculizumab or a derivative thereof. The sequences of wild-type eculizumab is publicly known. For instance, the NCATS No. for eculizumab is A3ULPOF556 (drugs.ncats.io/substance/A3ULPOF556). In some embodiments, eculizumab includes one or more of the following mutations: L443C (HC), M252Y (HC), S254T (HC), and T256E (HC). In some embodiments, eculizumab includes the following mutations: L443C (HC), M252Y (HC), S254T (HC), and T256E (HC).

In some embodiments, the antibody is ALXN1210 or a derivative thereof. The sequences of wild-type ALXN1210 is publicly known. For instance, the DrugBank Accession No. for ALXN1210 is DB11580 (go.drugbank.com/drugs/DB11580). In some embodiments, ALXN1210 includes one or more of the following mutations: L443C (HC), M252Y (HC), S254T (HC), and T256E (HC). In some embodiments, ALXN1210 includes the following mutations: L443C (HC), M252Y (HC), S254T (HC), and T256E (HC).

### Targets

As summarized above, the bifunctional compounds of this disclosure can include an antibody (Y) that specifically binds a target molecule. The target molecule can be a cell surface molecule or an extracellular molecule.

In some embodiments of the compounds and methods of this disclosure, the target molecule is a cell surface molecule. By “cell surface molecule” is meant a target molecule associated with a cell membrane, e.g., because the molecule has a domain that inserts into or spans a cell membrane, e.g., a cell membrane- tethering domain or a transmembrane domain. The cell surface molecule may be any cell surface molecule which is desired for targeted degradation via the endosomal/lysosomal pathway. In some embodiments, the cell surface molecule is a cell surface receptor.

Cell surface receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1). In some embodiments, the cell surface molecule is CD71 (transferrin receptor). In certain aspects, the cell surface receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In some embodiments, the antibody (Y) specifically binds a cell surface molecule which mediates its effect not through a specific molecular interaction (and therefore is not susceptible to blocking), but rather through bulk biophysical or aggregate effects. A non-limiting example of such a cell surface molecule is a mucin. Examples of mucins include, but are not limited to, MUC1, MUC16, MUC2, MUC5AC, MUC4, CD43, CD45, GPIb, and the like.

In some embodiments, when antibody specifically binds a cell surface molecule, the cell surface molecule is present on a cancer cell. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the cell surface molecule present on the cancer cell is a tumor-associated antigen or a tumor-specific antigen. In certain aspects, when the antibody (Y) specifically binds a cell surface molecule, the cell surface molecule is present on an immune cell. In some embodiments, the cell surface molecule is present on an immune cell selected from a T cell, a B cell, a natural killer (NK) cell, a macrophage, a monocyte, a neutrophil, a dendritic cell, a mast cell, a basophil, and an eosinophil. In certain aspects, the cell surface molecule present on the immune cell is an inhibitory immune receptor. As used herein, an “inhibitory immune receptor” is a receptor present on an immune cell that negatively regulates an immune response. Examples of inhibitory immune receptors which may be inhibited according to the methods of the present disclosure include inhibitory immune receptors of the Ig superfamily, including but not limited to: CD200R, CD300a (IRp60; mouse MAIR-I), CD300f (IREM-1), CEACAM1 (CD66a), FeyRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR—B); LAIR-1, PECAM-1 (CD31), PILR-a (FDF03), SIRL-1, and SIRP-a. Further examples of inhibitory immune receptors which may be inhibited according to the methods of the present disclosure include sialic acid-binding Ig-like lectin (Siglec) receptors, e.g., Siglec 7, Siglec 9, and/or the like. Additional examples of inhibitory immune receptors which may be inhibited according to the methods of the present disclosure include C-type lectins, including but not limited to: CLEC4A (DCIR), Ly49Q and MICL. Details regarding inhibitory immune receptors may be found, e.g., in Steevels et al. (2011) Eur. J. Immunol. 41 (3):575-587. In some embodiments, the cell surface molecule present on the immune cell is a ligand of an inhibitory immune receptor. In certain aspects, the cell surface molecule present on the immune cell is an immune checkpoint molecule. Non-limiting examples of immune checkpoint molecules to which the moiety of interest (Y) may specifically bind include PD-1, PD-L1, CTLA4, TIM3, LAG3, TIGIT, and a member of the B7 family.

In some embodiments of the compounds and methods of this disclosure, the target molecule is an extracellular molecule. By “extracellular molecule” is meant a soluble molecule external to the cell membranes of any cells in the vicinity of the soluble molecule. The extracellular molecule may be any extracellular molecule which is desired for targeted degradation via the endosomal/lysosomal pathway.

In some embodiments, the extracellular molecule is a soluble target protein. In some embodiments, the extracellular molecule is a secreted protein that accumulates in disease (e.g., alpha-synuclein), a cholesterol carrier (e.g., ApoB), an infectious disease toxin (e.g., AB toxins, ESAT-6), an infectious particle (e.g., a whole virus, a whole bacterium, etc.), a clotting factor (e.g., Factor IX), the target of any FDA approved antibody that binds to an extracellular molecule (e.g., TNFalpha), any chemokine or cytokine (e.g., mediators of sepsis or chronic inflammation such at IL-1), a proteinaceous hormone (e.g., insulin, ACTH, etc.), a proteinaceous mediator of a mood disorder, a proteinaceous mediator of energy homeostasis (e.g., leptin, ghrelin, etc.), a proteinaceous allergen present in the bloodstream or an antibody against such an allergen (e.g., for peanut allergies), a proteinaceous toxin (e.g., snake venom hyaluronidase, etc.), an autoantibody, etc.

In some embodiments, the target molecule is an extracellular molecule that is an antibody, e.g., an antibody that specifically binds a cell surface molecule or different extracellular molecule. In some embodiments, the antibody is an autoantibody. In some embodiments, the target is a human immunoglobulin A(IgA). In some embodiments, the IgA is a particular antibody that plays a crucial role in the immune function of mucous membranes. In the blood, IgA interacts with an Fc receptor called CD89 expressed on immune effector cells, to initiate inflammatory reactions. Aberrant IgA expression has been implicated in a number of autoimmune and immune-mediated disorders. In some embodiments, the target is a human immunoglobulin G (IgG). The Fc regions of IgGs include a conserved N-glycosylation site at asparagine 297 in the constant region of the heavy chain. Various N-glycans can be attached to this site. The N-glycan IgG composition has been linked to several autoimmune, infectious and metabolic diseases. In addition, overexpression of IgG4 has been associated with IG4-related diseases. In some embodiments, the target is human immunoglobulin E (IgE). IgE is a type of immunoglobulin that plays an essential role in type I hypersensitivity, which can manifest into various allergic diseases and conditions.

In some embodiments, the extracellular molecule is a ligand for a cell surface receptor. Cell surface receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor b (TGF-b), including any particular subtypes of such cytokines), hormones, and the like. In certain aspects, the antibody (Y) specifically binds IgE.

### Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, PA Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable catrer.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### Uses and Methods

In one aspect, provided herein are methods of using the conjugates described herein to remove a polypeptide of interest (a target protein) from a cell's surface. In one aspect, provided herein are methods of using the conjugates described herein to remove a polypeptide of interest (a target protein) from the extracellular milieu. For example, in one embodiment, provided herein are methods of using the conjugates described herein to remove a polypeptide of interest (a target protein) from the surface of a cell by sequestering the target protein in the cell's lysosome. In another embodiment, provided herein are methods of using the conjugates described herein to remove a polypeptide of interest (a target protein) from the extracellular space (the extracellular milieu) of a cell by sequestering the target protein in the cell's lysosome. In another embodiment, provided herein are methods of using the conjugates described herein to remove a polypeptide of interest (a target protein) from the surface of a cell by sequestering the target protein in the cell's lysosome and degrading the target protein. In another embodiment, provided herein are methods of using the conjugates described herein to remove a polypeptide of interest (a target protein) from the extracellular space (the extracellular milieu) of a cell by sequestering the target protein in the cell's lysosome and degrading the target protein.

Removal of a target protein may refer to reduction, or depletion, of the target protein from the cell surface or from the extracellular space, or the extracellular milieu, that is, a reduction, or depletion, of the amount of the target protein on the cell surface or in the extracellular milieu.

In one aspect, provided herein are methods of using the conjugates described herein to sequester a polypeptide of interest (a target protein) in a cell's lysosome. In one aspect, provided herein are methods of using the conjugates described herein to sequester a polypeptide of interest (a target protein) in a cell's lysosome and to degrade the polypeptide of interest.

In one aspect, provided herein are methods of using the conjugates described herein to degrade a polypeptide of interest (a target protein).

In one aspect, provided herein are methods of depleting a polypeptide of interest (a target protein) described herein by degradation through a cell's lysosomal pathway.

In another aspect, provided herein are methods of depleting a polypeptide of interest (a target protein) described herein by administering to a subject in need thereof an effective amount of a conjugate or pharmaceutically acceptable salt described herein, or a pharmaceutical composition described herein. In certain embodiments, the subject is a mammal (e.g., human).

In one method is provided, a method of degrading a target molecule in a subject in need thereof, comprising administering an effective amount of a conjugate as described herein to the subject. In one embodiment, least 90% of the target is degraded at four days following the administration. In one embodiment, at least 90% of the target is degraded at seven days following the administration. In one embodiment, a super-stoichiometric target:conjugate ratio is degraded. In one embodiment, the ratio is about 5. In one embodiment, the ratio is about 10. In one embodiment, the ratio is 10 to 100. In one embodiment, the ratio is about 100 to 1000. In one embodiment, the target-binding moiety, Y, has a higher binding affinity for the target molecule extracellularly than intraendosomally. In one embodiment, the target is IgE. In one embodiment, the conjugate facilitates degradation of a molar excess of target relative to the conjugate. In one embodiment, the molar excess is about 5. In one embodiment, the molar excess is about 10. In one embodiment, the molar excess is 10 to 100. In one embodiment, the molar excess is about 100 to 1000.

### Definitions

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a” “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. In certain embodiments, a polypeptide can occur as a single chain or as two or more associated chains, e.g., may be present as a multimer, e.g., dimer, a trimer. The terms also encompass an amino acid polymer that has been modified naturally or by intervention; for example, by disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein in their broadest sense and includes certain types of immunoglobulin molecules comprising one or more antigen-binding domains that specifically bind to an antigen or epitope.

In a certain embodiments, an isolated antibody (e.g., monoclonal antibody) described herein, or an antigen-binding fragment thereof, which specifically binds to a protein of interest, for example, EGFR, is conjugated to one or more lysosomal targeting moieties, for example, via a linker.

An “antigen” is a moiety or molecule that contains an epitope to which an antibody can specifically bind. As such, an antigen is also is specifically bound by an antibody. In a specific embodiment, the antigen, to which an antibody described herein binds, is a protein of interest, for example, EGFR (e.g., human EGFR), or a fragment thereof, or for example, an extracellular domain of EGFR (e.g., human EGFR).

The terms “clear,” “degrade,” “remove” and their respective cognates refer to degradation of a target or protein of interest, for example in a lysosome or late endosome. The terms “clear,” or “clearance” and other cognates may also refer to removal of a target (or protein of interest) from the extracellular environment, for example from serum or the media surrounding a cell.

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, ID), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

An antibody specifically includes, but is not limited to, full length antibodies (e.g., intact immunoglobulins), antibody fragments, monoclonal antibodies, polyclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgAQ1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, conjugate function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. Antibody fragments suitable for use in the compounds of this disclosure include, for example, Fv fragments, Fab fragments, F(ab′)₂ fragments, Fab′ fragments, scFv (sFv) fragments, and scFv-Fc fragments.

“Polynucleotide” or “nucleic acid,” as used interchangeably herein, and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. The nucleic acid molecule may be an aptamer.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides, polypeptides and virus particles of interest are well-known in the art and include, for example, ion-exchange chromatography, affinity chromatography and sedimentation according to density.

The terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in of tumor burden).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, camer, and adjuvant.

A “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intrathecal, intramuscular, subcutaneous, and the like.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)n- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, -SO2-alkyl, -SO2-aryl, -SO2-heteroaryl, and —NRaRb, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group refers to an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl”, unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” refers to a multi-valent (e.g., di-radical alkyl group, tri-radical alkyl group, tetra-radical alkyl group, etc.). Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” refer to di-radical alkenyl, alkynyl, aryl, aralkyl, and alkaryl groups, respectively.

In some embodiments, such as in branched constructs, the “alkylene” refers to a multi-valent (e.g., di-valent alkyl group, tri-valent alkyl group, tetra-valent alkyl group, etc.). Similarly, the terms “alkenylene,” “alkynylene,” “arylene,” “aralkylene,” and “alkarylene” can refer to multi-valent alkenyl, multi-valent alkynyl, multi-valent aryl, multi-valent aralkyl, and multi-valent alkaryl groups, respectively.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocycloalkyl” refers to a cycloalkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl and —SO₂— heteroaryl, and trihalomethyl.

The terms “heterocycle,” “heterocyclic” and “heterocyclyl” refer to a saturated or unsaturated group having a single ring or multiple condensed rings, including fused bridged and spiro ring systems, and having from 3 to 15 ring atoms, including 1 to 4 hetero atoms. These ring heteroatoms are selected from nitrogen, sulfur and oxygen, wherein, in fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C≡N), isocyanato (—O—N+≡C—), isothiocyanato (—S—C≡N), azido (—N═N+═N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O-)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)⁴; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other atypical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, -O⁻M⁺, —OR⁷⁰, —SR⁷⁰, -S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, -CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not -O⁻M⁺, —OR⁷⁰, —SR⁷⁰, or -S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, -O⁻M⁺, —OR⁷⁰, —SR⁷⁰, -S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In some embodiments, the term “optionally substituted” means that a group has 0-5, or 0-3, or 1, or 2, or 3, substituents independently selected from —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂OR⁷⁰, —OSO₂R⁷⁰, —OSO₂OR⁷⁰, —P(O)(OR⁷⁰)₂, —C(O)R⁷⁰, —C(S)R⁷⁰, —C(NR⁷⁰)R⁷ 0, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —O C(S)OR⁷⁰, —NR⁷⁰C(O)R⁷⁰, —NR⁷⁰C(S)R⁷⁰, —NR⁷⁰CO₂R⁷⁰, —NR⁷⁰C(S)OR⁷⁰, —NR⁷⁰C(O)NR⁸⁰R⁸⁰, —NR⁷⁰C (NR⁷⁰)R⁷⁰ and —NR⁷⁰C(NR⁷⁰)NR⁸⁰R⁸⁰, where R⁶⁰ is selected from the group consisting of alkyl, haloalkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

Definitions of other terms and concepts appear throughout the detailed description.

### Numbered Embodiments

Embodiment 1. A target binding conjugate of formula (I):

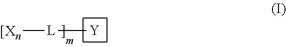

or a prodrug thereof, or a pharmaceutically acceptable salt thereof, wherein:
X is a moiety that binds to a lysosomal targeting molecule;
n is 1 to 20;
L is a linker;
m is 1 to 10; and
Y is an antibody that specifically binds a cell surface target molecule or extracellular target molecule;
wherein the conjugate is configured to exhibit extended activity and/or super-stoichiometric clearance of a target in a biological system.

Embodiment 2. The conjugate of Embodiment 1, wherein Y is selected from a human antibody, humanized antibody, or chimeric antibody.

Embodiment 3. The conjugate of Embodiment 1 or 2, wherein Y is an IgG antibody.

Embodiment 4. The conjugate of any one of Embodiments 1 to 3, wherein Y comprises an Fc region.

Embodiment 5. The conjugate of Embodiment 4, wherein the Fc region comprises one or more mutations that impart increased binding affinity for FcRn as compared to a reference Fc region lacking the one or more mutations.

Embodiment 6. The conjugate of Embodiment 5, wherein the Fc region comprises one or more mutations selected from amino acid substitutions methionine (Met) to tyrosine (Tyr), serine (Ser) to threonine (Thr), and threonine (Thr) to glutamic acid (Glu).

Embodiment 7. The conjugate of any one of the preceding Embodiments, wherein Y has a binding affinity for the target molecule that is calcium dependent and/or pH dependent.

Embodiment 8. The conjugate of Embodiment 7, wherein Y has higher binding affinity for the cell surface target molecule or extracellular target molecule at neutral pH than at low pH.

Embodiment 9. The conjugate of any one of the preceding Embodiments, wherein L is covalently linked to Y via a site-specific mutation.

Embodiment 10. The conjugate of Embodiment 9, wherein the site-specific mutation comprises a cysteine (Cys).

Embodiment 11. The conjugate of Embodiment 9 or 10, wherein the site-specific mutation is selected from an alanine (Ala) to cysteine (Cys) substitution, a lysine (Lys) to cysteine (Cys) substitution, a serine (Ser) to cysteine (Cys) substitution, a valine (Val) to cysteine (Cys) substitution, and a leucine (Leu) to cysteine (Cys) substitution.

Embodiment 12. The conjugate of any one of Embodiments 9 to 11, wherein the site-specific mutation is a leucine (Leu) to cysteine (Cys) substitution.

Embodiment 13. The conjugate of any one of the preceding Embodiments, wherein Y specifically binds a cell surface target molecule associated with a disease or condition of interest.

Embodiment 14. The conjugate of Embodiment 13, wherein the cell surface target molecule is a cell surface receptor.

Embodiment 15. The conjugate of any one of Embodiments 1 to 12, wherein Y specifically binds an extracellular target molecule associated with a disease or condition of interest.

Embodiment 16. The conjugate of Embodiment 15, wherein the extracellular target molecule is a ligand for a cell surface receptor.

Embodiment 17. The conjugate of Embodiment 15, wherein the extracellular target molecule is an antibody.

Embodiment 18. The conjugate of any one of Embodiments 1 to 18, wherein m is 1 to 6.

Embodiment 19. The conjugate of Embodiment 18, wherein m is 1.

Embodiment 20. The conjugate of Embodiment 18, wherein m is 2.

Embodiment 21. The conjugate of any one of Embodiments 1 to 20, wherein n is 1 to 20.

Embodiment 22. The conjugate of Embodiment 21, wherein n is 1.

Embodiment 23. The conjugate of Embodiment 21, wherein n is 2.

Embodiment 24. The conjugate of any one of Embodiments 1 to 21, wherein n is 3.

Embodiment 25. The compound of any one of Embodiments 1 to 24, wherein n is 1, and L comprises a linear linker having a backbone of 20 or more consecutive atoms covalently linking X to Y.

Embodiment 26. The compound of any one of Embodiments 1 to 24, wherein n is 2 or 3, and L is a branched linker that covalently links the X moieties to Y.

Embodiment 27. The compound of any one of Embodiments 1 to 26, wherein L is of formula (XI):

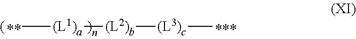

wherein
L¹ and L³ are independently a linker, and L² is a branched linking moiety, wherein L¹ to L³ together provide a linear or branched linker between X and Y;
a, b and c are independently 0 or 1;
** represents the point of attachment to L¹ of X via Z¹; and
*** represents the point of attachment to Y;
   wherein:
when n is 1, a is 1, and b is 0;
when n is >1, a is 1, and b is 1.

Embodiment 28. The compound of Embodiment 27, wherein L¹ to L³ each independently comprise one or more linking moieties independently selected from —C₁₋₂₀-alkylene-, —NHCO—C₁₋₆-alkylene-, —CONH—C₁₋₆-alkylene-, —NH C₁₋₆-alkylene-, —NHCONH—C₁₋₆-alkylene-, - NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, 1,2,3-triazole, —NH—, and —NMe-, wherein each p is independently 1 to 50.

Embodiment 29. The conjugate of any one of Embodiments 28 to 29, wherein the conjugate is of formula (II):

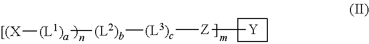

Embodiment 30. The conjugate of Embodiment 29, wherein Z is a residual moiety resulting from the covalent linkage of a chemoselective ligation group to a compatible group of Y.

Embodiment 31. The conjugate of Embodiment 29, wherein Z is a residual moiety resulting from the covalent linkage of a thiol-reactive chemoselective ligation group to one or more cysteine residue(s) of an antibody; or Z is a residual moiety resulting from the covalent linkage of an amine-reactive chemoselective ligation group to one or more lysine residue(s) of the antibody.

Embodiment 32. The conjugate of any one of Embodiments 1 to 31, wherein the lysosomal targeting molecule is selected from asialoglycoprotein receptor (ASGPR), cation independent mannose-6-phosphate receptor (CI-M6PR also referred to as M6PR), folate receptor, CD63, sortilin, IFITM3, molecules in the endosome/lysosome pathway, LIMP-1, and LIMP-2.

Embodiment 33. The conjugate of any one of Embodiments 1 to 32, wherein X is a moiety that binds to a cell surface asialoglycoprotein receptor (ASGPR).

Embodiment 34. The conjugate of Embodiment 33, wherein X is of formula:

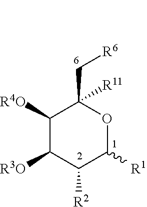

wherein:
   R¹ is selected from —OH, —OC(O)R, —C(O)NHR, —Z¹—*, and optionally substituted triazole, where R is optionally substituted C₁₋₆ alkyl or optionally substituted aryl;
   R² is selected from —NHCOCH₃, —NHCOCF₃, —NHCOCH₂CF₃, —OH, optionally substituted triazole, and —Z¹—*;
   R³ is selected from —H, —OH, —CH₃, —OCH₃, —OCH₂CH═CH and —Z¹—*;
   one of R¹ to R³ is —Z¹—*, wherein “*” represents a point of attachment of Z¹ to the linker (L);
   R⁴ and R⁵ are each independently selected from H, and a promoiety, or R⁴ and R⁵ are cyclically linked to form a promoiety;
   R¹¹ is H, or a group that forms a bridge to the 1-position carbon atom;
   Z¹ is a linking moiety selected from Z¹¹, optionally substituted Z¹¹-heteroaryl, optionally substituted Z¹¹-aryl, optionally substituted heteroaryl, optionally substituted aryl, optionally substituted alkyl, optionally substituted amide, optionally substituted sulfonamide, optionally substituted urea, and optionally substituted thiourea;
   Z¹¹ is selected from —O—, —S—, NR²¹—, and —C(R²²)₂,
   each R²¹ is independently selected from H, and optionally substituted (C₁-C₆)alkyl; and
   each R²² is independently selected from H, halogen (e.g., F) and optionally substituted (C₁-C₆)alkyl.

Embodiment 35. The compound of Embodiment 34, wherein each X is independently of formula:

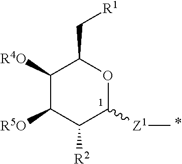

wherein:
R¹ is selected from —OH, —OC(O)R, and —C(O)NHR; and
R² is selected from —NHCOCH₃, —NHCOCF₃, and —NHCOCH₂CF₃.

Embodiment 36. The compound of Embodiment 35, wherein Z¹ is selected from —O—, —S—, and —C(R²²)₂—.

Embodiment 36a. The compound of Embodiment 35, wherein Z¹ is selected from —S—, and —C(R²²)₂—.

Embodiment 37. The compound of Embodiment 35, wherein Z¹ is Z¹¹—Ar, wherein Ar is optionally substituted heteroaryl or optionally substituted aryl.

Embodiment 38. The compound of Embodiment 37, wherein:
Z¹¹ is O, S, or C(R²²)₂; and
Ar is a monocyclic 5 or 6-membered heteroaryl or aryl.

Embodiment 39. The compound of Embodiment 38, wherein Z¹ is —C(R²²)₂-triazole-.

Embodiment 40. The compound of Embodiment 39, wherein Z¹ is or

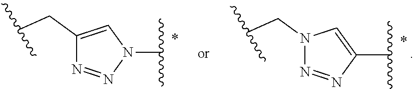

Embodiment 41. The compound of Embodiment 35, wherein Z¹ is monocyclic 5 or 6-membered heteroaryl or aryl.

Embodiment 42. The compound of Embodiment 41, wherein Z¹ is

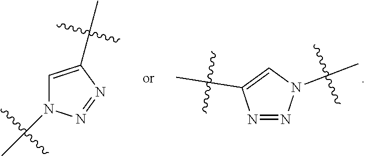 [SMILES: *c1cn(*)nn1 ; *c1cn(*)nn1]

Embodiment 43. The compound of Embodiment 34, wherein each X is independently of the formula:

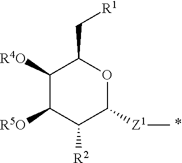

wherein R⁴ and R⁵ are each H.

Embodiment 44. The compound of Embodiment 43, wherein each X is independently of formula:

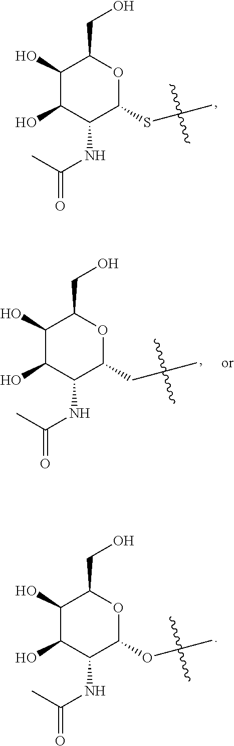 [SMILES: *S[C@H]1O[C@H](CO)[C@H](O)[C@H](O)[C@H]1NC(C)=O ; *C[C@H]1O[C@H](CO)[C@H](O)[C@H](O)[C@H]1NC(C)=O ; *O[C@H]1O[C@H](CO)[C@H](O)[C@H](O)[C@H]1NC(C)=O]

Embodiment 45. The compound of Embodiment 43, wherein Z¹ is selected from monocyclic 5 or 6-membered heteroaryl, monocyclic 5 or 6-membered aryl and Z¹¹—Ar, wherein Ar is optionally substituted heteroaryl or optionally substituted aryl.

Embodiment 46. The compound of Embodiment 45, wherein each X is

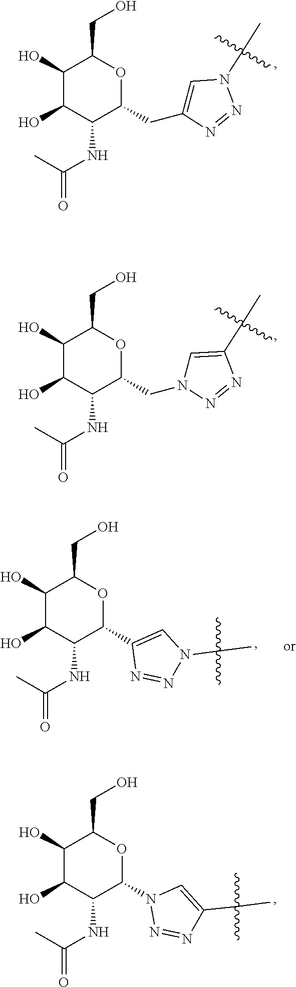 [SMILES: *n1cc(C[C@H]2O[C@H](CO)[C@H](O)[C@H](O)[C@H]2NC(C)=O)nn1 ; *c1cn(C[C@H]2O[C@H](CO)[C@H](O)[C@H](O)[C@H]2NC(C)=O)nn1 ; *n1cc([C@H]2O[C@H](CO)[C@H](O)[C@H](O)[C@H]2NC(C)=O)nn1 ; *c1cn([C@H]2O[C@H](CO)[C@H](O)[C@H](O)[C@H]2NC(C)=O)nn1]

Embodiment 47. The compound of Embodiment 34, wherein each X is independently of the formula:

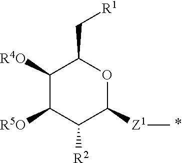

wherein R⁴ and R⁵ are each H.

Embodiment 48. The compound of Embodiment 47, wherein each X is selected from the following structures:

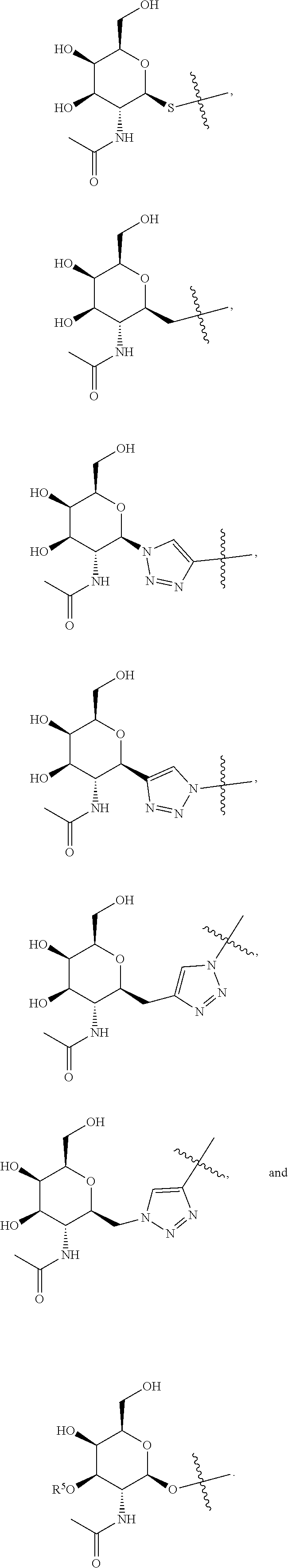

Embodiment 49. The compound of Embodiment 48, wherein n is 1 and X is

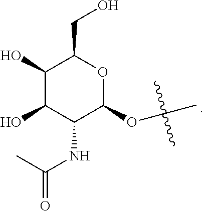 [SMILES: *O[C@@H]1O[C@H](CO)[C@H](O)[C@H](O)[C@H]1NC(C)=O]

Embodiment 50. The compound of Embodiment 34, wherein each X is independently of formula:

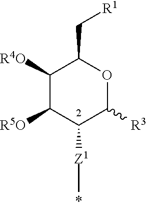

wherein:
R¹ is selected from —OH, —OC(O)R, and —C(O)NHR; and
R³ is selected from —H, —OH, —CH₃, —OCH₃, and —OCH₂CH═CH.

Embodiment 51. The compound of Embodiment 50, wherein R³ is H.

Embodiment 52. The compound of Embodiment 51, wherein each X is independently of formula:

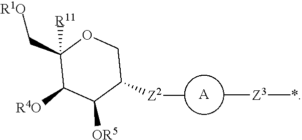

wherein:
Z² is absent or selected from —O—, —S—, NR²⁵—, —C(R²²)₂—, and optionally substituted Z¹²-alkyl;
ring A is absent or selected from a 5 or 6-membered optionally substituted aryl and a 5 or 6-membered optionally substituted heteroaryl;
Z³ is a linking moiety selected from Z¹², optionally substituted alkyl, optionally substituted Z¹²-alkyl, optionally substituted Z¹²-heteroaryl, optionally substituted Z¹²-aryl, optionally substituted heteroaryl, optionally substituted aryl, optionally substituted amide, optionally substituted sulfonamide, optionally substituted urea, and optionally substituted thiourea; and
Z¹² is selected from —CH₂O—, —O—, —S—, —NR²⁶—, and —C(R²²)₂—;
R²⁵ and R²⁶ are each independently selected from H, optionally substituted (C₁-C₆)alkyl (e.g., C₍₁₋₃₎-alkyl, such as methyl), and optionally substituted acyl; and
each R²² is independently selected from H, halogen (e.g., F) and optionally substituted (C₁-C₆)alkyl.

Embodiment 53. The compound of Embodiment 52, wherein each X is independently of one of formula:

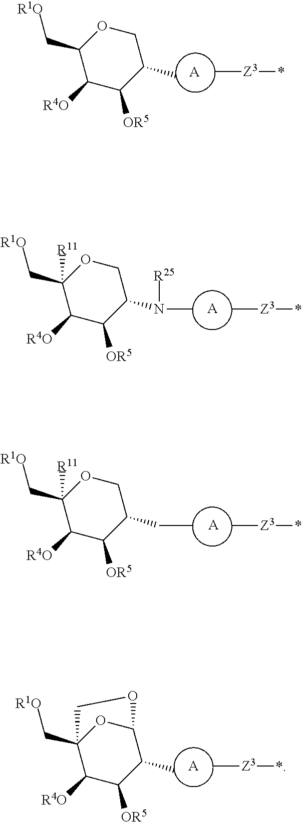

Embodiment 54. The compound of Embodiment 53, wherein each X is independently of one of formula:

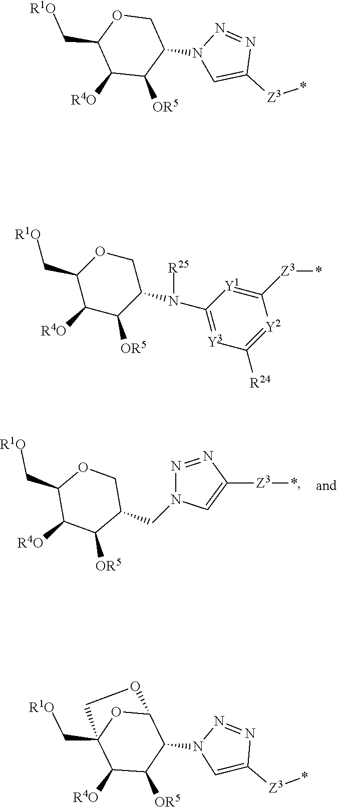

wherein:
Y¹-Y³ are each independently N or CR²⁷; and
R²⁴ and R²⁷ are each independently selected from H, optionally substituted C₍₁₋₆₎-alkyl, optionally substituted fluoroalkyl, and halogen.

Embodiment 55. The compound of Embodiment 54, wherein Z³ is selected from —O—, —CH₂O—, —OCH₂—, optionally substituted —OCH₂-heteroaryl, optionally substituted —OCH₂-aryl, optionally substituted —CH₂O-heteroaryl, and optionally substituted —CH₂O-aryl.

Embodiment 56. The compound of Embodiment 54 or 55, wherein X is independently one of the following structures:

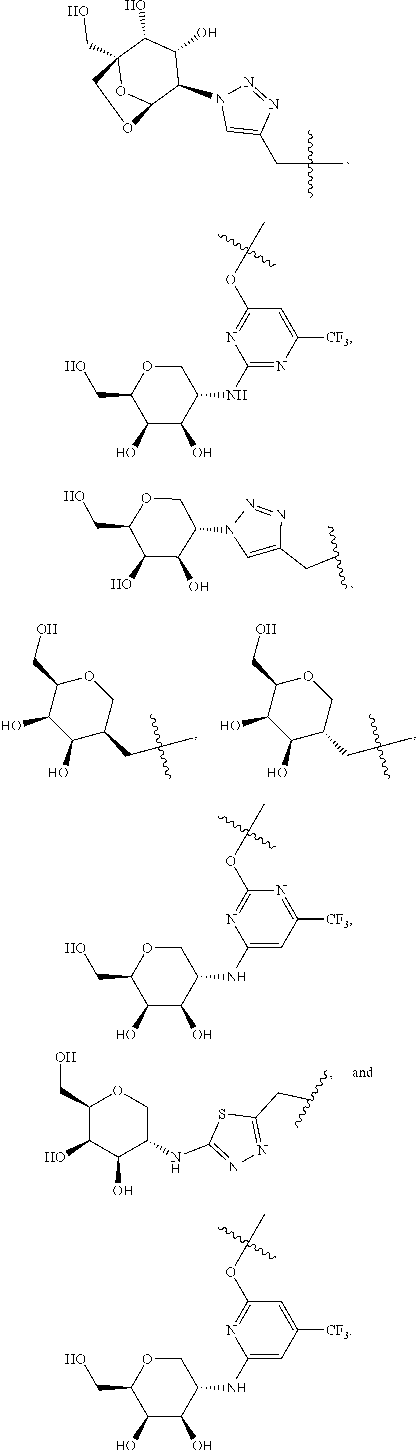

Embodiment 57. The compound of Embodiment 56, wherein X is:

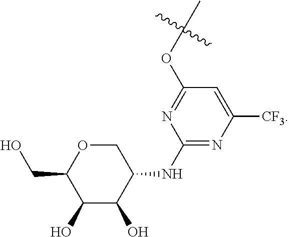 [SMILES: *Oc1cc(C(F)(F)F)nc(N[C@H]2CO[C@H](CO)[C@H](O)[C@@H]2O)n1]

Embodiment 58. The compound of Embodiment 34, wherein each X is independently of formula:

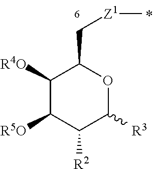

wherein:
R² is selected from —NHCOCH₃, —NHCOCF₃, and —NHCOCH₂CF₃; and
R³ is selected from —H, —OH, —CH₃, —OCH₃, and —OCH₂CH═CH.

Embodiment 59. The compound of Embodiment 58, wherein Z¹ is selected from —O—, —S—, —CONR²¹—, and optionally substituted —(C(R²²)₂)_{q}-heteroaryl, wherein q is 0 or 1.

Embodiment 60. The compound of Embodiment 58, wherein Z¹ is —O—.

Embodiment 61. The compound of Embodiment 58, wherein Z¹ is optionally substituted —(C(R²²)₂)_{q}-triazole wherein q is 0 or 1.

Embodiment 62. The compound of Embodiment 61, wherein Z¹ is

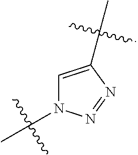 [SMILES: *c1cn(*)nn1 ; "."]

Embodiment 63. The compound of any one of Embodiments 33 to 62, wherein n is 1, and L comprises a linear linker having a backbone of 20 or more consecutive atoms covalently linking X to Y via Z¹.

Embodiment 64. The compound of any one of Embodiments 33 to 62, wherein n is 2 or more, and L is a branched linker that covalently links 2 or more X moieties to Y via the linking moiety Z¹.

Embodiment 65. The compound of any one of Embodiments 33 to 64, wherein L is of formula:

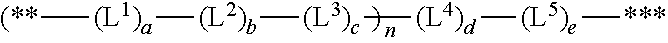

wherein
each L¹ to L⁵ is independently a linking moiety which together provide a linear or branched linker between Z¹ and Y;
a, b, c, d, and e are each independently 0, 1, or 2;
** represents the point of attachment to L¹ of X via Z¹; and
*** represents the point of attachment to Y,
wherein when n is >1, d is 1 or 2 and L4 is a branching moiety.

Embodiment 66. The compound of Embodiment 65, wherein L¹ to L⁵ each independently comprise one or more linking moieties independently selected from —C₁₋₂₀-alkylene-, —NHCO—C₁₋₆-alkylene-, —CONH—C₁₋₆-alkylene-, —NH—C₁₋₆-alkylene-, —NHCONH—C₁₋₆-alkylene-, - NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, 1,2,3-triazole, —NH—, and —NMe-, wherein each p is independently 1 to 50.

Embodiment 67. The compound of Embodiment 65 or 66, wherein L comprises repeating ethylene glycol moieties.

Embodiment 68. The compound of Embodiment 67, wherein L comprises 1 to 25 ethylene glycol moieties.

Embodiment 69. The compound of any one of Embodiments 65 to 68, wherein L comprises one or more 1,2,3-triazole linking moieties.

Embodiment 70. The compound of any one of Embodiments 65 to 69, wherein n is 1.

Embodiment 71. The compound of any one of Embodiments 65 to 69, wherein n is 2 or more.

Embodiment 72. The compound of Embodiment 71, wherein L⁴ is a branching moiety selected from

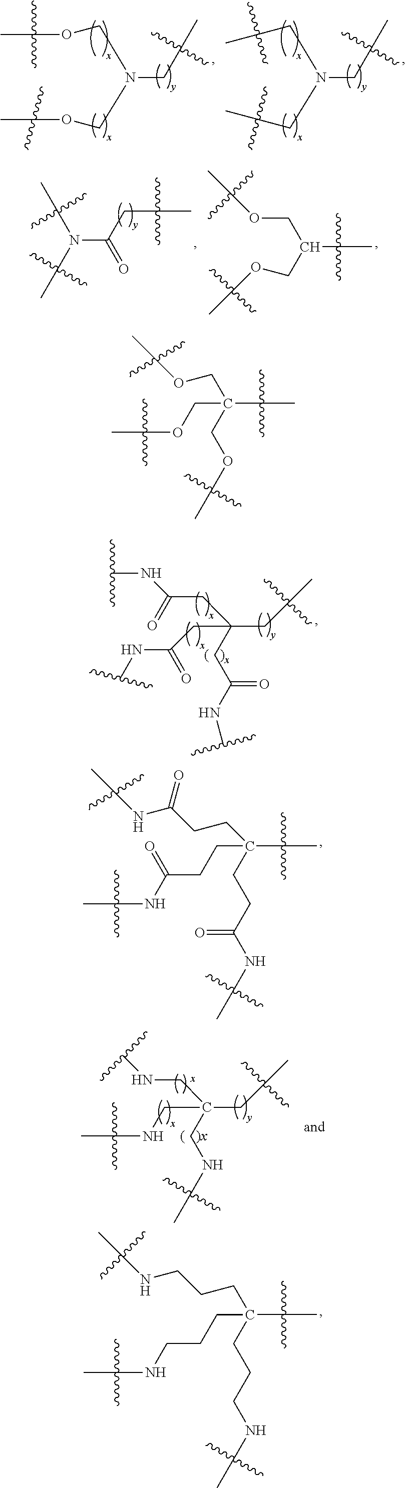

wherein each x and y are each independently 1 to 10.

Embodiment 73. The compound of any one of Embodiments 64 to 39, wherein L¹-L⁴ comprises a backbone of 14 or more consecutive atoms between X and the branching atom.

Embodiment 74. The compound of any one of Embodiments 65 to 73, wherein L⁵ comprises a backbone of 10 to 80 consecutive atoms.

Embodiment 75. The compound of Embodiment 74, wherein L⁵ comprises a linking moiety selected from (C₁₀-C₂₀-alkylene, or —(OCH₂CH₂)ₚ—, where p is 1 to 25.

Embodiment 76. The compound of any one of Embodiments 32 to 42, wherein the linker of formula (IIb) comprises a backbone of 20 to 100 consecutive atoms.

Embodiment 77. The compound of Embodiment 76, wherein the linker of formula (IIb) comprises a backbone of 25 or more consecutive atoms.

Embodiment 78. The compound of Embodiment 77, wherein the linker of formula (IIb) comprises a backbone of 30 or more consecutive atoms.

Embodiment 79. The compound of any one of Embodiments 34 to 78, wherein —Z¹-L¹-comprises a group selected from:

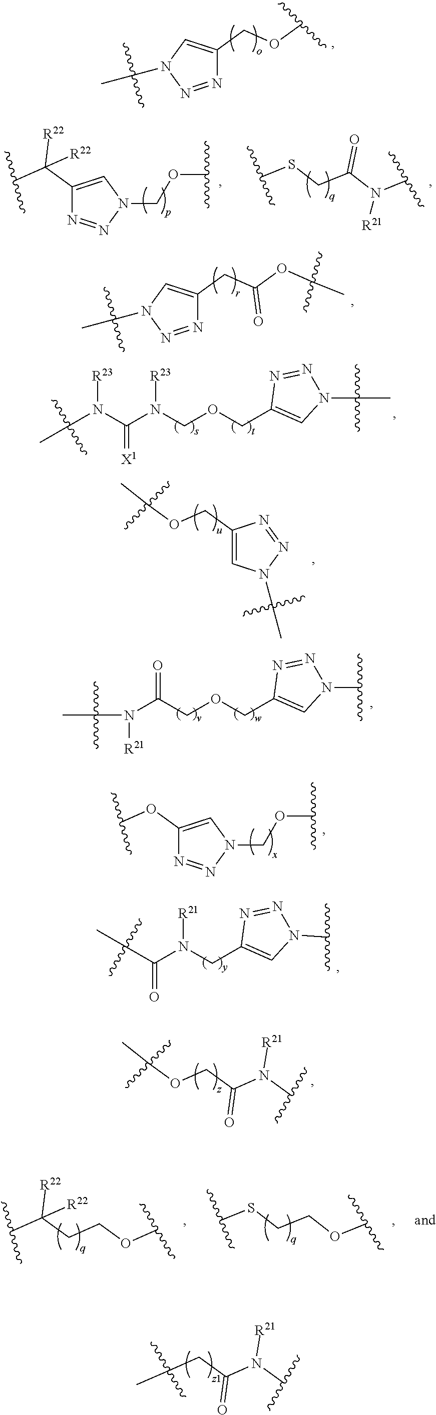

wherein:
each R²¹ is independently selected from H, and optionally substituted (C₁-C₆)alkyl;
each R²² is independently selected from H, halogen (e.g., F) and optionally substituted (C₁-C₆)alkyl; and
o, p, q, r, s, t, u, v, w, x, y, z and z1 are each independently 1 to 6.

Embodiment 80. The compound of Embodiment 79, wherein —Z¹-L¹- comprises a group selected from:

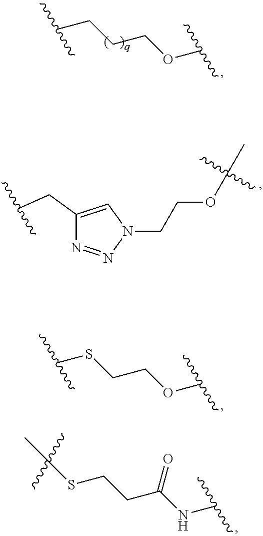

wherein q is 1 to 3.

Embodiment 81. The compound of any one of Embodiments 34 to 78, wherein —Z¹-L¹-comprises an optionally substituted —NH-heteroaryl-.

Embodiment 82. The compound of Embodiment 81, wherein —Z¹-L¹- comprises a group selected from:

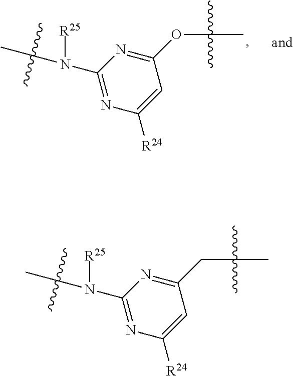

wherein:
each R²⁴ is independently selected from H, optionally substituted C₍₁₋₆₎-alkyl, optionally substituted fluoroalkyl, and halogen; and
each R²⁵ is independently selected from H, optionally substituted (C₁-C₆)alkyl, and optionally substituted acyl.

Embodiment 83. The conjugate of any one of Embodiments 1 to 32, wherein X is a moiety that binds to a CI-M6PR.

Embodiment 84. The conjugate of Embodiment 83, wherein X is of formula (IV):

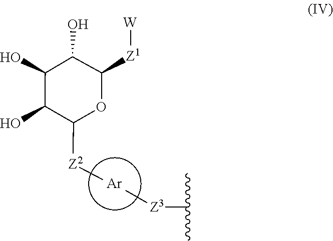

wherein:
W is a non-hydrolyzable hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen and optionally substituted (C₁-C₆)alkyl;
each A is independently an optionally substituted aryl or heteroaryl linking moiety; and
each Z³ is independently a linking moiety.

Embodiment 85. The compound of Embodiment 84, wherein Z² is S.

Embodiment 86. The compound of Embodiment 84 or 85, wherein W is phosphonate, thiophosphonate, carboxylic or malonic acid, or a salt thereof.

Embodiment 87. The compound of any one of Embodiments 84 to 86 wherein the compound comprises a M6PR binding moiety (X) of one of formula:

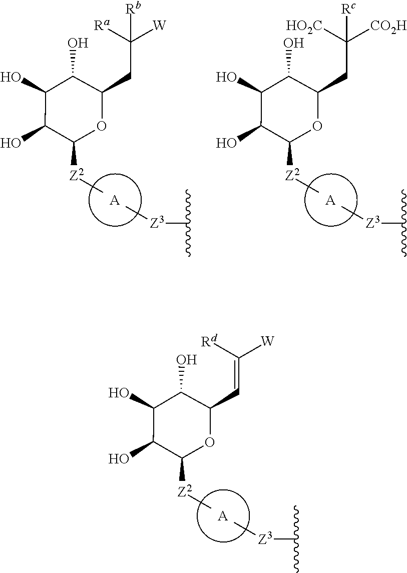

wherein R^{a}, R^{b}, R^{c} and R^{d} are independently H or F.

Embodiment 88. The compound of Embodiment 84, wherein the compound comprises a M6PR binding moiety (X) of one of formula:

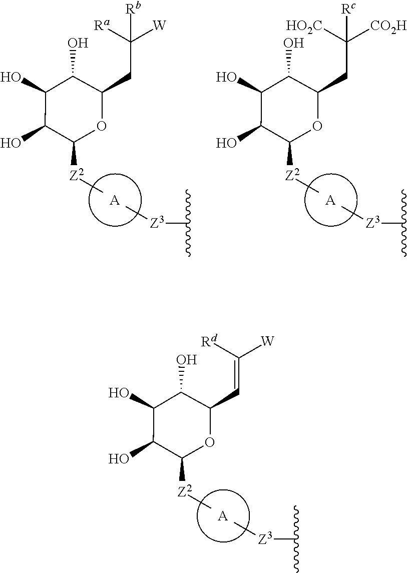

wherein R^{a}, R^{b}, R^{c} and R^{d} are independently H or F.

Embodiment 89. The compound of any one of Embodiments 84-88, wherein A is optionally substituted aryl or optionally substituted heteroaryl, preferably A is independently selected from optionally substituted phenyl, optionally substituted pyridyl, optionally substituted biphenyl, optionally substituted naphthalene, optionally substituted triazole and optionally substituted phenylene-triazole.

Embodiment 90. The compound of Embodiment 89, wherein A is selected from optionally substituted 1,4-phenylene, optionally substituted 1,3-phenylene, optionally substituted 2,5-pyridylene and triazole.

Embodiment 91. The compound of Embodiment 90, wherein A is selected from:

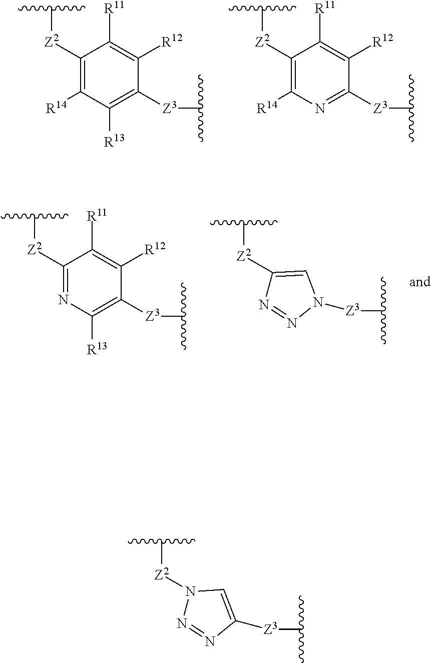

wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
R²⁵ is independently selected from H, and optionally substituted (C₁-C₆)alkyl.

Embodiment 92. The compound of any one of Embodiments 84-91, wherein A is optionally substituted fused bicyclic aryl or optionally substituted fused bicyclic heteroaryl.

Embodiment 93. The compound of Embodiment 92, wherein A is optionally substituted naphthalene or optionally substituted quinoline.

Embodiment 94. The compound of Embodiment 93, wherein A is selected from:

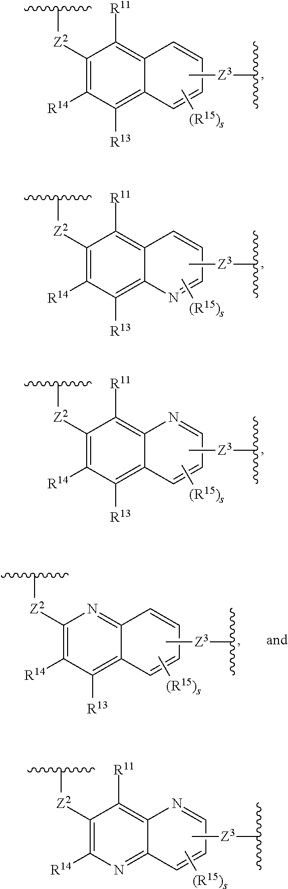

wherein:
R¹¹ and R¹³ to R¹⁴ is independently selected from H, halogen, OH, optionally substituted (C₁ —C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

Embodiment 95. The compound of Embodiment 94, wherein A is selected from:

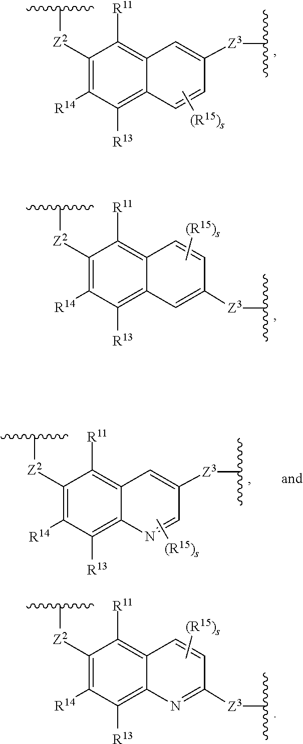

Embodiment 96. The compound of any one of Embodiments 84-90, wherein A is optionally substituted bicyclic aryl or optionally substituted bicyclic heteroaryl of following formula:

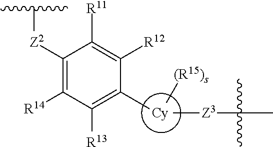

or a salt thereof,
wherein:
Cy is independently monocyclic aryl or monocyclic heteroaryl;
R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²⁵ is independently selected from H, and optionally substituted (C₁-C₆)alkyl.

Embodiment 97. The compound of Embodiment 96, wherein Cy is optionally substituted phenyl, and A is optionally substituted biphenyl of the formula:

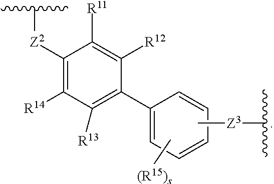

Embodiment 98. The compound of Embodiment 97, wherein A is selected from:

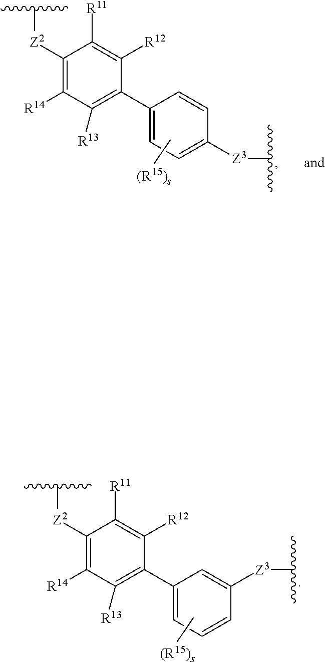

Embodiment 99. The compound of Embodiment 96, wherein Cy is triazole, and A is selected from:

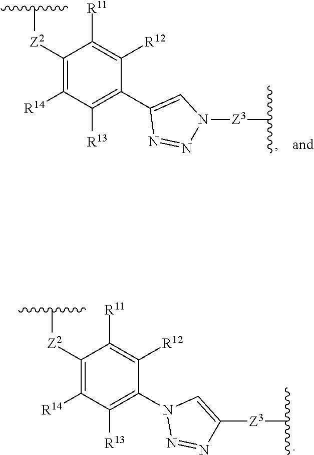

Embodiment 100. The compound of any one of Embodiments 90 to 99, wherein A is substituted with at least one OH substituent.

Embodiment 101. The compound of any one of Embodiments 94, and 96-100, wherein at least one of R¹¹ to R¹⁵ is OH (e.g., at least two are OH).

Embodiment 102. The compound of any one of Embodiments 94, and 96-100, wherein R¹¹ to R¹⁵ are each H.

Embodiment 103. The compound of any one of Embodiments 84 to 102, wherein:
Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—, wherein:
X¹ and X² are selected from O, S and NR²³; and
R²³ and R²⁴ are independently selected from H, C₍₁₋₃₎-alkyl (e.g., methyl) and substituted C₍₁₋₃₎-alkyl.

Embodiment 104. The compound of any one of Embodiments 84 to 103, wherein Z³ is

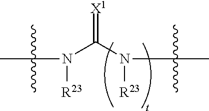

wherein:
X¹ is O or S;
t is 0 or 1; and
each R²³ is independently selected from H, C₍₁₋₃₎-alkyl (e.g., methyl) and substituted C₍₁₋₃₎-alkyl.

Embodiment 105. The compound of Embodiment 104, wherein Z³ is —NHC(═O)NH—.

Embodiment 106. The compound of any one of Embodiments 84 to 104, wherein -A-Z³— is selected from:

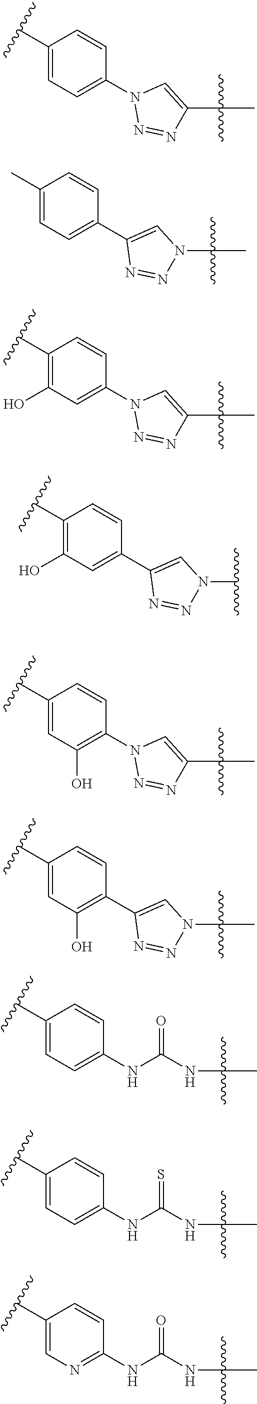

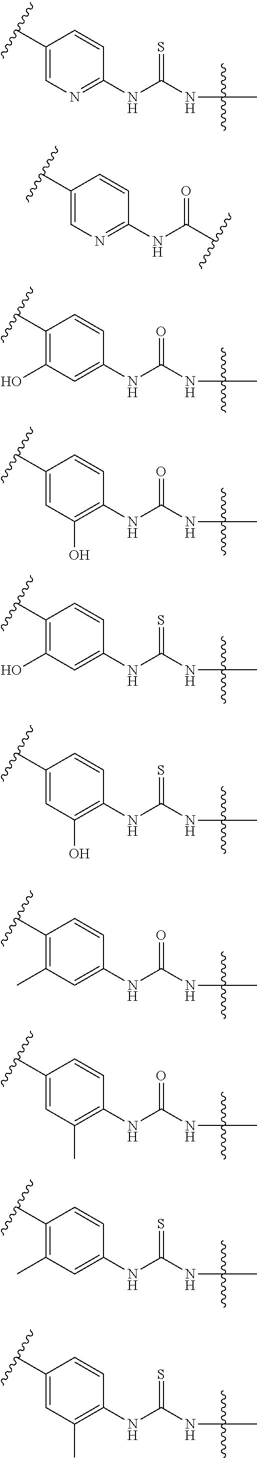

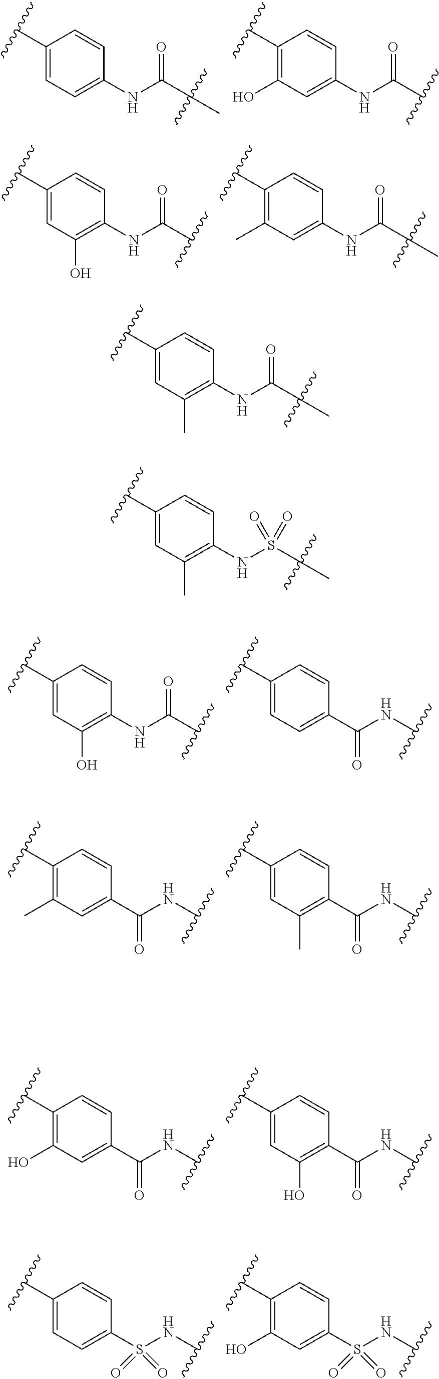

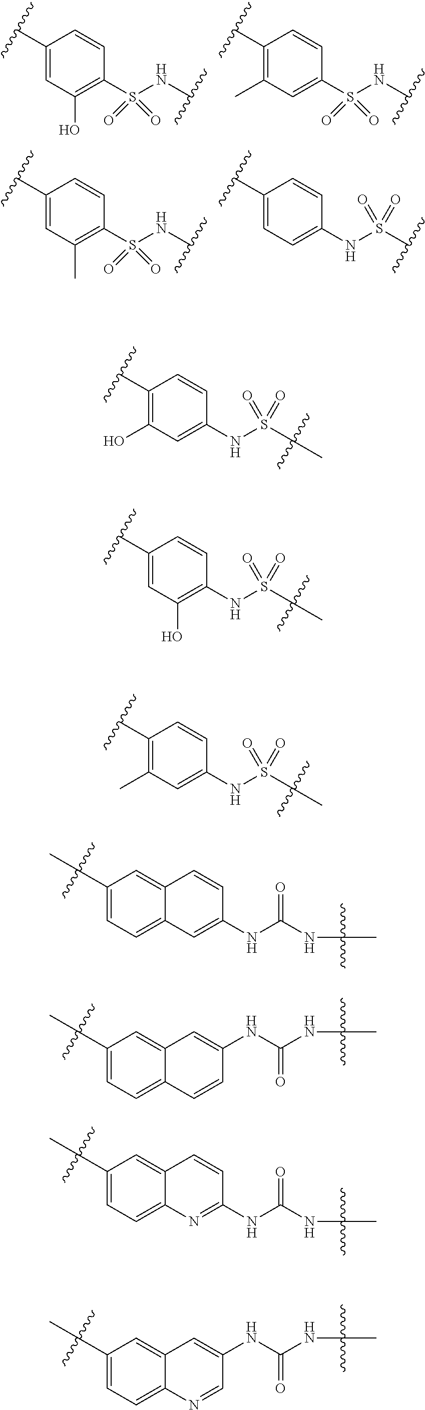

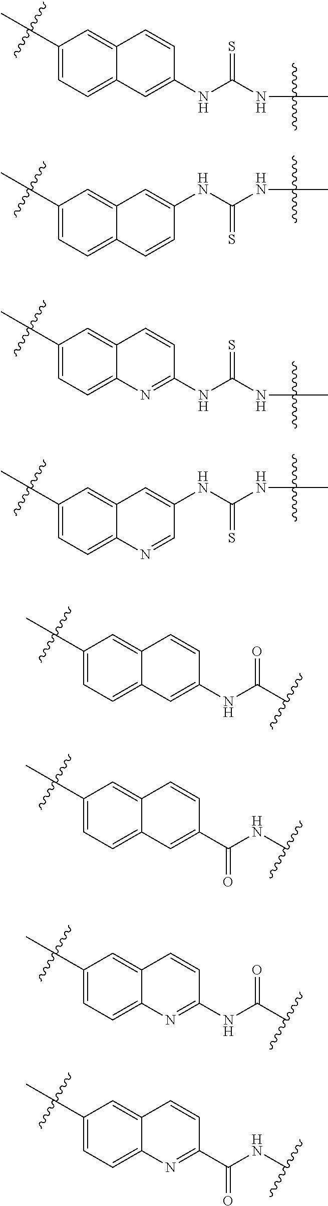

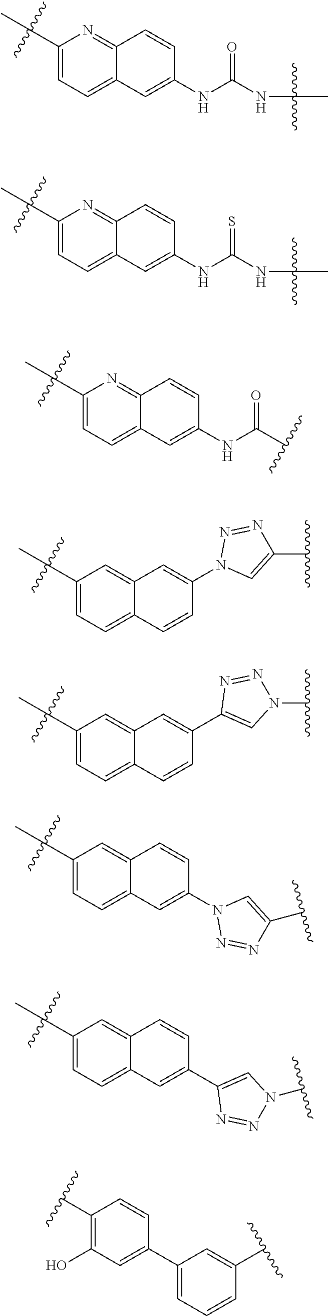

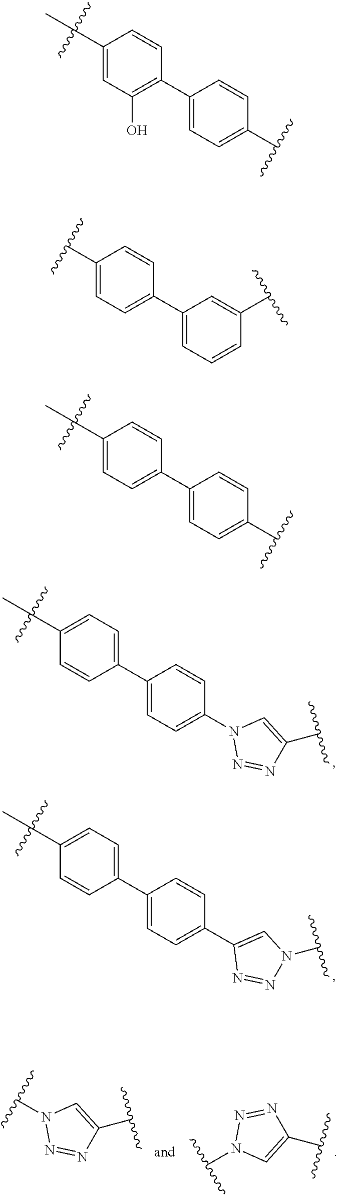

Embodiment 107. The compound of any one of Embodiments 84 and 87-106, wherein Z² is O.

Embodiment 108. The compound of any one of Embodiments 84-106, wherein Z² is S.

Embodiment 109. The compound of any one of Embodiments 84 and 87-106, wherein Z² is —NR²¹—.

Embodiment 110. The compound of any one of Embodiments 84 and 87-106, wherein Z² is —C(R²²)₂—, wherein each R²² is independently selected from H, halogen (e.g., F) and optionally substituted (C₁-C₆)alkyl.

Embodiment 111. The compound of Embodiment 110, wherein Z² is —CH₂— or —CF₂—.

Embodiment 112. The compound of any one of Embodiments 84 and 89-108, wherein —Z²—Ar—Z³— is

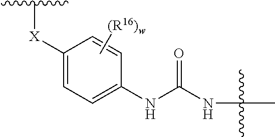

wherein:
X is O, S, —CH₂— or —CF₂;
R¹⁶ is OH; and
w is 0 to 4 (e.g., w is 0, 1, or 2).

Embodiment 113. The compound of Embodiment 112, wherein —Z²—Ar—Z³— is

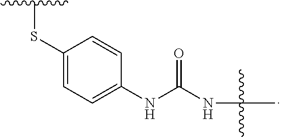 [SMILES: *NC(=O)Nc1ccc(S*)cc1]

Embodiment 114. The compound of any one of Embodiments 84 to 113, wherein the non-hydrolyzable hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —CR¹R²—P═O(OH)₂, —SO₂OH (i.e., —SO₃H), —S(O)OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, — NHSO₂NHR³, —NHC(O)NHS(O)₂R³, —NHSO₂R³, —NHSO₃H,

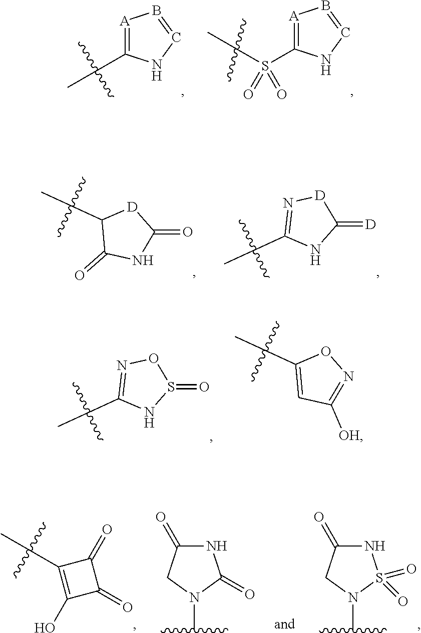

or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

Embodiment 115. The compound of Embodiment 114, wherein W is selected from —P═O(OH)₂, —P═S(OH)₂, —P═O(SH)(OH), —P═S(SH)(OH), —COOH and —CH(COOH)₂, or a salt thereof.

Embodiment 116 The compound of any one of Embodiments 84 to 115, wherein Z¹ is —(C(R²²)₂), -, wherein each R²² is independently selected from H, halogen (e.g., F) and optionally substituted (C₁-C₆)alkyl, and j is 1 to 3.

Embodiment 117 The compound of Embodiment 116, wherein Z¹ is —(CH₂)₂—, —CH₂—CF₂—, —CH₂—CHF—.

Embodiment 118. The compound of Embodiment 116, wherein Z¹ is —CH₂— or —CF₂—.

Embodiment 119. The compound of any one of Embodiments 84 to 115, wherein Z¹ is —CH═CH—.

Embodiment 120. The compound of Embodiment 116, wherein:
Z¹ is —(CH₂)₂—, —CH₂—CF₂— or —CH₂—CHF—; and
W is selected from —P═O(OH)₂, —P═S(OH)₂, —P═O(SH)(OH), —P═S(SH)(OH), and —COOH, or a salt thereof.

Embodiment 121. The compound of Embodiment 119, wherein:
Z¹ is —CH═CH—; and
W is selected from —P═O(OH)₂, —P═S(OH)₂, —P═O(SH)(OH), —P═S(SH)(OH), and —COOH, or a salt thereof.

Embodiment 122. The compound of Embodiment 120, wherein:
Z¹ is —CH₂—, or —CF₂—; and
W is —CH(COOH)₂, or a salt thereof.

Embodiment 123. The compound of any one of Embodiments 84 to 122, wherein n is 1 to 20 (e.g., 1 to 10, 1 to 6, or 1 to 3).

Embodiment 124. The compound of Embodiment 123, wherein n is 1.

Embodiment 125. The compound of Embodiment 124, wherein L comprises a linear linker having a backbone of 16 or more consecutive atoms covalently linking Z³ to Y (e.g., a backbone of 16-100, or 20-100 consecutive atoms).

Embodiment 126. The compound of Embodiment 123, wherein n is 2.

Embodiment 127. The compound of Embodiment 123, wherein n is 3.

Embodiment 128. A method of reducing levels of an extracellular target molecule in a biological system, the method comprising contacting the biological system with an effective amount of a conjugate according to any one of Embodiments 1 to 39, wherein the compound specifically binds the extracellular target molecule and specifically binds a lysosomal targeting molecule of cells in the biological system to facilitate cellular uptake and degradation of the extracellular target molecule.

Embodiment 129. The method of Embodiment 128, wherein the effective amount of the conjugate provides super-stoichiometric clearance of the target from the extracellular medium in the biological system.

Embodiment C-1. A conjugate that comprises a ligand moiety, X, conjugated via a linker, L, to a target-binding moiety, Y,
wherein
   the ligand moiety, X, binds a lysosomal targeting molecule extracellularly;
   the target-binding moiety, Y, binds a target molecule extracellularly,
   the target-binding moiety, Y, dissociates from the target molecule intraendosomally; and
   the conjugate is externalized from a cell.

Embodiment C-2. The conjugate of Embodiment C-1, wherein ligand moiety, X, remains bound to the lysosomal targeting molecule intraendosomally.

Embodiment C-3. The conjugate of Embodiment C-1 or C-2, wherein the target-binding moiety, Y, binds FcRn intraendosomally.

Embodiment C-4. The conjugate of any one of Embodiments C-1 to C-3, wherein the conjugate dissociates from the lysosomal targeting molecule intraendosomally.

Embodiment C-5. The conjugate of Embodiment C-1, wherein the lysosomal targeting molecule is ASGPR.

Embodiment C-6. The conjugate of Embodiment C-1, wherein Y is an antibody or antibody fragment.

Embodiment C-7. The conjugate of Embodiment C-2, wherein the conjugate is externalized from the cell via the lysosomal targeting molecule.

Embodiment C-8. The conjugate of Embodiment C-2, wherein the ligand moiety, X, has an equal binding affinity for the lysosomal targeting molecule extracellularly and intraendosomally.

Embodiment C-9. The conjugate of Embodiment C-2, wherein the ligand moiety, X, has an equal binding affinity for the lysosomal targeting molecule at an extracellular pH and at an intraendosomal pH.

Embodiment C-10. The conjugate of Embodiment C-2, wherein the ligand moiety, X, has an equal binding affinity for the lysosomal targeting molecule at an extracellular Ca²⁺ concentration and at an intraendosomal Ca²⁺ concentration.

Embodiment C-11. The conjugate of Embodiment C-3, wherein the conjugate is externalized from the cell via FcRn.

Embodiment C-12. The conjugate of Embodiment C-4, wherein the target-binding moiety, Y, binds FcRn intraendosomally.

Embodiment C-13. The conjugate of Embodiment C-12, wherein the conjugate is externalized from the cell via FcRn.

Embodiment C-14. The conjugate of Embodiment C-12, wherein the ligand moiety, X, has a higher binding affinity for the lysosomal targeting molecule extracellularly than intraendosomally.

Embodiment C-15. The conjugate of Embodiment C-12, wherein the ligand moiety, X, has a higher binding affinity for the lysosomal targeting molecule at an extracellular pH than at an intraendosomal pH.

Embodiment C-16. The conjugate of Embodiment C-12, wherein the ligand moiety, X, has a higher binding affinity for the lysosomal targeting molecule at an extracellular Ca²⁺ concentration than at an intraendosomal Ca²⁺ concentration.

Embodiment C-17. The conjugate of Embodiment C-12, wherein the target-binding moiety, Y, has a higher binding affinity for FcRn intraendosomally than extracellularly.

Embodiment C-18. The conjugate of Embodiment C-12, wherein the target-binding moiety, Y, has a higher binding affinity for FcRn at an intraendosomal pH than at an extracellular pH.

Embodiment C-19. The conjugate of Embodiment C-12, wherein the target-binding moiety, Y, has enhanced binding to FcRn relative to wild-type at an endosomal pH.

Embodiment C-20. The conjugate of Embodiment C-12, wherein the target-binding moiety, Y, has approximately equal binding affinity to FcRn extracellularly as wild-type IgG does extracellularly.

Embodiment C-21. The conjugate of Embodiment C-12, wherein the target-binding moiety, Y, has, at pH 7.4, approximately equal binding affinity to FcRn as wild-type IgG.

Embodiment C-22. The conjugate of Embodiment C-12, wherein Y is an antibody.

Embodiment C-22a. In any one of Embodiment C-1-C-22, the antibody or antibody fragment has a binding affinity for the target molecule that is calcium dependent.

Embodiment C-22b. In any one of Embodiment C-1-C-22a, the antibody or antibody fragment has one or more mutations that impart pH-dependent binding affinity for the target molecule.

Embodiment C-22c. In any one of Embodiment C-1-C-22b, the antibody or antibody fragment has been mutated from the wild-type with one or more histidine substitutions.

Embodiment C-22d. The conjugate of Embodiment C-22c, wherein the one or more histidine substitutions are located in the CDR region of the antibody or antibody fragment.

Embodiment C-22e. In any one Embodiment of C-1-C22d, wherein Y or the antibody is omalizumab or mutant thereof or ligelizumab or mutant thereof.

Embodiment C-22f. The conjugate of Embodiment, C-22, wherein the antibodies or fragment thereof can include one or more mutations from the following table:

| | |
|---|---|
| Omalizumab | S35H-LC, Y57H-LC |
| Ligelizumab | W33HHC/Y50H-N100bH- |
| | HC/W94H-LC |

Embodiment C-23. The conjugate of Embodiment C-1, wherein the target-binding moiety, Y, has a higher binding affinity for the target molecule extracellularly than intraendosomally.

Embodiment C-24. The conjugate of Embodiment C-1, wherein the target-binding moiety, Y, has a higher binding affinity for the target molecule at an extracellular pH than at an intraendosomal pH.

Embodiment C-25. The conjugate of Embodiment C-1, wherein the target-binding moiety, Y, has a pH 6.0:7.4 K_{D} ratio for the target molecule of between 2:1 and 10,000:1.

Embodiment C-26. The conjugate of Embodiment C-1, wherein the target-binding moiety, Y, has a higher k_{off} rate for the target molecule intraendosomally than extracellularly.

Embodiment C-27. The conjugate of Embodiment C-1, wherein the conjugate is capable of cycling for a period of hours to days. In o

Embodiment C-28. The conjugate of Embodiment C-1, wherein the target-binding moiety, Y is an antibody or antibody fragment that has been mutated from the wild-type with one or more histidine substitutions.

Embodiment C-29. The conjugate of any preceding Embodiment C-1 to C-28, wherein the conjugate is of formula (I′):

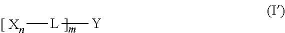

or a prodrug thereof, or a or a pharmaceutically acceptable salt thereof,
wherein:
n is 1 to 500;
m is 1 to 20;
X is the ligand moiety; and
Y is the target-binding moiety.

Embodiment C-29a. The conjugate of C-29, wherein n is 1, 2, or 3 and m is 1-3.

Embodiment C-30. The conjugate of any preceding Embodiment, wherein the lysosomal targeting molecule is a cell surface receptor that provides for internalization of the conjugate.

Embodiment C-31. The conjugate of any preceding Embodiment, wherein the lysosomal targeting molecule is selected from asialoglycoprotein receptor (ASGPR), cation independent mannose-6-phosphate receptor (CI-M6PR also referred to herein as M6PR), folate receptor, LDLR, CD63, sortilin, IFITM3, molecules in the endosome/lysosome pathway, LIMP-1, and LIMP-2.

Embodiment C-32. The conjugate of any preceding Embodiment, wherein X is a moiety that binds ASGPR or M6PR.

Embodiment C-33. The conjugate of any preceding Embodiment, wherein X is a moiety that binds ASGPR.

Embodiment C-34. The conjugate of Embodiment C-33, wherein the conjugate is of formula (I′):

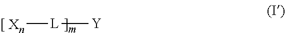

or a prodrug thereof, or a pharmaceutically acceptable salt thereof,
wherein:
n is 1 to 500;
m is 1 to 20;
L is a linker; and
X is an asialoglycoprotein receptor (ASGPR) binding moiety of formula (II):

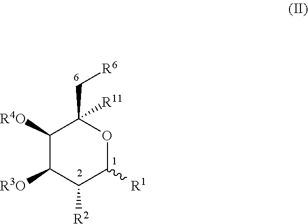

wherein:
R¹ is selected from —Z¹—*, —H, —OH, optionally substituted (C₁-C₆)alkyl, —OCH₃, —OCH₂CH═CH, optionally substituted —S—(C₁-C₆)alkyl, optionally substituted aryl, optionally substituted heteroaryl, optionally substituted —S-aryl, and optionally substituted —S-heteroaryl;
R² is selected from —Z¹—*, —NHCOCH₃, —NHCOCF₃, —NHCOCH₂CF₃, —OH, —NHR, and optionally substituted triazole;
R⁶ is selected from —Z¹—*, —OH, —OR, optionally substituted (C₁-C₆)alkyl, —OC(O)R, —C(O)NHR, —NR^{xx}R^{yy}, optionally substituted aryl, optionally substituted heteroaryl, —NHCOR, and —NRCOR;
each R is independently optionally substituted (C₁-C₆)alkyl, optionally substituted aryl, or optionally substituted heteroaryl;
R^{xx} and R^{yy} are independently H, optionally substituted (C₁-C₆)alkyl, or R^{xx} and R^{yy} can cyclize to form an optionally substituted heterocyclyl;
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, - CONR²¹—, —NR²¹SO₂—, —SO₂NR²¹—, —NR²¹C(═O)NR²¹—, and —NR²¹C(═S)NR²¹—;
Z¹¹— is —O—, —S—, —N(R²¹)—, or —C(R²²)₂; provided that when R¹ is-Z¹—*, and Z¹ is —Z¹¹—, then —Z¹¹— is not —O—;
A¹- and -A²- are optionally substituted heterocyclylene, optionally substituted arylene, or optionally substituted heteroarylene;
each R²¹ is independently selected from H, optionally substituted (C₁-C₆)alkyl, —COR, and optionally substituted heteroaryl; and
each R²² is independently selected from H, halogen, and optionally substituted (C₁-C₆)alkyl.

Embodiment C-35a. The conjugate of Embodiment C-35, wherein The conjugate of C-29, wherein n is 1, 2, or 3 and m is 1-3.

Embodiment C-35. The conjugate of Embodiment C-34, wherein -L-Y comprises:

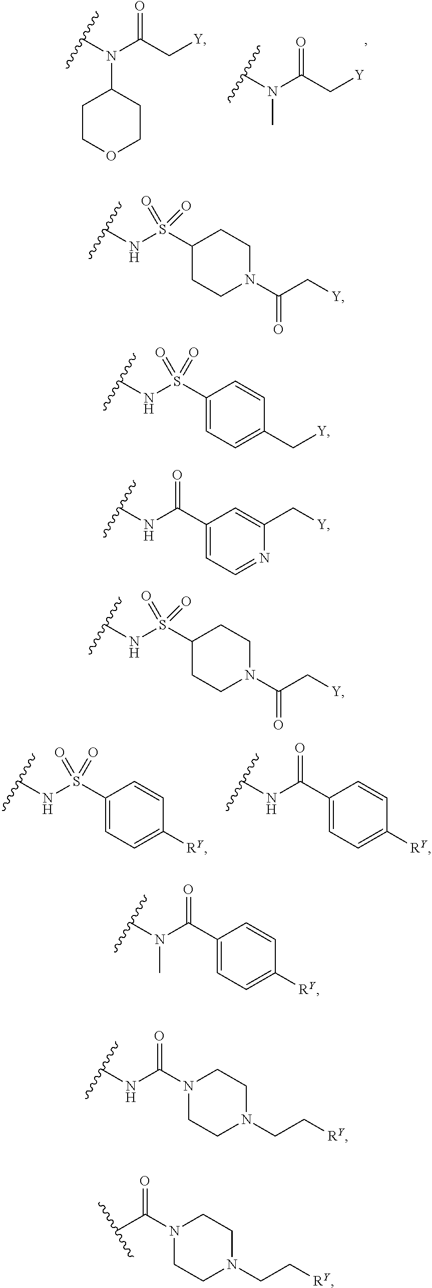

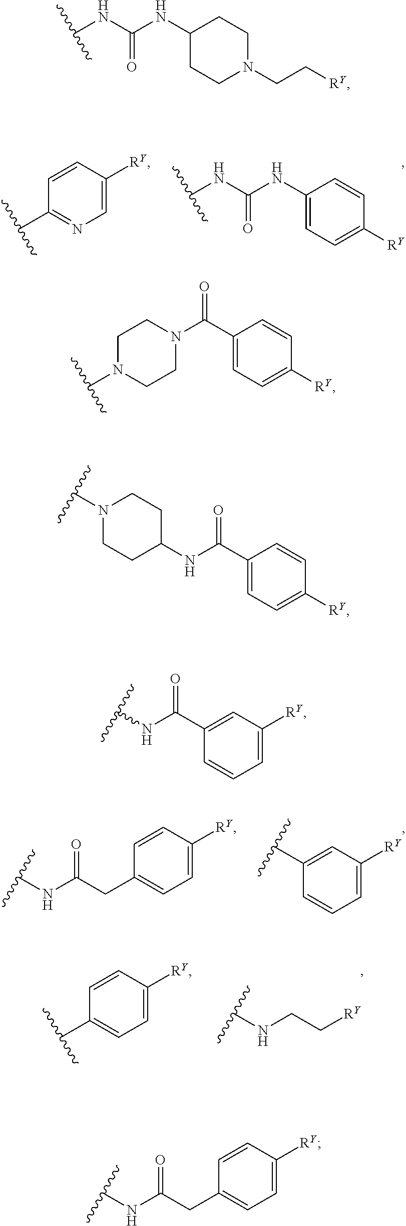

wherein R¹ is

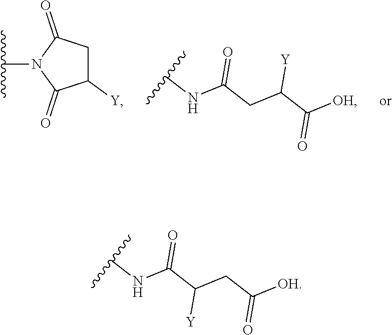

Embodiment C-36. The conjugate of Embodiment C-34 or C-35, wherein X is represented by formula (a-II):

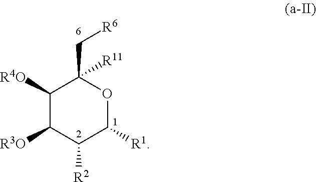

Embodiment C-36a. The conjugate of Embodiment C-34-C-36, wherein R¹ n-propyl and R² is —Z¹—*.

Embodiment C-37. The conjugate of any one of Embodiments C-34 to C-36, wherein R¹ is —Z¹—*, —H, or (C₁-C₆)alkyl.

Embodiment C-38. The conjugate of any one of Embodiments C-34 to C-36, wherein R² is —Z¹—* or —NHCOCH₃.

Embodiment C-39. The conjugate of any one of Embodiments C-34 to C-38, wherein R³ and R are each —H.

Embodiment C-40. The conjugate of Embodiment C-32, wherein X is a moiety that binds to M6PR.

Embodiment C-41. The conjugate of Embodiment C-40, wherein X is of formula (IV):

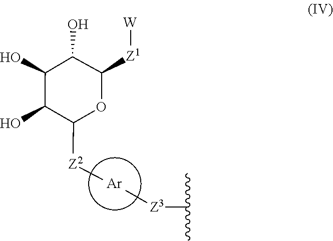

wherein:
W is a non-hydrolyzable hydrophilic head group;
Z¹ is selected from optionally substituted (C₁-C₃)alkylene and optionally substituted ethenylene;
Z² is selected from S, NR²¹ and C(R²²)₂, wherein each R²¹ is independently selected from H, and optionally substituted (C₁-C₆)alkyl, and each R²² is independently selected from H, halogen and optionally substituted (C₁-C₆)alkyl;
each A is independently an optionally substituted aryl or heteroaryl linking moiety; and
each Z³ is independently a linking moiety.

Embodiment C-42. The conjugate of Embodiment C-41, wherein Z² is S.

Embodiment C-43. The conjugate of Embodiment C-41 or C-42, wherein W is phosphonate, thiophosphonate, carboxylic or malonic acid, or a salt thereof.

Embodiment C-44. The conjugate of any one of Embodiments C-45 to C-47, wherein X is:

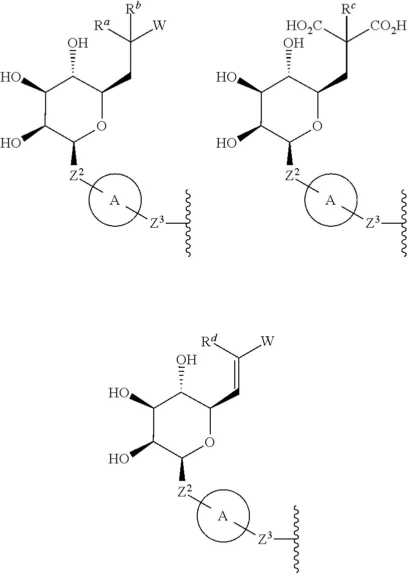

wherein R^{a}, R^{b}, R^{c} and R^{d} are independently H or F.

Embodiment C-45. The conjugate of any one of Embodiments C-41 to C-44, wherein X is:

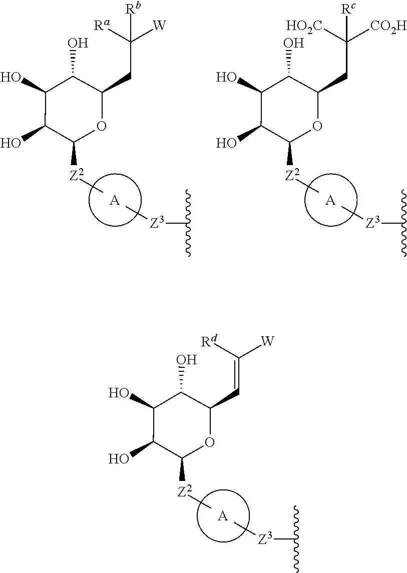

wherein R^{a} R^{b}, R^{c} and R^{d} are independently H or F.

Embodiment C-46. The conjugate of any one of Embodiments C-41 to C-45, wherein A is optionally substituted aryl or optionally substituted heteroaryl, preferably A is independently selected from optionally substituted phenyl, optionally substituted pyridyl, optionally substituted biphenyl, optionally substituted naphthalene, optionally substituted triazole and optionally substituted phenylene-triazole.

Embodiment C-47. The conjugate of any preceding Embodiment, wherein L comprises of 10 to 60 consecutive branched or linear chain atoms.

Embodiment C-48. The conjugate of any preceding Embodiment, wherein L is of formula (IIb′):

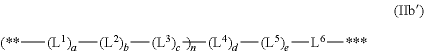

wherein:
n is 1, 2, or 3;
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

Embodiment C-49. The conjugate of Embodiment C-48, wherein each L¹ to L⁵ independently comprises one or more linking moieties independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S-monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50;
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹⁶—, —C₁₋₆-alkylene-NR¹⁶C(S)NR¹⁶—, —O(CH₂)ₚ—, —(OCH₂CH₂)ₚ—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹—; and
each R¹⁶ is independently —H, optionally substituted (C₁-C₆)alkyl, optionally substituted aryl, optionally substituted monocyclic heteroaryl or monocyclic heteroaryl.

Embodiment C-50. The conjugate of Embodiment C-48, wherein each L¹ to L⁵ is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C( )NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50; and

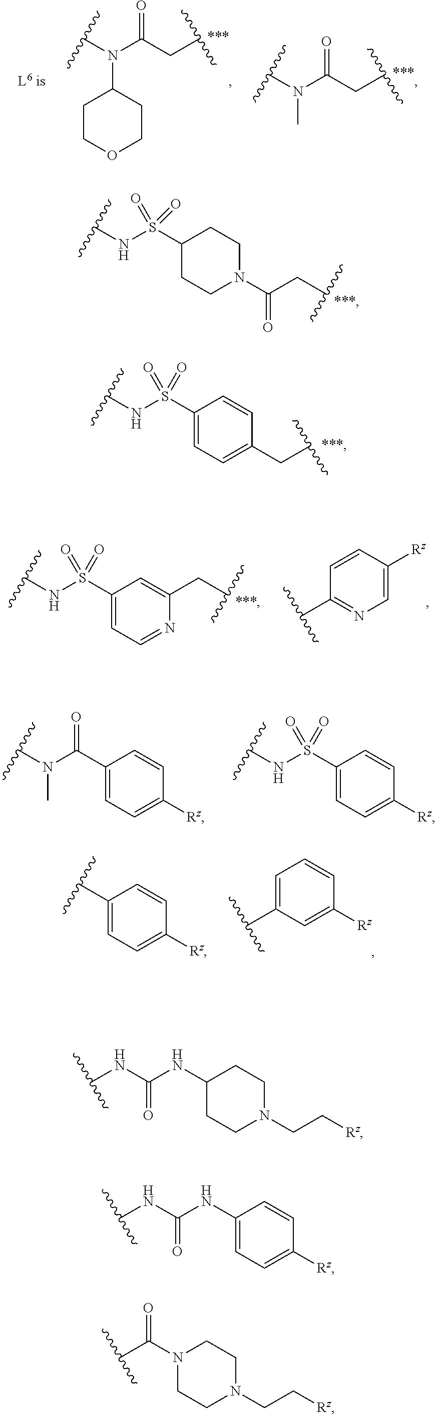

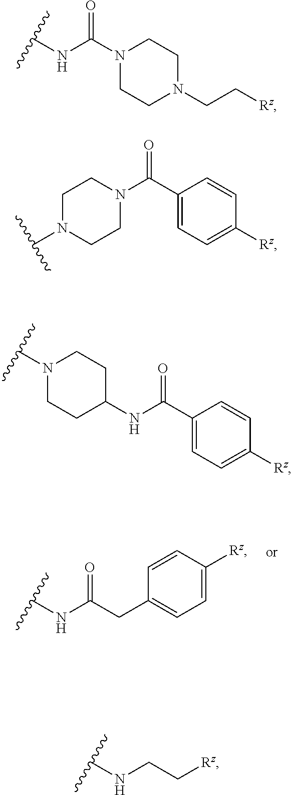

wherein R^{z} is

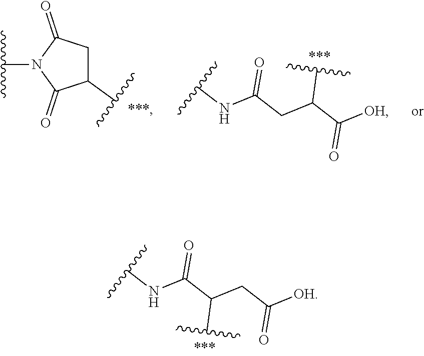 [SMILES: *C1CC(=O)N(*)C1=O ; *NC(=O)CC(*)C(=O)O ; *NC(=O)C(*)CC(=O)O]

Embodiment C-50. A method of degrading a target molecule in a subject in need thereof, comprising administering an effective amount of a conjugate of any preceding Embodiment to the subject.

Embodiment C-51. The method of Embodiment C-50, wherein at least 90% of the target is degraded at four days following the administration.

Embodiment C-52. The method of Embodiment C-50, wherein at least 90% of the target is degraded at seven days following the administration.

Embodiment C-53. The method of Embodiment C-50, wherein the extracellular concentration of the target is substantially maintained for a time period of at least four days, at an amount of at least 90% less than the initial extracellular concentration of the target prior to administering an effective amount of the conjugate.

Embodiment C-54. The method of Embodiment C-50, wherein the time period is seven days or more.

Embodiment C-55. The method of any one of Embodiments C-50 to C-54, wherein a super-stoichiometric target:conjugate ratio is degraded.

Embodiment C-56. The method of Embodiment C-55, wherein the ratio is at least about 5.

Embodiment C-57. The method of any one of Embodiments C-50 to C-56, wherein the target-binding moiety, Y, has a higher binding affinity for the target molecule extracellularly than intraendosomally.

Embodiment C-58. The method of any one of Embodiments C-50 to C-57, wherein the target is IgE.

Embodiment C-59. A method of degrading a target molecule in a subject in need thereof, comprising administering an effective amount of a conjugate that comprises:
a means for binding a lysosomal targeting molecule extracellularly;
a means for binding a target molecule extracellularly;
a means for dissociating from the target molecule intraendosomally; and
wherein the conjugate is externalized from a cell.

Embodiment C-60. The method of Embodiment C-59, wherein the means for binding a lysosomal targeting molecule, remains bound to the lysosomal targeting molecule intraendosomally.

Embodiment C-61. The method of Embodiment C-59 or C-60, wherein the means for binding a target molecule also binds FcRn intraendosomally.

Embodiment C-62. The method of Embodiment C-61, wherein the conjugate dissociates from the lysosomal targeting molecule intraendosomally.

### EXAMPLES

The examples in this section are offered by way of illustration, and not by way of limitation.

### Synthesis of (R)-5-((2-(2-(2 aminoethoxy)ethoxy)ethoxy)methyl)-3-((3S,4R,5R,6R)-4,5-dihydroxy-6-(hydroxymethyl)tetrahydro-2H-pyran-3-yl)oxazolidin-2-one (XB91)

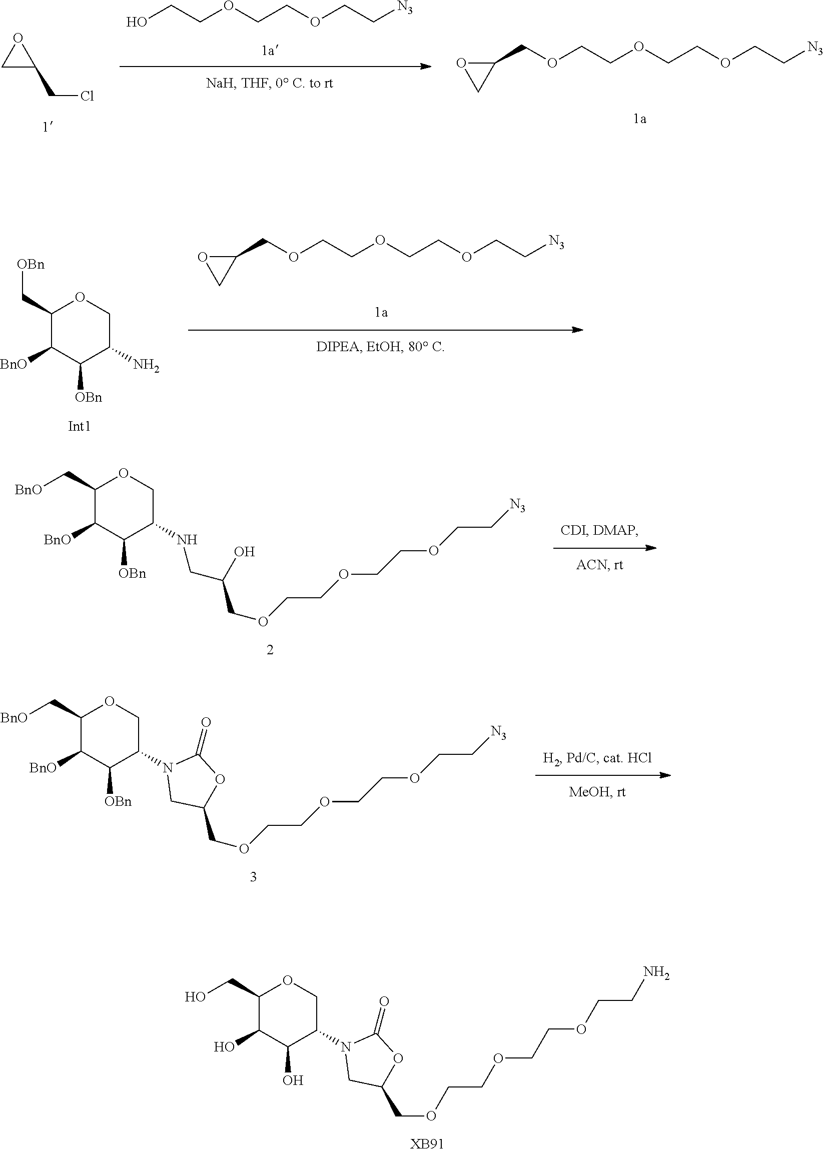

### Synthesis of (R)-2-((2-(2-(2-azidoethoxy)ethoxy)ethoxy)methyl)oxirane (1a)

A solution of 2-(2-(2-azidoethoxy)ethoxy)ethan-1-ol (1a′, 1.0 eq, 0.947 g, 5.4 mmol) in tetrahydrofuran (5 mL) was cooled at 0° C., sodium hydride (60% suspension in mineral oil) (1.1 eq, 0.238 g, 5.94 mmol) was added and reaction mixture was stirred at 0° C. for 30 minutes. Then, a solution of (R)-2-(chloromethyl)oxirane (1′, 1.0 eq, 0.500 g, 5.4 mmol) in tetrahydrofuran (5 mL) was added and reaction mixture was stirred at room temperature for 16 h. After completion, the reaction mixture was poured into ice cold water and extracted with ethyl acetate. The organic layer was dried over anhydrous sodium sulfate, filtered and concentrated to get crude residue which was purified by column chromatography using silica gel (100-200 mesh, 0-100% ethyl acetate in hexane) to afford (R)-2-((2-(2-(2-azidoethoxy)ethoxy)ethoxy)methyl)oxirane (1a) as colorless viscous liquid. Yield: 0.350 g, 28.0%; LCMS m/z 249.1 [M+18]⁺.

### Synthesis of (R)-1-(2-(2-(2-azidoethoxy)ethoxy)ethoxy)-3-(((3S,4R,5R,6R)-4,5-bis(benzyloxy)-6-((benzyloxy)methyl)tetrahydro-2H-pyran-3-yl)amino)propan-2-ol (2)

To a solution of (3S,4R,5R,6R)-4,5-bis(benzyloxy)-6-((benzyloxy)methyl)tetrahydro-2H-pyran-3-amine (Int1, 1.0 eq, 0.450 g, 1.04 mmol) and (R)-2-((2-(2-(2-azidoethoxy)ethoxy)ethoxy)methyl)oxirane (1a, 0.9 eq, 0.216 g, 0.93 mmol) in ethanol (9 mL) N,N-diisopropylethylamine (2.0 eq, 0.38 mL, 2.08 mmol) was added and reaction mixture was heated for at 80° C. for 16 h. After completion, the reaction mixture was concentrated to get crude which was purified by column chromatography using silica gel (100-200 mesh, 0-3% methanol in dichloromethane) to afford (R)-1-(2-(2-(2-azidoethoxy)ethoxy)ethoxy)-3-(((3S,4R,5R,6R)-4,5-bis(benzyloxy)-6-((benzyloxy)methyl)tetrahydro-2H-pyran-3-yl)amino)propan-2-ol (2) as colorless viscous liquid. Yield: 0.420 g, 60.9%; LCMS m/z 665.7 [M+H]⁺.

### Synthesis of (R)-5-((2-(2-(2-azidoethoxy)ethoxy)ethoxy)methyl)-3-((3S,4R,5R,6R)-4,5-bis(benzyloxy)-6-((benzyloxy)methyl)tetrahydro-2H-pyran-3-yl)oxazolidin-2-one (3)

To a solution of (R)-1-(2-(2-(2-azidoethoxy)ethoxy)ethoxy)-3-(((3S,4R,5R,6R)-4,5-bis(benzyloxy)-6-((benzyloxy)methyl)tetrahydro-2H-pyran-3-yl)amino)propan-2-ol (2, 1.0 eq, 0.420 g, 0.63 mmol) in acetonitrile (10 mL), 1,1′-carbonyldiimidazole (CDI) (1.5 eq, 0.154 g, 0.95 mmol) and 4-dimethylaminopyridine (DMAP) (0.1 eq, 0.007 g, 0.063 mmol) were added and the reaction mixture was stirred at room temperature for 16 h. After completion, the reaction mixture was concentrated to get crude which was purified by column chromatography using silica gel (100-200 mesh, 0-50% ethyl acetate in hexane) to afford (R)-5-((2-(2-(2-azidoethoxy)ethoxy)ethoxy)methyl)-3-((3S,4R,5R,6R)-4,5-bis(benzyloxy)-6-((benzyloxy)methyl)tetrahydro-2H-pyran-3-yl)oxazolidin-2-one (3) as colorless viscous liquid. Yield: 0.310 g, 71.0%; LCMS m/z 691.7 [M+H]⁺.

### Synthesis of (R)-5-((2-(2-(2 aminoethoxy)ethoxy)ethoxy)methyl)-3-((3S,4R,5R,6R)-4,5-dihydroxy-6-(hydroxymethyl)tetrahydro-2H-pyran-3-yl)oxazolidin-2-one (XB91)

To a solution of (R)-5-((2-(2-(2-azidoethoxy)ethoxy)ethoxy)methyl)-3-((3S,4R,5R,6R)-4,5-bis(benzyloxy)-6-((benzyloxy)methyl)tetrahydro-2H-pyran-3-yl)oxazolidin-2-one (3, 1.0 eq, 0.310 g, 0.45 mmol) in methanol (6 mL), conc. hydrochloric acid (0.31 mL) and 10% Palladium on carbon (0.310 g) were added and the reaction mixture was stirred at room temperature under hydrogen for 16 h. Reaction was monitored by ELSD. After completion, reaction mixture was filtered through syringe filter and filtrate was concentrated to get crude which was purified by prep HPLC (14-28% acetonitrile in water with 0.1% trifluoroacetic acid) to afford (R)-5-((2-(2-(2 aminoethoxy)ethoxy)ethoxy)methyl)-3-((3S,4R,5R,6R)-4,5-dihydroxy-6-(hydroxymethyl)tetrahydro-2H-pyran-3-yl)oxazolidin-2-one (XB91) as colorless viscous syrup. Yield: 0.064 g, 36.1%; LCMS m/z 395.1 [M+H]⁺; 1H NMR (400 MHz, DMSO-d₆ with D₂O) δ 4.63-4.61 (m, 1H), 3.76-3.72 (m, 2H), 3.62-3.47 (m, 15H), 3.45 (d, J=5.2 Hz, 2H), 3.27-3.22 (m, 3H), 2.94 (t, J=4.8 Hz, 2H).

### Synthesis of (3S,4R,5R,6R)-4,5-bis(benzyloxy)-6-((benzyloxy)methyl)tetrahydro-2H-pyran-3-amine (Int1 in synthesis of XB91)

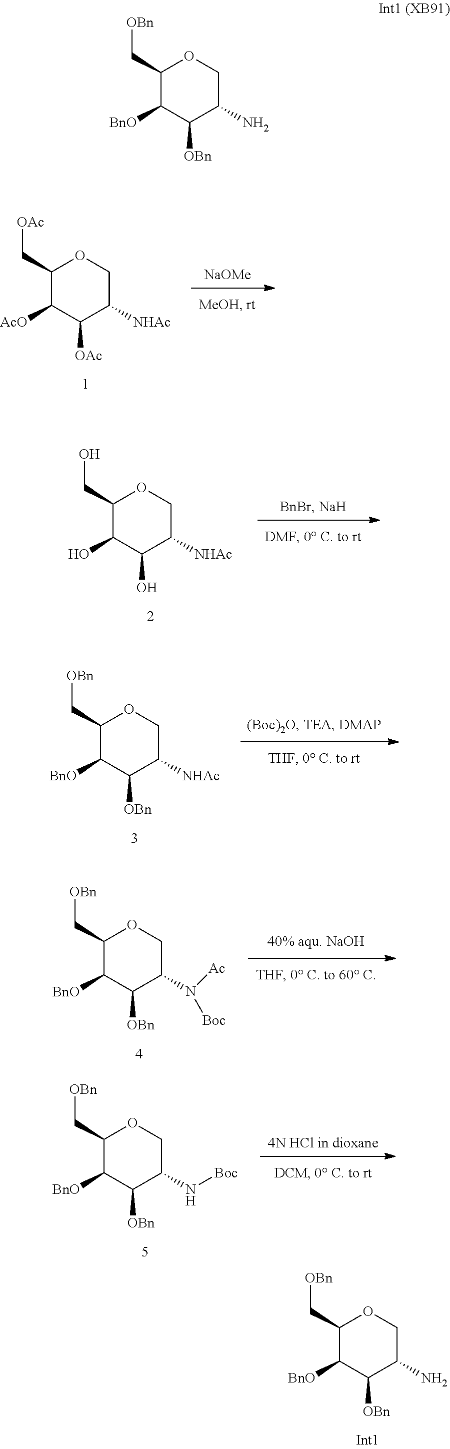

### Synthesis of N-((3S,4R,5R,6R)-4,5-dihydroxy-6-(hydroxymethyl)tetrahydro-2H-pyran-3-yl)acetamide (2)

A solution of (2R,3R,4R,5S)-5-acetamido-2-(acetoxymethyl)tetrahydro-2H-pyran-3,4-diyl diacetate (1, 1.0 eq, 7.8 g, 23.5 mmol) in methanol (78 mL) was cooled at 0° C., sodium methoxide (25% solution in methanol) (0.2 eq, 1.13 mL, 4.71 mmol) was added and reaction mixture was stirred at room temperature for 16 h. After completion, reaction mixture was neutralized with Dowex 50WX8 hydrogen form (200-400 mesh) and filtered through sintered funnel (without celite). The filtrate was concentrated, washed with diethyl ether and dried to afford N-((3S,4R,5R,6R)-4,5-dihydroxy-6-(hydroxymethyl)tetrahydro-2H-pyran-3-yl)acetamide (2) as an off white solid. Yield: 3.1 g, 64.17%; LCMS m/z 206.15 [M+1]⁺.

### Synthesis of N-((3S,4R,5R,6R)-4,5-bis(benzyloxy)-6-((benzyloxy)methyl)tetrahydro-2H-pyran-3-yl)acetamide (3)

A solution of N-((3S,4R,5R,6R)-4,5-dihydroxy-6-(hydroxymethyl)tetrahydro-2H-pyran-3-yl)acetamide (2, 1.0 eq, 1.0 g, 4.87 mmol) in N,N-dimethylformamide (10 mL) was cooled at 0° C., sodium hydride (60% suspension in mineral oil, 4.5 eq, 0.840 g, 21.9 mmol) was added and reaction mixture was stirred at 0° C. for 30 minutes. Then, benzyl bromide (4.5 eq, 2.68 mL, 21.9 mmol) was added and reaction mixture was stirred at room temperature for 16 h. After completion, reaction mixture was poured into ice cold water and extracted with ethyl acetate. The organic layer was dried over anhydrous sodium sulfate, filtered and concentrated to get crude which was purified by column chromatography using silica gel (100-200 mesh) and 0-10% ethyl acetate in hexane to afford N-((3S,4R,5R,6R)-4,5-bis(benzyloxy)-6-((benzyloxy)methyl)tetrahydro-2H-pyran-3-yl)acetamide (3) as a light yellow viscous liquid. Yield: 1.2 g, 51.78%; LCMS m/z 476.10 [M+1]⁺.

### Synthesis of tert-butyl (2-(2-(2-(2-(((2R,3R,4R,5R,6R)-3-acetamido-4,5-dihydroxy-6-(hydroxymethyl)tetrahydro-2H-pyran-2-yl)thio)ethoxy)ethoxy)ethoxy)ethyl)carbamate (4)

A solution of tert-butyl acetyl((3S,4R,5R,6R)-4,5-bis(benzyloxy)-6-((benzyloxy)methyl)tetrahydro-2H-pyran-3-yl)carbamate (3, 1.0 eq, 0.500 g, 1.05 mmol) in tetrahydrofuran (5 mL) was cooled at 0° C., 4-dimethylamino pyridine (0.2 eq, 0.025 g, 0.210 mmol), triethylamine (3.0 eq, 0.44 mL, 3.15 mmol) and boc anhydride (20.0 eq, 4.83 mL, 21.0 mmol) were added and reaction mixture was stirred at room temperature for 48 h. After completion, reaction mixture was concentrated to get crude which was purified by column chromatography using silica gel (100-200 mesh) and 0-50% ethyl acetate in hexane to afford tert-butyl acetyl((3S,4R,5R,6R)-4,5-bis(benzyloxy)-6-((benzyloxy)methyl)tetrahydro-2H-pyran-3-yl)carbamate (4) as a light yellow viscous liquid. Yield: 0.500 g, 82.61%; LCMS m/z 593.15 [M+18]⁺.

### Synthesis of tert-butyl ((3S,4R,5R,6R)-4,5-bis(benzyloxy)-6-((benzyloxy)methyl)tetrahydro-2H-pyran-3-yl)carbamate (5)

A solution of tert-butyl acetyl((3S,4R,5R,6R)-4,5-bis(benzyloxy)-6-((benzyloxy)methyl)tetrahydro-2H-pyran-3-yl)carbamate (4, 1.0 eq, 0.500 g, 0.869 mmol) in tetrahydrofuran (5 mL) was cooled at 0° C., 40% aqueous sodium hydroxide solution (1 mL) was added and reaction mixture was heated at 60° C. for 16 h. After completion, reaction mixture was cooled, water was added and extracted with ethyl acetate The organic layer was dried over anhydrous sodium sulfate, filtered and concentrated to get crude which was purified by column chromatography using silica gel (100-200 mesh) and 0-40% ethyl acetate in hexane to afford tert-butyl ((3S,4R,5R,6R)-4,5-bis(benzyloxy)-6-((benzyloxy)methyl)tetrahydro-2H-pyran-3-yl)carbamate (5) as an off white solid. Yield: 0.340 g, 73.36%; LCMS m/z 534.10 [M+1]⁺.

### Synthesis of (3S,4R,5R,6R)-4,5-bis(benzyloxy)-6-((benzyloxy)methyl)tetrahydro-2H-pyran-3-amine (Int1)

A solution of tert-butyl ((3S,4R,5R,6R)-4,5-bis(benzyloxy)-6-((benzyloxy)methyl)tetrahydro-2H-pyran-3-yl)carbamate (5, 1.0 eq, 0.340 g, 0.637 mmol) in dichloromethane (1.7 mL) was cooled at 0° C., 4N hydrochloric acid in 1,4-dioxane (1.7 mL) was added and reaction mixture was stirred at room temperature for 1 h. After completion, reaction mixture was concentrated, azeotroped with dichloromethane (2-3 times), washed with diethyl ether (2-3 times) and dried to afford (3S,4R,5R,6R)-4,5-bis(benzyloxy)-6-((benzyloxy)methyl)tetrahydro-2H-pyran-3-amine (Int1) as a cream solid. Yield: 0.235 g, 85.08%; LCMS m/z 434.10 [M+1]+; 1H NMR (400 MHz, DMSO-d6) δ 8.11 (bs, 2H), 7.48-7.46 (m, 2H), 7.39-7.26 (m, 11H), 7.23-7.20 (m, 2H), 4.80 (d, J=11.2 Hz, 1H), 4.69 (d, J=11.2 Hz, 1H), 4.58 (d, J=11.2 Hz, 1H), 4.52-4.43 (m, 3H), 4.15-4.14 (m, 1H), 4.03-3.99 (m, 1H), 3.81-3.78 (m, 1H), 3.65 (t, J=6.0 Hz, 1H), 3.52 (d, J=5.6 Hz, 2H), 3.39-3.38 (m, 1H), 3.32-3.29 (m, 1H).

### Synthesis of XB48

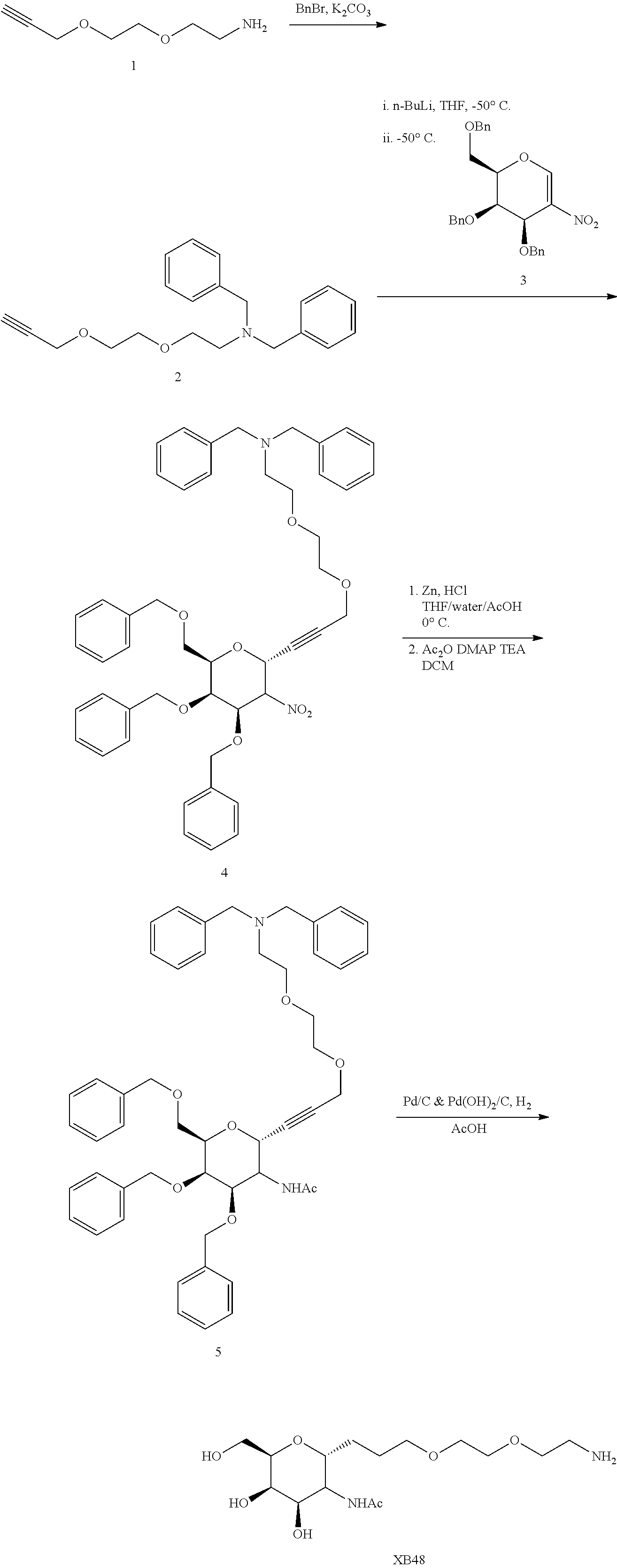

### Synthesis of N,N-dibenzyl-2-(2-((3-((2R,3S,4R,5R,6R)-4,5-bis(benzyloxy)-6-((benzyloxy)methyl)-3-nitrotetrahydro-2H-pyran-2-yl)prop-2-yn-1-yl)oxy)ethoxy)ethan-1-amine (2)

A mixture of 2-[2-(2-propynyloxy)ethoxy]ethylamine (1, 1.00 eq, 9.75 g, 68.1 mmol), anhydrous potassium carbonate (2.32 eq, 21.82 g, 158 mmol) and acetonitrile (306.46 mL) was treated with benzyl bromide (2.10 eq, 17 mL, 143 mmol) then heated to 50° C. for 2 h. The reaction was filtered through celite and the filtrate concentrated under vacuum. The residue was adsorbed to silica then purified by column chromatography (5-100% EtOAc in hexanes) to give N,N-dibenzyl-2-(2-prop-2-ynoxyethoxy)ethanamine 2 as a clear oil. Yield: 18.4 g, 83%. LCMS m/z 324.22 [M+H].

N,N-dibenzyl-2-(2-prop-2-ynoxyethoxy)ethanamine (2, 1.43 eq, 3.70 g, 11.4 mmol) was dissolved in 10 mL toluene then concentrated to dryness and left under high vacuum. Next, (2R,3R,4R)-3,4-dibenzyloxy-2-(benzyloxymethyl)-5-nitro-3,4-dihydro-2H-pyran (3, 1.00 eq, 3.70 g, 8.02 mmol) was dissolved in 10 mL toluene and concentrated under high vacuum. In an oven dried flask, a solution of N,N-dibenzyl-2-(2-prop-2-ynoxyethoxy)ethanamine (2, 1.43 eq, 3.70 g, 11.4 mmol) in anhydrous THF (31.8 mL) under nitrogen via balloon was cooled to −50° C. (dry-ice bath - 1:1 MeOH/water) then treated with the slow addition of butyl lithium, 2.5M in hexanes (1.20 eq, 3.8 mL, 9.62 mmol) and the reaction was stirred @-50° C. for 60 minutes. Next, a solution of (2R,3R,4R)-3,4-dibenzyloxy-2-(benzyloxymethyl)-5-nitro-3,4-dihydro-2H-pyran (3, 1.00 eq, 3.70 g, 8.02 mmol) in dry THF (12.7 mL) was added dropwise over 5 minutes while maintaining −50° C. externally. After 60 minutes, the reaction was quenched with the addition of 7.1M aq. ammonium chloride (32.8 eq, 37 mL, 263 mmol) until a reaction pH of 9-10 was reached, then the cold bath was removed and the slurry was warmed to room temperature. The desired product was extracted with EtOAc (100 mL, 50 mL) and the combined organic layer was dried over Na2SO4, filtered and concentrated under reduced pressure to give crude oil. The oil was adsorbed to silica gel then purified by silica gel chromatography (10% then 20% 2-MeTHF in hexanes) to give: N,N-dibenzyl-2-[2-[3-[(2R,3S,4R,5R,6R)-4,5-dibenzyloxy-6-(benzyloxymethyl)-3-nitro-tetrahydropyran-2-yl]prop-2-ynoxy]ethoxy]ethanamine (4, 2.19 g, 2.79 mmol, 35% yield). LCMS m/z 785.34 [M+H] and N,N-dibenzyl-2-(2-((3-((2S,3S,4R,5R,6R)-4,5-bis(benzyloxy)-6-((benzyloxy)methyl)-3-nitrotetrahydro-2H-pyran-2-yl)prop-2-yn-1-yl)oxy)ethoxy)ethan-1-amine (3.11 g, 3.9 mmol, 49% yield)

A solution of N,N-dibenzyl-2-[2-[3-[(2R,3S,4R,5R,6R)-4,5-dibenzyloxy-6-(benzyloxymethyl)-3-nitro-tetrahydropyran-2-yl]prop-2-ynoxy]ethoxy]ethanamine (4, 1.00 eq, 2.40 g, 3.06 mmol) in THF (182 mL), water (77.6 mL) and acetic acid (46.1 mL) was cooled over ice then treated with zinc dust (18.8 eq, 3.76 g, 57.5 mmol) followed by 12M HCl (44.3 eq, 11 mL, 135 mmol). After 90m, the reaction was filtered then re-cooled in an ice bath before it was treated with 5M aq. sodium hydroxide (350 eq, 214 mL, 1070 mmol) at such a rate as to keep the internal temperature below 24° C. Next, the layers were partitioned then the aqueous layer was washed with DCM (80 mL). The aqueous layer was washed with DCM (50 mL) then the combined organic layer was washed with brine then dried over Na2SO4, filtered, concentrated under reduced pressure and left under high vacuum.

The crude amine from step 3 (2.3 g) was dissolved in DCM (20 mL) before adding triethylamine (6.00 eq, 2.6 mL, 18.3 mmol), 4-(dimethylamino)pyridine (0.050 eq, 18.7 mg, 0.153 mmol) then acetic anhydride (9.80 eq, 2.8 mL, 30.0 mmol). After several hours, the reaction was quenched with water (20 mL) for several minutes. The organic layer was collected, and the aqueous layer was washed with DCM (2×10 mL). The organic layer was dried over Na2SO4, filtered and concentrated in the presence of silica gel for purification by silica gel chromatography (0-15% 2 -MeTHF in DCM) to give N-[(2R,3S,4R,5R,6R)-4,5-dibenzyloxy-6-(benzyloxymethyl)-2-[3-[2-[2-(dibenzylamino)ethoxy]ethoxy]prop-1-ynyl]tetrahydropyran-3-yl]acetamide (5). Yield: 1.86 g, 76%. LCMS m/z 797.5 [M+H].

A mixture of N-[(2R,3S,4R,5R,6R)-4,5-dibenzyloxy-6-(benzyloxymethyl)-2-[3-[2-[2-(dibenzylamino)ethoxy]ethoxy]prop-1-ynyl]tetrahydropyran-3-yl]acetamide (5, 1.00 eq, 2.02 g, 2.53 mmol), and 10% Pd/C w/w (dry basis) (0.040 eq, 0.22 g, 0.101 mmol) in acetic acid (86 mL) under nitrogen was evacuated then back-filled with hydrogen gas via balloon - a process that was repeated 3 times before leaving under an atmosphere of hydrogen. After 1 h, 10% Pd/C w/w (dry basis) (0.200 eq, 1.08 g, 0.507 mmol) and 20% w/w (dry basis) palladium hydroxide (0.300 eq, 1.07 g, 0.760 mmol) were added and the reaction was again placed under hydrogen. After 3 more hours, the reaction was filtered over a pad of celite then the filter cake was rinsed while stirring with methanol (100 mL). Solvents were removed under reduced pressure and the residue was concentrated from toluene then left under high vacuum. The residue was purified by RPHPLC (5-20% ACN in water w/20 mM NH4OH) and fractions lyophilized to give N,N-dibenzyl-2-(2-((3-((2R,3S,4R,5R,6R)-4,5-bis(benzyloxy)-6-((benzyloxy)methyl)-3-nitrotetrahydro-2H-pyran-2-yl)prop-2-yn-1-yl)oxy)ethoxy)ethan-1-amine, XB48, as a white solid. Yield: 562 mg, 63%. LCMS m/z 351.4 [M+H].

### Synthesis of (2R,3R,4R,5S)-5-((4-(3-(2-(2-aminoethoxy)ethoxy)propyl)pyrimidin-2-yl)amino)-2-(hydroxymethyl)tetrahydro-2H-pyran-3,4-diol (XB85)

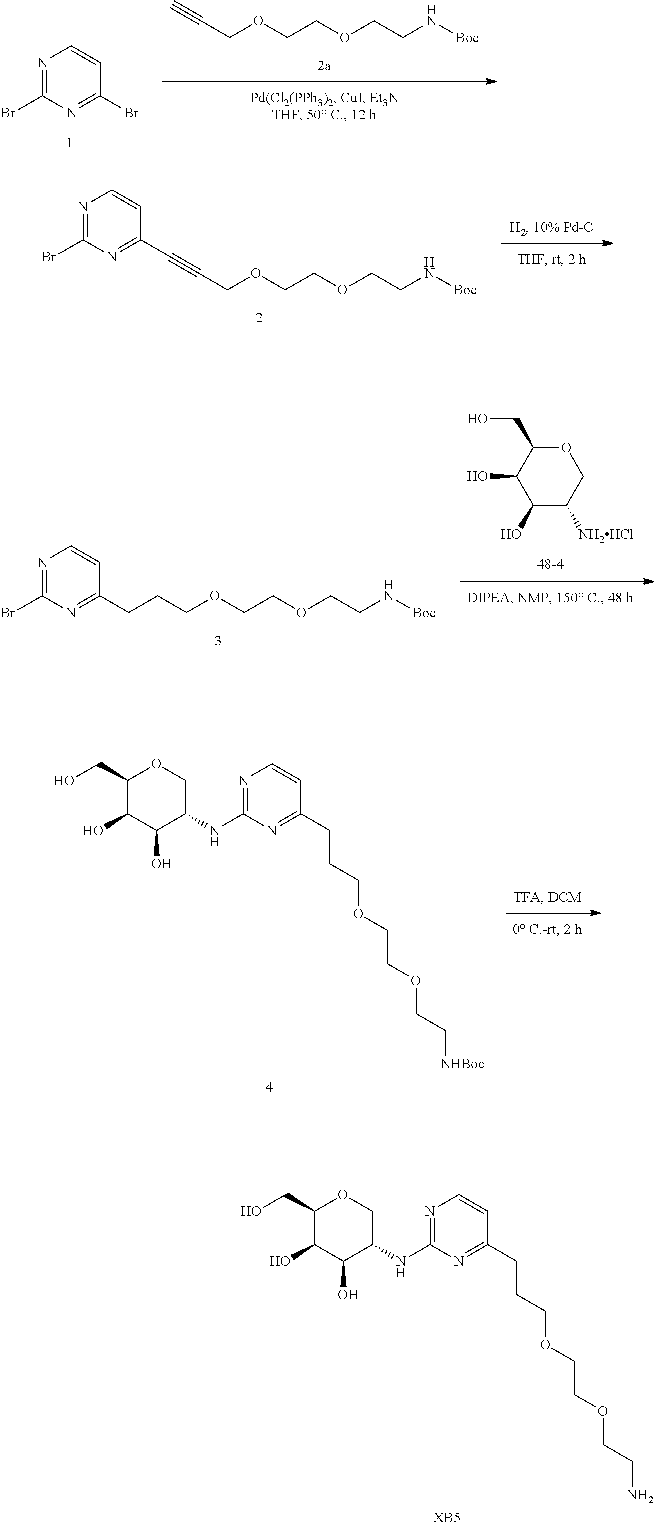

### Synthesis of tert-butyl (2-(2-((3-(2-bromopyrimidin-4-yl)prop-2-yn-1-yl)oxy)ethoxy)ethyl)carbamate (2)

A solution of 2,4-dibromopyrimidine (1, 0.5 g, 1.0 eq., 2.1 mmol) and tert-butyl-2-[2-(2-propynyloxy)ethoxy]ethylaminoformylate (2a, 0.511 g, 1.0 eq., 2.1 mmol) in tetrahydrofuran (5 mL) was purged with N₂ gas for 10 min. To this, triethylamine (0.880 mL, 3 eq., 6.31 mmol), Bis(triphenylphosphine)palladium(II) dichloride (0.0738 g, 0.05 eq., 0.105 mmol) and copper (I) iodide (0.04 g, 0.1 eq., 0.210 mmol) were added, and the reaction mixture was purged with N₂ for another 30 min. Then the resultant reaction mixture was stirred at 50° C. for 12 h. After completion, the reaction mixture was filtered using sintered funnel, and the filtrate was concentrated to obtained crude, which was purified by silica gel column chromatography eluting with 30% ethyl acetate-heptane to afford tert-butyl (2-(2-((3-(2-bromopyrimidin-4-yl)prop-2-yn-1-yl)oxy)ethoxy)ethyl)carbamate (2) as colorless viscous liquid. Yield: 0.45 g, 53.40%; LCMS: m/z 400.0 [M+H].

### Synthesis of tert-butyl (2-(2-(3-(2-bromopyrimidin-4-yl)propoxy)ethoxy)ethyl)carbamate (3)

To a solution of tert-butyl (2-(2-((3-(2-bromopyrimidin-4-yl)prop-2-yn-1-yl)oxy)ethoxy)ethyl)carbamate (2, 0.28 g, 0.7 mmol) in tetrahydrofuran (6.0 mL), 10% Pd/C (0.28 g) was added and the reaction mixture was stirred at room temperature under hydrogen gas balloon pressure for 4 h. After completion, reaction mixture was filtered through syringe filter and washed with methanol. The filtrate was concentrated and dried to obtained crude. Crude was purified by silica gel chromatography using 10% methanol in dichloromethane to afford tert-butyl (2-(2-(3-(2-bromopyrimidin-4-yl)propoxy)ethoxy)ethyl)carbamate (3) as colourless liquid. Yield: 0.11 g, 38.5%; LCMS: m/z 403.9 [M+H].

### Synthesis of tert-butyl (2-(2-(3-(2-(((3S,4R,5R,6R)-4,5-dihydroxy-6-(hydroxymethyl)tetrahydro-2H-pyran-3-yl)amino)pyrimidin-4-yl)propoxy)ethoxy)ethyl)carbamate (4)

To a stirred solution of tert-butyl (2-(2-(3-(2-bromopyrimidin-4-yl)propoxy)ethoxy)ethyl)carbamate (3, 0.3 g, 1.0 eq., 742 μmol) and (2R,3R,4R,5S)-5-amino-2-(hydroxymethyl)tetrahydro-2H-pyran-3,4-diol hydrochloride salt (48-4, 0.207 g, 1.4 eq., 1.04 mmol) in N-Methyl-2-pyrrolidone (3 mL), N,N-Diisopropylethylamine (1.29 mL, 10 eq., 7.42 mmol) was added, and the resulting reaction mixture was allowed to stirred at 150° C. for 48 h. After completion, volatiles were removed under reduced pressure to obtain the crude which was purified by silica gel column chromatography using 5-20% methanol in dichloromethane to afford tert-butyl (2-(2-(3-(2-(((3S,4R,5R,6R)-4,5-dihydroxy-6-(hydroxymethyl)tetrahydro-2H-pyran-3-yl)amino)pyrimidin-4-yl)propoxy)ethoxy)ethyl)carbamate (4) as viscous oil. Yield: 0.17 g, 44.73%; LCMS: m/z 487.0 [M+H].

### Synthesis of (2R,3R,4R,5S)-5-((4-(3-(2-(2-aminoethoxy)ethoxy)propyl)pyrimidin-2-yl)amino)-2-(hydroxymethyl)tetrahydro-2H-pyran-3,4-diol (XB85)

To a stirred solution of tert-butyl (2-(2-(3-(2-(((3S,4R,5R,6R)-4,5-dihydroxy-6-(hydroxymethyl)tetrahydro-2H-pyran-3-yl)amino)pyrimidin-4-yl)propoxy)ethoxy)ethyl)carbamate (4, 0.119 g, 1.0 eq., 0.245 mmol) in dichloromethane (3 mL) was added trifluoroacetic acid (0.8 mL) at 0° C. The reaction mixture was stirred at room temperature for 2 h. After completion, the reaction mixture was concentrated under vacuum to give crude which was purified by RP prep-HPLC (20-30% acetonitrile in water with 0.1% trifluoroacetic acid). Fractions containing the desired product were combined and lyophilized to dryness to afford (2R,3R,4R,5S)-5-((4-(3-(2-(2-aminoethoxy)ethoxy)propyl)pyrimidin-2-yl)amino)-2-(hydroxymethyl)tetrahydro-2H-pyran-3,4-diol (XB85) as white solid. Yield: 0.064 g, 67.71%; LCMS m/z 387.15 [M+H]; H NMR (400 MHz, DMSO-d₆D₂O exchange): δ 8.12 (d, J=5.20 Hz, 1H), 6.62 (d, J=5.60 Hz, 1H), 4.08 (m, 1H), 3.87-3.83 (m, 1H), 3.73 (d, J=3.20 Hz, 1H), 3.57-3.47 (m, 9H), 3.41 (t, J=6.40 Hz, 2H), 3.29 (t, J=6.00 Hz, 1H), 3.02 (t, J=10.80 Hz, 1H), 2.94 (t, J=5.20 Hz, 2H), 2.60 (t, J=7.20 Hz, 2H), 1.88-1.81 (m, 2H).

### Synthesis of N-((2R,3R,4R,5R,6R)-2-ethynyl-4,5-dihydroxy-6-(hydroxymethyl)tetrahydro-2H-pyran-3-yl)acetamide (XB44A)

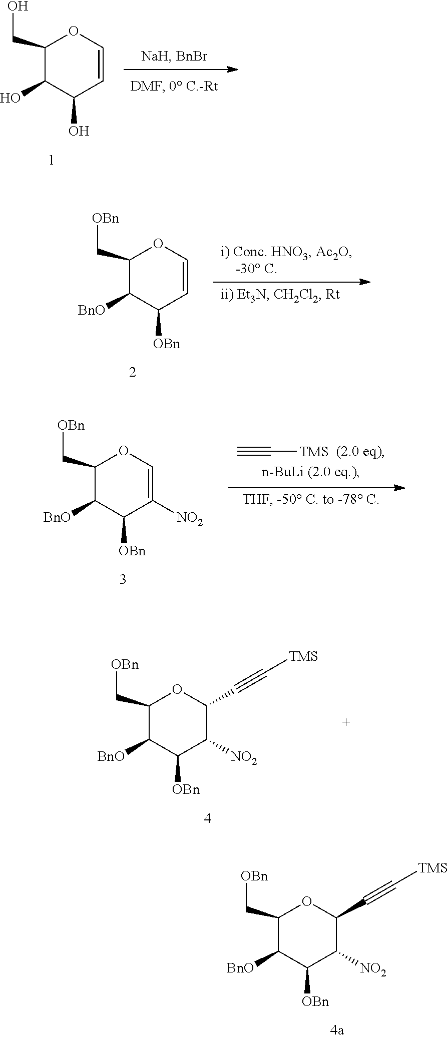

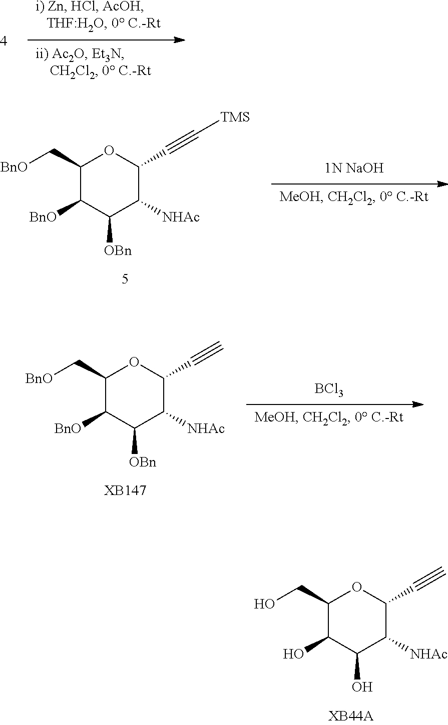

### Synthesis of (2R,3R,4R)-3,4-bis(benzyloxy)-2-((benzyloxy)methyl)-3,4-dihydro-2H-pyran (2)

A solution of (2R,3R,4R)-2-(hydroxymethyl)-3,4-dihydro-2H-pyran-3,4-diol (1, 1.0 eq., 40 g, 274 mmol) in N,N-dimethylformamide (800 mL) was cooled to 0° C. and sodium hydride (3.6 eq., 39.4 g, 985 mmol) was added portion wise. The suspension was stirred at the same temperature for 30 min. Then, benzyl bromide (4.5 eq., 146 mL, 1.23 mmol) was added dropwise to the solution and stirred the reaction mixture at room temperature for 16 h. After completion, the reaction mixture was quenched with ice-cold water and extracted with ethyl acetate (3×400 mL). The organic layer was washed with brine solution and dried over anhydrous sodium sulfate. The organic layer was evaporated under reduced pressure to obtain crude. The crude was purified by column chromatography using silica gel (100-200 mesh) and 10% ethyl acetate in hexane to afford (2R,3R,4R)-3,4-bis(benzyloxy)-2-((benzyloxy)methyl)-3,4-dihydro-2H-pyran (2) as an off white solid. Yield: 80.0 g, 71%. LCMS m/z, [M+H]=417.20.

### Synthesis of (2R,3R,4R)-3,4-bis(benzyloxy)-2-((benzyloxy)methyl)-5-nitro-3,4-dihydro-2H-pyran (3)

Concentrated nitric acid (5.0 eq., 16.2 mL, 360 mmol) was added dropwise to acetic anhydride (300 mL) at 0° C. under argon. When the addition was completed, the temperature was further decreased to −30° C. using acetonitrile-dry ice bath. A solution of (2R,3R,4R)-3,4-bis(benzyloxy)-2-((benzyloxy)methyl)-3,4-dihydro-2H-pyran (2, 1 eq., 30.0 g. 72.0 mmol) in acetic anhydride (100 mL) was added slowly to the mixture. The reaction was stirred at 0° C. until the starting material disappeared. The resulting mixture was then poured into ice-water (220 mL) and brine (40 mL), and the aqueous layer was extracted by dichloromethane (400 mL×3). The combined organic solvent was dried over anhydrous sodium sulfate and evaporated. After removal of the solvent, the residue was dissolved in dichloromethane (300 mL), Et₃N (1.0 eq., 10.1 mL, 72.0 mmol) was added and the mixture was stirred at room temperature for 3 h. After completion of the reaction, the solvent was removed under reduced pressure, and the residue was purified by column chromatography using silica gel (100-200 mesh) and 10-15% ethyl acetate in hexane to afford (2R,3R,4R)-3,4-bis(benzyloxy)-2-((benzyloxy)methyl)-5-nitro-3,4-dihydro-2H-pyran (3) as pale yellow viscous liquid. Yield: 20.0 g, 60.0%. LC-MS m/z, 25 [M+H]=462.12.

### Synthesis of (((2R,3S,4R,5R,6R)-4,5-bis(benzyloxy)-6-((benzyloxy)methyl)-3-nitrotetrahydro-2H-pyran-2-yl)ethynyl)trimethylsilane (4) and (((2S,3S,4R,5R,6R)-4,5-bis(benzyloxy)-6-((benzyloxy)methyl)-3-nitrotetrahydro-2H-pyran-2-yl)ethynyl)trimethylsilane (4a)

To a solution of ethynyltrimethylsilane (2.0 eq., 12.2 mL, 86.7 mmol) in freshly dried tetrahydrofuran (300 mL) at −50° C., n-butyl lithium (2.5 M in hexane) (2.0 eq., 34.7 mL, 86.7 mmol) was added dropwise and continue the stirring at the same temperature for 2 h. After cooling down the temperature to −78° C., (2R,3R,4R)-3,4-bis(benzyloxy)-2-((benzyloxy)methyl)-5-nitro-3,4-dihydro-2H-pyran (3, 1.0 eq, 20 g, 43.3 mmol) in 100 mL THF solution was added dropwise to the reaction mixture and continue the stirring at same temperature for 3 h. A saturated aqueous ammonium chloride solution was then added and the solution was allowed to reach room temperature and was extracted with ethyl acetate (200 mL×3 times). The organic layers were washed with brine, dried over anhydrous Na2SO4, concentrated under reduced pressure. The crude product was purified by column chromatography using silica gel (100-200 mesh) and 5 to 10% ethyl acetate in hexane to give separate diastereomers as (((2R,3S,4R,5R,6R)-4,5-bis(benzyloxy)-6-((benzyloxy)methyl)-3-nitrotetrahydro-2H-pyran-2-yl)ethynyl)trimethylsilane (4) as white solid Yield: 4.7 g, 19.3% and (((2S,3S,4R,5R,6R)-4,5-bis(benzyloxy)-6-((benzyloxy)methyl)-3-nitrotetrahydro-2H-pyran-2-yl)ethynyl)trimethylsilane (4a) as white solid. Yield: 5.2 g, 21.4%. LC-MS m/z [M+18]=641.90

(4) ¹H NMR (400 MHz, CDCl3) δ 7.37-7.22 (m, 15H), 5.22 (d, J 6 Hz, 1H), 5.13-5.09 (m, 1H), 4.84 (d, J 11.2 Hz, 1H), 4.80-4.72 (m, 2H), 4.69-4.35 (m, 4H), 4.15 (t, J 6.4 Hz, 1H), 4.03-4.04 (m, 1H), 3.58-3.53 (m, 2H), 0.19 (s, 9H).

(4a) ¹H NMR (400 MHz, CDCl3) δ 7.37-7.28 (m, 15H), 4.99 (t, J 6 Hz, 1H), 4.84 (d, J 11.2 Hz, 1H), 4.76-4.43 (m, 6H), 4.12-4.03 (m, 2H), 3.67-3.59 (m, 3H), 0.16 (s, 9H).

### Synthesis of N-((2R,3S,4R,5R,6R)-4,5-bis(benzyloxy)-6-((benzyloxy)methyl)-2-((trimethylsilyl)ethynyl)tetrahydro-2H-pyran-3-yl)acetamide (5)

To a solution of (((2R,3S,4R,5R,6R)-4,5-bis(benzyloxy)-6-((benzyloxy)methyl)-3-nitrotetrahydro-2H-pyran-2-yl)ethynyl)trimethylsilane (4, 1.0 eq., 4.7 g, 8.4 mmol) in tetrahydrofuran (20 mL) and water (6 mL) at 0° C. were successively added concentrated hydrochloric acid (10 mL), acetic acid (30 mL) and zinc (19.0 eq., 10.4 g, 160 mmol). The reaction mixture was stirred for 1.5 h at 0° C. After completion, the reaction was filtered over celite bed. The filtrate was extracted with dichloromethane, the organic layer was washed with water, a saturated bicarbonate solution (3 times), brine, and then dried over sodium sulfate.

Solvents were evaporated and the residue was taken up in dry dichloromethane (30 mL), triethylamine (6.5 eq., 7.67 mL, 54.6 mmol), 4-(dimethylamino)pyridin-1-ium (0.5 eq., 0.513 g, 4.2 mmol) and acetyl acetate (25.0 eq., 19.8 mL, 210 mmol) were successively added and the reaction mixture was stirred for 2 h at room temperature. Water was added and the organic layer was washed with brine, dried over sodium sulfate and concentrated. The crude product was purified by column chromatography using silica gel (100-200 mesh) and 0 to 40% ethyl acetate in hexane to afford N-((2R,3S,4R,5R,6R)-4,5-bis(benzyloxy)-6-((benzyloxy)methyl)-2-((trimethylsilyl)ethynyl)tetrahydro-2H-pyran-3-yl)acetamide (5) as a white solid. Yield: 3.7 g, 77.0%. LC-MS m/z [M+H]=572.15.

### Synthesis of N-((2R,3S,4R,5R,6R)-4,5-bis(benzyloxy)-6-((benzyloxy)methyl)-2-ethynyltetrahydro-2H-pyran-3-yl)acetamide (XB147)

To a solution of N-((2R,3S,4R,5R,6R)-4,5-bis(benzyloxy)-6-((benzyloxy)methyl)-2-((trimethylsilyl)ethynyl)tetrahydro-2H-pyran-3-yl)acetamide (5, 1.0 eq., 3.7 g, 6.47 mmol) in methanol (30 mL) and dichloromethane (6 mL) was added an aqueous sodium hydroxide solution (1N, 16 mL). The reaction mixture was stirred at room temperature for 2 h. Reaction was monitored by TLC. After completion, reaction mixture was neutralized by aqueous 1N hydrochloric acid solution. The volatile solvents were evaporated under reduced pressure and the dichloromethane layer was washed with water. The organic layer was dried over sodium sulfate, filtered and concentrated to obtain crude. The crude product was purified by column chromatography using silica gel (100-200 mesh) and 2-3% methanol in dichloromethane to afford N-((2R,3S,4R,5R,6R)-4,5-bis(benzyloxy)-6-((benzyloxy)methyl)-2-ethynyltetrahydro-2H-pyran-3-yl)acetamide (XB147) as a white solid Yield: 3.0 g, 92.8%. LC-MS, m/z, [M+H]=500.24; 1H NMR (400 MHz, DMSO-d6) δ 8.05 (d, J 7.6 Hz, 1H), 7.40-7.20 (t, J 6.0 Hz, 15H), 4.80-4.62 (m, 4H), 4.50-4.40 (m, 3H), 4.40-4.00 (m, 3H), 3.80-3.77 (m, 1H), 3.76-3.40 (m, 3H), 1.86 (s, 3H).

### Synthesis of N-((2R,3R,4R,5R,6R)-2-ethynyl-4,5-dihydroxy-6-(hydroxymethyl)tetrahydro-2H-pyran-3-yl)acetamide (XB44A)

To a mixture of N-[(2R,3S,4R,5R,6R)-4,5-dibenzyloxy-6-(benzyloxymethyl)-2-ethynyl-tetrahydropyran-3-yl]acetamide (1.00 eq, 55.4 mg, 0.111 mmol) in DCM (2.9 mL) at - 10° C. was added boron trichloride solution in DCM (10.5 eq, 1.2 mL, 1.16 mmol) dropwise. The mixture was stirred at rt for 45 minutes and cooled to 0° C. The reaction was quenched with sat. NaHCO₃. The mixture was concentrated to remove organic solvent. The aqueous solution was purified by prep. HPLC (2-10% MeCN/water with 0.1% TFA) to give XB44A as 25 mg of a white solid (98% yield). LC-MS m/z [M+H]=230.2

### Synthesis of N-((2S,3R,4R,5R,6R)-2-(3-((2-(2-aminoethoxy)ethoxy)methyl)isoxazol-5-yl)-4,5-dihydroxy-6-(hydroxymethyl)tetrahydro-2H-pyran-3-yl)acetamide (XB86)

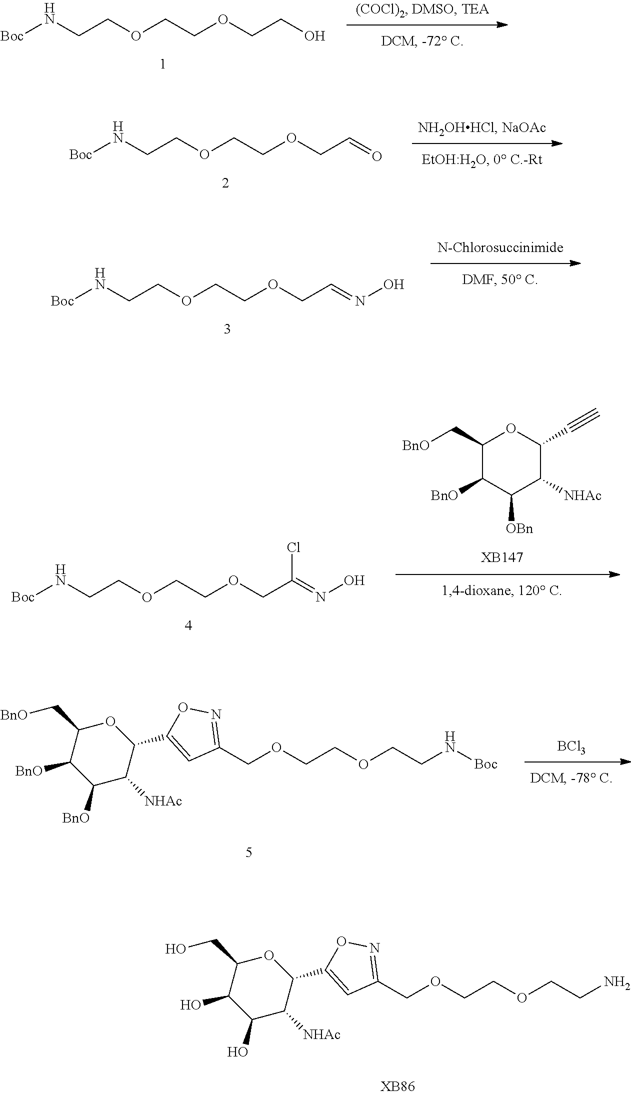

### Synthesis of tert-butyl (2-(2-(2-oxoethoxy)ethoxy)ethyl)carbamate (2)

To a stirred solution of oxalic dichloride (1.38 mL, 2 eq., 16 mmol) in dichloromethane (20 mL) at −78° C. was added dropwise a solution of dimethyl sulfoxide (1.28 mL, 2.25 eq, 18.1 mmol). After being stirred at −78° C. for 20 minutes, a solution of tert-butyl-2-[2-(2-hydroxyethoxy)ethoxy]ethylaminoformylate (1, 2 g, 1 eq., 8.02 mmol) in dichloromethane (20 mL) was added to the mixture. The reaction mixture was further stirred at −78° C. for 90 minutes, followed by addition of triethylamine (9.02 mL, 8.0 eq, 64.2 mmol). The resulting mixture was allowed to reach at room temperature over 1 h. The reaction mixture was diluted with dichloromethane and washed with water followed by brine solution. The organic layer was dried over anhydrous sodium sulphate, filtered, and concentrated under reduced pressure to afford tert-butyl (2-(2-(2-oxoethoxy)ethoxy)ethyl)carbamate (1.9 g, crude) as sticky liquid. The crude was forwarded as such for next step.

### Synthesis of tert-butyl (E)-(2-(2-(2-(hydroxyimino)ethoxy)ethoxy)ethyl)carbamate (3)

To a stirred solution of tert-butyl-2-[2-(formylmethoxy)ethoxy]ethylaminoformylate (2, 1.98 g, 1.0 eq., 8.01 mmol) in ethanol (12 mL), sodium acetate (0.985 g, 1.5 eq., 12 mmol) was added. After being stirred at 0° C., solution of hydroxylamine hydrochloride (0.835 g, 1.5 eq., 12 mmol) in water (4 mL) was added dropwise over 10 min. The reaction mixture was further stirred at room temperature for 12h, After completion (monitored by LCMS), the reaction mixture was diluted with ice cold water and extracted with dichloromethane (3×30 mL). The combined organic phases were dried over anhydrous sodium sulfate, filtered and concentrated under reduced pressure to afford crude residue. The crude was purified by silica gel flash column chromatography using 20% ethyl acetate in hexane as eluent to afford tert-butyl (E)-(2-(2-(2-(hydroxyimino)ethoxy)ethoxy)ethyl)carbamate as a yellowish liquid. Yield: 1.75 g, 83.3%. LCMS: m/z 263.25 [M+H].

### Synthesis of tert-butyl (Z)-(2-(2-(2-chloro-2-(hydroxyimino)ethoxy)ethoxy)ethyl)carbamate (4)

To a stirred solution of tert-butyl (E)-(2-(2-(2-(hydroxyimino)ethoxy)ethoxy)ethyl)carbamate (3, 1.5 g, 1 eq., 5.72 mmol) in N, N-dimethylformaide (10 ml), was added n-chlorosuccinimide (0.764 g, 1 eq., 5.72 mmol) in one portion and stirred at 40° C. for 2h. Reaction was monitored by TLC. After completion of reaction, reaction mixture was diluted with ice cold water and extracted with ethyl acetate (3×20 mL). Then, organic layer was dried over anhydrous sodium sulfate, filtered and concentrated under reduced pressure to afford crude tert-butyl (Z)-(2-(2-(2-chloro-2-(hydroxyimino)ethoxy)ethoxy)ethyl)carbamate (4, 1.6 g) which was used for the next step as such.

### Synthesis of tert-butyl (2-(2-((5-((2S,3R,4R,5R,6R)-3-acetamido-4,5-bis(benzyloxy)-6-((benzyloxy)methyl)tetrahydro-2H-pyran-2-yl)isoxazol-3-yl)methoxy)ethoxy)ethyl)carbamate (5)

Compound N-((2R,3S,4R,5R,6R)-4,5-bis(benzyloxy)-6-((benzyloxy)methyl)-2-ethynyltetrahydro-2H-pyran-3-yl)acetamide (XB147, 0.27 g, 1 eq., 0.54 mmol) and tert-butyl (Z)-(2-(2-(2-chloro-2-(hydroxyimino)ethoxy)ethoxy)ethyl)carbamate (4, 1.6 g, 5 eq, 2.7 mmol) were taken in 1,4-dioxane (10 mL) and reaction mixture was stirred under 120° C. for 3 days. Progress of reaction was monitored by LCMS. After completion, reaction mixture was concentrated under reduced pressure to afford crude which was purified by silica gel flash column chromatography using 70-80% ethyl acetate-heptane as eluent to afford tert-butyl (2-(2-((5-((2S,3R,4R,5R,6R)-3-acetamido-4,5-bis(benzyloxy)-6-((benzyloxy)methyl)tetrahydro-2H-pyran-2-yl)isoxazol-3-yl)methoxy)ethoxy)ethyl)carbamate (5) as yellowish sticky liquid. Yield: 0.27 g, 65.7%. LCMS: m/z 760.00 [M+H].

### Synthesis of N-((2S,3R,4R,5R,6R)-2-(3-((2-(2-aminoethoxy)ethoxy)methyl)isoxazol-5-yl)-4,5-dihydroxy-6-(hydroxymethyl)tetrahydro-2H-pyran-3-yl)acetamide (XB86)

To a stirred solution of tert-butyl (2-(2-((5-((2S,3R,4R,5R,6R)-3-acetamido-4,5-bis(benzyloxy)-6-((benzyloxy)methyl)tetrahydro-2H-pyran-2-yl)isoxazol-3-yl)methoxy)ethoxy)ethyl)carbamate (5, 0.27 g, 1.0 eq., 0.93 mmol) in dry dichloromethane (10 mL), trichloroborane (4.34 ml, 12.0 eq., 4.34 mmol 1M solution in dichloromethane) was added dropwise and stirred at −78° C. for 3 h. After completion (monitored by LCMS), reaction mixture was quenched with methanol and concentrated under reduced pressure to afford crude which was purified by RP prep-HPLC (40% acetonitrile in water with 0.1% TFA) to afford N-((2S,3R,4R,5R,6R)-2-(3-((2-(2 -aminoethoxy)ethoxy)methyl)isoxazol-5-yl)-4,5-dihydroxy-6-(hydroxymethyl)tetrahydro-2H-pyran-3-yl)acetamide (XB86) as colorless foamy solid. Yield: 0.072 g, 51%. LCMS: m/z 390.00 [M+H]. ¹H NMR (400 MHz, DMSO-d₆ with D₂O): δ 6.53 (s, 1H), 5.24 (d, J=6.00 Hz, 1H), 4.54 (s, 2H), 4.24-4.20 (m, 1H), 3.83-3.80 (m, 2H), 3.59-3.55 (m, 7H), 3.49 (d, J=6.00 Hz, 2H), 2.94 (t, J=5.20 Hz, 2H), 1.73 (s, 3H).

### Synthesis of (2R,3R,4R,5S)-5-((4-((2-(2-(2-aminoethoxy)ethoxy)ethoxy)methyl)thiazol-2-yl)amino)-2-(hydroxymethyl)tetrahydro-2H-pyran-3,4-diol (XB82)

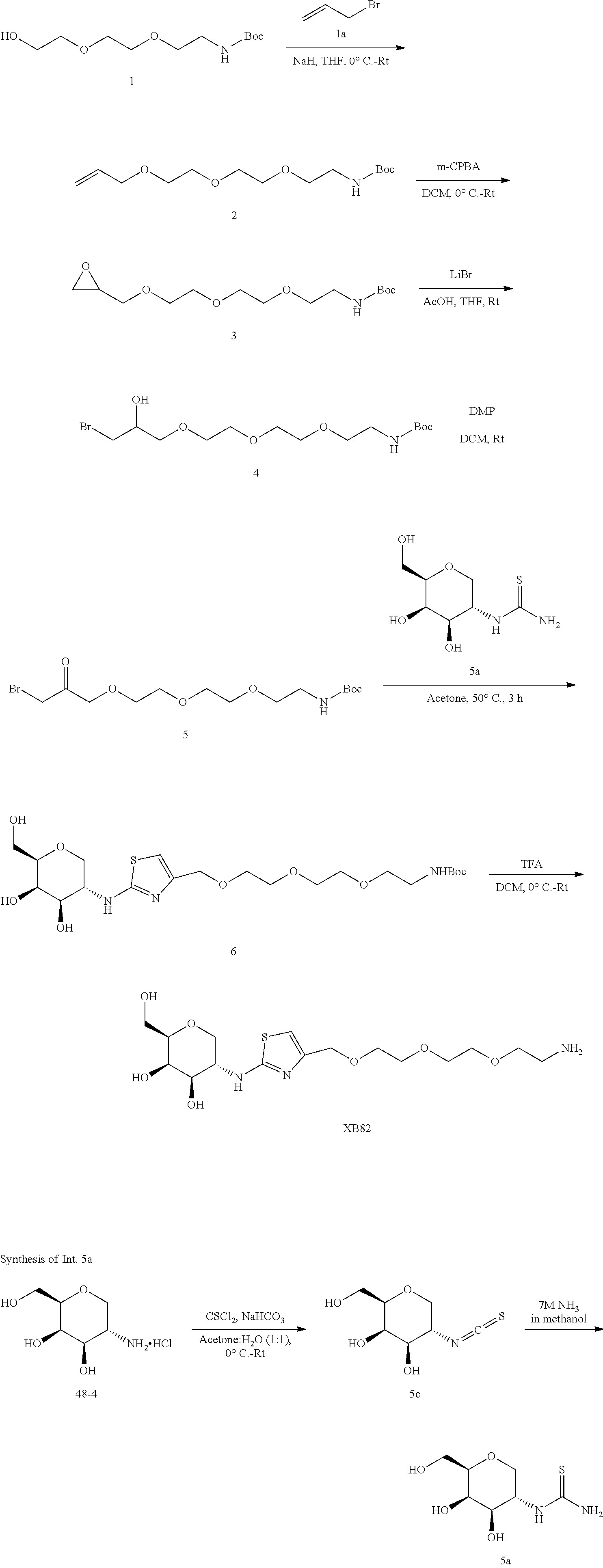

### Synthesis of (2R,3R,4R,5S)-2-(hydroxymethyl)-5-isothiocyanatotetrahydro-2H-pyran-3,4-diol (5c)

To a suspension of (2R,3R,4R,5S)-5-amino-2-(hydroxymethyl)tetrahydro-2H-pyran-3,4-diol hydrochloride (48-4, 0.180 g, 0.902 mmol) in water (1.6 mL) and acetone (1.6 mL) were sequentially added sodium hydrogen carbonate (0.227 g, 3 eq., 2.7 mmol) and thiophosgene (0.082 mL, 1.2 eq., 1.08 mmol). The mixture was stirred at room temperature for 2 h. Progress of the reaction was monitored by TLC and LCMS. After completion, reaction mixture was concentrated to dryness. The residue was co-evaporated with toluene (2×5 mL) and purified by flash column chromatography using 0-4% methanol/ethyl acetate as eluting solvent to afford (2R,3R,4R,5S)-2-(hydroxymethyl)-5-isothiocyanatotetrahydro-2H-pyran-3,4-diol (5c) as a white solid. Yield: 0.130 g, 70.25%; LCMS: m/z 206.0 [M+H].

### Synthesis of 1-((3S,4R,5R,6R)-4,5-dihydroxy-6-(hydroxymethyl)tetrahydro-2H-pyran-3-yl)thiourea (5a)

A solution of (2R,3R,4R,5S)-2-(hydroxymethyl)-5-isothiocyanatotetrahydro-2H-pyran-3,4-diol (5c, 0.130 g, 0.633 mmol) and 7M ammonia in methanol (5 mL) was stirred at room temperature for 2h. Progress of the reaction was monitored by TLC and LCMS. After completion, the reaction mixture was concentrated under reduced pressure to obtain crude residue which was washed with diethyl ether (2-3 times) and dried under vacuum to afford 1-((3S,4R,5R,6R)-4,5-dihydroxy-6-(hydroxymethyl)tetrahydro-2H-pyran-3-yl)thiourea (5a) as a white solid which was directly used for the next step without further purification. Yield: 0.10 g, Crude; LCMS: m/z 223.0 [M+H].

### Synthesis of tert-butyl (2-(2-(2-(allyloxy)ethoxy)ethoxy)ethyl)carbamate (2)

To a solution of tert-butyl (2-(2-(2-hydroxyethoxy)ethoxy)ethyl)carbamate (1, 1.00 g, 4.01 mmol) in anhydrous tetrahydrofuran (10 mL), sodium hydride (0.176 g, 1.1 eq., 4.41 mmol) was added portion-wise at 0° C. After being stirred for 20 min, 3-bromopropene (0.381 mL, 1.1 eq., 4.41 mmol) was added and the reaction mixture was stirred for 2 h at 0° C. Then it was brought to room temperature and stirred for another 1 h. Progress of the reaction was monitored by TLC and LCMS. After completion, the reaction mixture was quenched with ice-cold water (10 mL) and organic part was extracted with dichloromethane (3×20 mL). Combined organic layer was dried over anhydrous sodium sulphate, filtered and the filtrate was concentrated under reduced pressure to obtain crude residue. The crude was purified by flash column chromatography using 0-40% ethyl acetate/heptane as eluting solvent to afford tert-butyl (2-(2-(2-(allyloxy)ethoxy)ethoxy)ethyl)carbamate (2) as a colourless viscous liquid. Yield: 0.750 g, 62.03%; LCMS-MS: m/z 290.1 [M+H].

### Synthesis of tert-butyl (2-(2-(2-(oxiran-2-ylmethoxy)ethoxy)ethoxy)ethyl)carbamate (3)

To a solution of tert-butyl (2-(2-(2-(allyloxy)ethoxy)ethoxy)ethyl)carbamate (2, 0.6 g, 2.07 mmol) in anhydrous dichloromethane (10 mL) under argon atmosphere, a solution of m-chloroperbenzoic acid (m-CPBA, 1.43 g, 4.0 eq., 8.29 mmol) in dichloromethane (4 mL) was added dropwise at 0° C. The solution was allowed to reach room temperature and stirred for 20h. Progress of the reaction was monitored by TLC and LCMS. After completion, saturated aqueous sodium bicarbonate solution was added to the reaction mixture and the aqueous layer was extracted with ethyl acetate for three times. Combined organic extracts were dried over anhydrous sodium sulfate, filtered and filtrate was concentrated in vacuo to give crude which was purified by flash column chromatography using 40% ethyl acetate in heptane as eluent to afford tert-butyl (2-(2-(2-(oxiran-2-ylmethoxy)ethoxy)ethoxy)ethyl)carbamate (3) as a light yellow oil. Yield: 0.250 g, 39.48%; LCMS: m/z 306.0 [M+H].

### Synthesis of tert-butyl (2-(2-(2-(3-bromo-2-hydroxypropoxy)ethoxy)ethoxy)ethyl)carbamate (4)

To a stirred solution of tert-butyl (2-(2-(2-(oxiran-2-ylmethoxy)ethoxy)ethoxy)ethyl)carbamate (3, 1.25 g, 4.09 mmol) in anhydrous tetrahydrofuran (50 mL), lithium bromide (1.42 g, 4 eq., 16.4 mmol) and acetic acid (0.709 mL, 3.0 eq., 12.3 mmol) was added at 0° C. and reaction mixture was stirred at room temperature for 1h. Progress of the reaction was monitored by TLC & LCMS. After completion, volatiles were removed under vacuum to obtain crude tert-butyl (2-(2-(2-(3-bromo-2-hydroxypropoxy)ethoxy)ethoxy)ethyl)carbamate (4, 1.35 g) which was directly used in the next step without further purification. Yield: 1.35 g, Crude; LCMS: m/z 387.75 [M+H]

### Synthesis of tert-butyl (2-(2-(2-(3-bromo-2-oxopropoxy)ethoxy)ethoxy)ethyl)carbamate (5)

To a solution of tert-butyl (2-(2-(2-(3-bromo-2-hydroxypropoxy)ethoxy)ethoxy)ethyl)carbamate (4, 1.35 g, 3.49 mmol) in dry dichloromethane (60 mL) at room temperature and under argon was added Dess-Martin periodinane (DMP, 4.45 g, 3.0 eq., 10.5 mmol). After 3 h, the reaction mixture was cooled down to 0° C., quenched with the addition of an aqueous sodium thiosulfate solution (1M) and saturated sodium bicarbonate solution. The aqueous layer was extracted with ethyl acetate. Combined organic extracts were washed with brine, dried over anhydrous sodium sulfate, filtered and the filtrate was concentrated in vacuo to give residue which was purified by flash column chromatography 40% using ethyl acetate in heptane as eluent to afford tert-butyl (2-(2-(2-(3-bromo-2-oxopropoxy)ethoxy)ethoxy)ethyl)carbamate (5) as a colorless oil. Yield: 0.750 g, 47.47%; LCMS: m/z 386.0 [M+H]

### Synthesis of tert-butyl (2-(2-(2-((2-(((3S,4R,5R,6R)-4,5-dihydroxy-6-(hydroxymethyl)tetrahydro-2H-pyran-3-yl)amino)thiazol-4-yl)methoxy)ethoxy)ethoxy)ethyl)carbamate (6)

A solution of tert-butyl (2-(2-(2-(3-bromo-2-oxopropoxy)ethoxy)ethoxy)ethyl)carbamate (5, 0.520 g, 1.0 eq, 1.15 mmol) and 1-((3S,4R,5R,6R)-4,5-dihydroxy-6-(hydroxymethyl)tetrahydro-2H-pyran-3-yl)thiourea (5a, 0.284 g, 1.15 mmol) in acetone (5 mL) was heated at 50° C. and the reaction mixture was stirred at same temperature for 3h. Progress of the reaction was monitored by TLC and LCMS. After completion of reaction, solvent was evaporated under reduced pressure and dried to obtain crude tert-butyl (2-(2-(2-((2-(((3S,4R,5R,6R)-4,5-dihydroxy-6-(hydroxymethyl)tetrahydro-2H-pyran-3-yl)amino)thiazol-4 -yl)methoxy)ethoxy)ethoxy)ethyl)carbamate (6, 1.2 g) which was directly used in the next step without further purification. LCMS: m/z 508.12 [M+H].

### Synthesis of (2R,3R,4R,5S)-5-((4-((2-(2-(2-aminoethoxy)ethoxy)ethoxy)methyl)thiazol-2-yl)amino)-2-(hydroxymethyl)tetrahydro-2H-pyran-3,4-diol (XB82)

To a stirred solution of tert-butyl (2-(2-(2-((2-(((3S,4R,5R,6R)-4,5-dihydroxy-6-(hydroxymethyl)tetrahydro-2H-pyran-3-yl)amino)thiazol-4-yl)methoxy)ethoxy)ethoxy)ethyl)carbamate (6, 1.2 g) in dichloromethane (10 mL), trifluoroacetic acid (10 mL) was added at 0° C. Then, reaction mixture was stirred at room temperature for 1h. Progress of the reaction was monitored by LCMS. After completion of reaction, solvent was evaporated under reduced pressure to obtain crude residue. Crude was purified by RP prep-HPLC (60% acetonitrile in water with 0.1% trifluoroacetic acid). Fractions containing desired product were combined and lyophilized to afford (2R,3R,4R,5S)-5-((4-((2-(2-(2-aminoethoxy)ethoxy)ethoxy)methyl)thiazol-2-yl)amino)-2-(hydroxymethyl)tetrahydro-2H-pyran-3,4-diol (XB82) as a white sticky solid. Yield: 0.376 g, 48.79%; LCMS m/z 408.25 [M+H]; ¹H-NMR (400 MHz, DMSO-d₆): δ 7.73 (brs, 3H), 6.58 (s, 1H), 4.28 (s, 2H), 3.94-3.91 (m, 1H), 3.83-3.82 (m, 1H), 3.74 (d, J=6.4 Hz, 1H), 3.59-3.52 (m, 10H), 3.51-3.42 (m, 3H), 3.28 (t, J=6.0 Hz, 1H), 3.00-2.95 (m, 3H).

### Synthesis of (2R,3R,4R,5S)-5-((4-(2-(2-(2-aminoethoxy)ethoxy)ethoxy)-6-(trifluoromethyl)pyrimidin-2-yl)amino)-2-(methoxymethyl)tetrahydro-2H-pyran-3,4-diol

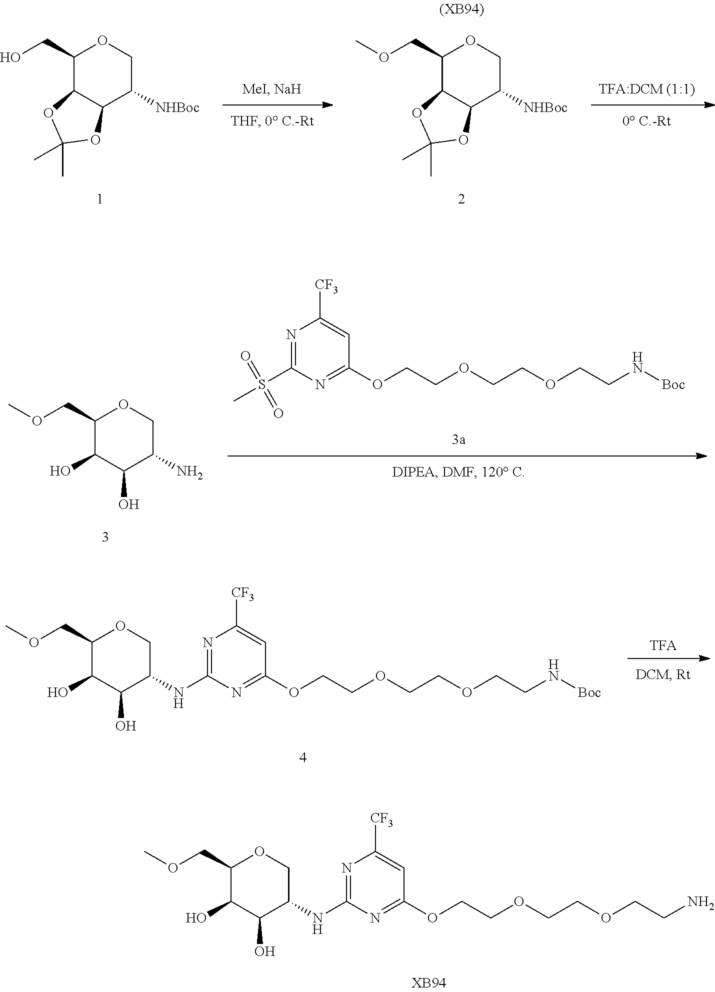

### Synthesis of tert-butyl ((3aR,4R,7S,7aR)-4-(methoxymethyl)-2,2-dimethyltetrahydro-4H-[1,3]dioxolo[4,5-c]pyran-7-yl)carbamate (2)

To a stirred solution of tert-butyl ((3aR,4R,7S,7aR)-4-(hydroxymethyl)-2,2-dimethyltetrahydro-4H-[1,3]dioxolo[4,5-c]pyran-7-yl)carbamate (1, 0.3 g, 1 eq., 0.989 mmol) in dry tetrahydrofuran (5 mL), sodium hydride (47.5 mg, 2 eq., 1.98 mmol) was added portion-wise at 0° C. After that, reaction mixture was stirred at room temperature for 30 minutes. Then, iodomethane (0.092 mL, 1.5 eq., 1.48 mmol) was added to the reaction mixture at 0° C. and stirred at room temperature for 30 minutes. After completion (monitored by ELSD-MS), reaction mixture was quenched with ice-cold water followed by concentration gave crude residue which was re-dissolved in ethyl acetate and poured in water, then extracted with ethyl acetate. The organic layer was dried over anhydrous sodium sulphate, filtered, and concentrated to give crude residue which was purified by silica gel column chromatography using 40% ethyl acetate in heptane as eluent to afford tert-butyl (3aR,4R,7S,7aR)-4-(methoxymethyl)-2,2-dimethyltetrahydro-4H-[1,3]dioxolo[4,5-c]pyran-7-yl)carbamate as off-white solid. Yield: 0.28 g, 89.0%. LCMS: m/z 318.10 [M+H].

### Synthesis of (2R,3R,4R,5S)-5-amino-2-(methoxymethyl)tetrahydro-2H-pyran-3,4-diol (3)

To a stirred solution of (3aR,4R,7S,7aR)-4-(methoxymethyl)-2,2-dimethyltetrahydro-4H-[1,3]dioxolo[4,5-c]pyran-7-yl)carbamate (0.28 g, 1 eq., 0.88 mmol) in dichloromethane (5 mL), trifluoroacetic acid (5 mL) was added to the reaction mixture at 0° C. Then, reaction mixture was stirred at room temperature for 2 h. After completion (monitored by ELSD-MS), reaction mixture was directly concentrated and washed with diethyl ether to get crude (2R,3R,4R,5S)-5-amino-2-(methoxymethyl)tetrahydro-2H-pyran-3,4-diol (3) as brown solid. Yield: 0.18 g, Crude. LCMS: m/z 178.2 [M+H].

### Synthesis of tert-butyl (2-(2-(2-((2-(((3S,4R,5R,6R)-4,5-dihydroxy-6-(methoxymethyl)tetrahydro-2H-pyran-3-yl)amino)-6-(trifluoromethyl)pyrimidin-4-yl)oxy)ethoxy)ethoxy)ethyl)carbamate (4)

To a stirred solution of (2R,3R,4R,5S)-5-amino-2-(methoxymethyl)tetrahydro-2H-pyran-3,4-diol (3, 0.185 g, 1 eq., 0.634 mmol) and tert-butyl (2-(2-(2-((2-(methylsulfonyl)-6-(trifluoromethyl)pyrimidin-4-yl)oxy)ethoxy)ethoxy)ethyl)carbamate (3a, 0.3 g, 1 eq., 0.634 mmol) in dry N,N-dimethylformamide (3 mL), was added N,N-Diisopropylethylamine (1.32 mL, 12 eq., 7.6 mmol) at 0° C. and allowed to stir at 120° C. for 6 h. After completion (monitored by LCMS), reaction mixture was concentrated to afford crude tert-butyl (2-(2-(2-((2-(((3S,4R,5R,6R)-4,5-dihydroxy-6-(methoxymethyl)tetrahydro-2H-pyran-3-yl)amino)-6-(trifluoromethyl)pyrimidin-4-yl)oxy)ethoxy)ethoxy)ethyl)carbamate (4) as brown liquid which was used for the next step without purification. Yield: 0.317 g, Crude. LCMS: m/z 571.15 [M+H].

### Synthesis of (2R,3R,4R,5S)-5-((4-(2-(2-(2-aminoethoxy)ethoxy)ethoxy)-6-(trifluoromethyl)pyrimidin-2-yl)amino)-2-(methoxymethyl)tetrahydro-2H-pyran-3,4-diol (XB94)

A solution of tert-butyl (2-(2-(2-((2-(((3S,4R,5R,6R)-4,5-dihydroxy-6-(methoxymethyl)tetrahydro-2H-pyran-3-yl)amino)-6-(trifluoromethyl)pyrimidin-4 -yl)oxy)ethoxy)ethoxy)ethyl)carbamate (4, 0.362 g, 1 eq., 0.634 mmol) in dichloromethane (3 mL) was cooled at 0° C. Then, trifluoroacetic acid (3 mL) was added to it and reaction mixture was stirred at room temperature for 3 h. After completion (monitored by LCMS), reaction mixture was concentrated under reduced pressure to afford crude which was purified by RP prep-HPLC (14% acetonitrile in water with 0.1% TFA). Fractions containing the desired product were collected and lyophilized to afford (2R,3R,4R,5S)-5-((4-(2-(2-(2-aminoethoxy)ethoxy)ethoxy)-6-(trifluoromethyl)pyrimidin-2-yl)amino)-2-(methoxymethyl)tetrahydro-2H-pyran-3,4-diol (XB94) as a white solid. Yield: 0.078 g, 30.19%. LCMS: m/z 471.30 [M+H]. ¹H NMR (400 MHz, DMSO-d₆, high temperature): δ 7.63 (brs, 3H), 7.20 (brs, 1H), 6.37 (brs, 1H), 4.47-4.48 (t, J=4.8 Hz, 2H), 4.14-4.06 (m, 1H), 3.94-3.89 (m, 1H), 3.77 (t, J=4.8 Hz, 2H), 3.73 (brs, 1H), 3.66-3.59 (m, 7H), 3.54-3.43 (m, 3H), 3.28 (s, 3H), 2.98 (t, J=5.2 Hz, 3H).

### Synthesis of (2R,3R,4R,5S)-5-((4-(2-(2-(2-aminoethoxy)ethoxy)ethoxy)-6-(trifluoromethyl)pyrimidin-2-yl)amino)-2-((4-methylpiperazin-1-yl)methyl)tetrahydro-2H-pyran-3,4-diol (XB92)

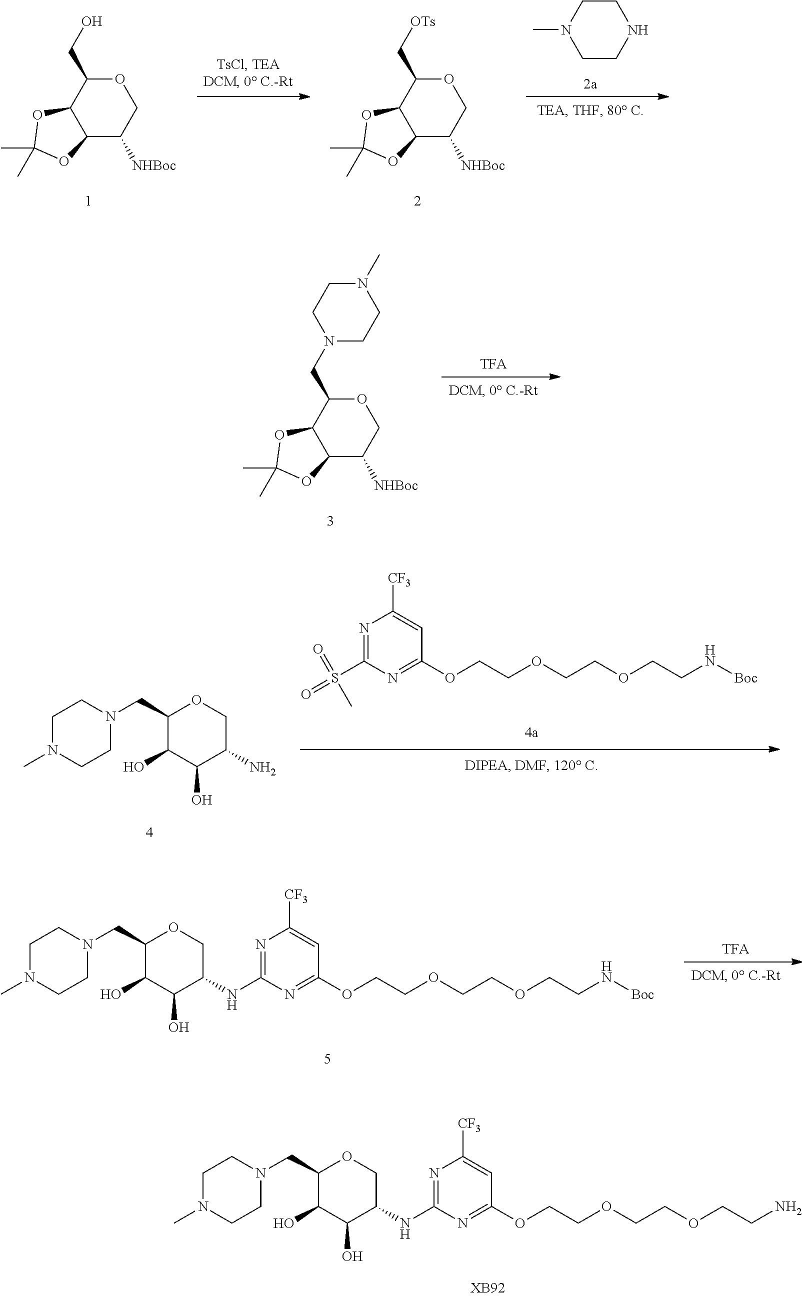

### Synthesis of ((3aR,4R,7S,7aR)-7-((tert-butoxycarbonyl)amino)-2,2-dimethyltetrahydro-4H-[1,3]dioxolo[4,5-c]pyran-4-yl)methyl 4-methylbenzenesulfonate (2)

To a solution of tert-butyl ((3aR,4R,7S,7aR)-4-(hydroxymethyl)-2,2-dimethyltetrahydro-4H-[1,3]dioxolo[4,5-c]pyran-7-yl)carbamate (1, 0.5 g, 1.65 mmol) in dichloromethane (8 mL), triethylamine (0.92 mL, 4 eq., 6.59 mmol) and 4-methylbenzenesulfonyl chloride (0.377 g, 1.2 eq., 1.98 mmol) were added at 0° C. The reaction mixture was stirred at room temperature for 2 h. After completion, the reaction mixture was diluted with water and extracted with dichloromethane. The combined organic layer was washed with water, dried over anhydrous sodium sulfate, filtered and concentrated to get crude which was purified by column chromatography using 10-25% ethyl acetate/heptane as eluent to afford ((3aR,4R,7S,7aR)-7-((tert-butoxycarbonyl)amino)-2,2-dimethyltetrahydro-4H-[1,3]dioxolo[4,5-c]pyran-4-yl)methyl 4-methylbenzenesulfonate (2) as off-white solid. Yield: 0.50 g, 66.3% LCMS: m/z 458.1 [M+H]

### Synthesis of tert-butyl ((3aS,4R,7S,7aR)-2,2-dimethyl-4-((4-methylpiperazin-1-yl)methyl)tetrahydro-4H-[1,3]dioxolo[4,5-c]pyran-7-yl)carbamate (3)

To a stirred solution of ((3aR,4R,7S,7aR)-7-((tert-butoxycarbonyl)amino)-2,2-dimethyltetrahydro-4H-[1,3]dioxolo[4,5-c]pyran-4-yl)methyl 4-methylbenzenesulfonate (2, 0.413 g, 1.0 eq., 0.903 mmol) in tetrahydrofuran (6 mL) was added triethylamine (0.381 mL, 3 eq., 2.71 mmol) and 1-methylpiperazine (2a, 0.181 g, 2 eq., 1.81 mmol) at 0° C. The resultant reaction mixture was stirred at 80° C. for 12 h. After completion, the reaction mixture was poured into water, and extracted with dichloromethane. The combined organic layer was washed with water, dried over anhydrous sodium sulfate, filtered and concentrated to obtain crude which was purified by column chromatography using 0-5% methanol in dichloromethane to afford tert-butyl ((3aS,4R,7S,7aR)-2,2-dimethyl-4-((4-methylpiperazin-1-yl)methyl)tetrahydro-4H-[1,3]dioxolo[4,5-c]pyran-7-yl)carbamate (3) as viscous brown liquid. Yield: 0.20 g, 57.4% LCMS: m/z 386.2 [M+H].

### Synthesis (2R,3R,4R,5S)-5-amino-2-((4-methylpiperazin-1-yl)methyl)tetrahydro-2H-pyran-3,4-diol (4)

To a solution of tert-butyl ((3aS,4R,7S,7aR)-2,2-dimethyl-4-((4-methylpiperazin-1-yl)methyl)tetrahydro-4H-[1,3]dioxolo[4,5-c]pyran-7-yl)carbamate (3, 0.3 g, 1.0 eq., 0.778 mmol) in dichloromethane (2.0 mL) was added trifluoroacetic acid (2 mL) at 0° C. and reaction mixture was stirred at room temperature for 3 h. After completion, the reaction mixture was concentrated under reduced pressure and co-evaporated with dichloromethane to afford (2R,3R,4R,5S)-5-amino-2-((4-methylpiperazin-1-yl)methyl)tetrahydro-2H-pyran-3,4-diol (4) as brown semi-solid. Yield: 0.20 g, LCMS: m/z 246.1 [M+H].

### Synthesis of tert-butyl (2-(2-(2-((2-(((3S,4R,5R,6R)-4,5-dihydroxy-6-((4-methylpiperazin-1-yl)methyl)tetrahydro-2H-pyran-3-yl)amino)-6-(trifluoromethyl)pyrimidin-4-yl)oxy)ethoxy)ethoxy)ethyl)carbamate (5)

To a stirred solution of (2R,3R,4R,5S)-5-amino-2-((4-methylpiperazin-1-yl)methyl)tetrahydro-2H-pyran-3,4-diol (4, 0.364 g, 1.2 eq., 0.845 mmol) and tert-butyl (2-(2-(2-((2-(methylsulfonyl)-6-(trifluoromethyl)pyrimidin-4-yl)oxy)ethoxy)ethoxy)ethyl)carbamate (4a, 0.4 g, 1 eq., 0.845 mmol) in dry N,N-dimethylformamide (4 mL) was added N,N-diisopropylethylamine (1.77 mL, 12 eq.) at 0° C. The reaction was stirred at 80° C. for 5 h. After completion, the reaction mixture was concentrated under reduced pressure to afford crude tert-butyl (2-(2-(2-((2-(((3S,4R,5R,6R)-4,5-dihydroxy-6-((4-methylpiperazin-1-yl)methyl)tetrahydro-2H-pyran-3-yl)amino)-6 -(trifluoromethyl)pyrimidin-4-yl)oxy)ethoxy)ethoxy)ethyl)carbamate (5) as brown semi-solid. Yield: 0.50 g, LCMS: m/z 639.3 [M+H]. The crude material was used for the next step without purification.

### Synthesis of (2R,3R,4R,5S)-5-((4-(2-(2-(2-aminoethoxy)ethoxy)ethoxy)-6-(trifluoromethyl)pyrimidin-2-yl)amino)-2-((4-methylpiperazin-1-yl)methyl)tetrahydro-2H-pyran-3,4-diol (XB92)

To a stirred solution of tert-butyl (2-(2-(2-((2-(((3S,4R,5R,6R)-4,5-dihydroxy-6-((4-methylpiperazin-1-yl)methyl)tetrahydro-2H-pyran-3-yl)amino)-6-(trifluoromethyl)pyrimidin-4-yl)oxy)ethoxy)ethoxy)ethyl)carbamate (5, 0.27 g, 1.0 eq., 0.42 mmol) in dichloromethane (0.2 mL) was added trifluoroacetic acid (0.2 mL) at 0° C. and reaction was stirred at room temperature for 4 h. After completion of reaction as indicated by LCMS, reaction mixture was concentrated under reduce pressure to get crude. Crude was purified by RP prep HPLC (80% acetonitrile in water with 0.10% TFA) to afford (2R,3R,4R,5S)-5-((4-(2-(2-(2-aminoethoxy)ethoxy)ethoxy)-6-(trifluoromethyl)pyrimidin-2-yl)amino)-2-((4-methylpiperazin-1-yl)methyl)tetrahydro-2H-pyran-3,4-diol (XB92) as white sticky solid. Yield: 0.094 g; 22.3%, LCMS: m/z 539.2 [M+H]; ¹H-NMR (400 MHz, DMSO-d₆-D2O exchange, high temperature): δ 6.37 (s, 1H), 4.43 (brs, 2H), 4.12-4.07 (m, 1H), 3.90-3.86 (m, 1H), 3.74 (t, J=4.4 Hz, 2H), 3.71 (brs, 1H), 3.61-3.57 (m, 9H), 3.08-3.03 (m, 6H), 2.97 (t, J=4.8 Hz, 2H), 2.91-2.80 (m, 4H), 2.78-2.75 (m, 2H), 2.68-2.67 (m, 3H).

### Synthesis of N-((2S,3R,4R,5R,6R)-2-(1-(2-(2-(2-aminoethoxy)ethoxy)ethyl)-1H-1,2,3-triazol-4-yl)-4,5-dihydroxy-6-(hydroxymethyl)tetrahydro-2H-pyran-3-yl)acetamide (XB97)

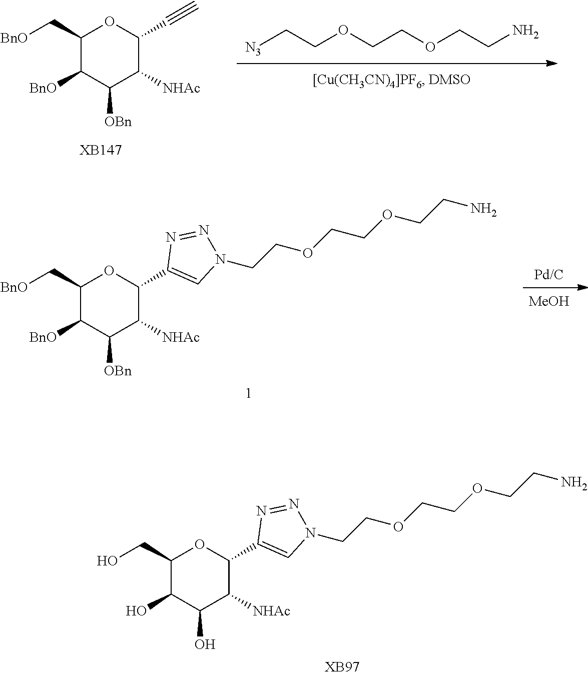 [SMILES: "MeOH" ; C#C[C@H]1O[C@H](COCc2ccccc2)[C@H](OCc2ccccc2)[C@H](OCc2ccccc2)[C@H]1NC(C)=O ; [N-]=[N+]=NCCOCCOCCN ; CC(=O)N[C@@H]1[C@@H](OCc2ccccc2)[C@@H](OCc2ccccc2)[C@@H](COCc2ccccc2)O[C@@H]1c1cn(CCOCCOCCN)nn1 ; "Pd/C" ; CC(=O)N[C@@H]1[C@@H](O)[C@@H](O)[C@@H](CO)O[C@@H]1c1cn(CCOCCOCCN)nn1]

### Synthesis of N-((2S,3S,4R,5R,6R)-2-(1-(2-(2-(2-aminoethoxy)ethoxy)ethyl)-1H-1,2,3-triazol-4-yl)-4,5-bis(benzyloxy)-6-((benzyloxy)methyl)tetrahydro-2H-pyran-3-yl)acetamide (1)

To a solution of N-[(2R,3S,4R,5R,6R)-4,5-dibenzyloxy-6-(benzyloxymethyl)-2-ethynyl-tetrahydropyran-3-yl]acetamide (XB147, 1.00 eq, 46.0 mg, 0.092 mmol) in DMSO (0.25 mL) was added azido-PEG2-amine (1.20 eq, 19.2 mg, 0.11 mmol) and tetrakis(acetonitrile)copper(I) hexafluorophosphate (1.50 eq, 51.5 mg, 0.14 mmol). The mixture was stirred at rt for 1 h and purified by prep. HPLC (5-70% MeCN/water with 0.1% TFA) to give 1 as a white solid (54 mg, yield: 87%). LCMS m/z 674.4 [M+H].

### Synthesis of N-((2S,3R,4R,5R,6R)-2-(1-(2-(2-(2-aminoethoxy)ethoxy)ethyl)-1H-1,2,3-triazol-4-yl)-4,5-dihydroxy-6-(hydroxymethyl)tetrahydro-2H-pyran-3-yl)acetamide (XB97)

To a mixture of N-((2S,3S,4R,5R,6R)-2-(1-(2-(2-(2-aminoethoxy)ethoxy)ethyl)-1H-1,2,3-triazol-4-yl)-4,5-bis(benzyloxy)-6-((benzyloxy)methyl)tetrahydro-2H-pyran-3-yl)acetamide (1.00 eq, 54.0 mg, 0.080 mmol) in MeOH (4 mL) was added 10% Pd/C (170 mg). The mixture was stirred at rt under hydrogen for 7h, filtered, concentrated, and purified by prep. HPLC (3-25% MeCN/20 mM NH₄OH solution) to give XB97 as a white solid (19.6 mg, yield: 61%). LCMS: 404.3 [M+H].

### Synthesis of (2R,3R,4R,5R,6R)-5-((4-(2-(2-(2-aminoethoxy)ethoxy)ethoxy)pyrimidin-2-yl)amino)-2-(methoxymethyl)-6-propyltetrahydro-2H-pyran-3,4-diol (XB83)

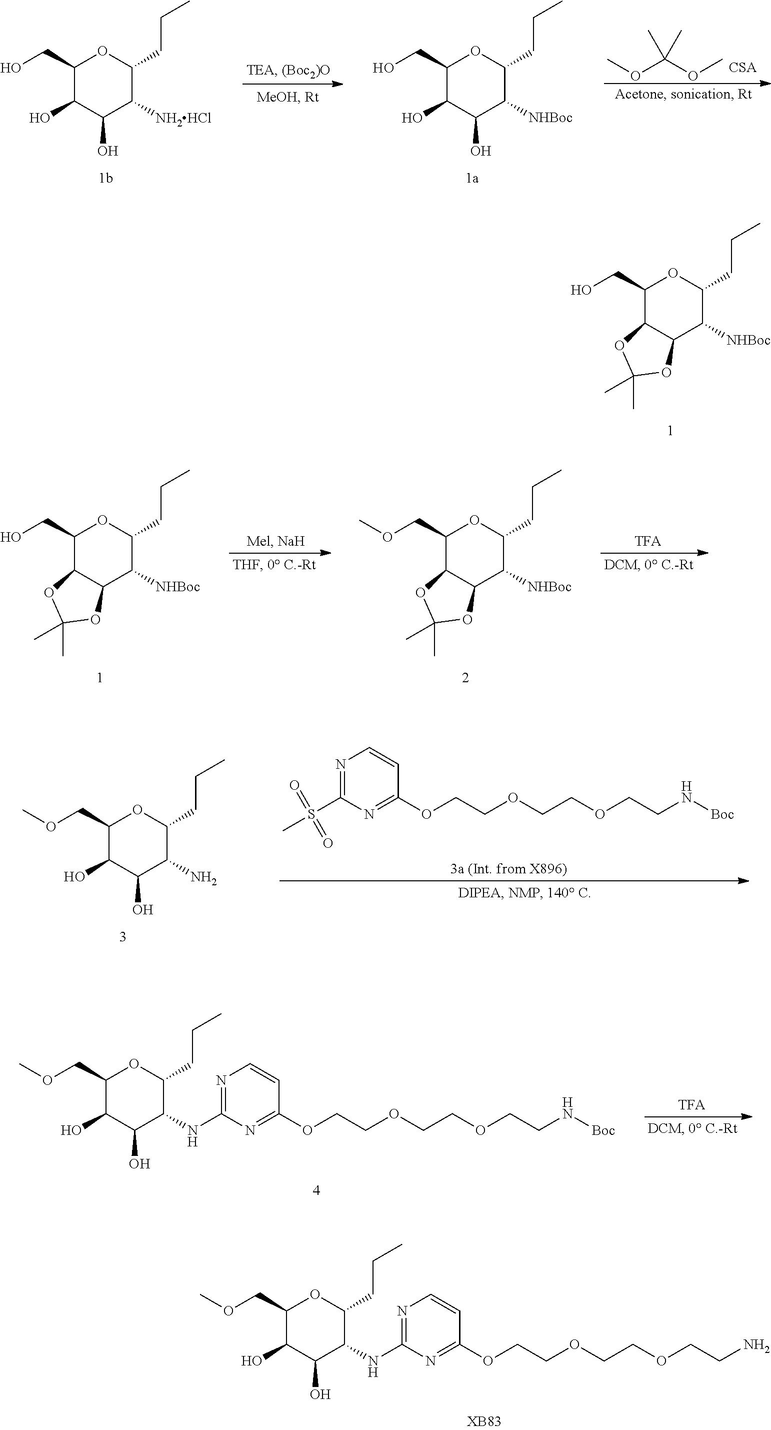

### Tert-butyl ((2R,3R,4R,5R,6R)-4,5-dihydroxy-6-(hydroxymethyl)-2-propyltetrahydro-2H-pyran-3-yl)carbamate (1a)

To a stirred solution of (2R,3R,4R,5R,6R)-5-amino-2-(hydroxymethyl)-6-propyltetrahydro-2H-pyran-3,4-diol hydrochloride (1b, 5.3 g, 21.9 mmol) in methanol (45 mL), were added triethylamine (3 eq., 9 mL, 65.8 mmol) and di-tert-butyl dicarbonate (1.2 eq., 6.04 mL, 26.3 mmol) gradually at 0° C. under nitrogen atmosphere. Then reaction mixture was stirred at room temperature for 12 h. After completion the reaction mixture was concentrated under reduced pressure to get a crude which was diluted with water and extracted with ethyl acetate (5×120 mL). Organic part was dried over anhydrous sodium sulphate, filtered and concentrated under reduced pressure to afford tert-butyl ((2R,3R,4R,5R,6R)-4,5-dihydroxy-6-(hydroxymethyl)-2-propyltetrahydro-2H-pyran-3-yl)carbamate (1a) as off white solid. Yield: 5.0 g, crude; LCMS m/z 306.1[M+H].

### tert-butyl ((3aR,4R,6R,7S,7aR)-4-(hydroxymethyl)-2,2-dimethyl-6-propyltetrahydro-4H-[1,3]dioxolo[4,5-c]pyran-7-yl)carbamate (1)

To a stirred solution of tert-butyl ((2R,3R,4R,5R,6R)-4,5-dihydroxy-6-(hydroxymethyl)-2-propyltetrahydro-2H-pyran-3-yl)carbamate (1a, 1.0 eq., 3.0 g, 9.82 mmol) in 2,2-dimethoxypropane (15.0 mL, 22 eq., 218 mmol) and acetone (15.0 mL) was added camphor sulfonic acid (0.2 eq., 0.454 g, 1.96 mmol) at 0° C. and reaction mixture was sonicated for 30 min. Then 0.5 mL of methanol was added to the reaction mixture and reaction was stirred at room temperature for 10 min. After completion of reaction, reaction mixture was neutralized using triethyl amine and concentrated under reduced pressure to afford crude which was purified by silica gel flash column chromatography using 20-50% ethyl acetate/hexane as eluent to afford tert-butyl ((3aR,4R,6R,7S,7aR)-4-(hydroxymethyl)-2,2-dimethyl-6-propyltetrahydro-4H-[1,3]dioxolo[4,5-c]pyran-7-yl)carbamate (1) as off white solid. Yield: 1.6 g, 47%; ELSD-MS m/z 346.2 [M+H]⁺.

### Synthesis of tert-butyl ((3aR,4R,6R,7S,7aR)-4-(methoxymethyl)-2,2-dimethyl-6-propyltetrahydro-4H-[1,3]dioxolo[4,5-c]pyran-7-yl)carbamate (2)

To a stirred solution of tert-butyl ((3aR,4R,6R,7S,7aR)-4-(hydroxymethyl)-2,2-dimethyl-6-propyltetrahydro-4H-[1,3]dioxolo[4,5-c]pyran-7-yl)carbamate (1, 1.0 g, 1 eq., 2.89 mmol) in dry tetrahydrofuran (15 mL), sodium hydride (127 mg, 1.1 eq., 3.18 mmol) was added portion-wise at 0° C. under nitrogen atmosphere. The reaction was allowed to stir at room temperature for 30 min. After that, methyl iodide (0.360 mL, 2 eq., 5.79 mmol) was added slowly at 0° C., and stirred at room temperature for another 30 min. After completion (monitored by TLC), ice-cold water was added to the reaction mixture and extracted with dichloromethane (3×10 mL). Combined organic portions were dried over anhydrous sodium sulphate and concentrated under reduced pressure to give crude residue which was purified by silica gel flash column chromatography using 40% ethyl acetate in heptane as the eluent to afford tert-butyl ((3aR,4R,6R,7S,7aR)-4-(methoxymethyl)-2,2-dimethyl-6-propyltetrahydro-4H-[1,3]dioxolo[4,5-c]pyran-7-yl)carbamate (2) as yellow sticky liquid. Yield: 0.75 g, 72.0%. LCMS m/z 360.20 [M+H].

### Synthesis of (2R,3R,4R,5R,6R)-5-amino-2-(methoxymethyl)-6-propyltetrahydro-2H-pyran-3,4-diol (3)

To a stirred solution of ((3aR,4R,6R,7S,7aR)-4-(methoxymethyl)-2,2-dimethyl-6-propyltetrahydro-4H-[1,3]dioxolo[4,5-c]pyran-7-yl)carbamate (2, 1.1 g, 1.0 eq., 3.06 mmol) in dichloromethane (15 mL), trifluoroacetic acid (15 mL) was added dropwise at 0° C. The reaction mixture was stirred at room temperature for 4 h. After completion (monitored by TLC), reaction mixture was concentrated under reduce pressure and co-evaporated with dichloromethane three times to obtain crude residue, which was further lyophilized to afford (2R,3R,4R,5R,6R)-5-amino-2-(methoxymethyl)-6-propyltetrahydro-2H-pyran-3,4-diol (3) as light yellow syrup. The crude residue was directly used for next step. Yield: 0.6 g (Crude). LCMS m/z 220.1 [M+H]⁺.

### Synthesis of tert-butyl (2-(2-(2-((2-(((2R,3R,4R,5R,6R)-4,5-dihydroxy-6-(methoxymethyl)-2-propyltetrahydro-2H-pyran-3-yl)amino)pyrimidin-4-yl)oxy)ethoxy)ethoxy)ethyl)carbamate (4)

To a stirred solution of (2R,3R,4R,5R,6R)-5-amino-2-(methoxymethyl)-6-propyltetrahydro-2H-pyran-3,4-diol (3, 0.7 g, 1.2 eq., 2.1 mmol) in dry 1-methyl-2-pyrrolidinone (2 mL), N,N-Diisopropylethylamine (3.66 mL, 12 eq., 21 mmol) was added at room temperature and then heated at 140° C. for 16 h. After completion (monitored by ELSD-MS), reaction mixture was concentrated under reduced pressure. The crude residue was purified by silica gel flash column chromatography using 2-3% methanol in dichloromethane as the eluent to afford tert-butyl (2-(2-(2-((2-(((2R,3R,4R,5R,6R)-4,5-dihydroxy-6-(methoxymethyl)-2-propyltetrahydro-2H-pyran-3-yl)amino)pyrimidin-4-yl)oxy)ethoxy)ethoxy)ethyl)carbamate (4) as brown gummy liquid. Yield: 0.64 g, 67.1%. LCMS m/z 545.90 [M+H]⁺.

### Synthesis of (2R,3R,4R,5R,6R)-5-((4-(2-(2-(2-aminoethoxy)ethoxy)ethoxy)pyrimidin-2-yl)amino)-2-(methoxymethyl)-6-propyltetrahydro-2H-pyran-3,4-diol (XB83)

A solution of tert-butyl (2-(2-(2-((2-(((2R,3R,4R,5R,6R)-4,5-dihydroxy-6-(methoxymethyl)-2-propyltetrahydro-2H-pyran-3-yl)amino)pyrimidin-4-yl)oxy)ethoxy)ethoxy)ethyl)carbamate (4, 0.140 g, 1 eq., 0.257 mmol) in dichloromethane (1 mL) was cooled at 0° C. and trifluoroacetic acid (1 mL) was added and the reaction mixture was stirred at room temperature for 3 h. After completion, the reaction mixture was concentrated under reduce pressure to get crude residue which was purified by RP prep HPLC (25% acetonitrile in water with 0.1% TFA) to afford ((2R,3R,4R,5R,6R)-5-((4-(2-(2-(2-aminoethoxy)ethoxy)ethoxy)pyrimidin-2-yl)amino)-2-(methoxymethyl)-6-propyltetrahydro-2H-pyran-3,4-diol (XB83) as colorless sticky solid. Yield: 0.058 g, 50.8%. LCMS m/z 445.3 [M+H]⁺. ¹H NMR (400 MHz, DMSO-D₆ with D₂O): δ 7.99 (d, J=6.4 Hz, 1H), 6.18 (d, J=6.0 Hz, 1H), 4.44-4.43 (m, 2H), 4.18-4.14 (m, 1H), 4.10-4.06 (m, 1H), 3.75-3.70 (m, 5H), 3.60-3.58 (m, 6H), 3.50 (d, J=4.4 Hz, 1H), 3.25 (s, 3H), 2.97 (t, J=5.2 Hz, 2H), 1.61-1.55 (m, 1H), 1.33-1.18 (m, 4H), 0.81 (t, J=6.8 Hz, 3H).

### Synthesis of (1S,2R,3R,4R,5S)-4-(4-((2-(2-(2-aminoethoxy)ethoxy)ethoxy)methyl)-1H-1,2,3-triazol-1-yl)-1-(hydroxymethyl)-6,8-dioxabicyclo[3.2.1]octane-2,3-diol TFA salt (XB57)

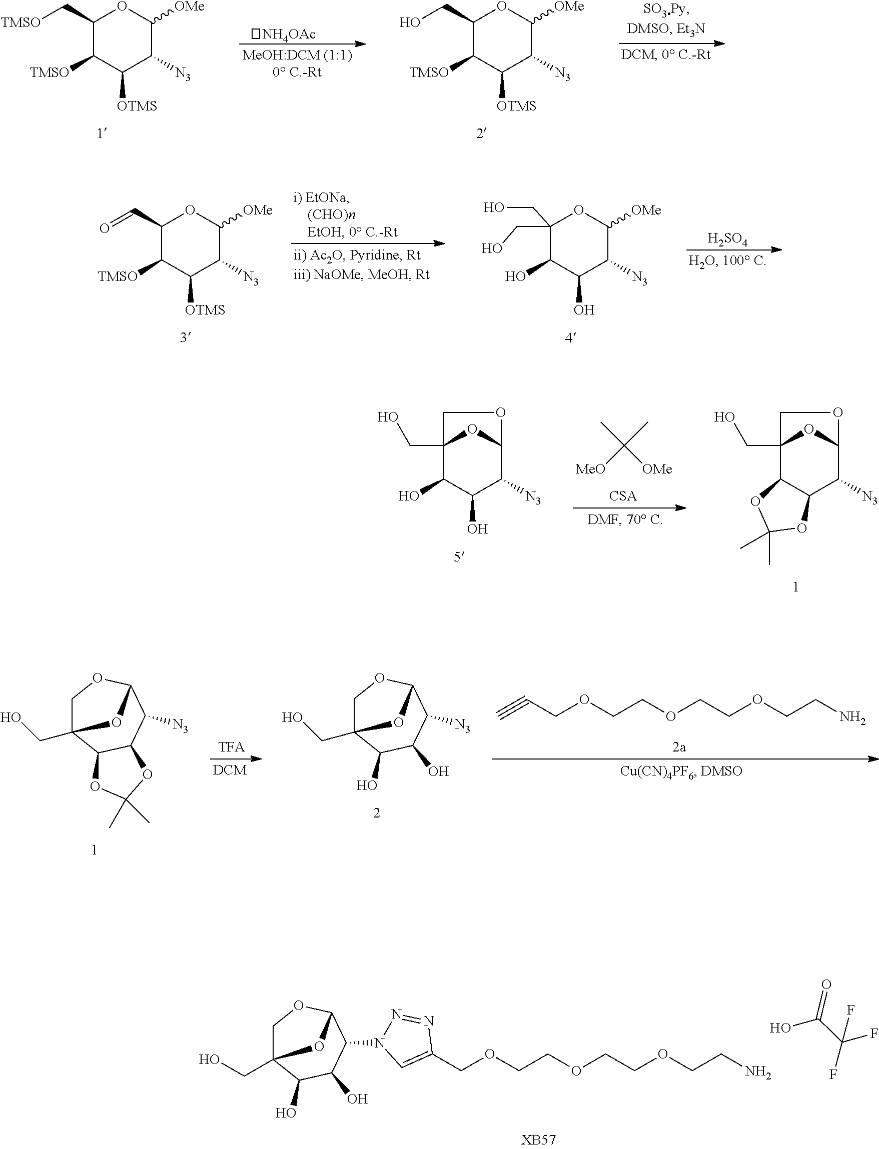

### Synthesis of ((2R,3S,4R,5R)-5-azido-6-methoxy-3,4-bis((trimethylsilyl)oxy)tetrahydro-2H-pyran-2-yl)methanol (2′)

To a stirred solution of (((2R,3S,4R,5R)-5-azido-6-methoxy-2-(((trimethylsilyl)oxy)methyl)tetrahydro-2H-pyran-3,4-diyl)bis(oxy))bis(trimethylsilane) (1′, 1.0 eq., 12.0 g, 27.5 mmol) in mixture of dichloromethane:methanol (1:1 ratio, 120 mL), ammonium acetate (1.5 eq., 12 g, 41.3 mmol) was added at room temperature under nitrogen. The resulting mixture was stirred at room temperature under nitrogen for 12 h. After completion (monitored by TLC & ELSD-MS) solvents were evaporated under reduced pressure and purified by column chromatography using silica gel (100-200 mesh) and 0-40% ethyl acetate in hexane to afford ((2R,3S,4R,5R)-5-azido-6-methoxy-3,4-bis((trimethylsilyl)oxy)tetrahydro-2H-pyran-2-yl)methanol (2) as a white solid. Yield: 8.0 g, 80.0%; LCMS m/z 364.15 [M+H].

### Synthesis of (2S,3S,4R,5R)-5-azido-6-methoxy-3,4-bis((trimethylsilyl)oxy)tetrahydro-2H-pyran-2-carbaldehyde (3′)

A solution of ((2R,3S,4R,5R)-5-azido-6-methoxy-3,4-bis((trimethylsilyl)oxy)tetrahydro-2H-pyran-2-yl)methanol (2′, 1.0 eq., 5.7 g, 15.7 mmol) in dichloromethane (60 mL) was treated with dimethylsulfoxide (7.5 eq., 8.35 mL, 118 mmol), and triethylamine (3.0 eq., 6.61 mL, 47 mmol) and the mixture was cooled to 0° C. Sulfur trioxide pyridine complex (3 eq., 7.49 g 47 mmol) was then added and the mixture was stirred at 0° C. for 1 h and then warmed to room temperature over 30 minutes. After completion, the reaction was quenched with brine and diluted with dichloromethane The aqueous. phase was extracted three times with dichloromethane and the combined organic layers were washed with brine, dried, filtered and evaporated to afford the corresponding crude (2S,3S,4R,5R)-5-azido-6-methoxy-3,4-bis((trimethylsilyl)oxy)tetrahydro-2H-pyran-2-carbaldehyde (3′) as pale yellow liquid. Yield: 5.4 g (crude); LCMS m/z 362.15 [M+H].

### Synthesis of (3R,4R,5R)-5-azido-2,2-bis(hydroxymethyl)-6-methoxytetrahydro-2H-pyran-3,4-diol (4′)

To a stirred solution of crude (2S,3S,4R,5R)-5-azido-6-methoxy-3,4-bis((trimethylsilyl)oxy)tetrahydro-2H-pyran-2-carbaldehyde (3′, 1.0 eq., 8.0 g 22.2 mmol), in anhydrous ethanol (70 mL) was treated with paraformaldehyde (30 eq., 19.9 g, 0.664 mol) followed by sodium ethoxide (21% in ethanol) (2.2 eq., 15.8 mL, 48.7 mmol). The reaction mixture was then stirred at room temperature 12 h. After completion (monitored by TLC) then reaction mixture was concentrated, the resulting crude was dried under vacuum and crude was washed with dichloromethane several times to afford crude residue which was dried and treated with acetic anhydride and pyridine (50 mL, 2:3, v/v). After being stirred at room temperature for overnight, reaction mixture was poured into 1N HCl, extracted with ethyl acetate. The organic layer was washed with sodium bicarbonate and dried over anhydrous sodium sulphate, filtered and concentrated to give crude which was purified by column chromatography using silica gel (100-200 mesh) and 0-50% ethyl acetate in hexane. The desired fractions were concentrated to afford corresponding tetraacetate which was again dissolved in methanol (50 mL), and treated with sodium methoxide (25% solution, 2.4 mL, 10.5 mmol) and then stirred for 3 h at room temperature.

After completion (monitored by TLC), the reaction mixture was quenched with Dowex H+ and filtered off The filtrate was concentrated under reduced pressure to get (3R,4R,5R)-5-azido-2,2-bis(hydroxymethyl)-6-methoxytetrahydro-2H-pyran-3,4-diol (4′) as brownish viscous liquid. Yield: 2.5 g, 41.0%; LCMS m/z 248.00 [M−1]⁻.

### Synthesis of (1S,2R,3R,4R,5S)-4-azido-1-(hydroxymethyl)-6,8-dioxabicyclo[3.2.1]octane-2,3-diol (5′)

To a stirred solution of ((3R,4R,5R)-5-azido-2,2-bis(hydroxymethyl)-6-methoxytetrahydro-2H-pyran-3,4-diol (4′, 1.0 eq., 1.0 g, 4.01 mmol) in water (10 mL) was added concentrated sulfuric acid (10.0 eq., 2.15 mL, 40.1 mmol) drop wise and the reaction mixture was stirred at 100° C. for 24 h. After completion (monitored by TLC), reaction mixture was cool to room temperature, and then neutralize by concentrated ammonium hydroxide. Thereafter, the resulting solution was evaporated to give crude residue which was again treated with methanol, filtered, and the filtrate was evaporated to get crude product which was purified by column chromatography using silica gel (100-200 mesh) and 0-10% methanol in dichloromethane to afford (1S,2R,3R,4R,5S)-4-azido-1-(hydroxymethyl)-6,8-dioxabicyclo[3.2.1]octane-2,3-diol (5′) Yield: 0.6 g, 68.88%; LCMS m/z 216.0 [M−1]⁻

### Synthesis of ((3aR,4S,7S,8R,8aR)-8-azido-2,2-dimethyltetrahydro-4,7-epoxy[1,3]dioxolo[4,5-d]oxepin-4(5H)-yl)methanol (1)

To a stirred solution of (1S,2R,3R,4R,5S)-4-azido-1-(hydroxymethyl)-6,8-dioxabicyclo[3.2.1]octane-2,3-diol (5′, 1.0 eq., 0.5 g, 1.94 mmol) in dimethylformamide (5.0 mL) and 2,2-dimethoxypropane (1.00 mL), was added camphorsulfonic acid (0.5 eq., 0.226 g, 0.97 mmol), and the resulting mixture was stirred at 70° C. temperature under nitrogen for 24 h After completion (monitored by TLC), reaction mixture was allowed to come to room temperature, and then triethylamine (1.0 mL) was added to reaction mixture. Then reaction mixture was partitioned between ethyl acetate and water. The water layer was extracted again with ethyl acetate. The combined organic layers were dried over sodium sulfate, filtered and concentrated under vacuum to give crude which was purified by silica gel chromatography (0 to 50% ethyl acetate in hexane) to afford ((3aR,4S,7S,8R,8aR)-8-azido-2,2-dimethyltetrahydro-4,7-epoxy[1,3]dioxolo[4,5-d]oxepin-4(5H)-yl)methanol (1) as sticky brown liquid. Yield: 0.25 g, 44.85%; LCMS m/z 258.10 [M+H]; ¹H NMR (400 MHz, DMSO-d₆ with D₂O exchange) δ 5.38 (d, J=1.6 Hz, 1H), 4.28-4.23 (m, 2H), 3.84-3.79 (m, 1H), 3.75-3.57 (m, 3H), 3.33 (dd, J=6.4, 2.0 Hz, 1H), 1.40 (s, 3H), 1.27 (s, 3H).

### Synthesis of (1S,2R,3R,4R,5S)-4-azido-1-(hydroxymethyl)-6,8-dioxabicyclo[3.2.1]octane-2,3-diol (2)

To a solution of ((3aR,4S,7S,8R,8aR)-8-azido-2,2-dimethyltetrahydro-4,7-epoxy[1,3]dioxolo[4,5-d]oxepin-4(5H)-yl)methanol (1) (58.2 mg, 0.226 mmol) in 1.2 mL DCM was added 0.3 mL of trifluoroacetic acid. The reaction was monitored by TLC until completion, then solvent evaporated under a stream of nitrogen. The residue was dissolved in a solution of acetonitrile and water, then frozen and lyophilized to dryness to afford (1S,2R,3R,4R,5S)-4-azido-1-(hydroxymethyl)-6,8-dioxabicyclo[3.2.1]octane-2,3-diol as a tan color solid. Yield: 31.0 mg (106%); LCMS m/z 219.8 [M+H].

### Synthesis of (1S,2R,3R,4R,5S)-4-(4-((2-(2-(2-aminoethoxy)ethoxy)ethoxy)methyl)-1H-1,2,3-triazol-1-yl)-1-(hydroxymethyl)-6,8-dioxabicyclo[3.2.1]octane-2,3-diol 2,2,2-trifluoroacetic acid (XB57)

To a solution of 2-(2-(2-(prop-2-yn-1-yloxy)ethoxy)ethoxy)ethan-1-amine (2a) (39.7 mg, 0.183 mmol, 1.0 eq.) and (1S,2R,3R,4R,5S)-4-azido-1-(hydroxymethyl)-6,8-dioxabicyclo[3.2.1]octane-2,3-diol (2) (34.4 mg, 0.184 mmol, 1.0 eq.) in 0.4 mL of dimethyl sulfoxide was added cuprous; acetonitrile; hexafluorophosphate (67.4 mg, 0.181 mmol, 0.99 eq) as a solid in one portion. The mixture was stirred under nitrogen atmosphere at ambient temperature for approximately 15 minutes until completion. The reaction mixture was diluted with water, which formed a precipitate, then 2 drops of trifluoroacetic acid was added to clear the solution. The product was isolated from the diluted mixture by preparatory HPLC, eluting with 1-20% acetonitrile in water with 0.1% trifluoroacetic acid. Fractions containing the desired product were combined and lyophilized to dryness to afford (1S,2R,3R,4R,5S)-4-(4-((2-(2-(2-aminoethoxy)ethoxy)ethoxy)methyl)-1H-1,2,3-triazol-1-yl)-1-(hydroxymethyl)-6,8-dioxabicyclo[3.2.1]octane-2,3-diol 2,2,2-trifluoroacetic acid (XB57) as a clear oil. Yield: 47.9 mg (50%); LCMS m/z 405.3 [M+H].

### Synthesis of (2R,3R,4R,5S)-5-((6-(2-(2-(2-(2-aminoethoxy)ethoxy)ethoxy)-1,1-difluoroethyl)pyrazin-2-yl)amino)-2-(hydroxymethyl)tetrahydro-2H-pyran-3,4-diol (XB80)

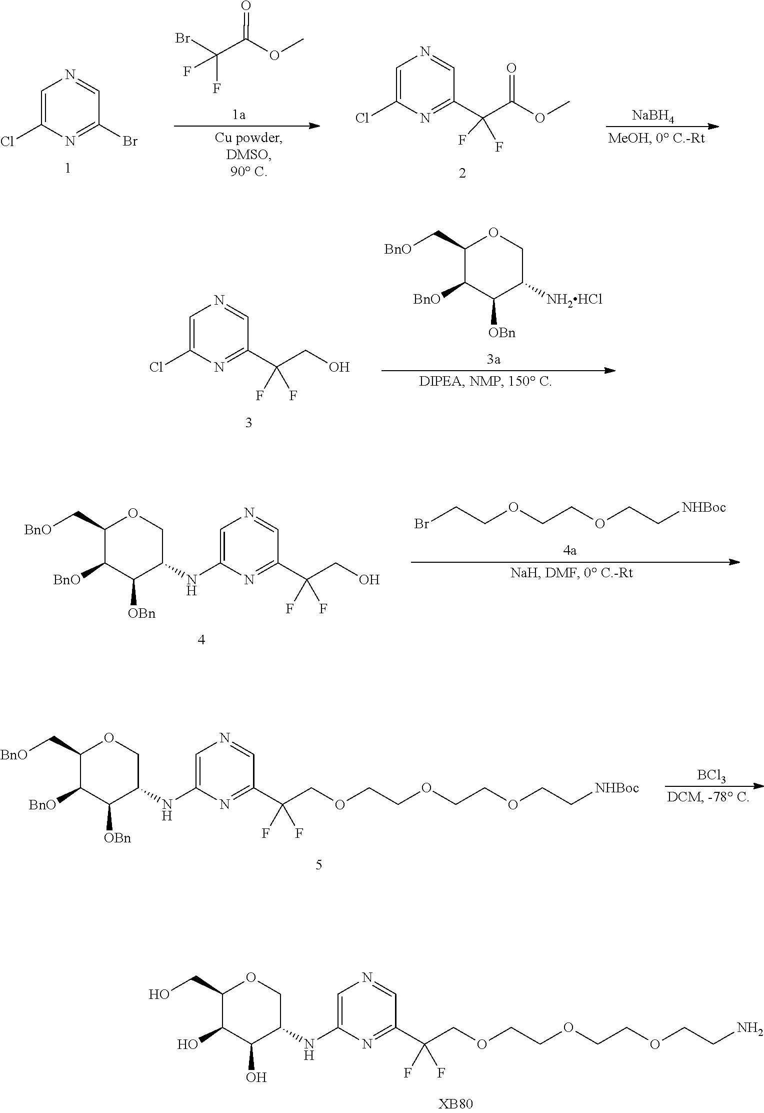

### Synthesis of methyl 2-(6-chloropyrazin-2-yl)-2,2-difluoroacetate (2)

To a stirred suspension of copper (1.97 g, 3 eq., 31 mmol) in dimethyl sulfoxide (8 mL), methyl 2-bromo-2,2-difluoroacetate (1a, 3.7 mL, 3 eq., 31 mmol) was added at room temperature under inert atmosphere. After 30 minutes, 2-bromo-6-chloropyrazine (1, 2 g, 1 eq., 10.3 mmol) was added and heated at 90° C. for 3 h. After completion of reaction (monitored by TLC), the reaction mixture was quenched with saturated aqueous ammonium chloride solution and extracted with ethyl acetate (3×25 mL). The combined organic phase was washed with brine, dried with anhydrous sodium sulphate, filtered, and concentrated under reduced pressure to afford methyl 2-(6-chloropyrazin-2-yl)-2,2-difluoroacetate (2.5 g, crude) as dark brown liquid. ¹H NMR (400 MHz, DMSO-d₆) for crude compound: δ 9.15 (s, 1H), 9.11 (s, 1H), 3.89 (s, 3H).

### Synthesis of 2-(6-chloropyrazin-2-yl)-2,2-difluoroethan-1-ol (3)

To a stirred solution of methyl 2-(6-chloropyrazin-2-yl)-2,2-difluoroacetate (2, 2.5 g, crude) in methanol (20 mL), sodium borohydride (1.2 g, 3 eq., 33 mmol) was added portion wise for 10 min at 0° C. After stirring 30 minutes at room temperature, the reaction mixture was quenched in ice cold water and organic part was extracted with dichloromethane (3×20 mL). Combined dichloromethane layer was dried over anhydrous sodium sulphate and concentrated under reduced pressure. The crude was purified by combi flash column chromatography using ethyl acetate/heptane (0-30% gradient) as eluent to afford 2-(6-chloropyrazin-2-yl)-2,2-difluoroethan-1-ol as colourless liquid. Yield: 0.8 g, 39% over two steps. ¹H NMR (400 MHz, DMSO-d₆): δ 9.02 (s, 1H), 8.95 (s, 1H), 5.73 (t, J=6.4 Hz, 1H), 4.03-3.94 (m, 2H).

### Synthesis of 2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethan-1-ol (4)

To a solution of (3S,4R,5R,6R)-4,5-bis(benzyloxy)-6-((benzyloxy)methyl)tetrahydro-2H-pyran-3-amine-hydrochloride (3a, 0.550 g, 1 eq., 1.17 mmol) in 1-methyl-2-pyrrolidinone (4 mL) was added N,N-Diisopropylethylamine (2.16 mL, 10 eq., 11.7 mmol) and stirred at room temperature for 10 minutes. After that, 2-(6-chloropyrazin-2-yl)-2,2-difluoroethan-1-ol (3, 0.228 g, 1 eq., 1.17 mmol) was added and heated the reaction mixture at 150° C. for 48 h. After completion (monitored by LCMS), reaction mixture was concentrated under reduced pressure to give crude which was purified by combi flash column chromatography using ethyl acetate/heptane as eluting system to afford 2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethan-1-ol (4) as yellow dense liquid. Yield: 0.37 g, 53%. LCMS: m/z 592.44 [M+H].

### Synthesis of tert-butyl (2-(2-(2-(2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethoxy)ethoxy)ethoxy)ethyl)carbamate (5)

To a stirred solution of 2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethan-1-ol (4, 0.37 g, 1 eq., 0.625 mmol) in N,N-dimethylformamide (3 mL), sodium hydride (0.025 g, 1 eq., 625 μmol) was added and stirred for 5 minutes at 0° C. temperature. After that, tert-butyl (2-(2-(2-bromoethoxy)ethoxy)ethyl)carbamate (4a, 0.195 g, 1 eq., 0.625 mmol) was added. The reaction mixture was stirred at room temperature for 1 h. After completion (monitored by TLC), ice-cold water (10 mL) was added to the reaction mixture. Organic part was extracted with ethyl acetate (3×10 mL), combined and dried over anhydrous sodium sulphate. Then, solvent was evaporated under reduced pressure to give crude which was purified by combi flash column chromatography using 60% ethyl acetate/heptane as eluting system to afford tert-butyl (2-(2-(2-(2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethoxy)ethoxy)ethoxy)ethyl)carbamate (5) as brown dense liquid. Yield: 0.23 g, 45.0%. LCMS: m/z 823.05 [M+H].

### Synthesis of (2R,3R,4R,5S)-5-((6-(2-(2-(2-(2-aminoethoxy)ethoxy)ethoxy)-1,1-difluoroethyl)pyrazin-2-yl)amino)-2-(hydroxymethyl)tetrahydro-2H-pyran-3,4-diol (XB80)

To a stirred solution of tert-butyl (2-(2-(2-(2-(6-(((3S,4R,5R,6R)-4,5-bis(benzyloxy)-6-((benzyloxy)methyl)tetrahydro-2H-pyran-3-yl)amino)pyrazin-2-yl)-2,2-difluoroethoxy)ethoxy)ethoxy)ethyl)carbamate (5, 0.43 g, 1 eq., 0.522 mmol) in dichloromethane (4 mL) was added trichloroborane (7.4 mL, 20 eq., 7.31 mmol, 1M solution) solution in dichloromethane at −78° C. dropwise and stirred at the same temperature for 3 h. After completion (monitored by LCMS), reaction mixture was quenched with methanol and concentrated under reduced pressure to afford crude which was purified by RP prep-HPLC (15-20% acetonitrile in water with 0.1% TFA). Fractions containing desire product were combined and lyophilized to afford (2R,3R,4R,5S)-5-((6-(2-(2-(2-(2-aminoethoxy)ethoxy)ethoxy)-1,1-difluoroethyl)pyrazin-2-yl)amino)-2-(hydroxymethyl)tetrahydro-2H-pyran-3,4-diol (XB80) as brown sticky solid. Yield: 0.112 g, 47%. ¹H NMR (400 MHz, DMSO-d₆): δ 8.07 (s, 1H), 7.90 (s, 1H), 7.72 (brs, 3H), 7.35 (d, J=7.6 Hz, 1H), 7.78 (d, J=6.4 Hz, 1H), 4.63-4.58 (m, 2H), 4.08-3.98 (m, 3H), 3.94-3.90 (m, 1H), 3.75 (brs, 1H), 3.64-3.58 (m, 2H), 3.57-3.49 (m, 11H), 3.31-3.28 (m, 1H), 2.97-2.89 (m, 3H).

### Synthesis of (2R,3R,4R,5S)-5-((4-(2-(2-(2-aminoethoxy)ethoxy)ethoxy)pyrimidin-2-yl)amino)-2-(hydroxymethyl)tetrahydro-2H-pyran-3,4-diol) (XB96)

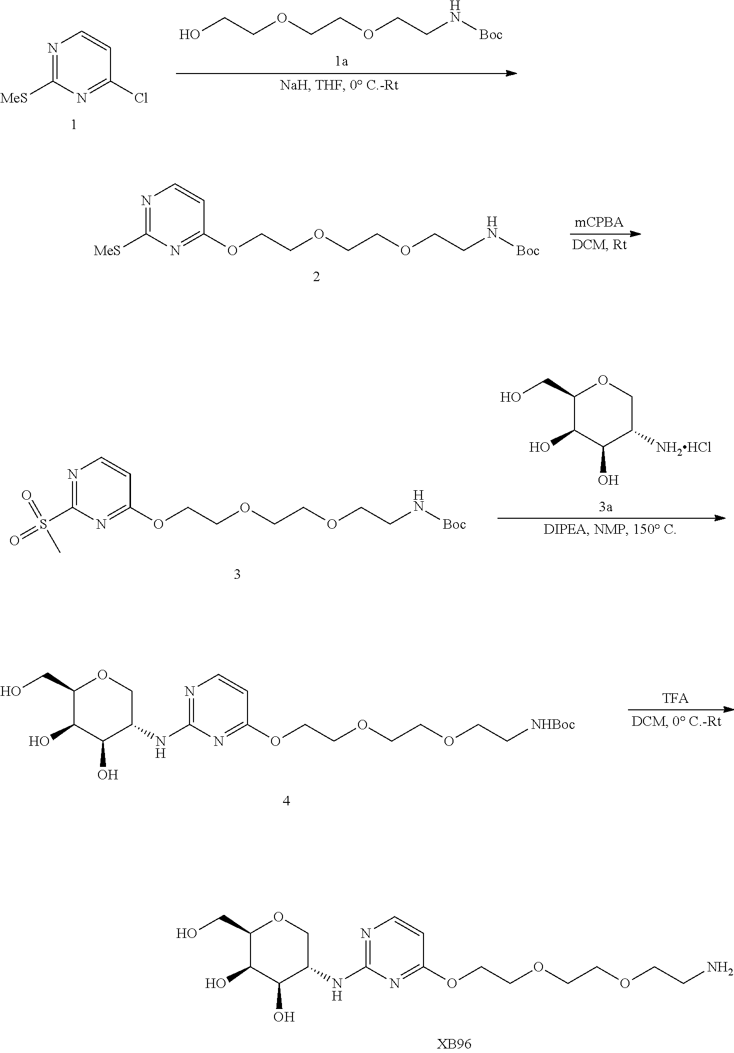

### Synthesis of tert-butyl (2-(2-(2-((2-(methylthio)pyrimidin-4-yl)oxy)ethoxy)ethoxy)ethyl)carbamate (2)

To a stirred solution of tert-butyl (2-(2-(2-hydroxyethoxy)ethoxy)ethyl)carbamate (1a, 0.706 g, 1 eq., 2.83 mmol) in tetrahydrofuran (5 mL), sodium hydride (170 mg, 1.5 eq., 4.25 mmol) was added portion wise at 0° C. under N₂ atmosphere and allowed to stir at 0° C. for 30 minutes. Then, the resultant solution was added dropwise to a solution of 4-chloro-2-(methylthio)pyrimidine (1, 0.5 g, 1 eq., 2.83 mmol) in tetrahydrofuran (2.5 mL) under N₂ atmosphere and allowed to stir for 20 minutes at 0° C. After completion (monitored by TLC), the reaction was quenched by the addition of ice-cold water, extracted with ethyl acetate, washed with brine, dried over anhydrous sodium sulfate and concentrated to get crude. Crude residue was purified by silica gel flash chromatography (eluent: 30% ethyl acetate in hexane) to afforded tert-butyl (2-(2-(2-((2-(methylthio)pyrimidin-4-yl)oxy)ethoxy)ethoxy)ethyl)carbamate (2) as sticky liquid. Yield: 1.0 g, 85.5%. LCMS: m/z 374.05 [M+H].

### Synthesis of tert-butyl (2-(2-(2-((2-(methylsulfonyl)pyrimidin-4-yl)oxy)ethoxy)ethoxy)ethyl)carbamate (3)

To a stirred solution of tert-butyl (2-(2-(2-((2-(methylthio)pyrimidin-4-yl)oxy)ethoxy)ethoxy)ethyl)carbamate (2, 1.3 g, 1 eq., 3.48 mmol) in dichloromethane (15 mL), meta-chloroperbenzoic acid (1.8 g, 2.3 eq., 7.83 mmol) was added portion wise at 0° C. under N₂ atmosphere and allowed to stir at room temperature for 12 h. After completion (monitored by LCMS), water (20 mL) was added and extracted with dichloromethane (3×15 mL), washed with sodium bicarbonate solution, dried over anhydrous sodium sulfate and concentrated under reduced pressure to get crude residue which was purified by silica gel flash chromatography (eluent: 40% ethyl acetate in hexane) to give tert-butyl (2-(2-(2-((2-(methylsulfonyl)pyrimidin-4-yl)oxy)ethoxy)ethoxy)ethyl)carbamate (3) as white solid. Yield: 0.80 g, 56.68%. LCMS: m/z 406.6 [M+H].

### Synthesis of tert-butyl (2-(2-(2-((2-(methylsulfonyl)pyrimidin-4-yl)oxy)ethoxy)ethoxy)ethyl)carbamate (4)

To a stirred solution of tert-butyl (2-(2-(2-((2-(methylsulfonyl)pyrimidin-4-yl)oxy)ethoxy)ethoxy)ethyl)carbamate (3, 0.4 g, 1 eq., 0.987 mmol) and (2R,3R,4R,5S)-5-amino-2-(hydroxymethyl)tetrahydro-2H-pyran-3,4-diol hydrochloride (3a, 0.236 g, 1.2 eq., 1.18 mmol) in N-Methyl-2-pyrrolidone (4 mL), N,N-Diisopropylethylamine (1.72 mL, 10 eq., 9.87 mmol) was added at 0° C. and heated at 150° C. for 12 h. After completion (monitored by LCMS), reaction mixture was poured into water and washed with dichloromethane. The aqueous part was lyophilized to give crude tert-butyl (2-(2-(2-((2-(((3S,4R,5R,6R)-4,5-dihydroxy-6-(hydroxymethyl)tetrahydro-2H-pyran-3-yl)amino)pyrimidin-4-yl)oxy)ethoxy)ethoxy)ethyl)carbamate, which was directly forwarded to next step. Yield: 0.60 g (Crude). LCMS: m/z 489.10 [M+H].

### Synthesis of (2R,3R,4R,5S)-5-((4-(2-(2-(2-aminoethoxy)ethoxy)ethoxy)pyrimidin-2-yl)amino)-2-(hydroxymethyl)tetrahydro-2H-pyran-3,4-diol (XB96)

To a stirred solution of tert-butyl (2-(2-(2-((2-(((3S,4R,5R,6R)-4,5-dihydroxy-6-(hydroxymethyl)tetrahydro-2H-pyran-3-yl)amino)pyrimidin-4-yl)oxy)ethoxy)ethoxy)ethyl)carbamate (0.460 g, 1 eq., 941.58 mmol) in dichloromethane (3 mL), trifluoroacetic acid (3 mL) was added at 0° C. and reaction mixture was stirred at room temperature for 3h. Progress of reaction was monitored by LCMS. After completion, reaction mixture was concentrated under reduced pressure to give crude compound was purified by RP prep-HPLC (20-25% acetonitrile in water with 0.1% TFA). Fractions containing the desired product were combined and lyophilized to afford (2R,3R,4R,5S)-5-((4-(2-(2-(2-aminoethoxy)ethoxy)ethoxy)pyrimidin-2-yl)amino)-2-(hydroxymethyl)tetrahydro-2H-pyran-3,4-diol (XB96) as white solid. Yield: 0.15 g, 33.0%; LCMS-MS: m/z 489.20 [M+H]. ¹H NMR (400 MHz, DMSO-d₆ with D₂O exchange): δ 7.99 (brs, 1H), 6.31 (brs, 1H), 4.47 (brs, 2H), 4.13 (s, 1H), 3.74 (d, J=2.8 Hz, 3H), 3.66-3.47 (m, 10H), 3.30 (t, J=5.6 Hz, 1H), 3.09-3.04 (m, 1H), 2.93 (t, J=4.8 Hz, 2H), 1.23-1.17 (m, 2H).

### Synthesis of N-((2R,3R,4R,5R,6R)-2-((2-(2-(2-(2-aminoethoxy)ethoxy)ethoxy)ethyl)thio)-4,5-dihydroxy-6-(hydroxymethyl)tetrahydro-2H-pyran-3-yl)acetamide (XB50)

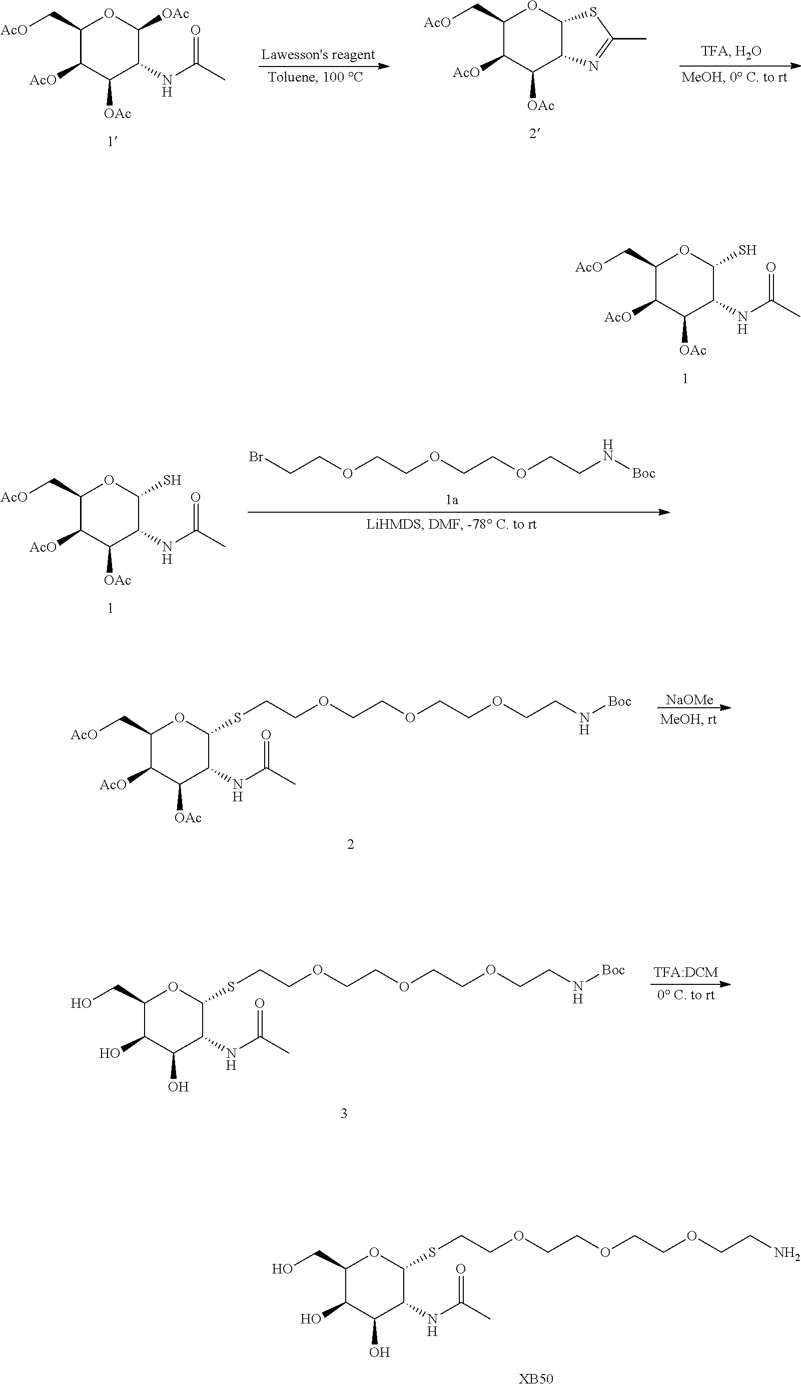

### Synthesis of (3aR,5R,6R,7R,7aR)-5-(acetoxymethyl)-2-methyl-3a,6,7,7a-tetrahydro-5H-pyrano[3,2-d]thiazole-6,7-diyl diacetate (2′)

To a solution of (2S,3R,4R,5R,6R)-3-acetamido-6-(acetoxymethyl)tetrahydro-2H-pyran-2,4,5-triyl triacetate (1′, 1.0 eq, 4.0 g, 10.3 mmol) in toluene (33.2 mL), Lawesson's reagent (0.85 eq, 3.53 g, 8.73 mmol) was added and reaction mixture was heated at 100° C. for 1.5 h. After completion, reaction mixture was cooled, water was added, neutralized with solid sodium bicarbonate and extracted with ethyl acetate. The organic layer was dried over anhydrous sodium sulfate, filtered and concentrated to get crude which was purified by column chromatography using silica gel (100-200 mesh) and 0-20% ethyl acetate in dichloromethane to afford (3aR,5R,6R,7R,7aR)-5-(acetoxymethyl)-2-methyl-3a,6,7,7a-tetrahydro-5H-pyrano[3,2-d]thiazole-6,7-diyl diacetate (2) as a light yellow viscous liquid. Yield: 2.0 g, 56.37%; LCMS m/z 346.10 [M+18]⁺.

### Synthesis of (2R,3R,4R,5R,6R)-5-acetamido-2-(acetoxymethyl)-6-mercaptotetrahydro-2H-pyran-3,4-diyl diacetate (1)

A solution of (3aR,5R,6R,7R,7aR)-5-(acetoxymethyl)-2-methyl-3a,6,7,7a-tetrahydro-5H-pyrano[3,2-d]thiazole-6,7-diyl diacetate (2′, 1.0 eq, 1.6 g, 4.63 mmol) in methanol (16 mL) and water (0.16 mL) was cooled at 0° C., trifluoroacetic acid (0.16 mL) was added and reaction mixture was stirred at room temperature for 16 h. After completion, reaction mixture was concentrated, azeotroped with toluene (2-3 times) and dried to get afford (2R,3R,4R,5R,6R)-5-acetamido-2 -(acetoxymethyl)-6-mercaptotetrahydro-2H-pyran-3,4-diyl diacetate (3) as a light yellow viscous liquid. Yield: 1.6 g (Crude); LCMS m/z 364.10 [M+H].

### Synthesis of (2R,3R,4R,5R,6R)-5-acetamido-2-(acetoxymethyl)-6-((2,2-dimethyl-4-oxo-3,8,11,14-tetraoxa-5-azahexadecan-16-yl)thio)tetrahydro-2H-pyran-3,4-diyl diacetate (2)

A solution of (2R,3R,4R,5R,6R)-5-acetamido-2-(acetoxymethyl)-6-mercaptotetrahydro-2H-pyran-3,4-diyl diacetate (1, 1.0 eq, 0.800 g, 2.2 mmol) in N,N-dimethylformamide (8 mL) was cooled at −78° C., Lithium bis(trimethylsilyl)amide (LiHMDS, 1M in tetrahydrofuran) (1.0 eq, 2.2 mL, 2.2 mmol) was added and reaction mixture was stirred at the same temperature for 1 h. Then, tert-butyl (2-(2-(2-(2-bromoethoxy)ethoxy)ethoxy)ethyl)carbamate (1a, 1.2 eq, 0.940 g, 2.64 mmol) was added and reaction mixture stirred at room temperature for 16 h. After completion, reaction mixture was concentrated to get crude which was purified by column chromatography using silica gel (100-200 mesh) and 0-5% methanol in dichloromethane to afford (2R,3R,4R,5R,6R)-5-acetamido-2-(acetoxymethyl)-6-((2,2-dimethyl-4-oxo-3,8,11,14-tetraoxa-5-azahexadecan-16-yl)thio)tetrahydro-2H-pyran-3,4-diyl diacetate (2) as a colorless semi solid. Yield: 0.600 g, 44.11%; LCMS m/z 639.05 [M+H]; H NMR (400 MHz, DMSO-d₆ with D₂O) δ 5.57 (d, J=5.2 Hz, 1H), 5.31-5.30 (m, 1H), 4.89-4.85 (m, 1H), 4.43 (t, J=6.0 Hz, 1H), 4.36-4.32 (m, 1H), 4.06-3.97 (m, 2H), 3.60-3.56 (m, 1H), 3.54-3.51 (m, 4H), 3.36 (t, J=6.0 Hz, 2H), 3.03 (t, J=5.6 Hz, 2H), 2.92 (s, 2H), 2.76 (s, 1H), 2.73-2.69 (m, 1H), 2.64-2.58 (m, 1H), 2.06 (s, 3H), 1.96-1.94 (m, 5H), 1.87 (s, 3H), 1.79 (s, 3H), 1.33 (s, 9H).

### Synthesis of tert-butyl (2-(2-(2-(2-(((2R,3R,4R,5R,6R)-3-acetamido-4,5-dihydroxy-6-(hydroxymethyl)tetrahydro-2H-pyran-2-yl)thio)ethoxy)ethoxy)ethoxy)ethyl)carbamate (3)

A solution of (2R,3R,4R,5R,6R)-5-acetamido-2-(acetoxymethyl)-6-((2,2-dimethyl-4-oxo-3,8,11,14-tetraoxa-5-azahexadecan-16-yl)thio)tetrahydro-2H-pyran-3,4-diyl diacetate (2, 1.0 eq, 0.450 g, 0.705 mmol) in methanol (5 mL) was cooled at 0° C., sodium methoxide (25% solution in methanol) (2.0 eq, 0.33 mL, 1.41 mmol) was added and reaction mixture was stirred at room temperature for 3 h. After completion, reaction mixture was neutralized with Dowex 50WX8 hydrogen form (200-400 mesh) and filtered through sintered funnel (without celite). The filtrate was concentrated, washed with diethyl ether and dried to afford tert-butyl (2-(2-(2-(2-(((2R,3R,4R,5R,6R)-3-acetamido-4,5-dihydroxy-6-(hydroxymethyl)tetrahydro-2H-pyran-2-yl)thio)ethoxy)ethoxy)ethoxy)ethyl)carbamate (3) as an off white solid. Yield: 0.320 g, 88.64%; LCMS m/z 513.10 [M+H].

### Synthesis of N-((2R,3R,4R,5R,6R)-2-((2-(2-(2-(2-aminoethoxy)ethoxy)ethoxy)ethyl)thio)-4,5-dihydroxy-6-(hydroxymethyl)tetrahydro-2H-pyran-3-yl)acetamide (XB50)

A solution of tert-butyl (2-(2-(2-(2-(((2R,3R,4R,5R,6R)-3-acetamido-4,5-dihydroxy-6-(hydroxymethyl)tetrahydro-2H-pyran-2-yl)thio)ethoxy)ethoxy)ethoxy)ethyl)carbamate (3, 1.0 eq, 0.320 g, 0.624 mmol) in dichloromethane (1.6 mL) was cooled at 0° C., trifluoroacetic acid (1.6 mL) was added and reaction mixture was stirred at room temperature for 1 h. After completion, the reaction mixture was concentrated, azeotroped with dichloromethane (2-3 times), washed with diethyl ether (2-3 times) and purified by prep HPLC (32-50% acetonitrile in water with 0.1% trifluoroacetic acid). Fractions containing the desired product were combined and lyophilized to dryness to afford N-((2R,3R,4R,5R,6R)-2-((2-(2-(2-(2-aminoethoxy)ethoxy)ethoxy)ethyl)thio)-4,5-dihydroxy-6-(hydroxymethyl)tetrahydro-2H-pyran-3-yl)acetamide, XB50, as a light yellow viscous liquid. Yield: 0.220 g, 85.33%; LCMS m/z 413.10 [M+H]; ¹H NMR (400 MHz, DMSO-d6) δ 7.79-7.73 (m, 3H), 5.43 (d, J=5.6 Hz, 1H), 4.67-4.60 (m, 3H), 4.18-4.12 (m, 1H), 3.88 (t, J=5.6 Hz, 1H), 3.73 (bs, 1H), 3.60-3.45 (m, 15H), 2.98-2.97 (m, 2H), 2.70-2.64 (m, 1H), 2.57-2.54 (m, 1H), 1.81 (s, 3H).

### Synthesis of N-((2R,3R,4R,5R,6R)-4,5-dihydroxy-6-(hydroxymethyl)-2-(5-(2-(2-(methylamino)ethoxy)ethoxy)pentyl)tetrahydro-2H-pyran-3-yl)acetamide (XB89)

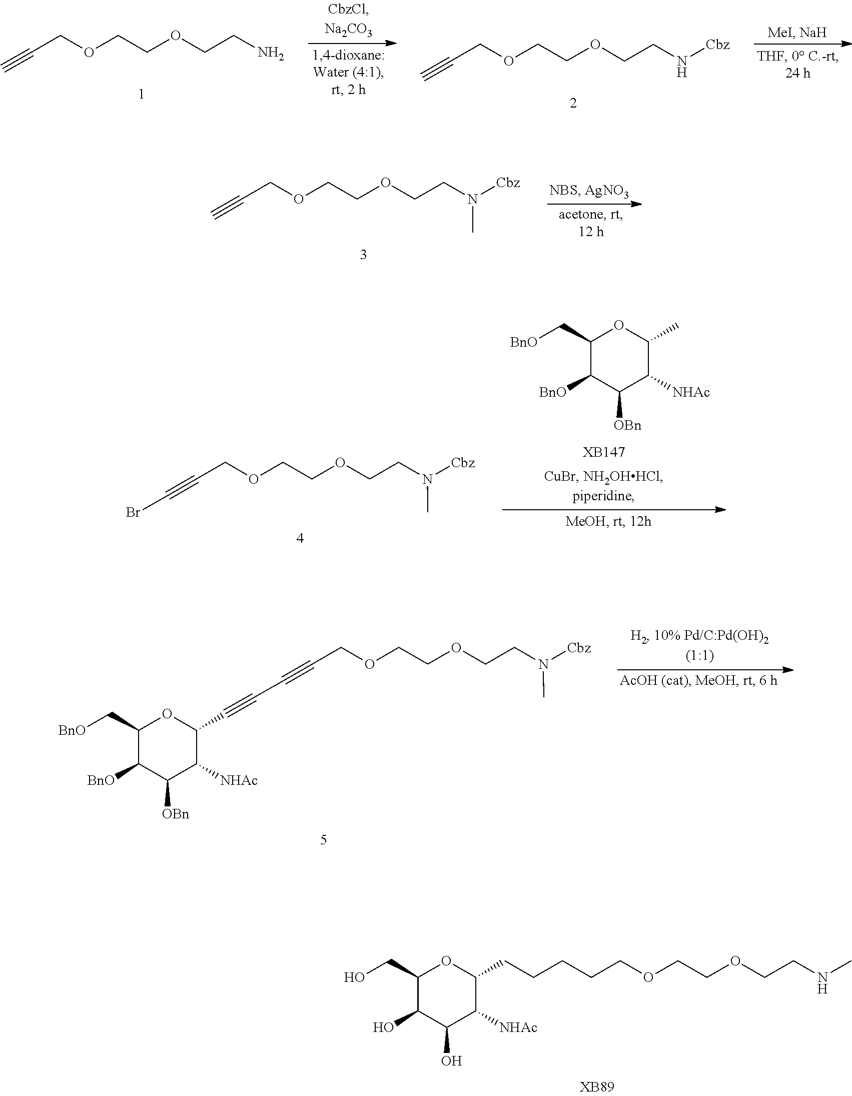

### Synthesis of benzyl (2-(2-(prop-2-yn-1-yloxy)ethoxy)ethyl)carbamate (2)

To a solution of 2-(2-(prop-2-yn-1-yloxy)ethoxy)ethan-1-amine (1, 1.50 g, 1.0 eq., 10.5 mmol) in mixture of 1,4-dioxane:water (4:1 v/v; 10 mL) was added sodium carbonate (1.67 g, 1.5 eq., 15.7 mmol) and the mixture was cooled to 0° C. To this, benzyl chloroformate (7.66 g, 1.5 eq., 15.7 mmol) was added and the reaction mixture was stirred at room temperature for 2 h. After completion, the reaction mixture was diluted by adding water and extracted with ethyl acetate. The organic layer was dried over anhydrous sodium sulphate, filtered and concentrated under reduced pressure to afford crude. Crude was triturated with pentane to afford benzyl (2-(2-(prop-2-yn-1 -yloxy)ethoxy)ethyl)carbamate (2) as colourless liquid. Yield: 2.10 g, 72.28%; LCMS: m/z 278.0 [M+H].

### Synthesis of benzyl methyl(2-(2-(prop-2-yn-1-yloxy)ethoxy)ethyl)carbamate (3)

A solution of benzyl (2-(2-(prop-2-yn-1-yloxy)ethoxy)ethyl)carbamate (2, 2.10 g, 1.0 eq., 7.57 mmol) in tetrahydrofuran (20 mL) was cooled to 0° C. Then sodium hydride (0.363 g, 1.2 eq., 9.10 mmol, 60% in oil) was added and the reaction mixture was stirred at 0° C. for 20 mins. Methyl iodide (0.566 mL, 1.20 eq., 9.10 mmol) was added and the reaction mixture was allowed to warm to room temperature and stirred for 4 h. After completion, the mixture was dried, diluted with water and extracted with ethyl acetate. The combined organic layer was dried over anhydrous sodium sulphate, concentrated under reduced pressure to get crude, which was purified by silica gel flash column chromatography using 20-40% ethyl acetate in heptane as eluent to afford benzyl methyl(2-(2-(prop-2-yn-1-yloxy)ethoxy)ethyl)carbamate (3) as a brown liquid. Yield: 1.50 g, 68.0%; LCMS: m/z 292.00 [M+H].

### Synthesis of benzyl (2-(2-((3-bromoprop-2-yn-1-yl)oxy)ethoxy)ethyl)(methyl)carbamate (4)

Benzyl methyl(2-(2-(prop-2-yn-1-yloxy)ethoxy)ethyl)carbamate (3, 1.50 g, 1.0 eq., 5.15 mmol) was dissolved in acetone (20 mL). To this, 1-bromo-2,5-pyrrolidinedione (1.01 g, 1.1 eq., 5.66 mmol) and silver nitrate (87.5 mg, 0.1 eq., 0.515 mmol) were added. The resultant reaction mixture was stirred at room temperature for 12 h. After completion, the reaction mixture was concentrated to remove acetone; and then diluted with ethyl acetate and water. The aqueous layer was extracted with ethyl acetate and the combined organic layer was dried over anhydrous sodium sulphate, filtered, and concentrated to obtained crude which was purified by silica gel flash column chromatography using 30-40% ethyl acetate in heptane as eluent to afford benzyl (2-(2-((3-bromoprop-2-yn-1-yl)oxy)ethoxy)ethyl)(methyl)carbamate (4) as viscous oil. Yield: 0.740 g, 38.88%; LCMS: m/z 369.95 [M+H].

### Synthesis of benzyl (2-(2-((5-((2R,3S,4R,5R,6R)-3-acetamido-4,5-bis(benzyloxy)-6-((benzyloxy)methyl)tetrahydro-2H-pyran-2-yl)penta-2,4-diyn-1-yl)oxy)ethoxy)ethyl)(methyl)carbamate (5)

A solution containing N-((2R,3S,4R,5S,6R)-4,5-bis(benzyloxy)-6-ethyl-2-ethynyltetrahydro-2H-pyran-3-yl)acetamide (XB147, 0.80 g, 1.6 mmol), piperidine (0.341 g, 2.5 eq., 4 mmol), copper(I)bromide (0.023 g, 0.1 eq., 160 μmol) and hydroxylamine hydrochloride (0.0223 g, 0.2 eq., 0.320 mmol) in methanol (9 mL) was degassed under N₂ atmosphere at room temperature. To this, a degassed solution of benzyl (2-(2-((3-bromoprop-2-yn-1-yl)oxy)ethoxy)ethyl)(methyl)carbamate (4, 0.711 g, 1.20 eq., 1.92 mmol) in methanol (9 mL) was added in portion over a period of 15 mins. The resultant reaction mixture was stirred at room temperature for 12 h. After completion, methanol was removed under reduced pressure and the crude product obtained was poured into ice cold water (20 mL) and extracted with ethyl acetate. The organic layer was washed with saturated brine solution (20 mL) and then dried over anhydrous sodium sulfate and concentrated under reduced pressure to obtained crude which was purified by silica gel flash column chromatography using 10-40% ethyl acetate in heptane as an eluent to afford benzyl (2-(2-((5-((2R,3S,4R,5R,6R)-3-acetamido-4,5-bis(benzyloxy)-6-((benzyloxy)methyl)tetrahydro-2H-pyran-2-yl)penta-2,4-diyn-1-yl)oxy)ethoxy)ethyl)(methyl)carbamate (5) as an off-white solid. Yield: 0.700 g, 63.30%; LCMS: m/z 789.05 [M+H].

### Synthesis of N-((2R,3R,4R,5R,6R)-4,5-dihydroxy-6-(hydroxymethyl)-2-(5-(2-(2-(methylamino)ethoxy)ethoxy)pentyl)tetrahydro-2H-pyran-3-yl)acetamide (XB89)

To a stirred solution of benzyl (2-(2-((5-((2R,3S,4R,5R,6R)-3-acetamido-4,5-bis(benzyloxy)-6-((benzyloxy)methyl)tetrahydro-2H-pyran-2-yl)penta-2,4-diyn-1-yl)oxy)ethoxy)ethyl)(methyl)carbamate (5, 0.570 g, 1.0 eq., 0.722 mmol) in methanol (10 mL) and acetic acid (1.5 mL), 10% Pd/C (0.275 g) and 20% Pd(OH)₂/C (0.275 g) were added, and the resultant reaction mixture was stirred under H₂-balloon pressure at room temperature for 6 h. After completion, the mixture was filtered through syringe filter and concentrated under reduced pressure to get crude. Crude was triturated with diethyl ether and pentane several times. Then crude was dissolved in water and lyophilized to afford N-((2R,3R,4R,5R,6R)-4,5-dihydroxy-6-(hydroxymethyl)-2-(5-(2-(2-(methylamino)ethoxy)ethoxy)pentyl)tetrahydro-2H-pyran-3-yl)acetamide (XB89) as colourless solid. Yield: 0.153 g, 53.6%, LCMS-MS m/z 393.15 [M+H].

¹H NMR (400 MHz, DMSO-d₆-D₂O) δ 3.97-3.94 (m, 1H), 3.82-3.79 (m, 1H), 3.71-3.70 (m, 2H), 3.53-3.46 (m, 10H), 3.34 (t, J=6.40 Hz, 2H), 2.77 (t, J=5.60 Hz, 2H), 2.36 (t, J=15.20 Hz, 3H), 1.81 (s, 1H), 1.48-1.43 (m, 3H), 1.31-0.16 (m, 6H).

### Synthesis of (2R,3R,4R,5S)-5-((6-(3-(2-(2-aminoethoxy)ethoxy)propyl)pyrazin-2-yl)amino)-2-(hydroxymethyl)tetrahydro-2H-pyran-3,4-diol (XB87)

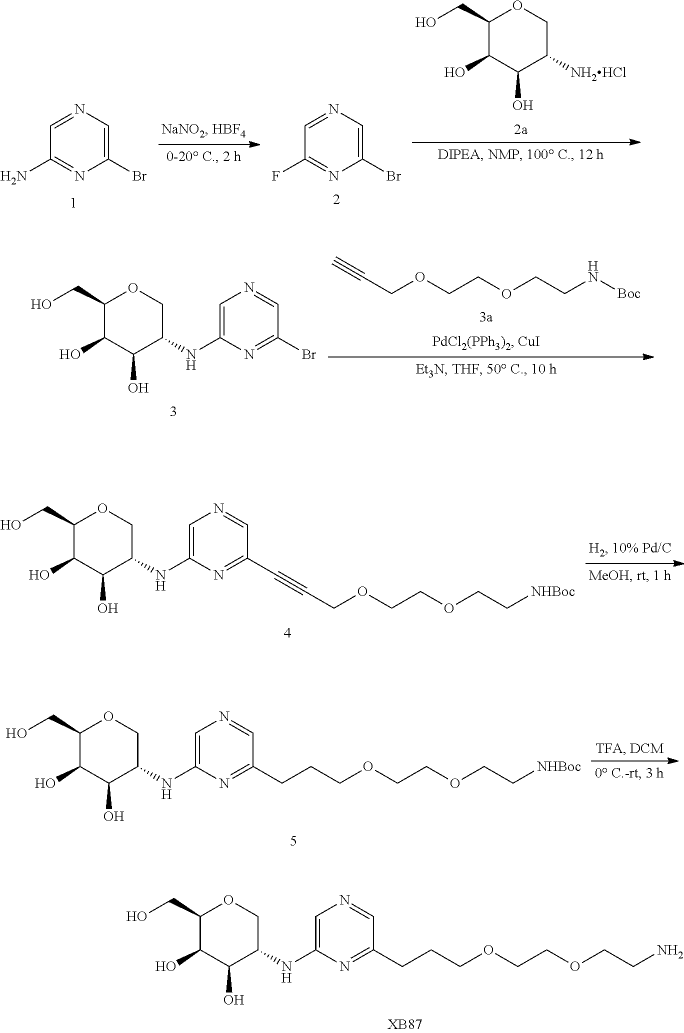

### Synthesis of 2-bromo-6-fluoropyrazine (2)

To a solution of 6-bromopyrazin-2-amine (1, 1 g, 1.0 eq. 5.75 mmol) in HBF₄ (3 mL) was added sodium nitrite (0.793 g, 2 eq., 11.5 mmol) in portions at 0° C. The mixture was stirred at 20° C. for 2 h. After completion, the reaction mixture was quenched with water (10 mL) and extracted with pentane (3×20 mL). The organic layer was dried over anhydrous sodium sulphate, filtered and concentrated via distillation to remove pentane. Desired compound 2-bromo-6-fluoropyrazine (2) was obtained as brown oil. Yield: 0.5 g, 49.16%

### Synthesis of (2R,3R,4R,5S)-5-((6-bromopyrazin-2-yl)amino)-2-(hydroxymethyl)tetrahydro-2H-pyran-3,4-diol (3)

To a stirred solution of 2-bromo-6-fluoropyrazine (2, 0.365 g, 1.0 eq., 2.06 mmol) and (2R,3R,4R,5S)-5-amino-2-(hydroxymethyl)tetrahydro-2H-pyran-3,4-diol hydrochloride (48-4, 0.494 g, 1.2 eq., 2.47 mmol) in N-Methyl-2-pyrrolidone (4 mL), N,N-Diisopropylethylamine (3.59 mL, 10 eq., 20.6 mmol) was added, and the resultant reaction mixture was allowed to stir at 100° C. for 12 h. After completion, volatiles were removed under reduced pressure to obtain crude which was purified by silica gel column chromatography using 5-20% methanol in dichloromethane to afford (2R,3R,4R,5S)-5-((6-bromopyrazin-2-yl)amino)-2-(hydroxymethyl)tetrahydro-2H-pyran-3,4-diol (3) as white semi-solid. Yield: 0.450 g, 66.79%; LCMS: m/z 320.0 [M+H].

### Synthesis of tert-butyl (2-(2-((3-(6-(((3S,4R,5R,6R)-4,5-dihydroxy-6-(hydroxymethyl)tetrahydro-2H-pyran-3-yl)amino)pyrazin-2-yl)prop-2-yn-1-yl)oxy)ethoxy)ethyl)carbamate (4)

A stirred solution of (2R,3R,4R,5S)-5-((6-bromopyrazin-2-yl)amino)-2-(hydroxymethyl)tetrahydro-2H-pyran-3,4-diol (3, 0.05 mg, 1.0 eq., 0.156 mmol) in tetrahydrofuran (0.5 mL) was purged with N₂-gas for 30 min. To this, triethylamine (0.0652 mL, 3 eq., 0.469 mmol), tert-butyl (2-(2-(prop-2-yn-1-yloxy)ethoxy)ethyl)carbamate (0.038 g, 1.0 eq., 0.156 mmol), copper iodide (0.00297 g, 0.1 eq., 0.0156 mmol) and Bis(triphenylphosphine)palladium(II) dichloride (0.0055 g, 0.05 eq., 0.0078 mmol) were added and the mixture was purged with N2-gas for another 30 minutes. The reaction mixture was stirred at 100° C. for 24 h. After completion, the reaction mixture was filtered through celite bed and rinsed with 20% methanol-dichloromethane. The filtrate was dried to obtained crude, which was purified by silica gel chromatography using 5% methanol in dichloromethane to afford tert-butyl (2-(2-((3-(6-(((3S,4R,5R,6R)-4,5-dihydroxy-6-(hydroxymethyl)tetrahydro-2H-pyran-3-yl)amino)pyrazin-2-yl)prop-2-yn-1-yl)oxy)ethoxy)ethyl)carbamate (4) as a colourless liquid. Yield: 0.011 g, 14.6%; LCMS: m/z 483.5 [M+H]

### Synthesis of tert-butyl (2-(2-(3-(6-(((3S,4R,5R,6R)-4,5-dihydroxy-6-(hydroxymethyl)tetrahydro-2H-pyran-3-yl)amino)pyrazin-2-yl)propoxy)ethoxy)ethyl)carbamate (5)

To a solution of tert-butyl (2-(2-((3-(6-(((3S,4R,5R,6R)-4,5-dihydroxy-6-(hydroxymethyl)tetrahydro-2H-pyran-3-yl)amino)pyrazin-2-yl)prop-2-yn-1-yl)oxy)ethoxy)ethyl)carbamate (4, 0.28 g, 0.580 mmol) in methanol (5 mL), 10% Pd/C (0.28 g) was added and the reaction mixture was stirred at room temperature under hydrogen gas balloon pressure for 1 h. After completion, reaction mixture was filtered through syringe filter and washed with methanol. The filtrate was concentrated and dried to afford crude tert-butyl (2-(2-(3-(6-(((3S,4R,5R,6R)-4,5-dihydroxy-6-(hydroxymethyl)tetrahydro-2H-pyran-3-yl)amino)pyrazin-2-yl)propoxy)ethoxy)ethyl)carbamate (5) as colour less liquid. Yield: 0.25 g, crude; LCMS: m/z 487.00 [M+H].

### Synthesis of (2R,3R,4R,5S)-5-((6-(3-(2-(2-aminoethoxy)ethoxy)propyl)pyrazin-2-yl)amino)-2-(hydroxymethyl)tetrahydro-2H-pyran-3,4-diol (XB87)

To a stirred solution of tert-butyl (2-(2-(3-(6-(((3S,4R,5R,6R)-4,5-dihydroxy-6-(hydroxymethyl)tetrahydro-2H-pyran-3-yl)amino)pyrazin-2-yl)propoxy)ethoxy)ethyl)carbamate (5, 0.28 g, 0.575 mol) in dichloromethane (6 mL) was added trifluoroacetic acid (1.5 mL) at 0° C. The reaction mixture was stirred at room temperature for 2 h. After completion, the reaction mixture was concentrated under vacuum to get crude which was purified by RP prep-HPLC (20-30% acetonitrile in water with 0.10% trifluoroacetic acid). Fractions containing the desired product were combined and lyophilized to dryness to afford (2R,3R,4R,5S)-5-((6-(3-(2-(2-aminoethoxy)ethoxy)propyl)pyrazin-2-yl)amino)-2-(hydroxymethyl)tetrahydro-2H-pyran-3,4-diol (XB87) as colourless semi solid. Yield: 0.107 g, 48.11%; LCMS m/z 387.10 [M+H]; H NMR (400 MHz, DMSO-d₆, D₂O exchange): δ 7.70 (s, 1H), 7.51 (s, 1H), 4.06-4.00 (m, 1H), 3.90-3.86 (m, 1H), 3.73-3.72 (d, J=2.8 Hz, 1H), 3.57-3.46 (m, 9H), 3.42-3.39 (t, J=6.4 Hz, 2H), 3.31-3.28 (t, J=6.0 Hz, 1H), 2.95-2.90 (m, 3H), 2.56-2.50 (m, 2H), 1.85-1.78 (m, 2H)

### Synthesis of N-((2S,3R,4R,5R,6R)-2-(1-(2-(2-(2-aminoethoxy)ethoxy)ethyl)-1H-pyrazol-3-yl)-4,5-dihydroxy-6-(hydroxymethyl)tetrahydro-2H-pyran-3-yl)acetamide (XB84)

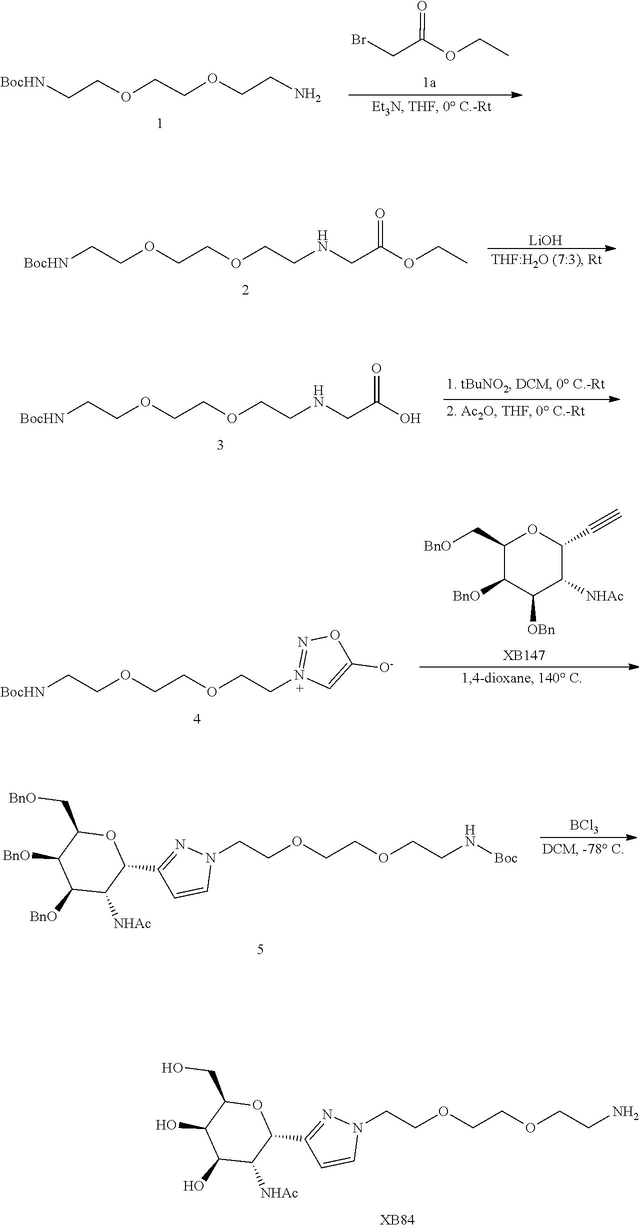

### Synthesis of ethyl (2,2-dimethyl-4-oxo-3,8,11-trioxa-5-azatridecan-13-yl)glycinate (2)

To a stirred solution of tert-butyl (2-(2-(2-aminoethoxy)ethoxy)ethyl)carbamate (1, 5.0 g, 20.1 mmol) in dry tetrahydrofuran (50 mL), were added triethylamine (8.49 mL, 3 eq., 60.4 mmol) and ethyl 2-bromoacetate (1.78 mL, 0.8 eq., 16.1 mmol) in dry tetrahydrofuran (5.0 mL) over a period of 30 min. The above suspension was stirred at room temperature for 8 h. After completion, reaction mixture was concentrated to give crude which was purified by silica gel flash column chromatography using 0-5% methanol-dichloromethane to afford ethyl (2,2-dimethyl-4-oxo-3,8,11-trioxa-5-azatridecan-13-yl)glycinate(2) as white solid. Yield: 4.6 g, 68.3%, LCMS m/z 335.2 [M+H].

### Synthesis of (2,2-dimethyl-4-oxo-3,8,11-trioxa-5-azatridecan-13-yl)glycine (3)

To a stirred solution of ethyl (2,2-dimethyl-4-oxo-3,8,11-trioxa-5-azatridecan-13-yl)glycinate (2, 4.6 g, 1.0 eq., 13.8 mmol) in tetrahydrofuran (35 mL) and water (15 mL) was added lithium hydroxide monohydrate (0.659 g, 2 eq., 27.5 mmol) at room temperature. Then reaction mixture was stirred for 3 h at room temperature. After completion, reaction mixture was concentrated under reduced pressure to get crude which was purified by RP prep HPLC (20% acetonitrile in water with 0.1% acetic acid). Fractions containing desired product were combined and lyophilized to get (2,2-dimethyl-4-oxo-3,8,11-trioxa-5-azatridecan-13-yl)glycine (3) as a white solid. Yield: 2.5 g, 59.3%. LCMS m/z 307.1 [M+H].

### Synthesis of 3-(2,2-dimethyl-4-oxo-3,8,11-trioxa-5-azatridecan-13-yl)-1,2,3-oxadiazol-3-ium-5-olate (4)

To a stirred solution of (2,2-dimethyl-4-oxo-3,8,11-trioxa-5-azatridecan-13-yl)glycine (3, 2.5 g, 8.16 mmol) in dichloromethane (15 mL) was added tert-butyl nitrite (1.26 mL, 1.1 eq., 8.98 mmol) at 0° C. Reaction mixture was then stirred at room temperature for another 1 h. After completion, reaction mixture was concentrated under reduced pressure to afford crude which was again dissolved in tetrahydrofuran (20 mL) and acetic anhydride (2.32 mL, 3 eq., 24.5 mmol) was added at 0° C. and reaction mixture was allowed to stir at room temperature for 36 h. After completion, reaction mixture was concentrated to afford crude which was purified by silica gel column chromatography using 2-3% methanol/dichloromethane as eluent to afford 3-(2,2-dimethyl-4-oxo-3,8,11-trioxa-5-azatridecan-13-yl)-1,2,3-oxadiazol-3-ium-5-olate (4) as yellow liquid. Yield: 2.0 g, 77.0% LCMS m/z 317.1 [M+H].

### Synthesis of tert-butyl (2-(2-(2-(3-((2S,3S,4R,5R,6R)-3-acetamido-4,5-bis(benzyloxy)-6-((benzyloxy)methyl)tetrahydro-2H-pyran-2-yl)-1H-pyrazol-1-yl)ethoxy)ethoxy)ethyl)carbamate (5)

Compound 3-(2,2-dimethyl-4-oxo-3,8,11-trioxa-5-azatridecan-13-yl)-1,2,3-oxadiazol-3-ium-5-olate (4, 2.54 g, 5 eq., 8.01 mmol) and N-((2R,3S,4R,5R,6R)-4,5-bis(benzyloxy)-6-((benzyloxy)methyl)-2-ethynyltetrahydro-2H-pyran-3-yl)acetamide (XB147, 0.8 g, 1.6 mmol) were taken in 1,4-dioxane (3.5 mL) and reaction mixture was stirred at 140° C. temperature for 72 h. After completion (monitored by LCMS), reaction mixture was concentrated to get crude which was purified by silica gel flash chromatography using 5% methanol/dichloromethane to afford tert-butyl (2-(2-(2-(3-((2S,3S,4R,5R,6R)-3-acetamido-4,5-bis(benzyloxy)-6-((benzyloxy)methyl)tetrahydro-2H-pyran-2-yl)-1H-pyrazol-1-yl)ethoxy)ethoxy)ethyl)carbamate (5) as sticky yellow solid. Yield: 0.48 g, 38.8% LCMS m/z 773.4 [M+H].

### Synthesis of N-((2S,3R,4R,5R,6R)-2-(1-(2-(2-(2-aminoethoxy)ethoxy)ethyl)-1H-pyrazol-3-yl)-4,5-dihydroxy-6-(hydroxymethyl)tetrahydro-2H-pyran-3-yl)acetamide (XB84)

To a stirred solution of tert-butyl (2-(2-(2-(3-((2S,3S,4R,5R,6R)-3-acetamido-4,5-bis(benzyloxy)-6-((benzyloxy)methyl)tetrahydro-2H-pyran-2-yl)-1H-pyrazol-1-yl)ethoxy)ethoxy)ethyl)carbamate (5, 0.17 g, 0.220 mmol) in dichloromethane (4.25 mL) was added trichloroborane (0.309 g, 12 eq., 2.64 mmol, 1M in dichloromethane) at −78° C. dropwise and stirred at the same temperature for 3 h. After completion of reaction, reaction mixture was quenched with methanol and concentrated under reduced pressure to afford crude which was purified by RP prep-HPLC (40% of acetonitrile in water with 0.1% TFA). Fractions containing desired product were combined and lyophilized to afford N-((2S,3R,4R,5R,6R)-2-(1-(2-(2-(2-aminoethoxy)ethoxy)ethyl)-1H-pyrazol-3-yl)-4,5-dihydroxy-6-(hydroxymethyl)tetrahydro-2H-pyran-3-yl)acetamide (XB84) as colorless foamy solid. Yield: 0.047 g, 53.1%; LCMS m/z 403.00 [M+H]. z H NMR (400 MHz, DMSO-d₆): δ 7.86 (s, 3H), 7.65 (d, J=2.40 Hz, 1H), 7.43 (d, J=8.00 Hz, 1H), 6.20 (d, J=2.00 Hz, 1H), 5.00 (d, J=5.20 Hz, 1H), 4.26-4.20 (m, 3H), 3.98 (dd, J=2.8, 9.6 Hz, 1H),), 3.98-3.96 (m, 7H), 3.61 (t, J=5.6 Hz, 1H), 3.55-3.48 (m, 9H), 2.95-2.89 (m, 2H), 1.73 (s, 3H).

### Synthesis of N-((2S,3R,4R,5R,6R)-2-(2-(2-(2-(2-aminoethoxy)ethoxy)ethoxy)ethoxy)-4,5-dihydroxy-6-(hydroxymethyl)tetrahydro-2H-pyran-3-yl)acetamide (XB52)

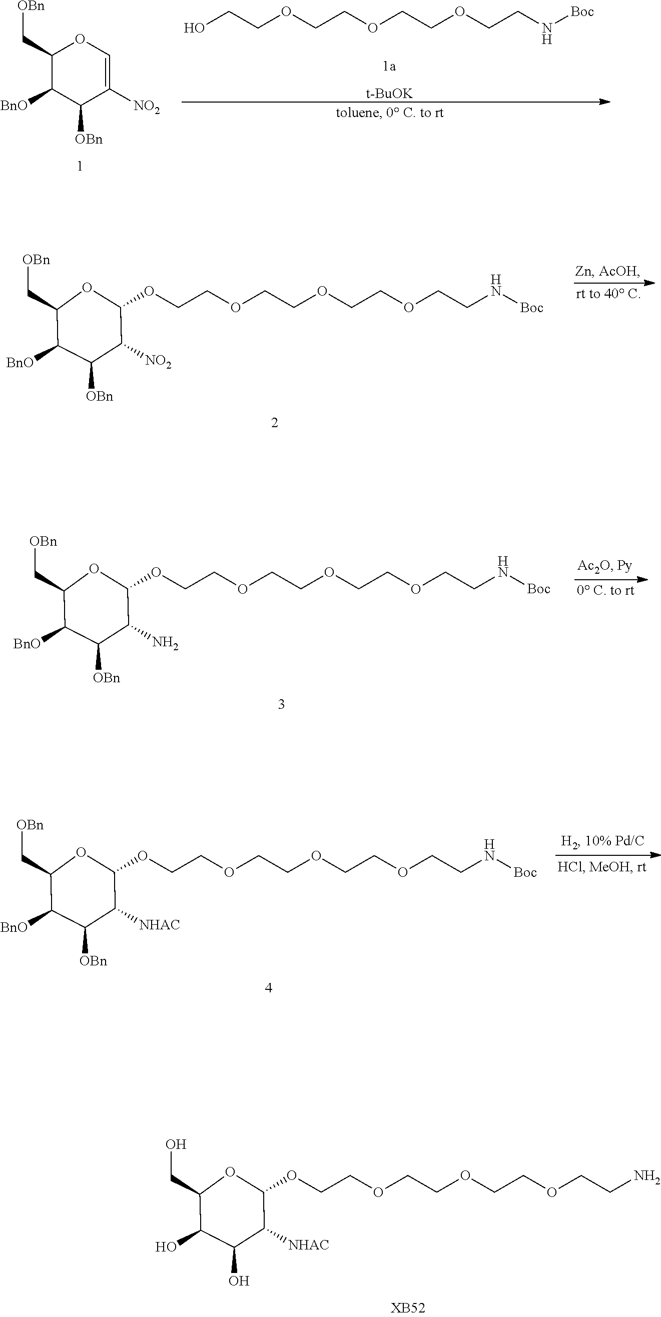

### Synthesis of tert-butyl (2-(2-(2-(2-(((2S,3R,4R,5R,6R)-4,5-bis(benzyloxy)-6-((benzyloxy)methyl)-3-nitrotetrahydro-2H-pyran-2-yl)oxy)ethoxy)ethoxy)ethoxy)ethyl)carbamate (2)

To a stirred solution of (2R,3R,4R)-3,4-bis(benzyloxy)-2-((benzyloxy)methyl)-5-nitro-3,4-dihydro-2H-pyran (1, 5.0 g, 1.0 eq., 10.8 mmol) and tert-butyl (2-(2-(2-(2-hydroxyethoxy)ethoxy)ethoxy)ethyl)carbamate (1a, 4.77 g, 1.5 eq., 16.3 mmol) in anhydrous toluene (40 mL) under Ar, activated molecular sieves (3 Å, 1.50 g) were added and the mixture stirred for 1 h at room temperature. Thereafter, t-BuOK (0.608 g, 0.5 eq., 5.42 mmol, 1 M solution in THF) was added at 0° C., and stirred for 12 h at room temperature. After completion (monitored by LCMS, & TLC), acetic acid (0.05 mL) was added to quench the reaction. Molecular sieves were filtered off and the filtrate was removed under reduced pressure to afford crude which was purified by silica gel flash column chromatography to (using 0-70% ethyl acetate in hexane) to afford tert-butyl (2-(2-(2-(2-(((2S,3R,4R,5R,6R)-4,5-bis(benzyloxy)-6-((benzyloxy)methyl)-3-nitrotetrahydro-2H-pyran-2-yl)oxy)ethoxy)ethoxy)ethoxy)ethyl)carbamate (2) as colorless syrup. Yield: 2.5 g, 30.0%; LCMS m/z 755.37 [M+H]⁺.

### Synthesis of tert-butyl (2-(2-(2-(2-(((2S,3R,4R,5R,6R)-3-amino-4,5-bis(benzyloxy)-6-((benzyloxy)methyl)tetrahydro-2H-pyran-2-yl)oxy)ethoxy)ethoxy)ethoxy)ethyl)carbamate (3)

To a solution of tert-butyl (2-(2-(2-(2-(((2S,3R,4R,5R,6R)-4,5-bis(benzyloxy)-6-((benzyloxy)methyl)-3-nitrotetrahydro-2H-pyran-2-yl)oxy)ethoxy)ethoxy)ethoxy)ethyl)carbamate (2, 2.5 g, 3.31 mmol, 1.0 eq.) in glacial acetic acid (30 mL); zinc (2.6 g, 12.0 eq., 39.7 mmol) was added and then heated at 40° C. for 3 h. After completion (monitored by TLC), the reaction mixture was diluted with methanol and was filtered through celite pad. The volatiles were evaporated out on high vacuum to yield crude tert-butyl (2-(2-(2-(2-(((2S,3R,4R,5R,6R)-3-amino-4,5-bis(benzyloxy)-6-((benzyloxy)methyl)tetrahydro-2H-pyran-2-yl)oxy)ethoxy)ethoxy)ethoxy)ethyl)carbamate (3) as syrup which was used for next step without further purification. Yield: 2.5 g (crude); LCMS m/z 725.65 [M+H].

### Synthesis of tert-butyl (2-(2-(2-(2-(((2S,3R,4R,5R,6R)-3-acetamido-4,5-bis(benzyloxy)-6-((benzyloxy)methyl)tetrahydro-2H-pyran-2-yl)oxy)ethoxy)ethoxy)ethoxy)ethyl)carbamate (4)

To a solution of tert-butyl (2-(2-(2-(2-(((2S,3R,4R,5R,6R)-3-amino-4,5-bis(benzyloxy)-6-((benzyloxy)methyl)tetrahydro-2H-pyran-2-yl)oxy)ethoxy)ethoxy)ethoxy)ethyl)carbamate (3, 2.5 g, 3.45 mmol, 1.0 eq.) in pyridine (20 mL), acetic anhydride (10 mL) was added at 0° C. and stirred the reaction mixture for 16 h at room temperature. After completion, the volatiles were evaporated under reduced pressure to get crude which was purified by silica gel flash column chromatography (40-60% ethyl acetate/hexane) to afford tert-butyl (2-(2-(2-(2-(((2S,3R,4R,5R,6R)-3-acetamido-4,5-bis(benzyloxy)-6-((benzyloxy)methyl)tetrahydro-2H-pyran-2-yl)oxy)ethoxy)ethoxy)ethoxy)ethyl)carbamate (4) as off-white semi solid Yield: 2.5 g, 94.52%; LCMS m/z 767.50 [M+H]. ¹H NMR (400 MHz, DMSO-d₆): δ 7.82 (d, J=8.8 Hz, 1H), 7.35-7.23 (m, 15H), 6.74 (t, J=5.6 Hz, 1H), 5.75 (s, 1H), 4.76-4.70 (m, 3H), 4.59-4.43 (m, 4H), 4.29-4.23 (m, 1H), 4.04 (bs, 1H), 3.94 (t, J=6.4 Hz, 1H), 3.75 (dd, J=11.2, 2.4 Hz 1H), 3.68-3.63 (m, 1H), 3.58-3.54 (m, 4H), 3.53-3.47 (m, 9H), 3.37-3.34 (m, 2H), 3.05 (q, J=6.0 Hz, 2H), 1.83 (s, 3H), 1.36 (s, 9H).

### Synthesis of N-((2S,3R,4R,5R,6R)-2-(2-(2-(2-(2-aminoethoxy)ethoxy)ethoxy)ethoxy)-4,5-dihydroxy-6-(hydroxymethyl)tetrahydro-2H-pyran-3-yl)acetamide (XB52)

To a solution of tert-butyl (2-(2-(2-(2-(((2S,3R,4R,5R,6R)-3-acetamido-4,5-bis(benzyloxy)-6-((benzyloxy)methyl)tetrahydro-2H-pyran-2-yl)oxy)ethoxy)ethoxy)ethoxy)ethyl)carbamate (4, 1.0 g, 1.0 eq., 1.3 mmol) in methanol (15 mL), 10% Pd/C (1.0 g), and concentrated HCl (0.083 mL, 2.0 eq., 2.61 mmol) were added. Then reaction mixture was stirred at room temperature under H₂ gas balloon pressure for 48 h. After completion, the reaction mixture was filtered on celite pad and washed the pad with methanol. The volatiles were evaporated in high vacuum to afford crude which was purified by prep-HPLC (25% acetonitrile in water with 0.1% TFA) to afford N-((2S,3R,4R,5R,6R)-2-(2-(2-(2-(2-aminoethoxy)ethoxy)ethoxy)ethoxy)-4,5-dihydroxy-6-(hydroxymethyl)tetrahydro-2H-pyran-3-yl)acetamide (XB52) as light brown semi solid Yield: 0.109 g, 21.09%; LCMS m/z 397.15 [M+H]H NMR (400 MHz, DMSO-d₆ with D₂O exchange) δ 4.70 (d, J=2.0 Hz, 1H), 4.01-3.98 (m, 1H), 3.77-3.72 (m, 2H), 3.63-3.44 (m, 16H), 2.95 (t, J=4.8 Hz, 2H), 1.84 (s, 3H).

### Synthesis of 3-(((2S,3R,4R,5R,6R)-3-acetamido-4,5-dihydroxy-6-(hydroxymethyl)tetrahydro-2H-pyran-2-yl)thio)-N-(5-aminopentyl)propanamide (XB54) and 3-(((2S,3R,4R,5R,6R)-3 -acetamido-4,5-dihydroxy-6-(hydroxymethyl)tetrahydro-2H-pyran-2-yl)thio)propanoic acid (XB146)

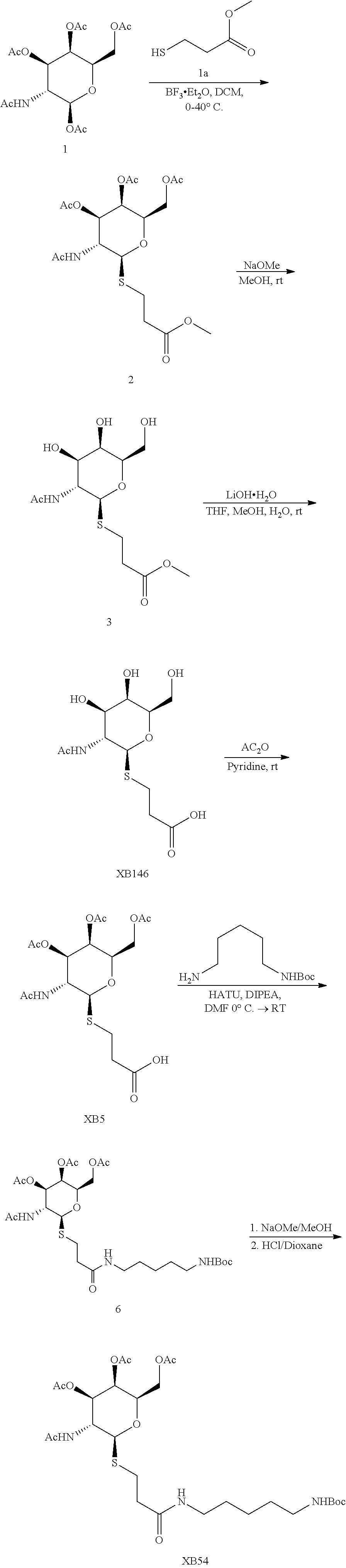

### Synthesis of (2R,3R,4R,5R,6S)-5-acetamido-2-(acetoxymethyl)-6-((3-methoxy-3-oxopropyl)thio)tetrahydro-2H-pyran-3,4-diyl diacetate (2)

A solution of (2S,3R,4R,5R,6R)-3-acetamido-6-(acetoxymethyl)tetrahydro-2H-pyran-2,4,5-triyl triacetate (1, 1.0 eq, 5.0 g, 12.8 mmol) and methyl 3-mercaptopropanoate (1a, 2.0 eq, 3.09 mL, 25.7 mmol) in dichloromethane (50 mL) was cooled at 0° C., boron trifluoride diethyl etherate (5.0 eq, 8.28 mL, 64.2 mmol) was added dropwise and reaction mixture was heated at 40° C. for 16 h. Reaction was monitored by ELSD. After completion, reaction mixture was cooled, diluted with dichloromethane, washed with saturated aqueous sodium bicarbonate solution and water. The organic layer was dried over anhydrous sodium sulfate, filtered and concentrated to get crude which was purified by column chromatography using silica gel (100-200 mesh) and 0-5% methanol in dichloromethane to afford (2R,3R,4R,5R,6S)-5-acetamido-2-(acetoxymethyl)-6-((3-methoxy-3-oxopropyl)thio)tetrahydro-2H-pyran-3,4-diyl diacetate (2) as a colorless viscous liquid. Yield: 5.2 g, 87.39%; LCMS m/z 450.1 [M+H].

### Synthesis of methyl 3-(((2S,3R,4R,5R,6R)-3-acetamido-4,5-dihydroxy-6-(hydroxymethyl)tetrahydro-2H-pyran-2-yl)thio)propanoate (3)

To a solution of (2R,3R,4R,5R,6S)-5-acetamido-2-(acetoxymethyl)-6-((3-methoxy-3-oxopropyl)thio)tetrahydro-2H-pyran-3,4-diyl diacetate (2, 1.0 eq, 4.0 g, 8.9 mmol) in methanol (40 mL), sodium methoxide (25% solution in methanol) (0.1 eq, 0.21 mL, 0.89 mmol) was added and reaction mixture was stirred at room temperature for 3 h. After completion, reaction mixture was neutralized with Dowex 50WX8 hydrogen form (200-400 mesh) and filtered through sintered funnel (without celite). The filtrate was concentrated and dried to afford methyl 3-(((2S,3R,4R,5R,6R)-3-acetamido-4,5-dihydroxy-6-(hydroxymethyl)tetrahydro-2H-pyran-2-yl)thio)propanoate (3) as an off white solid. Yield: 1.7 g, 59.0%; LCMS m/z 324.0 [M+H].

### Synthesis of 3-(((2S,3R,4R,5R,6R)-3-acetamido-4,5-dihydroxy-6-(hydroxymethyl)tetrahydro-2H-pyran-2-yl)thio)propanoic acid (XB146)

To a solution of methyl 3-(((2S,3R,4R,5R,6R)-3-acetamido-4,5-dihydroxy-6-(hydroxymethyl)tetrahydro-2H-pyran-2-yl)thio)propanoate (3, 1.0 eq, 2.0 g, 6.19 mmol) in tetrahydrofuran (18 mL), methanol (12 mL) and water (6 mL), lithium hydroxide monohydrate (2.0 eq, 0.519 g, 12.4 mmol) was added and reaction mixture was stirred at room temperature for 2 h. After completion, reaction mixture was concentrated, methanol was added, neutralized with Dowex 50WX8 hydrogen form (200-400 mesh) and filtered through sintered funnel (without celite). The filtrate was concentrated and dried to afford 3-(((2S,3R,4R,5R,6R)-3-acetamido-4,5-dihydroxy-6-(hydroxymethyl)tetrahydro-2H-pyran-2-yl)thio)propanoic acid (XB146) as an off white sticky solid. Yield: 2.4 g (Crude); LCMS m/z 310.0 [M+H].

### Synthesis of 3-(((2S,3R,4R,5R,6R)-3-acetamido-4,5-diacetoxy-6-(acetoxymethyl)tetrahydro-2H-pyran-2-yl)thio)propanoic acid (XB5)

To a solution of 3-(((2S,3R,4R,5R,6R)-3-acetamido-4,5-dihydroxy-6-(hydroxymethyl)tetrahydro-2H-pyran-2-yl)thio)propanoic acid (4, 1.0 eq, 1.1 g, 3.56 mmol) in pyridine (11 mL), acetic anhydride (10.0 eq, 3.36 mL, 35.6 mmol) was added and reaction mixture was stirred at room temperature for 16 h. After completion, reaction mixture was concentrated to get crude which was purified by column chromatography using silica gel (100-200 mesh) and 0-7% methanol in dichloromethane to afford 3-(((2S,3R,4R,5R,6R)-3-acetamido-4,5-diacetoxy-6-(acetoxymethyl)tetrahydro-2H-pyran-2-yl)thio)propanoic acid (XB5) as a colorless viscous liquid.

Yield: 1.25 g, 76.74%; LCMS m/z 436.0 [M+H]; ¹H NMR (400 MHz, DMSO-d₆) δ 12.24 (s, 1H), 7.88 (d, J=9.6 Hz, 1H), 5.27 (d, J=3.2 Hz, 1H), 4.95 (dd, J=2.4, 10.8 Hz, 1H), 4.66 (d, J=10.4 Hz, 1H), 4.10-3.96 (m, 4H), 2.85-2.79 (m, 1H), 2.74-2.69 (m, 1H), 2.58 (t, J=7.2 Hz, 2H), 2.11 (s, 3H), 2.00 (s, 3H), 1.90 (s, 3H), 1.77 (s, 3H).

A solution of tert-butyl (5-aminopentyl)carbamate (1.2 eq, 166 mg, 0.821 mmol) and 3-(((2S,3R,4R,5R,6R)-3-acetamido-4,5-diacetoxy-6-(acetoxymethyl)tetrahydro-2H-pyran-2-yl)thio)propanoic acid (XB5, 1.0 eq, 302 mg, 0.694 mmol) and DIPEA (3.0 eq, 0.36 mL, 2.08 mmol) in DMF (3.5 mL) was cooled in an ice bath before adding HATU (1.2 eq, 316 mg, 0.832 mmol) then removing the ice bath. After 45 minutes, the reaction was diluted with water (10 mL) and brine (10 mL) and the products were extracted with EtOAc (2×10 mL). The partitioned aqueous layer was washed with EtOAc (5 mL) and the combined organic layer was dried over Na2S04 and filtered. The filtrate was concentrated under reduced pressure to give crude material that was adsorbed to silica gel for purification by column chromatography (50-100% EtOAc in hexanes) to give (2R,3R,4R,5R,6S)-5-acetamido-2-(acetoxymethyl)-6-((3-((5-((tert-butoxycarbonyl)amino)pentyl)amino)-3-oxopropyl)thio)tetrahydro-2H-pyran-3,4-diyl diacetate. Yield: 319 mg, 74.2%. LCMS m/z 619.95 [M+H].

A solution of [(2R,3R,4R,5R,6S)-5-acetamido-3,4-diacetoxy-6-[3-[5-(tert-butoxycarbonylamino)pentylamino]-3-oxo-propyl]sulfanyl-tetrahydropyran-2-yl]methyl acetate (1.0 eq, 188 mg, 0.303 mmol) in methanol (1.5 mL) was treated with 25% w/w sodium methoxide in methanol (4.0 eq, 0.28 mL, 1.21 mmol). After 1 h, the reaction was cooled in an ice bath, neutralized with 4M HCl in dioxane (4.0 eq, 0.303 mL, 1.21 mmol) then concentrated under reduced pressure to give crude material. The residue was dissolved in 1:1 MeOH/DCM (3 mL) then treated with 4M HCl in dioxane (4.0 eq, 0.303 mL, 1.21 mmol). After 1 h, the reaction was concentrated under reduced pressure. The residue was dissolved in concentrated NH₄OH then purified by reversed-phase HPLC (3-30% acetonitrile in water w/10 mM NH₄OH) then lyophilized to give 3-(((2S,3R,4R,5R,6R)-3-acetamido-4,5-dihydroxy-6-(hydroxymethyl)tetrahydro-2H-pyran-2-yl)thio)-N-(5-aminopentyl)propenamide (XB54). Yield: 102 mg, 85.5%. LCMS m/z 394.2 [M+H].

### Synthesis of N-((2R,3R,4R,5R,6R)-2-((1-(2-(2-(2-(2-aminoethoxy)ethoxy)ethoxy)ethyl)-1H-1,2,3-triazol-4-yl)methyl)-4,5-dihydroxy-6-(hydroxymethyl)tetrahydro-2H-pyran-3-yl)acetamide trifluoroacetic acid (XB46)

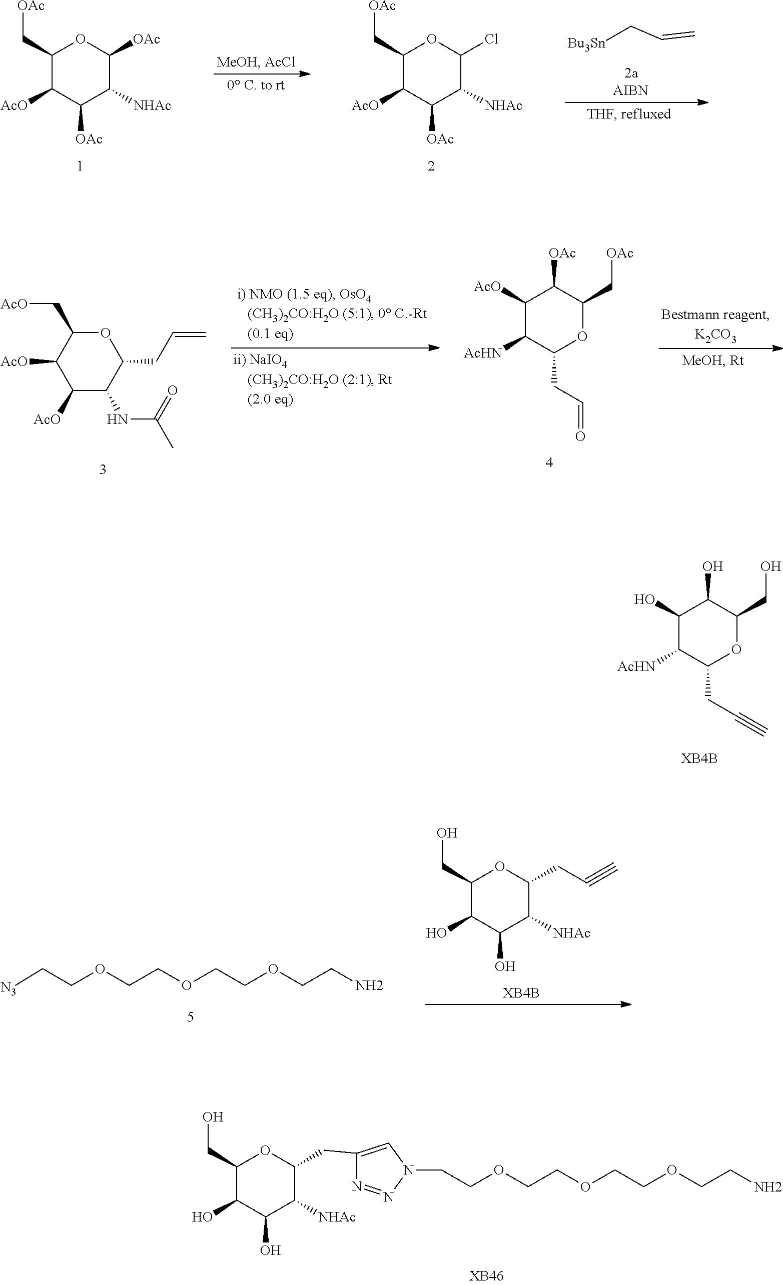

A solution of (2S,3R,4R,5R,6R)-3-acetamido-6-(acetoxymethyl)tetrahydro-2H-pyran-2,4,5-triyl triacetate (1, 1.0 eq, 20.0 g, 51.4 mmol) in acetyl chloride (60 mL) was cooled at 0° C., methanol (2.4 eq, 4.98 mL, 123.3 mmol) was added and reaction mixture was stirred at room temperature for 48 h. After completion, the reaction mixture was concentrated, washed with 10% diethyl ether in ethyl acetate and dried to afford (2R,3R,4R,5R)-5-acetamido-2-(acetoxymethyl)-6-chlorotetrahydro-2H-pyran-3,4-diyl diacetate (2) as a white solid. Yield: 16.0 g, 85.16%; ¹H NMR (400 MHz, CDCl₃) δ 6.08 (d, J=2.0 Hz, 1H), 5.36-5.31 (m, 2H), 5.26-5.25 (m, 1H), 5.29-5.25 (m, 1H), 4.15-4.02 (m, 3H), 2.16 (d, J=3.2 Hz, 3H), 2.09 (s, 3H), 2.07 (s, 3H), 2.00 (s, 3H).

To a stirred solution of (2R,3R,4R,5R)-5-acetamido-2-(acetoxymethyl)-6-chlorotetrahydro-2H-pyran-3,4-diyl diacetate (2, 5.0 g, 1.0 eq., 13.7 mmol) in dry tetrahydrofuran (50 mL) was added allyltributylstannane (2a, 42.7 mL, 10.0 eq., 137 mmol) and azobisisobutyronitrile (AIBN) (0.673 g, 0.3 eq., 4.1 mmol). The reaction mixture was then purged with argon and refluxed for 12 h. Thereafter, volatiles were removed in vacuo, and the remaining residue was partitioned between acetonitrile and pentane. Acetonitrile layer was extracted with pentane (3 times) to remove the remaining organotin compounds and then concentrated to dryness to get crude which was purified by silica gel flash column chromatography eluting with 45-50% ethyl acetate in hexane to afford (2R,3R,4R,5S,6R)-5-acetamido-2-(acetoxymethyl)-6-allyltetrahydro-2H-pyran-3,4-diyl diacetate as sticky colorless syrup. Yield: 2.4 g, 47.0%; ELSD-MS m/z 372.21 [M+H]⁺.

To a stirred solution of (2R,3R,4R,5S,6R)-5-acetamido-2-(acetoxymethyl)-6-allyltetrahydro-2H-pyran-3,4-diyl diacetate (1.5 g, 1 eq. 4.03 mmol) in acetone:water (5:1) (15 mL) was added N-methylmorpholine-N-oxide (0.708 g, 1.5 eq, 6.04 mmol) followed by osmium tetraoxide (4.0 wt % in water, 0.274 mL, 0.1 eq, 0.0403 mmol) at 0° C., and stirred for 2 h at room temperature. After completion, the reaction mixture was extracted with ethyl acetate. The organic part was then dried over anhydrous sodium sulphate, filtered, and concentrated to give crude product which was again dissolved in acetone:water (2:1) (15 mL), and added sodium periodate (1.55 g, 2.0 eq., 8.06 mmol), stirred for another 3 h at room temperature. Thereafter, reaction mixture was extracted with ethyl acetate, and the collected ethyl acetate was dried over anhydrous sodium sulphate, filtered, and concentrated to give crude (2R,3R,4R,5S,6R)-5-acetamido-2-(acetoxymethyl)-6-(2-oxoethyl)tetrahydro-2H-pyran-3,4-diyl diacetate (4) as colorless syrup. This was immediately used for next step without purification. Yield: 1.4 g (Crude); ELSD-MS m/z 374.19 [M+H]⁺.

To a stirred solution of (2R,3R,4R,5S,6R)-5-acetamido-2-(acetoxymethyl)-6-(2-oxoethyl)tetrahydro-2H-pyran-3,4-diyl diacetate (1.0 g, 1.00 eq, 2.68 mmol) in methanol (25.0 mL) at 0° C., were added potassium carbonate (1.11 g, 3.0 eq., 8.04 mmol), dimethyl (1-diazo-2-oxopropyl)phosphonate (1.03 g, 2.0 eq., 5.36 mmol) and stirred at room temperature for 5 h. Thereafter, volatiles were evaporated in vacuo to get crude which was purified by prep-HPLC (70% acetonitrile in water with 0.1% trifluoroacetic acid). Fractions containing the desired product were combined and lyophilized to dryness to afford N-((2R,3R,4R,5R,6R)-4,5-dihydroxy-6-(hydroxymethyl)-2-(prop-2-yn-1-yl)tetrahydro-2H-pyran-3-yl)acetamide (XB4B) as an off-white solid. Yield: 0.112 g, 17.04%. LCMS m/z 244.11 [M+H]⁺. ¹H NMR (400 MHz, DMSO-d₆ with D₂O exchange) δ 4.05-4.00 (m, 2H), 3.73 (bs, 1H), 3.58-3.45 (m, 4H), 2.60 (t, J=2.4 Hz, 1H), 2.46-2.41 (m, 1H), 2.33-2.27 (m, 1H), 1.82 (s, 3H).

### Synthesis of N-((2R,3R,4R,5R,6R)-2-((1-(2-(2-(2-(2-aminoethoxy)ethoxy)ethoxy)ethyl)-1H-1,2,3-triazol-4-yl)methyl)-4,5-dihydroxy-6-(hydroxymethyl)tetrahydro-2H-pyran-3-yl)acetamide trifluoroacetic acid (XB46)

To a solution of 2-(2-(2-(2-azidoethoxy)ethoxy)ethoxy)ethan-1-amine (5) (101.3 mg, 0.464 mmol, 1.00 eq) and N-((2R,3R,4R,5R,6R)-4,5-dihydroxy-6-(hydroxymethyl)-2-(prop-2-yn-1-yl)tetrahydro-2H-pyran-3-yl)acetamide (XB4B) (116.1 mg, 0.477 mmol, 1.03 eq) in 2 mL dimethyl sulfoxide was added tetrakis(acetonitrile)copper(I) hexafluorophosphate (187.3 mg, 0.403 mmol, 1.1 eq) as a solid in one portion. The mixture stirred under nitrogen atmosphere at ambient temperature for approximately 30 minutes, then directly purified by preparatory HPLC, eluting with 1-30% acetonitrile in water with 0.1% trifluoroacetic acid. Fractions containing the desired product were combined and lyophilized to dryness to afford trifluoroacetic acid salt of Compound XB46, as a white foam. Yield: 217 mg (81%); LCMS m/z 462.4 [M+H].

### Synthesis of XB105

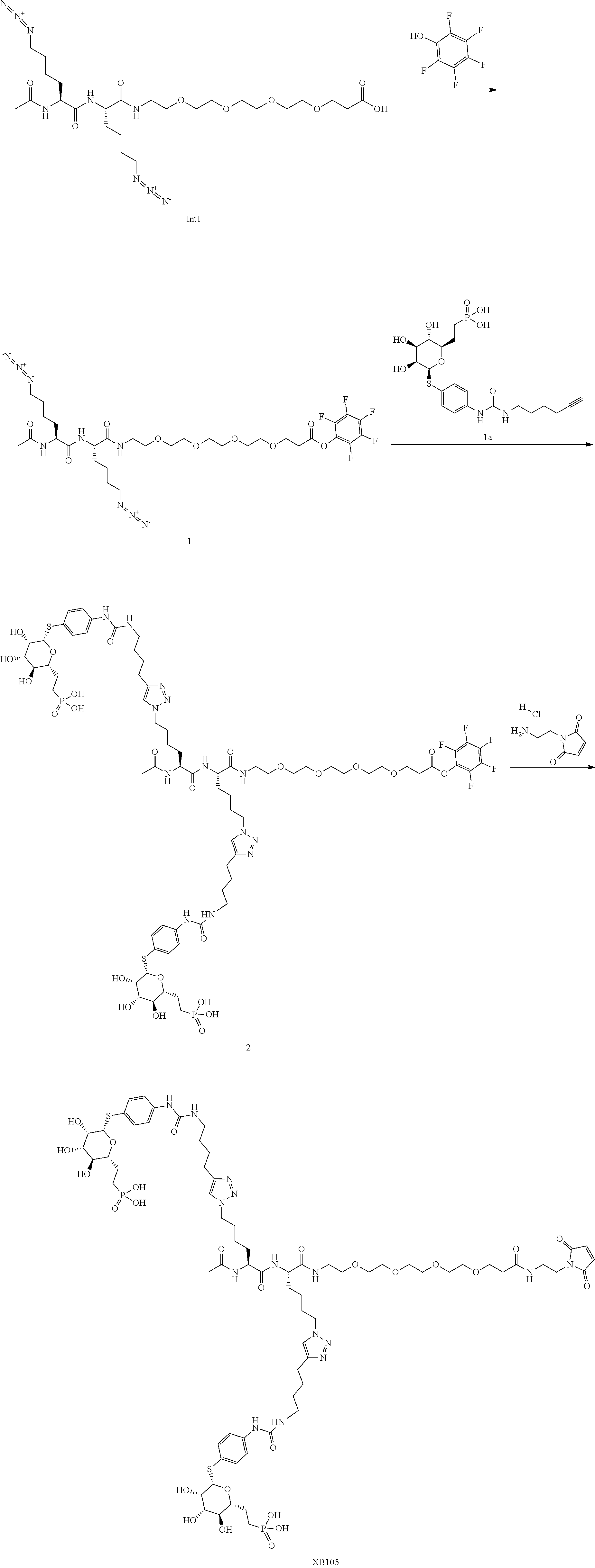

### Synthesis of perfluorophenyl (4S,7S)-4,7-bis(4-azidobutyl)-2,5,8-trioxo-12,15,18,21-tetraoxa-3,6,9-triazatetracosan-24-oate 1

A solution of Int1 (39 mg, 0.063 mmol, 1.0 eq.) in 1.0 mL of DCM was added 2,3,4,5,6-pentafluorophenol (30 mg, 0.16 mmol, 2.6 eq.), followed by 1,3-diisopropylcarbodiimide (30 μL, 0.19 mmol, 3.1 eq.). The solution stirred under nitrogen atmosphere at ambient temperature for 30 minutes. The reaction solution was diluted further with DCM and evaporated onto silica for chromatography dry load. The product was isolated by flash silica chromatography, eluting with 0-10% MeOH/DCM. Fractions containing desired product were combined, concentrated, and dried via high vacuum at ambient temperature to afford 1 as a white film. Yield: 26.8 mg (54%); LCMS m/z 782.2 [M+H].

### Synthesis of (2-((2R,3S,4S,5S,6S)-6-((4-(3-(4-(1-((S)-18-((S)-2-acetamido-6-(4-(4-(3-(4-(((2S,3S,4S,5S,6R)-3,4,5-trihydroxy-6-(2-phosphonoethyl)tetrahydro-2H-pyran-2-yl)thio)phenyl)ureido)butyl)-1H-1,2,3-triazol-1-yl)hexanamido)-1,17-dioxo-1-(perfluorophenoxy)-4,7,10,13-tetraoxa-16-azadocosan-22-yl)-1H-1,2,3-triazol-4-yl)butyl)ureido)phenyl)thio)-3,4,5-trihydroxytetrahydro-2H-pyran-2-yl)ethyl)phosphonic acid (2)

To a solution of 1 (12.8 mg, 0.0262 mmol, 1.0 eq.) in 0.5 mL DMSO was added (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (1a) (43.5 mg, 0.0556, 2.1 eq.), followed by cuprous; acetonitrile; hexafluorophosphate (32.0 mg, 0.0859 mmol, 3.3 eq.). The reaction solution stirred under nitrogen atmosphere at ambient temperature for 45 minutes, then was diluted with DMSO, acidified with 3× drops of neat trifluoroacetic acid (TFA), and purified by reverse phase HPLC (acetonitrile in water with 0.10% TFA modifier). Fractions containing desired product were combined and lyophilized to dryness to afford 2 as a white solid. Yield: 21.5 mg (45%); LCMS m/z 1758.0 [M+H].

### Synthesis of (2-((2R,3S,4S,5S,6S)-6-((4-(3-(4-(1-((S)-21-((S)-2-acetamido-6-(4-(4-(3-(4-(((2S,3S,4S,5S,6R)-3,4,5-trihydroxy-6-(2-phosphonoethyl)tetrahydro-2H-pyran-2-yl)thio)phenyl)ureido)butyl)-1H-1,2,3-triazol-1-yl)hexanamido)-1-(2,5-dioxo-2,5-dihydro-1H-pyrrol-1-yl)-4,20-dioxo-7,10,13,16-tetraoxa-3,19-diazapentacosan-25-yl)-1H-1,2,3-triazol-4-yl)butyl)ureido)phenyl)thio)-3,4,5-trihydroxytetrahydro-2H-pyran-2-yl)ethyl)phosphonic acid (XB105)

A solution of 2 (17.0 mg, 0.0097 mmol, 1.0 eq.) in 0.7 mL DMF was added 2-maleimidoethylamine hydrochloride (2.1 mg, 0.012 mmol, 1.2 eq.), followed by N,N-diethylethanamine (10 μL, 0.072 mmol, 7.4 eq.). The mixture stirred at ambient temperature for 10 minutes, then was diluted with 1 mL of DMSO, acidified with 3× drops of neat trifluoroacetic acid (TFA), and purified by reverse phase HPLC (acetonitrile in water with 0.1% TFA modifier). Fractions containing desired product were combined and lyophilized to dryness to afford XB105 as a white solid. Yield: 9.7 mg (58%); LCMS m/z 1714.4 [M+H].

### Synthesis of (4S,7S)-4,7-bis(4-azidobutyl)-2,5,8-trioxo-12,15,18,21-tetraoxa-3,6,9-triazatetracosan-24-oic acid (Int1)

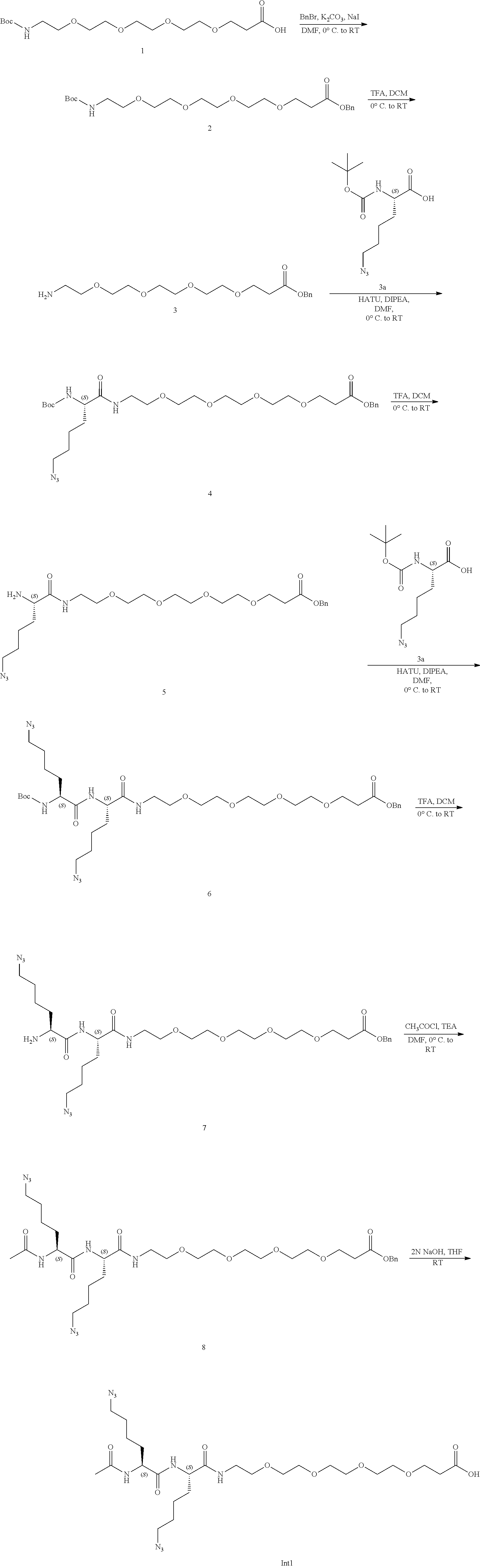

### Synthesis of benzyl 2,2-dimethyl-4-oxo-3,8,11,14,17-pentaoxa-5-azaicosan-20-oate (2)

To a solution of 2,2-dimethyl-4-oxo-3,8,11,14,17-pentaoxa-5-azaicosan-20-oic acid (1, 1.0 eq, 5.0 g, 13.7 mmol) in N,N-dimethylformamide (50 mL), potassium carbonate (3.0 eq, 5.67 g, 41.0 mmol) and sodium iodide (0.1 eq, 0.205 g, 1.37 mmol) were added, cooled at 0° C., benzyl bromide (1.5 eq, 2.51 mL, 20.5 mmol) was added and reaction mixture was stirred at room temperature for 16 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-3% methanol in dichloromethane to afford benzyl 2,2-dimethyl-4-oxo-3,8,11,14,17-pentaoxa-5-azaicosan-20-oate (2) as a colourless viscous liquid. Yield: 5.6 g, 89.65%; LCMS m/z 456.25 [M+1]⁺.

### Synthesis of benzyl 1-amino-3,6,9,12-tetraoxapentadecan-15-oate (3)

A solution of benzyl 2,2-dimethyl-4-oxo-3,8,11,14,17-pentaoxa-5-azaicosan-20-oate (2, 1.0 eq, 2.7 g, 5.93 mmol) in dichloromethane (13.5 mL) was cooled at 0° C., trifluoroacetic acid (13.5 mL) was added and reaction mixture was stirred at room temperature for 1 h. After completion, reaction mixture was concentrated, azeotroped with dichloromethane (2-3 times) and dried to afford benzyl 1-amino-3,6,9,12-tetraoxapentadecan-15-oate (3) as a light brown viscous liquid. Yield: 4.2 g (Crude); LCMS m/z 356.20 [M+1]⁺.

### Synthesis of benzyl (S)-6-(4-azidobutyl)-2,2-dimethyl-4,7-dioxo-3,11,14,17,20-pentaoxa-5,8-diazatricosan-23-oate (4)

A solution of N2-(tert-butoxycarbonyl)-N6-diazo-L-lysine (3a, 1.0 eq, 1.6 g, 5.88 mmol) and benzyl 1-amino-3,6,9,12-tetraoxapentadecan-15-oate (3, 2.0 eq, 4.18 g, 11.8 mmol) in N,N-dimethylformamide (26 mL) was cooled at 0° C., N,N diisopropylethylamine (5.0 eq, 5.12 mL, 29.4 mmol) and 1-[bis(dimethylamino)methylene]-1H-1,2,3-triazolo[4,5-b]pyridinium 3-oxide hexafluorophosphate (HATU) (1.5 eq, 3.35 g, 8.81 mmol) were added and reaction mixture was stirred at room temperature for 3 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-10% methanol in dichloromethane to afford benzyl (S)-6-(4-azidobutyl)-2,2-dimethyl-4,7-dioxo-3,11,14,17,20-pentaoxa-5,8-diazatricosan-23-oate (4) as a light brown viscous liquid. Yield: 3.0 g, 83.74%; LCMS m/z 610.10 [M+1]⁺.

### Synthesis of benzyl (S)-18-amino-22-azido-17-oxo-4,7,10,13-tetraoxa-16-azadocosanoate (5)

A solution of benzyl (S)-6-(4-azidobutyl)-2,2-dimethyl-4,7-dioxo-3,11,14,17,20-pentaoxa-5,8-diazatricosan-23-oate (4, 1.0 eq, 3.3 g, 5.41 mmol) in dichloromethane (16.5 mL) was cooled at 0° C., trifluoroacetic acid (16.5 mL) was added and reaction mixture was stirred at 0° C. for 1 h. After completion, reaction mixture was concentrated, azeotroped with dichloromethane (2-3 times), washed with diethyl ether and dried to afford benzyl (S)-18-amino-22-azido-17-oxo-4,7,10,13-tetraoxa-16-azadocosanoate (5) as a light brown viscous liquid. Yield: 1.9 g (Crude); LCMS m/z 510.20 [M+1]⁺.

### Synthesis of di-tert-butyl 3,3′-((2-((3-(tert-butoxy)-3-oxopropoxy)methyl)-2-(pent-4-ynamido)propane-1,3-diyl)bis(oxy))dipropionate (6)

A solution of N2-(tert-butoxycarbonyl)-N6-diazo-L-lysine (3a, 1.0 eq, 0.850 g, 3.12 mmol) and benzyl (S)-18-amino-22-azido-17-oxo-4,7,10,13-tetraoxa-16-azadocosanoate (5, 1.1 eq, 1.75 g, 3.43 mmol) in N,N-dimethylformamide (10 mL) was cooled at 0° C., N,N diisopropylethylamine (5.0 eq, 2.72 mL, 15.6 mmol) and 1-[bis(dimethylamino)methylene]-1H-1,2,3-triazolo[4,5-b]pyridinium 3-oxide hexafluorophosphate (HATU) (1.5 eq, 1.78 g, 4.68 mmol) were added and reaction mixture was stirred at room temperature for 3 h. After completion, water was added to reaction mixture and extracted with ethyl acetate. The organic layer was washed with water, dried over anhydrous sodium sulfate, filtered and concentrated to get crude which was purified by column chromatography using silica gel (100-200 mesh) and 0-6% methanol in dichloromethane to afford benzyl (6S,9S)-6,9-bis(4-azidobutyl)-2,2-dimethyl-4,7,10-trioxo-3,14,17,20,23-pentaoxa-5,8,11-triazahexacosan-26-oate (6) as a colourless viscous liquid. Yield: 1.68 g, 68.24%; LCMS m/z 764.10 [M+1]⁺.

### Synthesis of benzyl (18S,21S)-21-amino-25-azido-18-(4-azidobutyl)-17,20-dioxo-4,7,10,13-tetraoxa-16,19-diazapentacosanoate (7)

A solution of benzyl (6S,9S)-6,9-bis(4-azidobutyl)-2,2-dimethyl-4,7,10-trioxo-3,14,17,20,23-pentaoxa-5,8,11-triazahexacosan-26-oate (6, 1.0 eq, 1.68 g, 2.2 mmol) in dichloromethane (8.5 mL) was cooled at 0° C., trifluoroacetic acid (8.5 mL) was added and reaction mixture was stirred at 0° C. for 1 h. After completion, reaction mixture was concentrated, azeotroped with dichloromethane (2-3 times), washed with diethyl ether and dried to afford benzyl (S)-18-amino-22-azido-17-oxo-4,7,10,13-tetraoxa-16-azadocosanoate (7) as a light brown viscous liquid. Yield: 1.6 g (Crude); LCMS m/z 664.20 [M+1]⁺.

### Synthesis of benzyl (4S,7S)-4,7-bis(4-azidobutyl)-2,5,8-trioxo-12,15,18,21-tetraoxa-3,6,9-triazatetracosan-24-oate (8)

A solution of benzyl (18S,21S)-21-amino-25-azido-18-(4-azidobutyl)-17,20-dioxo-4,7,10,13-tetraoxa-16,19-diazapentacosanoate (7, 1.0 eq, 0.600 g, 0.904 mmol) in dichloromethane (6 mL) was cooled at 0° C., triethylamine (3.0 eq, 0.39 mL, 2.71 mmol) and acetyl chloride (1.5 eq, 0.096 mL, 1.36 mmol) were added and reaction mixture was stirred at room temperature for 2 h. Reaction was monitored by ELSD. After completion, reaction mixture was concentrated to get crude which was purified by column chromatography using silica gel (100-200 mesh) and 0-5% methanol in dichloromethane to afford benzyl (4S,7S)-4,7-bis(4-azidobutyl)-2,5,8-trioxo-12,15,18,21-tetraoxa-3,6,9-triazatetracosan-24-oate (8) as a light yellow viscous liquid. Yield: 0.390 g, 60.98%; LCMS m/z 706.35 [M+1]⁺.

### Synthesis of (4S,7S)-4,7-bis(4-azidobutyl)-2,5,8-trioxo-12,15,18,21-tetraoxa-3,6,9-triazatetracosan-24-oic acid (Int1)

To a solution of benzyl (4S,7S)-4,7-bis(4-azidobutyl)-2,5,8-trioxo-12,15,18,21-tetraoxa-3,6,9-triazatetracosan-24-oate (8, 1.0 eq, 0.390 g, 0.553 mmol) in tetrahydrofuran (4 mL), 2N aqueous sodium hydroxide solution (2.0 eq, 0.55 mL, 1.11 mmol) was added and reaction mixture was stirred at room temperature for 16 h. After completion, the reaction mixture was concentrated and purified by prep HPLC (23-35% acetonitrile in water with 0.05% trifluoroacetic acid). Fractions containing the desired product were combined and lyophilized to dryness to afford (4S,7S)-4,7-bis(4-azidobutyl)-2,5,8-trioxo-12,15,18,21-tetraoxa-3,6,9-triazatetracosan-24-oic acid (Int1) as a colourless semi solid. Yield: 0.186 g, 54.7%; LCMS m/z 616.30 [M+1]⁺; ¹H NMR (400 MHz, DMSO-d₆ with D₂O) δ 4.16-4.13 (m, 2H), 3.57 (t, J=6.0 Hz, 2H), 3.47-3.46 (m, 10H), 3.38 (t, J=5.6 Hz, 2H), 3.29-3.23 (m, 4H), 3.21-3.18 (in, 1H), 3.16-3.11 (m, 1H), 2.41 (t, J=6.4 Hz, 2H), 1.83 (s, 3H), 1.68-1.58 (m, 2H), 1.55-1.42 (m, 7H), 1.33-1.22 (in, 4H).

The following compounds were prepared according to the procedures disclosed above using the starting amines disclosed above via the general methods indicated.

| | | | LCMS |
|---|---|---|---|
| Compound | | Synthesis | m/z |
| No. | structure | Method | observed |
| XB91 | 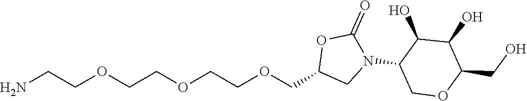 [SMILES: NCCOCCOCCOC[C@H]1CN([C@H]2CO[C@H](CO)[C@H](O)[C@@H]2O)C(=O)O1] | Provided | 395.1 [M + H] |
| XB48 | 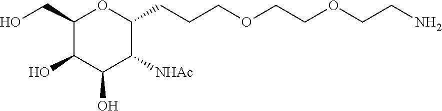 [SMILES: CC(=O)N[C@@H]1[C@@H](O)[C@@H](O)[C@@H](CO)O[C@@H]1CCCOCCOCCN] | Provided | 351.2 [M + H] |
| XB85 | 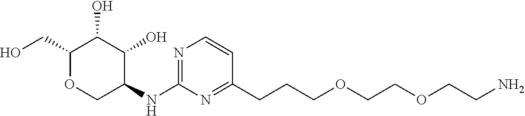 [SMILES: NCCOCCOCCCc1ccnc(N[C@H]2CO[C@H](CO)[C@H](O)[C@@H]2O)n1] | provided | 387.1 [M + H] |
| XB86 | 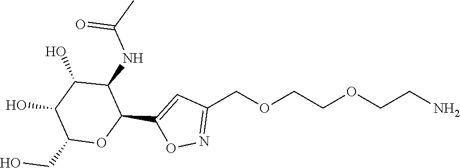 [SMILES: CC(=O)N[C@@H]1[C@@H](O)[C@@H](O)[C@@H](CO)O[C@@H]1c1cc(COCCOCCN)no1] | Provided | 390.0 [M + H] |
| XB82 | 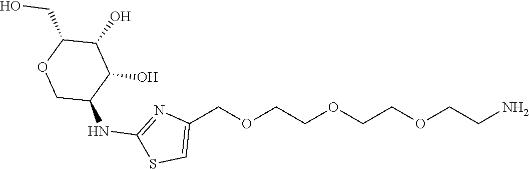 [SMILES: NCCOCCOCCOCc1csc(N[C@H]2CO[C@H](CO)[C@H](O)[C@@H]2O)n1] | provided | 408.25 [M + H] |
| XB94 | 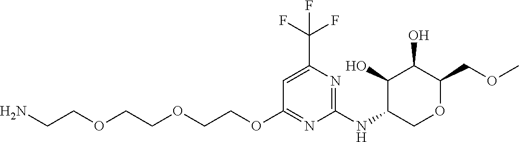 [SMILES: COC[C@H]1OC[C@H](Nc2nc(OCCOCCOCCN)cc(C(F)(F)F)n2)[C@@H](O)[C@H]1O] | provided | 471.3 [M + H] |
| XB92 | 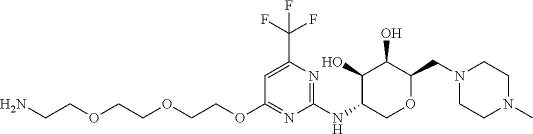 [SMILES: CN1CCN(C[C@H]2OC[C@H](Nc3nc(OCCOCCOCCN)cc(C(F)(F)F)n3)[C@@H](O)[C@H]2O)CC1] | provided | 539.2 [M + H] |
| XB97 | 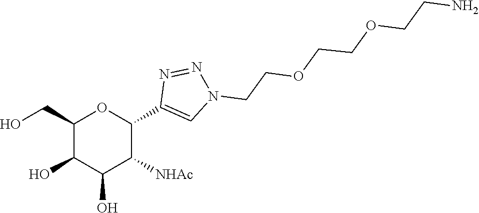 [SMILES: CC(=O)N[C@@H]1[C@@H](O)[C@@H](O)[C@@H](CO)O[C@@H]1c1cn(CCOCCOCCN)nn1] | provided | 404.3 [M + H] |
| XB103 | 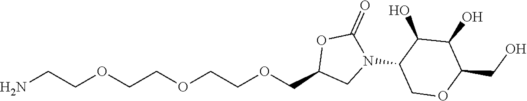 [SMILES: NCCOCCOCCOC[C@@H]1CN([C@H]2CO[C@H](CO)[C@H](O)[C@@H]2O)C(=O)O1] | analogous to XB91 using (S)-2- (chloro- methyl)- oxirane | 395.1 [M + H] |
| XB83 | 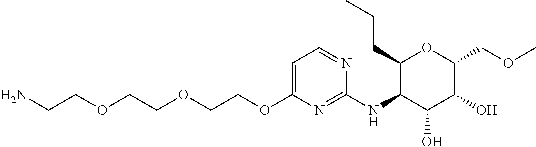 [SMILES: CCC[C@H]1O[C@H](COC)[C@H](O)[C@H](O)[C@H]1Nc1nccc(OCCOCCOCCN)n1] | Provided | 445.3 [M + H] |
| XB57 | 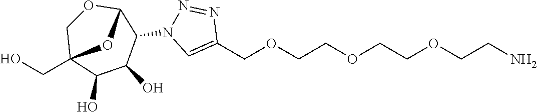 [SMILES: NCCOCCOCCOCc1cn([C@H]2[C@H]3OC[C@](CO)(O3)[C@H](O)[C@@H]2O)nn1] | Provided | 405.2 [M + H] |
| XB80 | 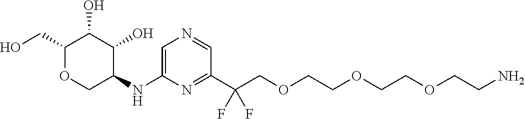 [SMILES: NCCOCCOCCOCC(F)(F)c1cncc(N[C@H]2CO[C@H](CO)[C@H](O)[C@@H]2O)n1] | Provided | 305.2 [M + H] |
| XB100 | 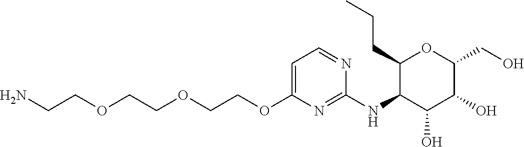 [SMILES: CCC[C@H]1O[C@H](CO)[C@H](O)[C@H](O)[C@H]1Nc1nccc(OCCOCCOCCN)n1] | Analogous to XB96 | 431.30 [M + H] |
| XB96 | 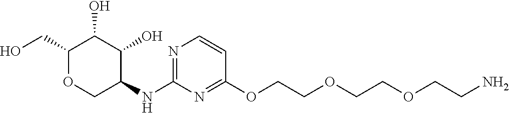 [SMILES: NCCOCCOCCOc1ccnc(N[C@H]2CO[C@H](CO)[C@H](O)[C@@H]2O)n1] | provided | 489.2 [M + H] |
| XB47 | 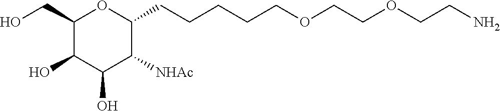 [SMILES: CC(=O)N[C@@H]1[C@@H](O)[C@@H](O)[C@@H](CO)O[C@@H]1CCCCCOCCOCCN] | Analogous to XB89 | 379.2 [M + H] |
| XB89 | 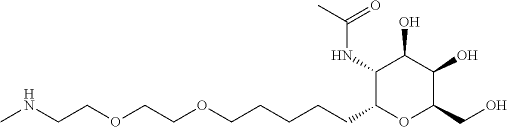 [SMILES: CNCCOCCOCCCCC[C@H]1O[C@H](CO)[C@H](O)[C@H](O)[C@H]1NC(C)=O] | provided | 393.15 [M + H] |
| XB87 | 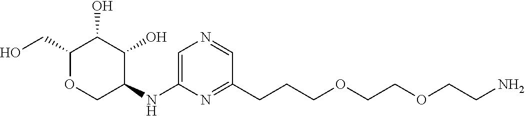 [SMILES: NCCOCCOCCCc1cncc(N[C@H]2CO[C@H](CO)[C@H](O)[C@@H]2O)n1] | Provided | 387.1 [M + H] |
| XB84 | 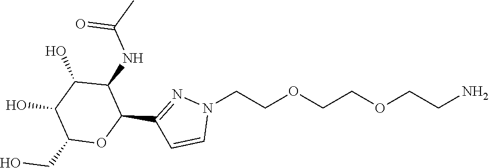 [SMILES: CC(=O)N[C@@H]1[C@@H](O)[C@@H](O)[C@@H](CO)O[C@@H]1c1ccn(CCOCCOCCN)n1] | Provided | 403.0 [M + H] |
| XB108 | 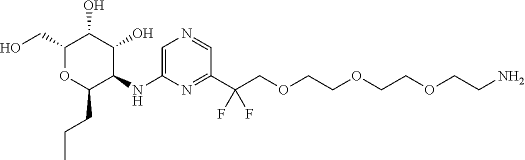 [SMILES: CCC[C@H]1O[C@H](CO)[C@H](O)[C@H](O)[C@H]1Nc1cncc(C(F)(F)COCCOCCOCCN)n1] | Analogous to XB96 (and XB80) | 495.25 [M + H] |
| XB109 | 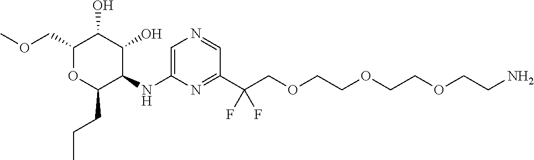 [SMILES: CCC[C@H]1O[C@H](COC)[C@H](O)[C@H](O)[C@H]1Nc1cncc(C(F)(F)COCCOCCOCCN)n1] | Analogous to XB83 (and XB80) | 509.30 [M + H] |
| XB95 | 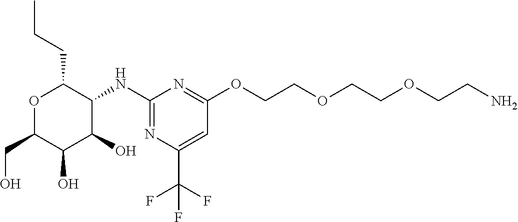 [SMILES: CCC[C@H]1O[C@H](CO)[C@H](O)[C@H](O)[C@H]1Nc1nc(OCCOCCOCCN)cc(C(F)(F)F)n1] | Provided | 499.4 [M + H] |
| XB52 | 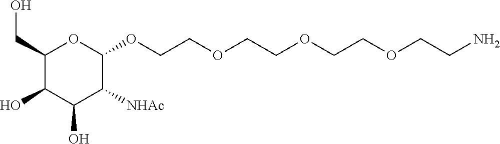 [SMILES: CC(=O)N[C@H]1[C@@H](OCCOCCOCCOCCN)O[C@H](CO)[C@H](O)[C@@H]1O] | provided | 397.15 [M + H] |
| XB54 | 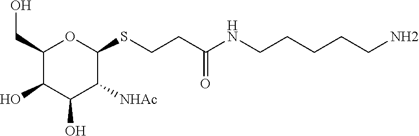 [SMILES: CC(=O)N[C@@H]1[C@@H](O)[C@@H](O)[C@@H](CO)O[C@H]1SCCC(=O)NCCCCCN] | Provided | 394.2 [M + H] |
| XB47 | 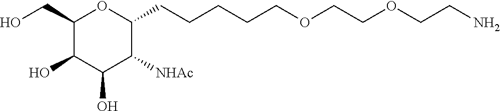 [SMILES: CC(=O)N[C@@H]1[C@@H](O)[C@@H](O)[C@@H](CO)O[C@@H]1CCCCCOCCOCCN] | Analogous to XB89 | 379.2 [M + H] |
| XB44A | 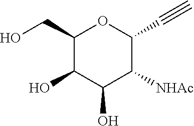 [SMILES: C#C[C@H]1O[C@H](CO)[C@H](O)[C@H](O)[C@H]1NC(C)=O] | Synthesis provided | 230.1 [M + H] |
| XB46 | 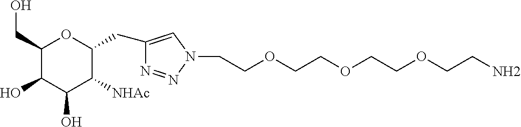 [SMILES: CC(=O)N[C@@H]1[C@@H](O)[C@@H](O)[C@@H](CO)O[C@@H]1Cc1cn(CCOCCOCCOCCN)nn1] | Provided | 1941.5 [M + H] |
| XB120 | 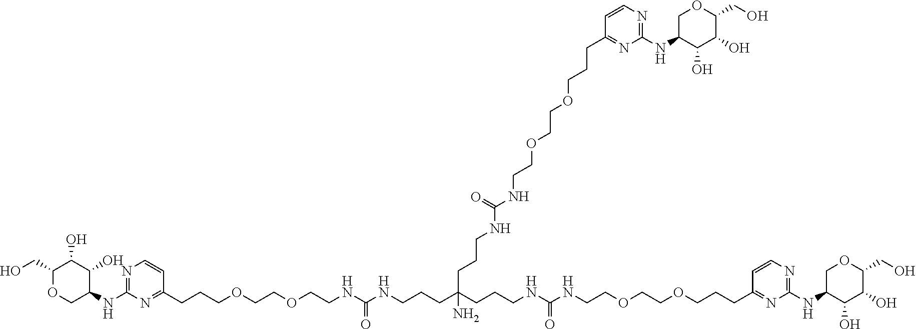 [SMILES: NC(CCCNC(=O)NCCOCCOCCCc1ccnc(N[C@H]2CO[C@H](CO)[C@H](O)[C@@H]2O)n1)(CCCNC(=O)NCCOCCOCCCc1ccnc(N[C@H]2CO[C@H](CO)[C@H](O)[C@@H]2O)n1)CCCNC(=O)NCCOCCOCCCc1ccnc(N[C@H]2CO[C@H](CO)[C@H](O)[C@@H]2O)n1] | Syntheis provided in Cmpd 2311 | 1439.4 [M + H] |
| XB121 | 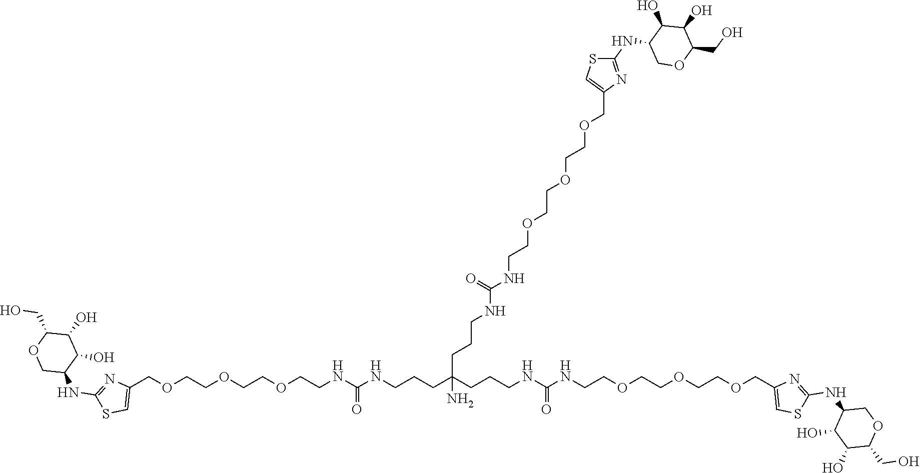 [SMILES: NC(CCCNC(=O)NCCOCCOCCOCc1csc(N[C@H]2CO[C@H](CO)[C@H](O)[C@@H]2O)n1)(CCCNC(=O)NCCOCCOCCOCc1csc(N[C@H]2CO[C@H](CO)[C@H](O)[C@@H]2O)n1)CCCNC(=O)NCCOCCOCCOCc1csc(N[C@H]2CO[C@H](CO)[C@H](O)[C@@H]2O)n1] | Synthesis provided in Cmpd 2310 | 1502.3 [M + H] |
| XB122 | 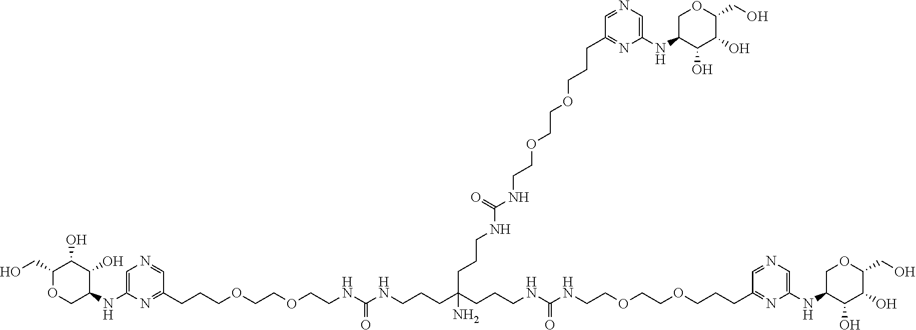 [SMILES: NC(CCCNC(=O)NCCOCCOCCCc1cncc(N[C@H]2CO[C@H](CO)[C@H](O)[C@@H]2O)n1)(CCCNC(=O)NCCOCCOCCCc1cncc(N[C@H]2CO[C@H](CO)[C@H](O)[C@@H]2O)n1)CCCNC(=O)NCCOCCOCCCc1cncc(N[C@H]2CO[C@H](CO)[C@H](O)[C@@H]2O)n1] | Synthesis provided in Cmpd 2303 | 1439.52 [M + H] |
| XB123 | 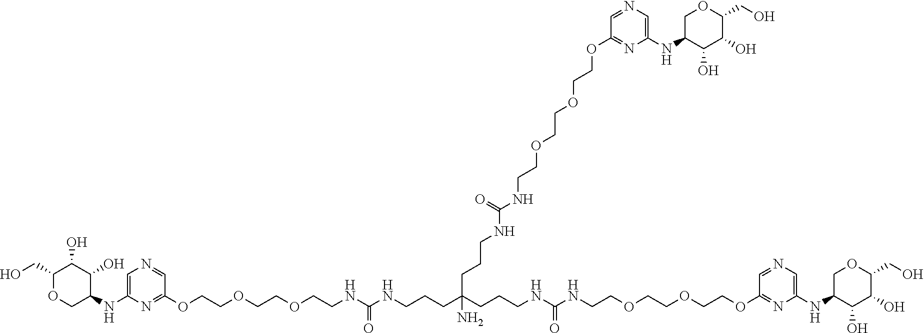 [SMILES: NC(CCCNC(=O)NCCOCCOCCOc1cncc(N[C@H]2CO[C@H](CO)[C@H](O)[C@@H]2O)n1)(CCCNC(=O)NCCOCCOCCOc1cncc(N[C@H]2CO[C@H](CO)[C@H](O)[C@@H]2O)n1)CCCNC(=O)NCCOCCOCCOc1cncc(N[C@H]2CO[C@H](CO)[C@H](O)[C@@H]2O)n1] | XB81 | 1445.9 [M + H] |
| XB124 | 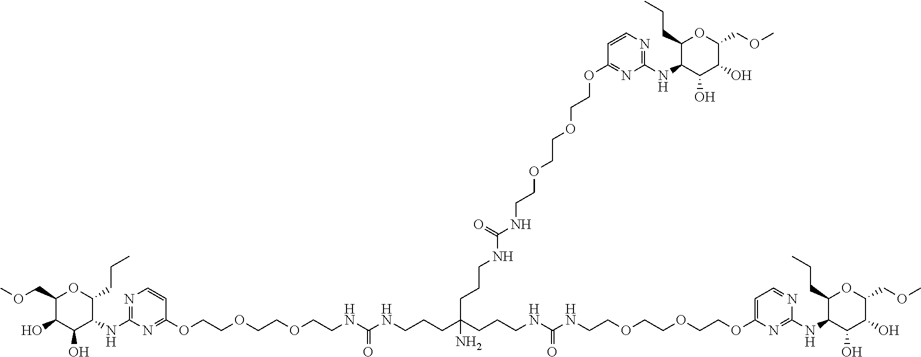 | Synthesis provided in Cmpd 2326 | 1613.5 [M + H] |
| XB130 | 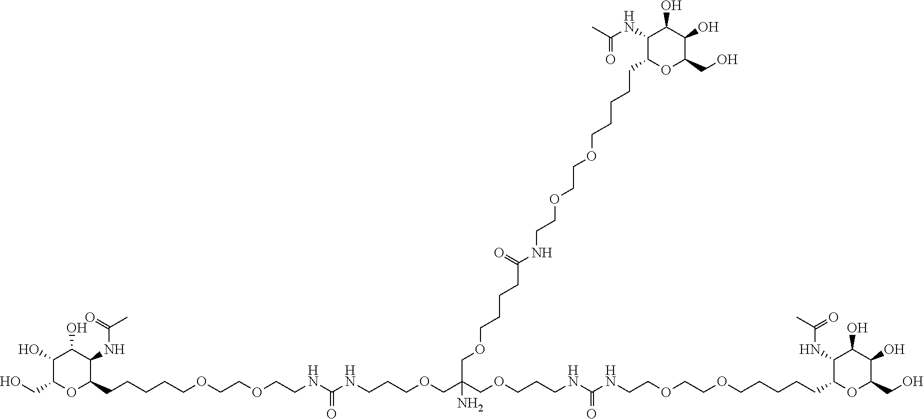 [SMILES: CC(=O)N[C@@H]1[C@@H](O)[C@@H](O)[C@@H](CO)O[C@@H]1CCCCCOCCOCCNC(=O)CCCCOCC(N)(COCCCNC(=O)NCCOCCOCCCCC[C@H]1O[C@H](CO)[C@H](O)[C@H](O)[C@H]1NC(C)=O)COCCCNC(=O)NCCOCCOCCCCC[C@H]1O[C@H](CO)[C@H](O)[C@H](O)[C@H]1NC(C)=O] | Synthesis provided in Cmpd 2317 | 1505.5 [M + H] |
| XB132 | 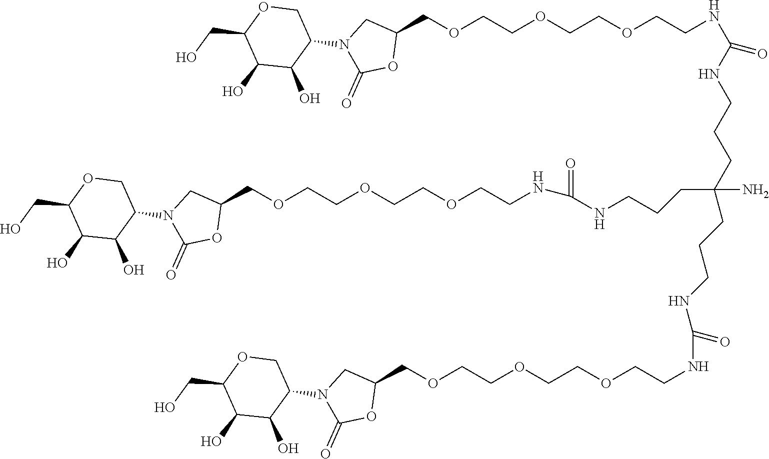 [SMILES: NC(CCCNC(=O)NCCOCCOCCOC[C@@H]1CN([C@H]2CO[C@H](CO)[C@H](O)[C@@H]2O)C(=O)O1)(CCCNC(=O)NCCOCCOCCOC[C@@H]1CN([C@H]2CO[C@H](CO)[C@H](O)[C@@H]2O)C(=O)O1)CCCNC(=O)NCCOCCOCCOC[C@@H]1CN([C@H]2CO[C@H](CO)[C@H](O)[C@@H]2O)C(=O)O1] | Synthesis provided in Cmpd 2306 | 1463.5 [M + H] |
| XB133 | 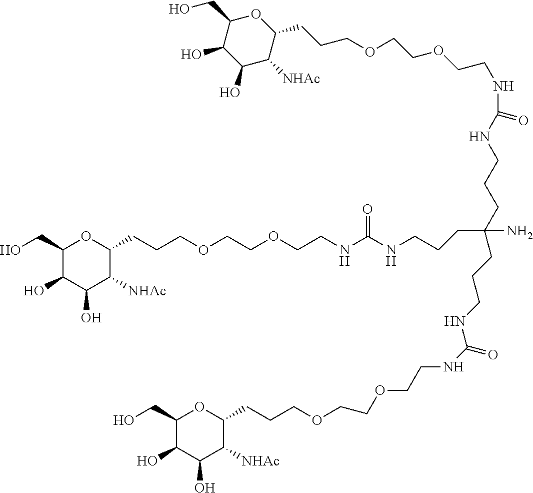 [SMILES: CC(=O)N[C@@H]1[C@@H](O)[C@@H](O)[C@@H](CO)O[C@@H]1CCCOCCOCCNC(=O)NCCCC(N)(CCCNC(=O)NCCOCCOCCC[C@H]1O[C@H](CO)[C@H](O)[C@H](O)[C@H]1NC(C)=O)CCCNC(=O)NCCOCCOCCC[C@H]1O[C@H](CO)[C@H](O)[C@H](O)[C@H]1NC(C)=O] | Synthesis provided in Cmpd 2313 | 1331.7 [M + H] |
| XB134 | 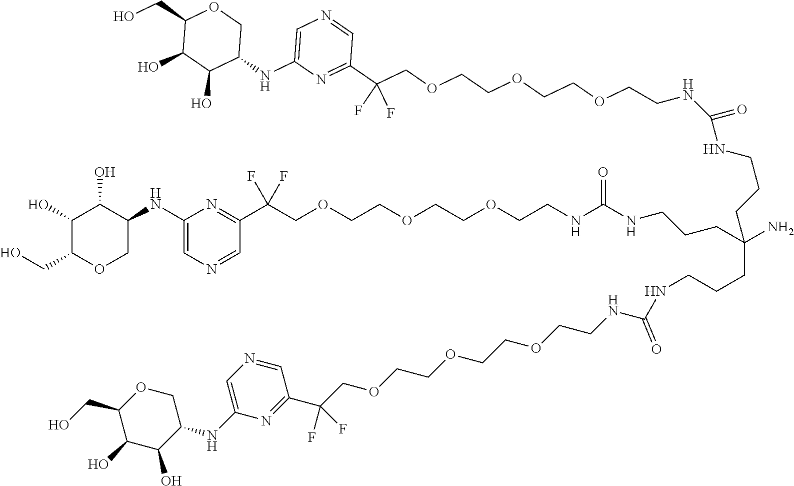 [SMILES: NC(CCCNC(=O)NCCOCCOCCOCC(F)(F)c1cncc(N[C@H]2CO[C@H](CO)[C@H](O)[C@@H]2O)n1)(CCCNC(=O)NCCOCCOCCOCC(F)(F)c1cncc(N[C@H]2CO[C@H](CO)[C@H](O)[C@@H]2O)n1)CCCNC(=O)NCCOCCOCCOCC(F)(F)c1cncc(N[C@H]2CO[C@H](CO)[C@H](O)[C@@H]2O)n1] | Synthesis provided in Cmpd 2327 | 1637.35 [M + H] |
| XB135 | 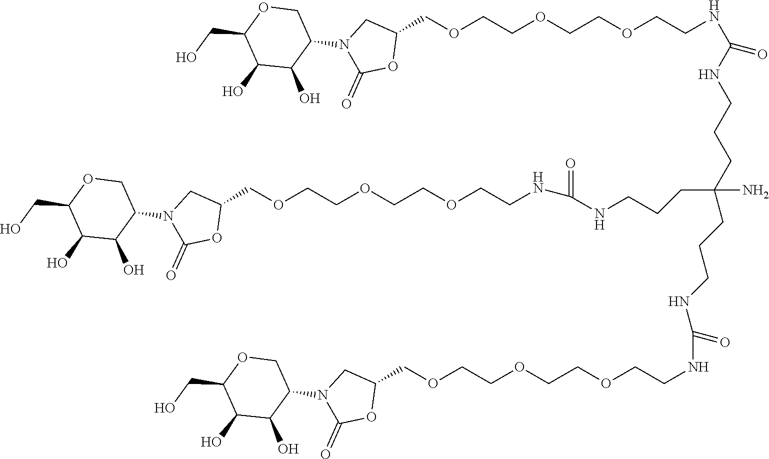 [SMILES: NC(CCCNC(=O)NCCOCCOCCOC[C@H]1CN([C@H]2CO[C@H](CO)[C@H](O)[C@@H]2O)C(=O)O1)(CCCNC(=O)NCCOCCOCCOC[C@H]1CN([C@H]2CO[C@H](CO)[C@H](O)[C@@H]2O)C(=O)O1)CCCNC(=O)NCCOCCOCCOC[C@H]1CN([C@H]2CO[C@H](CO)[C@H](O)[C@@H]2O)C(=O)O1] | Synthesis provided in Cmpd 2324 | 1463.4 [M + H] |
| XB136 | 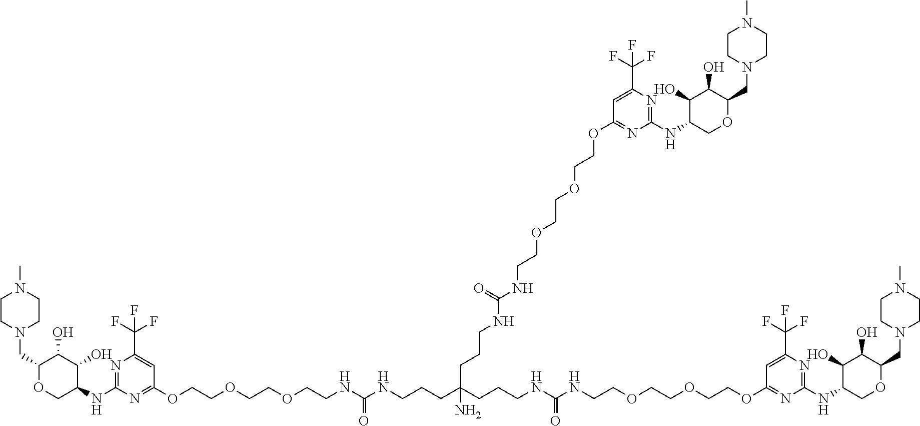 [SMILES: CN1CCN(C[C@H]2OC[C@H](Nc3nc(OCCOCCOCCNC(=O)NCCCC(N)(CCCNC(=O)NCCOCCOCCOc4cc(C(F)(F)F)nc(N[C@H]5CO[C@H](CN6CCN(C)CC6)[C@H](O)[C@@H]5O)n4)CCCNC(=O)NCCOCCOCCOc4cc(C(F)(F)F)nc(N[C@H]5CO[C@H](CN6CCN(C)CC6)[C@H](O)[C@@H]5O)n4)cc(C(F)(F)F)n3)[C@@H](O)[C@H]2O)CC1] | Synthesis provided in Cmpd 2312 | 949.1 [M +2H]++ |
| XB137 | 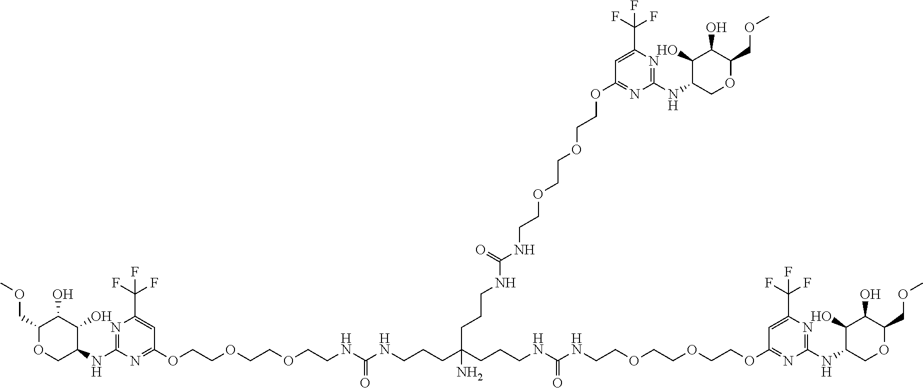 | Synthesis provided in Cmpd 2309 | 847.2 [M + 2H]++ |
| XB138 | 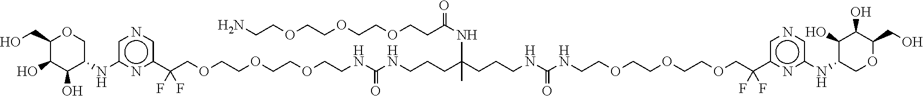 | Inter- mediate for Cmpd 2304 | 1319.5 [M + H] |
| XB105 | 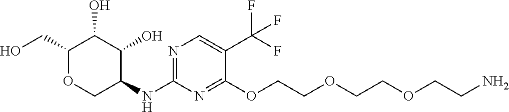 [SMILES: NCCOCCOCCOc1nc(N[C@H]2CO[C@H](CO)[C@H](O)[C@@H]2O)ncc1C(F)(F)F] | Analogous to XB92 | 457.3 [M + 1] |
| XB148 | 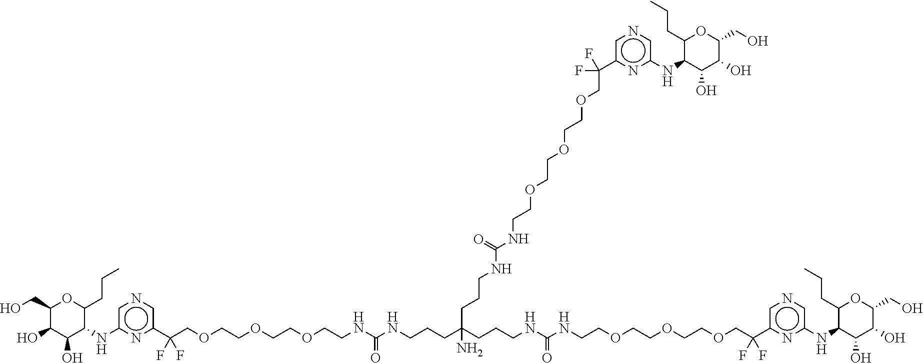 | Synthesis provided in Cmpd 2301 | 1765.0 [M + H] |
| XB149 | 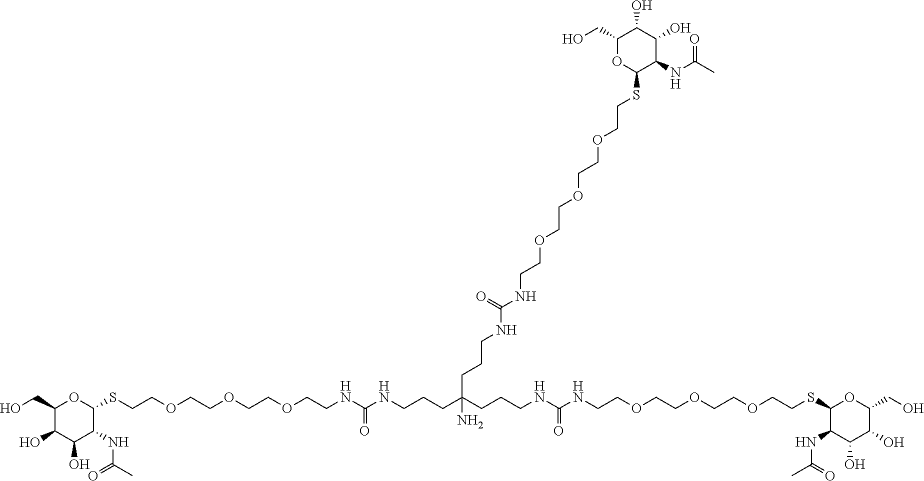 [SMILES: CC(=O)N[C@@H]1[C@@H](O)[C@@H](O)[C@@H](CO)O[C@@H]1SCCOCCOCCOCCNC(=O)NCCCC(N)(CCCNC(=O)NCCOCCOCCOCCS[C@H]1O[C@H](CO)[C@H](O)[C@H](O)[C@H]1NC(C)=O)CCCNC(=O)NCCOCCOCCOCCS[C@H]1O[C@H](CO)[C@H](O)[C@H](O)[C@H]1NC(C)=O] | Synthesis provided in Cmpd 2323 | 1517.6 [M + H] |
| XB150 | 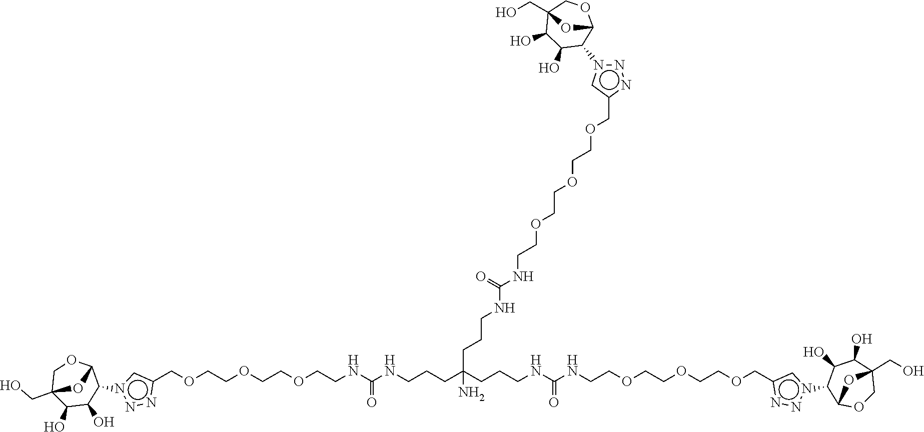 | Synthesis provided in Cmpd 2328 | 1493.4 [M + H] |
| XB151 | 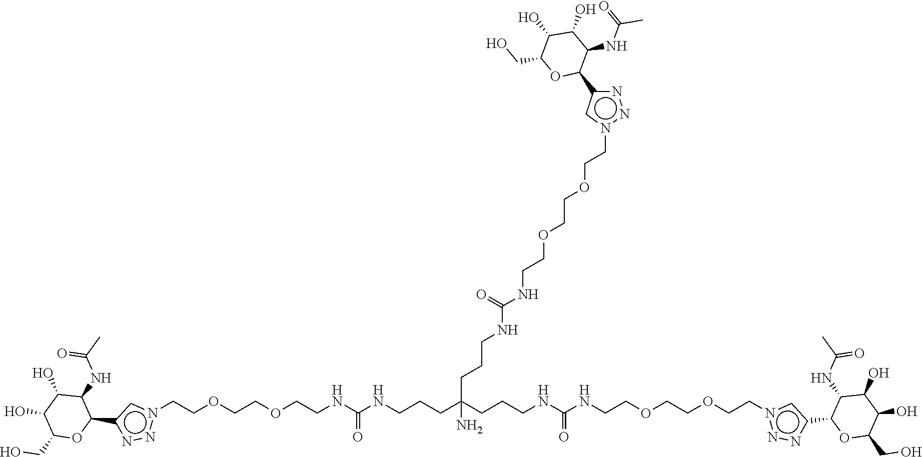 | Synthesis provided in Cmpd 2329 | 1491.4 |
| XB152 | 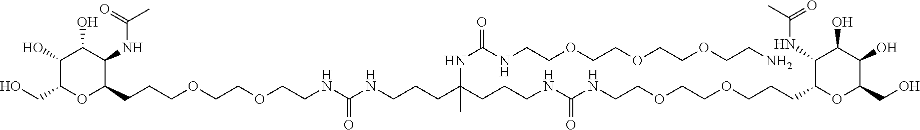 [SMILES: CC(=O)N[C@@H]1[C@@H](O)[C@@H](O)[C@@H](CO)O[C@@H]1CCCOCCOCCNC(=O)NCCCC(C)(CCCNC(=O)NCCOCCOCCC[C@H]1O[C@H](CO)[C@H](O)[C@H](O)[C@H]1NC(C)=O)NC(=O)NCCOCCOCCOCCN] | Synthesis provided in Cmpd 2318 | 1130.6 [M + H] |
| XB153 | 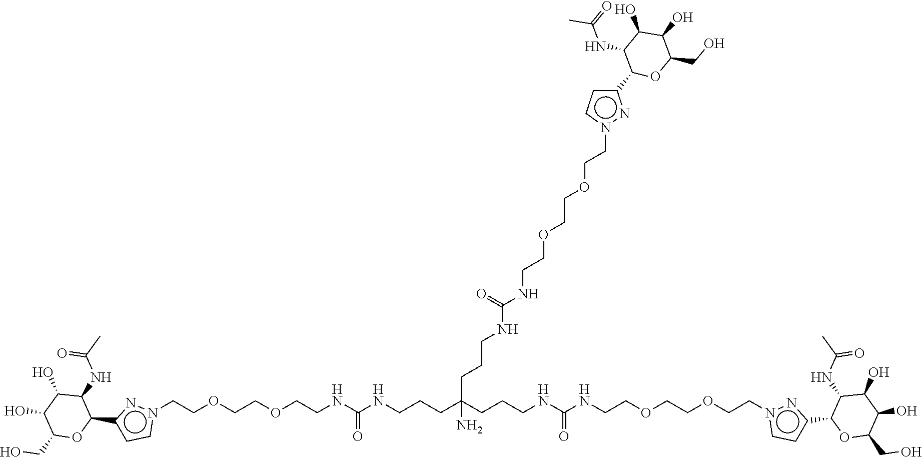 | Synthesis provided in Cmpd 2305 | 1487.5 [M + H] |

### Synthesis of ASGPR Targeting Compounds

### Synthesis of Compound 2307

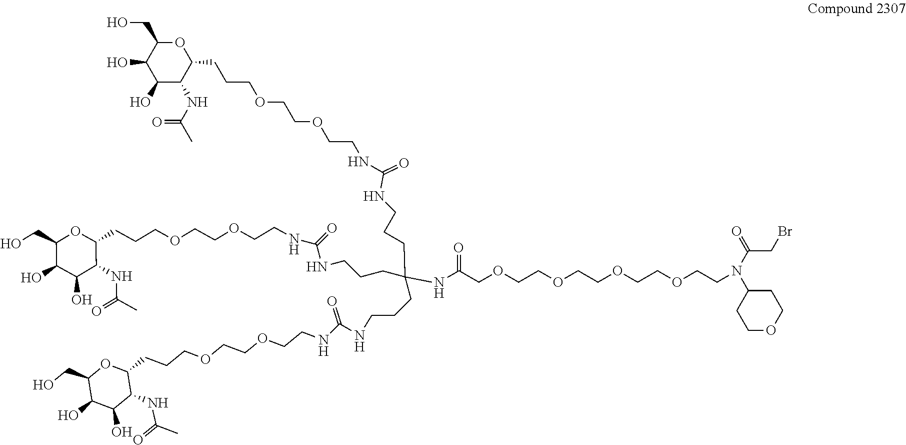 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1]

### Synthesis of N-(1,7-diamino-4-(3-aminopropyl)heptan-4-yl)-2,2,2-trifluoroacetamide (6a)

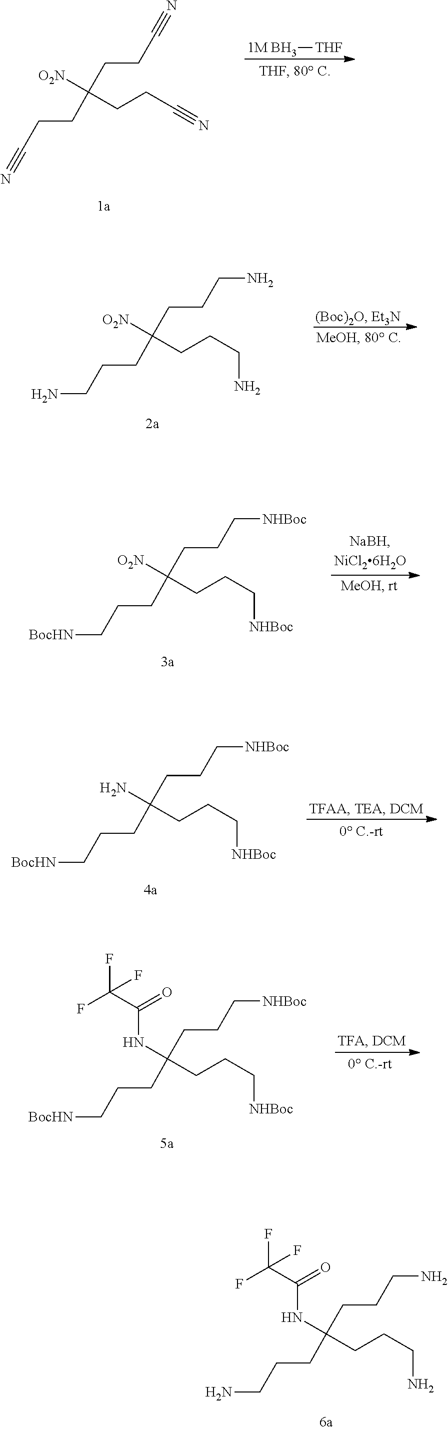

### Synthesis of 4-(3-aminopropyl)-4-nitroheptane-1,7-diamine (2a)

A solution of 4-(2-cyanoethyl)-4-nitroheptanedinitrile (1a, 5.0 g, 22.7 mmol) in dry tetrahydrofuran (25 mL) was cooled to 0° C. To this, borane-tetrahydrofuran complex (1M in THF, 114 mL, 5 eq., 114 mmol) was added slowly and the resulting reaction mixture was heated at 80° C. for 24 h. After completion, the reaction mixture was allowed to attain room temperature and conc. hydrochloric acid solution was added dropwise to quench excess borane complex. The resultant reaction mixture was heated to 50° C. for 30 min and then concentrated under reduced pressure. The residue was neutralized by adding 40% aqueous sodium hydroxide solution (80 mL) and extracted with dichloromethane. The organic layer was dried over anhydrous sodium sulphate, filtered and the filtrate was concentrated under reduced pressure to obtain 4-(3-aminopropyl)-4-nitroheptane-1,7-diamine (2a) as an off-white solid. Yield: 5.0 g, crude. LCMS: m/z 233.2 [M+H].

### Synthesis of di-tert-butyl (4-(3-((tert-butoxycarbonyl)amino)propyl)-4-nitroheptane-1,7-diyl)dicarbamate (3a)

To a solution of 4-(3-aminopropyl)-4-nitroheptane-1,7-diamine (2a, 8 g, 34.4 mmol) in methanol (150 mL), were added di-tert-butyl dicarbonate (26.1 mL, 3.3 eq., 114 mmol) and triethylamine (20.2 mL, 4.2 eq., 145 mmol) at 0° C. and the resultant reaction mixture was refluxed for 6 h. Progress of the reaction was monitored by TLC and LCMS. After completion, the reaction mixture was concentrated under reduced pressure to get crude which was purified by flash chromatography using silica gel (100-200 mesh; 20-40% ethyl acetate-heptane) to afford di-tert-butyl (4-(3-((tert-butoxycarbonyl)amino)propyl)-4-nitroheptane-1,7-diyl)dicarbamate (3a) as a colourless oil (which turns glassy solid when cooled at −20° C.). Yield: 9.0 g, 49.1%; LCMS: m/z 533.2 [M+H].

### Synthesis of di-tert-butyl (4-amino-4-(3-((tert-butoxycarbonyl)amino)propyl)heptane-1,7-diyl)dicarbamate (4a)

A suspension of nickel dichloride hexahydrate (7.14 g, 2 eq., 30 mmol) in methanol (80 mL) was cooled to 0° C. To this, sodium borohydride (1.55 g, 2.6 eq., 5 mmol) was added and the resulting black suspension was stirred for 30 min. Thereafter, the black suspension was further diluted with methanol (30 mL) followed by addition of di-tert-butyl (4-(3-((tert-butoxycarbonyl)amino)propyl)-4-nitroheptane-1,7-diyl)dicarbamate (3a, 8.0 g, 15 mmol) and sodium borohydride (1.55 g, 2.6 eq., 5 mmol). The resulting black suspension was stirred for 60 min. Subsequently, a last portion of sodium borohydride (1.55 g, 2.6 eq., 5 mmol) was added and the reaction mixture was stirred at room temperature for 4 h. After completion, the black suspension was filtrated through a celite bed, and the filtrate was concentrated under reduced pressure. The residue was diluted by adding water and extracted with dichloromethane. The organic layer was dried over anhydrous sodium sulphate, filtrated, filtered and the filtrate was concentrated under reduced pressure to afford di-tert-butyl (4-amino-4-(3-((tert-butoxycarbonyl)amino)propyl)heptane-1,7-diyl)dicarbamate (4a) as a white solid. Yield: 6.0 g, 79.5%; LCMS: m/z 503.2 [M+H]. ¹H NMR (400 MHz, DMSO-d₆ with D₂O): δ 2.83 (t, J=6.80 Hz, 6H), 1.34 (s, 27H), 1.32-1.28 (m, 6H), 1.14-1.12 (m, 6H).

### Synthesis of di-tert-butyl (4-(3-((tert-butoxycarbonyl)amino)propyl)-4-(2,2,2-trifluoroacetamido)heptane-1,7-diyl)dicarbamate (5a)

A solution of di-tert-butyl (4-amino-4-(3-((tert-butoxycarbonyl)amino)propyl)heptane-1,7-diyl)dicarbamate (4a, 1.0 g, 1.99 mmol) and triethylamine (1.39 mL, 5.0 eq., 9.95 mmol) in dry dichloromethane (10 mL) was cooled to 0° C. To this, trifluoroacetic anhydride (1.25 mL, 3.0 eq., 5.97 mmol) was added slowly, and the resulting reaction mixture was stirred at room temperature for 12 h. Progress of the reaction was monitored by TLC and LCMS. After completion, the reaction mixture was concentrated under reduced pressure to obtain crude. The crude was purified by flash chromatography using silica gel (100-200 mesh, eluent: 10-20% ethyl acetate-heptane) to afford di-tert-butyl (4-(3-((tert-butoxycarbonyl)amino)propyl)-4-(2,2,2-trifluoroacetamido)heptane-1,7-diyl)dicarbamate (5a) as a colorless, viscous liquid. Yield: 0.75 g, 63.0%; LCMS: m/z 599.45 [M+H].

### Synthesis of N-(1,7-diamino-4-(3-aminopropyl)heptan-4-yl)-2,2,2-trifluoroacetamide (6a)

To a stirred solution of di-tert-butyl (4-(3-((tert-butoxycarbonyl)amino)propyl)-4-(2,2,2-trifluoroacetamido)heptane-1,7-diyl)dicarbamate (5a, 0.75 g, 1.25 mmol) in dichloromethane (8 mL), was added trifluoroacetic acid (3 mL) at 0° C. The resultant reaction mixture was stirred at room temperature for 3 h. Progress of the reaction was monitored by TLC and LCMS. After completion, the reaction mixture was concentrated under reduced pressure to obtain crude. The was purified by reverse phase prep-HPLC (30-40% acetonitrile in water with 0.1% ammonium hydroxide). Fractions containing the desired product were combined and lyophilized to dryness to afford N-(1,7-diamino-4-(3-aminopropyl)heptan-4-yl)-2,2,2-trifluoroacetamide (6a) as an off-white solid. Yield: 0.070 g, 18.7%; LCMS m/z 299.25 [M+H]. ¹H-NMR (400 MHz, DMSO-d₆ with D₂O): δ 3.13 (t, J=6.72 Hz, 1H), 2.85 (bs, 2H), 1.70-1.53 (m, 6H), 1.49-1.40 (m, 1H), 1.27-1.21 (in, 1OH).

### Synthesis of 14-((4-nitro-N-(tetrahydro-2H-pyran-4-yl)phenyl)sulfonamido)-3,6,9,12-tetraoxatetradecanoic acid (4b)

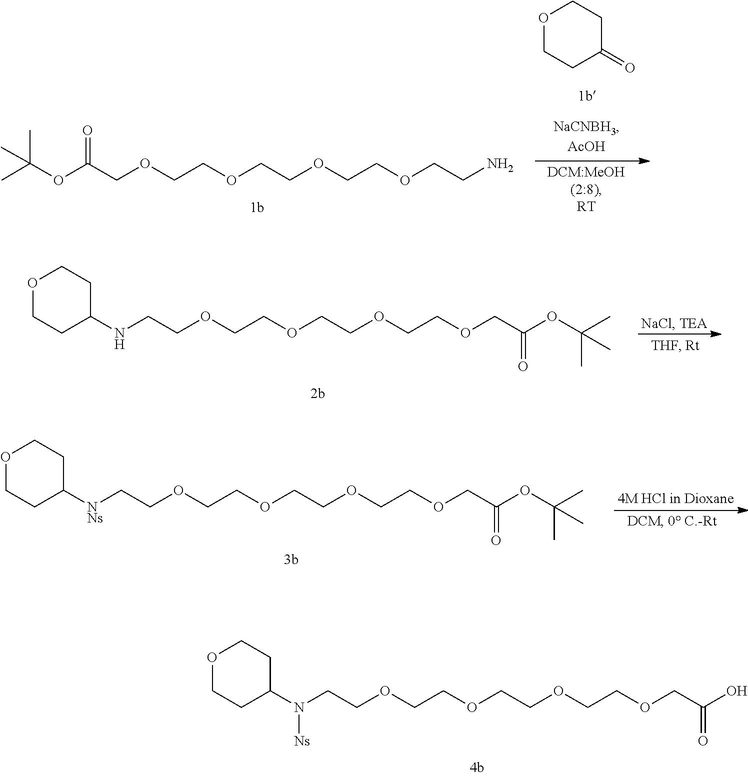

### Synthesis of tert-butyl 14-((tetrahydro-2H-pyran-4-yl)amino)-3,6,9,12-tetraoxatetradecanoate (2b)

To a stirred solution of tert-butyl 14-amino-3,6,9,12-tetraoxatetradecanoate (1b, 2 g, 6.51 mmol) in methanol (16 mL) and dichloromethane (4 mL) were added tetrahydro-4H-pyran-4-one (1b′, 0.717 g, 1.1 eq., 7.16 mmol), acetic acid (0.0372 mL, 0.1 eq., 0.651 mmol) and stirred for 1 h at room temperature. Then sodium cyanoborohydride (0.467 g, 1.2 eq., 7.81 mmol) was added and stirred for another 4 h. After completion, that reaction mixture was concentrated and portioned between water and ethyl acetate. The ethyl acetate part was dried over anhydrous sodium sulphate, filtered, and concentrated to give crude which was purified by reverse phase prep HPLC (70-75% acetonitrile in water with 0.1% TFA). Fractions containing desired product were combined and lyophilized to dryness to afford tert-butyl 14-((tetrahydro-2H-pyran-4-yl)amino)-3,6,9,12-tetraoxatetradecanoate (2b) as sticky solid. Yield: 0.8 g, 32%; LCMS m/z 392.85 [M+H].

### Synthesis of tert-butyl 14-((4-nitro-N-(tetrahydro-2H-pyran-4-yl)phenyl)sulfonamido)-3,6,9,12-tetraoxatetradecanoate (3b)

To a stirred solution of tert-butyl 14-((tetrahydro-2H-pyran-4-yl)amino)-3,6,9,12-tetraoxatetradecanoate (2b, 0.85 g, 1 eq., 2.17 mmol) in tetrahydrofuran (9.0 mL) was added triethylamine (3.05 mL, 10 eq., 21.7 mmol) and 4-nitrobenzenesulfonyl chloride (2.41 g, 5.0 eq., 10.9 mmol) at 0° C. and stirred the reaction mixture at 30° C. for 3 h. Progress of reaction was monitored by TLC. After completion, reaction mixture was diluted with water and extracted with dichloromethane to afford crude which was purified by silica gel column chromatography (60% ethyl acetate/hexane) to afford tert-butyl 14-((4-nitro-N-(tetrahydro-2H-pyran-4-yl)phenyl)sulfonamido)-3,6,9,12-tetraoxatetradecanoate (3b) as yellow sticky liquid. Yield: 0.66 g, 52.72%; LCMS m/z 577.05 [M+H].

### Synthesis of 14-((4-nitro-N-(tetrahydro-2H-pyran-4-yl)phenyl)sulfonamido)-3,6,9,12-tetraoxatetradecanoic acid (4b)

To a solution of tert-butyl 14-((4-nitro-N-(tetrahydro-2H-pyran-4-yl)phenyl)sulfonamido)-3,6,9,12-tetraoxatetradecanoate (3b, 0.660 g, 1.14 mmol) in dichloromethane (3 mL) was added hydrogen chloride solution (3 ml, 4M in 1,4-dioxane) dropwise at 0° C. and reaction was stirred at same temperature for 3 h. After completion, reaction mixture was concentrated and co-distilled with dichloromethane to afford crude which was purified by reverse phase prep HPLC (80% acetonitrile in water with 0.1% TFA). Fractions containing desired product were combined and lyophilized to dryness to afford 14-((4-nitro-N-(tetrahydro-2H-pyran-4-yl)phenyl)sulfonamido)-3,6,9,12-tetraoxatetradecanoic acid (4b) as colorless viscous liquid. Yield: 0.475 g, 80%. LCMS m/z 519.4 [M-1]. ¹H NMR (400 MHz, DMSO-d₆) δ 12.57 (s, 1H), 8.36 (d, J=8.8 Hz, 2H), 8.14 (d, J=8.8 Hz, 2H), 3.99 (s, 2H), 3.88-3.84 (m, 1H), 3.79 (dd, J=4.0, 11.2 Hz, 2H), 3.57-3.48 (m, 15H), 3.29-3.26 (m, 3H), 1.74-1.64 (m, 2H), 1.36-1.34 (m, 2H).

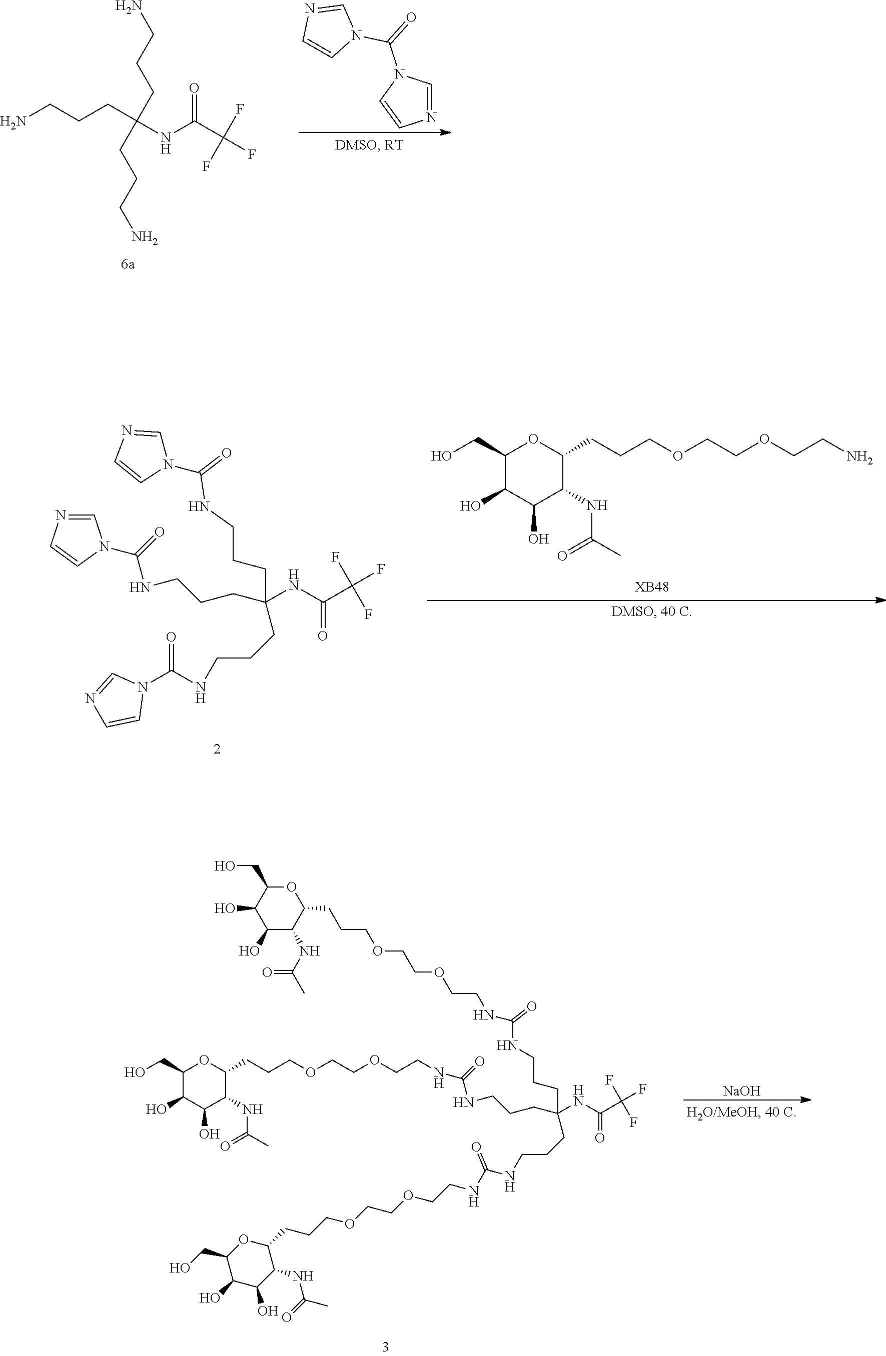 [SMILES: NCCCC(CCCN)(CCCN)NC(=O)C(F)(F)F ; O=C(n1ccnc1)n1ccnc1 ; O=C(NCCCC(CCCNC(=O)n1ccnc1)(CCCNC(=O)n1ccnc1)NC(=O)C(F)(F)F)n1ccnc1 ; CC(=O)N[C@@H]1[C@@H](O)[C@@H](O)[C@@H](CO)O[C@@H]1CCCOCCOCCN ; CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)C(F)(F)F ; "NaOH"]

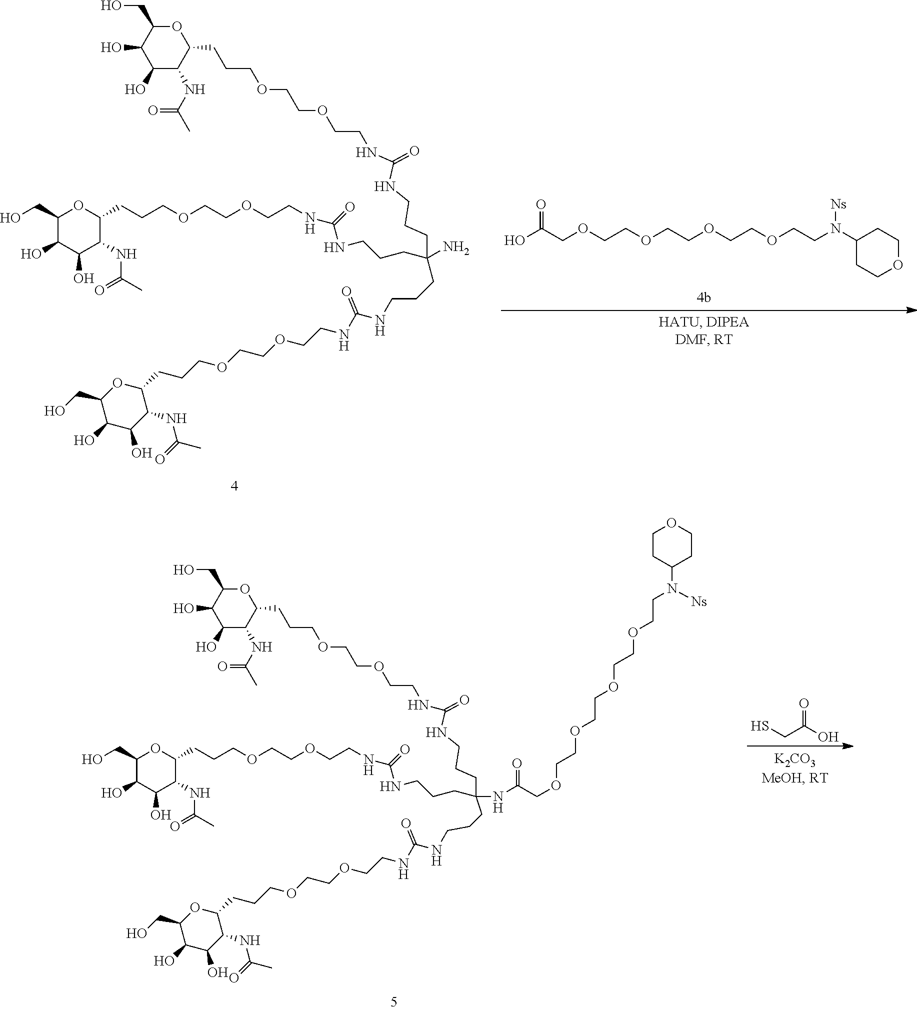

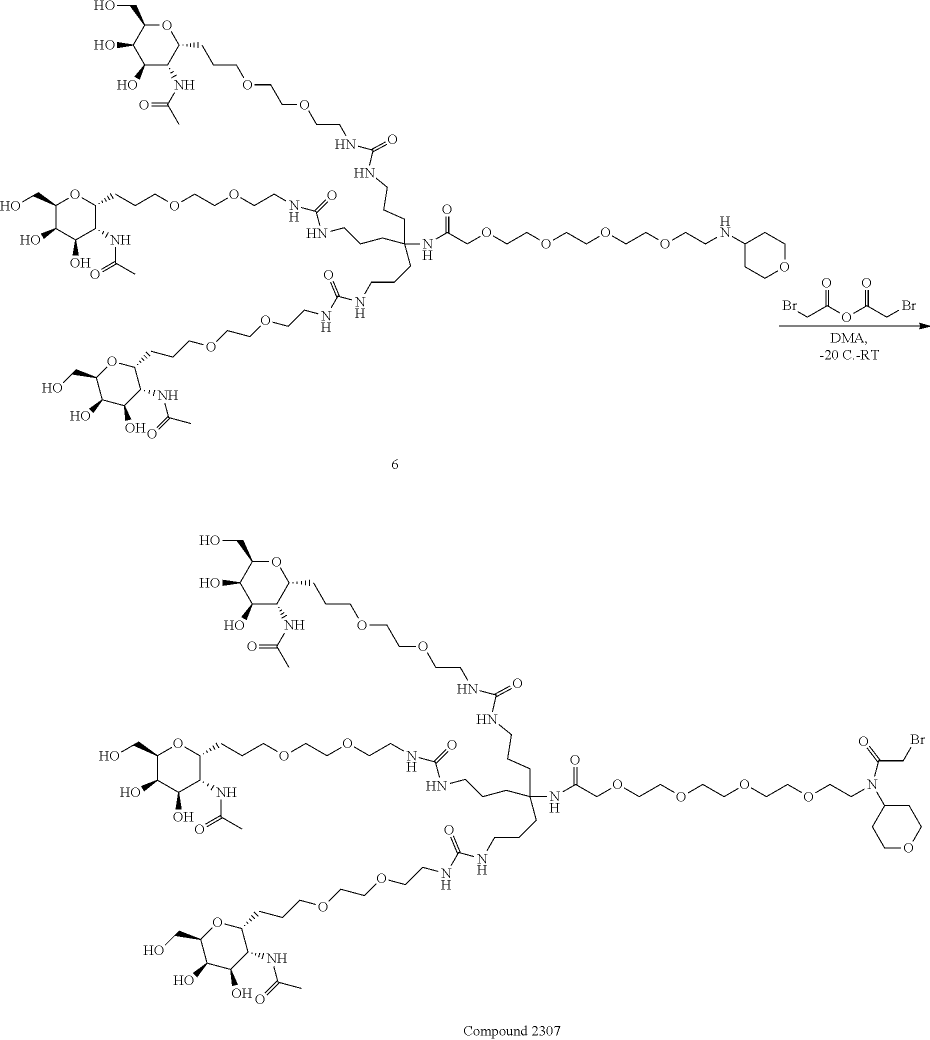 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)COCCOCCOCCOCCNC1CCOCC1 ; O=C(CBr)OC(=O)CBr ; CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1]

### Synthesis of N,N′-(4-(3-(1H-imidazole-1-carboxamido)propyl)-4-(2,2,2-trifluoroacetamido)heptane-1,7-diyl)bis(1H-imidazole-1-carboxamide) (2)

A solution of N-[4-amino-1,1-bis(3-aminopropyl)butyl]-2,2,2-trifluoro-acetamide; 2,2,2-trifluoroacetic acid (6a) (57.0 mg, 0.0890 mmol, 1.0 eq.) in 0.6 mL DMSO was added carbonyldiimidazole (108.2 mg, 0.668 mmol, 7.5 eq). The reaction solution stirred under nitrogen atmosphere and ambient temperature for 1 hr. The reaction mixture was diluted with 1 mL of DMSO and purified by reverse phase HPLC (5-100% acetonitrile in water). Fractions containing desired product were combined and lyophilized to dryness to afford N-[7-(imidazole-1-carbonylamino)-4-[3-(imidazole-1-carbonylamino)propyl]-4-[(2,2,2-trifluoroacetyl)amino]heptyl]imidazole-1-carboxamide (2) as a white solid. Yield: 55 mg (106%); LCMS m/z 581.04 [M+H].

### Synthesis of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2,2,2-trifluoro-acetamide (3)

To a stirring solution of N-[(2R,3R,4R,5R,6R)-2-[3-[2-(2-aminoethoxy)ethoxy]propyl]-4,5-dihydroxy-6-(hydroxymethyl)tetrahydropyran-3-yl]acetamide (XB48) (67.3 mg, 0.192 mmol, 3.4 eq.) in 0.6 mL of DMSO was added a solution of N-[7-(imidazole-1-carbonylamino)-4-[3-(imidazole-1-carbonylamino)propyl]-4-[(2,2,2-trifluoroacetyl)amino]heptyl]imidazole-1-carboxamide (2) (32.7 mg, 0.0564 mmol, 1.0 eq) in 0.3 mL of DMSO. The reaction solution stirred under nitrogen atmosphere at 40 C for 3 hrs. The reaction mixture was diluted with 1 mL of DMSO and purified by reverse phase HPLC (5-40% acetonitrile in water with 0.1% TFA modifier). Fractions containing desired product were combined and lyophilized to dryness to afford N-[4-[2-[2-[33- [(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2 -yl]propoxy]ethoxy]ethylcarbamoylamino]propyl]butyl]-2,2,2-trifluoro-acetamide (3) as a white solid. Yield: 65.3 mg (81%); LCMS m/z 1427.4 [M+H].

### Synthesis of N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (4)

To a solution of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2,2,2-trifluoro-acetamide (3) (53.9 mg, 0.0378 mmol, 1.0 eq.) in 0.9 mL of methanol was added 0.4 mL of aqueous 5N sodium hydroxide. The reaction stirred at 40 C overnight, then the solution was concentrated to -¼ original volume under a stream of nitrogen. The concentrated crude mixture was diluted with 2 mL of H₂O and purified by reverse phase HPLC (2-40% acetonitrile in water with 20 mM ammonium hydroxide modifier). Fractions containing desired product were combined and lyophilized to dryness to afford N-[(2R,3R,4R,5R,6R)-2-[3-[2-[2-[[7-[[22-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (4) as a white solid. Yield: 40.3 mg (75%); LCMS m/z 1331.6 [M+H].

### Synthesis of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-[(2-nitrophenyl)sulfonyl-tetrahydropyran-4-yl-amino]ethoxy]ethoxy]ethoxy]ethoxy]acetamide (5)

A solution of 2-[2-[2-[2-[2-[(4-nitrophenyl)sulfonyl-tetrahydropyran-4-yl-amino]ethoxy]ethoxy]ethoxy]ethoxy]acetic acid (4b) (19.7 mg, 0.0378 mmol, 1.2 eq.) in 0.2 mL of DMF was added [dimethylamino(triazolo[4,5-b]pyridin-3-yloxy)methylene]-dimethyl-ammonium; hexafluorophosphate (HATU) (15.1 mg, 0.0397 mmol, 1.3 eq.) and diisopropylethylamine (DIPEA) (16.1 mL, 0.0924 mmol, 3.0 eq.). The solution stirred under nitrogen atmosphere at ambient temperature for −5 min, then was added to a stirring solution of N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[1(2R,3R,4R,5R,6R)-3-acetamido-4,5-dihydroxy-6-(hydroxymethyl)tetrahydropyran-2 -yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (4) (40.3 mg, 0.0303 mmol, 1.0 eq) dissolved in 0.7 mL of DMF. The reaction solution stirred under nitrogen atmosphere at ambient temperature for 1 hr. The reaction mixture was diluted with 1 mL of DMSO and purified by reverse phase HPLC (5-100% acetonitrile in water with 0.1% TFA modifier). Fractions containing desired product were combined and lyophilized to dryness to afford N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-[(2-nitrophenyl)sulfonyl-tetrahydropyran-4-yl-amino]ethoxy]ethoxy]ethoxy]ethoxy]acetamide (5) as a white solid. Yield: 38.3 mg (69%); LCMS m/z 1834.5 [M+H].

### Synthesis of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-(tetrahydropyran-4-ylamino)ethoxy]ethoxy]ethoxy]ethoxy]acetamide (6)

To a solution of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-[(2-nitrophenyl)sulfonyl-tetrahydropyran-4-yl-amino]ethoxy]ethoxy]ethoxy]ethoxy]acetamide (5) (36.6 mg, 0.0200 mmol, 1.0 eq.) in 1.5 mL of methanol was added K₂CO₃ (42.1 mg, 0.305 mmol, 15.3 eq.) and 2-thioglycolic acid (7 mL, 0.100 mmol, 5.0 eq.). The reaction solution stirred under nitrogen atmosphere at ambient temperature for 4 hrs. The reaction solution was concentrated under a stream of nitrogen to yellow solid. The concentrated crude solid was diluted with 2 mL of H₂O and purified by reverse phase HPLC (2-40% acetonitrile in water with 20 mM ammonium hydroxide modifier). Fractions containing desired product were combined and lyophilized to dryness to afford N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-(tetrahydropyran-4-ylamino)ethoxy]ethoxy]ethoxy]ethoxy]acetamide (6) as a white solid. Yield: 26.9 mg (82%); LCMS m/z 1649.6 [M+H].

### Synthesis of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2 -yl]propoxy]ethoxy]ethylcarbamoylamino]propyl]butyl]-2-[2-[2-[2-[2-[(2-bromoacetyl)-tetrahydropyran-4-yl-amino]ethoxy]ethoxy]ethoxy]ethoxy]acetamide (Compound 2307)

A solution of N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-(tetrahydropyran-4-ylamino)ethoxy]ethoxy]ethoxy]ethoxy]acetamide (6) (11.3 mg, 0.0068 mmol, 1.0 eq.) in 0.3 mL of dimethylacetamide (DMA) was stirred under nitrogen atmosphere at ~−20 C to form a white slurry, then a solution of 2-bromoacetic anhydride (2.4 mg, 0.0092 mmol, 1.3 eq.), dissolved in 0.2 mL of DMA, was added dropwise over 2 min. The reaction slurry continued to stir cold while warming to ambient temperature to form a clear solution. After 40 min., the reaction mixture was diluted with 1 mL of DMA and purified by reverse phase HPLC (2-40% acetonitrile in water with 0.10% TFA modifier). Fractions containing desired product were combined and lyophilized to dryness to afford N-[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1,1-bis[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]butyl]-2-[2-[2-[2-[2-[(2-bromoacetyl)-tetrahydropyran-4-yl-amino]ethoxy]ethoxy]ethoxy]ethoxy]acetamide (Compound 2307) as a white solid. Yield: 5.6 mg (46%); LCMS m/z 1770.4 [M+H].

### Method D: Synthesis of Compound 2313 from XB48

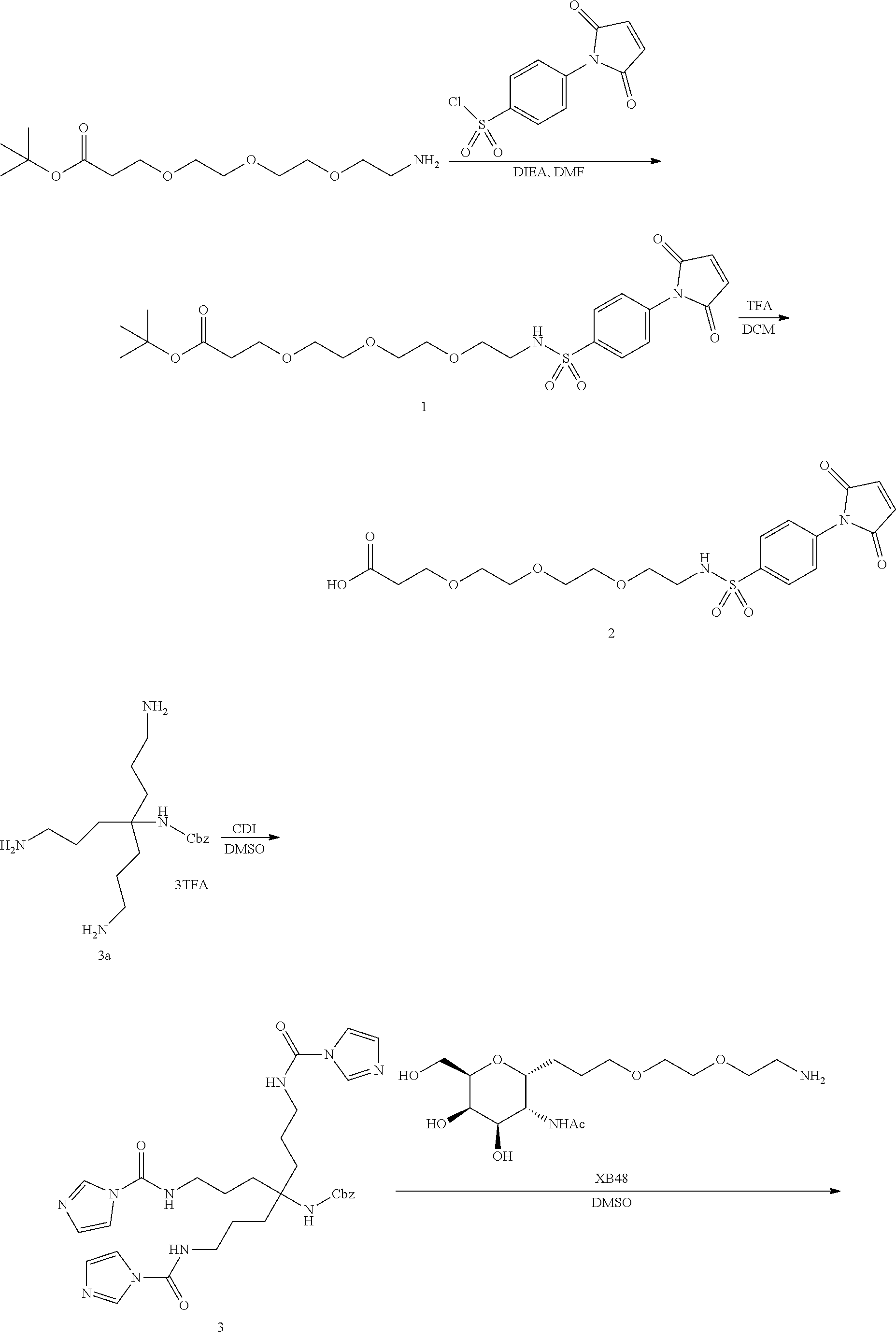

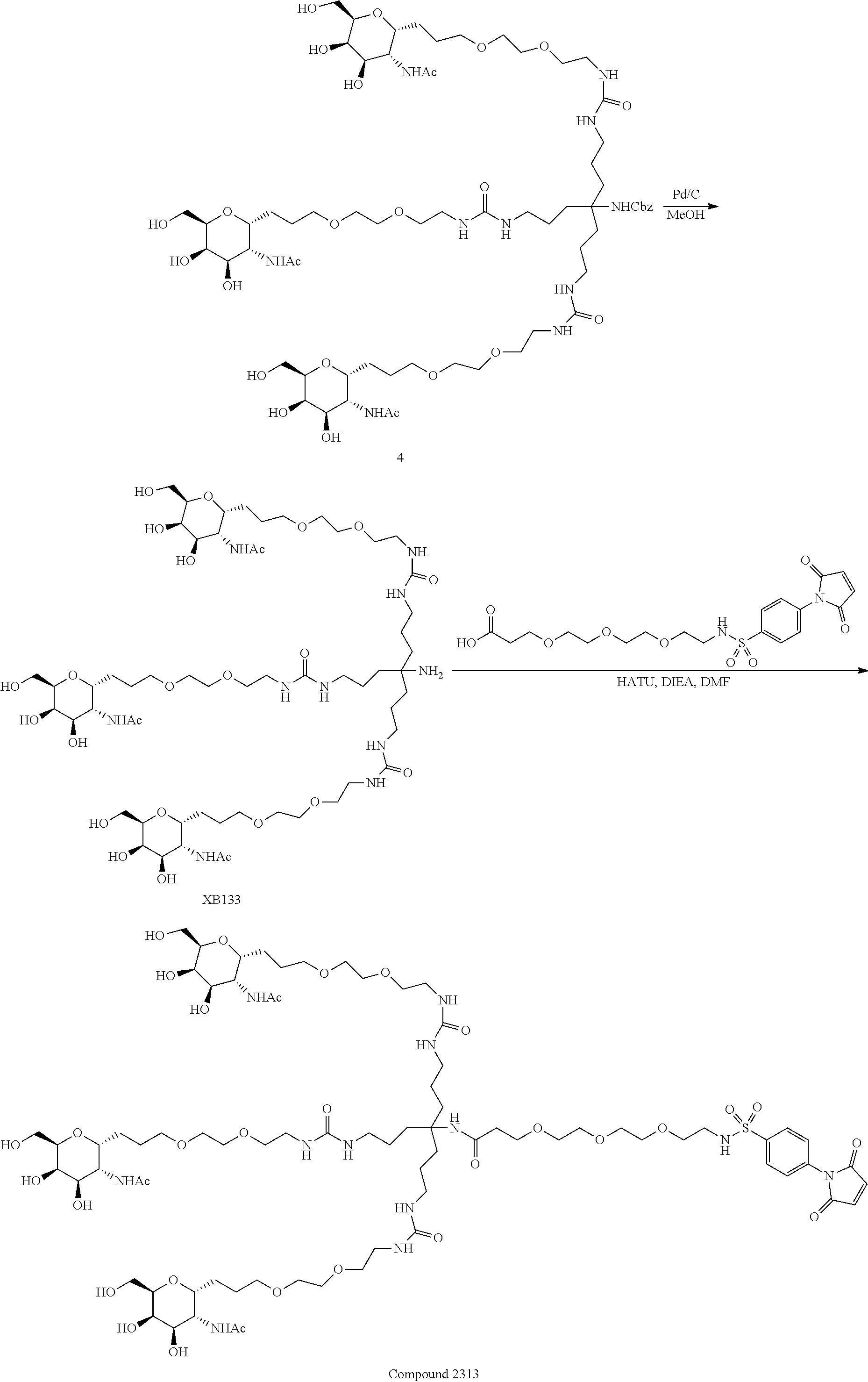

### Synthesis of tert-butyl 3-(2-(2-(2-((4-(2,5-dioxo-2,5-dihydro-1H-pyrrol-1-yl)phenyl)sulfonamido)ethoxy)ethoxy)ethoxy)propanoate (1)

To a mixture of 4-(2,5-dioxopyrrol-1-yl)benzenesulfonyl chloride (1.02 eq, 160 mg, 0.59 mmol) in DMF (1.6 mL) at 0° C. was added DIPEA (1.30 eq, 0.13 mL, 0.75 mmol) and a solution of tert-butyl 3-(2-(2-(2-aminoethoxy)ethoxy)ethoxy)propanoate (1.00 eq, 161 mg, 0.58 mmol) in DMF (1.7 mL). The mixture was stirred at 0° C. for 1 h then purified by prep HPLC (10-80% o MeCN/water with 0.100 TFA) to give 1 as a clear syrup. Yield: 174.4 mg, 62% o. LCMS m/z 457.0 [M-Boc+H].

### Synthesis of 3-(2-(2-(2-((4-(2,5-dioxo-2,5-dihydro-1H-pyrrol-1-yl)phenyl)sulfonamido)ethoxy)ethoxy)ethoxy)propanoic acid (2)

To a mixture of tert-butyl 3-(2-(2-(2-((4-(2,5-dioxo-2,5-dihydro-1H-pyrrol-1-yl)phenyl)sulfonamido)ethoxy)ethoxy)ethoxy)propanoate (1, 1.00 eq, 90.0 mg, 0.18 mmol) in DCM (1 mL) was added TFA (1 mL). The mixture was stirred at rt for 30 minutes then concentrated and purified by prep HPLC (5-70% MeCN/water) to give 2 as clear syrup. Yield: 66 mg, 82%. LCMS m/z 457.1 [M+H].

### Synthesis of benzyl (4-(3-(1H-imidazole-1-carboxamido)propyl)-1,7-bis(1H-imidazole-1-carboxamido)heptan-4-yl)carbamate (3)

To a mixture of carbonyldiimidazole (7.00 eq, 162 mg, 1.00 mmol) in DMSO (0.5 mL) was added a solution of benzyl N-[4-amino-1,1-bis(3-aminopropyl)butyl]carbamate trifluoroacetate salt (3a, 1.00 eq, 97.0 mg, 0.14 mmol) in DMSO (1.3 mL) dropwise. The mixture was stirred at rt for 1 h then diluted with EtOAc, washed with water (lx) and the aqueous layer was extracted with EtOAc. The combined organic layer was washed with water (lx) and brine (lx), dried, concentrated, and purified by column (0-10% MeOH/DCM) to give 3 as clear syrup. Yield: 62.7 mg, 71%. LCMS m/z 619.1 [M+H].

### Synthesis of benzyl (1,31-bis((2R,3R,4R,5R,6R)-3-acetamido-4,5-dihydroxy-6-(hydroxymethyl)tetrahydro-2H-pyran-2-yl)-16-(15-((2R,3R,4R,5R,6R)-3-acetamido-4,5-dihydroxy-6-(hydroxymethyl)tetrahydro-2H-pyran-2-yl)-5-oxo-9,12-dioxa-4,6-diazapentadecyl)-11,21-dioxo-4,7,25,28-tetraoxa-10,12,20,22-tetraazahentriacontan-16-yl)carbamate (4)

To a mixture of benzyl (4-(3-(1H-imidazole-1-carboxamido)propyl)-1,7-bis(1H-imidazole-1-carboxamido)heptan-4-yl)carbamate (3, 1.00 eq, 93.4 mg, 0.15 mmol) in DMSO (0.8 mL) was added a solution of N-[(2R,3R,4R,5R,6R)-2-[3-[2-(2-aminoethoxy)ethoxy]propyl]-4,5-dihydroxy-6-(hydroxymethyl)tetrahydropyran-3-yl]acetamide (XB48, 3.00 eq, 159 mg, 0.45 mmol) in DMSO (2.2 mL). The mixture was stirred at 45° C. overnight then purified by prep HPLC (5-12-30% MeCN/water with 0.1% formic acid) to give 4 as a white solid. Yield: 129 mg, 58%. LCMS m/z 1465.7 [M+H].

### Synthesis of N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (XB133)

To a mixture of benzyl (1,31-bis((2R,3R,4R,5R,6R)-3-acetamido-4,5-dihydroxy-6-(hydroxymethyl)tetrahydro-2H-pyran-2-yl)-16-(15-((2R,3R,4R,5R,6R)-3-acetamido-4,5-dihydroxy-6-(hydroxymethyl)tetrahydro-2H-pyran-2-yl)-5-oxo-9,12-dioxa-4,6-diazapentadecyl)-11,21-dioxo-4,7,25,28-tetraoxa-10,12,20,22-tetraazahentriacontan-16-yl)carbamate (4, 1.00 eq, 129 mg, 0.088 mmol) in MeOH (6 mL) was added 10% Pd/C (39 mg). The mixture was stirred at rt under hydrogen for 1 h, filtered, concentrated, and purified by prep HPLC (3-50% MeCN/20 mM NH₄OH aqueous solution) to give XB133 as a white solid. Yield: 104.2 mg, 89%. LCMS m/z 1331.7 [M+H].

### Synthesis of Compound 2313

To a mixture of 3-[2-[2-[2-[[4-(2,5-dioxopyrrol-1-yl)phenyl]sulfonylamino]ethoxy]ethoxy]ethoxy]propanoic acid (2, 1.10 eq, 3.7 mg, 0.0082 mmol) in DMF (0.25 mL) were added DIEA (3.00 eq, 0.0039 mL, 0.022 mmol), HATU (1.10 eq, 3.1 mg, 0.0082 mmol) and N-[(2R,3R,4R,5R,6R)-2-[3-[2-[2-[[7-[2-[2-[3-[(2R,3R,4R,5R,6R)-3- acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (Compound 2313, 1.00 eq, 9.9 mg, 0.0074 mmol). The mixture was stirred at rt for 30 minutes. To a solution of 3-[2-[2-[2-[[4-(2,5-dioxopyrrol-1-yl)phenyl]sulfonylamino]ethoxy]ethoxy]ethoxy]propanoic acid (0.68 mg) in DMF (0.1 mL) were added DIEA (1 uL) and HATU (0.6 mg). This mixture was added to the above reaction mixture. The mixture was stirred at rt for 30 minutes and purified by prep. HPLC (5-13-25% MeCN/water with 0.1% TFA) to give Compound 2313 as a white solid. Yield: 8.2 mg, 62%. LCMS m/z 1771.1 [M+H].

### Synthesis of 3a

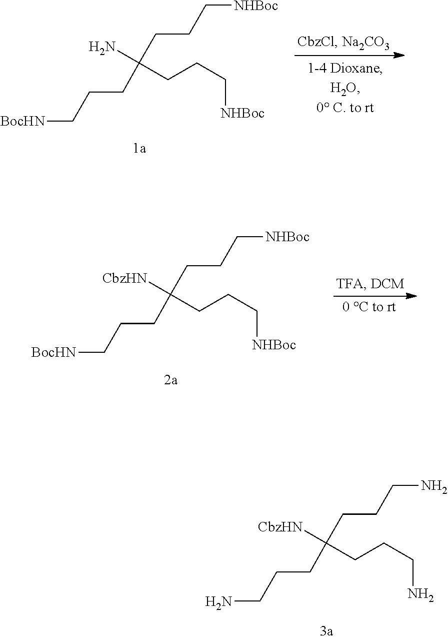 [SMILES: CC(C)(C)OC(=O)NCCCC(N)(CCCNC(=O)OC(C)(C)C)CCCNC(=O)OC(C)(C)C ; CC(C)(C)OC(=O)NCCCC(CCCNC(=O)OC(C)(C)C)(CCCNC(=O)OC(C)(C)C)NC(=O)OCc1ccccc1 ; NCCCC(CCCN)(CCCN)NC(=O)OCc1ccccc1]

### Synthesis of benzyl di-tert-butyl (4-(3-((tert-butoxycarbonyl)amino)propyl)heptane-1,4,7-triyl)tricarbamate (2a)

A solution of di-tert-butyl (4-amino-4-(3-((tert-butoxycarbonyl)amino)propyl)heptane-1,7-diyl)dicarbamate (1a, 1.0 eq, 1.1 g, 2.19 mmol) in 1,4-dioxane (9 mL) and water (3 mL) was cooled at 0° C. Sodium carbonate (3.0 eq, 0.70 g, 6.56 mmol) and benzyl chloroformate (50% solution in toluene) (1.5 eq, 1.12 mL, 3.28 mmol) were added and reaction mixture was stirred at room temperature for 16 h. After completion, water was added to the reaction mixture and product extracted with ethyl acetate. The organic layer was dried over anhydrous sodium sulfate, filtered and concentrated to get crude which was purified by column chromatography using silica gel (100-200 mesh, 0-15% ethyl acetate in hexane) to afford benzyl di-tert-butyl (4-(3-((tert-butoxycarbonyl)amino)propyl)heptane- 1,4,7-triyl)tricarbamate (2a) as a colorless viscous liquid. Yield: 1.3 g, 93.3%; LCMS m/z 637.55 [M+1]⁺.

### Synthesis of benzyl (1,7-diamino-4-(3-aminopropyl)heptan-4-yl)carbamate (3a)

A solution of benzyl di-tert-butyl (4-(3-((tert-butoxycarbonyl)amino)propyl)heptane-1,4,7-triyl)tricarbamate (1, 1.0 eq, 1.3 g, 2.04 mmol) in dichloromethane (13 mL) was cooled at 0° C. Trifluoroacetic acid (13 mL) was added and reaction mixture was stirred at room temperature for 1 h. After completion, the reaction mixture was concentrated, azeotroped with dichloromethane (2-3 times), dissolved in minimum amount of water, neutralized with ammonia solution (pH-8) and concentrated to get crude which was purified by prep HPLC (15-25% acetonitrile in water with 0.1% ammonium hydroxide) to afford 3a as an off white solid. Yield: 0.495 g, 72.1%; LCMS m/z 337.20 [M+1]+; 1H NMR (400 MHz, DMSO-d₆ with D₂O & d-TFA) δ 7.34-7.26 (m, 5H), 4.96 (s, 2H), 2.72 (t, J=6.8 Hz, 6H), 1.62-1.52 (m, 6H), 1.51-1.41 (m, 6H).

### Synthesis of Compound 2325

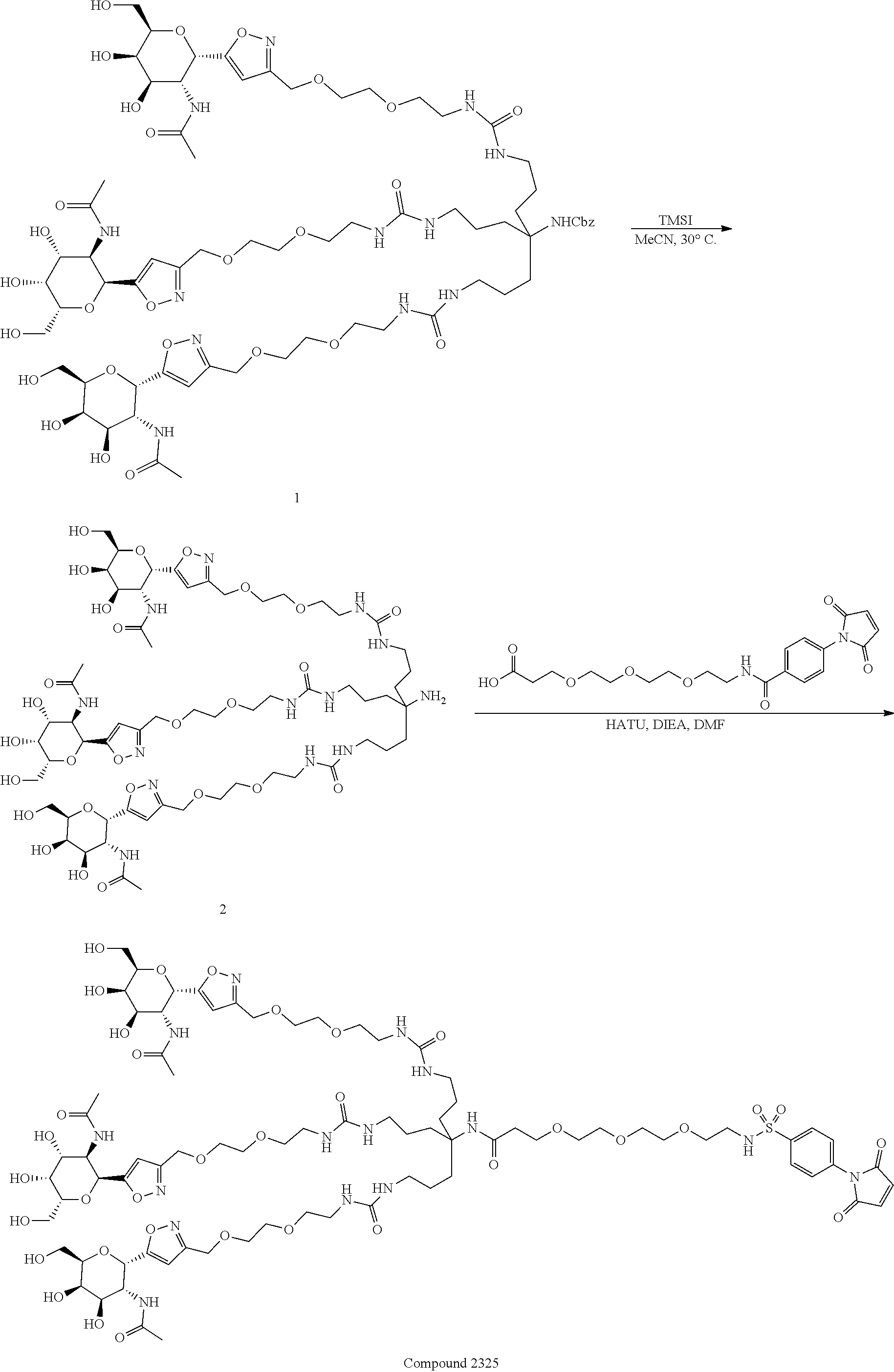

### Synthesis of benzyl (1,27-bis(5-((2S,3R,4R,5R,6R)-3-acetamido-4,5-dihydroxy-6-(hydroxymethyl)tetrahydro-2H-pyran-2-yl)isoxazol-3-yl)-14-(1-(5-((2S,3R,4R,5R,6R)-3-acetamido-4,5-dihydroxy-6-(hydroxymethyl)tetrahydro-2H-pyran-2-yl)isoxazol-3-yl)-9-oxo-2,5-dioxa-8,10 -diazatridecan-13-yl)-9,19-dioxo-2,5,23,26-tetraoxa-8,10,18,20-tetraazaheptacosan-14-yl)carbamate (1)

Compound 1 was synthesized according to General Method D using N-((2S,3R,4R,5R,6R)-2-(3-((2-(2-aminoethoxy)ethoxy)methyl)isoxazol-5-yl)-4,5-dihydroxy-6-(hydroxymethyl)tetrahydro-2H-pyran-3-yl)acetamide (XB86) in lieu of (XB48) to give intermediate (1). LCMS 1583.97 [M+H].

### Synthesis of N-[(2S,3R,4R,5R,6R)-2-[3-[2-[2-[[7-[2-[2-[[5-[(2S,3R,4R,5R,6R)-3-acetamido-4,5-dihydroxy-6-(hydroxymethyl)tetrahydropyran-2-yl]isoxazol-3-yl]methoxy]ethoxy]ethylcarbamoylamino]-4-[3-[2-[2-[[5-[(2S,3R,4R,5R,6R)-3-acetamido-4,5-dihydroxy-6-(hydroxymethyl)tetrahydropyran-2-yl]isoxazol-3-yl]methoxy]ethoxy]ethylcarbamoylamino]propyl]-4-amino-heptyl]carbamoylamino]ethoxy]ethoxymethyl]isoxazol-5-yl]-4,5-dihydroxy-6-(hydroxymethyl)tetrahydropyran-3-yl]acetamide (2)

Using air-free techniques, a mixture of 1 (1.00 eq, 9.0 mg, 0.00569 mmol) and MeCN (0.057 mL) was treated with iodo(trimethyl)silane (12.0 eq, 9.8 uL, 0.0682 mmol). More MeCN was added (0.200 mL) to enable stirring when a precipitate formed, then the reaction was heated to 30° C. After 1 h, the reaction was cooled to room temperature then diluted with concentrated ammonium hydroxide (0.050 mL) and DMSO (1 mL), then purified by reversed-phase HPLC (10-50% acetonitrile in water with 0.2 mM NH4OH modifier) to give (2) (5.0 mg, 0.00345 mmol, 60.7% yield). LCMS 1448.35 [M+H].

### Synthesis of Compound 2325

Following the HATU coupling method outlined in General Method D, 2 was converted to Compound 2325. LCMS 1888.25 [M+H].

### Synthesis of Compound 2314

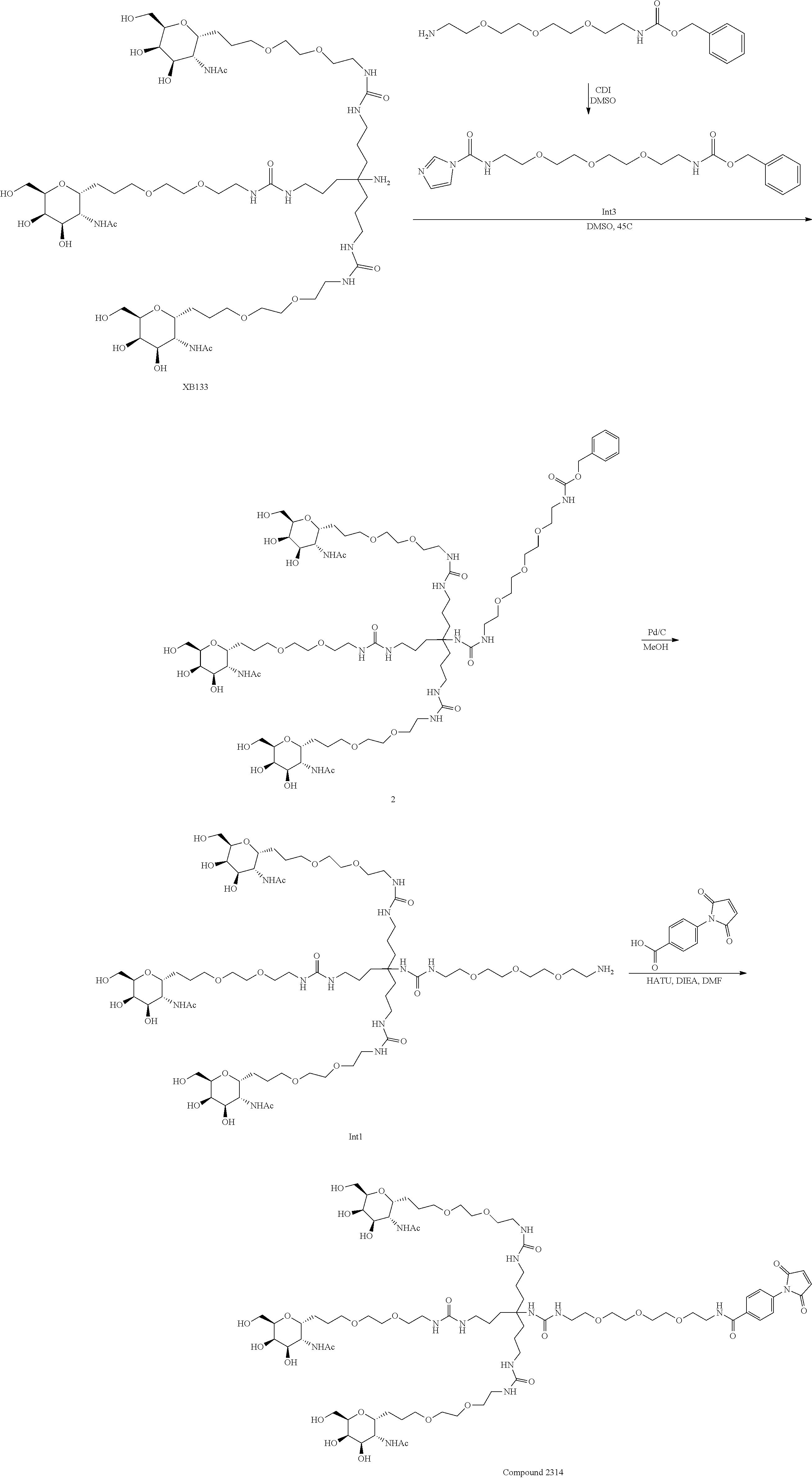

### Synthesis of benzyl N-[2-[2-[2-[2-(imidazole-1-carbonylamino)ethoxy]ethoxy]ethoxy]ethyl]carbamate (Int3)

To a mixture of Carbonyldiimidazole (1.10 eq, 115 mg, 0.71 mmol) in DMSO (0.4 mL) was added a solution of CbzNH-PEG3-CH₂CH₂NH₂ (1.00 eq, 210 mg, 0.64 mmol) in DMSO (1.2 mL) dropwise. The mixture was stirred at rt for 2h, diluted with EtOAc, washed with water (lx), and the aqueous layer was extracted with EtOAc. The combined organic layer was washed with water (lx) and brine (lx), dried, and concentrated, and purified by column (0-10% MeOH/DCM) to give Int3 as clear syrup (179 mg, yield: 66%). LCMS m/z 420.9 [M+H].

### Synthesis of benzyl (30-((2R,3R,4R,5R,6R)-3-acetamido-4,5-dihydroxy-6-(hydroxymethyl)tetrahydro-2H-pyran-2-yl)-15,15-bis(15-((2R,3R,4R,5R,6R)-3-acetamido-4,5-dihydroxy-6-(hydroxymethyl)tetrahydro-2H-pyran-2-yl)-5-oxo-9,12-dioxa-4,6-diazapentadecyl)-13,20-dioxo-3,6,9,24,27-pentaoxa-12,14,19,21-tetraazatriacontyl)carbamate (2)

To N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3 -yl]acetamide (XB133, 1.00 eq, 50.0 mg, 0.038 mmol) was added a solution of benzyl N-[2-[2-[2-[2-(imidazole-1-carbonylamino)ethoxy]ethoxy]ethoxy]ethyl]carbamate (Int3, 1.80 eq, 28.4 mg, 0.068 mmol) in DMSO (0.7 mL). The mixture was stirred at 45° C. overnight and purified by prep. HPLC (5-12-30% MeCN/water with 0.1% formic acid), and re-purified by prep. HPLC (5-12-30% MeCN/water with 0.1% TFA) to give 2 as a white solid (45 mg, yield: 71%). LCMS m/z 1683.8 [M+H].

### Synthesis of Intermediate Int1

To a mixture of benzyl (30-((2R,3R,4R,5R,6R)-3-acetamido-4,5-dihydroxy-6-(hydroxymethyl)tetrahydro-2H-pyran-2-yl)-15,15-bis(15-((2R,3R,4R,5R,6R)-3-acetamido-4,5-dihydroxy-6-(hydroxymethyl)tetrahydro-2H-pyran-2-yl)-5-oxo-9,12-dioxa-4,6-diazapentadecyl)-13,20-dioxo-3,6,9,24,27-pentaoxa-12,14,19,21-tetraazatriacontyl)carbamate (2, 1.00 eq, 45.0 mg, 0.027 mmol) in MeOH (4 mL) was added 10% Pd/C (14 mg). The mixture was stirred at rt under hydrogen for 1 h, filtered, concentrated, and purified by prep. HPLC (3-50% MeCN/20 mM NH₄OH solution) to give Int1 as a white solid (31.9 mg, yield: 77%). LCMS m/z 1549.8 [M+H].

### Synthesis of Compound 2314

To a mixture of 4-Maleimidobenzoic acid (1.15 eq, 2.2 mg, 0.010 mmol) in DMF (0.1 mL) were added DIPEA (3.00 eq, 0.0046 mL, 0.026 mmol) and HATU (1.15 eq, 3.8 mg, 0.010 mmol), and a solution of intermediate Int1 (1.00 eq, 13.6 mg, 0.0088 mmol) in DMF (0.2 mL). The mixture was stirred at rt for 30 minutes and purified by prep. HPLC (3-14-27% MeCN/water with 0.1% TFA) to give Compound 2314 as a white solid (12.8 mg, yield: 83%). LCMS m/z 1749.5 [M+H].

### Synthesis of Compound 2316

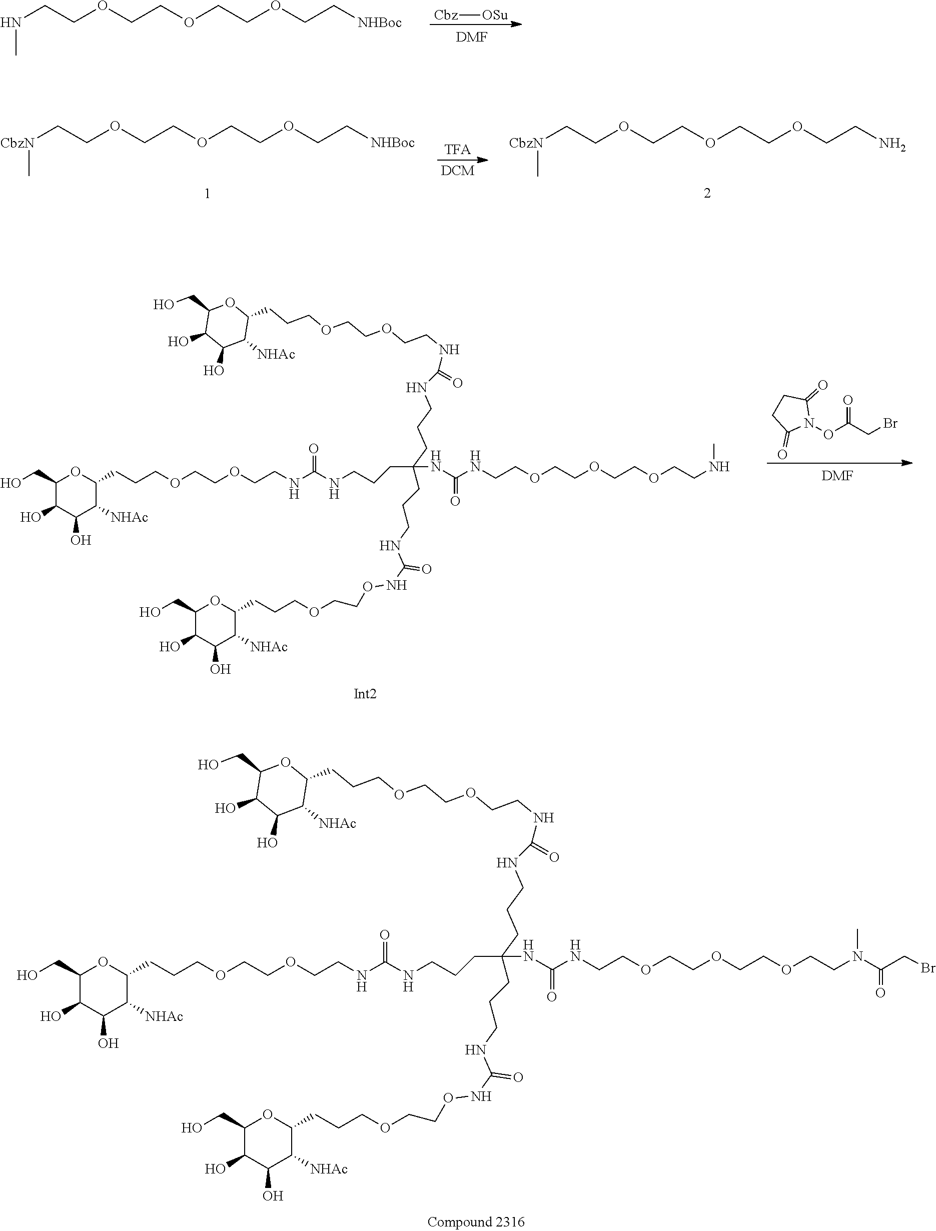

### Synthesis of benzyl N-[2-[2-[2-[2-(tert-butoxycarbonylamino)ethoxy]ethoxy]ethoxy]ethyl]-N-methyl-carbamate (1)

To a mixture of tert-butyl N-[2-[2-[2-[2-(methylamino)ethoxy]ethoxy]ethoxy]ethyl]carbamate (1.00 eq, 198 mg, 0.65 mmol) in DMF (0.4 mL) was added a solution of benzyl 2,5-dioxopyrrolidin-1-yl carbonate (1.30 eq, 210 mg, 0.84 mmol) in DMF (1 mL). The mixture was stirred at rt for 2 h and purified by prep. HPLC (20-100% MeCN/water with 0.1% formic acid) to give 1 as clear syrup (192 mg, yield: 67%). LCMS 341.3 [M-Boc+H]⁺.

### Synthesis of benzyl N-[2-[2-[2-(2-aminoethoxy)ethoxy]ethoxy]ethyl]-N-methyl-carbamate (2)

To a mixture of benzyl N-[2-[2-[2-[2-(tert-butoxycarbonylamino)ethoxy]ethoxy]ethoxy]ethyl]-N-methyl-carbamate (1, 1.00 eq, 282 mg, 0.64 mmol) in DCM (1 mL) was added TFA (1 mL). The mixture was stirred at rt for 1 h and concentrated. The residue was basified with sat. NaHCO₃ and extracted with DCM (3×). The combined organic layer was dried, concentrated to give 2 as yellow syrup (230 mg, yield: 105%). LCMS m/z 341.2 [M+H].

### Synthesis of intermediate Int2

Intermediate Int2 was synthesized by employing the procedures described for intermediate Compound 2321-Int1 using benzyl N-[2-[2-[2-(2-aminoethoxy)ethoxy]ethoxy]ethyl]-N-methyl-carbamate (2) in lieu of CbzNH-PEG3-CH₂CH₂NH₂. LCMS m/z 1564.8 [M+H].

### Synthesis of Compound 2316

Compound 2316 was synthesized by employing the procedures described for Compound 2321 using intermediate Int2 in lieu of intermediate Compound 2321-Int1. LCMS m/z 1685.7 [M+H].

### Synthesis of Compound 2321

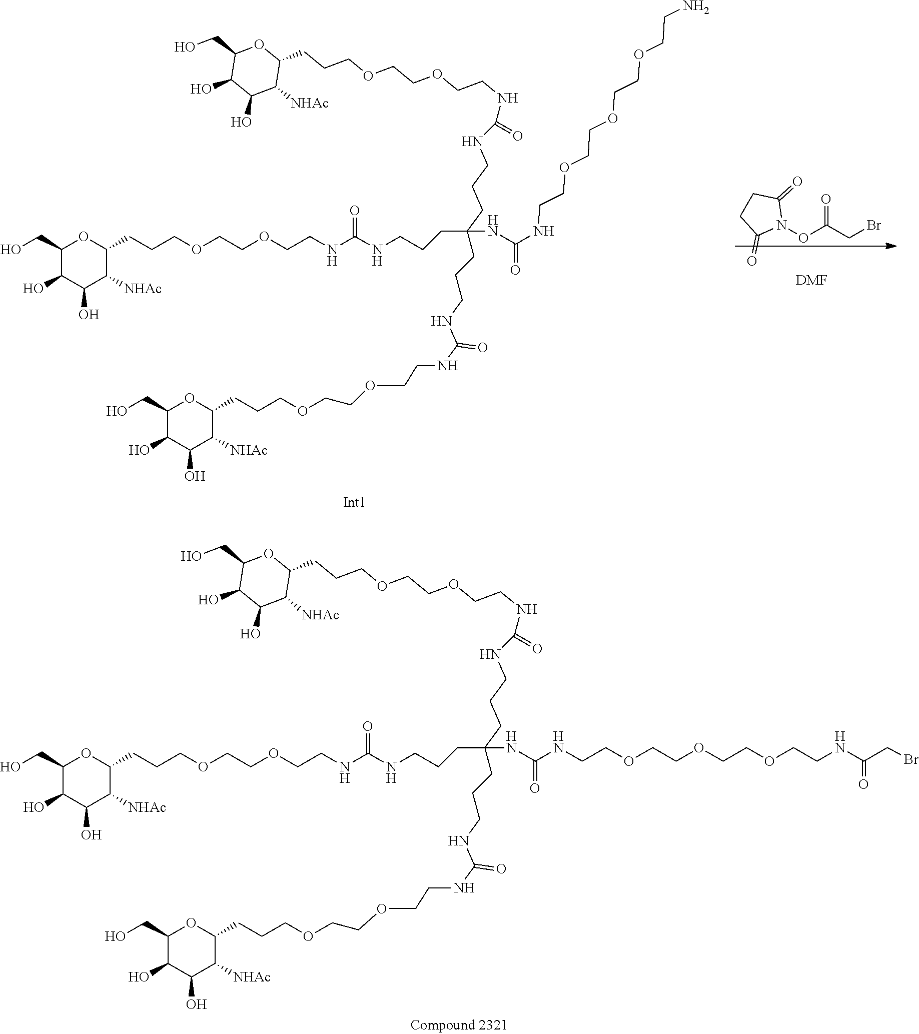

### Synthesis of Compound 2321

To a mixture of 2,5-dioxypyrrolidin-1-ul 2-bromoacetate (1.49 eq, 2.3 mg, 0.0098 mmol) in DMF (0.2 mL) at 0° C. was added a mixture of Int1 (1.00 eq, 10.2 mg, 0.0066 mmol) in DMF (0.3 mL). The mixture was stirred at 0° C. for 30 minutes and purified by prep. HPLC (3-10-24% with 0.1% formic acid) and re-purified by prep. HPLC (3-10-24% MeCN/water with 0.1% TFA) to give Compound 2321 as a white solid (5.5 mg, yield: 50%). LCMS: 1671.7 [M+H].

### Synthesis of Compound 2315

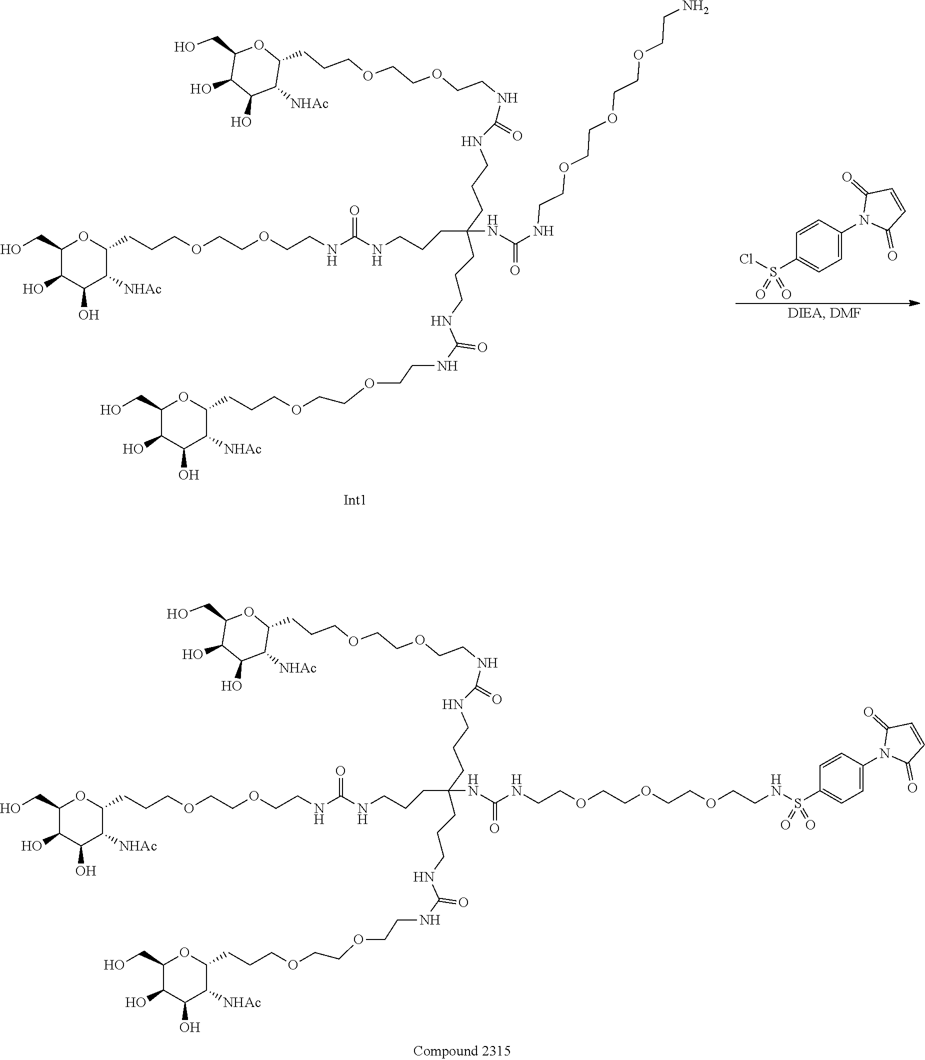

### Synthesis of Compound 2315

To a mixture of 4-(2,5-dioxopyrrol-1-yl)benzenesulfonyl chloride (1.05 eq, 1.9 mg, 0.0068 mmol) in DMF (0.05 mL) at 0° C. were added DIPEA (1.40 eq, 0.0016 mL, 0.0091 mmol) and a mixture of Int1 (1.00 eq, 10.1 mg, 0.0065 mmol) in DMF (0.2 mL). The mixture was stirred at 0° C. for 20 minutes and purified by prep. HPLC (3-13-25% MeCN/water with 0.1% formic acid) to give Compound 2315 as a white solid (7.9 mg, yield: 68%). LCMS 1785.8 [M+H].

### Synthesis of Compound 2318

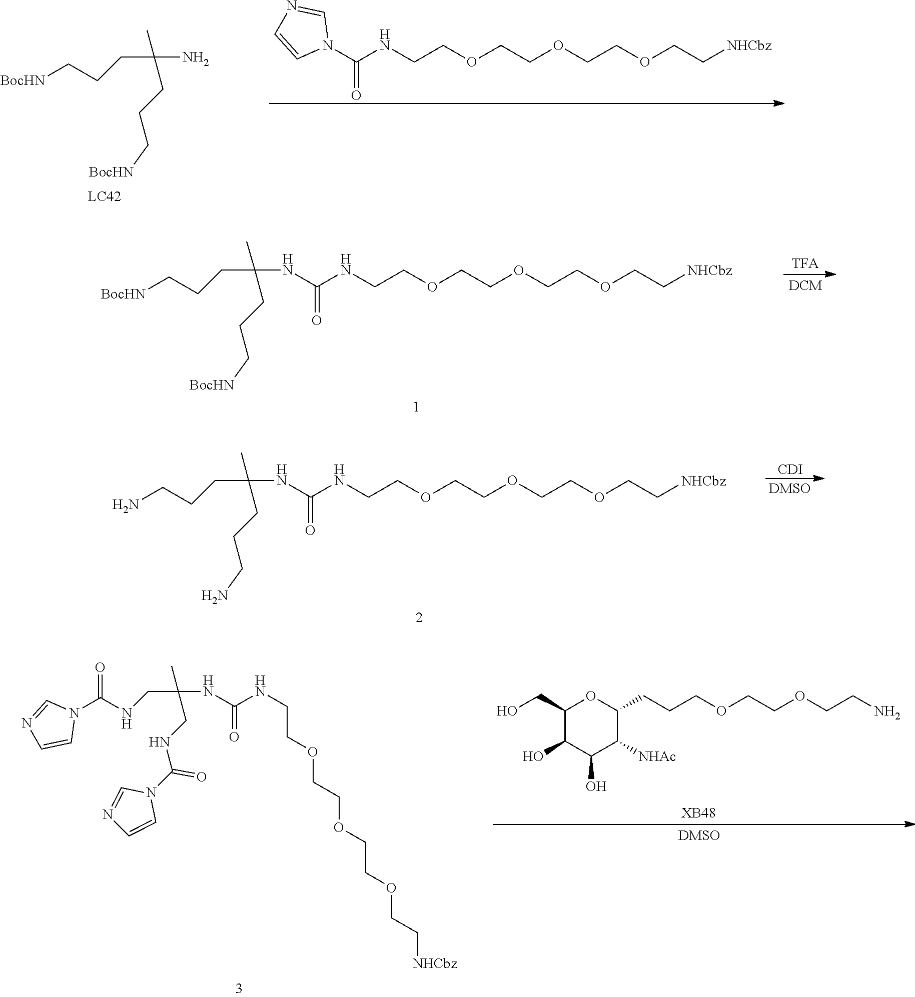

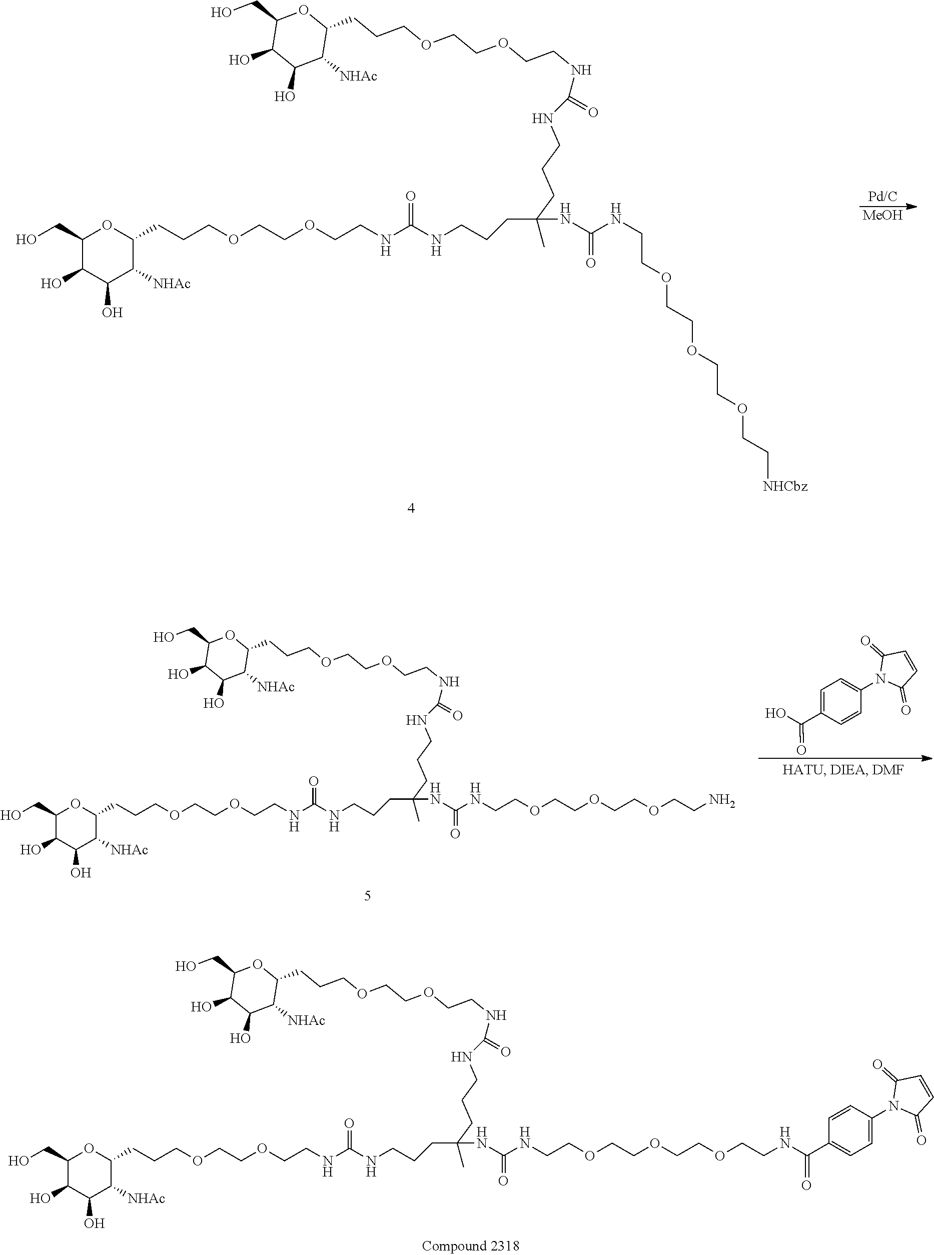

### Synthesis of tert-butyl N-[4-[2-[2-[2-[2-(benzyloxycarbonylamino)ethoxy]ethoxy]ethoxy]ethylcarbamoylamino]-7-(tert-butoxycarbonylamino)-4-methyl-heptyl]carbamate (1)

To a mixture of benzyl N-[2-[2-[2-[2-(imidazole-1-carbonylamino)ethoxy]ethoxy]ethoxy]ethyl]carbamate (Int3, 1.46 eq, 51.2 mg, 0.12 mmol) in DMSO (0.25 mL) was added tert-butyl N-[4-amino-7-(tert-butoxycarbonylamino)-4-methyl-heptyl]carbamate (L C42, 1.00 eq, 30.0 mg, 0.083 mmol) and DIPEA (1.00 eq, 0.015 mL, 0.083 mmol). The mixture was stirred at 50° C. overnight and at 60° C. for 5 h and purified by prep. HPLC (20c- 80% MeCN/water with 0.1% formic acid) to give las clear syrup (41.6 mg, yield: 70% o). LCMS m/z 712.2 [M+H].

### Synthesis of benzyl N-[2-[2-[2-[2-[[4-amino-1-(3-aminopropyl)-1-methyl-butyl]carbamoylamino]ethoxy]ethoxy]ethoxy]ethyl]carbamate; 2,2,2-trifluoroacetic acid (2)

To a mixture of tert-butyl N-[4-[2-[2-[2-[2-(benzyloxycarbonylamino)ethoxy]ethoxy]ethoxy]ethylcarbamoylamino]-7-(tert-butoxycarbonylamino)-4-methyl-heptyl]carbamate (1, 1.00 eq, 63.7 mg, 0.089 mmol) in DCM (1 mL) was added TFA (1 mL). The mixture was stirred at rt for 1h, concentrated, and lyophilized to give 2 as clear syrup (83 mg, yield: 108%). LCMS m/z 512.2 [M+H].

### Synthesis of benzyl N-[2-[2-[2-[2-[[4-(imidazole-1-carbonylamino)-1-[3-(imidazole-1-carbonylamino)propyl]-1-methyl-butyl]carbamoylamino]ethoxy]ethoxy]ethoxy]ethyl]carbamate (3)

To a mixture of carbonyldiimidazole (5.80 eq, 91.4 mg, 0.56 mmol) in DMSO (0.2 mL) was added a solution of benzyl N-[2-[2-[2-[2-[[4-amino-1-(3-aminopropyl)-1-methyl-butyl]carbamoylamino]ethoxy]ethoxy]ethoxy]ethyl]carbamate; 2,2,2-trifluoroacetic acid (2, 1.00 eq, 83.0 mg, 0.097 mmol) in DMSO (0.8 mL). The mixture was stirred at rt for 2h, diluted with EtOAc, washed with water (lx), and the aqueous layer was extracted with EtOAc (lx). The combined organic layer was washed with water (lx) and brine (lx), dried, and concentrated to give 3 as clear syrup (59.5 mg, yield: 87%). LCMS m/z 700.2 [M+H].

### Synthesis of benzyl N-[2-[2-[2-[2-[[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-1-methyl-butyl]carbamoylamino]ethoxy]ethoxy]ethoxy]ethyl]carbamate (4)

To a mixture of benzyl N-[2-[2-[2-[2-[[4-(imidazole-1-carbonylamino)-1-[3-(imidazole-1-carbonylamino)propyl]-1-methyl-butyl]carbamoylamino]ethoxy]ethoxy]ethoxy]ethyl]carbamate (3, 1.00 eq, 32.4 mg, 0.046 mmol) in DMSO (0.5 mL) was added N-[(2R,3R,4R,5R,6R)-2-[3-[2-(2-aminoethoxy)ethoxy]propyl]-4,5-dihydroxy-6-(hydroxymethyl)tetrahydropyran-3-yl]acetamide (XB48 2.10 eq, 34.1 mg, 0.097 mmol). The mixture was stirred at rt over weekend, purified by prep. HPLC (5-35% MeCN/water with 0.1% formic acid) to give 4 as a white solid (39.5 mg, yield: 67%). LCMS 1264.5 [M+H].

### Synthesis of N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[2-[2-[2-(2-aminoethoxy)ethoxy]ethoxy]ethylcarbamoylamino]-4-methyl-heptyl]carbamoylamino]ethoxy]ethoxy]propyl]-4,5-dihydroxy-6-(hydroxymethyl)tetrahydropyran-3-yl]acetamide (5)

To a mixture of benzyl N-[2-[2-[2-[2-[[4-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-1-[3-[2-[2-[3-[(2R,3R,4R,5R,6R)-3-acetamido-4,5-dihydroxy-6-(hydroxymethyl)tetrahydropyran-2 -yl]propoxy]ethoxy]ethylcarbamoylamino]propyl]-1-methyl-butyl]carbamoylamino]ethoxy]ethoxy]ethoxy]ethyl]carbamate (4, 1.00 eq, 39.5 mg, 0.031 mmol) in MeOH (3 mL) was added 10% Pd/C (12 mg). The mixture was stirred at rt under hydrogen for 1 h, filtered, concentrated, purified by prep. HPLC (5-50% MeCN/20 mM NH4OH solution) to give 5 as a white solid (24.6 mg, yield: 70%). LCMS 1130.6 [M+H].

### Synthesis of N,N′-((2R,2′R,3R,3′R,4R,4′R,5R,5′R,6R,6′R)-(16-(3-(1-(4-(2,5-dioxo-2,5-dihydro-1H-pyrrol-1-yl)phenyl)-1-oxo-5,8,11-trioxa-2-azatridecan-13-yl)ureido)-16-methyl-11,21-dioxo-4,7,25,28-tetraoxa-10,12,20,22-tetraazahentriacontane-1,31-diyl)bis(4,5-dihydroxy-6-(hydroxymethyl)tetrahydro-2H-pyran-2,3-diyl))diacetamide (Compound 2318)

To a mixture of 4-Maleimidobenzoic acid (1.10 eq, 1.8 mg, 0.0081 mmol) in DMF (0.3 mL) were added DIPEA (3.00 eq, 0.0038 mL, 0.022 mmol), HATU (1.10 eq, 3.1 mg, 0.0081 mmol), and N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[2-[2-[2-(2-aminoethoxy)ethoxy]ethoxy]ethylcarbamoylamino]-4-methyl-heptyl]carbamoylamino]ethoxy]ethoxy]propyl]-4,5-dihydroxy-6-(hydroxymethyl)tetrahydropyran-3-yl]acetamide (5, 1.00 eq, 8.3 mg, 0.0073 mmol). The mixture was stirred at rt for 1 h and purified by prep. HPLC (10-25% MeCN/water with 0.1% formic acid) to give Compound 2318 as a white solid (5.9 mg, yield: 60%). LCMS 1329.5 [M+H].

### Synthesis of Compound 2319-C

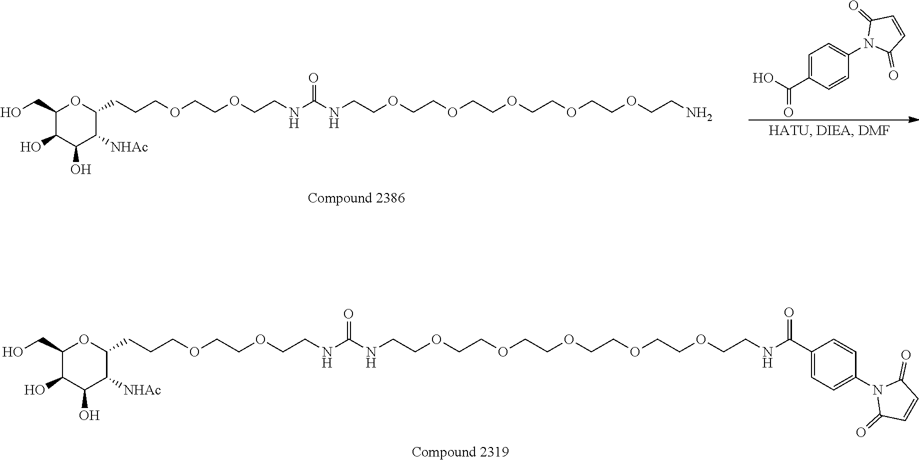 [SMILES: CC(=O)N[C@@H]1[C@@H](O)[C@@H](O)[C@@H](CO)O[C@@H]1CCCOCCOCCNC(=O)NCCOCCOCCOCCOCCOCCN ; O=C(O)c1ccc(N2C(=O)C=CC2=O)cc1 ; CC(=O)N[C@@H]1[C@@H](O)[C@@H](O)[C@@H](CO)O[C@@H]1CCCOCCOCCNC(=O)NCCOCCOCCOCCOCCOCCNC(=O)c1ccc(N2C(=O)C=CC2=O)cc1]

### Synthesis of N-(29-((2R,3R,4R,5R,6R)-3-acetamido-4,5-dihydroxy-6-(hydroxymethyl)tetrahydro-2H-pyran-2-yl)-19-oxo-3,6,9,12,15,23,26-heptaoxa-18,20-diazanonacosyl)-4-(2,5-dioxo-2,5-dihydro-1H-pyrrol-1-yl)benzamide (Compound 2319-C)

To a mixture of 4-maleimidobenzoic acid (1.10 eq, 3.6 mg, 0.0165 mmol) in DMF (0.1 mL) were added DIPEA (3.00 eq, 0.0078 mL, 0.0450 mmol), HATU (1.10 eq, 6.3 mg, 0.0165 mmol), and a solution of N-[(2R,3R,4R,5R,6R)-2-[3-[2-[2-[2-[2-[2-[2-[2-(2-aminoethoxy)ethoxy]ethoxy]ethoxy]ethoxy]ethylcarbamoylamino]ethoxy]ethoxy]propyl]-4,5-dihydroxy-6-(hydroxymethyl)tetrahydropyran-3-yl]acetamide (Compound 2386, 1.00 eq, 9.9 mg, 0.0150 mmol) in DMF (0.2 mL). The mixture was stirred at rt for 1 h and purified by prep. HPLC (10- 25% MeCN/water with 0.1% formic acid) to give Compound 2319-C as a white solid (9 mg, yield: 70%). LCMS m/z 856.4 [M+H].

### Synthesis of Compound 2317

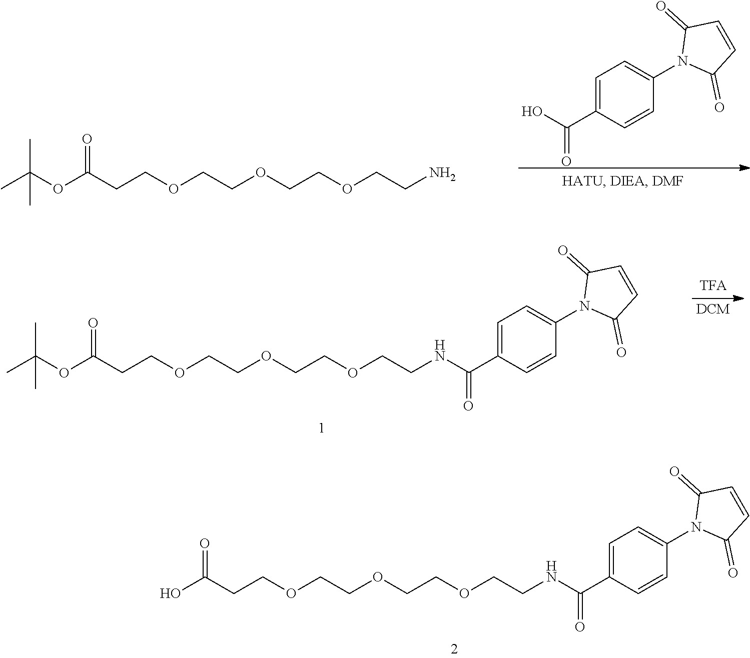 [SMILES: CC(C)(C)OC(=O)CCOCCOCCOCCN ; O=C(O)c1ccc(N2C(=O)C=CC2=O)cc1 ; CC(C)(C)OC(=O)CCOCCOCCOCCNC(=O)c1ccc(N2C(=O)C=CC2=O)cc1 ; O=C(O)CCOCCOCCOCCNC(=O)c1ccc(N2C(=O)C=CC2=O)cc1]

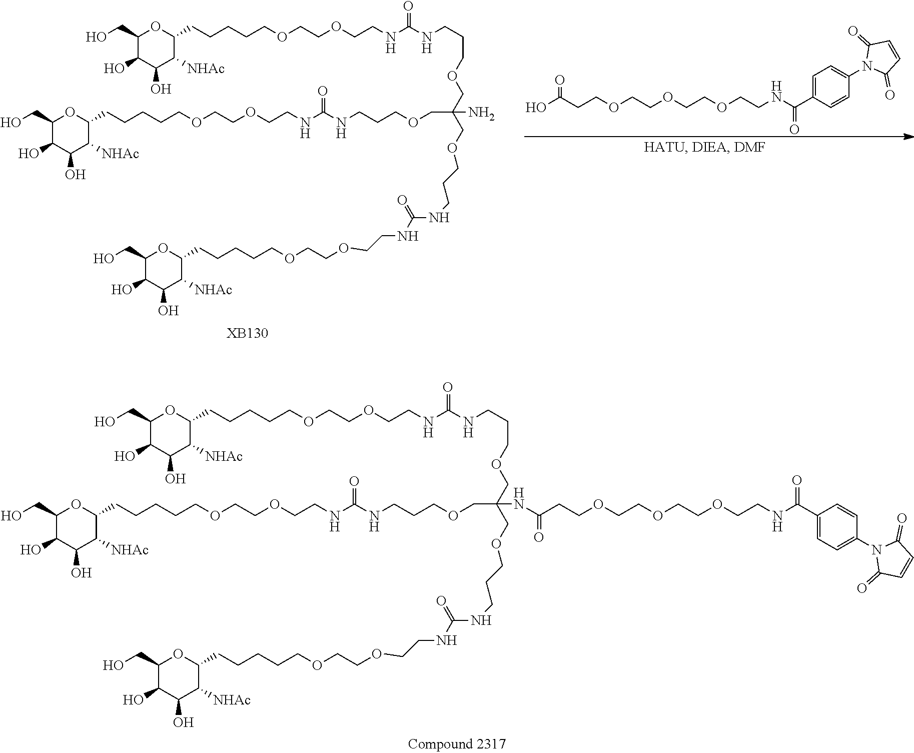

### Synthesis of tert-butyl 3-[2-[2-[2-[[4-(2,5-dioxopyrrol-1-yl)benzoyl]amino]ethoxy]ethoxy]ethoxy]propanoate (1)

To a mixture of 4-Maleimidobenzoic acid (1.00 eq, 102 mg, 0.47 mmol) in DMF (0.6 mL) at 0° C. were added HATU (1.10 eq, 196 mg, 0.52 mmol) and Diisopropylethylamine (DIPEA) (2.00 eq, 0.16 mL, 0.94 mmol) and a solution of tert-butyl 3-(2-(2-(2-aminoethoxy)ethoxy)ethoxy)propanoate (1.00 eq, 130 mg, 0.47 mmol) in DMF (0.4 mL). The mixture was stirred at 0° C. for 20 minutes and was purified by prep. HPLC (10-60% MeCN/water with 0.1% TFA) to give 1 as yellow syrup (211 mg, yield: 95%). LCMS 477.6 [M+H].

### Synthesis of 3-[2-[2-[2-[[4-(2,5-dioxopyrrol-1-yl)benzoyl]amino]ethoxy]ethoxy]ethoxy]propanoic acid (2)

To a mixture of tert-butyl 3-[2-[2-[2-[[4-(2,5-dioxopyrrol-1-yl)benzoyl]amino]ethoxy]ethoxy]ethoxy]propanoate (1, 1.00 eq, 211 mg, 0.44 mmol) in DCM (1 mL) was added TFA (1 mL). The mixture was stirred at rt for 30 minutes, concentrated, and purified by prep. HPLC (10-70% MeCN/water) to give 2 as clear syrup (103 mg, yield: 55%). LCMS: 421.5 [M+H].

### Synthesis of N-[(2R,3R,4R,5R,6R)-2-[5-[2-[2-[3-[3-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxy]-2-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxymethyl]-2-amino-propoxy]propylcarbamoylamino]ethoxy]ethoxy]pentyl]-4,5-dihydroxy-6-(hydroxymethyl)tetrahydropyran-3-yl]acetamide (XB130)

Intermediate XB130 was synthesized by employing the procedures described for intermediate XB133 using benzyl (1,3-bis(3-aminopropoxy)-2-((3-aminopropoxy)methyl)propan-2-yl)carbamate; 3TFA salt in lieu of benzyl N-[4-amino-1,1-bis(3-aminopropyl)butyl]carbamate; 3TFA salt and using N-((2R,3R,4R,5R,6R)-2-(5-(2-(2-aminoethoxy)ethoxy)pentyl)-4,5-dihydroxy-6-(hydroxymethyl)tetrahydro-2H-pyran-3-yl)acetamide (XB47) in lieu of N-[(2R,3R,4R,5R,6R)-2-[3-[2-(2-aminoethoxy)ethoxy]propyl]-4,5-dihydroxy-6-(hydroxymethyl)tetrahydropyran-3-yl]acetamide (XB48). LCMS m/z 1505.5 [M+H].

### Synthesis of Compound 2317

Compound 2317 was synthesized by employing the procedures described for Compound 2313 using 3-[2-[2-[2-[[4-(2,5-dioxopyrrol-1-yl)benzoyl]amino]ethoxy]ethoxy]ethoxy]propanoic acid (2) in lieu of 3-(2-(2-(2-((4-(2,5-dioxo-2,5-dihydro-1H-pyrrol-1-yl)phenyl)sulfonamido)ethoxy)ethoxy)ethoxy)propanoic acid and using N-[(2R,3R,4R,5R,6R)-2-[5-[2-[2-[3-[3-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxy]-2-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxymethyl]-2-amino-propoxy]propylcarbamoylamino]ethoxy]ethoxy]pentyl]-4,5-dihydroxy-6-(hydroxymethyl)tetrahydropyran-3-yl]acetamide (XB130) in lieu of N-[(2R,3R,4R,5R,6R)-2-[3-[2-[2-[[7-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]-4-[3-[2-[2-[3-[(2R,3R,4R,5R,6R)-3-acetamido-4,5-dihydroxy-6-(hydroxymethyl)tetrahydropyran-2-yl]propoxy]ethoxy]ethylcarbamoylamino]propyl]-4-amino-heptyl]carbamoylamino]ethoxy]ethoxy]propyl]-4,5-dihydroxy-6-(hydroxymethyl)tetrahydropyran-3-yl]acetamide (XB133). LCMS m/z 1908.5 [M+H].

### Method E: Synthesis of Compound 2305 from XB84

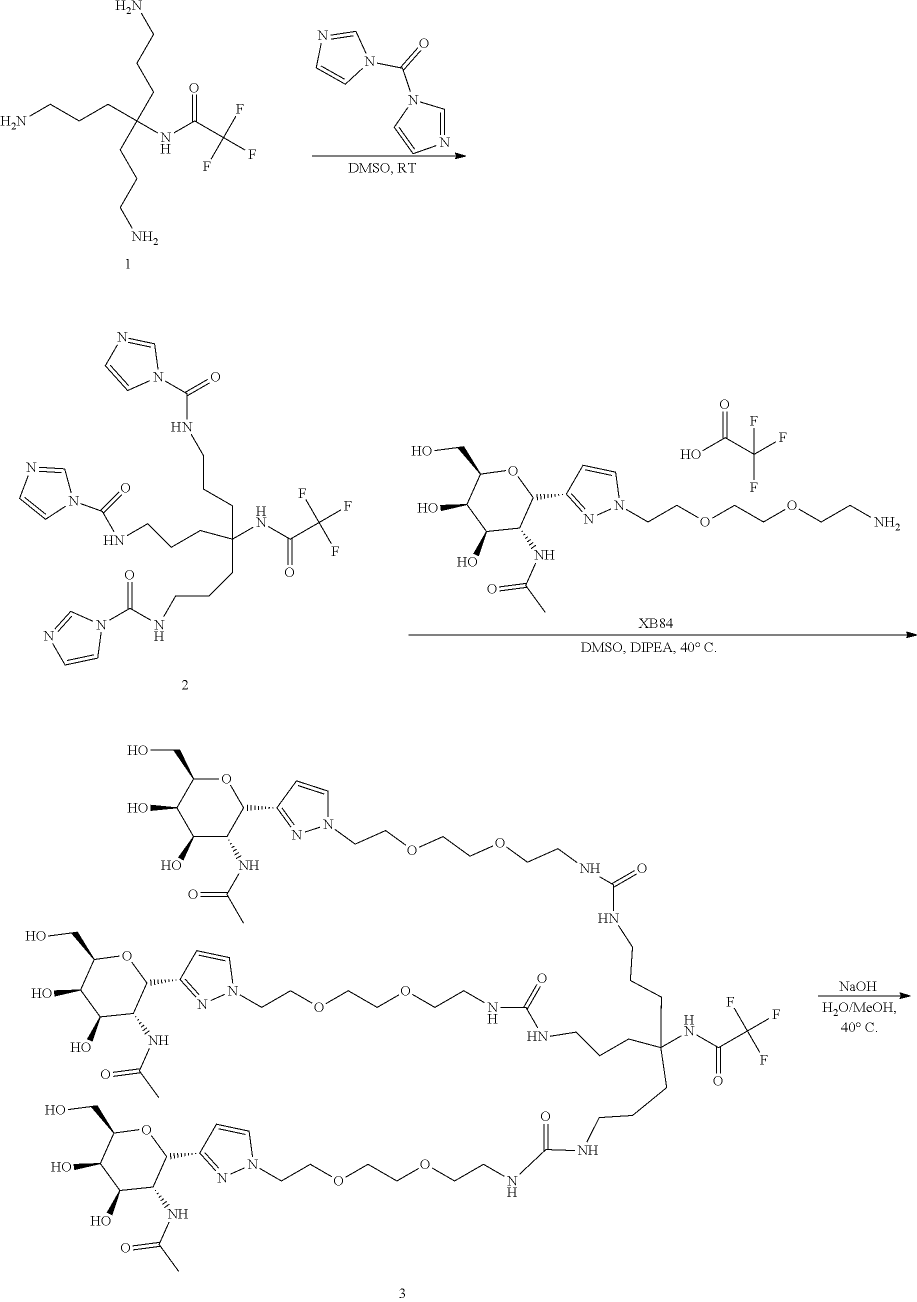

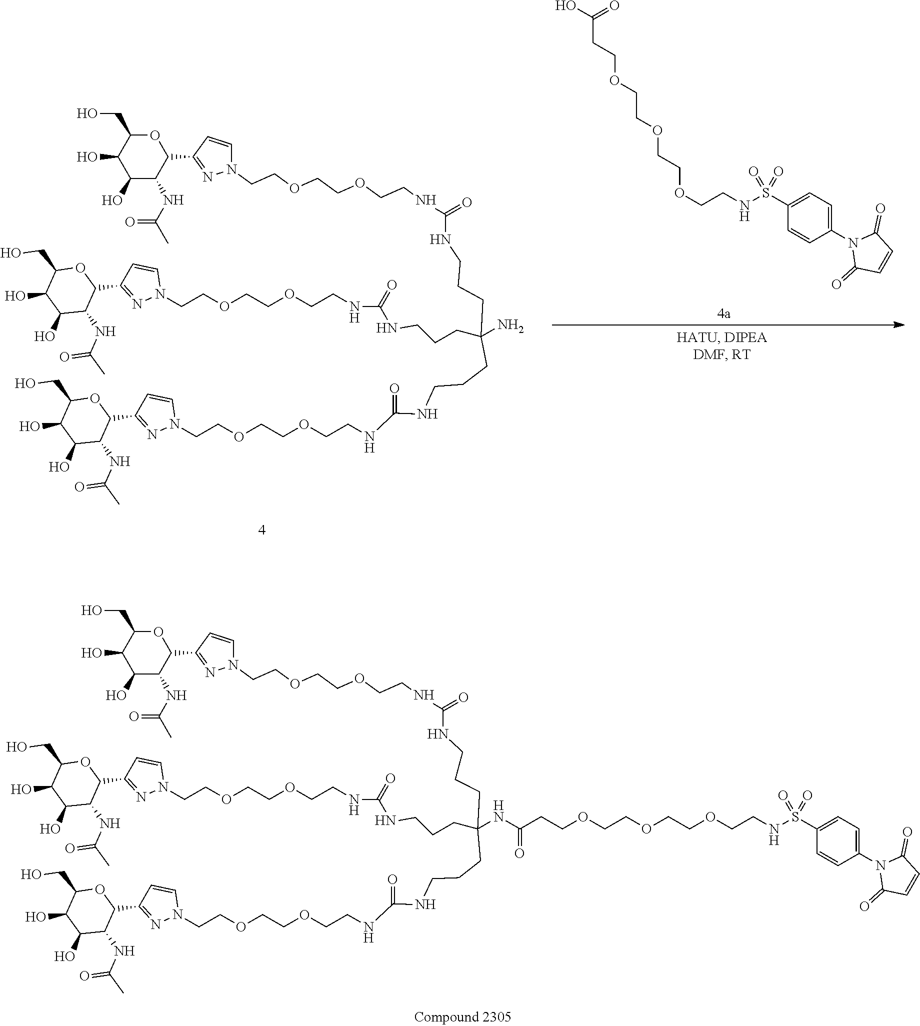

### Synthesis of N,N′-(4-(3-(1H-imidazole-1-carboxamido)propyl)-4-(2,2,2-trifluoroacetamido)heptane-1,7-diyl)bis(1H-imidazole-1-carboxamide) (2)

To a solution of N-[4-amino-1,1-bis(3-aminopropyl)butyl]-2,2,2-trifluoro-acetamide; 2,2,2-trifluoroacetic acid (1) (57.0 mg, 0.0890 mmol, 1.0 eq.) in 0.6 mL DMSO was added carbonyldiimidazole (108.2 mg, 0.668 mmol, 7.5 eq). The reaction solution was stirred under nitrogen atmosphere and ambient temperature for 1 hr. The reaction mixture was diluted with 1 mL of DMSO and purified by reverse phase HPLC (5-100% acetonitrile in water). Fractions containing desired product were combined and lyophilized to dryness to afford N-[7-(imidazole-1-carbonylamino)-4-[3-(imidazole-1-carbonylamino)propyl]-4-[(2,2,2-trifluoroacetyl)amino]heptyl]imidazole-1-carboxamide (2) as a white solid. Yield: 55 mg, 106%; LCMS m/z 581.04 [M+H].

### Synthesis of N-[4-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]-1,1-bis[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]butyl]-2,2,2-trifluoro-acetamide (3)

To a stirring solution of N-[(2S,3R,4R,5R,6R)-2-[1-[2-[2-(2-aminoethoxy)ethoxy]ethyl]pyrazol-3-yl]-4,5-dihydroxy-6-(hydroxymethyl)tetrahydropyran-3 -yl]acetamide; 2,2,2-trifluoroacetic acid (XB84) (21.7 mg, 0.0419 mmol, 3.1 eq.) in 0.2 mL of DMSO and diisopropylethylamine (DIPEA) (14.3 mL, 0.0821 mmol, 6.0 eq.) was added a solution of N-[7-(imidazole-1-carbonylamino)-4-[3-(imidazole-1-carbonylamino)propyl]-4-[(2,2,2-trifluoroacetyl)amino]heptyl]imidazole-1-carboxamide (2) (7.9 mg, 0.014 mmol, 1.0 eq) in 0.1 mL of DMSO. The reaction solution was stirred under a nitrogen atmosphere at 40° C. overnight, then was cooled to ambient temperature, diluted with 1 mL of DMSO, and purified by reverse phase HPLC (1-40% acetonitrile in water with 0.1% TFA modifier). Fractions containing desired product were combined and lyophilized to dryness to afford N-[4-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]-1,1-bis[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]butyl]-2,2,2-trifluoro-acetamide (3) as a white solid. Yield: 8.7 mg, 40%; LCMS m/z 1583.6 [M+H].

### Synthesis of N-[(2S,3R,4R,5R,6R)-2-[1-[2-[2-[2-[[7-[2-[2-[2-[3-[(2S,3R,4R,5R,6R)- 3-acetamido-4,5-dihydroxy-6-(hydroxymethyl)tetrahydropyran-2-yl]pyrazol-1-yl]ethoxy]ethoxy]ethylcarbamoylamino]-4-[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]-4-amino-heptyl]carbamoylamino]ethoxy]ethoxy]ethyl]pyrazol-3-yl]-4,5-dihydroxy-6-(hydroxymethyl)tetrahydropyran-3-yl]acetamide (4)

To a solution of N-[4-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]-1,1-bis[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]butyl]-2,2,2-trifluoro-acetamide (3) (8.7 mg, 0.0055 mmol, 1.0 eq.) in 0.9 mL of methanol was added 0.3 mL of aqueous 5N sodium hydroxide. The reaction stirred at 40° C. overnight, then the solution was concentrated to -¼ original volume under a stream of nitrogen. The concentrated crude mixture was diluted with 2 mL of H₂O and purified by reverse phase HPLC (2-40% acetonitrile in water with 20 mM ammonium hydroxide modifier). Fractions containing desired product were combined and lyophilized to dryness to afford N-[(2S,3R,4R,5R,6R)-2-[1-[2-[2-[2-[[7-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]-4-[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]-4-amino-heptyl]carbamoylamino]ethoxy]ethoxy]ethyl]pyrazol-3-yl]-4,5-dihydroxy-6-(hydroxymethyl)tetrahydropyran-3-yl]acetamide (4) as a white solid. Yield: 5.1 mg, 63%; LCMS m/z 1488.4 [M+H].

### Synthesis of Compound 2305

A solution of 3-[2-[2-[2-[[4-(2,5-dioxopyrrol-1-yl)phenyl]sulfonylamino]ethoxy]ethoxy]ethoxy]propanoic acid (4a) (2.3 mg, 0.0051 mmol, 1.5 eq.) in 0.1 mL of DMF was added HATU (2.1 mg, 0.0056 mmol, 1.7 eq.) and DIPEA (4.0 mL, 0.023 mmol, 7.0 eq.). The solution stirred under nitrogen atmosphere at ambient temperature for ~5 min, then was added to a stirring solution of N-[(2S,3R,4R,5R,6R)-2-[1-[2-[2-[2-[[7-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]-4-[3-[2-[2-[2-[3-[(2S,3R,4R,5R,6R)-3-acetamido-4,5-dihydroxy-6-(hydroxymethyl)tetrahydropyran-2-yl]pyrazol-1-yl]ethoxy]ethoxy]ethylcarbamoylamino]propyl]-4-amino-heptyl]carbamoylamino]ethoxy]ethoxy]ethyl]pyrazol-3-yl]-4,5-dihydroxy-6-(hydroxymethyl)tetrahydropyran-3-yl]acetamide (4) (4.9 mg, 0.0033 mmol, 1.0 eq) dissolved in 1.0 mL of DMF. The reaction solution stirred under nitrogen atmosphere at ambient temperature for 1 hr, then was diluted with 1 mL of DMSO and purified by reverse phase HPLC (5-100% acetonitrile in water with 0.1% TFA modifier). Fractions containing desired product were combined and lyophilized to dryness to afford Compound 2305 as a white solid. Yield: 4.3 mg, 67%; LCMS m/z 1926.5 [M+H].

### Synthesis of Compound 1-163

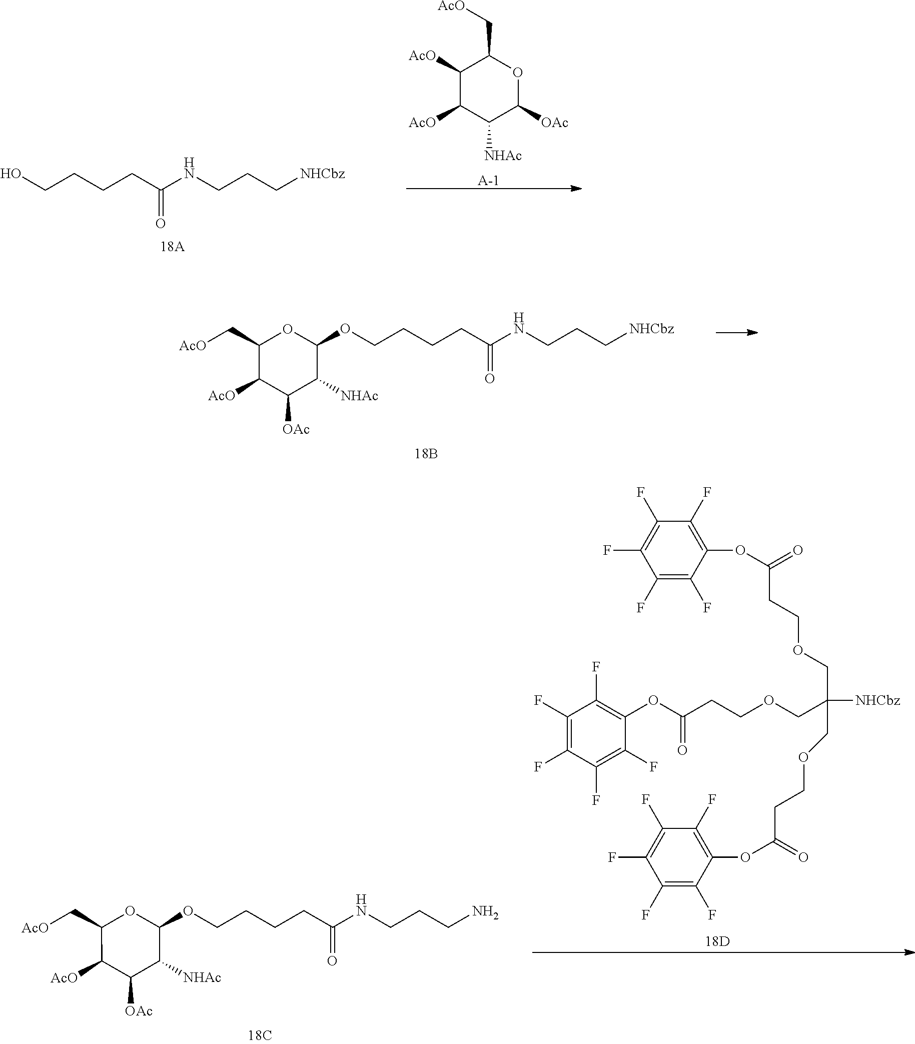

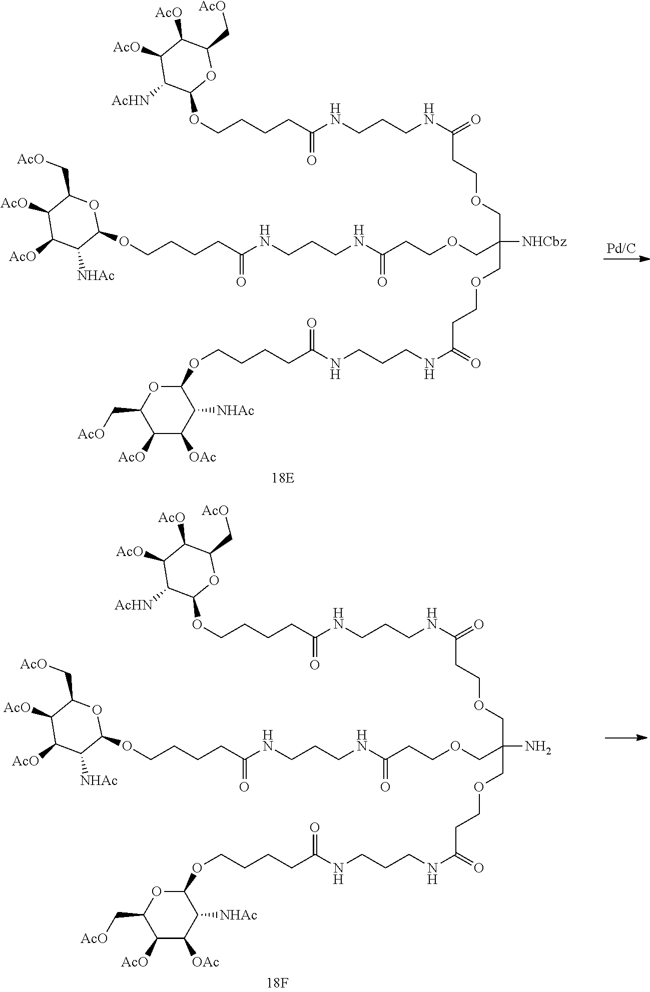

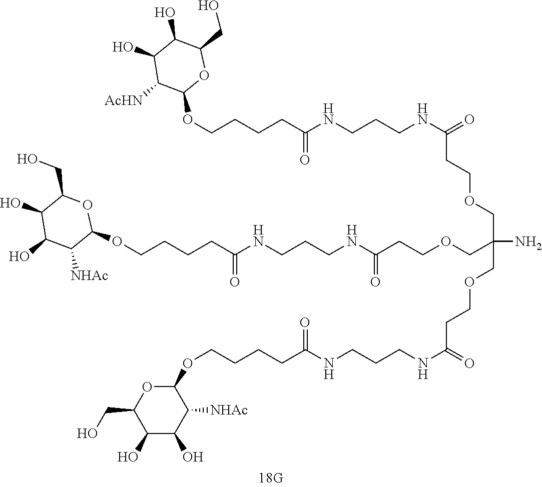 [SMILES: CC(=O)N[C@H]1[C@H](OCCCCC(=O)NCCCNC(=O)CCOCC(N)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

To the solution of Compound A-1 (1.0 eq, 5.05 g, 13.0 mmol) and benzyl N-[3-(5-hydroxypentanamido) propyl]carbamate (Compound 18A) (1.0 eq, 4.00 g, 13.0 mmol) in dichloromethane (50.0 mL), trimethylsilyl trifluoromethanesulfonate (1.1 eq, 2.52 mL, 14.3 mmol) was added dropwise at room temperature. The reaction mixture was stirred at 40° C. for 5 h. After completion, the reaction mixture was quenched with saturated sodium bicarbonate solution and extracted with dichloromethane. The organic layer was dried over sodium sulfate, filtered, and concentrated under high vacuum to get crude. The crude was purified by reverse phase chromatography using 0-30% acetonitrile in water to afford Compound 18B as yellow viscous liquid, Yield: (5.80 g, 70.12%); LCMS m/z 638.2 [M+1]+

To a solution of Compound 18B (1.0 eq, 4.80 g, 7.53 mmol) in methanol (40.0 mL), 10% Palladium on carbon (1.60 g) was added and stirred at room temperature under hydrogen atmosphere for 4 h. After completion, the reaction mixture was filtered through syringe filter, filtrate was concentrated and dried to get crude. The crude was triturated with diethyl ether to afford Compound 18C as a pale yellow viscous liquid. Yield: (3.4 g, 80.73%); LCMS m/z 504.37 [M+1]⁺.

A solution of 2,3,4,5,6-pentafluorophenyl 3-(2-{[(benzyloxy)carbonyl]amino}-3-[3-oxo-3-(2,3,4,5,6-pentafluorophenoxy)propoxy]-2-{[3-oxo-3-(2,3,4,5,6-pentafluorophenoxy)propoxy]methyl}propoxy)propanoate (18D) (1.0 eq, 1.20 g, 1.24 mmol) and Compound 18C (3.0 eq, 1.87 g, 3.71 mmol) in N,N-dimethylformamide (30.0 mL) was stirred at room temperature for 1 h. After completion, the reaction mixture was concentrated and dried to get crude. The crude was purified by flash column chromatography using 20% methanol in dichloromethane to afford Compound 18E as pale yellow viscous liquid. Yield: (1.60 g; 67.05%); LCMS m/z 1926.78 [M−1]⁻.

To a solution of Compound 18E (1.0 eq, 1.60 g, 0.830 mmol) in methanol (20 mL) and acetic acid (1.0 mL), 10% Palladium on carbon (250 mg) was added. The reaction mixture was stirred at room temperature under hydrogen atmosphere for 16 h. After completion, the reaction mixture was filtered through celite bed, filtrate was concentrated and dried to afford Compound 18F as pale yellow viscous liquid. Yield: 1.45 g (Crude); LCMS m/z 1794.05 [M+1]⁺.

To a solution of Compound 18F (1.0 eq, 1.45 g, 0.808 mmol) in methanol (10 mL), 25% sodium methanolate solution (8.0 eq, 1.45 mL, 6.47 mmol) was added at 0° C. The reaction mixture was stirred at room temperature for 1 h. After completion reaction, reaction mixture was concentrated and dry to get crude. The crude was diluted with acetonitrile and purified by preparative HPLC (30% acetonitrile in water with 0.1% TFA). Fractions containing the desired product were combined and lyophilized to dryness to afford Compound 18G as an off white semi solid. Yield: (0.20 g, 17.4%); LCMS m/z 1415.77 [M+1]⁺.

### Synthesis of Compound I-124

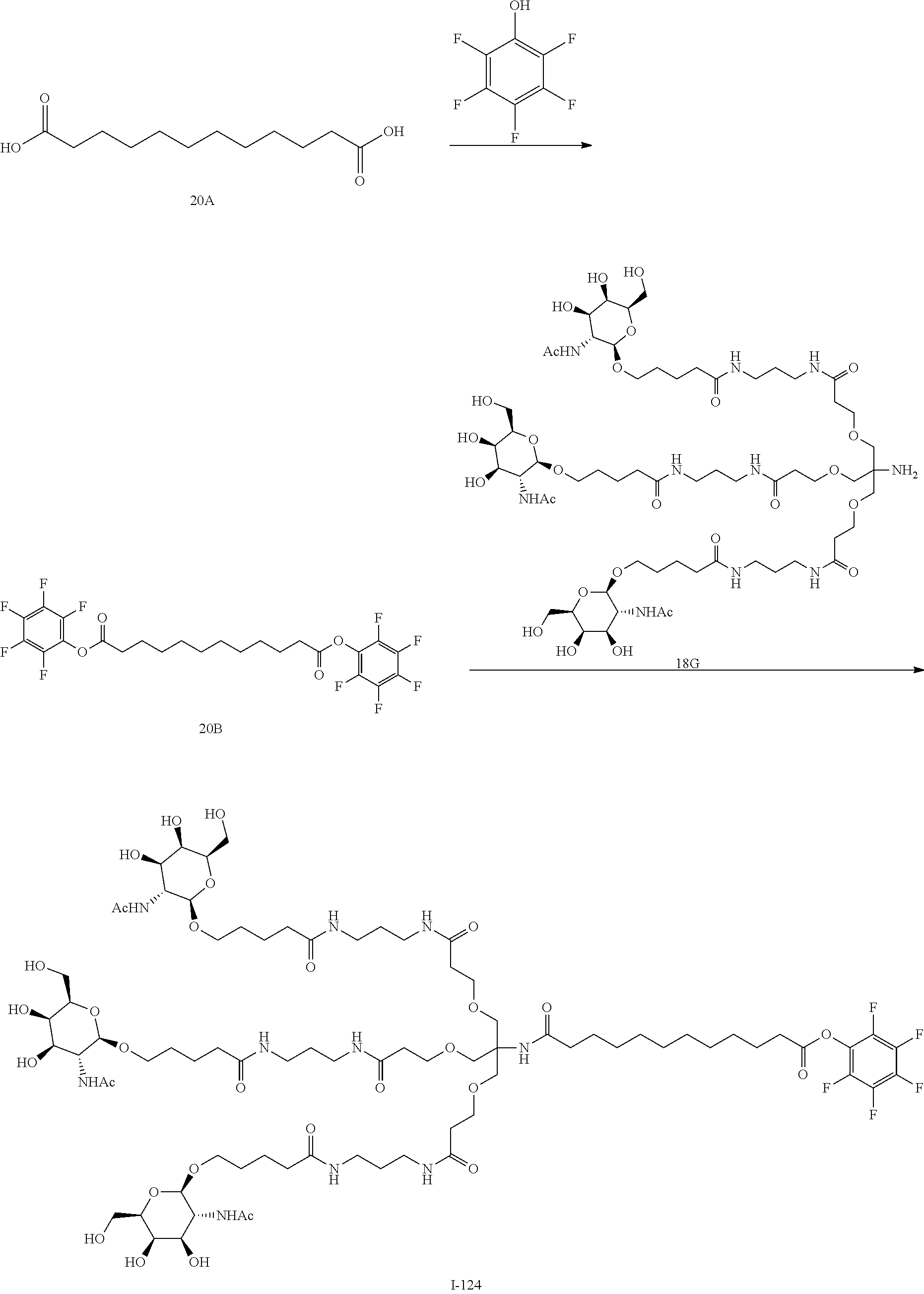

To the solution of dodecanedioic acid (20A) (1.00 g, 4.34 mmol) in ethyl acetate (10.00 mL) at 0° C., pentafluorophenol (1.60 g, 8.68 mmol) and diisopropylmethanediimine (1.91 mL, 13.0 mmol) were added and reaction mixture stirred at room temperature for 1 h. After completion, reaction mixture was filtered through celite bed and filtrate was concentrated under reduced pressure to get crude compound. Crude compound obtained was purified by flash column chromatography on silica gel column using 5% ethyl acetate in hexanes as eluents to afford Compound 20B as off white solid. Yield: 1.00 g (40.95%); LCMS m/z 580.39 [M+18]⁺.

To a solution Compound 18G (45.0 mg, 0.031 mmol) in dimethyl sulfoxide (1.0 mL) was added N,N-diisopropylethylamine (0.016 mL, 0.093 mmol) and Compound 20B (17.9 mg, 0.031 mmol). Reaction mixture was stirred at room temperature for 2 h. After completion, the reaction mixture was purified via preparatory HPLC (40-60% acetonitrile in water with 0.1% trifluoroacetic acid). Fractions containing the desired product were combined and lyophilized to dryness to afford Compound I-124 as an off white solid. Yield: 0.006 g (10.52%); LCMS m/z 1793.94 [M+1]+, 897.99 [M/2+1]. ¹H NMR (400 MHz, DMSO-d6) δ 7.83 (t, J=5.6 Hz, 3H), 7.73 (t, J=5.2 Hz, 3H), 7.60 (d, J=9.2 Hz, 3H), 6.99 (s, 1H), 4.57-4.47 (m, 6H), 4.46 (d, J=4.4 Hz, 3H), 4.21 (d, J=8.4 Hz, 3H), 3.70-3.63 (m, 9H), 3.55-3.49 (m, 21H), 3.32-3.28 (m, 4H), 3.02 (t, J=5.6 Hz, 12H), 2.76 (t, J=5.6 Hz, 2H), 2.27 (t, J=6.4 Hz, 6H), 2.03 (t, J=7.2 Hz, 8H), 1.79 (s, 9H), 1.70-1.67 (m, 2H), 1.52-1.41 (m, 20H), 1.23 (bs, 14H).

### Synthesis of Compound I-163

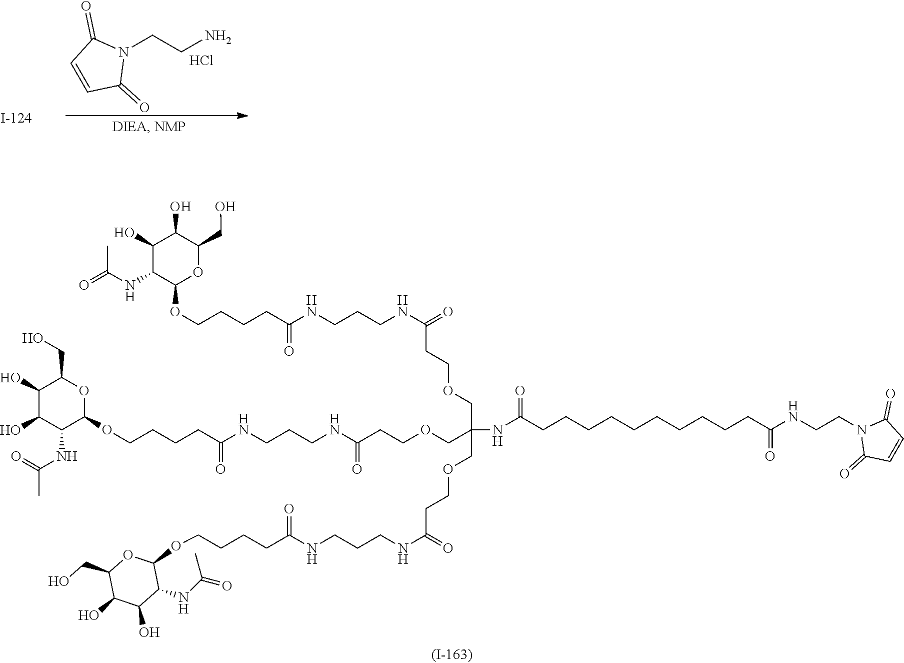 [SMILES: NCCN1C(=O)C=CC1=O ; Cl ; CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O]

To a mixture of Compound I-124 (1 eq, 17.2 mg. 0.00959 mmol) and 1-(2-aminoethyl)pyrrole-2,5-dione; hydrochloride (1.1 eq, 1.86 mg, 0.00105 mmol) in NMP (0.5 mL) was added DIEA (3 eq, 5 μL, 0.0288 mmol). The mixture was stirred at rt for 10 minutes and acetic acid (4 uL) was added. The mixture was purified by prep. HPLC (10-40% MeCN/water with 0.1% TFA) to give compound I-163 as a white solid (10.1 mg, yield 64%). (purity. 99%). LCMS m/z 1751.0 [M+H]+.

### Synthesis of Compound 2402C

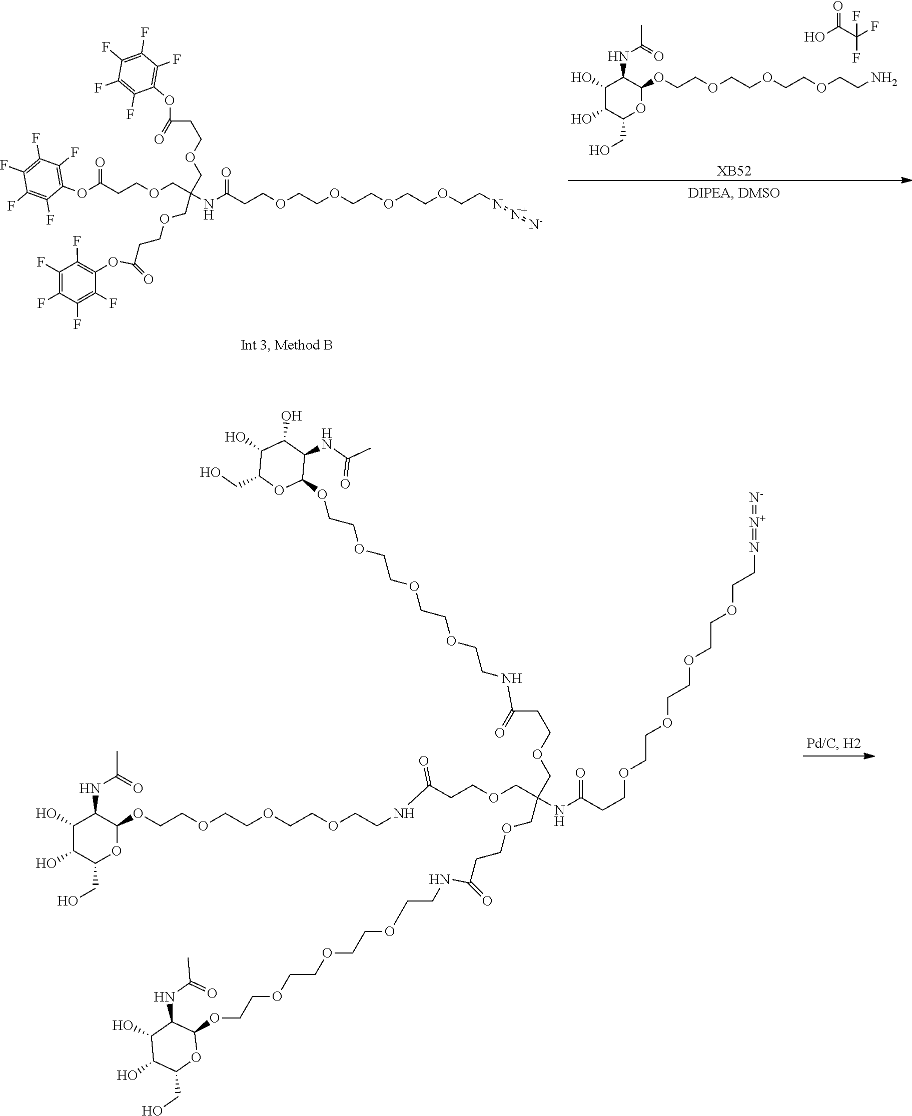

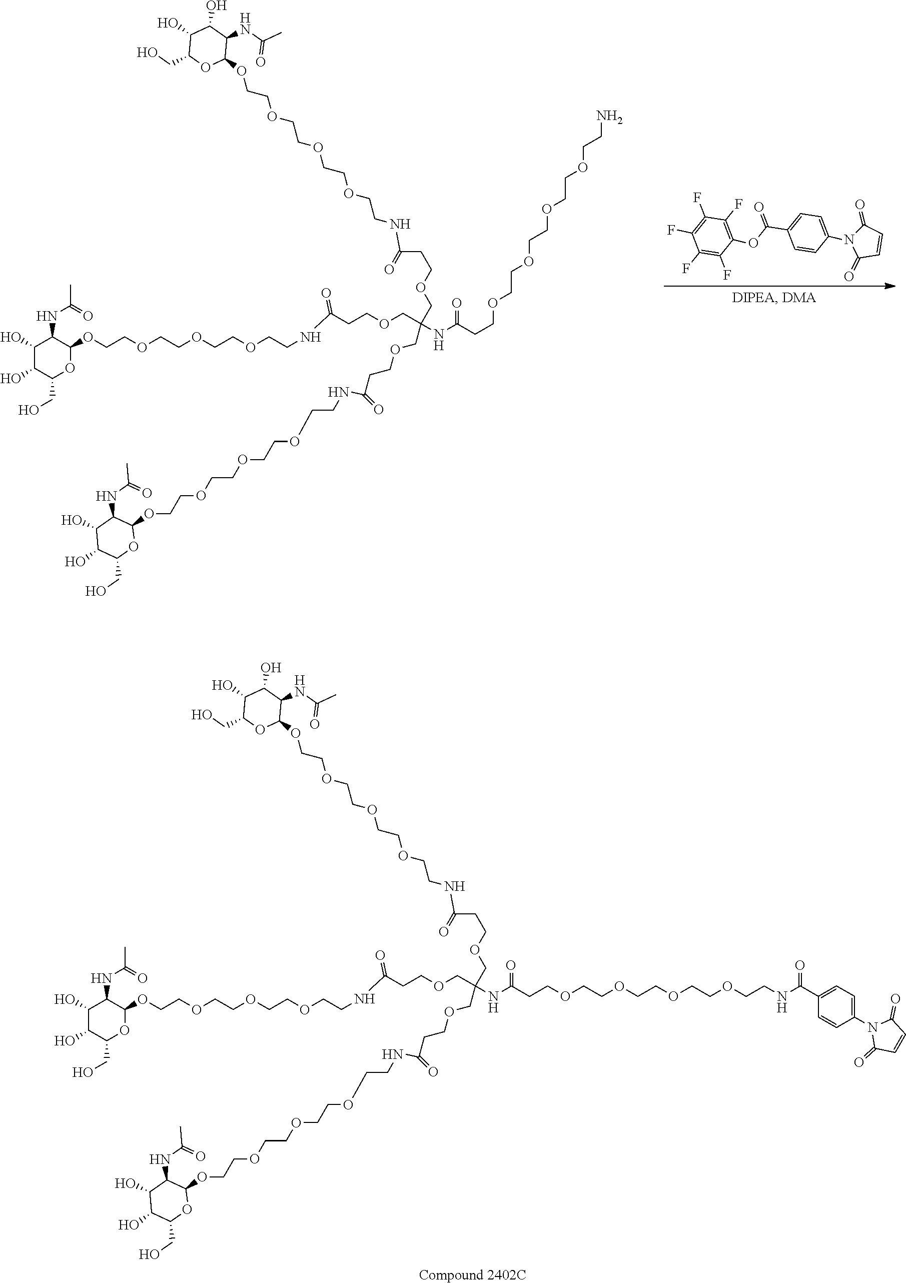

N-[2-[2-[2-[2-[3-[[2-[3-[2-[2-[2-[2-[(2S,3R,4R,5R,6R)-3-acetamido-4,5-dihydroxy-6-(hydroxymethyl)tetrahydropyran-2-yl]oxyethoxy]ethoxy]ethoxy]ethylamino]-3-oxo-propoxy]-1,1-bis[[3-[2-[2-[2-[2-[(2S,3R,4R,5R,6R)-3-acetamido-4,5-dihydroxy-6-(hydroxymethyl)tetrahydropyran-2-yl]oxyethoxy]ethoxy]ethoxy]ethylamino]-3-oxo-propoxy]methyl]ethyl]amino]-3-oxo-propoxy]ethoxy]ethoxy]ethoxy]ethyl]-4-(2,5-dioxopyrrol-1-yl)benzamide (Compound 2402C) was synthesized analogous to General Synthesis Method B, starting with XB52 amine TFA salt: LCMS m/z 1919.5 [M+H].

### Synthesis of Compound 1248: General Synthesis Method A

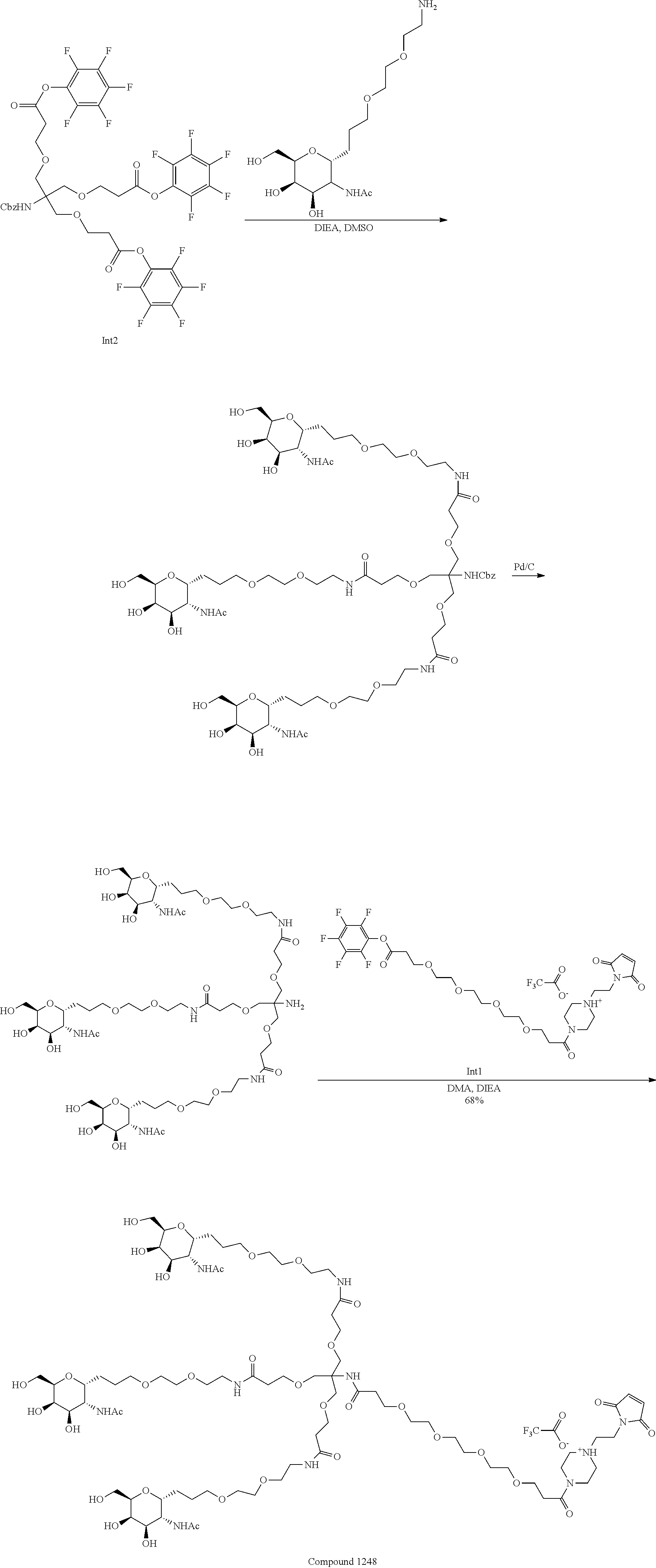

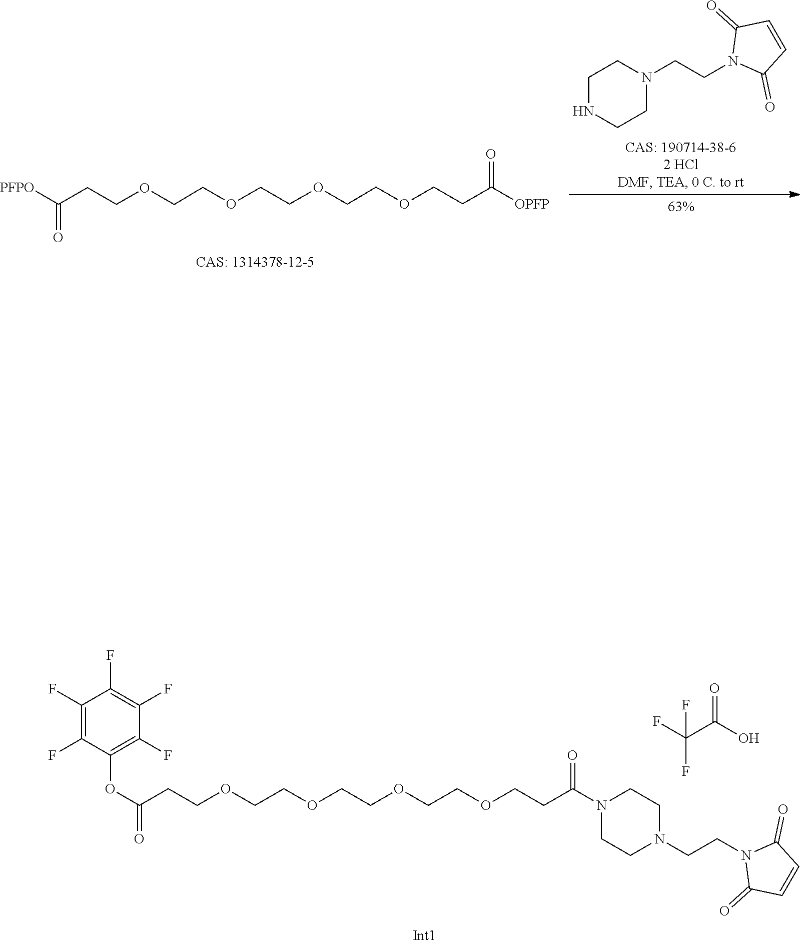

### Synthesis of perfluorophenyl 16-(4-(2-(2,5-dioxo-2,5-dihydro-1H-pyrrol-1-yl)ethyl)piperazin-1-yl)-16-oxo-4,7,10,13-tetraoxahexadecanoate, TFA salt (Int1)

To a suspension of 1-(2-piperazin-1-ylethyl)pyrrole-2,5-dione; dihydrochloride (1.00 eq, 202 mg, 0.716 mmol) in 1.5 mL of DMF was added Bis-PEG4-PFP ester (1.50 eq, 673 mg, 1.07 mmol) in 1 mL DMF, the mixture was cooled to 0° C. and triethylamine (3.00 eq, 0.30 mL, 2.15 mmol) was added dropwise - mixture remains cloudy, partial slurry - stirred at 0° C. for 20 min and then let warm to room temp and stirred for 30 min. The mixture was cooled to 0° C., acidified with 165 uL TFA (3 eq) in approx. 0.50 mL water, diluted with CH₃CN, filtered and purified by preparative HPLC on C18 column (30×250 mm) eluting with a gradient of 15-75-100% CH₃CN/water+0.1% TFA. Collected 343 mg of the desired product as a clear oil after lyophilization of fractions, 63% yield. HPLC: 100% by ELSD, >99% @ 254 nm [5-99 CH₃CN/water+0.1% TFA over 8 min., 0.9 mL/min, Agilent-poroshell 120, C18, 2.7 um, 50×3 mm] LCMS 652.5 [M+H], [10-100 CH₃CN/water+0.1% FA over 7 min., 0.9 ml/min, Agilent, poroshell120, C18, 2.1 mm×50 mm, 2.7 um]; 1H NMR (400 MHz, CDCl3) δ 6.75 (s, 2H), 3.91 (m, 6H), 3.79 (t, J=5.8 Hz, 2H), 3.71-3.56 (m, 14H), 3.39 (t, J=5.6 Hz, 2H), 3.39 (brs, 4H), 2.97 (t, J=6.1 Hz, 2H), 2.67 (br s, 2H). LCMS: 652.5 [M+H]

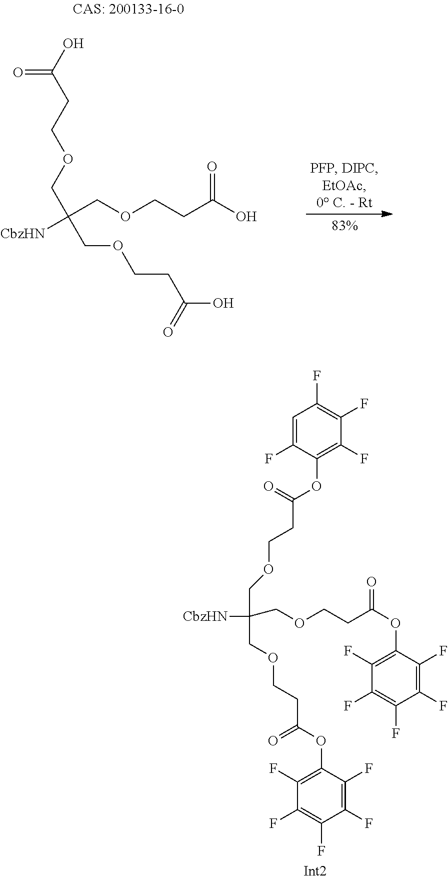 [SMILES: O=C(O)CCOCC(COCCC(=O)O)(COCCC(=O)O)NC(=O)OCc1ccccc1 ; O=C(CCOCC(COCCC(=O)Oc1c(F)c(F)c(F)c(F)c1F)(COCCC(=O)Oc1c(F)c(F)c(F)c(F)c1F)NC(=O)OCc1ccccc1)Oc1c(F)cc(F)c(F)c1F]

### Synthesis of bis(perfluorophenyl) 3,3′-((2-(((benzyloxy)carbonyl)amino)-2-((3-oxo-3-(perfluorophenoxy)propoxy)methyl)propane-1,3-diyl)bis(oxy))dipropionate (Int2)

A solution of 3,3′-((2-(((benzyloxy)carbonyl)amino)-2-((2-carboxyethoxy)methyl)propane-1,3-diyl)bis(oxy))dipropionic acid (1.0 eq, 10.0 g, 21.2 mmol) in ethyl acetate (100 mL) was cooled at 0° C., 2,3,4,5,6-pentafluorophenol (3.0 eq, 11.7 g, 63.6 mmol) and N,N′-diisopropylcarbodiimide (4.0 eq, 13.3 mL, 84.8 mmol) were added and reaction mixture was stirred at room temperature for 16 h. After completion, reaction mixture was filtered through sintered funnel (without celite) and filtrate was concentrated to a crude residue, which was purified by column chromatography using silica gel (100-200 mesh) and 0-15% ethyl acetate in hexane to afford bis(perfluorophenyl) 3,3′-((2-(((benzyloxy)carbonyl)amino)-2-((3-oxo-3-(perfluorophenoxy)propoxy)methyl)propane-1,3-diyl)bis(oxy))dipropionate (Int2) as an off white solid. Yield: 17.0 g, 82.66%; LCMS m/z 970.39 [M+H]; ¹H NMR (400 MHz, DMSO-d₆) δ 7.34-7.26 (m, 5H), 6.58 (s, 1H), 4.95 (s, 2H), 3.75 (t, J=5.6 Hz, 6H), 3.59 (s, 6H), 2.98 (t, J=5.6 Hz, 6H).

### Synthesis of benzyl (1,31-bis((2R,3R,4R,5R,6R)-3-acetamido-4,5-dihydroxy-6-(hydroxymethyl)tetrahydro-2H-pyran-2-yl)-16-(15-((2R,3R,4R,5R,6R)-3-acetamido-4,5-dihydroxy-6-(hydroxymethyl)tetrahydro-2H-pyran-2-yl)-5-oxo-2,9,12-trioxa-6-azapentadecyl)-11,21-dioxo-4,7,14,18,25,28-hexaoxa-10,22-diazahentriacontan-16-yl)carbamate

A solution of N-[(2R,3R,4R,5R,6R)-2-[3-[2-(2-aminoethoxy)ethoxy]propyl]-4,5-dihydroxy-6-(hydroxymethyl)tetrahydropyran-3-yl]acetamide (Compound 1248, 3.30 eq, 346 mg, 0.987 mmol) and (2,3,4,5,6-pentafluorophenyl) 3-[2-(benzyloxycarbonylamino)-3-[3-oxo-3-(2,3,4,5,6-pentafluorophenoxy)propoxy]-2-[[3-oxo-3-(2,3,4,5,6-pentafluorophenoxy)propoxy]methyl]propoxy]propanoate (1.00 eq, 290 mg, 0.299 mmol) in DMSO (1.4955 mL) was treated with diisopropylethylamine (DIPEA) (3.00 eq, 156 uL, 0.897 mmol). After 90 minutes, the reaction was diluted with water (500 uL), formic acid (150 uL) and methanol (350 uL) and the reaction was purified by RPHPLC (3-50% ACN in water w/0.1% FA) to give benzyl N—[ 2-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-1,1-bis[[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]methyl]ethyl]carbamate as a white solid. Yield: 405 mg, 92%. LCMS m/z 1469.1 [M+H]

### Synthesis of N-[2-[2-[3-[(2R,3R,4R,5R,6R)-3-acetamido-4,5-dihydroxy-6-(hydroxymethyl)tetrahydropyran-2-yl]propoxy]ethoxy]ethyl]-3-[3-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-2-[[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]methyl]-2-amino-propoxy]propenamide

A mixture of benzyl N-[2-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-1,1-bis[[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]methyl]ethyl]carbamate (1.00 eq, 390 mg, 0.266 mmol) and 10% Pd/C, Evonik Noblyst (0.700 eq, 396 mg, 0.186 mmol) in methanol (5.3 mL) was stirred vigorously under an atmosphere of nitrogen gas. After 1 h, the reaction was filtered through celite, the filter cake was washed with methanol (50 mL) and the filtrate was concentrated under reduced pressure to a syrup. The syrup (~1.8 mL) was filtered (some darkness of carbon observed) then dripped into EtOAc (8 mL) but a sticky ppt was formed so the slurry was concentrated under reduced pressure to give N-[2-[2-[3-[(2R,3R,4R,5R,6R)-3-acetamido-4,5-dihydroxy-6-(hydroxymethyl)tetrahydropyran-2-yl]propoxy]ethoxy]ethyl]-3-[3-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-2-[[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]methyl]-2-amino-propoxy]propenamide as a white solid. The crude material was used in the next step without further purification. Yield: 326 mg, 92%. LCMS m/z 1335.1 [M+H]

### Synthesis of 4-(33-((2R,3R,4R,5R,6R)-3-acetamido-4,5-dihydroxy-6-(hydroxymethyl)tetrahydro-2H-pyran-2-yl)-18,18-bis(15-((2R,3R,4R,5R,6R)-3-acetamido-4,5-dihydroxy-6-(hydroxymethyl)tetrahydro-2H-pyran-2-yl)-5-oxo-2,9,12-trioxa-6-azapentadecyl)-16,23-dioxo-4,7,10,13,20,27,30-heptaoxa-17,24-diazatritriacontanoyl)-1-(2-(2,5-dioxo-2,5-dihydro-1H-pyrrol-1-yl)ethyl)piperazin-1-ium (Compound 1248)

A mixture of N-[2-[2-[3-[(2R,3R,4R,5R,6R)-3-acetamido-4,5-dihydroxy-6-(hydroxymethyl)tetrahydropyran-2-yl]propoxy]ethoxy]ethyl]-3-[3-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-2-[[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]methyl]-2 -amino-propoxy]propanamide (1.00 eq, 250 mg, 0.187 mmol) and (2,3,4,5,6-pentafluorophenyl) 3-[2-[2-[2-[3-[4-[2-(2,5-dioxopyrrol-1-yl)ethyl]piperazin-1-yl]-3-oxo-propoxy]ethoxy]ethoxy]ethoxy]propanoate; 2,2,2-trifluoroacetic acid (1.20 eq, 172 mg, 0.225 mmol) in DMF (1.8734 mL) was treated with diisopropylethylamine (4.00 eq, 131 μL, 0.749 mmol) and the reaction was stirred at room temperature. After 19h, another charge of PFP-ester was added (0.24 eq, 34 mg). After 50h, another charge of PFP-ester (0.26 eq, 40 mg) was added. After 68 h, the reaction was diluted with DMSO (500 uL) then purified by RPHLC (2% then 10% then 10-20% ACN in water w/0.1% TFA) to give Compound 1248 as a white solid. Yield: 245 mg, 68%. LCMS m/z 902.3 [M+2H]++. HPLC: 99% based on ELSD.

### Synthesis of Compound 1255: General Method B

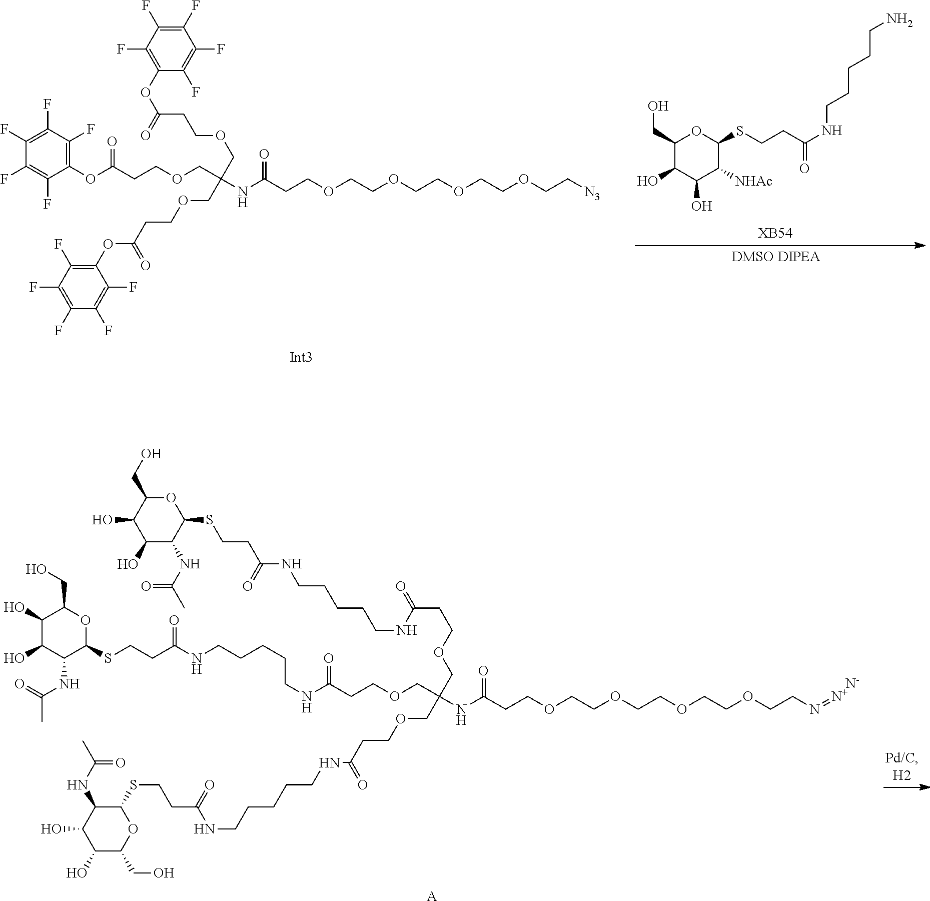

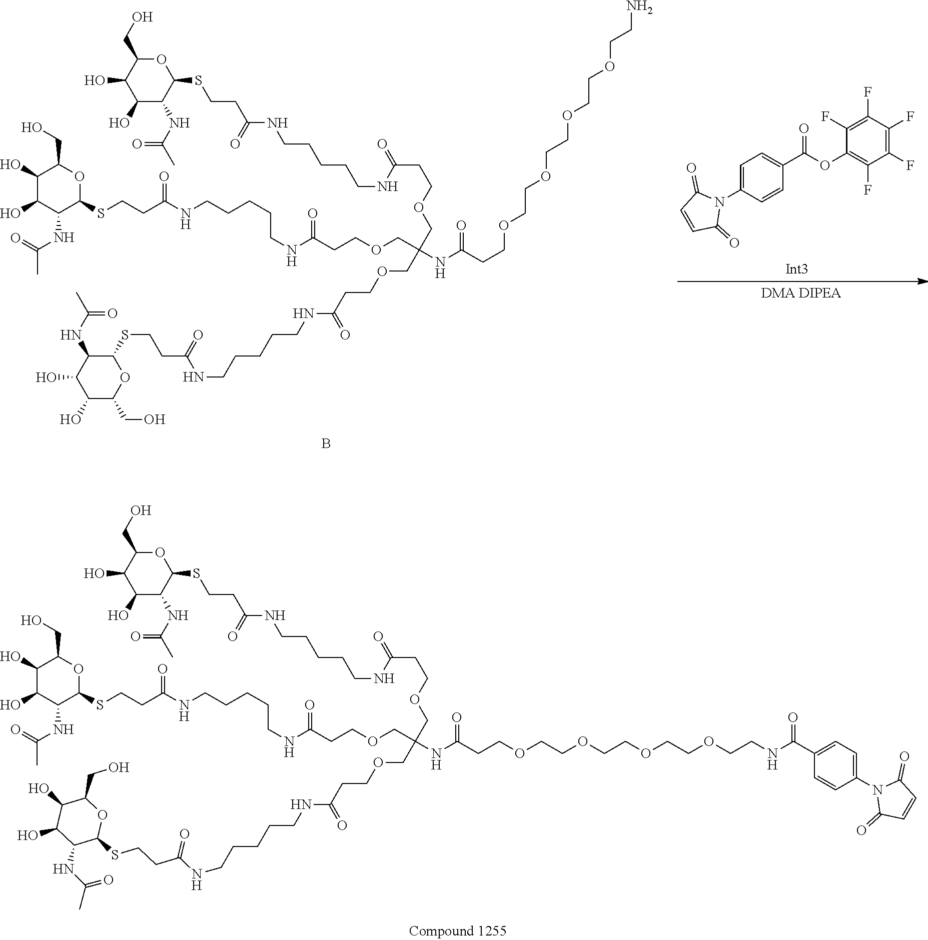

### Synthesis of (Cmpd A)

A solution of perfluorophenyl 1-azido-15-oxo-17,17-bis((3-oxo-3-(perfluorophenoxy)propoxy)methyl)-3,6,9,12,19-pentaoxa-16-azadocosan-22-oate (Int3, 1.0 eq, 72.0 mg, 0.0649 mmol) and DIPEA (9.0 eq, 102 mL, 0.584 mmol) in DMSO (649 mL) was treated with 3-[(2S,3R,4R,5R,6R)-3-acetamido-4,5-dihydroxy-6-(hydroxymethyl)tetrahydropyran-2-yl]sulfanyl-N-(5-aminopentyl)propanamide (XB54, 3.3 eq, 84.3 mg, 0.214 mmol). After 2 h, the reaction was purified by reversed-phase HPLC (10-50% acetonitrile in water w/0.1% TFA) to give (Cmpd A)N-[2-[3-[5-[3-[(2S,3R,4R,5R,6R)-3-acetamido-4,5-dihydroxy-6-(hydroxymethyl)tetrahydropyran-2-yl]sulfanylpropanoylamino]pentylamino]-3-oxo-propoxy]-1,1-bis[[3-[5-[3-[(2S,3R,4R,5R,6R)-3-acetamido-4,5-dihydroxy-6-(hydroxymethyl)tetrahydropyran-2-yl]sulfanylpropanoylamino]pentylamino]-3-oxo-propoxy]methyl]ethyl]-3-[2-[2-[2-(2-azidoethoxy)ethoxy]ethoxy]ethoxy]propanamide. Yield: 81 mg, 72%. LCMS m/z 1737.4 [M+H].

### Synthesis of (Cmpd B)

A mixture of CmpdA (1.0 eq, 42.0 mg, 0.0242 mmol), 10% Pd/C, Evonik Noblyst (0.84 eq, 43.4 mg, 0.0204 mmol) and acetic acid (1.67 mL) was placed under an hydrogen atmosphere via balloon for 1 h. The reaction was filtered through celite then a 0.2 mm syringe filter then concentrated under reduced pressure. The residue was concentrated from water then from MeCN before leaving under high vacuum to give Cmpd B. Yield: 40 mg, 93%. LCMS m/z 1710.8 [M+H].

### Synthesis of (Compound 1255)

A cold solution of perfluorophenyl 4-(2,5-dioxo-2,5-dihydro-1H-pyrrol-1-yl)benzoate (1.2 eq, 5.2 mg, 0.0136 mmol) in DMA (100 mL) was added to a cold solution of Cmpd B (1.0 eq, 20.0 mg, 0.0113 mmol) and DIPEA (4.00 eq, 7.9 mL, 0.0452 mmol) in DMA (100 mL) being cooled in an ice bath. After 90 minutes, the reaction was diluted with DMSO, water and some methanol for rinsing then the crude solution was purified by reversed-phase HPLC (5-50% MeCN in water w/0.1% formic acid) to give Compound 1255. Yield: 10 mg, 48%. LCMS m/z 1910.6 [M+H].

### Synthesis of Int3

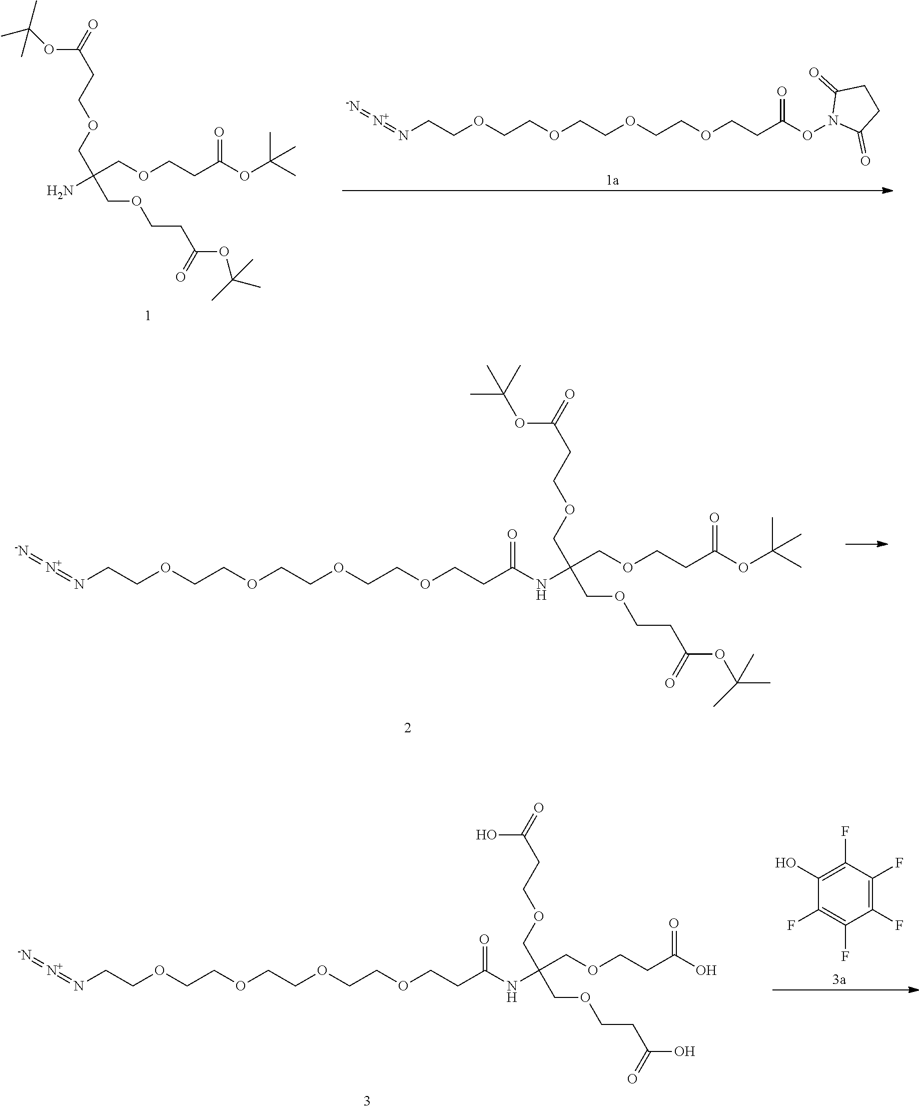

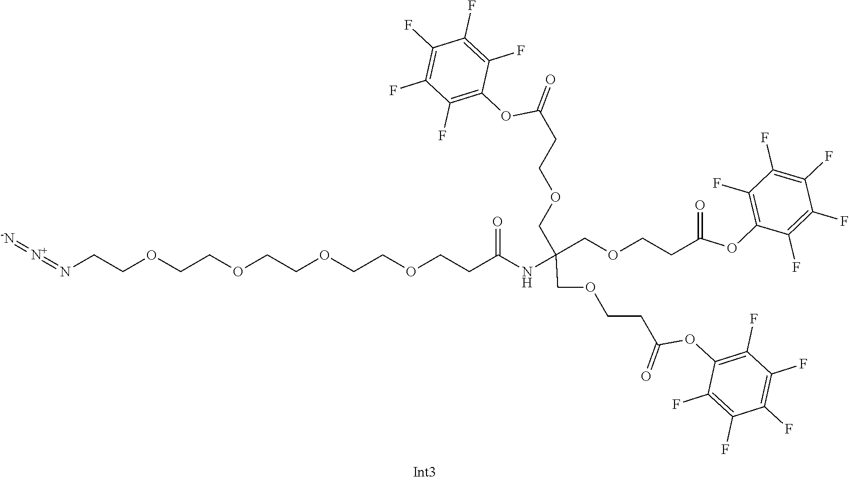

### Synthesis of tert-butyl 1-azido-17,17-bis((3-(tert-butoxy)-3-oxopropoxy)methyl)-15-oxo-3,6,9,12,19-pentaoxa-16-azadocosan-22-oate (2)

A solution of di-tert-butyl 3,3′-((2-amino-2-((3-(tert-butoxy)-3-oxopropoxy)methyl)propane-1,3-diyl)bis(oxy))dipropionate (1, CAS: 175724-30-8) (452 mg, 0.894 mmol, 1.00 eq.) and 2,5-dioxopyrrolidin-1-yl 1-azido-3,6,9,12-tetraoxapentadecan-15-oate (1a) (356 mg, 0.916 mmol, 1.02 eq.) in 1 mL acetonitrile was stirred under nitrogen atmosphere and heated at 45 C for 2 days until minimal Compound 1 remained. The reaction mixture was diluted with dichloromethane and evaporated onto silica, then purified by flash column chromatography column, eluting with 0-100% ethyl acetate/dichloromethane. Fractions containing product were concentrate and dried further under high vacuum at ambient temperature to afford Compound 2 as a clear thick syrup. Yield: 559 mg (78%); LCMS m/z 779.2 [M+H].

### Synthesis of 1-azido-17,17-bis((2-carboxyethoxy)methyl)-15-oxo-3,6,9,12,19-pentaoxa-16-azadocosan-22-oic acid (3)

To tert-butyl 1-azido-17,17-bis((3-(tert-butoxy)-3-oxopropoxy)methyl)-15-oxo-3,6,9,12,19-pentaoxa-16-azadocosan-22-oate (2) (509 mg, 0.654 mmol) was added 10 mL of a pre-mixed solution of 30% trifluoroacetic acid in dichloromethane. The mixture stirred at ambient temperature for 3.5 hrs., until consumption of Compound 2. The solvent was evaporated to residue under a stream of nitrogen, dried further under high vacuum at ambient temperature, then dissolved in 20% water/acetonitrile and lyophilized to dryness to afford Compound 3 as a thick dark yellow color syrup. Yield: 424 mg (99%); LCMS m/z 611.3 [M+H], 609.4 [M−1]⁻.

### perfluorophenyl 1-azido-15-oxo-17,17-bis((3-oxo-3-(perfluorophenoxy)propoxy)methyl)-3,6,9,12,19-pentaoxa-16-azadocosan-22-oate (Int3)

A solution of 1-azido-17,17-bis((2-carboxyethoxy)methyl)-15-oxo-3,6,9,12,19-pentaoxa-16-azadocosan-22-oic acid (3) (81.3 mg, 0.133 mmol, 1.0 eq.) and 2,3,4,5,6-pentafluorophenol (3a) (91.8 mg, 0.499 mmol, 3.7 eq.) in 1.5 mL of dichloromethane was added N,N′-diisopropylcarbodiimide (90 mL, 0.581 mmol, 4.4 eq.). The mixture stirred at ambient temperature for 1 hr, until consumption of Compound 3. The reaction mixture was diluted with dichloromethane, evaporated onto silica, then purified by flash column chromatography column, eluting with 0-100% ethyl acetate/hexanes. Fractions containing product were concentrated and dried further under high vacuum at ambient temperature to afford Int3 as a clear thick syrup. Yield: 104 mg (67%); LCMS m/z 1109.1 [M+H].

### Synthesis of Compound 1251: General Synthesis, Method C

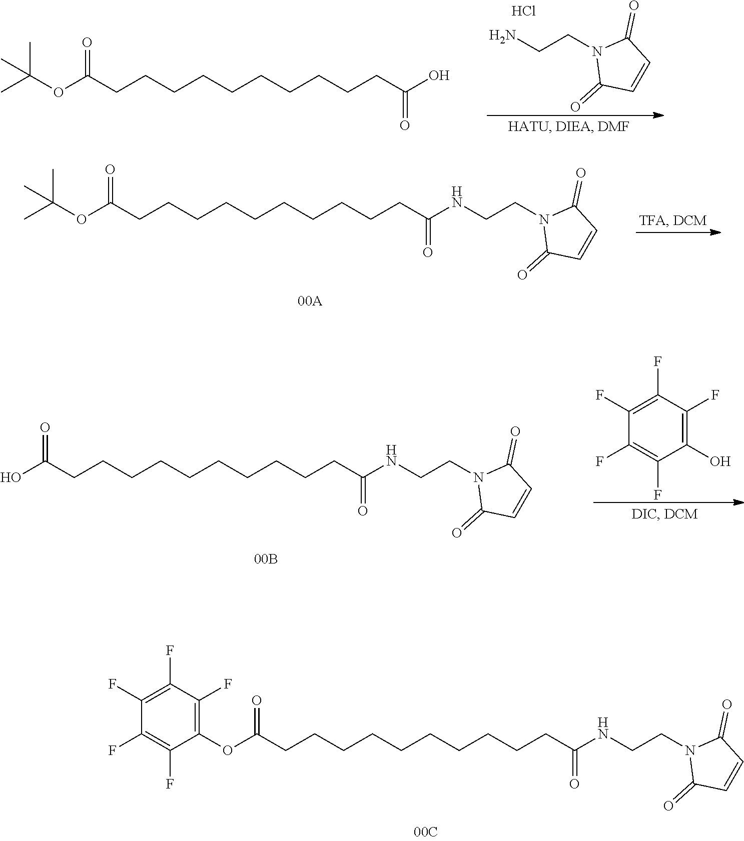

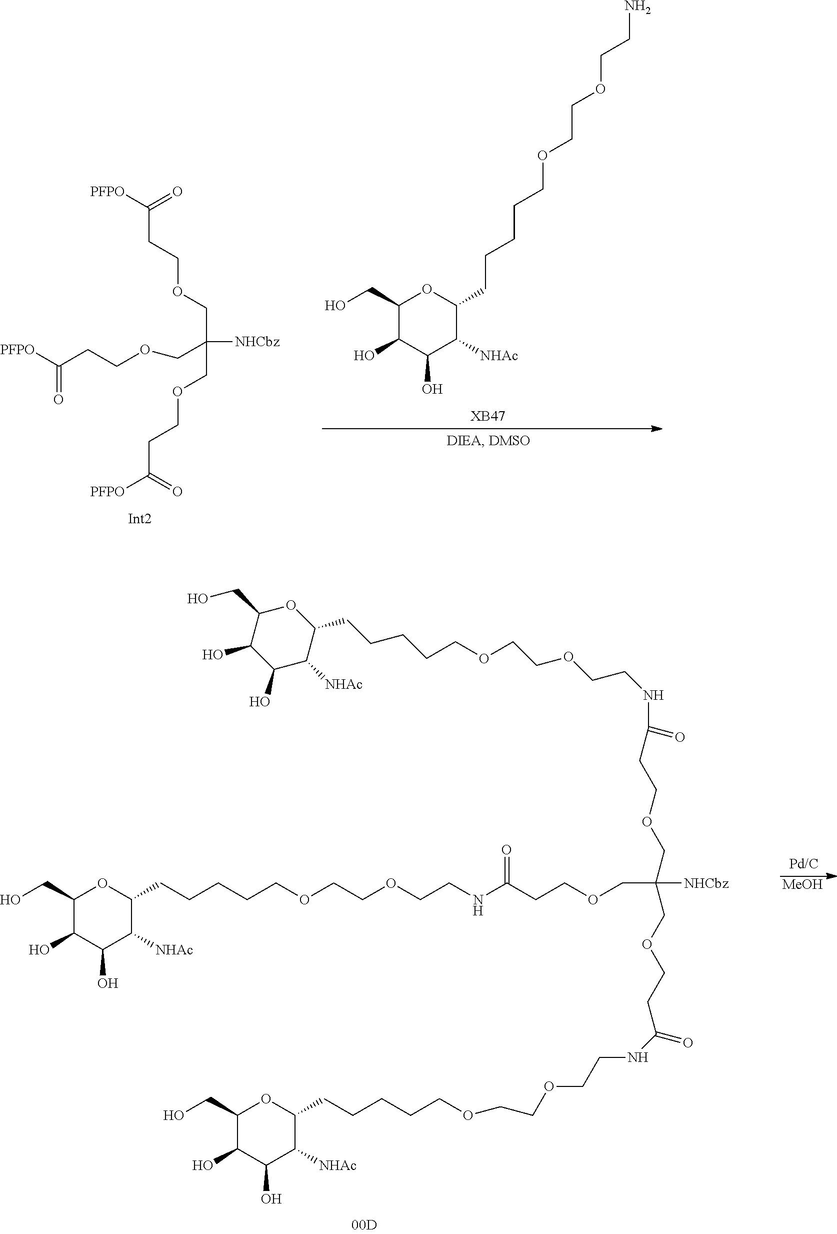

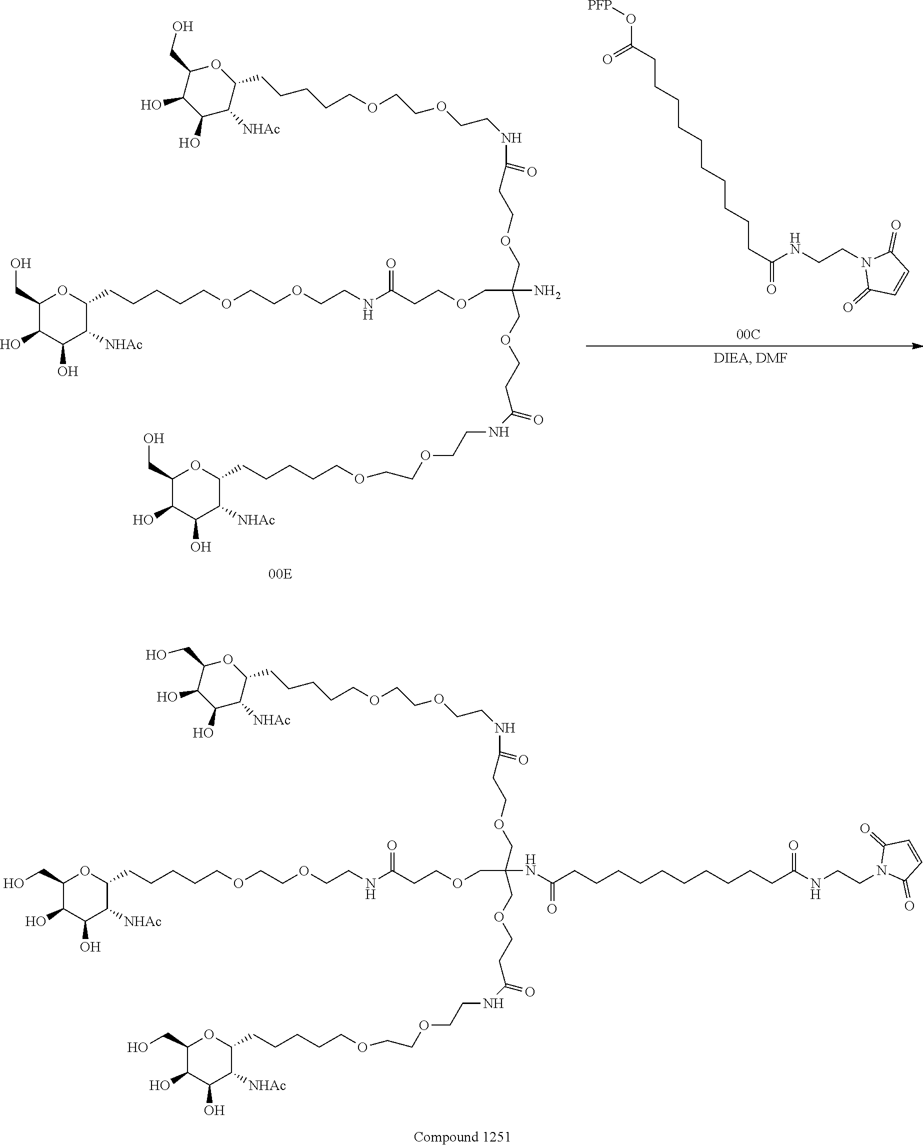

To a mixture of 12-tert-butoxy-12-oxo-dodecanoic acid (1.00 eq, 553 mg, 1.93 mmol) in DMF (9.4 mL) were added Diisopropylethylamine (DIPEA) (4.00 eq, 1.3 mL, 7.72 mmol) and HATU (1.20 eq, 881 mg, 2.32 mmol), followed by addition of 1-(2-aminoethyl)pyrrole-2,5-dione; hydrochloride (1.00 eq, 341 mg, 1.93 mmol). The mixture was stirred at room temperature for 1h, diluted with EtOAc, washed with water (2×) and brine (1×), dried, concentrated, and purified by column (0-90% EtOAc/hexane) to give 00A as a white solid (644 mg, yield: 82%). LCMS m/z 431.2 [M+Na]⁺.

To a mixture of tert-butyl 12-[2-(2,5-dioxopyrrol-1-yl)ethylamino]-12-oxo-dodecanoate (00A, 1.00 eq, 581 mg, 1.42 mmol) in DCM (2 mL) was added TFA (6 mL). The mixture was stirred at room temperature for 2h, concentrated, and lyophilized to give 00B as a white solid (502 mg, yield: 100%). LCMS m/z 353.2 [M+H]

To a mixture of 12-[2-(2,5-dioxopyrrol-1-yl)ethylamino]-12-oxo-dodecanoic acid (00B, 1.00 eq, 502 mg, 1.42 mmol) and 2,3,4,5,6-Pentafluorophenol (1.20 eq, 315 mg, 1.71 mmol) in DCM (6 mL) was added 1,3-diisopropylcarbodiimide (1.50 eq, 0.33 mL, 2.14 mmol). The mixture was stirred at room temperature for 2h, filtered, concentrated, purified by column (0-90% EtOAc/hexane) to give 00C as a white solid (728.5 mg, yield: 99%). LCMS m/z 519.1 [M+H].

To a mixture of (2,3,4,5,6-pentafluorophenyl) 3-[2-(benzyloxycarbonylamino)-3-[3-oxo-3-(2,3,4,5,6-pentafluorophenoxy)propoxy]-2-[[3-oxo-3-(2,3,4,5,6-pentafluorophenoxy)propoxy]methyl]propoxy]propanoate (Int2, 1.00 eq, 29.0 mg, 0.0299 mmol) and N-[(2R,3R,4R,5R,6R)-2-[5-[2-(2-aminoethoxy)ethoxy]pentyl]-4,5-dihydroxy-6-(hydroxymethyl)tetrahydropyran-3-yl]acetamide; 2,2,2-trifluoroacetic acid (XB47, 3.10 eq, 45.7 mg, 0.0927 mmol) in DMSO (0.7 mL) was added DIPEA (10.0 eq, 0.052 mL, 0.299 mmol). The mixture was stirred at room temperature for 1 h and was purified by prep. HPLC (2-40% MeCN/water with 0.1% TFA) to give OOD as a white solid (42.2 mg, yield: 91%). LCMS m/z 1552.6 [M+H].

To a mixture of OOD (1.00 eq, 42.2 mg, 0.0272 mmol) in MeOH (4 mL) was added 10% Pd/C (14 mg). The mixture was stirred at room temperature under hydrogen for 1 h, filtered, concentrated to give 00E as a white solid. (38.8 mg, yield: 100%) LCMS m/z 1418.8 [M+H]

To a mixture of (2,3,4,5,6-pentafluorophenyl) 12-[2-(2,5-dioxopyrrol-1-yl)ethylamino]-12-oxo-dodecanoate (00C, 1.50 eq, 10.1 mg, 0.0196 mmol) in DMF (0.7 mL) were added N-[2-[2-[15- [(2R,3R,4R,5R,6R)-3-acetamido-4,5-dihydroxy-6-(hydroxymethyl)tetrahydropyran-2-yl]pentoxy]ethoxy]ethyl]-3-[3-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylamino]-3-oxo-propoxy]-2-[[3-[2-[2-[5-[(2R,3R,4R,5R,6R)-3-acetamido-4,5-dihydroxy-6-(hydroxymethyl)tetrahydropyran-2-yl]pentoxy]ethoxy]ethylamino]-3-oxo-propoxy]methyl]-2-amino-propoxy]propanamide (00E, 1.00 eq, 18.5 mg, 0.0130 mmol) and Diisopropylethylamine (DIPEA) (4.00 eq, 0.0091 mL, 0.0522 mmol). The mixture was stirred at room temperature for 5 h and more DIPEA (2.5 mL) was added. The mixture was stirred at room temperature overnight. More DIEA (2 mL) was added, and the mixture was stirred at room temperature for 5 h and purified by prep. HPLC (2-50% MeCN/water with 0.1% TFA) to give Compound 1251 as a white solid (12.8 mg, yield: 56%). LCMS m/z 1753.7 [M+H].

### Synthesis of Compound 2378: General Synthesis Method F

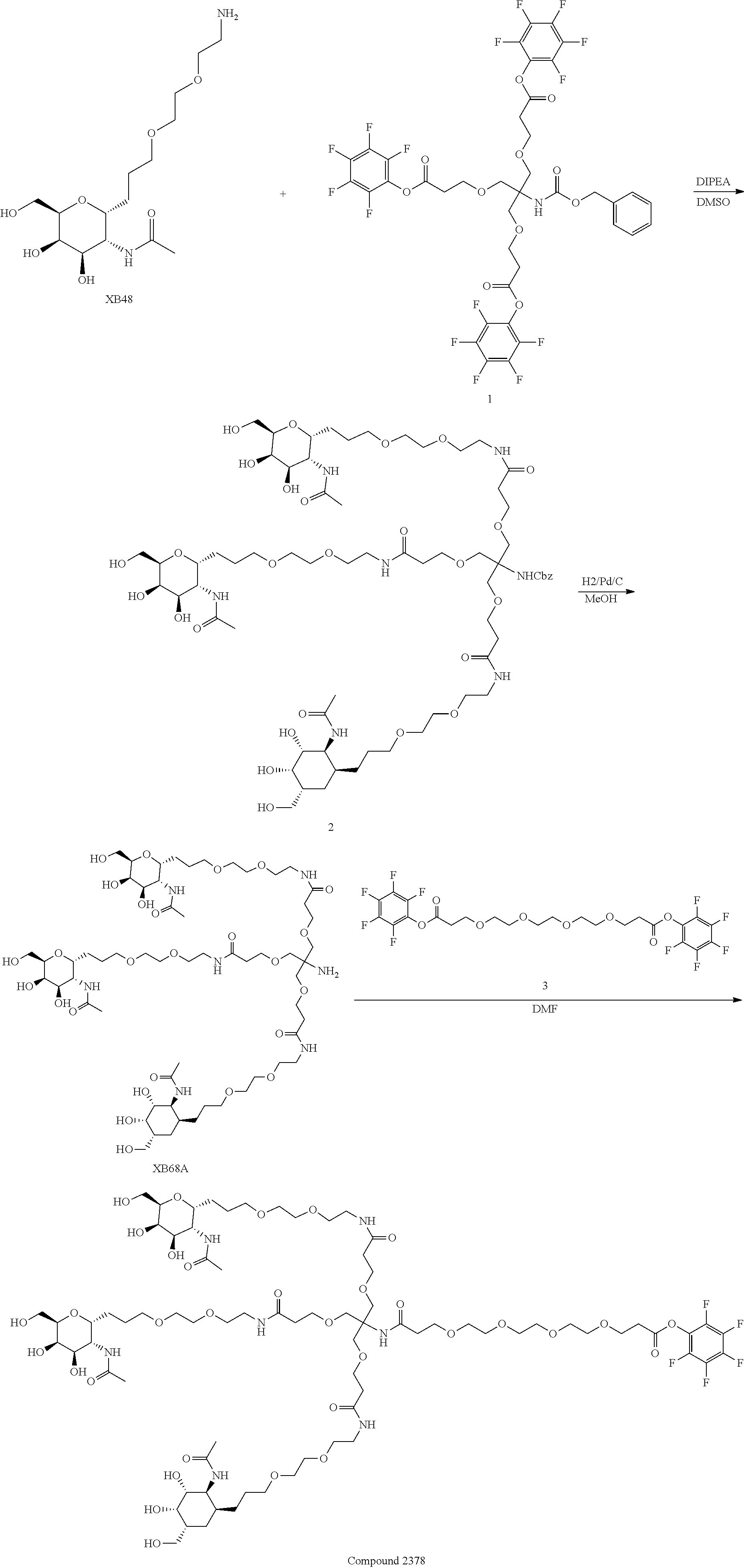

### Synthesis of benzyl (1,31-bis((2R,3R,4R,5R,6R)-3-acetamido-4,5-dihydroxy-6-(hydroxymethyl)tetrahydro-2H-pyran-2-yl)-16-(15-((2R,3R,4R,5R,6R)-3-acetamido-4,5-dihydroxy-6-(hydroxymethyl)tetrahydro-2H-pyran-2-yl)-5-oxo-2,9,12-trioxa-6-azapentadecyl)-11,21-dioxo-4,7,14,18,25,28-hexaoxa-10,22-diazahentriacontan-16-yl)carbamate (2)

A mixture of N-((2R,3R,4R,5R,6R)-2-(3-(2-(2-aminoethoxy)ethoxy)propyl)-4,5-dihydroxy-6-(hydroxymethyl)tetrahydro-2H-pyran-3-yl)acetamide (XB48, 3.24 eq, 1.72 g, 4.91 mmol) and bis(perfluorophenyl) 3,3′-((2-(((benzyloxy)carbonyl)amino)-2-((3-oxo-3-(perfluorophenoxy)propoxy)methyl)propane-1,3-diyl)bis(oxy))dipropionate (1.00 eq, 1.47 g, 1.52 mmol) was treated with diisopropylethylamine (1, 3.00 eq, 862 mL, 4.95 mmol) then dissolved in DMSO (6.89 mL) with sonication. After 90 minutes the reaction was purified by reversed-phase HPLC (5% then 15-50% acetonitrile in water w/0.1% FA) to give 2. Yield: 2.21 g, 91%. LCMS 1468.6 [M+H].

### Synthesis of N-[2-[2-[3-[(2R,3R,4R,5R,6R)-3-acetamido-4,5-dihydroxy-6-(hydroxymethyl)tetrahydropyran-2-yl]propoxy]ethoxy]ethyl]-3-[3-[3-[2-[2-[3-[(2R,3R,4R,5R,6R)-3-acetamido-4,5-dihydroxy-6-(hydroxymethyl)tetrahydropyran-2-yl]propoxy]ethoxy]ethylamino]-3-oxo-propoxy]-2-[[3-[2-[2-[3-[(2R,3R,4R,5R,6R)-3-acetamido-4,5-dihydroxy-6 -(hydroxymethyl)tetrahydropyran-2-yl]propoxy]ethoxy]ethylamino]-3-oxo-propoxy]methyl]-2-amino-propoxy]propenamide (XB68A)

A solution of 2 (1.00 eq, 400 mg, 0.272 mmol) in methanol (20 mL) was purged with nitrogen then treated with 10% Pd/C (0.400 eq, 232 mg, 0.109 mmol) before being evacuated then back-filled with hydrogen via balloon. After 2 h, the reaction was treated with 500 mg celite then filtered over a pad of celite. The filter cake was rinsed with methanol and the filtrate was concentrated under reduced pressure. The residue was dissolved in water with minimal DMSO then purified by reversed-phase HPLC (5-50% acetonitrile in water w/0.2 mM NH₄OH) to give XB68A. Yield: 315 mg, 86.7%. LCMS 1335.6 [M+H].

### Synthesis of perfluorophenyl 33-((2R,3R,4R,5R,6R)-3-acetamido-4,5-dihydroxy-6-(hydroxymethyl)tetrahydro-2H-pyran-2-yl)-18,18-bis(15-((2R,3R,4R,5R,6R)-3-acetamido-4,5-dihydroxy-6-(hydroxymethyl)tetrahydro-2H-pyran-2-yl)-5-oxo-2,9,12-trioxa-6-azapentadecyl)-16,23-dioxo-4,7,10,13,20,27,30-heptaoxa-17,24-diazatritriacontanoate (Compound 2378)

A solution of XB68A (1.00 eq, 1.10 g, 0.824 mmol) in DMF (13.7 mL) was added to a solution of bis(perfluorophenyl) 4,7,10,13-tetraoxahexadecanedioate (3, 3.00 eq, 1.55 g, 2.47 mmol) in DMF (5.6 mL) drop-wise via syringe over 16 minutes. Next, DMF (2 mL) was added to the amine flask for rinsing then this solution was added to the reaction dropwise via syringe. The reaction was stirred at room temperature overnight. After 26 h, the reaction was purified directly by reversed-phase HPLC (5-15-40% acetonitrile in water w/0.1% TFA) to give solids that were not easily manipulated. The residue was treated with acetonitrile to remove water via azeotropic distillation. The residue was then dissolved in MeOH (1 volume) then diluted with DCM (95 volumes) then concentrated under high vacuum for 18 h to give Compound 2378 as a nice powder. Yield: 1050 mg, 71%. LCMS 1777.58 [M+H].

### Synthesis of Compound 1117 (I-168)

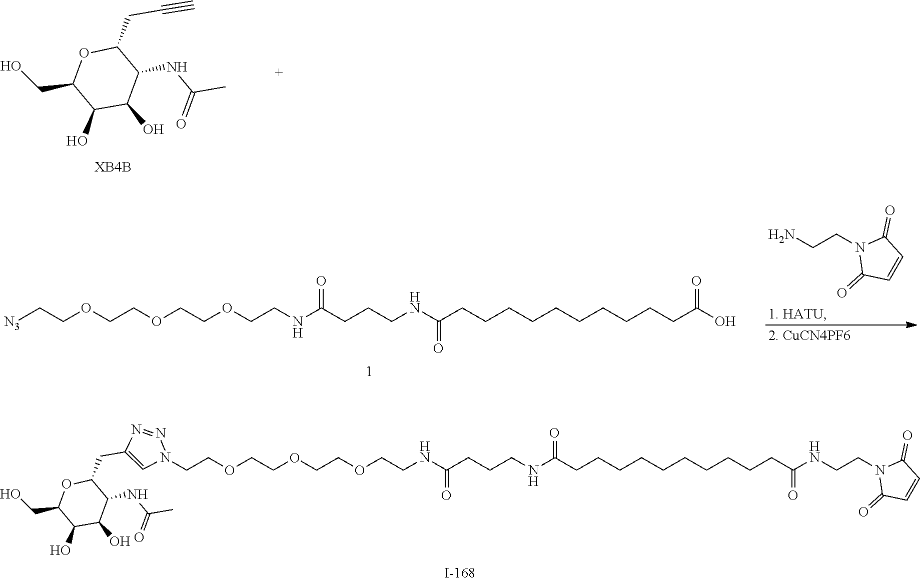 [SMILES: C#CC[C@H]1O[C@H](CO)[C@H](O)[C@H](O)[C@H]1NC(C)=O ; [N-]=[N+]=NCCOCCOCCOCCNC(=O)CCCNC(=O)CCCCCCCCCCC(=O)O ; NCCN1C(=O)C=CC1=O ; CC(=O)N[C@@H]1[C@@H](O)[C@@H](O)[C@@H](CO)O[C@@H]1Cc1cn(CCOCCOCCOCCNC(=O)CCCNC(=O)CCCCCCCCCCC(=O)NCCN2C(=O)C=CC2=O)nn1]

To a mixture of compound 1-azido-13,18-dioxo-3,6,9-trioxa-12,17-diazanonacosan-29-oic acid (1, 1.00 eq, 14.7 mg, 0.0285 mmol) in DMSO (0.5 mL) were added DIEA (4.00 eq, 0.020 mL, 0.114 mmol) and HATU (1.10 eq, 11.9 mg, 0.0314 mmol), followed by addition of 2-maleimidoethylamine hydrochloride (1.00 eq, 5.0 mg, 0.0285 mmol). The mixture was stirred at room temperature for 30 minutes and to this mixture was added compound XB4B (1.00 eq, 6.9 mg, 0.0285 mmol). The mixture was purged with nitrogen and tetrakis(acetonitrile)copper(I) hexafluorophosphate (2.00 eq, 21.5 mg, 0.0570 mmol) was added. The mixture was stirred at room temperature for 3 h. The mixture was purified by prep. HPLC (10-40% MeCN/water with 0.1% TFA) to give N1-(1-(4-(((2R,3R,4R,5R,6R)-3-acetamido-4,5-dihydroxy-6-(hydroxymethyl)tetrahydro-2H-pyran-2-yl)methyl)-1H-1,2,3-triazol-1-yl)-13-oxo-3,6,9-trioxa-12-azahexadecan-16-yl)-N12-(2-(2,5-dioxo-2,5-dihydro-1H-pyrrol-1-yl)ethyl)dodecanediamide (Compound 1117, 1-168) as a white solid. Yield: 11.8 mg, 47%. LCMS m/z 881.2 [M+H]⁺.

### Synthesis of Compound 2403

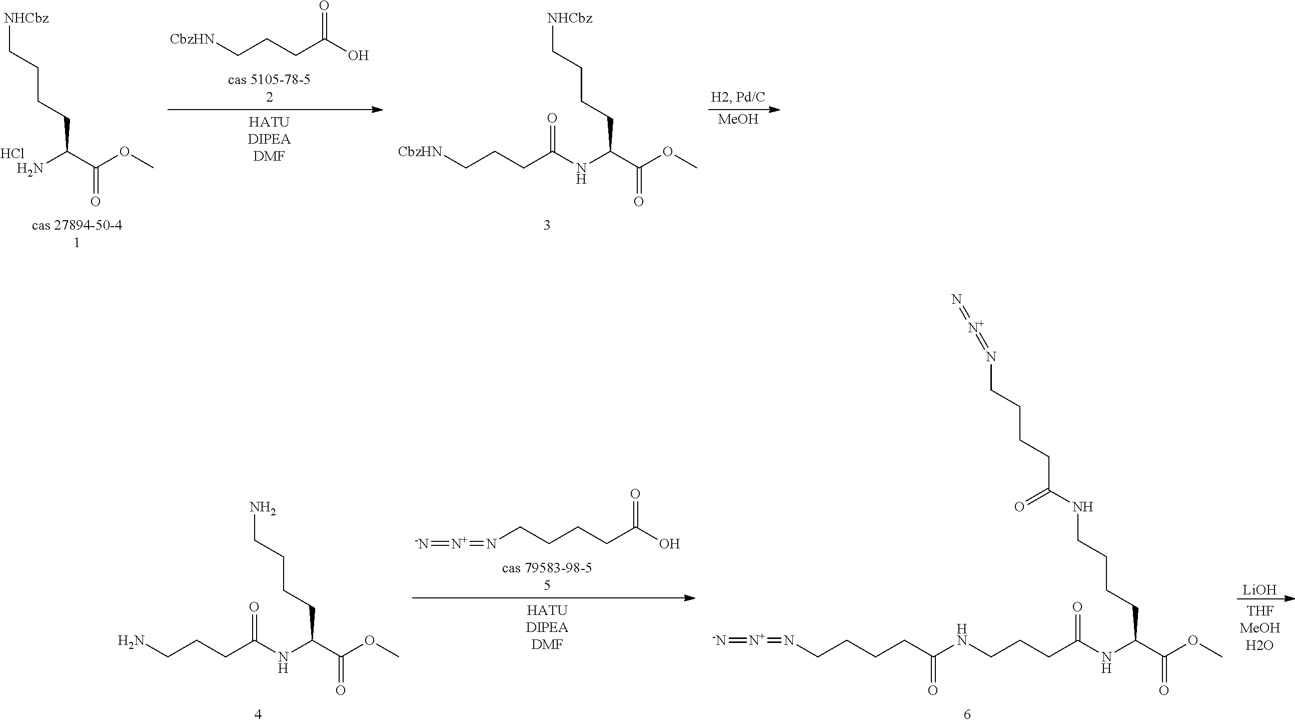

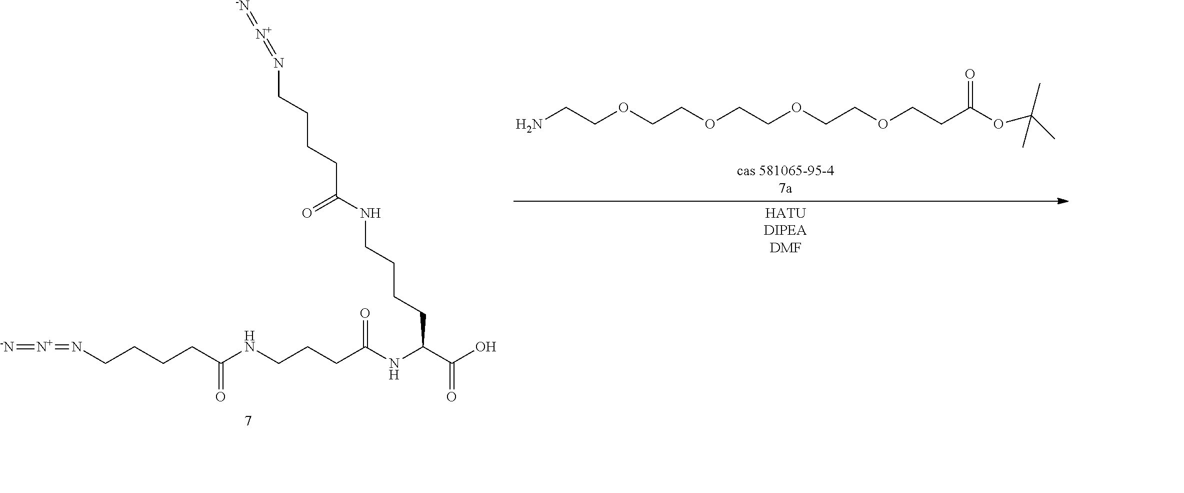

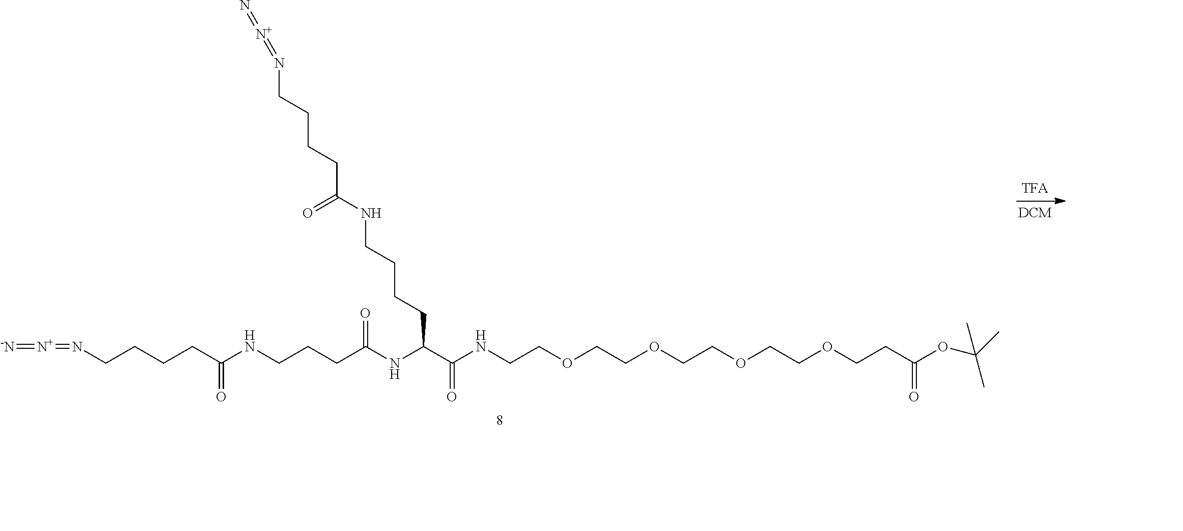

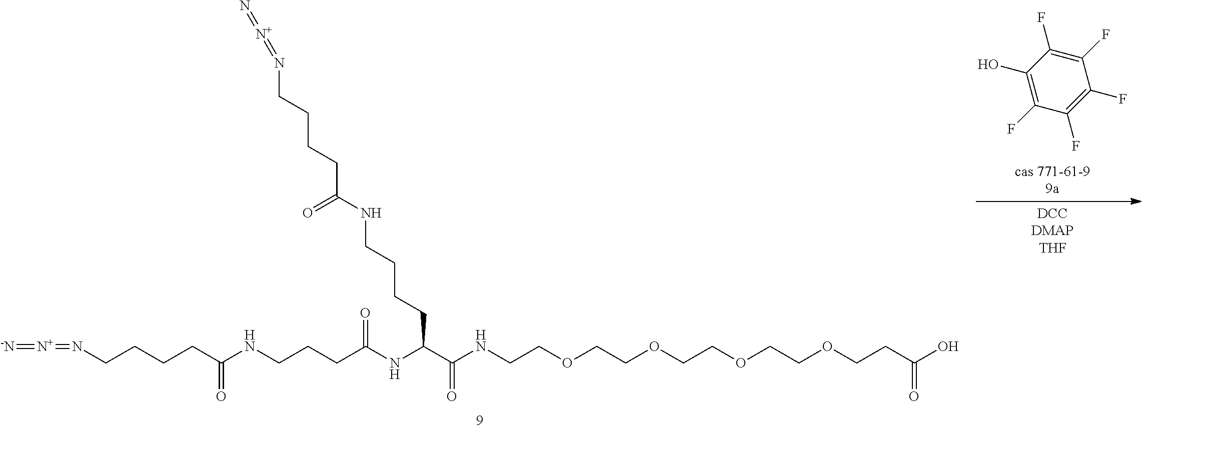

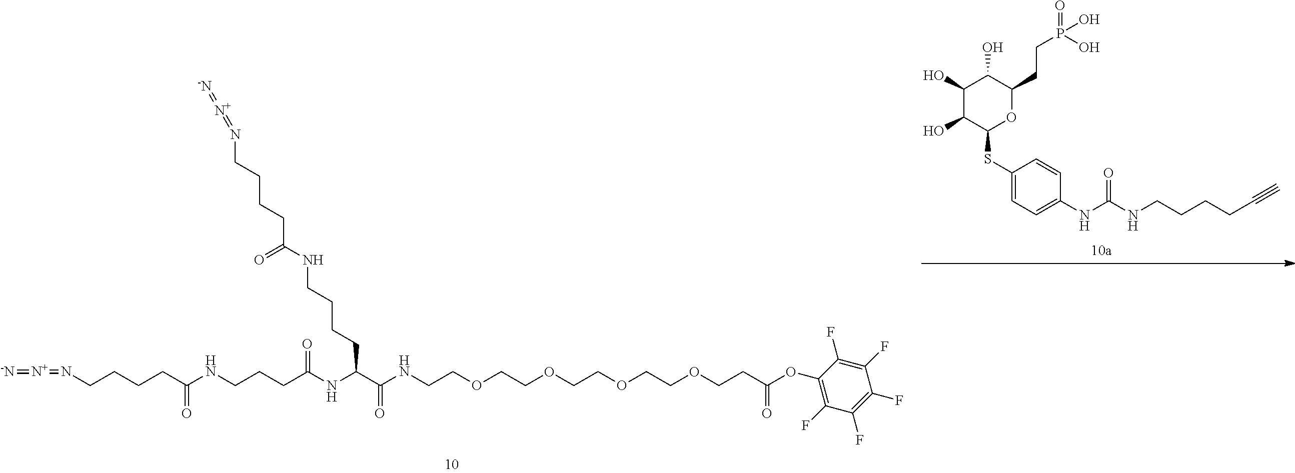

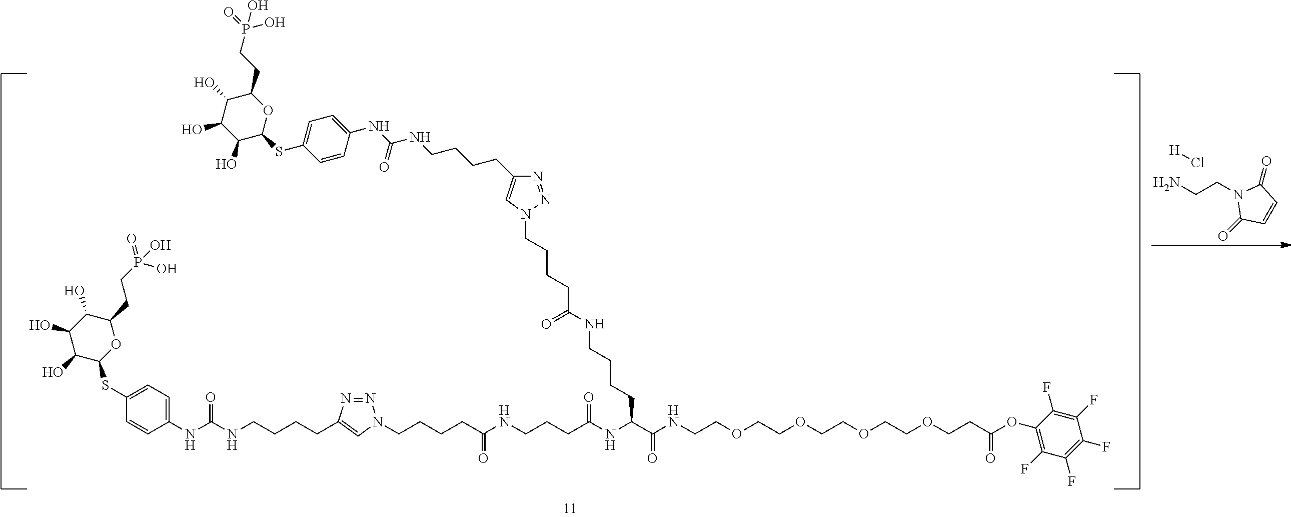

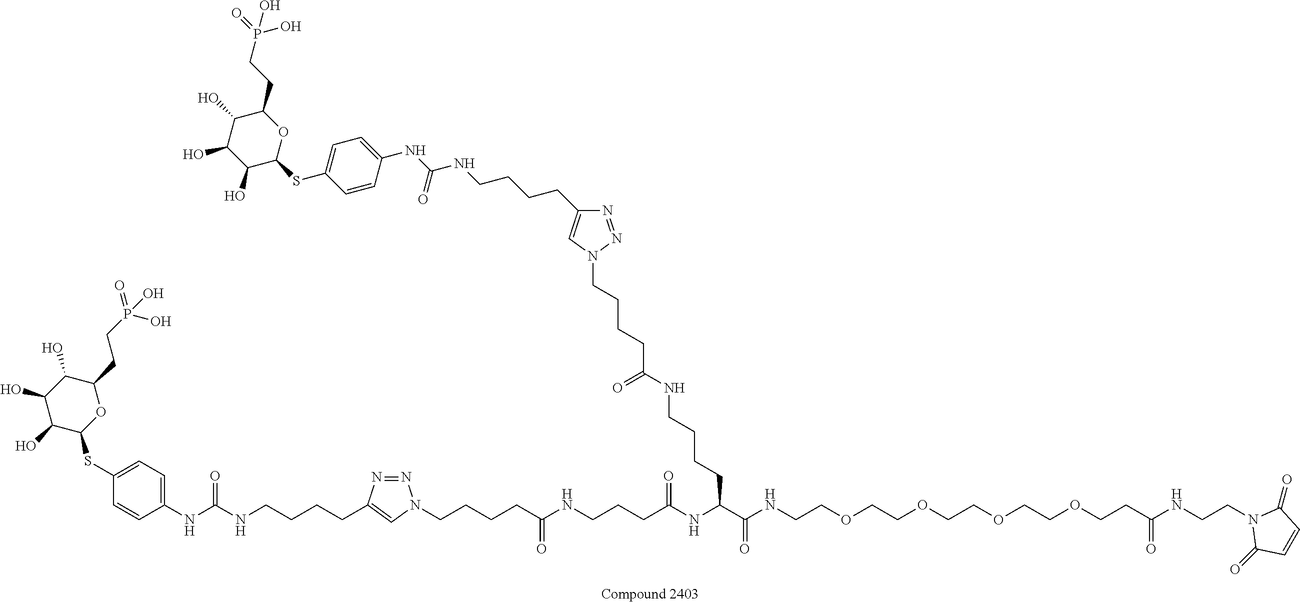 [SMILES: O=C(CCCCn1cc(CCCCNC(=O)Nc2ccc(S[C@@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(S[C@@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O]

### Synthesis of Methyl N⁶-((benzyloxy)carbonyl)-N²-(4-(((benzyloxy)carbonyl)amino)butanoyl)-L-lysinate (3)

To a vigorously stirring suspension of H-Lys(Z)—OMe hydrochloride (1, 1.00 eq, 2.00 g, 6.05 mmol), 4-(benzyloxycarbonylamino)butanoic acid (2, 1.15 eq, 1.65 g, 6.95 mmol), and HATU (1.25 eq, 2873 mg, 7.56 mmol) in DMF (4 mL) was added slowly N,N-diisopropylethylamine (3.50 eq, 3.70 mL, 21.2 mmol) and the resulting solution was stirred at room temperature for 16 h. The crude residue was purified via reverse phase chromatography (5 to 100% MeCN in water with 0.1% formic acid). Fractions containing the desired product were combined and concentrated to dryness to afford methyl N6-((benzyloxy)carbonyl)-N2-(4-(((benzyloxy)carbonyl)amino)butanoyl)-L-lysinate as a colorless oil that turns solid on hi-vac. Yield: 2.77 g, 89%; LCMS m/z 514.2 [M+1]+; 1H NMR (300 MHz, Chloroform-d) δ 7.41-7.23 (m, 1OH), 6.82 (br, J=8.3 Hz, 1H), 5.38 (br, J=6.4 Hz, 1H), 5.25 (br, 1H), 5.16-4.99 (m, 4H), 4.54 (dt, J=8.2, 4.2 Hz, 1H), 3.73-3.65 (m, 3H), 3.29-3.05 (m, 4H), 2.29-2.19 (m, 2H), 1.88-1.60 (m, 4H), 1.57-1.25 (m, 4H).

### Synthesis of Methyl (4-aminobutanoyl)-L-lysinate (4)

To a stirring solution of methyl N6-((benzyloxy)carbonyl)-N2-(4-(((benzyloxy)carbonyl)amino)butanoyl)-L-lysinate (3, 1.00 eq, 2.77 g, 5.39 mmol) in methanol (50 mL) was added 10% palladium on carbon (0.0300 eq, 172 mg, 0.162 mmol) and the resulting suspension was sparged with H2 gas for 5 min and then stirred overnight under an atmosphere of H2 gas. The reaction mixture was filtered through Celite, and the filter cake washed with MeOH and DCM. Combined filtrates were concentrated in vacuo to yield crude methyl (4-aminobutanoyl)-L-lysinate as a colorless oil/semi-solid. Used without any further purification. Yield: 1.32 g, quantitative yield; LCMS m/z 246.2 [M+1]⁺.

### Synthesis of Methyl N²-(4-(5-azidopentanamido)butanoyl)-N⁶-(5-azidopentanoyl)-L-lysinate (6)

To a stirring suspension of 5-azidopentanoic acid (5, 2.00 eq, 770 mg, 5.38 mmol) and HATU (2.10 eq, 2148 mg, 5.65 mmol) in DMF (2 mL) was added a solution of methyl (4-aminobutanoyl)-L-lysinate (4, 1.00 eq, 660 mg, 2.69 mmol) in DMF (3 mL) and the resulting yellow solution was stirred at room temperature for a couple min before adding N,N-diisopropylethylamine (3.50 eq, 1.6 mL, 9.42 mmol). The resulting orange solution was stirred for 16 h at room temperature. The reaction was diluted with EtOAc and washed with 1 N HCl, saturated sodium bicarbonate, and then brine 2×. The organics were dried over Na₂SO₄, filtered and concentrated. The crude was purified by silica gel chromatography (0 to 10% MeOH in EtOAc) to afford methyl N²-(4-(5-azidopentanamido)butanoyl)-N⁶-(5-azidopentanoyl)-L-lysinate as a light brown semi-solid. Yield: 650 mg, 49%; LCMS m/z 496.5 [M+1]+; H NMR (300 MHz, Chloroform-d) δ 6.93 (d, J=7.6 Hz, 1H), 6.31 (t, J=5.9 Hz, 1H), 6.03 (t, J=5.9 Hz, 1H), 4.52 (td, J=8.0, 4.6 Hz, 1H), 3.74 (s, 3H), 3.44-3.17 (m, 8H), 2.35-2.26 (m, 2H), 2.26-2.17 (m, 4H), 1.91-1.80 (m, 3H), 1.78-1.59 (m, 9H), 1.58-1.47 (m, 2H), 1.46-1.29 (m, 2H).

### Synthesis of N²-(4-(5-Azidopentanamido)butanoyl)-N⁶-(5-azidopentanoyl)-L-lysine (7)

To a stirring solution of methyl N²-(4-(5-azidopentanamido)butanoyl)-N⁶-(5-azidopentanoyl)-L-lysinate (6, 1.00 eq, 650 mg, 1.31 mmol) in THF (6 mL) and methanol (3 mL) was added 1 N lithium hydroxide (3.00 eq, 3.9 mL, 3.93 mmol) solution and the reaction was stirred at 45° C. for 16 h. The reaction was neutralized with 3 eq of 1N HCl, diluted with water, and extracted with DCM 3 times. Combined organics were dried and evaporated in vacuo to yield crude N²-(4-(5-azidopentanamido)butanoyl)-N⁶-(5-azidopentanoyl)-L-lysine as a colorless oil. Used without any further purification. Yield: 632 mg, quantitative yield; LCMS m/z 482.2 [M+1]+; ¹H NMR (300 MHz, Chloroform-d) δ 7.45 (d, J=7.4 Hz, 1H), 6.86 (d, J=6.3 Hz, 1H), 6.67-6.53 (m, 1H), 4.48 (s, 1H), 3.75 (tdd, J=4.2, 2.9, 1.2 Hz, 1H), 3.37-3.17 (m, 4H), 2.32 (t, J=6.2 Hz, 2H), 2.24 (t, J=7.1 Hz, 4H), 1.92-1.33 (m, 19H).

### Synthesis of tert-Butyl (S)-29-azido-18-(4-(5-azidopentanamido)butyl)-17,20,25-trioxo-4,7,10,13-tetraoxa-16,19,24-triazanonacosanoate (8)

To a stirring solution suspension of N²-(4-(5-azidopentanamido)butanoyl)-N⁵-(5-azidopentanoyl)-L-lysine (7, 1.00 eq, 632 mg, 1.31 mmol) and HATU (1.20 eq, 598 mg, 1.57 mmol) in DMF (2 mL) was added a solution of amino-PEG4-t-butyl ester (7a, 1.10 eq, 464 mg, 1.44 mmol) and N,N-diisopropylethylamine (3.00 eq, 0.69 mL, 3.93 mmol) in DMF (1.5 mL) and the resulting golden colored solution was stirred at room temperature for 16 h. The reaction mixture was diluted with EtOAc and washed with 1 N HCl, saturated sodium bicarbonate solution, and then brine. The organics were dried over Na₂SO₄, filtered, and concentrated in vacuo. The crude residue was purified by silica gel chromatography (0 to 20% MeOH in EtOAc) to afford tert-butyl (S)-29-azido-18-(4-(5-azidopentanamido)butyl)-17,20,25-trioxo-4,7,10,13-tetraoxa-16,19,24-triazanonacosanoate as a colorless semi-solid. Yield: 711 mg, 69%; LCMS m/z 785.4 [M+1]+; ¹H NMR (300 MHz, Chloroform-d) δ 7.14-7.02 (m, 2H), 6.65 (t, J=5.7 Hz, 1H), 6.39 (t, J=5.8 Hz, 1H), 4.39 (td, J=8.0, 5.1 Hz, 1H), 3.74-3.51 (m, 16H), 3.49-3.37 (m, 2H), 3.27 (dt, J=19.9, 6.8 Hz, 8H), 2.50 (t, J=6.5 Hz, 2H), 2.34-2.17 (m, 6H), 1.89-1.79 (m, 3H), 1.77-1.57 (m, 9H), 1.56-1.32 (m, 13H).

### Synthesis of (S)-29-Azido-18-(4-(5-azidopentanamido)butyl)-17,20,25-trioxo-4,7,10,13-tetraoxa-16,19,24-triazanonacosanoic acid (9)

To a stirring solution of tert-butyl (S)-29-azido-18-(4-(5-azidopentanamido)butyl)-17,20,25-trioxo-4,7,10,13-tetraoxa-16,19,24-triazanonacosanoate (8, 1.00 eq, 686 mg, 0.874 mmol) in DCM (6 mL) at 0° C. was added trifluoroacetic acid (100 eq, 6.7 mL, 87.4 mmol) and the reaction was stirred at 0° C. for 90 min. The reaction mixture was concentrated on a rotary evaporator at room temperature. The residue was diluted with DCM then concentrated multiple times. The crude residue was diluted with MeOH and purified via reverse phase chromatography (10 to 100% MeCN in water with 0.1% formic acid). Fractions containing the desired product were combined and concentrated to afford (S)-29-azido-18-(4-(5-azidopentanamido)butyl)-17,20,25-trioxo-4,7,10,13-tetraoxa-16,19,24-triazanonacosanoic acid as a light yellow oil. Yield: 636 mg, quantitative yield; LCMS m/z 729.3 [M+1]+; ¹H NMR (300 MHz, Chloroform-d) δ 7.79 (d, J=7.7 Hz, 1H), 7.65-7.49 (m, 2H), 7.46-7.36 (m, 1H), 4.51 (q, J=7.4 Hz, 1H), 3.83 (t, J=5.9 Hz, 2H), 3.77-3.61 (m, 14H), 3.56-3.45 (m, 2H), 3.33 (q, J=7.3, 6.2 Hz, 8H), 2.70 (t, J=5.9 Hz, 2H), 2.42 (t, J=7.5 Hz, 6H), 1.97-1.51 (m, 15H), 1.44-1.32 (m, 2H).

### Synthesis of Perfluorophenyl (S)-29-azido-18-(4-(5-azidopentanamido)butyl)-17,20,25-trioxo-4,7,10,13-tetraoxa-16,19,24-triazanonacosanoate (10)

To a stirring solution of (S)-29-azido-18-(4-(5-azidopentanamido)butyl)-17,20,25-trioxo-4,7,10,13-tetraoxa-16,19,24-triazanonacosanoic acid (9, 1.00 eq, 280 mg, 0.384 mmol) in THF (3 mL) was added N,N′-dicyclohexylcarbodiimide (1.50 eq, 119 mg, 0.576 mmol), 2,3,4,5,6-pentafluorophenol (9a, 1.70 eq, 120 mg, 0.653 mmol) in THF (1.5 mL) followed by DMAP (0.0300 eq, 1.4 mg, 0.0115 mmol) and the resulting clear solution was stirred at room temperature for 16 h. To the resulting suspension was added 1 mL THF, and the reaction mixture was vacuum filtered and washed with 3 mL THF. Combined filtrates were concentrated and then taken up in NMP, and then purified via reverse phase chromatography (15 to 100% MeCN in water with 0.1% formic acid). Fractions containing the desired product were combined and lyophilized to dryness to afford perfluorophenyl (S)-29-azido-18-(4-(5-azidopentanamido)butyl)-17,20,25-trioxo-4,7,10,13-tetraoxa-16,19,24-triazanonacosanoate (10) as a white solid. Yield: 313 mg, 91%; LCMS m/z 895.3 [M+1]+; H NMR (300 MHz, Chloroform-d) δ 6.86-6.70 (m, 2H), 6.20 (br, 1H), 5.98 (br, 1H), 4.44-4.32 (m, 1H), 3.88 (t, J=6.2 Hz, 2H), 3.72-3.39 (m, 16H), 3.35-3.20 (m, 8H), 2.95 (t, J=6.2 Hz, 2H), 2.33-2.14 (m, 6H), 1.99-1.01 (m, 16H).

### Synthesis of 2-[(2R,3S,4S,5S,6S)-6-[4-[4-[1-[5-[[(5S)-6-[2-[2-[2-[2-[3-[2-(2,5- dioxopyrrol-1-yl)ethylamino]-3-oxo-propoxy]ethoxy]ethoxy]ethoxy]ethylamino]-6-oxo-5-[4-[5-[4-[4-[[4-[(2S,3S,4S,5S,6R)-3,4,5-trihydroxy-6-(2-phosphonoethyl)tetrahydropyran-2-yl]sulfanylphenyl]carbamoylamino]butyl]triazol-1-yl]pentanoylamino]butanoylamino]hexyl]amino]-5-oxo-pentyl]triazol-4-yl]butylcarbamoylamino]phenyl]sulfanyl-3,4,5-trihydroxy-tetrahydropyran-2-yl]ethylphosphonic acid (Compound 2403)

A solution of (2,3,4,5,6-pentafluorophenyl) 3-[2-[2-[2-[2-[[(2S)-6-(5-azidopentanoylamino)-2-[4-(5-azidopentanoylamino)butanoylamino]hexanoyl]amino]ethoxy]ethoxy]ethoxy]ethoxy]propanoate (10) (15.2 mg, 0.0107 mmol, 1.0 eq.) in 0.5 mL of NMP was added 2-[(2R,3S,4S,5S,6S)-6-[4-(hex-5-ynylcarbamoylamino)phenyl]sulfanyl-3,4,5-trihydroxy-tetrahydropyran-2-yl]ethylphosphonic acid (10a) (18.2 mg, 0.0372 mmol, 2.2 eq.) followed by cuprous; acetonitrile; hexafluorophosphate (13.5 mg (0.0362 mmol, 2.1 eq.). The reaction solution stirred under nitrogen atmosphere at ambient temperature for 30 minutes, at which time LCMS analysis indicated formation of intermediate 11 m/z 1869.9 [M+H]. To the reaction solution was the added 2-Maleimidoethylamine hydrochloride (3.5 mg, 0.0196 mmol, 1.2 eq.) followed by N,N-diethylethanamine (7 μL, 0.050 mmol, 3.0 eq.). The reaction continued to stir under nitrogen atmosphere at ambient temperature for an additional 45 minutes, after which the reaction was diluted with DMSO and purified by reverse phase HPLC (5-50% acetonitrile in water with 0.1% TFA modifier). Fractions containing desired product were combined and lyophilized to dryness to afford 2-[(2R,3S,4S,5S,6S)-6-[4-[4-[1-[5-[[(5S)-6-[2-[2-[2-[2-[3-[2-(2,5-dioxopyrrol-1-yl)ethylamino]-3-oxo-propoxy]ethoxy]ethoxy]ethoxy]ethylamino]-6-oxo-5-[4-[5-[4-[4-[[4-[(2S,3S,4S,5S,6R)-3,4,5-trihydroxy-6-(2-phosphonoethyl)tetrahydropyran-2-yl]sulfanylphenyl]carbamoylamino]butyl]triazol-1-yl]pentanoylamino]butanoylamino]hexyl]amino]-5-oxo-pentyl]triazol-4-yl]butylcarbamoylamino]phenyl]sulfanyl-3,4,5-trihydroxy-tetrahydropyran-2-yl]ethylphosphonic acid (Compound 2403) as a white solid. Yield: 13.1 mg (42%); LCMS m/z 1827.8 [M+H].

### Synthesis of (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (10a)

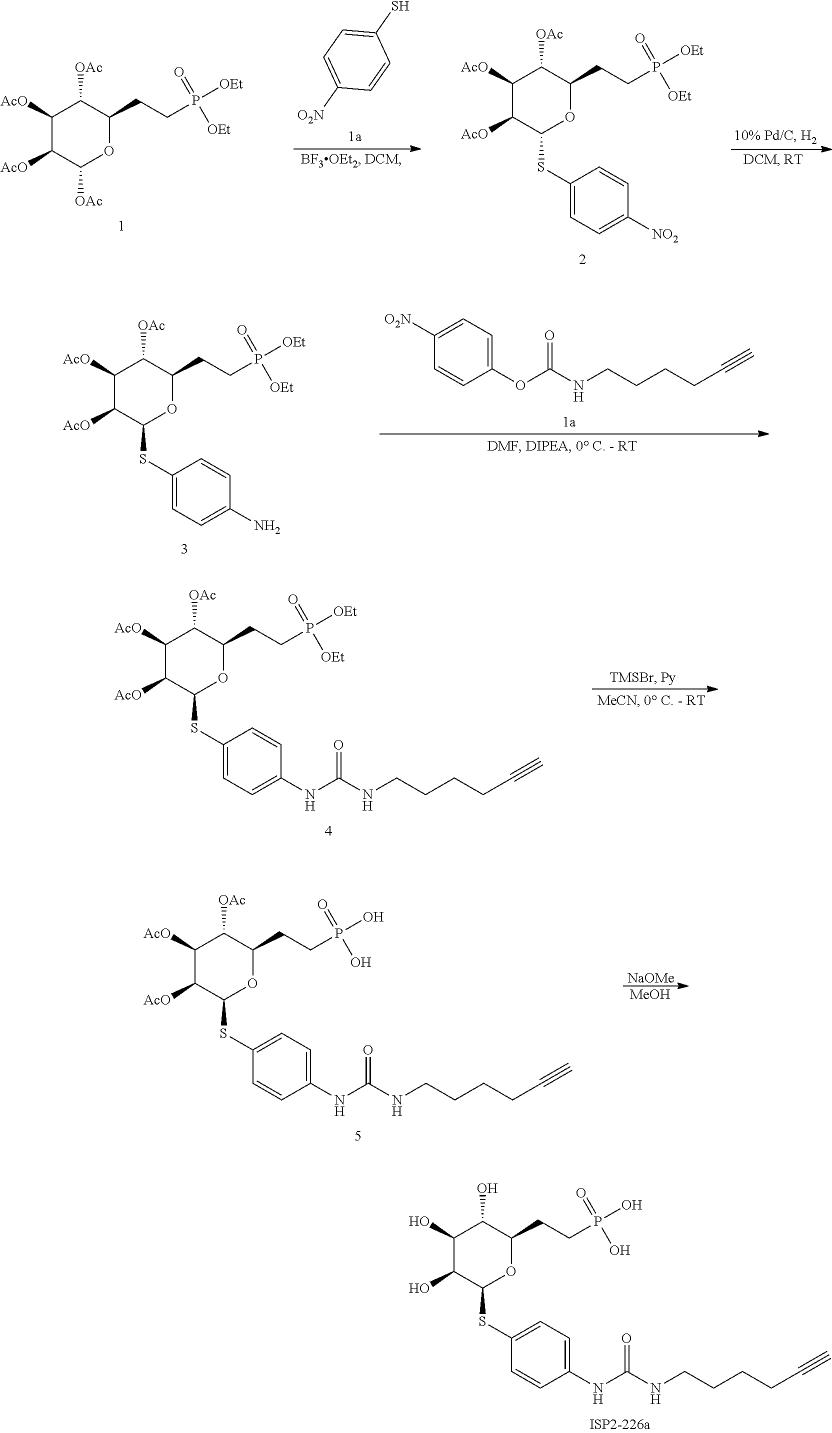

### Synthesis of (2R,3R,4S,5S,6R)-2-(2-(diethoxyphosphoryl)ethyl)-6-((4-nitrophenyl)thio)tetrahydro-2H-pyran-3,4,5-triyl triacetate (2)

To a stirred solution of (2R,3S,4S,5R,6R)-6-(2-(diethoxyphosphoryl)ethyl)tetrahydro-2H-pyran-2,3,4,5-tetrayl tetraacetate (1, 1.0 eq, 6.0 g, 12.4 mmol) and 4-nitrothiophenol (5.0 eq, 9.65 g, 62.2 mmol) in dichloromethane (80 mL), was added boron trifluoride diethyl etherate (10.0 eq, 15.2 mL, 124 mmol) at 0° C. The reaction mixture was stirred at room temperature for 16 h. After that, reaction mixture was quenched with ice water, extracted with dichloromethane. The organic layer washed with saturated bicarbonate solution, followed by water and dried over anhydrous sodium sulfate, filtered and concentrated to get crude. The crude was purified by flash column chromatography using 50-100% ethyl acetate in hexane as eluent to afford a: 0 isomer (7:3) (2R,3R,4S,5S,6R)-2-(2-(diethoxyphosphoryl)ethyl)-6-((4-nitrophenyl)thio)tetrahydro-2H-pyran-3,4,5-triyl triacetate (2) as a colorless sticky solid. Yield: 4.0 g, 55.7%; LCMS, m/z. 578.14 [M+1]⁺.

### Synthesis of (2R,3S,4S,5R,6R)-2-((4-aminophenyl)thio)-6-(2-(diethoxyphosphoryl)ethyl)tetrahydro-2H-pyran-3,4,5-triyl triacetate (3)

To the stirred solution of (2R,3R,4S,5S,6R)-2-(2-(diethoxyphosphoryl)ethyl)-6-((4-nitrophenyl)thio)tetrahydro-2H-pyran-3,4,5-triyl triacetate (2, 1.0 eq, 1.2 g, 2.08 mmol) in dichloromethane (15.0 mL), 10% Palladium on carbon (0.62 g, 50% w/w) were added and reaction mixture was stirred under hydrogen (balloon pressure) at room temperature for 16 h. The progress of reaction was monitored by LC-MS and TLC. After the completion of reaction, reaction mixture was filtered through syringe filter. The filtrate was concentrated under reduced pressure bath temperature <35° C.) to afford crude mixture of a: isomer (7:3) (2R,3S,4S,5R,6R)-2-((4-aminophenyl)thio)-6-(2-(diethoxyphosphoryl)ethyl)tetrahydro-2H-pyran-3,4,5-triyl triacetate (a isomer) and (2R,3S,4S,5R,6R)-2-((4-aminophenyl)thio)-6-(2-(diethoxyphosphoryl)ethyl)tetrahydro-2H-pyran-3,4,5-triyl triacetate (P isomer). The crude mixture was purified by prep-HPLC using (10-35% MeCN in water with 0.1% TFA). Fractions containing the desired product were combined and lyophilized to dryness to afford (2R,3S,4S,5R,6R)-2-((4-aminophenyl)thio)-6-(2-(diethoxyphosphoryl)ethyl)tetrahydro-2H-pyran-3,4,5-triyl triacetate (3) as off white solid. Yield: 0.2 g, 18%, f isomer LCMS, m/z. 547.97 [M+1]⁺.

### Synthesis of (2R,3R,4S,5S,6S)-2-(2-(diethoxyphosphoryl)ethyl)-6-((4-(3-(hex-5-yn-1-yl)ureido)phenyl)thio)tetrahydro-2H-pyran-3,4,5-triyl triacetate (4)

To a solution of (2S,3S,4S,5R,6R)-2-((4-aminophenyl)thio)-6-(2-(diethoxyphosphoryl)ethyl)tetrahydro-2H-pyran-3,4,5-triyl triacetate (3, 1.0 eq, 1.04 g, 1.90 mmol) in N,N-dimethyl formamide (12.0 mL), N,N-diisopropylethyl amine (2.0 eq, 0.663 mL, 3.80 mmol) and 4-nitrophenyl hex-5-yn-1-ylcarbamate (1a, 2.0 eq, 0.996 g, 3.80 mmol) were added. The reaction mixture was stirred at room temperature for 16 h. The progress of reaction was monitored by LCMS. The reaction mixture was concentrated under reduced pressure to afford crude. The crude was purified by reverse phase (Aq C-18 column) column chromatography using 20-50% acetonitrile in water as eluent. The fractions were washed with ethyl acetate. The organic layer dried over anhydrous sodium sulphate, filtered and concentrated under reduced pressure to afford (2R,3R,4S,5S,6S)-2-(2-(diethoxyphosphoryl)ethyl)-6-((4-(3-(hex-5-yn-1-yl)ureido)phenyl)thio)tetrahydro-2H-pyran-3,4,5-triyl triacetate (4) as brown sticky solid. Yield: 0.65 g (52.59%) LCMS m/z. 671.22 [M+1]⁺.

### Synthesis of (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (5)

To a stirred solution of (2R,3R,4S,5S,6S)-2-(2-(diethoxyphosphoryl)ethyl)-6-((4-(3-(hex-5-yn-1-yl)ureido)phenyl)thio)tetrahydro-2H-pyran-3,4,5-triyl triacetate (4, 1.0 eq, 0.25 g, 0.373 mmol) in dichloromethane (8.0 mL), pyridine (10.0 eq, 0.30 mL, 3.73 mmol) cooled to 0° C. and bromotrimethylsilane (10.0 eq, 0.49 mL, 3.73 mmol) was added and reaction mixture was stirred at room temperature for 16 h. After completion, reaction mixture was quenched with ice water, extracted with dichloromethane. The organic layer separated, dried over anhydrous sodium sulfate, filtered and concentrated under reduced pressure to get off-white solid. It was further washed with diethyl ether and dried to afford (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (5) as off white solid. Yield: 0.16 g (69.84%) LCMS m/z. 614.93 [M+1]⁺.

### Synthesis of (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (10a)

To the stirred solution of (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (3, 1.0 eq, 0.08 g, 0.142 mmol) in methanol (3 mL), sodium methoxide 25 w/v in methanol (7.0 eq, 0.21 mL, 0.991 mmol) was added drop-wise to this solution and reaction mixture was allowed to stir at room temperature. The progress of the reaction was monitored by LCMS. After 2 h, reaction mixture was neutralized with Dowex-hydrogen from (200-400 mesh) (up to pH-7). The reaction mixture was filtered, concentrated under reduced pressure to get crude. The crude was purified by prep-HPLC using (50-80% acetonitrile in water with 0.1% TFA). Fractions containing the desired product were combined and lyophilized to dryness to afford (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (10a) as white solid. Yield: 0.016 g, 23.12%; LC-MS m/z. 489.17 [M+1]⁺. ¹H NMR (400 M+Hz, DMSO-d₆) δ 8.47 (s, 1H), 7.34 (d, J=8.8 Hz, 2H), 7.25 (d, J=8.4 Hz, 2H), 6.16 (t, J=5.6 Hz, 1H), 4.93 (bs, 1H), 4.78 (s, 2H), 3.81 (s, 1H), 3.31 (dd, J=3.2, 9.2 Hz, 1H), 3.22 (t, J=9.2 Hz, 1H), 3.08 (dd, J=6.0, 11.6 Hz, 2H), 3.02-2.97 (m, 1H), 2.77 (t, J=2.8 Hz, 1H), 2.20-2.16 (M, 2H), 2.07-1.99 (m, 1H), 1.78-1.67 (m, 1H). 1.54-1.41 (m, 6H).

The following compounds were prepared according to the procedures disclosed above using the starting amines disclosed above via the general methods indicated.

| Compound | Starting amine used | LCMS m/z |
|---|---|---|
| No. | and Synthesis route | observed |
| 2324 | Method D, from XB91 | 1902.6 [M + H] |
| 2307 | Provided, from XB48 | 1770.4 [M + H] |
| 2308 | Analogous to Cmpd 2307, | 1453.5 [M + H] |
| | from XB133 and 2- | |
| | bromoacetic anhydride | |
| 2311 | Analogous to Cmpd 2325, | 940.2 [M + 2H]⁺⁺ |
| | from XB85 | |
| 2325 | Provided | 1888.2 [M + H] |
| 2310 | analogous to Cmpd 2325, | 971.5 [M + 2H]⁺⁺ |
| | from XB82 | |
| 2309 | Method D, from XB94 | 1065.9 [M + 2H]⁺⁺ |
| 2312 | Method D, from XB92 | 1168.26 [M + 2H]⁺⁺ |
| 2329 | Method D, from XB97 | 1929.6 [M + H] |
| 2306 | Method D, from XB103 | 1902.6 [M + H] |
| 2326 | Method D, from XB83 | 1027.2 [M + 2H]⁺⁺ |
| 2328 | Method D, from XB57 | 1932.6 [M + H] |
| 2327 | Method D, from XB80 | 1039.0 [M + 2H]⁺⁺ |
| 2323 | Method D, from XB50 | 1954.4 [M-H]⁻ |
| 2313 | Method D, from XB48 | 1771.1 [M + H] |
| 2322 | Analogous to Cmpd 2314, | 1763.4 [M + H] |
| | from Cmpd 2316-Int2 | |
| 2314 | Provided | 1749.5 [M + H] |
| 2316 | Provided | 1685.7 [M + H] |
| 2321 | Provided | 1671.7 [M + H] |
| 2315 | Provided | 1785.8 [M + H] |
| 2320 | Analogous to Cmpd 2321 | 1252.4 [M + H] |
| 2318 | provided | 1329.5 [M + H] |
| 2319-C | Provided | 856.4 [M + H] |
| 2317 | Provided | 1908.5 [M + H] |
| 2303 | Method D, from XB87 | 1878.74 [M + H] |
| 2304 | Analogous to Cmpd 2318, | 1554.3 [M + H] |
| | from XB80 | |
| 2305 | Method E, from XB84 | 1926.5 [M + H] |
| 2401-C | Provided | 1751.0 [M + H] |
| 2402-C | Provided | 1919.5 [M + H] |
| 2301 | Method D, from XB108 | 1102.2 [M + 2H]⁺⁺ |
| 2302 | Method D, from XB109 | 1123.2 [M + 2H]⁺⁺ |
| 1248 | Provided | 1804.7 [M + H] |
| 1255 | Provided, from XB54 | 1910.6 [M + H] |
| 1251 | provided, Method C, | 1753.7 [M + H] |
| | from XB47 | |
| 2330 | Method D, from XB100 | 1005.8 [M + 2H]⁺⁺ |
| 2343 | Analogous to Cmpd 2314, | 1923.4 [M + H] |
| | from XB47 | |
| 2346 | Method F, from XB130 | 1948.6 [M + H] |
| 2355 | Method B, from XB83 | 1032.9 [M + 2H]⁺⁺ |
| 1228 | analogous to I-168, | 1912.6 [M + H] |
| | from XB4B | |
| 2403 | Provided | 1827.8 [M + H] |
| 1117 | Provided | 881.2 [M + H]⁺ |
| (I-168) | | |

Conjugates referred to by a number (e.g. 1226, 1228, etc.) are understood to reference a conjugate described herein where the corresponding compound with a chemoselective ligation group (e.g. 1226, 1228, etc.) has been conjugated to the designated target-binding moiety. For example, “Oma-1226,” “Oma-1226 conjugate” and similar refers to the compound having a chemoselective ligation group 1226 conjugated to omalizumab.

### Example 1: Preparation of Antibody Conjugates

### Conjugation of Isothiocyanate-Based Ligand-Linker Compounds with Antibodies

This example provides a general protocol for the conjugation of the isothiocyanate-based ligand-linker compounds with the primary amines on lysine residues of antibodies.

The antibody was buffer exchanged into 100 mM sodium bicarbonate buffer pH 9.0 at 5 mg/mL concentration, after which about 30 equivalents of the isothiocyanate-based ligand-linker compound (e.g., freshly prepared as 20 mM stock solution in DMSO) was added and incubated overnight at ambient temperature in a tube revolver at 10 rpm.

The conjugates were purified using a PD-10 desalting column (GE Healthcare) and followed by formulating the final conjugate into PBS pH 7.4 with Amicon Ultra 15 mL Centrifugal Filters with 30 kDa molecular weight cutoff.

### Conjugation of Perfluorophenoxy-Based Ligand-Linker Compounds with Antibodies

This example provides a general protocol for the conjugation of the perfluorophenoxy-based ligand-linker compounds with the primary amines on lysine residues of antibodies.

The antibody was buffer exchanged into 50 mM sodium phosphate buffer pH 8.0 at 5 mg/mL concentration, after which about 22 equivalents of perfluorophenoxy-based ligand-linker compound (e.g., freshly prepared as 20 mM stock solution in DMSO) was added and incubated for 3 hours at ambient temperature in a tube revolver at 10 rpm.

The conjugates containing on average four ligand-linker moieties per IgG antibody are purified using a PD-10 desalting column (GE Healthcare) and followed with formulating the final conjugate into PBS pH 7.4 with Amicon Ultra 15 mL Centrifugal Filters with 30 kDa molecular weight cutoff.

### Determination of DAR Values by Mass Spectrometry

This example provides the method for determining DAR values for the conjugates prepared as described above. To determine the DAR value, 10 μg of the antibody (unconjugated or conjugated) was treated 2 μL of non-reducing denaturing buffer (10×, New England Biolabs) for 10 minutes at 75° C. The denatured antibody solution was then deglycosylated by adding 1.5 μL of Rapid-PNGase F (New England Biolabs) and incubated for 10 minutes at 50° C. Deglycosylated samples were diluted 50-fold in water and analyzed on a Waters ACQUITY UPLC interfaced to Xevo G2-S QToF mass spectrometer. Deconvoluted masses were obtained using Waters MassLynx 4.2 Software. DAR values were calculated using a weighted average of the peak intensities corresponding to each loading species using the formula below:

$$DAR = \sum ( drug load distribution ( \% ) of each Ab with drug load n ) ( n ) / 100$$

### Determination of Purity of Conjugates by SEC Method

Purity of the conjugates prepared was determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL was loaded to a TSKgel SuperSW3000 column, at a constant flow rate of 0.35 mL/min. Chromatographs were integrated based on elution time to calculate the purity of monomeric conjugate species.

### Antibody Disulfide Reduction and Thiol-Reactive Ligand-Linker Conjugation to Antibody

This example provides an exemplary protocol for reduction of the disulfides of the antibodies described herein, and/or conjugation of the reduced antibodies to thiol-reactive ligand-linker compounds described herein, e.g., containing a maleimide chemoselective ligation group.

### Protocol: Antibody Disulfide Reduction

A) Diluted antibody to 15 mg/mL (0.1 mM IgG) in PBS, pH 7.4.
B) Prepared a fresh 20 mM (5.7 mg/mL) stock solution of tris(2 carboxyethyl)phosphine (TCEP) in H₂O.
C) Added 25 μL of TCEP stock solution from step B) above to 1 mL of antibody from step A) above (0.5 mM final concentration TCEP).
D) Incubated at 37° C. for 2 hours (checked for free thiols using 5,5′-dithiobis-(2-nitrobenzoic acid) (DTNB) test).
E) Aliquoted the reduced antibody into 4 tubes (250 μL each).

### Ligand-Linker Conjugation to Antibody

A) Prepared 10 mM stock solution of thiol-reactive ligand-linker compound in DMSO (DMA, DMF or CH₃CN are also acceptable).
B) Added 5 equivalents of 12.5 μL stock solution from step A) above to each tube of reduced antibody (0.5 mM final concentration ligand-linker compound stock solution).
C) Incubated overnight at 4° C. for 4 hours at room temperature; checked for free thiols using DTNB test.
D) Ran analytical hydrophobic interaction chromatography (HIC) to determine DAR and homogeneity.

### Alternative Site-Specific Conjugation of Antibodies with Thiol-Reactive Ligand-Linker Compounds

The engineered cysteines of the purified antibodies were cysteinylated, which is a common process-related modification. These thiol “caps” render the engineered cysteines unreactive and need to be removed before conjugation to thiol-reactive compounds. This example provides an exemplary protocol for “de-capping” of the engineered cysteines and conjugation to the thiol-reactive compounds described herein, e.g., containing a maleimide or bromoacetamide reactive group.

### Protocol:

1. Reduction using tris(2-carboxyethyl)phosphine (TCEP): Buffer exchanged the antibody into phosphate-buffered saline (PBS) pH 7.2 using a desalting column (e.g. Cytiva 26/10 desalting column or Zeba™ Spin Desalting Column), and added 20 molar equivalents of 0.5 M TCEP (Bond-Breaker™ TCEP Solution, Thermo Scientific™) to the antibody. Incubated at 37° C. for 1.5 hours or room temperature for 2-3 hours.
   2. Re-Oxidation Using Dehydroascorbic Acid (dhAA):
a) Removed excess TCEP from the reduced antibody using a desalting column (e.g. Cytiva 26/10 desalting column or Zeba™ Spin Desalting Column) equilibrated with 100 mM HEPES (4-(2-hydroxyethyl)-1-piperazineethanesulfonic acid), 50 mM NaCl, 1 mM EDTA (ethylenediaminetetraacetic acid), pH 7.2 (HBSE buffer).
b) Determined concentration of the reduced antibody using UV absorbance measurements on a ThermoFisher Nanodrop instrument and the extinction coefficient calculated from the antibody's primary amino acid sequence.
c) Prepared a fresh stock solution of 80 mM dhAA in HBSE.
d) Added 40 molar equivalents of dhAA to reduced antibody and incubated at room temperature for 2-16 hours.
e) Buffer exchanged into conjugation buffer (100 mM Borate, 50 mM NaCl, 1 mM EDTA, pH 8.3) (BBSE) using a desalting column.
f) Filtered using a 0.2-micron syringe filter.
g) Determined concentration of “de-capped” antibody using UV absorbance measurements on a ThermoFisher Nanodrop instrument and the extinction coefficient calculated from the antibody's primary amino acid sequence. h) Aliquoted, froze and stored in −80° C. until conjugation.

### 3. Conjugation—Reaction Setup and Purification:

a) Thawed de-capped antibody and pre-warmed at 37° C. for 10-15 minutes.
b) Prepared 25-30 mM stock solution of the thiol-reactive compound in N, N-dimethylacetamide (DMA) (DMSO, DMF or CH₃CN are also acceptable).
c) Added 5 molar equivalents of maleimide-functionalized compounds and 8 molar equivalents of bromo-functionalized compounds into the de-capped antibody (2.5 and 4 molar equivalents per engineered cysteine, respectively).
d) Incubated at 37° C. for 4 hours.
e) Purified by size exclusion chromatography using a Superdex 200 Increase 10/300 GL column (Cytiva) and PBS pH 7.5.
f) Pooled desired fractions and filtered using a 0.2-micron syringe filter.

The above protocol describes conjugation of the exemplary antibodies with the maleimide-reactive compounds at pH 8.3 and 37° C. Alternatively, the conjugation reaction can be performed at room temperature for 1-2 hours in the HBSE buffer used for the re-oxidation step (pH 7.2), and subsequently buffer exchanged into the BBSE buffer (pH 8.3) and incubated at 37° C. for 4-18 h to facilitate conjugate stabilization through hydrolysis of the maleimide ring.

### Conjugation of Thiol-Reactive Ligand-Linker Compounds to the Endogenous Cysteines of Antibodies

This example provides a general protocol for reduction of the interchain disulfides of antibodies, and conjugation of the reduced antibodies with the thiol-reactive ligand-linker compounds described herein, e.g., containing a maleimide reactive group.

### Protocol:

1. Reduction using tris(2-carboxyethyl)phosphine (TCEP): Buffer exchanged the antibody into PBS pH 7.2 using a desalting column (e.g. Cytiva 26/10 desalting column or Zeba™ Spin Desalting Column), and added 20 molar equivalents of 0.5 M TCEP (Bond-Breaker™ TCEP Solution, Thermo Scientific™) to the antibody. Incubated at 37° C. for 1.5 hours or room temperature for 2-3 hours.

### 2. Conjugation—Reaction Set-Up and Purification:

a) Removed excess TCEP from the reduced antibody using a desalting column (e.g. Cytiva 26/10 desalting column or Zeba™ Spin Desalting Column) equilibrated with 100 mM Borate, 50 mM NaCl, 1 mM EDTA, pH 8.3 (BBSE) buffer.
b) Determined concentration of the reduced antibody using UV absorbance measurements on a ThermoFisher Nanodrop instrument and the extinction coefficient calculated from the antibody's primary amino acid sequence.
c) Prepared 25-30 mM stock solution of the thiol-reactive compound in DMA (DMSO, DMF or CH₃CN are also acceptable).
d) Added 14 molar equivalents (1.75 molar equivalents per free thiol for a typical IgG1) of maleimide-functionalized compounds into the reduced antibody.
e) Incubated at 37° C. for 4 hours.
f) Purified by size exclusion chromatography using a Superdex 200 Increase 10/300 GL column (Cytiva) and PBS pH 7.5.
g) Pooled desired fractions and filtered using a 0.2-micron syringe filter.

The above protocol describes conjugation of antibodies with the maleimide-reactive compounds at pH 8.3 and 37° C. Alternatively, the conjugation reaction can be performed at room temperature for 1-2 hours in 100 mM HEPES (4-(2-hydroxyethyl)-1-piperazineethanesulfonic acid), 50 mM NaCl, 1 mM EDTA, pH 7.2, and subsequently buffer exchanged into the BBSE buffer (pH 8.3) and incubated at 37° C. for 4-18h.

### Conjugation of Pentafluorophenyl (PFP) Ester- Based Ligand-Linker Compounds with Antibodies

This example provides a general protocol for the conjugation of the PFP-based ligand-linker compounds with the primary amines on lysine residues of antibodies.

The antibody was buffer exchanged into 100 mM Borate, 50 mM NaCl, 1 mM EDTA, pH 8.3 (BBSE), after which about 6-7 equivalents of PFP-based ligand-linker compound (e.g., freshly prepared as 20-30 mM stock solution in DMA) was added and incubated for 3-18 hours at ambient temperature. The conjugates were subsequently purified using size exclusion chromatography (e.g. Superdex 200 Increase 10/300 GL column, Cytiva) and PBS pH 7.5.

### Characterization of Final Conjugates

The conjugates, which were prepared as described above, were quantified, and characterized as follows:
a) Concentration: The concentration of the conjugates was determined using the bicinchoninic acid assay following manufacturer's instructions (Pierce™ BCA protein assay kit) and the unconjugated exemplary antibody as standard.
b) Endotoxin content: The conjugates were diluted to 1 mg/mL with water and the endotoxin content was measured using Limulus amebocyte lysate cartridges (Charles River).
c) Aggregate analysis: The aggregation profile of the conjugates was evaluated by size exclusion high performance liquid chromatography (SEC-HPLC). The conjugates were diluted to 1 mg/mL with PBS and 10 μL were loaded onto a Zenix SEC-300 column (Sepax) using isocratic elution at a constant flow rate of 0.5 mL/min and 2×PBS as the mobile phase. Chromatographs were integrated based on elution time to calculate the purity of monomeric conjugate species. All conjugates described herein were >95% monomeric.
d) Degree of conjugation (DoC): The identity and DoC were determined via liquid chromatography-mass spectrometry (LC-MS). For that purpose, 15 g of the antibody (unconjugated or conjugated) were diluted to 1 mg/mL with PBS, and deglycosylated by addition of 0.6 μL of Rapid-PNGase F (New England Biolabs) and incubation at 50° C. for 10 minutes. The deglycosylated samples were diluted 10-fold in 0.2% formic acid and analyzed on a Waters ACQUITY UPLC interfaced to Xevo G2-S QToF mass spectrometer. Deconvoluted masses were obtained using Waters MassLynx 4.2 Software and DoC values were calculated using a weighted average of the peak intensities corresponding to each conjugate species.

Unless indicated otherwise, all cysteine conjugates to omalizumab (Oma) and ligelizumab (Lige) were conjugated at L443C. Other information about the Oma and Lige used may be found herein.

### Example 2: Assessing IgE Clearance by Omalizumab (Oma) Conjugates In Vivo

In vivo studies were performed to assess the ability of Oma conjugates to clear human IgE (hIgE) from serum that was dosed approximately 24 hours prior to administration of the Oma conjugate. Briefly, female C57BL/6 mice were dosed intravenously with 1 mg/kg of hIgE, followed by intravenous dosing of 5 mg/kg of either Oma or Oma-1226 the following day. Serum samples were collected immediately prior to administration of the Oma conjugate and at 1 hour, 4 hours, and 24 hours following administration. Three mice were in each experimental group. Serum hIgE was measured by Meso Scale Discovery immunoassay.

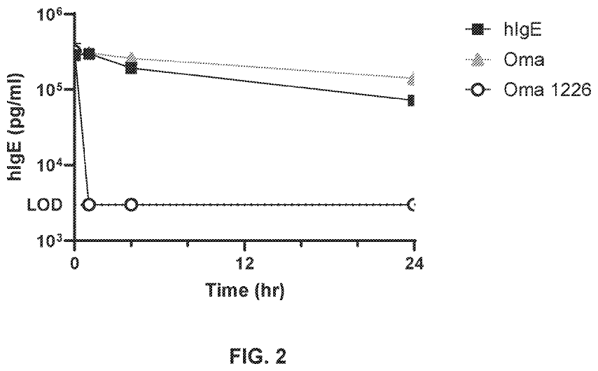

Results shown in FIG. 2 demonstrate that by one hour following administration of the Oma conjugate Oma- 1226, the 1 mg/kg dose of hIgE dosed the day before had been cleared. In contrast, Oma which contained the site-specific Leucine to Cysteine mutation at Oma residue 443 but was not conjugated to a compound, appeared to stabilize the dosed hIgE in mouse serum.

Additional in vivo studies were performed to assess the ability of Oma conjugates to clear hIgE from serum that was dosed approximately 24 hours after administration of the Oma conjugate. Briefly, female C57BL/6 mice were dosed intravenously with 5 mg/kg of either Oma; Oma-1226; or Oma-1228, followed by intravenous dosing of 1 mg/kg hIgE the following day. Serum samples were collected at 5 minutes, 1 hour, 4 hours, and 24 hours following administration of hIgE. Three mice were in each experimental group. Serum hIgE was measured by Meso Scale Discovery immunoassay.

Results shown in FIG. 3 demonstrate that the Oma conjugate Oma-1226 lost substantial activity within 24 hours of administration. In contrast, results shown in FIG. 4 demonstrated that the Oma conjugate Oma-1228 retained sufficient activity 24 hours after administration to completely clear hIgE dosed at 1 mg/kg.

### Example 3: Assessing Clearance of Super-Stoichiometric Quantities of IgE by Oma Conjugates in Vivo

In vivo studies were performed to assess the ability of Oma conjugates to clear super-stoichiometric quantities of hIgE (1:10 Oma conjugate:hIgE). Briefly, female C57BL/6 mice were dosed intravenously with 0.5 mg/kg of Oma; Oma-1117 (L443C conjugate); or Oma-1119 (DAR 4 lysine conjugate), followed by 5 mg/kg of hIgE the next day. Serum samples were collected at 5 minutes, 1 hour (Oma; Oma-1117; and hIgE treatment conditions only), 4 hours, and 24 hours following administration of hIgE. Three mice were in each experimental group. Serum IgE concentration was measured by Meso Scale Discovery immunoassay.

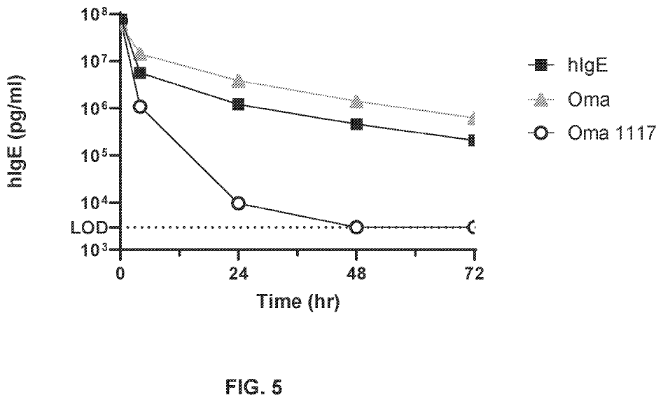

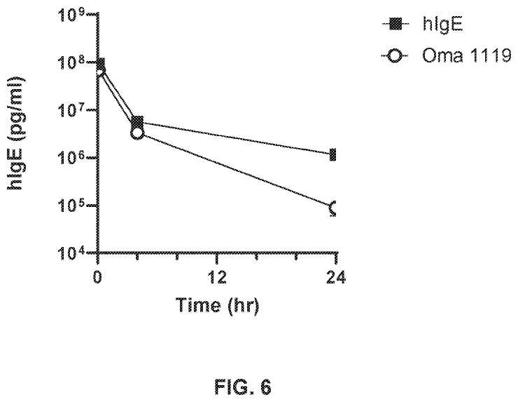

Results shown in FIG. 5 demonstrated that the Oma conjugate Oma-1117 retained sufficient activity after 24 hours to clear super-stoichiometric quantities of IgE within 48 hours. Similarly, results shown in FIG. 6 demonstrated that Oma-1119 (DAR 4 lysine conjugate) also retained sufficient activity after 24 hours to clear super-stoichiometric quantities of hIgE within 48 hours of hIgE dosing.

### Example 4: Assessing Efficacy of Oma Conjugates Following Repeat Dosing of Target Molecule

In vivo studies were performed to assess the efficacy of Oma conjugates following repeat dosing of hIgE. Briefly, female C57BL/6 mice were dosed intravenously with 5 mg/kg of Oma or Oma-1117. 24 hours later, hIgE was administered intravenously at a dose of 5 mg/kg, followed by a second identical dose of hIgE 48 hours after administration of the first dose. Serum samples were collected at 5 minutes, 4 hours, and 24 hours after each of the first and second administrations of hIgE. Three mice were in each experimental group. Serum hIgE concentrations were measured by Meso Scale Discovery immunoassay.

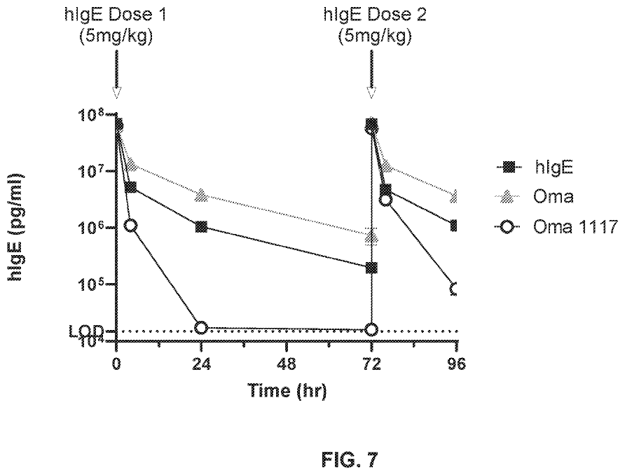

Results shown in FIG. 7 demonstrated that a single 5 mg/kg dose of Oma-1117 remained active long enough to clear two separate 5 mg/kg doses of hIgE administered over a 72 hour period.

### Example 5: Evaluating Whether Sustained Activity of Oma Conjugates is Dependent on Functional FcRn

In vivo studies were performed to evaluate whether sustained activity of Oma conjugates is dependent on functional FcRn. Briefly, female C57BL/6 and female FcRn−/− mice were administered 0.5 mg/kg of either Oma or Oma-1120 intravenously, followed by intravenous administration of 5 mg/kg hIgE the following day. Serum samples were collected at 5 minutes, 4 hours, and 24 hours following administration of hIgE. Three mice were in each experimental group. Serum IgE concentrations were measured by Meso Scale Discovery immunoassay.

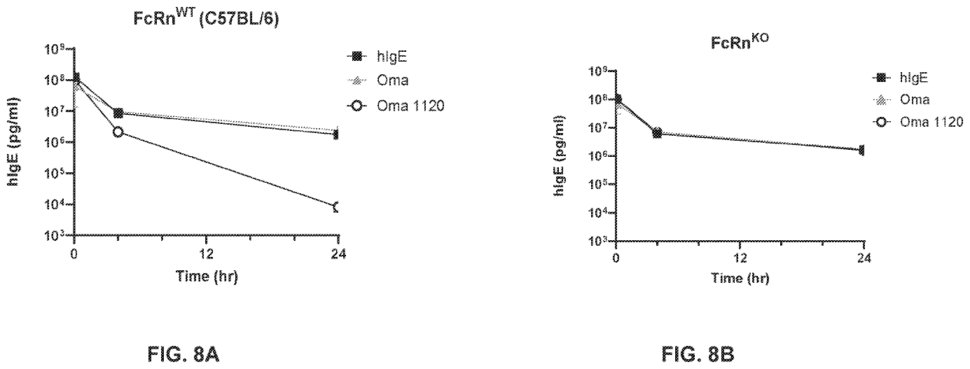

The results shown in FIG. 8A (FcRn wild-type mice) and FIG. 8B (FcRn knockout mice) demonstrated that extended activity and super-stoichiometric clearance of hIgE observed for the Oma conjugate Oma-1120 was dependent on the presence of functional FcRn.

The following experiments were performed with HepG2 hepatocellular carcinoma cells, grown in Eagle's Minimal Essential Media (EMEM) supplemented with 10% FBS and penicillin/streptomycin.

### Example 6: Internalization of Conjugates in Cells (Uptake Assay)

HepG2 cells (25,000) were plated in 96-well plates 48 hours before the experiment was performed. On the day of the experiment, indicated the conjugate was mixed with an equimolar stock of Alexa Fluor647 labelled human IgE (Enzo Life Sciences, BPD-DIA-HE 1) in fresh EMEM as a 600 nM stock. The precomplexed conjugate:IgE mixture was incubated at room temperature for 30 minutes. During the precomplexing step, media from HepG2 was removed, and 40 ul of EMEM with 1/100 Fc Receptor Blocking Solution (Biolegend Human TruStain FcX, 422302) was added. Cells incubated in the receptor blocking solution for 10 minutes while making serial dilutions (1:3) of conjugate:IgE stock solution in EMEM. 40 ul of the conjugate:IgE mixture in EMEM was then added to cells (six dilutions, with final concentrations ranging from 300 nM to 1.23 nM) for 2 hours before washing with DPBS and detaching with 0.25% Trypsin/EDTA. Cells were pelleted in 96-well conical bottom plates, washed once with FACS Buffer with BSA (Rockland Immunochemicals), and resuspended in FACS Buffer with BSA. Uptake of fluorescently labelled IgE was measured by analyzing median cellular AF647 intensity by flow cytometry.

Table 27 shows uptake (cellular internalization) of Alexa Fluor 647 labelled IgE in wild type and ASGPR1/2 KO HepG2 cells treated with the conjugate. Cells were incubated with equimolar ratio of conjugate and AF647 IgE at indicated concentrations, with data normalized to unconjugated parent protein (Oma S35HWY57H-LC). No significant increase in uptake was observed in ASGPR1/2 KO cells versus the unconjugated control.

TABLE 27

| Oma | Normalized Fluorescence | | |
|---|---|---|---|
| S35H/ | | | 100 nM |
| Y57H-LC | 100 nM | 3.7 nM | ASGPR |
| Conjugate | WT | WT | KO |
| Unconjugated | 1.0 | 1.0 | 1.0 |
| 2318 | 159.1 | 44.1 | 1.0 |
| 2323 | 252.2 | 76.6 | 1.1 |
| 2328 | 146.5 | 29.7 | 1.1 |
| 2326 | 287.9 | 89.7 | 1.1 |
| 2306 | 164.6 | 31.0 | 1.1 |
| 2325 | 270.0 | 87.8 | 1.2 |
| 2313 | 259.1 | 89.1 | 1.0 |
| 2327 | 268.8 | 133.1 | 1.0 |
| 2317 | 255.2 | 76.2 | 1.1 |
| 2314 | 265.7 | 69.0 | 1.0 |
| 2315 | 275.7 | 77.7 | 1.1 |
| 2316 | 314.3 | 68.4 | 1.1 |
| 2321 | 320.7 | 80.7 | 1.1 |
| 2320 | 224.4 | 37.7 | 1.1 |
| 2329 | 201.8 | 56.3 | 1.1 |
| 2330 | 166.6 | 54.5 | 1.1 |
| 2310 | 127.9 | 30.3 | 1.1 |
| 2311 | 92.9 | 22.6 | 1.1 |
| 2401-C | 44.7 | 10.3 | 1.2 |
| 2402-C | 84.3 | 19.6 | 1.3 |
| 2322 | 214.4 | 68.5 | 1.2 |
| 2319-C | 16.0 | 2.3 | 1.4 |
| 2312 | 242.9 | 64.9 | 1.0 |
| 2308 | 261.4 | 55.1 | 1.1 |
| 2305 | 233.4 | 64.8 | 1.1 |
| 2304 | 292.5 | 150.3 | 1.0 |
| 2303 | 232.7 | 70.6 | 1.1 |
| 2309 | 201.6 | 38.9 | 1.1 |
| 2307 | 253.2 | 81.6 | 1.2 |
| 2324 | 207.4 | 39.2 | 1.2 |

### Example 7: Lysosomal Degradation of Target

HepG2 cells (15,000) were plated in 96-well plates 72 hours before performing the experiment. On the day of the experiment, indicated the conjugate was diluted in fresh EMEM and mixed with an equimolar stock of LysoLight-labelled human IgE (Enzo Life Sciences, BPD-DIA-HE1), at 200 nM of each compound. The precomplexed conjugate:IgE mixture was incubated at room temperature for 30 minutes. During the precomplexing step, media from HepG2 was removed, and 40 ul of EMEM with 1/100 Fc Receptor Blocking Solution (Biolegend Human TruStain FcX, 422302) was added. Cells incubated in the receptor blocking solution for 10 minutes while making serial dilutions (1:3) of conjugate:IgE stock solution in EMEM. 40 ul of the conjugate:IgE mixture in EMEM was then added to cells (five dilutions, with final concentrations ranging from 100 nM to 1.23 nM). Cells were placed in an IncuCyte Live-Cell Analysis System (Sartorius) for long term live cell imaging analysis (10× magnification, 3 images per well). One hour after addition of compound, image acquisition to measure red fluorescence from the LysoLight probe was started, with further Images collected every hour for 72 hours. Fluorescence of the LysoLight probe only occurs when cleaved by lysosomal cathepsin proteases, confirming lysosomal delivery and degradation of target. After 72 hours, mean red fluorescence was measured for each time point, and the area under the curve of fluorescence overtime was used to measure total degradation over the 3-day time period.

Table 28 shows lysosomal degradation of target in the conjugate treated cells tracked with IgE conjugated to a quenched cathepsin-cleavage fluorescence probe. HepG2 cells were incubated with equimolar conjugate:LysoLight IgE (Thermo Fisher LysoLight Deep Red) at indicated concentrations; deep red fluorescence observed from cleavage of IgE in the lysosome was tracked using live cell imaging over three days and quantified as the area under the curve of fluorescence signal over time, normalized to signal from unconjugated parent protein (Oma S35H/Y57H-LC). Background degradation of unconjugated control was significantly higher at 100 nM than at 3.7 nM due to increased nonspecific internalization from pinocytosis, leading to smaller fold changes of conjugates versus control.

TABLE 28

| Oma | Normalized | |
|---|---|---|
| S35H/Y57H- | Fluorescence | |
| LC Conjugate | 100 nM | 3.7 nM |
| Unconjugated | 1.0 | 1.0 |
| 2318 | 2.1 | 4.1 |
| 2323 | 3.6 | 10.2 |
| 2328 | 2.5 | 4.4 |
| 2326 | 3.7 | 17.2 |
| 2306 | 1.8 | 5.6 |
| 2325 | 3.5 | 15.8 |
| 2313 | 4.1 | 13.0 |
| 2327 | 2.7 | 24.0 |
| 2317 | 4.2 | 15.2 |
| 2314 | 3.9 | 13.9 |
| 2315 | 4.0 | 15.6 |
| 2316 | 3.7 | 13.4 |
| 2321 | 4.1 | 13.2 |
| 2320 | 2.7 | 6.4 |
| 2329 | 3.0 | 11.6 |
| 2330 | 2.4 | 8.1 |
| 2310 | 2.4 | 4.3 |
| 2311 | 2.2 | 2.2 |
| 2401-C | 1.8 | 1.1 |
| 2402-C | 2.1 | 2.2 |
| 2322 | 3.5 | 7.8 |
| 2319-C | 1.0 | 1.1 |
| 2312 | 4.2 | 11.1 |
| 2308 | 2.3 | 6.7 |
| 2305 | 2.2 | 8.1 |
| 2304 | 4.0 | 12.7 |
| 2303 | 3.7 | 10.2 |
| 2309 | 3.1 | 6.7 |
| 2307 | 4.1 | 7.9 |
| 2324 | 2.6 | 5.8 |

### Example 8: Cycling of ASGPR-Based Conjugates

HepG2 cells (about 200,000) were plated in 12-well plates 48 hours before performing the experiment. The day of the experiment, cells were incubated with the conjugate at indicated concentrations for 1 hour. In most forms of the assay, the conjugates were labelled with Alexa Fluor647 dyes to control for total uptake of conjugate after the hour incubation period. Cells were then placed on ice to prevent trafficking/recycling during wash steps, and washed once with DPBS, once with sodium acetate buffer (20 mM, 150 mM NaCl, pH 5.2), once with Earle's Balanced Salt Solution (EBSS) adjusted to pH 2.5, and once again with DPBS, to remove the conjugate from media and cell surface. Cells were then detached from plate with 200 ul of 0.25% Trypsin/EDTA. After 10 minutes on ice, 800 ul of prewarmed (to 37° C.) EMEM with 1 μg/ml of human a-IgG antibody conjugated to Dylight 488 fluorophores was added to cells, which were placed in Eppendorf tubes and placed at 37 degrees. At indicated timepoints (0, 15, 30, 45, 60, and 90 minutes) 150 ul of cells in EMEM were removed from tube and placed in prechilled Eppendorf tube on ice. After collected all time points, cells were pelleted in 96-well conical bottom plates, washed once with FACS Buffer with BSA, then resuspended in FACS Buffer with BSA. The amount of conjugate capable of cycling to the cell surface and binding to the Dylight 488 labelled a-IgG was measured by analyzing median cellular 488 fluorescence intensity by flow cytometry.

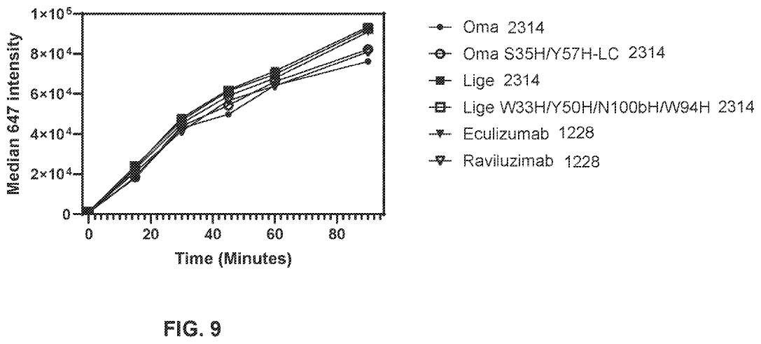

FIG. 9 demonstrates cycling of various ASGPR-based conjugates. HepG2 cells were treated with 50 nM conjugate (comprising wild-type antibody or pH-sensitive mutant, Oma S35H/Y57H-LC and Lige W33H/Y50H/N100bH/W94H), washed to remove excess conjugate, and incubated with Alexa Fluor 647-labelled a-human IgG to quantify amount of conjugate that recycles to the cell surface. Cell aliquots were collected every 15-30 minutes for quantification by FACS.

### Example 9: Cycling of M6PR-Based Conjugates

In accordance with Example 8, HepG2 cells were treated with 100 nM Oma conjugated to M6PR-based conjugates (i.e. Oma-XB105 and Oma-2403), washed to remove excess conjugate, and incubated with Alexa Fluor 647-labelled a-human IgG to quantify amount of conjugate that cycles to the cell surface (is externalized from the cell). Cell aliquots were collected every 15 minutes for quantification by FACS.

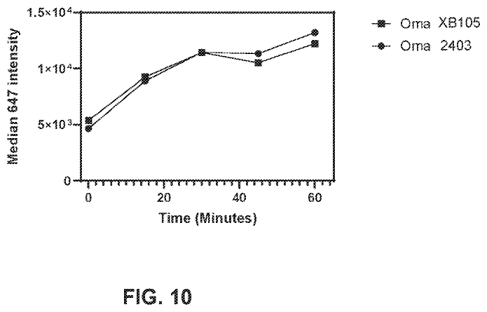

FIG. 10 demonstrates cycling of various M6PR-based conjugates.

### Example 10: Cycling Via FcRn Binding

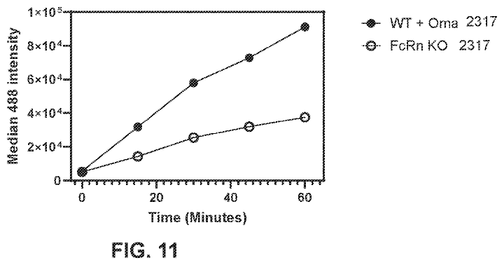

Referring to FIG. 11, HepG2 wild type or FcRn KO (FcRn knockout) cells were treated with 50 nM conjugate (i.e. Oma-2317), washed to remove excess conjugate, and incubated with Alexa Fluor 647-labelled a-human IgG to quantify amount of conjugate that recycles to the cell surface. Cell aliquots were collected every 15-30 minutes for quantification by FACS.

### Example 11: Externalization of Conjugates from a Cell Following Internalization (Cycling)

The method was also adapted to a high-throughput format. In this assay, 25,000 HepG2 cells were plated in 96-well plates 48 hours before performing the experiment. The day of the experiment, cells were incubated with conjugate at indicated concentrations for 1 hour. Cells were then placed on ice to prevent trafficking/recycling during wash steps, and washed once with DPBS, once with sodium acetate buffer (20 mM, 150 mM NaCl, pH 5.2), once with Earle's Balanced Salt Solution (EBSS) adjusted to pH 2.5, and once again with DPBS, to remove conjugate from media and cell surface. Afterwards, 100 ul of prewarmed (to 37° C.) EMEM with 1 μg/ml of human a-IgG antibody conjugated to Alexa Fluor647 fluorophores was added to cells for 1 hour before washing with DPBS and detaching with 0.25% Trypsin/EDTA. Cells were pelleted in 96-well conical bottom plates, washed once with FACS Buffer with BSA (Rockland Immunochemicals), resuspended FACS Buffer with BSA and median AF647 intensity was analyzed by flow cytometry. The amount of conjugate capable of cycling to the cell surface (being externalized from the cell) and binding to the Alexa Fluor647 labelled α-IgG was measured by analyzing median cellular 647 fluorescence intensity by flow cytometry. HepG2 cells were treated with 50 nM conjugate or unconjugated parent protein (Oma S35H/Y57H-LC), treated with stringent washed to remove conjugate from cell surface and media, and then incubated with fluorescent Alexa Fluor 647-labelled a-human IgG to quantify amount of conjugate that recycles back to the cell surface using FACS. Cellular fluorescence signal was normalized to unconjugated parent protein. Reported values are the average of the values of two replicate wells. Data shown in Table 29 demonstrate the ability of conjugates to cycle to the cell surface, i.e. to be externalized from a cell after being internalized.

TABLE 29

| Oma S35H/ | |
|---|---|
| Y57H-LC | Normalized |
| Conjugate | Fluorescence |
| Unconjugated | 1.0 |
| 2318 | 27.4 |
| 2323 | 43.2 |
| 2328 | 15.1 |
| 2326 | 57.7 |
| 2306 | 23.1 |
| 2325 | 54.2 |
| 2313 | 36.4 |
| 2327 | 71.2 |
| 2317 | 48.8 |
| 2314 | 40.5 |
| 2315 | 48.8 |
| 2316 | 45.7 |
| 2321 | 49.3 |
| 2320 | 24.1 |
| 2329 | 40.3 |
| 2330 | 42.9 |
| 2310 | 16.6 |
| 2311 | 11.6 |
| 2401-C | 3.2 |
| 2402-C | 10.5 |
| 2322 | 46.8 |
| 2319-C | 5.0 |
| 2312 | 44.5 |
| 2308 | 31.6 |
| 2305 | 44.7 |
| 2304 | 82.2 |
| 2303 | 52.7 |
| 2309 | 27.1 |
| 2307 | 42.9 |
| 2324 | 32.2 |

### Example 12: Degradation of Initial and Subsequent Dose of Target

HepG2 cells (25,000) were plated in 96-well plates 48 hours before performing the experiment. On the day of the experiment, indicated conjugate was diluted in fresh EMEM as a 2× stock, and mixed with an equimolar stock of human IgE (Enzo Life Sciences, BPD-DIA-HE 1) conjugated to a cathepsin-cleavable quenched fluorescent probe. The precomplexed conjugate:IgE mixture was incubated at 37 degrees Celsius for 30 minutes. Media from HepG2 was removed, and conjugate:IgE mixture in EMEM was added to cells for 2 hours. Cells were then placed on ice to prevent trafficking/recycling during wash steps, and washed once with DPBS, once with sodium acetate buffer (20 mM, 150 mM NaCl, pH 5.2), once with Earle's Balanced Salt Solution (EBSS) adjusted to pH 2.5, and once again with DPBS, to remove conjugate from media and cell surface. Afterwards, 100 ul of prewarmed (to 37 degrees Celsius) EMEM with cathepsin-cleavable quenched fluorescent probe-conjugated IgE was added to cells for 4 hours before washing with DPBS and detaching with 0.25% Trypsin/EDTA. Cells were pelleted in 96-well conical bottom plates, washed once with FACS Buffer with BSA (Rockland Immunochemicals), resuspended FACS Buffer with BSA and median 488 and 647 intensity was analyzed by flow cytometry. Delivery of labelled IgE to the lysosome and subsequent cleavage of initial precomplexed IgE was measured by analyzing median cellular 647 intensity, while cleavage of the post wash addition of IgE, which should only be degraded by recycling conjugates which released the first round of precomplexed IgE, was analyzed by median cellular 488 intensity. HepG2 cells were treated with human IgE (Enzo Life Sciences, BPD-DIA-HE1) conjugated to a cathepsin-cleavable quenched deep red (647) fluorescent probe precomplexed to conjugate fortwo hours, then washed, and fresh media with IgE conjugated to a green (488) cathepsin-cleavable fluorescent probe was then added without conjugate. Deep red fluorescence demonstrates the ability of conjugate to internalize into a cell and facilitate degradation of a target in a lysosome. Green fluorescence demonstrates the ability of the conjugate to externalize from the cell, i.e. cycle to the cell surface and degrade a subsequently dosed target. Fluorescence values were measured by FACS and normalized to unconjugated parent protein. Reported values are the average of the values of two replicate wells.

TABLE 30

| Oma | Normalized Fluorescence | |
|---|---|---|
| S35H/ | Precomplex | Post Wash |
| Y57H-LC | Median | Median |
| Conjugate | Deep Red | Green |
| Unconjugated | 1.0 | 1.0 |
| 2318 | 2.4 | 2.0 |
| 2323 | 3.2 | 2.8 |
| 2328 | 2.4 | 1.8 |
| 2326 | 4.1 | 4.1 |
| 2306 | 2.5 | 2.4 |
| 2325 | 3.9 | 3.6 |
| 2313 | 3.5 | 3.2 |
| 2327 | 3.7 | 4.6 |
| 2317 | 3.6 | 3.7 |
| 2314 | 3.6 | 3.5 |
| 2315 | 3.3 | 3.1 |
| 2316 | 3.8 | 3.5 |
| 2321 | 4.0 | 3.7 |
| 2320 | 3.2 | 2.7 |
| 2329 | 3.3 | 3.0 |
| 2330 | 3.0 | 2.9 |
| 2310 | 2.3 | 2.0 |
| 2311 | 2.0 | 1.7 |
| 2401-C | 1.7 | 1.2 |
| 2402-C | 2.1 | 1.6 |
| 2322 | 3.5 | 3.3 |
| 2319-C | 1.2 | 1.1 |
| 2312 | 3.5 | 3.0 |
| 2308 | 3.3 | 2.8 |
| 2305 | 3.3 | 2.9 |
| 2304 | 3.9 | 4.1 |
| 2303 | 3.4 | 3.6 |
| 2309 | 3.0 | 2.5 |
| 2307 | 3.7 | 3.5 |
| 2324 | 3.2 | 2.9 |
| Oma WT 2314 | 5.7 | 2.3 |

### Example 13: Degradation of Super-Stoichiometric Levels of Target

For evaluation of compounds in a high throughput format, 25,000 HepG2 cells were plated in a 96-well plate. The following day, 50 ul of 1 nM conjugate and 25 nM recombinant IgE in EMEM were added to the cells. 50 ul of the initial stock of conjugate:IgE was saved to quantify for an initial time point. Three days later, the media was collected from cells, and both initial and 72 hour time points were quantified for IgE concentration using the MSD assay.

HepG2 cells were incubated in media containing 1 nM conjugate and 25 nM IgE for three days. Concentrations of IgE at beginning and end of experiment were quantified by ELISA-based assay. Molar equivalents of IgE depleted relative to conjugate added are shown, calculated as (A-B)/C, where A is the initial concentration of IgE, B is the concentration of IgE at day three, and C is the concentration of conjugate. Reported values are the average of the values of two replicate wells. Negative values reflect evaporation of media during time course of the experiment, artificially increasing the concentration of IgE at the end of the experiment relative to the starting point; volume of media and rate of evaporation of media was consistent across all wells during the time course of the experiment.

TABLE 31

| Oma | Average | |
|---|---|---|
| S35H/ | Degradation | |
| Y57H-LC | (Molar | Standard |
| Conjugate | equivalents) | deviation |
| Unconjugated | −1.4 | 1.6 |
| 2318 | 4.2 | 2.4 |
| 2323 | 4.4 | 1.8 |
| 2328 | −0.3 | 0.2 |
| 2326 | 9.0 | 4.7 |
| 2306 | 1.8 | 3.4 |
| 2325 | 6.9 | 2.2 |
| 2313 | 7.5 | 4.5 |
| 2327 | 9.2 | 1.3 |
| 2317 | 4.0 | 3.6 |
| 2314 | 6.5 | 2.9 |
| 2315 | 4.5 | 0.2 |
| 2316 | 5.7 | 0.3 |
| 2321 | 2.4 | 0.9 |
| 2320 | 0.5 | 2.3 |
| 2329 | 4.7 | 1.9 |
| 2330 | 2.8 | 1.3 |
| 2310 | 4.0 | 1.8 |
| 2311 | 0.5 | 1.8 |
| 2401-C | −1.0 | 0.1 |
| 2402-C | 0.7 | 1.2 |
| 2322 | 5.2 | 0.8 |
| 2319-C | 2.1 | 4.2 |
| 2312 | 4.1 | 0.6 |
| 2308 | 2.5 | 2.7 |
| 2305 | 7.0 | 5.1 |
| 2304 | 8.8 | 0.4 |
| 2303 | 3.6 | 1.7 |
| 2309 | 4.1 | 1.1 |
| 2307 | 5.6 | 2.9 |
| 2324 | 5.2 | 2.8 |
| Oma WT 2314 | −1.0 | 2.2 |

### Example 14: Mouse Model with Constant Production of Human IgE (hIgE)

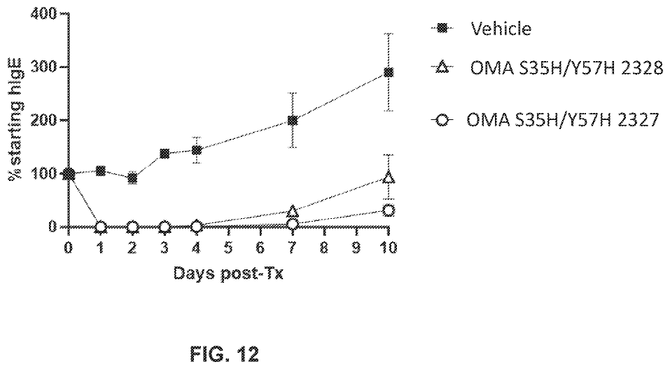

Referring to FIG. 12, 3×10⁶ U266B 1 myeloma cells were implanted subcutaneously into the flank of NOD-scid IL2Rgamma-null mice. Tumor growth and hIgE titers, via R-PLEX Meso Scale Discovery Human IgE Assay, were monitored for at least 14 days to ensure successful myeloma implantation. Prior to administration of test articles, mice were divided into experimental cohorts with normalized levels of hIgE. Conjugates (Oma S35H/Y57H 2328 and Oma S35H/Y57H 2327) and controls (PBS buffer vehicle only) were then dosed subcutaneously into the scruff of the neck at 10 mg/kg. Blood was collected at indicated timepoints, with serum subsequently tested for depletion of hIgE.

### Example 15: Degradation of Complement C5

In view of the above-noted synthetic examples, following conjugates were prepared: eculizumab and 2405 (Ecu-2405); eculizumab and 1117 (Ecu-1117); ALXN1210 and 2405 (ALXN1210-2405); and ALXN1210 and 1117 (ALXN1210-1117). ALXN1210 and eculizumab contained YTE and L443C mutations. 2405

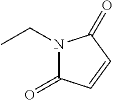

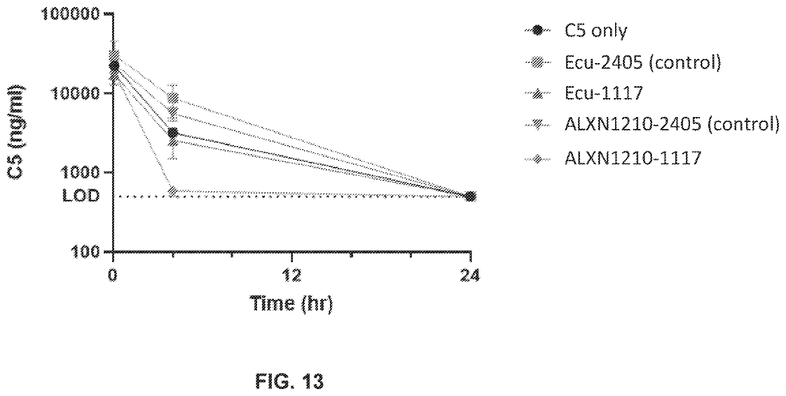

is commercially available (CAS No. CAS: 128-53-0). Referring to FIG. 13, C57BL/6 mice were dosed intravenously with 0.5 mg/kg of indicated conjugate (n=3/group). 24 hours later, 1 mg/kg of hC5 was administered intravenously. Blood was collected and serum isolated at 5m, 4 hr, and 24 hr following administration of hC5. Levels of hC5 were assessed at each timepoint via ELISA.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A conjugate that comprises a ligand moiety, X, conjugated via a linker, L, to a target-binding moiety, Y,
wherein
the ligand moiety, X, binds a lysosomal targeting molecule extracellularly;
the target-binding moiety, Y, binds a target molecule extracellularly,
the target-binding moiety, Y, dissociates from the target molecule intraendosomally; and
the conjugate is externalized from a cell.

2. The conjugate of claim 1, wherein ligand moiety, X, remains bound to the lysosomal targeting molecule intraendosomally.

3. The conjugate of claim 1 or 2, wherein the target-binding moiety, Y, binds FcRn intraendosomally.

4. The conjugate of any one of claims 1-3, wherein the conjugate dissociates from the lysosomal targeting molecule intraendosomally.

5. The conjugate of claim 1, wherein the lysosomal targeting molecule is ASGPR.

6. The conjugate of claim 1, wherein Y is an antibody or antibody fragment.

7. The conjugate of claim 2, wherein the conjugate is externalized from the cell via the lysosomal targeting molecule.

8. The conjugate of claim 2, wherein the ligand moiety, X, has an equal binding affinity for the lysosomal targeting molecule extracellularly and intraendosomally.

9. The conjugate of claim 2, wherein the ligand moiety, X, has an equal binding affinity for the lysosomal targeting molecule at an extracellular pH and at an intraendosomal pH.

10. The conjugate of claim 2, wherein the ligand moiety, X, has an equal binding affinity for the lysosomal targeting molecule at an extracellular Ca²⁺ concentration and at an intraendosomal Ca²⁺ concentration.

11. The conjugate of claim 3, wherein the conjugate is externalized from the cell via FcRn.

12. The conjugate of claim 4, wherein the target-binding moiety, Y, binds FcRn intraendosomally.

13. The conjugate of claim 12, wherein the conjugate is externalized from the cell via FcRn.

14. The conjugate of claim 12, wherein the ligand moiety, X, has a higher binding affinity for the lysosomal targeting molecule extracellularly than intraendosomally.

15. The conjugate of claim 12, wherein the ligand moiety, X, has a higher binding affinity for the lysosomal targeting molecule at an extracellular pH than at an intraendosomal pH.

16. The conjugate of claim 12, wherein the ligand moiety, X, has a higher binding affinity for the lysosomal targeting molecule at an extracellular Ca²⁺ concentration than at an intraendosomal Ca²⁺ concentration.

17. The conjugate of claim 12, wherein the target-binding moiety, Y, has a higher binding affinity for FcRn intraendosomally than extracellularly.

18. The conjugate of claim 12, wherein the target-binding moiety, Y, has a higher binding affinity for FcRn at an intraendosomal pH than at an extracellular pH.

19. The conjugate of claim 12, wherein the target-binding moiety, Y, has enhanced binding to FcRn relative to wild-type at an endosomal pH.

20. The conjugate of claim 12, wherein the target-binding moiety, Y, has approximately equal binding affinity to FcRn extracellularly as wild-type IgG does extracellularly.

21. The conjugate of claim 12, wherein the target-binding moiety, Y, has, at pH 7.4, approximately equal binding affinity to FcRn as wild-type IgG.

22. The conjugate of claim 12, wherein Y is an antibody.

23. The conjugate of claim 1, wherein the target-binding moiety, Y, has a higher binding affinity for the target molecule extracellularly than intraendosomally.

24. The conjugate of claim 1, wherein the target-binding moiety, Y, has a higher binding affinity for the target molecule at an extracellular pH than at an intraendosomal pH.

25. The conjugate of claim 1, wherein the target-binding moiety, Y, has a pH 6.0:7.4 K_{D} ratio for the target molecule of between 2:1 and 10,000:1.

26. The conjugate of claim 1, wherein the target-binding moiety, Y, has a higher k_{off} rate for the target molecule intraendosomally than extracellularly.

27. The conjugate of claim 1, wherein the conjugate is capable of cycling for a period of hours to days.

28. The conjugate of claim 1, wherein the target-binding moiety, Y is an antibody or antibody fragment that has been mutated from the wild-type with one or more histidine substitutions.

29. The conjugate of any preceding claim, wherein the conjugate is of formula (I′): 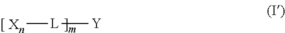
or a prodrug thereof, or a or a pharmaceutically acceptable salt thereof,
wherein:
n is 1 to 500;
m is 1 to 20;
X is the ligand moiety; and
Y is the target-binding moiety.

30. The conjugate of any preceding claim, wherein the lysosomal targeting molecule is a cell surface receptor that provides for internalization of the conjugate.

31. The conjugate of any preceding claim, wherein the lysosomal targeting molecule is selected from asialoglycoprotein receptor (ASGPR), cation independent mannose-6-phosphate receptor (CI-M6PR also referred to herein as M6PR), folate receptor, LDLR, CD63, sortilin, IFITM3, molecules in the endosome/lysosome pathway, LIMP-1, and LIMP-2.

32. The conjugate of any preceding claim, wherein X is a moiety that binds ASGPR or M6PR.

33. The conjugate of any preceding claim, wherein X is a moiety that binds ASGPR.

34. The conjugate of claim 33, wherein the conjugate is of formula (I′): 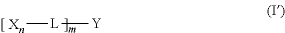
or a prodrug thereof, or a pharmaceutically acceptable salt thereof,
wherein:
n is 1 to 500;
m is 1 to 20;
L is a linker; and
X is an asialoglycoprotein receptor (ASGPR) binding moiety of formula (II): 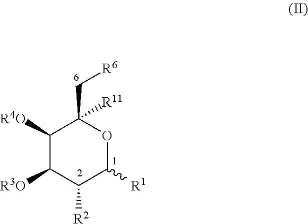
wherein:
R¹ is selected from —Z¹—*, —H, —OH, optionally substituted (C₁-C₆)alkyl, —OCH₃, —OCH₂CH═CH, optionally substituted —S—(C₁-C₆)alkyl, optionally substituted aryl, optionally substituted heteroaryl, optionally substituted —S-aryl, and optionally substituted —S-heteroaryl;
R² is selected from —Z¹—*, —NHCOCH₃, —NHCOCF₃, —NHCOCH₂CF₃, —OH, —NHR, and optionally substituted triazole;
R⁶ is selected from —Z¹—*, —OH, —OR, optionally substituted (C₁-C₆)alkyl, —OC(O)R, —C(O)NHR, —NR^{xx}R^{yy}, optionally substituted aryl, optionally substituted heteroaryl, —NHCOR, and —NRCOR;
each R is independently optionally substituted (C₁-C₆)alkyl, optionally substituted aryl, or optionally substituted heteroaryl;
R^{xx} and R^{yy} are independently H, optionally substituted (C₁-C₆)alkyl, or R^{xx} and R^{yy} can cyclize to form an optionally substituted heterocyclyl;
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, - CONR²¹—, —NR²¹SO₂—, —SO₂NR²¹—, —NR²¹C(═O)NR²¹—, and —NR²¹C(═S)NR²¹—;
—Z¹¹— is —O—, —S—, —N(R²¹)—, or —C(R²²)₂; provided that when R¹ is-Z¹—*, and Z¹ is —Z¹¹—, then —Z¹¹— is not —O—;
-A¹- and -A²- are optionally substituted heterocyclylene, optionally substituted arylene, or optionally substituted heteroarylene;
each R²¹ is independently selected from H, optionally substituted (C₁-C₆)alkyl, —COR, and optionally substituted heteroaryl; and
each R²² is independently selected from H, halogen, and optionally substituted (C₁-C₆)alkyl.

35. The conjugate of claim 34, wherein -L-Y comprises: 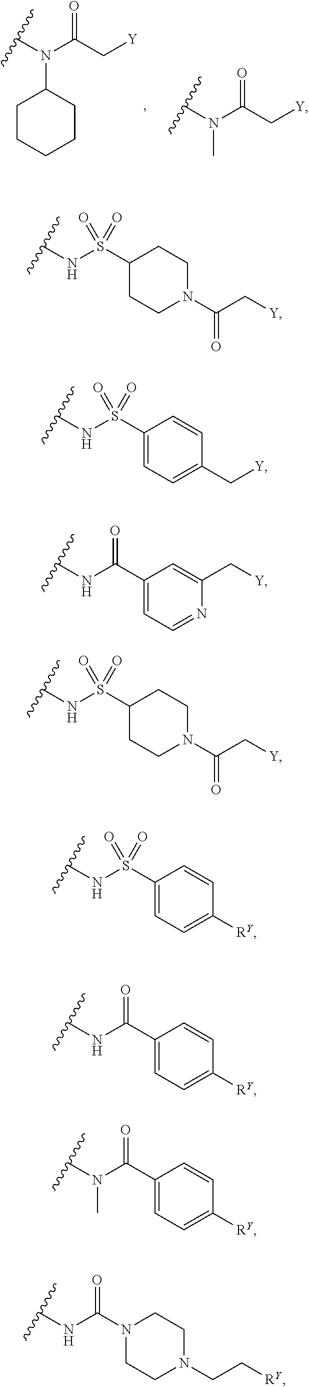 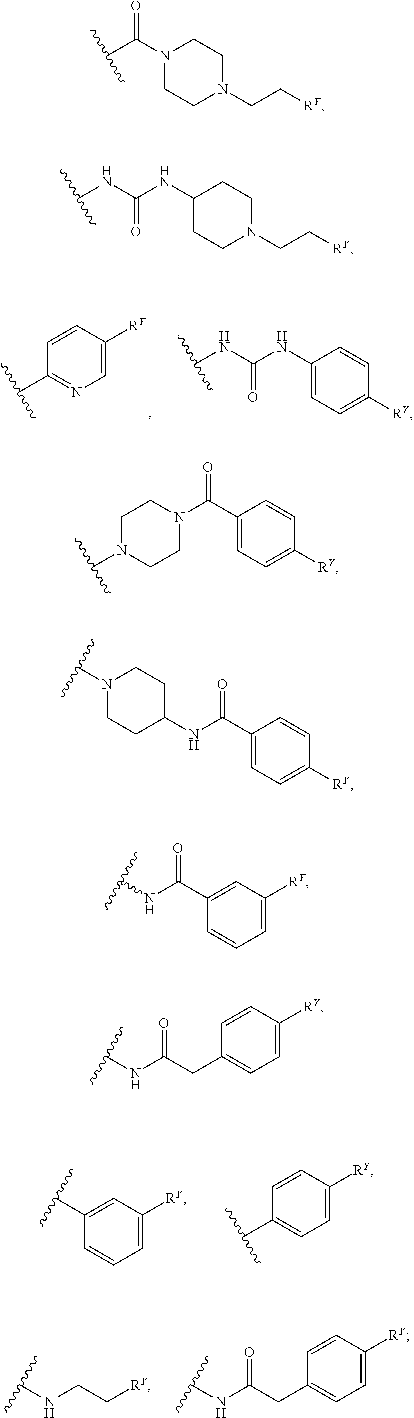
wherein R^{Y} is

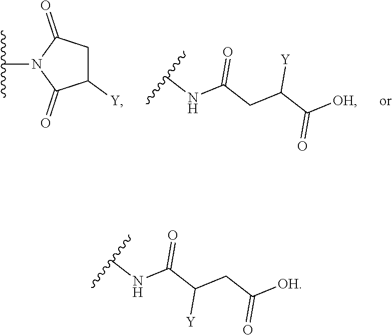

36. The conjugate of claim 34 or 35, wherein X is represented by formula (a-II):

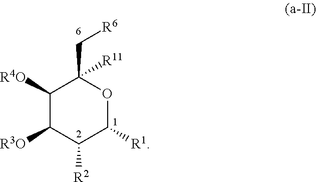

37. The conjugate of any one of claims 34-36, wherein R¹ is —Z¹—*, —H, or (C₁-C₆)alkyl.

38. The conjugate of any one of claims 34-36, wherein R² is —Z¹—* or —NHCOCH₃.

39. The conjugate of any one of claims 34-38, wherein R³ and R⁴ are each —H.

40. The conjugate of claim 32, wherein X is a moiety that binds to M6PR.

41. The conjugate of claim 40, wherein X is of formula (IV): 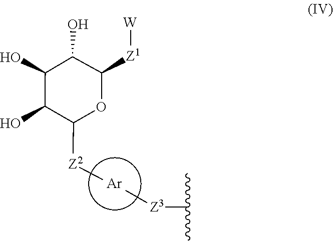
wherein:
W is a non-hydrolyzable hydrophilic head group;
Z¹ is selected from optionally substituted (C₁-C₃)alkylene and optionally substituted ethenylene;
Z² is selected from S, NR²¹ and C(R²²)₂, wherein each R²¹ is independently selected from H, and optionally substituted (C₁-C₆)alkyl, and each R²² is independently selected from H, halogen and optionally substituted (C₁-C₆)alkyl;
each A is independently an optionally substituted aryl or heteroaryl linking moiety; and
each Z³ is independently a linking moiety.

42. The conjugate of claim 41, wherein Z² is S.

43. The conjugate of claim 41 or 42, wherein W is phosphonate, thiophosphonate, carboxylic or malonic acid, or a salt thereof.

44. The conjugate of any one of claims 45-47, wherein X is: 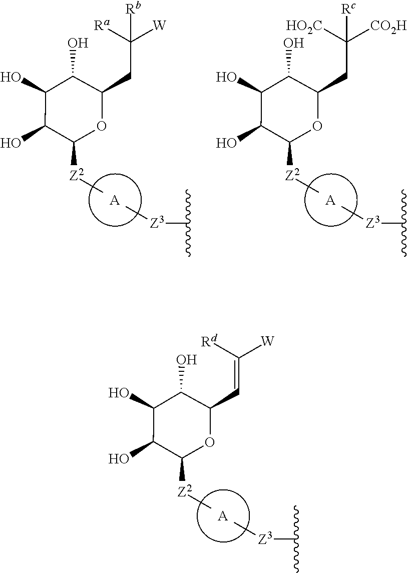
wherein R^{a}, R^{b}, R^{c} and R^{d} are independently H or F.

45. The conjugate of any one of claims 41-44, wherein X is: 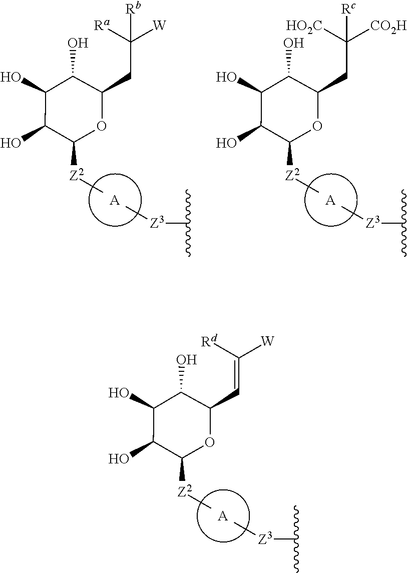
wherein R^{a}, R^{b}, R^{c} and R^{d} are independently H or F.

46. The conjugate of any one of claims 41-45, wherein A is optionally substituted aryl or optionally substituted heteroaryl, preferably A is independently selected from optionally substituted phenyl, optionally substituted pyridyl, optionally substituted biphenyl, optionally substituted naphthalene, optionally substituted triazole and optionally substituted phenylene-triazole.

47. The conjugate of any preceding claim, wherein L comprises of 10 to 60 consecutive branched or linear chain atoms.

48. The conjugate of any preceding claim, wherein L is of formula (IIb′): 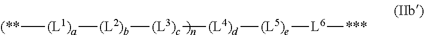
wherein:
n is 1, 2, or 3;
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

49. The conjugate of claim 48, wherein each L¹ to L⁵ independently comprises one or more linking moieties independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50;
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹⁶—, —C₁₋₆-alkylene-NR¹⁶C(S)NR¹⁶—, —O(CH₂)ₚ—, —(OCH₂CH₂)ₚ—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹—; and
each R¹⁶ is independently —H, optionally substituted (C₁-C₆)alkyl, optionally substituted aryl, optionally substituted monocyclic heteroaryl or monocyclic heteroaryl.

50. The conjugate of claim 48, wherein each L¹ to L⁵ is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50; and 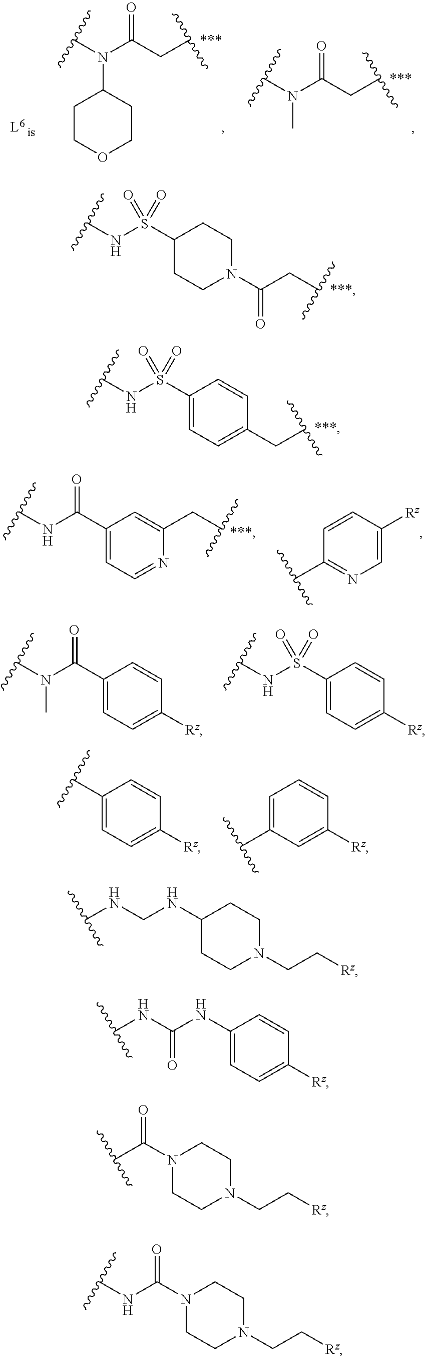 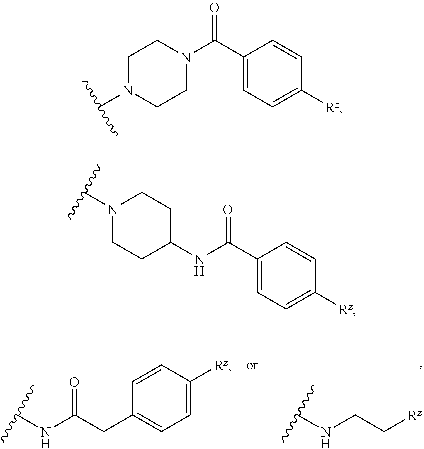
wherein R^{z} is

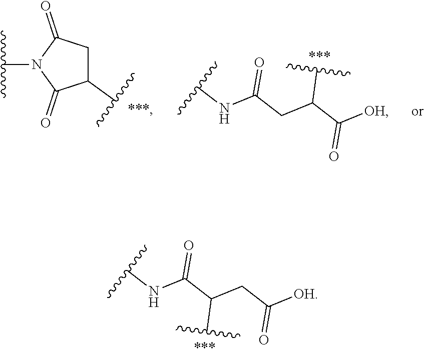 [SMILES: *C1CC(=O)N(*)C1=O ; *NC(=O)CC(*)C(=O)O ; *NC(=O)C(*)CC(=O)O]

50. A method of degrading a target molecule in a subject in need thereof, comprising administering an effective amount of a conjugate of any preceding claim to the subject.

51. The method of claim 50, wherein at least 90% of the target is degraded at four days following the administration.

52. The method of claim 50, wherein at least 90% of the target is degraded at seven days following the administration.

53. The method of claim 50, wherein the extracellular concentration of the target is substantially maintained for a time period of at least four days, at an amount of at least 90% less than the initial extracellular concentration of the target prior to administering an effective amount of the conjugate.

54. The method of claim 50, wherein the time period is seven days or more.

55. The method of any one of claims 50-54, wherein a super-stoichiometric target:conjugate ratio is degraded.

56. The method of claim 55, wherein the ratio is at least about 5.

57. The method of any one of claims 50-56, wherein the target-binding moiety, Y, has a higher binding affinity for the target molecule extracellularly than intraendosomally.

58. The method of any one of claims 50-57, wherein the target is IgE.

59. A method of degrading a target molecule in a subject in need thereof, comprising administering an effective amount of a conjugate that comprises:
a means for binding a lysosomal targeting molecule extracellularly;
a means for binding a target molecule extracellularly;
a means for dissociating from the target molecule intraendosomally; and
wherein the conjugate is externalized from a cell.

60. The method of claim 59, wherein the means for binding a lysosomal targeting molecule, remains bound to the lysosomal targeting molecule intraendosomally.

61. The method of claim 59 or 60, wherein the means for binding a target molecule also binds FcRn intraendosomally.

62. The method of claim 61, wherein the conjugate dissociates from the lysosomal targeting molecule intraendosomally.
